(19) United States
(12) Patent Application Publication
(10) Pub. No.: US 2024/0366754 A1
(43) Pub. Date: Nov. 7, 2024

(54) PHARMACEUTICAL COMPOSITIONS FOR DELIVERY OF VIRAL ANTIGENS AND RELATED METHODS

(71) Applicant: BioNTech SE, Mainz (DE)

(72) Inventors: Richard B. Gaynor, Carmel, IN (US); Michael Steven Rooney, Lexington, MA (US); Asaf Poran, Stoneham, MA (US); Theresa Addona, Needham, MA (US); Scott Goulding, Malden, MA (US); Ekaterina Esaulova, Boston, MA (US); Miles Kirsch, Beacon, NY (US); Yunpeng Liu, Cambridge, MA (US); Lauren Stopfer, Cambridge, MA (US)

(21) Appl. No.: 18/701,119

(22) PCT Filed: Oct. 14, 2022
(86) PCT No.: PCT/US22/46799

§ 371 Date: Apr. 12, 2024

Related U.S. Application Data
(60) Provisional application No. 63/360,623, filed on Oct. 15, 2021.

(51) Int. Cl. A61K 39/25; A61K 39/00; A61K 39/12; A61K 39/245; A61P 37/04; C07K 14/005

(52) U.S. Cl. CPC A61K 39/25; A61K 39/12; A61K 39/245; A61P 37/04; C07K 14/005; A61K 2039/53; A61K 2039/54; A61K 2039/575; A61K 2039/70; C12N 2710/16122; C12N 2710/16134; C12N 2710/16722; C12N 2710/16734; C12N 2770/16022; C12N 2770/16034

(57) ABSTRACT

The present disclosure provides pharmaceutical compositions for delivery of viral antigens (e.g., a viral vaccine) and related technologies (e.g., components thereof and/or methods relating thereto).

### CROSS REFERENCE

The present application claims priority to United States Provisional Patent Application Nos. 63/360,623 filed Oct. 15, 2021, the entire contents of which, including the associated sequence listing, are hereby incorporated by reference in their entirety.

### SEQUENCE LISTING

The instant application contains a Sequence Listing which has been submitted via DVD-R. Said DVD-R, recorded on Oct. 14, 2022, is labeled 2013237-0466-SL.zip and contains a 664,937,000 byte file.

### BACKGROUND

Viral infections represent a major threat to human health and well-being. In particular, certain viruses can result in chronic infection, e.g., via a latent phase. Viruses, such as HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus, can all result in short term and long term complications.

For example, human alphaherpesvirus 3 (commonly referred to as varicella-zoster virus (VZV)) causes both varicella (chickenpox) by primary infection and herpes zoster (HZ or shingles) by endogenous reactivation from latency. Congenital varicella syndrome is a rare condition that can result from VZV infection in pregnant women infected during the first 20 weeks of gestation. According to the World Health Organization, VZV is highly contagious, and in the absence of an effective vaccination program, affects nearly every person worldwide by mid-adulthood.

Human betaherpesvirus 5 is commonly referred to as human cytomegalovirus (CMV) and is ubiquitous among humans worldwide. For example, previous studies have estimated a global CMV seroprevalence of 83% in the general population, 86% in women of childbearing age, and 86% in donors of blood or organs. According to the Centers for Disease Control and Prevention, in the United States, nearly one in three children are infected with CMV by age five. Over half of adults have been infected with CMV by age 40. Once CMV is in a person's body, it stays there for life and can reactivate. Typically, CMV infection does not produce symptoms when it causes primary infection, reinfection, or reactivation because these three types of infection are all controlled by normal immune system responses. However, CMV becomes an important pathogen in individuals whose immune system is immature or compromised, such as the immune systems of unborn children in which CMV can result in congenital CMV disease.

Noroviruses are the leading cause of epidemic gasteroentetitis in humans of all age groups, and are often responsible for outbreaks, for example in schools, hospitals, residential facilities, cruise ships, the military, etc. According to the World Health Organization (“WHO”), about one in every five cases of acute gastroenteritis (inflammation of the stomach or intestines) that leads to diarrhea and vomiting is caused by a norovirus. The WHO has estimated that healthcare costs and lost productivity due to norovirus infections worldwide cost $60 billion annually.

Thus, there remains a need for compositions and methods that prevent and/or treat viral infections.

### SUMMARY

Many viral infections clear from a host's body in a few days to weeks. However, certain viruses can cause an initial lytic infection (active infection) during which time the virus actively replicates, followed by a period of latency during which the virus is inactive or dormant and does not actively replicate or multiply. Such viruses that enter a latent phase after the initial infection are sometimes called latent viruses and do not kill the host cells during the latent viral phase. However, the viral genome (genetic material) and infectious virus particles of the latent viruses remain in the body. Such latent viral infections can be reactivated at a later time, sometimes even years after the initial infection, into a lytic form (active virus replication).

The reactivation of latent viruses to re-enter the lytic phase can occur when the immune system of an infected host is weakened, for example, by external and/or internal factors including, e.g., infections by other viruses, trauma, fever, or treatment with immunosuppressive or anticancer therapy. Examples of latent viruses that are known to cause typical latent infections in human include, but are not limited to viruses of the Herpesviridae, Papillomaviridae, Parvoviridae, or Adenoviridae families. Exemplary latent viruses include, but are not limited to HSV-1, HSV-2, VZV, Human Inmmunodeficiency Virus (HIV), Epstein-Barr Virus (EBV), CMV, HHV-6, HHV-7, KSHV (HHV-8), JC virus (JCV), BK virus (BKV), parvovirus, and adenovirus.

Many of the existing vaccines against viral infection primarily focus on eliciting neutralizing antibody response and/or targeting antigens expressed during a lytic infection in an effort to limit the initial infection with the virus. The present disclosure recognizes that antigens expressed during a lytic infection can be downregulated during the latency period, and thus vaccines that primarily target antigens expressed during a lytic infection can have a reduced impact on the latent infection or viral emergence from the latent phase. The present disclosure provides an insight that T cell response(s) to antigens expressed by a latent virus during the latency period or as it emerges from its latent phase may be particularly important and/or beneficial for treating subjects with a latent viral infection (e.g., by protecting such subjects from reactivation of a latent viral infection), and/or protecting subjects who have an initial infection from developing a latent viral infection. In one aspect, the present disclosure provides technologies (e.g., compositions and methods) to treat and/or prevent latent virus infection in subjects in need thereof by administering a composition that delivers one or more antigens (e.g., T-cell antigens) that are associated with a latent virus infection. For example, in some embodiments, a composition comprising at least a polyribnucleotide encoding a polypeptide that comprises one or more antigens (e.g., T-cell antigens) that are associated with a latent virus infection.

The present disclosure, among other things, also provides a recognition that reducing virus load during an active infection may be helpful to reduce the amount of virus that can potentially seed a latent virus infection. In some embodiments, the present disclosure provides a recognition that vaccination with antigens from both the lytic and latent stages of a viral infection may be helpful to limit the amount of virus that can potentially seed a latent virus infection and help clear or suppress latent virus. In one aspect, the present disclosure provides combination technologies (e.g., compositions and methods) to treat and/or prevent a virus infection in subjects in need thereof by administering a combination that delivers (a) one or more antigens (e.g., B cell antigens and/or T cell antigens) that are associated with a latent virus infection, and (b) one or more antigens (e.g., B cell antigens and/or T cell antigens) expressed during an active infection (e.g., one or more antigens expressed during an active infection that are exposed to the host's serum during the life cycle of the virus). In some embodiments, such latency-associated and active infection-associated antigens are delivered by polyribonucleotides. For example, in some embodiments, a combination that is useful to treat and/or prevent a virus infection in need thereof comprises: (a) a first pharmaceutical composition comprising a first polyribonucleotide, wherein the first polyribonucleotide encodes a first polypeptide, and the first polypeptide comprises one or more T-cell antigens that are associated with a latent virus infection; and (b) a second pharmaceutical composition comprising a second polyribonucleotide, wherein the second polyribonucleotide encodes a second polypeptide, and the second polypeptide comprises one or more antigens that are not associated with a latent virus infection. In some embodiments, antigens that are not associated with a latent virus infection are or comprise one or more antigens that are associated with an initial infection by the same virus. In some embodiments, antigens that are associated with an initial infection by the same virus are or comprise one or more antigens that are expressed during productive replication.

The present disclosure provides pharmaceutical compositions (e.g., immunogenic compositions, e.g., vaccines) for delivering viral antigen constructs to a subject (e.g., a patient) and related technologies (e.g., methods). In particular, the present disclosure provides viral vaccine compositions and related technologies (e.g., methods). In some embodiments, the present disclosure provides certain viral antigen constructs particularly useful in effective vaccination. In some embodiments, a viral antigen as described herein is an antigen comprising one or more epitopes of a viral protein from a virus of the Herpesviridae, Papillomaviridae, Parvoviridae, or Adenoviridae families. In some embodiments, a viral antigen as described herein is an antigen comprising one or more epitopes of a viral protein from HSV-1, HSV-2, VZV, Human Immunodeficiency Virus (HIV), Epstein Barr Virus (EBV), CMV, HHV-6, HHV-7, KSHV (HHV-8), JC virus (JCV), BK virus (BKV), parvovirus, and adenovirus.

Among other things, the present disclosure provides an insight that many prior strategies for developing pharmaceutical compositions (e.g., immunogenic compositions, e.g., vaccines) for treatment of and/or protection from viral infection have focused primarily, or even almost exclusively, on development of neutralizing antibodies that target surface glycoproteins. The present disclosure identifies a problem with such strategies including, for example, that they may fail to appreciate value or even criticality of ensuring that an induced immune response includes significant T cell activity (in some embodiments, CD4 T cell, in some embodiments CD8 T cell, in some embodiments, both). In some particular embodiments, the present disclosure provides an insight that T cell response(s) may be particularly important and/or beneficial for viruses that have a latent phase or otherwise can remain reasonably dormant (e.g., non-lytic) in a host (e.g., human) system. For example, Herpesviruses (e.g., CMV, HSV-1, HSV-2, VSV, etc) are believed to infect substantially all humans, and to establish lifelong latency (see, for example, Cohen J. Clin. Invest 130:3361, 2020; see also Forte et al., Cell Infect. Microbiol. Doi.org/10.3389/fcimb.2020.00130, 31 Mar. 2020). Latent infection by HIV has been described as the “main obstacle to curing HIV/AIDS (see, for example, Chanut Science News 30 Apr. 2020). The JC virus has been reported to be latent in kidneys and lymphoid organs, and possibly brains, of healthy individuals (see, for example, Tan et al., J. Virol 18:9200, September 2010). Even noroviruses, which are generally thought to be cleared quickly, have been reported to be able to establish long-term infections (see, for example, Capizzi et al., BMC Infect Dis 11:131, 30 Aug. 2011).

The present disclosure provides an insight that consideration of expression of viral polypeptides comprising one or more epitopes (e.g., at particular periods of the viral life cycle and/or in particular tissues or compartments of an infected subject) can improve vaccine effectiveness.

In some embodiments, the present disclosure provides technologies for identifying, selecting, and/or characterizing viral (e.g., HSV-1, HSV-2, VZV, Human Immunodeficiency Virus (HIV), Epstein Barr Virus (EBV), CMV, HHV-6, HHV-7, KSHV (HHV-8), JC virus (JCV), BK virus (BKV), parvovirus, or adenovirus) polypeptide or epitope sequences, and combinations thereof, particularly useful for inclusion in a pharmaceutical compositions (e.g., immunogenic compositions, e.g., vaccines) as described herein.

In some embodiments, pharmaceutical compositions (e.g., immunogenic compositions, e.g., vaccines) that comprise or deliver CD4 and CD8 epitope(s) of one or more viral (e.g., HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus) proteins, e.g., in addition to one or more B cell epitopes. Among other things, the present disclosure provides viral (e.g., HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus) antigen constructs and compositions (e.g., pharmaceutical compositions, e.g., immunogenic compositions, e.g., vaccines) that comprise and/or deliver antigen constructs that induce both neutralizing antibodies and T cells (e.g., CD4 and/or CD8 T cells), for example, targeting a viral glycoprotein and, in some embodiments, one or more additional viral proteins. In some embodiments, the present disclosure provides such constructs and compositions that induce particularly strong neutralizing antibody responses and/or particularly diverse T cell responses (e.g., targeting multiple T cell epitopes).

In some embodiments, the present disclosure provides such constructs and compositions that induce robust B cell responses. In some embodiments, a B cell response includes the production of a diverse, specific repertoire of antibodies.

In some embodiments, the present disclosure provides such constructs and compositions that induce T cell and B cell responses to viral (e.g., HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus) antigens and/or epitopes.

The present disclosure provides the recognition, for example, that constructs and compositions comprising RNA molecules as described herein (e.g., encoding for one or more viral antigens and/or epitopes) may result in a higher degree of antigen presentation to various immune system components and/or pathways. In some embodiments, administration of such constructs or compositions may induce T cell and/or B cell responses. The present disclosure provides the insight that, e.g., in some embodiments in which T cell and B cell responses are induced in a subject, the subject may have a more sustained, long-term immune response. Such an immune response can be beneficial, e.g., for preventing viral reactivation with a single administration, which may increase vaccination rates and subject compliance as compared with presently available vaccines that require dosing every few years. In some embodiments, constructs and compositions comprising RNA molecules as described herein (e.g., encoding for one or more viral antigens and/or epitopes) can provide more diverse protection (e.g., protection against viral variants) because, without wishing to be bound to any particular theory, the constructs and compositions can induce multiple immune system responses.

The present disclosure also provides the recognition that, by administering constructs and compositions that encode viral antigens and/or epitopes (e.g., HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus antigens and/or epitopes), the constructs and compositions described herein avoid administering viral virions, which may infect the subject, go into latency, and reactivate.

Still further, the present disclosure provides an insight (and also identifies a source of a problem in certain prior viral vaccination strategies) that, in some embodiments, particularly effective pharmaceutical compositions (e.g., immunogenic compositions, e.g., vaccines) alter one or more characteristics of the innate immune system. The present disclosure provides certain such compositions, including, for example, compositions that comprise RNA construct(s) encoding viral (e.g., HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus) protein(s) or fragments or epitopes thereof, as described herein.

Separately, in some embodiments, the present disclosure provides particular pharmaceutical composition (e.g., immunogenic composition, e.g., vaccine) formats including, for example, RNA pharmaceutical compositions (e.g., immunogenic compositions, e.g., vaccines) comprising particular elements and/or sequences useful for vaccination.

The present disclosure provides a variety of insights and technologies related to such viral (e.g., HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus) antigen constructs and vaccine (e.g., RNA vaccine) compositions.

As described herein, in many embodiments, provided compositions (e.g. pharmaceutical compositions, e.g., immunogenic compositions, e.g., vaccines) include an RNA active encoding one or more viral (e.g., HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus) antigens or fragments or epitopes thereof; in some embodiments such RNA active is a modified RNA format in that its uridine residues are substituted with uridine analog(s) such as pseudouridine; alternatively or additionally, in some embodiments, such RNA active includes particular elements (e.g., cap, 5′UTR, 3′UTR, polyA tail, etc) and/or characteristics (e.g., codon optimization) identified, selected, characterized, and/or demonstrated to achieve significant (e.g., elevated) translatability (e.g., in vitro) and/or expression (i.e., in a subject to whom it has been administered) of encoded protein(s). Still further alternatively or additionally, in some embodiments, such RNA active includes particular elements and/or characteristics identified, selected, characterized, and/or demonstrated to achieve significant RNA stability and/or efficient manufacturing, particularly at large scale (e.g., 0.1-10 g, 10-500 g, 500 g-1 kg, 750 g-1.5 kg; those skilled in the art will appreciate that different products may be manufactured at different scales, e.g., depending on patient population size). In some embodiments, such RNA manufacturing scale may be within a range of about 0.01 g/hr RNA to about 1 g/hr RNA, 1 g/hr RNA to about 100 g/hr RNA, about 1 g RNA/hr to about 20 g RNA/hr, or about 100 g RNA/hr to about 10,000 g RNA/hr. In some embodiments, such RNA manufacturing scale may be tens or hundreds of milligrams to tens or hundreds of grams (or more) of RNA per batch. In some embodiments, such RNA manufacturing scale may allow a batch size within a range of about 0.01 g to about 500 g RNA, about 0.01 g to about 10 g RNA, about 1 g to about 10 g RNA, about 10 g to about 500 g RNA, about 10 g to about 300 g RNA, about 10 g to about 200 g RNA or about 30 g to about 60 g RNA.

Still further, in many embodiments, provided compositions (e.g., pharmaceutical compositions, e.g., immunogenic compositions, e.g., vaccines) that include an RNA active are prepared, formulated, and/or utilized in particular LNP compositions, as described herein.

Among other things, the present disclosure provides technologies for rapid development of a pharmaceutical composition (e.g., immunogenic composition, e.g., viral vaccine) for delivering particular viral (e.g., HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus) antigen constructs to a subject.

Additionally, the present disclosure provides, for example, nucleic acid constructs encoding viral (e.g., HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus) antigens as described herein, expressed viral (e.g., HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus) proteins, and various methods of production and/or use relating thereto, as well as compositions developed therewith and methods relating thereto.

For example, the present disclosure provides technologies for preventing, characterizing, treating, and/or monitoring viral (e.g., HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus) outbreaks and/or infections including, as noted, various nucleic acid constructs and encoded proteins, as well as agents (e.g., antibodies) that bind to such proteins, and compositions that comprise and/or deliver them.

In some aspects, provided herein are technologies (e.g., compositions and methods) for augmenting, inducing, promoting, enhancing and/or improving an immune response against viral (e.g., HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus) or a component thereof (e.g., a protein or portion thereof). In some embodiments, technologies provided herein are designed to augment, induce, promote, enhance and/or improve immunological memory against viruses (e.g., HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus) or a component thereof(e.g., a protein or portion thereof). In some embodiments, technologies described herein are designed to act as an immunological boost to a primary vaccine, such as a vaccine directed to an epitope and/or epitopes of a virus (e.g., HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus). In some embodiments, compositions of the present disclosure comprise one or more polynucleotide constructs (e.g., one or more string constructs) that encode one or more epitopes from a virus (e.g., HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus). In some embodiments, the present disclosure provides vaccines or other compositions comprising nucleic acids encoding such viral (e.g., HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus) epitopes; those skilled in the art will appreciate from context when reference to a particular polynucleotide (e.g., a DNA or RNA) as “encoding” such epitopes in fact is referencing a coding strand or its complement.

### BRIEF DESCRIPTION OF THE DRAWING

FIG. 1 depicts exemplary sequence analyses performed on amino acid sequences encoded by ORF0 of the VZV genome. FIG. 1A shows an identification of exemplary predicted epitopes with respect to positions along an ORF0 consensus sequence; FIG. 1B depicts conservation scores determined for amino acids located at positions along an ORF0 consensus sequence.

FIG. 2 depicts exemplary sequence analyses performed on amino acid sequences encoded by ORF10 of the VZV genome. FIG. 2A shows an identification of exemplary predicted epitopes with respect to positions along an ORF10 consensus sequence; FIG. 2B depicts conservation scores determined for amino acids located at positions along an ORF10 consensus sequence.

FIG. 3A depicts exemplary sequence analyses performed on amino acid sequences encoded by ORF11 of the VZV genome. FIG. 3A shows an identification of exemplary predicted epitopes with respect to positions along an ORF11 consensus sequence; FIG. 3B depicts conservation scores determined for amino acids located at positions along an ORF11 consensus sequence.

FIG. 4 depicts exemplary sequence analyses performed on amino acid sequences encoded by ORF12 of the VZV genome. FIG. 4A shows an identification of exemplary predicted epitopes with respect to positions along an ORF12 consensus sequence; FIG. 4B depicts conservation scores determined for amino acids located at positions along an ORF12 consensus sequence.

FIG. 5 depicts exemplary sequence analyses performed on amino acid sequences encoded by ORF13 of the VZV genome. FIG. 5A shows an identification of exemplary predicted epitopes with respect to positions along an ORF13 consensus sequence; FIG. 5B depicts conservation scores determined for amino acids located at positions along an ORF13 consensus sequence.

FIG. 6 depicts exemplary sequence analyses performed on amino acid sequences encoded by ORF14 of the VZV genome. FIG. 6A shows an identification of exemplary predicted epitopes with respect to positions along an ORF14 consensus sequence; FIG. 6B depicts conservation scores determined for amino acids located at positions along an ORF14 consensus sequence.

FIG. 7 depicts exemplary sequence analyses performed on amino acid sequences encoded by ORF15 of the VZV genome. FIG. 7A shows an identification of exemplary predicted epitopes with respect to positions along an ORF15 consensus sequence; FIG. 7B depicts conservation scores determined for amino acids located at positions along an ORF15 consensus sequence.

FIG. 8 depicts exemplary sequence analyses performed on amino acid sequences encoded by ORF16 of the VZV genome. FIG. 8A shows an identification of exemplary predicted epitopes with respect to positions along an ORF16 consensus sequence; FIG. 8B depicts conservation scores determined for amino acids located at positions along an ORF16 consensus sequence.

FIG. 9 depicts exemplary sequence analyses performed on amino acid sequences encoded by ORF17 of the VZV genome. FIG. 9A shows an identification of exemplary predicted epitopes with respect to positions along an ORF17 consensus sequence; FIG. 9B depicts conservation scores determined for amino acids located at positions along an ORF17 consensus sequence.

FIG. 10 depicts exemplary sequence analyses performed on amino acid sequences encoded by ORF18 of the VZV genome. FIG. 10A shows an identification of exemplary predicted epitopes with respect to positions along an ORF18 consensus sequence; FIG. 10B depicts conservation scores determined for amino acids located at positions along an ORF18 consensus sequence.

FIG. 11 depicts exemplary sequence analyses performed on amino acid sequences encoded by ORF19 of the VZV genome. FIG. 11A shows an identification of exemplary predicted epitopes with respect to positions along an ORF19 consensus sequence; FIG. 11B depicts conservation scores determined for amino acids located at positions along an ORF19 consensus sequence.

FIG. 12 depicts exemplary sequence analyses performed on amino acid sequences encoded by ORF1 of the VZV genome. FIG. 12A shows an identification of exemplary predicted epitopes with respect to positions along an ORF1 consensus sequence; FIG. 12B depicts conservation scores determined for amino acids located at positions along an ORF1 consensus sequence.

FIG. 13 depicts exemplary sequence analyses performed on amino acid sequences encoded by ORF20 of the VZV genome. FIG. 13A shows an identification of exemplary predicted epitopes with respect to positions along an ORF20 consensus sequence; FIG. 13B depicts conservation scores determined for amino acids located at positions along an ORF20 consensus sequence.

FIG. 14 depicts exemplary sequence analyses performed on amino acid sequences encoded by ORF21 of the VZV genome. FIG. 14A shows an identification of exemplary predicted epitopes with respect to positions along an ORF21 consensus sequence; FIG. 14B depicts conservation scores determined for amino acids located at positions along an ORF21 consensus sequence.

FIG. 15 depicts exemplary sequence analyses performed on amino acid sequences encoded by ORF22 of the VZV genome. FIG. 15A shows an identification of exemplary predicted epitopes with respect to positions along an ORF22 consensus sequence; FIG. 15B depicts conservation scores determined for amino acids located at positions along an ORF22 consensus sequence.

FIG. 16 depicts exemplary sequence analyses performed on amino acid sequences encoded by ORF23 of the VZV genome. FIG. 16A shows an identification of exemplary predicted epitopes with respect to positions along an ORF23 consensus sequence; FIG. 16B depicts conservation scores determined for amino acids located at positions along an ORF23 consensus sequence.

FIG. 17 depicts exemplary sequence analyses performed on amino acid sequences encoded by ORF24 of the VZV genome. FIG. 17A shows an identification of exemplary predicted epitopes with respect to positions along an ORF24 consensus sequence; FIG. 17B depicts conservation scores determined for amino acids located at positions along an ORF24 consensus sequence.

FIG. 18 depicts exemplary sequence analyses performed on amino acid sequences encoded by ORF25 of the VZV genome. FIG. 18A shows an identification of exemplary predicted epitopes with respect to positions along an ORF25 consensus sequence; FIG. 18B depicts conservation scores determined for amino acids located at positions along an ORF25 consensus sequence.

FIG. 19 depicts exemplary sequence analyses performed on amino acid sequences encoded by ORF26 of the VZV genome. FIG. 19A shows an identification of exemplary predicted epitopes with respect to positions along an ORF26 consensus sequence; FIG. 19B depicts conservation scores determined for amino acids located at positions along an ORF26 consensus sequence.

FIG. 20 depicts exemplary sequence analyses performed on amino acid sequences encoded by ORF27 of the VZV genome. FIG. 20A shows an identification of exemplary predicted epitopes with respect to positions along an ORF27 consensus sequence; FIG. 20B depicts conservation scores determined for amino acids located at positions along an ORF27 consensus sequence.

FIG. 21 depicts exemplary sequence analyses performed on amino acid sequences encoded by ORF28 of the VZV genome. FIG. 21A shows an identification of exemplary predicted epitopes with respect to positions along an ORF28 consensus sequence; FIG. 21B depicts conservation scores determined for amino acids located at positions along an ORF28 consensus sequence.

FIG. 22 depicts exemplary sequence analyses performed on amino acid sequences encoded by ORF29 of the VZV genome. FIG. 22A shows an identification of exemplary predicted epitopes with respect to positions along an ORF29 consensus sequence; FIG. 22B depicts conservation scores determined for amino acids located at positions along an ORF29 consensus sequence.

FIG. 23 depicts exemplary sequence analyses performed on amino acid sequences encoded by ORF2 of the VZV genome. FIG. 23A shows an identification of exemplary predicted epitopes with respect to positions along an ORF2 consensus sequence; FIG. 23B depicts conservation scores determined for amino acids located at positions along an ORF2 consensus sequence.

FIG. 24 depicts exemplary sequence analyses performed on amino acid sequences encoded by ORF30 of the VZV genome. FIG. 24A shows an identification of exemplary predicted epitopes with respect to positions along an ORF30 consensus sequence; FIG. 24B depicts conservation scores determined for amino acids located at positions along an ORF30 consensus sequence.

FIG. 25 depicts exemplary sequence analyses performed on amino acid sequences encoded by ORF31 of the VZV genome. FIG. 25A shows an identification of exemplary predicted epitopes with respect to positions along an ORF31 consensus sequence; FIG. 25B depicts conservation scores determined for amino acids located at positions along an ORF31 consensus sequence.

FIG. 26 depicts exemplary sequence analyses performed on amino acid sequences encoded by ORF32 of the VZV genome. FIG. 26A shows an identification of exemplary predicted epitopes with respect to positions along an ORF32 consensus sequence; FIG. 26B depicts conservation scores determined for amino acids located at positions along an ORF32 consensus sequence.

FIG. 27 depicts exemplary sequence analyses performed on amino acid sequences encoded by ORF33 of the VZV genome. FIG. 27A shows an identification of exemplary predicted epitopes with respect to positions along an ORF33 consensus sequence; FIG. 27B depicts conservation scores determined for amino acids located at positions along an ORF33 consensus sequence.

FIG. 28 depicts exemplary sequence analyses performed on amino acid sequences encoded by ORF34 of the VZV genome. FIG. 28A shows an identification of exemplary predicted epitopes with respect to positions along an ORF34 consensus sequence; FIG. 28B depicts conservation scores determined for amino acids located at positions along an ORF34 consensus sequence.

FIG. 29 depicts exemplary sequence analyses performed on amino acid sequences encoded by ORF35 of the VZV genome. FIG. 29A shows an identification of exemplary predicted epitopes with respect to positions along an ORF35 consensus sequence; FIG. 29B depicts conservation scores determined for amino acids located at positions along an ORF35 consensus sequence.

FIG. 30 depicts exemplary sequence analyses performed on amino acid sequences encoded by ORF36 of the VZV genome. FIG. 30A shows an identification of exemplary predicted epitopes with respect to positions along an ORF36 consensus sequence; FIG. 30B depicts conservation scores determined for amino acids located at positions along an ORF36 consensus sequence.

FIG. 31 depicts exemplary sequence analyses performed on amino acid sequences encoded by ORF37 of the VZV genome. FIG. 31A shows an identification of exemplary predicted epitopes with respect to positions along an ORF37 consensus sequence; FIG. 31B depicts conservation scores determined for amino acids located at positions along an ORF37 consensus sequence.

FIG. 32 depicts exemplary sequence analyses performed on amino acid sequences encoded by ORF38 of the VZV genome. FIG. 32A shows an identification of exemplary predicted epitopes with respect to positions along an ORF38 consensus sequence; FIG. 32B depicts conservation scores determined for amino acids located at positions along an ORF38 consensus sequence.

FIG. 33 depicts exemplary sequence analyses performed on amino acid sequences encoded by ORF39 of the VZV genome. FIG. 33A shows an identification of exemplary predicted epitopes with respect to positions along an ORF39 consensus sequence; FIG. 33B depicts conservation scores determined for amino acids located at positions along an ORF39 consensus sequence.

FIG. 34 depicts exemplary sequence analyses performed on amino acid sequences encoded by ORF3 of the VZV genome. FIG. 34A shows an identification of exemplary predicted epitopes with respect to positions along an ORF3 consensus sequence; FIG. 34B depicts conservation scores determined for amino acids located at positions along an ORF3 consensus sequence.

FIG. 35 depicts exemplary sequence analyses performed on amino acid sequences encoded by ORF40 of the VZV genome. FIG. 35A shows an identification of exemplary predicted epitopes with respect to positions along an ORF40 consensus sequence; FIG. 35B depicts conservation scores determined for amino acids located at positions along an ORF40 consensus sequence.

FIG. 36 depicts exemplary sequence analyses performed on amino acid sequences encoded by ORF41 of the VZV genome. FIG. 36A shows an identification of exemplary predicted epitopes with respect to positions along an ORF41 consensus sequence; FIG. 36B depicts conservation scores determined for amino acids located at positions along an ORF41 consensus sequence.

FIG. 37 depicts exemplary sequence analyses performed on amino acid sequences encoded by ORF42 of the VZV genome. FIG. 37A shows an identification of exemplary predicted epitopes with respect to positions along an ORF42 consensus sequence; FIG. 37B depicts conservation scores determined for amino acids located at positions along an ORF42 consensus sequence.

FIG. 38 depicts exemplary sequence analyses performed on amino acid sequences encoded by ORF43 of the VZV genome. FIG. 38A shows an identification of exemplary predicted epitopes with respect to positions along an ORF43 consensus sequence; FIG. 38B depicts conservation scores determined for amino acids located at positions along an ORF43 consensus sequence.

FIG. 39 depicts exemplary sequence analyses performed on amino acid sequences encoded by ORF44 of the VZV genome. FIG. 39A shows an identification of exemplary predicted epitopes with respect to positions along an ORF44 consensus sequence; FIG. 39B depicts conservation scores determined for amino acids located at positions along an ORF44 consensus sequence.

FIG. 40 depicts exemplary sequence analyses performed on amino acid sequences encoded by ORF45 of the VZV genome. FIG. 40A shows an identification of exemplary predicted epitopes with respect to positions along an ORF45 consensus sequence; FIG. 40B depicts conservation scores determined for amino acids located at positions along an ORF45 consensus sequence.

FIG. 41 depicts exemplary sequence analyses performed on amino acid sequences encoded by ORF46 of the VZV genome. FIG. 41A shows an identification of exemplary predicted epitopes with respect to positions along an ORF46 consensus sequence; FIG. 41B depicts conservation scores determined for amino acids located at positions along an ORF46 consensus sequence.

FIG. 42 depicts exemplary sequence analyses performed on amino acid sequences encoded by ORF47 of the VZV genome. FIG. 42A shows an identification of exemplary predicted epitopes with respect to positions along an ORF47 consensus sequence; FIG. 42B depicts conservation scores determined for amino acids located at positions along an ORF47 consensus sequence.

FIG. 43 depicts exemplary sequence analyses performed on amino acid sequences encoded by ORF48 of the VZV genome. FIG. 43A shows an identification of exemplary predicted epitopes with respect to positions along an ORF48 consensus sequence; FIG. 43B depicts conservation scores determined for amino acids located at positions along an ORF48 consensus sequence.

FIG. 44 depicts exemplary sequence analyses performed on amino acid sequences encoded by ORF49 of the VZV genome. FIG. 44A shows an identification of exemplary predicted epitopes with respect to positions along an ORF49 consensus sequence; FIG. 44B depicts conservation scores determined for amino acids located at positions along an ORF49 consensus sequence.

FIG. 45 depicts exemplary sequence analyses performed on amino acid sequences encoded by ORF4 of the VZV genome. FIG. 45A shows an identification of exemplary predicted epitopes with respect to positions along an ORF4 consensus sequence; FIG. 45B depicts conservation scores determined for amino acids located at positions along an ORF4 consensus sequence.

FIG. 46 depicts exemplary sequence analyses performed on amino acid sequences encoded by ORF50 of the VZV genome. FIG. 46A shows an identification of exemplary predicted epitopes with respect to positions along an ORF50 consensus sequence; FIG. 46B depicts conservation scores determined for amino acids located at positions along an ORF50 consensus sequence.

FIG. 47 depicts exemplary sequence analyses performed on amino acid sequences encoded by ORF51 of the VZV genome. FIG. 47A shows an identification of exemplary predicted epitopes with respect to positions along an ORF51 consensus sequence; FIG. 47B depicts conservation scores determined for amino acids located at positions along an ORF51 consensus sequence.

FIG. 48 depicts exemplary sequence analyses performed on amino acid sequences encoded by ORF52 of the VZV genome. FIG. 48A shows an identification of exemplary predicted epitopes with respect to positions along an ORF52 consensus sequence; FIG. 48B depicts conservation scores determined for amino acids located at positions along an ORF52 consensus sequence.

FIG. 49 depicts exemplary sequence analyses performed on amino acid sequences encoded by ORF53 of the VZV genome. FIG. 49A shows an identification of exemplary predicted epitopes with respect to positions along an ORF53 consensus sequence; FIG. 49B depicts conservation scores determined for amino acids located at positions along an ORF53 consensus sequence.

FIG. 50 depicts exemplary sequence analyses performed on amino acid sequences encoded by ORF54 of the VZV genome. FIG. 50A shows an identification of exemplary predicted epitopes with respect to positions along an ORF54 consensus sequence; FIG. 50B depicts conservation scores determined for amino acids located at positions along an ORF54 consensus sequence.

FIG. 51 depicts exemplary sequence analyses performed on amino acid sequences encoded by ORF55 of the VZV genome. FIG. 51A shows an identification of exemplary predicted epitopes with respect to positions along an ORF55 consensus sequence; FIG. 51B depicts conservation scores determined for amino acids located at positions along an ORF55 consensus sequence.

FIG. 52 depicts exemplary sequence analyses performed on amino acid sequences encoded by ORF56 of the VZV genome. FIG. 52A shows an identification of exemplary predicted epitopes with respect to positions along an ORF56 consensus sequence; FIG. 52B depicts conservation scores determined for amino acids located at positions along an ORF56 consensus sequence.

FIG. 53 depicts exemplary sequence analyses performed on amino acid sequences encoded by ORF57 of the VZV genome. FIG. 53A shows an identification of exemplary predicted epitopes with respect to positions along an ORF57 consensus sequence; FIG. 53B depicts conservation scores determined for amino acids located at positions along an ORF57 consensus sequence.

FIG. 54 depicts exemplary sequence analyses performed on amino acid sequences encoded by ORF58 of the VZV genome. FIG. 54A shows an identification of exemplary predicted epitopes with respect to positions along an ORF58 consensus sequence; FIG. 54B depicts conservation scores determined for amino acids located at positions along an ORF58 consensus sequence.

FIG. 55 depicts exemplary sequence analyses performed on amino acid sequences encoded by ORF59 of the VZV genome. FIG. 55A shows an identification of exemplary predicted epitopes with respect to positions along an ORF59 consensus sequence; FIG. 55B depicts conservation scores determined for amino acids located at positions along an ORF59 consensus sequence.

FIG. 56 depicts exemplary sequence analyses performed on amino acid sequences encoded by ORF5 of the VZV genome. FIG. 56A shows an identification of exemplary predicted epitopes with respect to positions along an ORF5 consensus sequence; FIG. 56B depicts conservation scores determined for amino acids located at positions along an ORF5 consensus sequence.

FIG. 57 depicts exemplary sequence analyses performed on amino acid sequences encoded by ORF60 of the VZV genome. FIG. 57A shows an identification of exemplary predicted epitopes with respect to positions along an ORF60 consensus sequence; FIG. 57B depicts conservation scores determined for amino acids located at positions along an ORF60 consensus sequence.

FIG. 58 depicts exemplary sequence analyses performed on amino acid sequences encoded by ORF61 of the VZV genome. FIG. 58A shows an identification of exemplary predicted epitopes with respect to positions along an ORF61 consensus sequence; FIG. 58B depicts conservation scores determined for amino acids located at positions along an ORF61 consensus sequence.

FIG. 59 depicts exemplary sequence analyses performed on amino acid sequences encoded by ORF62 of the VZV genome. FIG. 59A shows an identification of exemplary predicted epitopes with respect to positions along an ORF62 consensus sequence; FIG. 59B depicts conservation scores determined for amino acids located at positions along an ORF62 consensus sequence.

FIG. 60 depicts exemplary sequence analyses performed on amino acid sequences encoded by ORF63 of the VZV genome. FIG. 60A shows an identification of exemplary predicted epitopes with respect to positions along an ORF63 consensus sequence; FIG. 60B depicts conservation scores determined for amino acids located at positions along an ORF63 consensus sequence.

FIG. 61 depicts exemplary sequence analyses performed on amino acid sequences encoded by ORF64 of the VZV genome. FIG. 61A shows an identification of exemplary predicted epitopes with respect to positions along an ORF64 consensus sequence; FIG. 61B depicts conservation scores determined for amino acids located at positions along an ORF64 consensus sequence.

FIG. 62 depicts exemplary sequence analyses performed on amino acid sequences encoded by ORF65 of the VZV genome. FIG. 62A shows an identification of exemplary predicted epitopes with respect to positions along an ORF65 consensus sequence; FIG. 62B depicts conservation scores determined for amino acids located at positions along an ORF65 consensus sequence.

FIG. 63 depicts exemplary sequence analyses performed on amino acid sequences encoded by ORF66 of the VZV genome. FIG. 63A shows an identification of exemplary predicted epitopes with respect to positions along an ORF66 consensus sequence; FIG. 63B depicts conservation scores determined for amino acids located at positions along an ORF66 consensus sequence.

FIG. 64 depicts exemplary sequence analyses performed on amino acid sequences encoded by ORF67 of the VZV genome. FIG. 64A shows an identification of exemplary predicted epitopes with respect to positions along an ORF67 consensus sequence; FIG. 64B depicts conservation scores determined for amino acids located at positions along an ORF67 consensus sequence.

FIG. 65 depicts exemplary sequence analyses performed on amino acid sequences encoded by ORF68 of the VZV genome. FIG. 65A shows an identification of exemplary predicted epitopes with respect to positions along an ORF68 consensus sequence; FIG. 65B depicts conservation scores determined for amino acids located at positions along an ORF68 consensus sequence.

FIG. 66 depicts exemplary sequence analyses performed on amino acid sequences encoded by ORF69 of the VZV genome. FIG. 66A shows an identification of exemplary predicted epitopes with respect to positions along an ORF69 consensus sequence; FIG. 66B depicts conservation scores determined for amino acids located at positions along an ORF69 consensus sequence.

FIG. 67 depicts exemplary sequence analyses performed on amino acid sequences encoded by ORF6 of the VZV genome. FIG. 67A shows an identification of exemplary predicted epitopes with respect to positions along an ORF6 consensus sequence; FIG. 67B depicts conservation scores determined for amino acids located at positions along an ORF6 consensus sequence.

FIG. 68 depicts exemplary sequence analyses performed on amino acid sequences encoded by ORF70 of the VZV genome. FIG. 68A shows an identification of exemplary predicted epitopes with respect to positions along an ORF70 consensus sequence; FIG. 68B depicts conservation scores determined for amino acids located at positions along an ORF70 consensus sequence.

FIG. 69 depicts exemplary sequence analyses performed on amino acid sequences encoded by ORF71 of the VZV genome. FIG. 69A shows an identification of exemplary predicted epitopes with respect to positions along an ORF71 consensus sequence; FIG. 69B depicts conservation scores determined for amino acids located at positions along an ORF71 consensus sequence.

FIG. 70 depicts exemplary sequence analyses performed on amino acid sequences encoded by ORF7 of the VZV genome. FIG. 70A shows an identification of exemplary predicted epitopes with respect to positions along an ORF7 consensus sequence; FIG. 70B depicts conservation scores determined for amino acids located at positions along an ORF7 consensus sequence.

FIG. 71 depicts exemplary sequence analyses performed on amino acid sequences encoded by ORF8 of the VZV genome. FIG. 71A shows an identification of exemplary predicted epitopes with respect to positions along an ORF8 consensus sequence; FIG. 71B depicts conservation scores determined for amino acids located at positions along an ORF8 consensus sequence.

FIG. 72 depicts exemplary sequence analyses performed on amino acid sequences encoded by ORF9A of the VZV genome. FIG. 72A shows an identification of exemplary predicted epitopes with respect to positions along an ORF9A consensus sequence; FIG. 72B depicts conservation scores determined for amino acids located at positions along an ORF9A consensus sequence.

FIG. 73 depicts exemplary sequence analyses performed on amino acid sequences encoded by ORF9 of the VZV genome. FIG. 73A shows an identification of exemplary predicted epitopes with respect to positions along an ORF9 consensus sequence; FIG. 73B depicts conservation scores determined for amino acids located at positions along an ORF9 consensus sequence.

FIG. 74 has been modified from Zerboni, L., et al., “Molecular mechanisms of varicella zoster virus pathogenesis,” Nat Rev Microbiol., 12(3):197-210 (March 2014), which is incorporated herein by reference in its entirety. FIG. 74 depicts a model of the VZV life cycle.

FIG. 75 has been modified from Gershon, A. A., et al., “Varicella zoster virus infection,” Nat Rev Dis Primers, 2015 Jul. 2; 1:15016 (July 2015), which is incorporated herein by reference in its entirety. FIG. 75 depicts a schematic showing different phases of VZV infection.

FIG. 76 has been modified from Gershon (2015), which is incorporated herein by reference in its entirety. FIG. 76 depicts a schematic of lytic VZV infection.

FIG. 77 has been modified from Gershon (2015), which is incorporated herein by reference in its entirety. FIG. 77 depicts a schematic of latent VZV infection.

FIG. 78 presents certain exemplary VZV envelope proteins whose sequences may be utilized as and/or included in antigen(s) in accordance with the present disclosure.

FIG. 79 presents an optional immunization protocol for mouse studies.

FIG. 80 presents and exemplary workflow for identification, selection and/or characterization of antigens (e.g., VZV proteins, including particular variants, and/or epitopes thereof, in particular T cell epitopes) for use in accordance with the present disclosure.

FIG. 81 includes phylogenetic trees of VZV strains, which were originally presented in Peters, G. A., “A Full-Genome Phylogenetic Analysis of Varicella-Zoster Virus Reveals a Novel Origin of Replication-Based Genotyping Scheme and Evidence of Recombination between Major Circulating Clades,” Journal of Virology, 80(19): 9850-9860 (2006), which is incorporated herein by reference in its entirety. FIG. 81A includes a phylogenetic tree of VZV strains based on full-genome sequence, FIG. 81B includes a phylogenetic tree of VZV strains based on aligned sequences of five glycoprotein genes and IE62 (B), and FIG. 81C includes a phylogenetic tree of VZV strains based on origin of replication region.

FIG. 82 includes the sequence of an exemplary VZV gE protein fragment antigen. U.S. Pat. No. 7,939,084, which is incorporated herein by reference in its entirety.

FIG. 83 depicts exemplary sequence analyses performed on amino acid sequences encoded by IRS1 of the CMV genome. FIG. 83A shows an identification of exemplary predicted epitopes with respect to positions along an IRS1 consensus sequence; FIG. 83B depicts conservation scores determined for amino acids located at positions along an IRS1 consensus sequence.

FIG. 84 depicts exemplary sequence analyses performed on amino acid sequences encoded by RL10 of the CMV genome. FIG. 84A shows an identification of exemplary predicted epitopes with respect to positions along an RL10 consensus sequence; FIG. 84B depicts conservation scores determined for amino acids located at positions along an RL10 consensus sequence.

FIG. 85 depicts exemplary sequence analyses performed on amino acid sequences encoded by RL11 of the CMV genome. FIG. 85A shows an identification of exemplary predicted epitopes with respect to positions along an RL11 consensus sequence; FIG. 85B depicts conservation scores determined for amino acids located at positions along an RL11 consensus sequence.

FIG. 86 depicts exemplary sequence analyses performed on amino acid sequences encoded by RL12 of the CMV genome. FIG. 86A shows an identification of exemplary predicted epitopes with respect to positions along an RL12 consensus sequence; FIG. 86B depicts conservation scores determined for amino acids located at positions along an RL12 consensus sequence.

FIG. 87 depicts exemplary sequence analyses performed on amino acid sequences encoded by RL13 of the CMV genome. FIG. 87A shows an identification of exemplary predicted epitopes with respect to positions along an RL13 consensus sequence; FIG. 87B depicts conservation scores determined for amino acids located at positions along an RL13 consensus sequence.

FIG. 88 depicts exemplary sequence analyses performed on amino acid sequences encoded by RL1 of the CMV genome. FIG. 88A shows an identification of exemplary predicted epitopes with respect to positions along an RL1 consensus sequence; FIG. 88B depicts conservation scores determined for amino acids located at positions along an RL1 consensus sequence.

FIG. 89 depicts exemplary sequence analyses performed on amino acid sequences encoded by RL2 of the CMV genome. FIG. 89A shows an identification of exemplary predicted epitopes with respect to positions along an RL2 consensus sequence; FIG. 89B depicts conservation scores determined for amino acids located at positions along an RL2 consensus sequence.

FIG. 90 depicts exemplary sequence analyses performed on amino acid sequences encoded by RL5A of the CMV genome. FIG. 90A shows an identification of exemplary predicted epitopes with respect to positions along an RL5A consensus sequence; FIG. 90B depicts conservation scores determined for amino acids located at positions along an RL5A consensus sequence.

FIG. 91 depicts exemplary sequence analyses performed on amino acid sequences encoded by RL6 of the CMV genome. FIG. 91A shows an identification of exemplary predicted epitopes with respect to positions along an RL6 consensus sequence; FIG. 91B depicts conservation scores determined for amino acids located at positions along an RL6 consensus sequence.

FIG. 92 depicts exemplary sequence analyses performed on amino acid sequences encoded by RL8A of the CMV genome. FIG. 92A shows an identification of exemplary predicted epitopes with respect to positions along an RL8A consensus sequence; FIG. 92B depicts conservation scores determined for amino acids located at positions along an RL8A consensus sequence.

FIG. 93 depicts exemplary sequence analyses performed on amino acid sequences encoded by RL9A of the CMV genome. FIG. 93A shows an identification of exemplary predicted epitopes with respect to positions along an RL9A consensus sequence; FIG. 93B depicts conservation scores determined for amino acids located at positions along an RL9A consensus sequence.

FIG. 94 depicts exemplary sequence analyses performed on amino acid sequences encoded by TRS1 of the CMV genome. FIG. 94A shows an identification of exemplary predicted epitopes with respect to positions along a TRS1 consensus sequence; FIG. 94B depicts conservation scores determined for amino acids located at positions along a TRS1 consensus sequence.

FIG. 95 depicts exemplary sequence analyses performed on amino acid sequences encoded by UL100 of the CMV genome. FIG. 95A shows an identification of exemplary predicted epitopes with respect to positions along a UL100 consensus sequence; FIG. 95B depicts conservation scores determined for amino acids located at positions along a UL100 consensus sequence.

FIG. 96 depicts exemplary sequence analyses performed on amino acid sequences encoded by UL102 of the CMV genome. FIG. 96A shows an identification of exemplary predicted epitopes with respect to positions along a UL102 consensus sequence; FIG. 96B depicts conservation scores determined for amino acids located at positions along a UL102 consensus sequence.

FIG. 97 depicts exemplary sequence analyses performed on amino acid sequences encoded by UL103 of the CMV genome. FIG. 97A shows an identification of exemplary predicted epitopes with respect to positions along a UL103 consensus sequence; FIG. 97B depicts conservation scores determined for amino acids located at positions along a UL103 consensus sequence.

FIG. 98 depicts exemplary sequence analyses performed on amino acid sequences encoded by UL104 of the CMV genome. FIG. 98A shows an identification of exemplary predicted epitopes with respect to positions along a UL104 consensus sequence; FIG. 98B depicts conservation scores determined for amino acids located at positions along a UL104 consensus sequence.

FIG. 99 depicts exemplary sequence analyses performed on amino acid sequences encoded by UL105 of the CMV genome. FIG. 99A shows an identification of exemplary predicted epitopes with respect to positions along a UL105 consensus sequence; FIG. 99B depicts conservation scores determined for amino acids located at positions along a UL105 consensus sequence.

FIG. 100 depicts exemplary sequence analyses performed on amino acid sequences encoded by UL10 of the CMV genome. FIG. 100A shows an identification of exemplary predicted epitopes with respect to positions along a UL10 consensus sequence; FIG. 100B depicts conservation scores determined for amino acids located at positions along a UL10 consensus sequence.

FIG. 101 depicts exemplary sequence analyses performed on amino acid sequences encoded by UL111A of the CMV genome. FIG. 101A shows an identification of exemplary predicted epitopes with respect to positions along a UL111A consensus sequence; FIG. 101B depicts conservation scores determined for amino acids located at positions along a UL111A consensus sequence.

FIG. 102 depicts exemplary sequence analyses performed on amino acid sequences encoded by UL112 of the CMV genome. FIG. 102A shows an identification of exemplary predicted epitopes with respect to positions along a UL112 consensus sequence; FIG. 102B depicts conservation scores determined for amino acids located at positions along a UL112 consensus sequence.

FIG. 103 depicts exemplary sequence analyses performed on amino acid sequences encoded by UL114 of the CMV genome. FIG. 103A shows an identification of exemplary predicted epitopes with respect to positions along a UL114 consensus sequence; FIG. 103B depicts conservation scores determined for amino acids located at positions along a UL114 consensus sequence.

FIG. 104 depicts exemplary sequence analyses performed on amino acid sequences encoded by UL115 of the CMV genome. FIG. 104A shows an identification of exemplary predicted epitopes with respect to positions along a UL115 consensus sequence; FIG. 104B depicts conservation scores determined for amino acids located at positions along a UL115 consensus sequence.

FIG. 105 depicts exemplary sequence analyses performed on amino acid sequences encoded by UL116 of the CMV genome. FIG. 105A shows an identification of exemplary predicted epitopes with respect to positions along a UL116 consensus sequence; FIG. 105B depicts conservation scores determined for amino acids located at positions along a UL116 consensus sequence.

FIG. 106 depicts exemplary sequence analyses performed on amino acid sequences encoded by UL117 of the CMV genome. FIG. 106A shows an identification of exemplary predicted epitopes with respect to positions along a UL117 consensus sequence; FIG. 106B depicts conservation scores determined for amino acids located at positions along a UL117 consensus sequence.

FIG. 107 depicts exemplary sequence analyses performed on amino acid sequences encoded by UL119 of the CMV genome. FIG. 107A shows an identification of exemplary predicted epitopes with respect to positions along a UL119 consensus sequence; FIG. 107B depicts conservation scores determined for amino acids located at positions along a UL119 consensus sequence.

FIG. 108 depicts exemplary sequence analyses performed on amino acid sequences encoded by UL11 of the CMV genome. FIG. 108A shows an identification of exemplary predicted epitopes with respect to positions along a UL11 consensus sequence; FIG. 108B depicts conservation scores determined for amino acids located at positions along a UL11 consensus sequence.

FIG. 109 depicts exemplary sequence analyses performed on amino acid sequences encoded by UL120 of the CMV genome. FIG. 109A shows an identification of exemplary predicted epitopes with respect to positions along a UL120 consensus sequence; FIG. 109B depicts conservation scores determined for amino acids located at positions along a UL120 consensus sequence.

FIG. 110 depicts exemplary sequence analyses performed on amino acid sequences encoded by UL121 of the CMV genome. FIG. 110A shows an identification of exemplary predicted epitopes with respect to positions along a UL121 consensus sequence; FIG. 110B depicts conservation scores determined for amino acids located at positions along a UL121 consensus sequence.

FIG. 111 depicts exemplary sequence analyses performed on amino acid sequences encoded by UL122 of the CMV genome. FIG. 111A shows an identification of exemplary predicted epitopes with respect to positions along a UL122 consensus sequence; FIG. 111B depicts conservation scores determined for amino acids located at positions along a UL122 consensus sequence.

FIG. 112 depicts exemplary sequence analyses performed on amino acid sequences encoded by UL123 of the CMV genome. FIG. 112A shows an identification of exemplary predicted epitopes with respect to positions along a UL123 consensus sequence; FIG. 112B depicts conservation scores determined for amino acids located at positions along a UL123 consensus sequence.

FIG. 113 depicts exemplary sequence analyses performed on amino acid sequences encoded by UL124 of the CMV genome. FIG. 113A shows an identification of exemplary predicted epitopes with respect to positions along a UL124 consensus sequence; FIG. 113B depicts conservation scores determined for amino acids located at positions along a UL124 consensus sequence.

FIG. 114 depicts exemplary sequence analyses performed on amino acid sequences encoded by UL128 of the CMV genome. FIG. 114A shows an identification of exemplary predicted epitopes with respect to positions along a UL128 consensus sequence; FIG. 114B depicts conservation scores determined for amino acids located at positions along a UL128 consensus sequence.

FIG. 115 depicts exemplary sequence analyses performed on amino acid sequences encoded by UL130 of the CMV genome. FIG. 115A shows an identification of exemplary predicted epitopes with respect to positions along a UL130 consensus sequence; FIG. 115B depicts conservation scores determined for amino acids located at positions along a UL130 consensus sequence.

FIG. 116 depicts exemplary sequence analyses performed on amino acid sequences encoded by UL131A of the CMV genome. FIG. 116A shows an identification of exemplary predicted epitopes with respect to positions along a UL131A consensus sequence; FIG. 116B depicts conservation scores determined for amino acids located at positions along a UL131A consensus sequence.

FIG. 117 depicts exemplary sequence analyses performed on amino acid sequences encoded by UL132 of the CMV genome. FIG. 117A shows an identification of exemplary predicted epitopes with respect to positions along a UL132 consensus sequence; FIG. 117B depicts conservation scores determined for amino acids located at positions along a UL132 consensus sequence.

FIG. 118 depicts exemplary sequence analyses performed on amino acid sequences encoded by UL133 of the CMV genome. FIG. 118A shows an identification of exemplary predicted epitopes with respect to positions along a UL133 consensus sequence; FIG. 118B depicts conservation scores determined for amino acids located at positions along a UL133 consensus sequence.

FIG. 119 depicts exemplary sequence analyses performed on amino acid sequences encoded by UL135 of the CMV genome. FIG. 119A shows an identification of exemplary predicted epitopes with respect to positions along a UL135 consensus sequence; FIG. 119B depicts conservation scores determined for amino acids located at positions along a UL135 consensus sequence.

FIG. 120 depicts exemplary sequence analyses performed on amino acid sequences encoded by UL136 of the CMV genome. FIG. 120A shows an identification of exemplary predicted epitopes with respect to positions along a UL136 consensus sequence; FIG. 120B depicts conservation scores determined for amino acids located at positions along a UL136 consensus sequence.

FIG. 121 depicts exemplary sequence analyses performed on amino acid sequences encoded by UL138 of the CMV genome. FIG. 121A shows an identification of exemplary predicted epitopes with respect to positions along a UL138 consensus sequence; FIG. 121B depicts conservation scores determined for amino acids located at positions along a UL138 consensus sequence.

FIG. 122 depicts exemplary sequence analyses performed on amino acid sequences encoded by UL139 of the CMV genome. FIG. 122A shows an identification of exemplary predicted epitopes with respect to positions along a UL139 consensus sequence; FIG. 122B depicts conservation scores determined for amino acids located at positions along a UL139 consensus sequence.

FIG. 123 depicts exemplary sequence analyses performed on amino acid sequences encoded by UL13 of the CMV genome. FIG. 123A shows an identification of exemplary predicted epitopes with respect to positions along a UL13 consensus sequence; FIG. 123B depicts conservation scores determined for amino acids located at positions along a UL13 consensus sequence.

FIG. 124 depicts exemplary sequence analyses performed on amino acid sequences encoded by UL140 of the CMV genome. FIG. 124A shows an identification of exemplary predicted epitopes with respect to positions along a UL140 consensus sequence; FIG. 124B depicts conservation scores determined for amino acids located at positions along a UL140 consensus sequence.

FIG. 125 depicts exemplary sequence analyses performed on amino acid sequences encoded by UL141 of the CMV genome. FIG. 125A shows an identification of exemplary predicted epitopes with respect to positions along a UL141 consensus sequence; FIG. 125B depicts conservation scores determined for amino acids located at positions along a UL141 consensus sequence.

FIG. 126 depicts exemplary sequence analyses performed on amino acid sequences encoded by UL142 of the CMV genome. FIG. 126A shows an identification of exemplary predicted epitopes with respect to positions along a UL142 consensus sequence; FIG. 126B depicts conservation scores determined for amino acids located at positions along a UL142 consensus sequence.

FIG. 127 depicts exemplary sequence analyses performed on amino acid sequences encoded by UL144 of the CMV genome. FIG. 127A shows an identification of exemplary predicted epitopes with respect to positions along a UL144 consensus sequence; FIG. 127B depicts conservation scores determined for amino acids located at positions along a UL144 consensus sequence.

FIG. 128 depicts exemplary sequence analyses performed on amino acid sequences encoded by UL145 of the CMV genome. FIG. 128A shows an identification of exemplary predicted epitopes with respect to positions along a UL145 consensus sequence; FIG. 128B depicts conservation scores determined for amino acids located at positions along a UL145 consensus sequence.

FIG. 129 depicts exemplary sequence analyses performed on amino acid sequences encoded by UL146 of the CMV genome. FIG. 129A shows an identification of exemplary predicted epitopes with respect to positions along a UL146 consensus sequence; FIG. 129B depicts conservation scores determined for amino acids located at positions along a UL146 UIsconsensus sequence.

FIG. 130 depicts exemplary sequence analyses performed on amino acid sequences encoded by UL147A of the CMV genome. FIG. 130A shows an identification of exemplary predicted epitopes with respect to positions along a UL147A consensus sequence; FIG. 130B depicts conservation scores determined for amino acids located at positions along a UL147A consensus sequence.

FIG. 131 depicts exemplary sequence analyses performed on amino acid sequences encoded by UL147 of the CMV genome. FIG. 131A shows an identification of exemplary predicted epitopes with respect to positions along a UL147 consensus sequence; FIG. 131B depicts conservation scores determined for amino acids located at positions along a UL147 consensus sequence.

FIG. 132 depicts exemplary sequence analyses performed on amino acid sequences encoded by UL148A of the CMV genome. FIG. 132A shows an identification of exemplary predicted epitopes with respect to positions along a UL148A consensus sequence; FIG. 132B depicts conservation scores determined for amino acids located at positions along a UL148A consensus sequence.

FIG. 133 depicts exemplary sequence analyses performed on amino acid sequences encoded by UL148B of the CMV genome. FIG. 133A shows an identification of exemplary predicted epitopes with respect to positions along a UL148B consensus sequence; FIG. 133B depicts conservation scores determined for amino acids located at positions along a UL148B consensus sequence.

FIG. 134 depicts exemplary sequence analyses performed on amino acid sequences encoded by UL148C of the CMV genome. FIG. 134A shows an identification of exemplary predicted epitopes with respect to positions along a UL148C consensus sequence; FIG. 134B depicts conservation scores determined for amino acids located at positions along a UL148C consensus sequence.

FIG. 135 depicts exemplary sequence analyses performed on amino acid sequences encoded by UL148D of the CMV genome. FIG. 135A shows an identification of exemplary predicted epitopes with respect to positions along a UL148D consensus sequence; FIG. 135B depicts conservation scores determined for amino acids located at positions along a UL148D consensus sequence.

FIG. 136 depicts exemplary sequence analyses performed on amino acid sequences encoded by UL148 of the CMV genome. FIG. 136A shows an identification of exemplary predicted epitopes with respect to positions along a UL148 consensus sequence; FIG. 136B depicts conservation scores determined for amino acids located at positions along a UL148 consensus sequence.

FIG. 137 depicts exemplary sequence analyses performed on amino acid sequences encoded by UL14 of the CMV genome. FIG. 137A shows an identification of exemplary predicted epitopes with respect to positions along a UL14 consensus sequence; FIG. 137B depicts conservation scores determined for amino acids located at positions along a UL14 consensus sequence.

FIG. 138 depicts exemplary sequence analyses performed on amino acid sequences encoded by UL150A of the CMV genome. FIG. 138A shows an identification of exemplary predicted epitopes with respect to positions along a UL150A consensus sequence; FIG. 138B depicts conservation scores determined for amino acids located at positions along a UL150A consensus sequence.

FIG. 139 depicts exemplary sequence analyses performed on amino acid sequences encoded by UL150 of the CMV genome. FIG. 139A shows an identification of exemplary predicted epitopes with respect to positions along a UL150 consensus sequence; FIG. 139B depicts conservation scores determined for amino acids located at positions along a UL150 consensus sequence.

FIG. 140 depicts exemplary sequence analyses performed on amino acid sequences encoded by UL15A of the CMV genome. FIG. 140A shows an identification of exemplary predicted epitopes with respect to positions along a UL15A consensus sequence; FIG. 140B depicts conservation scores determined for amino acids located at positions along a UL15A sequence.

FIG. 141 depicts exemplary sequence analyses performed on amino acid sequences encoded by UL16 of the CMV genome. FIG. 141A shows an identification of exemplary predicted epitopes with respect to positions along a UL16 consensus sequence; FIG. 141B depicts conservation scores determined for amino acids located at positions along a UL16 consensus sequence.

FIG. 142 depicts exemplary sequence analyses performed on amino acid sequences encoded by UL17 of the CMV genome. FIG. 142A shows an identification of exemplary predicted epitopes with respect to positions along a UL17 consensus sequence; FIG. 142B depicts conservation scores determined for amino acids located at positions along a UL17 consensus sequence.

FIG. 143 depicts exemplary sequence analyses performed on amino acid sequences encoded by UL18 of the CMV genome. FIG. 143A shows an identification of exemplary predicted epitopes with respect to positions along a UL18 consensus sequence; FIG. 143B depicts conservation scores determined for amino acids located at positions along a UL18 consensus sequence.

FIG. 144 depicts exemplary sequence analyses performed on amino acid sequences encoded by UL19 of the CMV genome. FIG. 144A shows an identification of exemplary predicted epitopes with respect to positions along a UL19 consensus sequence; FIG. 144B depicts conservation scores determined for amino acids located at positions along a UL19 consensus sequence.

FIG. 145 depicts exemplary sequence analyses performed on amino acid sequences encoded by UL1 of the CMV genome. FIG. 145A shows an identification of exemplary predicted epitopes with respect to positions along a UL1 consensus sequence; FIG. 145B depicts conservation scores determined for amino acids located at positions along a UL1 consensus sequence.

FIG. 146 depicts exemplary sequence analyses performed on amino acid sequences encoded by UL20 of the CMV genome. FIG. 146A shows an identification of exemplary predicted epitopes with respect to positions along a UL20 consensus sequence; FIG. 146B depicts conservation scores determined for amino acids located at positions along a UL20 consensus sequence.

FIG. 147 depicts exemplary sequence analyses performed on amino acid sequences encoded by UL21A of the CMV genome. FIG. 147A shows an identification of exemplary predicted epitopes with respect to positions along a UL21A consensus sequence; FIG. 147B depicts conservation scores determined for amino acids located at positions along a UL21A consensus sequence.

FIG. 148 depicts exemplary sequence analyses performed on amino acid sequences encoded by UL22A of the CMV genome. FIG. 148A shows an identification of exemplary predicted epitopes with respect to positions along a UL22A consensus sequence; FIG. 148B depicts conservation scores determined for amino acids located at positions along a UL22A consensus sequence.

FIG. 149 depicts exemplary sequence analyses performed on amino acid sequences encoded by UL23 of the CMV genome. FIG. 149A shows an identification of exemplary predicted epitopes with respect to positions along a UL23 consensus sequence; FIG. 149B depicts conservation scores determined for amino acids located at positions along a UL23 consensus sequence.

FIG. 150 depicts exemplary sequence analyses performed on amino acid sequences encoded by UL24 of the CMV genome. FIG. 150A shows an identification of exemplary predicted epitopes with respect to positions along a UL24 consensus sequence; FIG. 150B depicts conservation scores determined for amino acids located at positions along a UL24 consensus sequence.

FIG. 151 depicts exemplary sequence analyses performed on amino acid sequences encoded by UL25 of the CMV genome. FIG. 151A shows an identification of exemplary predicted epitopes with respect to positions along a UL25 consensus sequence; FIG. 151B depicts conservation scores determined for amino acids located at positions along a UL25 consensus sequence.

FIG. 152 depicts exemplary sequence analyses performed on amino acid sequences encoded by UL26 of the CMV genome. FIG. 152A shows an identification of exemplary predicted epitopes with respect to positions along a UL26 consensus sequence; FIG. 152B depicts conservation scores determined for amino acids located at positions along a UL26 consensus sequence.

FIG. 153 depicts exemplary sequence analyses performed on amino acid sequences encoded by UL27 of the CMV genome. FIG. 153A shows an identification of exemplary predicted epitopes with respect to positions along a UL27 consensus sequence; FIG. 153B depicts conservation scores determined for amino acids located at positions along a UL27 consensus sequence.

FIG. 154 depicts exemplary sequence analyses performed on amino acid sequences encoded by UL29 of the CMV genome. FIG. 154A shows an identification of exemplary predicted epitopes with respect to positions along a UL29 consensus sequence; FIG. 154B depicts conservation scores determined for amino acids located at positions along a UL29 consensus sequence.

FIG. 155 depicts exemplary sequence analyses performed on amino acid sequences encoded by UL2 of the CMV genome. FIG. 155A shows an identification of exemplary predicted epitopes with respect to positions along a UL2 consensus sequence; FIG. 155B depicts conservation scores determined for amino acids located at positions along a UL2 consensus sequence.

FIG. 156 depicts exemplary sequence analyses performed on amino acid sequences encoded by UL30A of the CMV genome. FIG. 156A shows an identification of exemplary predicted epitopes with respect to positions along a UL30A consensus sequence; FIG. 156B depicts conservation scores determined for amino acids located at positions along a UL30A consensus sequence.

FIG. 157 depicts exemplary sequence analyses performed on amino acid sequences encoded by UL30 of the CMV genome. FIG. 157A shows an identification of exemplary predicted epitopes with respect to positions along a UL30 consensus sequence; FIG. 157B depicts conservation scores determined for amino acids located at positions along a UL30 consensus sequence.

FIG. 158 depicts exemplary sequence analyses performed on amino acid sequences encoded by UL31 of the CMV genome. FIG. 158A shows an identification of exemplary predicted epitopes with respect to positions along a UL31 consensus sequence; FIG. 158B depicts conservation scores determined for amino acids located at positions along a UL31 consensus sequence.

FIG. 159 depicts exemplary sequence analyses performed on amino acid sequences encoded by UL32 of the CMV genome. FIG. 159A shows an identification of exemplary predicted epitopes with respect to positions along a UL32 consensus sequence; FIG. 159B depicts conservation scores determined for amino acids located at positions along a UL32 consensus sequence.

FIG. 160 depicts exemplary sequence analyses performed on amino acid sequences encoded by UL33 of the CMV genome. FIG. 160A shows an identification of exemplary predicted epitopes with respect to positions along a UL33 consensus sequence; FIG. 160B depicts conservation scores determined for amino acids located at positions along a UL33 consensus sequence.

FIG. 161 depicts exemplary sequence analyses performed on amino acid sequences encoded by UL34 of the CMV genome. FIG. 161A shows an identification of exemplary predicted epitopes with respect to positions along a UL34 consensus sequence; FIG. 161B depicts conservation scores determined for amino acids located at positions along a UL34 consensus sequence.

FIG. 162 depicts exemplary sequence analyses performed on amino acid sequences encoded by UL35 of the CMV genome. FIG. 162A shows an identification of exemplary predicted epitopes with respect to positions along a UL35 consensus sequence; FIG. 162B depicts conservation scores determined for amino acids located at positions along a UL35 consensus sequence.

FIG. 163 depicts exemplary sequence analyses performed on amino acid sequences encoded by UL36 of the CMV genome. FIG. 163A shows an identification of exemplary predicted epitopes with respect to positions along a UL36 consensus sequence; FIG. 163B depicts conservation scores determined for amino acids located at positions along a UL36 consensus sequence.

FIG. 164 depicts exemplary sequence analyses performed on amino acid sequences encoded by UL37 of the CMV genome. FIG. 164A shows an identification of exemplary predicted epitopes with respect to positions along a UL37 consensus sequence; FIG. 164B depicts conservation scores determined for amino acids located at positions along a UL37 consensus sequence.

FIG. 165 depicts exemplary sequence analyses performed on amino acid sequences encoded by UL38 of the CMV genome. FIG. 165A shows an identification of exemplary predicted epitopes with respect to positions along a UL38 consensus sequence; FIG. 165B depicts conservation scores determined for amino acids located at positions along a UL38 consensus sequence.

FIG. 166 depicts exemplary sequence analyses performed on amino acid sequences encoded by UL40 of the CMV genome. FIG. 166A shows an identification of exemplary predicted epitopes with respect to positions along a UL40 consensus sequence; FIG. 166B depicts conservation scores determined for amino acids located at positions along a UL40 consensus sequence.

FIG. 167 depicts exemplary sequence analyses performed on amino acid sequences encoded by UL41A of the CMV genome. FIG. 167A shows an identification of exemplary predicted epitopes with respect to positions along a UL41A consensus sequence; FIG. 167B depicts conservation scores determined for amino acids located at positions along a UL41A consensus sequence.

FIG. 168 depicts exemplary sequence analyses performed on amino acid sequences encoded by UL42 of the CMV genome. FIG. 168A shows an identification of exemplary predicted epitopes with respect to positions along a UL42 consensus sequence; FIG. 168B depicts conservation scores determined for amino acids located at positions along a UL42 consensus sequence.

FIG. 169 depicts exemplary sequence analyses performed on amino acid sequences encoded by UL43 of the CMV genome. FIG. 169A shows an identification of exemplary predicted epitopes with respect to positions along a UL43 consensus sequence; FIG. 169B depicts conservation scores determined for amino acids located at positions along a UL43 consensus sequence.

FIG. 170 depicts exemplary sequence analyses performed on amino acid sequences encoded by UL44 of the CMV genome. FIG. 170A shows an identification of exemplary predicted epitopes with respect to positions along a UL44 consensus sequence; FIG. 170B depicts conservation scores determined for amino acids located at positions along a UL44 consensus sequence.

FIG. 171 depicts exemplary sequence analyses performed on amino acid sequences encoded by UL45 of the CMV genome. FIG. 171A shows an identification of exemplary predicted epitopes with respect to positions along a UL45 consensus sequence; FIG. 171B depicts conservation scores determined for amino acids located at positions along a UL45 consensus sequence.

FIG. 172 depicts exemplary sequence analyses performed on amino acid sequences encoded by UL46 of the CMV genome. FIG. 172A shows an identification of exemplary predicted epitopes with respect to positions along a UL46 consensus sequence; FIG. 172B depicts conservation scores determined for amino acids located at positions along a UL46 consensus sequence.

FIG. 173 depicts exemplary sequence analyses performed on amino acid sequences encoded by UL47 of the CMV genome. FIG. 173A shows an identification of exemplary predicted epitopes with respect to positions along a UL47 consensus sequence; FIG. 173B depicts conservation scores determined for amino acids located at positions along a UL47 consensus sequence.

FIG. 174 depicts exemplary sequence analyses performed on amino acid sequences encoded by UL48A of the CMV genome. FIG. 174A shows an identification of exemplary predicted epitopes with respect to positions along a UL48A consensus sequence; FIG. 174B depicts conservation scores determined for amino acids located at positions along a UL48A consensus sequence.

FIG. 175 depicts exemplary sequence analyses performed on amino acid sequences encoded by UL48 of the CMV genome. FIG. 175A shows an identification of exemplary predicted epitopes with respect to positions along a UL48 consensus sequence; FIG. 175B depicts conservation scores determined for amino acids located at positions along a UL48 consensus sequence.

FIG. 176 depicts exemplary sequence analyses performed on amino acid sequences encoded by UL49 of the CMV genome. FIG. 176A shows an identification of exemplary predicted epitopes with respect to positions along a UL49 consensus sequence; FIG. 176B depicts conservation scores determined for amino acids located at positions along a UL49 consensus sequence.

FIG. 177 depicts exemplary sequence analyses performed on amino acid sequences encoded by UL4 of the CMV genome. FIG. 177A shows an identification of exemplary predicted epitopes with respect to positions along a UL4 consensus sequence; FIG. 177B depicts conservation scores determined for amino acids located at positions along a UL4 consensus sequence.

FIG. 178 depicts exemplary sequence analyses performed on amino acid sequences encoded by UL50 of the CMV genome. FIG. 178A shows an identification of exemplary predicted epitopes with respect to positions along a UL50 consensus sequence; FIG. 178B depicts conservation scores determined for amino acids located at positions along a UL50 consensus sequence.

FIG. 179 depicts exemplary sequence analyses performed on amino acid sequences encoded by UL51 of the CMV genome. FIG. 179A shows an identification of exemplary predicted epitopes with respect to positions along a UL51 consensus sequence; FIG. 179B depicts conservation scores determined for amino acids located at positions along a UL51 consensus sequence.

FIG. 180 depicts exemplary sequence analyses performed on amino acid sequences encoded by UL52 of the CMV genome. FIG. 180A shows an identification of exemplary predicted epitopes with respect to positions along a UL52 consensus sequence; FIG. 180B depicts conservation scores determined for amino acids located at positions along a UL52 consensus sequence.

FIG. 181 depicts exemplary sequence analyses performed on amino acid sequences encoded by UL53 of the CMV genome. FIG. 181A shows an identification of exemplary predicted epitopes with respect to positions along a UL53 consensus sequence; FIG. 181B depicts conservation scores determined for amino acids located at positions along a UL53 consensus sequence.

FIG. 182 depicts exemplary sequence analyses performed on amino acid sequences encoded by UL54 of the CMV genome. FIG. 182A shows an identification of exemplary predicted epitopes with respect to positions along a UL54 consensus sequence; FIG. 182B depicts conservation scores determined for amino acids located at positions along a UL54 consensus sequence.

FIG. 183 depicts exemplary sequence analyses performed on amino acid sequences encoded by UL55 of the CMV genome. FIG. 183A shows an identification of exemplary predicted epitopes with respect to positions along a UL55 consensus sequence; FIG. 183B depicts conservation scores determined for amino acids located at positions along a UL55 consensus sequence.

FIG. 184 depicts exemplary sequence analyses performed on amino acid sequences encoded by UL56 of the CMV genome. FIG. 184A shows an identification of exemplary predicted epitopes with respect to positions along a UL56 consensus sequence; FIG. 184B depicts conservation scores determined for amino acids located at positions along a UL56 consensus sequence.

FIG. 185 depicts exemplary sequence analyses performed on amino acid sequences encoded by UL57 of the CMV genome. FIG. 185A shows an identification of exemplary predicted epitopes with respect to positions along a UL57 consensus sequence; FIG. 185B depicts conservation scores determined for amino acids located at positions along a UL57 consensus sequence.

FIG. 186 depicts exemplary sequence analyses performed on amino acid sequences encoded by UL5 of the CMV genome. FIG. 186A shows an identification of exemplary predicted epitopes with respect to positions along a UL5 consensus sequence; FIG. 186B depicts conservation scores determined for amino acids located at positions along a UL5 consensus sequence.

FIG. 187 depicts exemplary sequence analyses performed on amino acid sequences encoded by UL69 of the CMV genome. FIG. 187A shows an identification of exemplary predicted epitopes with respect to positions along a UL69 consensus sequence; FIG. 187B depicts conservation scores determined for amino acids located at positions along a UL69 consensus sequence.

FIG. 188 depicts exemplary sequence analyses performed on amino acid sequences encoded by UL6 of the CMV genome. FIG. 188A shows an identification of exemplary predicted epitopes with respect to positions along a UL6 consensus sequence; FIG. 188B depicts conservation scores determined for amino acids located at positions along a UL6 consensus sequence.

FIG. 189 depicts exemplary sequence analyses performed on amino acid sequences encoded by UL70 of the CMV genome. FIG. 189A shows an identification of exemplary predicted epitopes with respect to positions along a UL70 consensus sequence; FIG. 189B depicts conservation scores determined for amino acids located at positions along a UL70 consensus sequence.

FIG. 190 depicts exemplary sequence analyses performed on amino acid sequences encoded by UL71 of the CMV genome. FIG. 190A shows an identification of exemplary predicted epitopes with respect to positions along a UL71 consensus sequence; FIG. 190B depicts conservation scores determined for amino acids located at positions along a UL71 consensus sequence.

FIG. 191 depicts exemplary sequence analyses performed on amino acid sequences encoded by UL72 of the CMV genome. FIG. 191A shows an identification of exemplary predicted epitopes with respect to positions along a UL72 consensus sequence; FIG. 191B depicts conservation scores determined for amino acids located at positions along a UL72 consensus sequence.

FIG. 192 depicts exemplary sequence analyses performed on amino acid sequences encoded by UL73 of the CMV genome. FIG. 192A shows an identification of exemplary predicted epitopes with respect to positions along a UL73 consensus sequence; FIG. 192B depicts conservation scores determined for amino acids located at positions along a UL73 consensus sequence.

FIG. 193 depicts exemplary sequence analyses performed on amino acid sequences encoded by UL74A of the CMV genome. FIG. 193A shows an identification of exemplary predicted epitopes with respect to positions along a UL74A consensus sequence; FIG. 193B depicts conservation scores determined for amino acids located at positions along a UL74A consensus sequence.

FIG. 194 depicts exemplary sequence analyses performed on amino acid sequences encoded by UL74 of the CMV genome. FIG. 194A shows an identification of exemplary predicted epitopes with respect to positions along a UL74 consensus sequence; FIG. 194B depicts conservation scores determined for amino acids located at positions along a UL74 consensus sequence.

FIG. 195 depicts exemplary sequence analyses performed on amino acid sequences encoded by UL75 of the CMV genome. FIG. 195A shows an identification of exemplary predicted epitopes with respect to positions along a UL75 consensus sequence; FIG. 195B depicts conservation scores determined for amino acids located at positions along a UL75 consensus sequence.

FIG. 196 depicts exemplary sequence analyses performed on amino acid sequences encoded by UL76 of the CMV genome. FIG. 196A shows an identification of exemplary predicted epitopes with respect to positions along a UL76 consensus sequence; FIG. 196B depicts conservation scores determined for amino acids located at positions along a UL76 consensus sequence.

FIG. 197 depicts exemplary sequence analyses performed on amino acid sequences encoded by UL77 of the CMV genome. FIG. 197A shows an identification of exemplary predicted epitopes with respect to positions along a UL77 consensus sequence; FIG. 197B depicts conservation scores determined for amino acids located at positions along a UL77 consensus sequence.

FIG. 198 depicts exemplary sequence analyses performed on amino acid sequences encoded by UL78 of the CMV genome. FIG. 198A shows an identification of exemplary predicted epitopes with respect to positions along a UL78 consensus sequence; FIG. 198B depicts conservation scores determined for amino acids located at positions along a UL78 consensus sequence.

FIG. 199 depicts exemplary sequence analyses performed on amino acid sequences encoded by UL79 of the CMV genome. FIG. 199A shows an identification of exemplary predicted epitopes with respect to positions along a UL79 consensus sequence; FIG. 199B depicts conservation scores determined for amino acids located at positions along a UL79 consensus sequence.

FIG. 200 depicts exemplary sequence analyses performed on amino acid sequences encoded by UL7 of the CMV genome. FIG. 200A shows an identification of exemplary predicted epitopes with respect to positions along a UL7 consensus sequence; FIG. 200B depicts conservation scores determined for amino acids located at positions along a UL7 consensus sequence.

FIG. 201 depicts exemplary sequence analyses performed on amino acid sequences encoded by UL80 of the CMV genome. FIG. 201A shows an identification of exemplary predicted epitopes with respect to positions along a UL80 consensus sequence; FIG. 201B depicts conservation scores determined for amino acids located at positions along a UL80 consensus sequence.

FIG. 202 depicts exemplary sequence analyses performed on amino acid sequences encoded by UL82 of the CMV genome. FIG. 202A shows an identification of exemplary predicted epitopes with respect to positions along a UL82 consensus sequence; FIG. 202B depicts conservation scores determined for amino acids located at positions along a UL82 consensus sequence.

FIG. 203 depicts exemplary sequence analyses performed on amino acid sequences encoded by UL83 of the CMV genome. FIG. 203A shows an identification of exemplary predicted epitopes with respect to positions along a UL83 consensus sequence; FIG. 203B depicts conservation scores determined for amino acids located at positions along a UL83 consensus sequence.

FIG. 204 depicts exemplary sequence analyses performed on amino acid sequences encoded by UL84 of the CMV genome. FIG. 204A shows an identification of exemplary predicted epitopes with respect to positions along a UL84 consensus sequence; FIG. 204B depicts conservation scores determined for amino acids located at positions along a UL84 consensus sequence.

FIG. 205 depicts exemplary sequence analyses performed on amino acid sequences encoded by UL85 of the CMV genome. FIG. 205A shows an identification of exemplary predicted epitopes with respect to positions along a UL85 consensus sequence; FIG. 205B depicts conservation scores determined for amino acids located at positions along a UL85 consensus sequence.

FIG. 206 depicts exemplary sequence analyses performed on amino acid sequences encoded by UL86 of the CMV genome. FIG. 206A shows an identification of exemplary predicted epitopes with respect to positions along a UL86 consensus sequence; FIG. 206B depicts conservation scores determined for amino acids located at positions along a UL86 consensus sequence.

FIG. 207 depicts exemplary sequence analyses performed on amino acid sequences encoded by UL87 of the CMV genome. FIG. 207A shows an identification of exemplary predicted epitopes with respect to positions along a UL87 consensus sequence; FIG. 207B depicts conservation scores determined for amino acids located at positions along a UL87 consensus sequence.

FIG. 208 depicts exemplary sequence analyses performed on amino acid sequences encoded by UL88 of the CMV genome. FIG. 208A shows an identification of exemplary predicted epitopes with respect to positions along a UL88 consensus sequence; FIG. 208B depicts conservation scores determined for amino acids located at positions along a UL88 consensus sequence.

FIG. 209 depicts exemplary sequence analyses performed on amino acid sequences encoded by UL89 of the CMV genome. FIG. 209A shows an identification of exemplary predicted epitopes with respect to positions along a UL89 consensus sequence; FIG. 209B depicts conservation scores determined for amino acids located at positions along a UL89 consensus sequence.

FIG. 210 depicts exemplary sequence analyses performed on amino acid sequences encoded by UL8 of the CMV genome. FIG. 210A shows an identification of exemplary predicted epitopes with respect to positions along a UL8 consensus sequence; FIG. 210B depicts conservation scores determined for amino acids located at positions along a UL8 consensus sequence.

FIG. 211 depicts exemplary sequence analyses performed on amino acid sequences encoded by UL91 of the CMV genome. FIG. 211A shows an identification of exemplary predicted epitopes with respect to positions along a UL91 consensus sequence; FIG. 211B depicts conservation scores determined for amino acids located at positions along a UL91 consensus sequence.

FIG. 212 depicts exemplary sequence analyses performed on amino acid sequences encoded by UL92 of the CMV genome. FIG. 212A shows an identification of exemplary predicted epitopes with respect to positions along a UL92 consensus sequence; FIG. 212B depicts conservation scores determined for amino acids located at positions along a UL92 consensus sequence.

FIG. 213 depicts exemplary sequence analyses performed on amino acid sequences encoded by UL93 of the CMV genome. FIG. 213A shows an identification of exemplary predicted epitopes with respect to positions along a UL93 consensus sequence; FIG. 213B depicts conservation scores determined for amino acids located at positions along a UL93 consensus sequence.

FIG. 214 depicts exemplary sequence analyses performed on amino acid sequences encoded by UL94 of the CMV genome. FIG. 214A shows an identification of exemplary predicted epitopes with respect to positions along a UL94 consensus sequence; FIG. 214B depicts conservation scores determined for amino acids located at positions along a UL94 consensus sequence.

FIG. 215 depicts exemplary sequence analyses performed on amino acid sequences encoded by UL95 of the CMV genome. FIG. 215A shows an identification of exemplary predicted epitopes with respect to positions along a UL95 consensus sequence; FIG. 215B depicts conservation scores determined for amino acids located at positions along a UL95 consensus sequence.

FIG. 216 depicts exemplary sequence analyses performed on amino acid sequences encoded by UL96 of the CMV genome. FIG. 216A shows an identification of exemplary predicted epitopes with respect to positions along a UL96 consensus sequence; FIG. 216B depicts conservation scores determined for amino acids located at positions along a UL96 consensus sequence.

FIG. 217 depicts exemplary sequence analyses performed on amino acid sequences encoded by UL97 of the CMV genome. FIG. 217A shows an identification of exemplary predicted epitopes with respect to positions along a UL97 consensus sequence; FIG. 217B depicts conservation scores determined for amino acids located at positions along a UL97 consensus sequence.

FIG. 218 depicts exemplary sequence analyses performed on amino acid sequences encoded by UL98 of the CMV genome. FIG. 218A shows an identification of exemplary predicted epitopes with respect to positions along a UL98 consensus sequence; FIG. 218B depicts conservation scores determined for amino acids located at positions along a UL98 consensus sequence.

FIG. 219 depicts exemplary sequence analyses performed on amino acid sequences encoded by UL99 of the CMV genome. FIG. 219A shows an identification of exemplary predicted epitopes with respect to positions along a UL99 consensus sequence; FIG. 219B depicts conservation scores determined for amino acids located at positions along a UL99 consensus sequence.

FIG. 220 depicts exemplary sequence analyses performed on amino acid sequences encoded by UL9 of the CMV genome. FIG. 220A shows an identification of exemplary predicted epitopes with respect to positions along a UL9 consensus sequence; FIG. 220B depicts conservation scores determined for amino acids located at positions along a UL9 consensus sequence.

FIG. 221 depicts exemplary sequence analyses performed on amino acid sequences encoded by US10 of the CMV genome. FIG. 221A shows an identification of exemplary predicted epitopes with respect to positions along a US10 consensus sequence; FIG. 221B depicts conservation scores determined for amino acids located at positions along a US10 consensus sequence.

FIG. 222 depicts exemplary sequence analyses performed on amino acid sequences encoded by US11 of the CMV genome. FIG. 222A shows an identification of exemplary predicted epitopes with respect to positions along a US11 consensus sequence; FIG. 222B depicts conservation scores determined for amino acids located at positions along a US11 consensus sequence.

FIG. 223 depicts exemplary sequence analyses performed on amino acid sequences encoded by US12 of the CMV genome. FIG. 223A shows an identification of exemplary predicted epitopes with respect to positions along a US12 consensus sequence; FIG. 223B depicts conservation scores determined for amino acids located at positions along a US12 consensus sequence.

FIG. 224 depicts exemplary sequence analyses performed on amino acid sequences encoded by US13 of the CMV genome. FIG. 224A shows an identification of exemplary predicted epitopes with respect to positions along a US13 consensus sequence; FIG. 224B depicts conservation scores determined for amino acids located at positions along a US13 consensus sequence.

FIG. 225 depicts exemplary sequence analyses performed on amino acid sequences encoded by US14 of the CMV genome. FIG. 225A shows an identification of exemplary predicted epitopes with respect to positions along a US14 consensus sequence; FIG. 225B depicts conservation scores determined for amino acids located at positions along a US14 consensus sequence.

FIG. 226 depicts exemplary sequence analyses performed on amino acid sequences encoded by US15 of the CMV genome. FIG. 226A shows an identification of exemplary predicted epitopes with respect to positions along a US15 consensus sequence; FIG. 226B depicts conservation scores determined for amino acids located at positions along a US15 consensus sequence.

FIG. 227 depicts exemplary sequence analyses performed on amino acid sequences encoded by US16 of the CMV genome. FIG. 227A shows an identification of exemplary predicted epitopes with respect to positions along a US16 consensus sequence; FIG. 227B depicts conservation scores determined for amino acids located at positions along a US16 consensus sequence.

FIG. 228 depicts exemplary sequence analyses performed on amino acid sequences encoded by US17 of the CMV genome. FIG. 228A shows an identification of exemplary predicted epitopes with respect to positions along a US17 consensus sequence; FIG. 228B depicts conservation scores determined for amino acids located at positions along a US17 consensus sequence.

FIG. 229 depicts exemplary sequence analyses performed on amino acid sequences encoded by US18 of the CMV genome. FIG. 229A shows an identification of exemplary predicted epitopes with respect to positions along a US18 consensus sequence; FIG. 229B depicts conservation scores determined for amino acids located at positions along a US18 consensus sequence.

FIG. 230 depicts exemplary sequence analyses performed on amino acid sequences encoded by US19 of the CMV genome. FIG. 230A shows an identification of exemplary predicted epitopes with respect to positions along a US19 consensus sequence; FIG. 230B depicts conservation scores determined for amino acids located at positions along a US19 consensus sequence.

FIG. 231 depicts exemplary sequence analyses performed on amino acid sequences encoded by US1 of the CMV genome. FIG. 231A shows an identification of exemplary predicted epitopes with respect to positions along a US1 consensus sequence; FIG. 231B depicts conservation scores determined for amino acids located at positions along a US1 consensus sequence.

FIG. 232 depicts exemplary sequence analyses performed on amino acid sequences encoded by US20 of the CMV genome. FIG. 232A shows an identification of exemplary predicted epitopes with respect to positions along a US20 consensus sequence; FIG. 232B depicts conservation scores determined for amino acids located at positions along a US20 consensus sequence.

FIG. 233 depicts exemplary sequence analyses performed on amino acid sequences encoded by US21 of the CMV genome. FIG. 233A shows an identification of exemplary predicted epitopes with respect to positions along a US21 consensus sequence; FIG. 233B depicts conservation scores determined for amino acids located at positions along a US21 consensus sequence.

FIG. 234 depicts exemplary sequence analyses performed on amino acid sequences encoded by US22 of the CMV genome. FIG. 234A shows an identification of exemplary predicted epitopes with respect to positions along a US22 consensus sequence; FIG. 234B depicts conservation scores determined for amino acids located at positions along a US22 consensus sequence.

FIG. 235 depicts exemplary sequence analyses performed on amino acid sequences encoded by US23 of the CMV genome. FIG. 235A shows an identification of exemplary predicted epitopes with respect to positions along a US23 consensus sequence; FIG. 235B depicts conservation scores determined for amino acids located at positions along a US23 consensus sequence.

FIG. 236 depicts exemplary sequence analyses performed on amino acid sequences encoded by US24 of the CMV genome. FIG. 236A shows an identification of exemplary predicted epitopes with respect to positions along a US24 consensus sequence; FIG. 236B depicts conservation scores determined for amino acids located at positions along a US24 consensus sequence.

FIG. 237 depicts exemplary sequence analyses performed on amino acid sequences encoded by US26 of the CMV genome. FIG. 237A shows an identification of exemplary predicted epitopes with respect to positions along a US26 consensus sequence; FIG. 237B depicts conservation scores determined for amino acids located at positions along a US26 consensus sequence.

FIG. 238 depicts exemplary sequence analyses performed on amino acid sequences encoded by US27 of the CMV genome. FIG. 238A shows an identification of exemplary predicted epitopes with respect to positions along a US27 consensus sequence; FIG. 238B depicts conservation scores determined for amino acids located at positions along a US27 consensus sequence.

FIG. 239 depicts exemplary sequence analyses performed on amino acid sequences encoded by US28 of the CMV genome. FIG. 239A shows an identification of exemplary predicted epitopes with respect to positions along a US28 consensus sequence; FIG. 239B depicts conservation scores determined for amino acids located at positions along a US28 consensus sequence.

FIG. 240 depicts exemplary sequence analyses performed on amino acid sequences encoded by US29 of the CMV genome. FIG. 240A shows an identification of exemplary predicted epitopes with respect to positions along a US29 consensus sequence; FIG. 240B depicts conservation scores determined for amino acids located at positions along a US29 consensus sequence.

FIG. 241 depicts exemplary sequence analyses performed on amino acid sequences encoded by US30 of the CMV genome. FIG. 241A shows an identification of exemplary predicted epitopes with respect to positions along a US30 consensus sequence; FIG. 241B depicts conservation scores determined for amino acids located at positions along a US30 consensus sequence.

FIG. 242 depicts exemplary sequence analyses performed on amino acid sequences encoded by US31 of the CMV genome. FIG. 242A shows an identification of exemplary predicted epitopes with respect to positions along a US31 consensus sequence; FIG. 242B depicts conservation scores determined for amino acids located at positions along a US31 consensus sequence.

FIG. 243 depicts exemplary sequence analyses performed on amino acid sequences encoded by US32 of the CMV genome. FIG. 243A shows an identification of exemplary predicted epitopes with respect to positions along a US32 consensus sequence; FIG. 243B depicts conservation scores determined for amino acids located at positions along a US32 consensus sequence.

FIG. 244 depicts exemplary sequence analyses performed on amino acid sequences encoded by US33A of the CMV genome. FIG. 244A shows an identification of exemplary predicted epitopes with respect to positions along a US33A consensus sequence; FIG. 244B depicts conservation scores determined for amino acids located at positions along a US33A consensus sequence.

FIG. 245 depicts exemplary sequence analyses performed on amino acid sequences encoded by US34A of the CMV genome. FIG. 245A shows an identification of exemplary predicted epitopes with respect to positions along a US34A consensus sequence; FIG. 245B depicts conservation scores determined for amino acids located at positions along a US34A consensus sequence.

FIG. 246 depicts exemplary sequence analyses performed on amino acid sequences encoded by US34 of the CMV genome. FIG. 246A shows an identification of exemplary predicted epitopes with respect to positions along a US34 consensus sequence; FIG. 246B depicts conservation scores determined for amino acids located at positions along a US34 consensus sequence.

FIG. 247 depicts exemplary sequence analyses performed on amino acid sequences encoded by US3 of the CMV genome. FIG. 247A shows an identification of exemplary predicted epitopes with respect to positions along a US3 consensus sequence; FIG. 247B depicts conservation scores determined for amino acids located at positions along a US3 consensus sequence.

FIG. 248 depicts exemplary sequence analyses performed on amino acid sequences encoded by US6 of the CMV genome. FIG. 248A shows an identification of exemplary predicted epitopes with respect to positions along a US6 consensus sequence; FIG. 248B depicts conservation scores determined for amino acids located at positions along a US6 consensus sequence.

FIG. 249 depicts exemplary sequence analyses performed on amino acid sequences encoded by US7 of the CMV genome. FIG. 249A shows an identification of exemplary predicted epitopes with respect to positions along a US7 consensus sequence; FIG. 249B depicts conservation scores determined for amino acids located at positions along a US7 consensus sequence.

FIG. 250 depicts exemplary sequence analyses performed on amino acid sequences encoded by US8 of the CMV genome. FIG. 250A shows an identification of exemplary predicted epitopes with respect to positions along a US8 consensus sequence; FIG. 250B depicts conservation scores determined for amino acids located at positions along a US8 consensus sequence.

FIG. 251 depicts exemplary sequence analyses performed on amino acid sequences encoded by US9 of the CMV genome. FIG. 251A shows an identification of exemplary predicted epitopes with respect to positions along a US9 consensus sequence; FIG. 251B depicts conservation scores determined for amino acids located at positions along a US9 consensus sequence.

FIG. 252 has been modified from Sandonis, et al., “Role of Neutralizing Antibodies in CMV Infection: Implications for New Therapeutic Approaches,” Trends in Microbiology, 28:11 (November 2020), which is incorporated herein by reference in its entirety. FIG. 252 includes a schematic representation of the CMV virion. As illustrated, an outer membrane of CMV has multiple embedded glycoprotein complexes. The gCI complex includes gB, the gCII complex includes gM and gN, the gCIII complex includes gH, gL, and gO, and the pentameric complex includes gH/gL heterodimer bound to three small glycoproteins encoded by UL128, UL130, and UL131. The gCII (gM/gN) is involved in the initial attachment with the cell though interaction with glycosaminoglycans. For fibroblasts and Langerhans cells, viral entry is mediated by gB and gH/gL/gO, while entry into epithelial, endothelial, and myeloid cells occurs through the interaction between the pentameric complex: gH/gL/pUL128-pUL130-pUL131A (indicated as gH/gL/UL128-131) and the cell receptor.

FIG. 253 has been modified from Jean Beltran, P. M., et al., “The life cycle and pathogenesis of human cytomegalovirus infection: lessons from proteomics,” Expert Rev Proteomics, 11(6): 697-711 (December 2014), which is incorporated herein by reference in its entirety. FIG. 253 depicts a schematic overview of the CMV life cycle.

FIG. 254 has been modified from Jean Beltran (2014), which is incorporated herein by reference in its entirety. FIG. 254 depicts a schematic of examples of virus-host interactions during the CMV replication cycle. Panel (A) depicts interactions resulting in host and virus control of immediate early (IE) gene expression, Panel (B) depicts protein complexes involved in HCMV genome replication, Panel (C) depicts proteins involved in viral modulation of cellular stress response through TSC1/2, and Panel (D) depicts proteins and complexes involved in control of cell cycle progression and induction of the DNA damage response.

FIG. 255 presents an optional immunization protocol for mouse studies.

FIG. 256 presents and exemplary workflow for identification, selection and/or characterization of antigens (e.g., CMV proteins, including particular variants, and/or epitopes thereof, in particular T cell epitopes) for use in accordance with the present disclosure.

FIG. 257 is a visual representation of norovirus strains grouped by their sequence similarity. Each cell in the square gird represents the sequence similarity of a pair of strains according to a sliding color scale in which blue represents poor homology, white indicates moderate homology, and red indicates strong homology. The ordering of strains is identical for both rows and columns and is permuted such that clusters of similar sequences are evident. Dendrograms likewise display the presence of related strain groupings.

FIG. 258 depicts exemplary sequence analyses performed on amino acid sequences encoded by NTPase of the Norovirus GI genome. FIG. 258A shows an identification of exemplary predicted epitopes with respect to positions along an NTPase consensus sequence; FIG. 258B depicts conservation scores determined for amino acids located at positions along an NTPase consensus sequence.

FIG. 259 depicts exemplary sequence analyses performed on amino acid sequences encoded by Nterm of the Norovirus GI genome. FIG. 259A shows an identification of exemplary predicted epitopes with respect to positions along an Nterm consensus sequence; FIG. 259B depicts conservation scores determined for amino acids located at positions along an Nterm consensus sequence.

FIG. 260 depicts exemplary sequence analyses performed on amino acid sequences encoded by Pro of the Norovirus GI genome. FIG. 260A shows an identification of exemplary predicted epitopes with respect to positions along a Pro consensus sequence; FIG. 260B depicts conservation scores determined for amino acids located at positions along a Pro consensus sequence.

FIG. 261 depicts exemplary sequence analyses performed on amino acid sequences encoded by RdRp of the Norovirus GI genome. FIG. 261A shows an identification of exemplary predicted epitopes with respect to positions along an RdRp consensus sequence; FIG. 261B depicts conservation scores determined for amino acids located at positions along an RdRp consensus sequence.

FIG. 262 depicts exemplary sequence analyses performed on amino acid sequences encoded by VP1 of the Norovirus GI genome. FIG. 262A shows an identification of exemplary predicted epitopes with respect to positions along a VP1 consensus sequence; FIG. 262B depicts conservation scores determined for amino acids located at positions along a VP1 consensus sequence.

FIG. 263 depicts exemplary sequence analyses performed on amino acid sequences encoded by VP2 of the Norovirus GI genome. FIG. 263A shows an identification of exemplary predicted epitopes with respect to positions along a VP2 consensus sequence; FIG. 263B depicts conservation scores determined for amino acids located at positions along a VP2 consensus sequence.

FIG. 264 depicts exemplary sequence analyses performed on amino acid sequences encoded by VPg of the Norovirus GI genome. FIG. 264A shows an identification of exemplary predicted epitopes with respect to positions along a VPg consensus sequence; FIG. 264B depicts conservation scores determined for amino acids located at positions along a VPg consensus sequence.

FIG. 265 depicts exemplary sequence analyses performed on amino acid sequences encoded by p22 of the Norovirus GI genome. FIG. 265A shows an identification of exemplary predicted epitopes with respect to positions along a p22 consensus sequence; FIG. 265B depicts conservation scores determined for amino acids located at positions along a p22 consensus sequence.

FIG. 266 depicts exemplary sequence analyses performed on amino acid sequences encoded by NTPase of the Norovirus GII.P4 genome. FIG. 266A shows an identification of exemplary predicted epitopes with respect to positions along an NTPase consensus sequence; FIG. 266B depicts conservation scores determined for amino acids located at positions along an NTPase consensus sequence.

FIG. 267 depicts exemplary sequence analyses performed on amino acid sequences encoded by Nterm of the Norovirus GII.P4 genome. FIG. 267A shows an identification of exemplary predicted epitopes with respect to positions along an Nterm consensus sequence; FIG. 267B depicts conservation scores determined for amino acids located at positions along an Nterm consensus sequence.

FIG. 268 depicts exemplary sequence analyses performed on amino acid sequences encoded by Pro of the Norovirus GII.P4 genome. FIG. 268A shows an identification of exemplary predicted epitopes with respect to positions along a Pro consensus sequence; FIG. 268B depicts conservation scores determined for amino acids located at positions along a Pro consensus sequence.

FIG. 269 depicts exemplary sequence analyses performed on amino acid sequences encoded by RdRp of the Norovirus GII.P4 genome. FIG. 269A shows an identification of exemplary predicted epitopes with respect to positions along an RdRp consensus sequence; FIG. 269B depicts conservation scores determined for amino acids located at positions along an RdRp consensus sequence.

FIG. 270 depicts exemplary sequence analyses performed on amino acid sequences encoded by VP1 of the Norovirus GII.P4 genome. FIG. 270A shows an identification of exemplary predicted epitopes with respect to positions along a VP1 consensus sequence; FIG. 270B depicts conservation scores determined for amino acids located at positions along a VP1 consensus sequence.

FIG. 271 depicts exemplary sequence analyses performed on amino acid sequences encoded by VP2 of the Norovirus GII.P4 genome. FIG. 271A shows an identification of exemplary predicted epitopes with respect to positions along a VP2 consensus sequence; FIG. 271B depicts conservation scores determined for amino acids located at positions along a VP2 consensus sequence.

FIG. 272 depicts exemplary sequence analyses performed on amino acid sequences encoded by VPg of the Norovirus GII.P4 genome. FIG. 272A shows an identification of exemplary predicted epitopes with respect to positions along a VPg consensus sequence; FIG. 272B depicts conservation scores determined for amino acids located at positions along a VPg consensus sequence.

FIG. 273 depicts exemplary sequence analyses performed on amino acid sequences encoded by p22 of the Norovirus GII.P4 genome. FIG. 273A shows an identification of exemplary predicted epitopes with respect to positions along a p22 consensus sequence; FIG. 273B depicts conservation scores determined for amino acids located at positions along a p22 consensus sequence.

FIG. 274 depicts exemplary sequence analyses performed on amino acid sequences encoded by NTPase of the Norovirus GII.P7 genome. FIG. 274A shows an identification of exemplary predicted epitopes with respect to positions along an NTPase consensus sequence; FIG. 274B depicts conservation scores determined for amino acids located at positions along an NTPase consensus sequence.

FIG. 275 depicts exemplary sequence analyses performed on amino acid sequences encoded by Nterm of the Norovirus GII.P7 genome. FIG. 275A shows an identification of exemplary predicted epitopes with respect to positions along an Nterm consensus sequence; FIG. 275B depicts conservation scores determined for amino acids located at positions along an Nterm consensus sequence.

FIG. 276 depicts exemplary sequence analyses performed on amino acid sequences encoded by Pro of the Norovirus GII.P7 genome. FIG. 276A shows an identification of exemplary predicted epitopes with respect to positions along a Pro consensus sequence; FIG. 276B depicts conservation scores determined for amino acids located at positions along a Pro consensus sequence.

FIG. 277 depicts exemplary sequence analyses performed on amino acid sequences encoded by RdRp of the Norovirus GII.P7 genome. FIG. 277A shows an identification of exemplary predicted epitopes with respect to positions along an RdRp consensus sequence; FIG. 277B depicts conservation scores determined for amino acids located at positions along an RdRp consensus sequence.

FIG. 278 depicts exemplary sequence analyses performed on amino acid sequences encoded by VP1 of the Norovirus GII.P7 genome. FIG. 278A shows an identification of exemplary predicted epitopes with respect to positions along a VP1 consensus sequence; FIG. 278B depicts conservation scores determined for amino acids located at positions along a VP1 consensus sequence.

FIG. 279 depicts exemplary sequence analyses performed on amino acid sequences encoded by VP2 of the Norovirus GII.P7 genome. FIG. 279A shows an identification of exemplary predicted epitopes with respect to positions along a VP2 consensus sequence; FIG. 279B depicts conservation scores determined for amino acids located at positions along a VP2 consensus sequence.

FIG. 280 depicts exemplary sequence analyses performed on amino acid sequences encoded by VPg of the Norovirus GII.P7 genome. FIG. 280A shows an identification of exemplary predicted epitopes with respect to positions along a VPg consensus sequence; FIG. 280B depicts conservation scores determined for amino acids located at positions along a VPg consensus sequence.

FIG. 281 depicts exemplary sequence analyses performed on amino acid sequences encoded by p22 of the Norovirus GII.P7 genome. FIG. 281A shows an identification of exemplary predicted epitopes with respect to positions along a p22 consensus sequence; FIG. 281B depicts conservation scores determined for amino acids located at positions along a p22 consensus sequence.

FIG. 282 depicts exemplary sequence analyses performed on amino acid sequences encoded by NTPase of the Norovirus GII.P12 genome. FIG. 282A shows an identification of exemplary predicted epitopes with respect to positions along an NTPase consensus sequence; FIG. 282B depicts conservation scores determined for amino acids located at positions along an NTPase consensus sequence.

FIG. 283 depicts exemplary sequence analyses performed on amino acid sequences encoded by Nterm of the Norovirus GII.P12 genome. FIG. 283A shows an identification of exemplary predicted epitopes with respect to positions along an Nterm consensus sequence; FIG. 283B depicts conservation scores determined for amino acids located at positions along an Nterm consensus sequence.

FIG. 284 depicts exemplary sequence analyses performed on amino acid sequences encoded by Pro of the Norovirus GII.P12 genome. FIG. 284A shows an identification of exemplary predicted epitopes with respect to positions along a Pro consensus sequence; FIG. 284B depicts conservation scores determined for amino acids located at positions along a Pro consensus sequence.

FIG. 285 depicts exemplary sequence analyses performed on amino acid sequences encoded by RdRp of the Norovirus GII.P12 genome. FIG. 285A shows an identification of exemplary predicted epitopes with respect to positions along an RdRp consensus sequence; FIG. 285B depicts conservation scores determined for amino acids located at positions along an RdRp consensus sequence.

FIG. 286 depicts exemplary sequence analyses performed on amino acid sequences encoded by VP1 of the Norovirus GII.P12 genome. FIG. 286A shows an identification of exemplary predicted epitopes with respect to positions along a VP1 consensus sequence; FIG. 286B depicts conservation scores determined for amino acids located at positions along a VP1 consensus sequence.

FIG. 287 depicts exemplary sequence analyses performed on amino acid sequences encoded by VP2 of the Norovirus GII.P12 genome. FIG. 287A shows an identification of exemplary predicted epitopes with respect to positions along a VP2 consensus sequence; FIG. 287B depicts conservation scores determined for amino acids located at positions along a VP2 consensus sequence.

FIG. 288 depicts exemplary sequence analyses performed on amino acid sequences encoded by VPg of the Norovirus GII.P12 genome. FIG. 288A shows an identification of exemplary predicted epitopes with respect to positions along a VPg consensus sequence; FIG. 288B depicts conservation scores determined for amino acids located at positions along a VPg consensus sequence.

FIG. 289 depicts exemplary sequence analyses performed on amino acid sequences encoded by p22 of the Norovirus GII.P12 genome. FIG. 289A shows an identification of exemplary predicted epitopes with respect to positions along a p22 consensus sequence; FIG. 289B depicts conservation scores determined for amino acids located at positions along a p22 consensus sequence.

FIG. 290 depicts exemplary sequence analyses performed on amino acid sequences encoded by NTPase of the Norovirus GII.P16 genome. FIG. 290A shows an identification of exemplary predicted epitopes with respect to positions along an NTPase consensus sequence; FIG. 290B depicts conservation scores determined for amino acids located at positions along an NTPase consensus sequence.

FIG. 291 depicts exemplary sequence analyses performed on amino acid sequences encoded by Nterm of the Norovirus GII.P16 genome. FIG. 291A shows an identification of exemplary predicted epitopes with respect to positions along an Nterm consensus sequence; FIG. 291B depicts conservation scores determined for amino acids located at positions along an Nterm consensus sequence.

FIG. 292 depicts exemplary sequence analyses performed on amino acid sequences encoded by Pro of the Norovirus GII.P16 genome. FIG. 292A shows an identification of exemplary predicted epitopes with respect to positions along a Pro consensus sequence; FIG. 292B depicts conservation scores determined for amino acids located at positions along a Pro consensus sequence.

FIG. 293 depicts exemplary sequence analyses performed on amino acid sequences encoded by RdRp of the Norovirus GII.P16 genome. FIG. 293A shows an identification of exemplary predicted epitopes with respect to positions along an RdRp consensus sequence; FIG. 293B depicts conservation scores determined for amino acids located at positions along an RdRp consensus sequence.

FIG. 294 depicts exemplary sequence analyses performed on amino acid sequences encoded by VP1 of the Norovirus GII.P16 genome. FIG. 294A shows an identification of exemplary predicted epitopes with respect to positions along a VP1 consensus sequence; FIG. 294B depicts conservation scores determined for amino acids located at positions along a VP1 consensus sequence.

FIG. 295 depicts exemplary sequence analyses performed on amino acid sequences encoded by VP2 of the Norovirus GII.P16 genome. FIG. 295A shows an identification of exemplary predicted epitopes with respect to positions along a VP2 consensus sequence; FIG. 295B depicts conservation scores determined for amino acids located at positions along a VP2 consensus sequence.

FIG. 296 depicts exemplary sequence analyses performed on amino acid sequences encoded by VPg of the Norovirus GII.P16 genome. FIG. 296A shows an identification of exemplary predicted epitopes with respect to positions along a VPg consensus sequence; FIG. 296B depicts conservation scores determined for amino acids located at positions along a VPg consensus sequence.

FIG. 297 depicts exemplary sequence analyses performed on amino acid sequences encoded by p22 of the Norovirus GII.P16 genome. FIG. 297A shows an identification of exemplary predicted epitopes with respect to positions along a p22 consensus sequence; FIG. 297B depicts conservation scores determined for amino acids located at positions along a p22 consensus sequence.

FIG. 298 depicts exemplary sequence analyses performed on amino acid sequences encoded by NTPase of the Norovirus GII.P17 genome. FIG. 298A shows an identification of exemplary predicted epitopes with respect to positions along an NTPase consensus sequence; FIG. 298B depicts conservation scores determined for amino acids located at positions along an NTPase consensus sequence.

FIG. 299 depicts exemplary sequence analyses performed on amino acid sequences encoded by Nterm of the Norovirus GII.P17 genome. FIG. 299A shows an identification of exemplary predicted epitopes with respect to positions along an Nterm consensus sequence; FIG. 299B depicts conservation scores determined for amino acids located at positions along an Nterm consensus sequence.

FIG. 300 depicts exemplary sequence analyses performed on amino acid sequences encoded by Pro of the Norovirus GII.P17 genome. FIG. 300A shows an identification of exemplary predicted epitopes with respect to positions along a Pro consensus sequence; FIG. 300B depicts conservation scores determined for amino acids located at positions along a Pro consensus sequence.

FIG. 301 depicts exemplary sequence analyses performed on amino acid sequences encoded by RdRp of the Norovirus GII.P17 genome. FIG. 301A shows an identification of exemplary predicted epitopes with respect to positions along an RdRp consensus sequence; FIG. 301B depicts conservation scores determined for amino acids located at positions along an RdRp consensus sequence.

FIG. 302 depicts exemplary sequence analyses performed on amino acid sequences encoded by VP1 of the Norovirus GII.P17 genome. FIG. 302A shows an identification of exemplary predicted epitopes with respect to positions along a VP1 consensus sequence; FIG. 302B depicts conservation scores determined for amino acids located at positions along a VP1 consensus sequence.

FIG. 303 depicts exemplary sequence analyses performed on amino acid sequences encoded by VP2 of the Norovirus GII.P17 genome. FIG. 303A shows an identification of exemplary predicted epitopes with respect to positions along a VP2 consensus sequence; FIG. 303B depicts conservation scores determined for amino acids located at positions along a VP2 consensus sequence.

FIG. 304 depicts exemplary sequence analyses performed on amino acid sequences encoded by VPg of the Norovirus GII.P17 genome. FIG. 304A shows an identification of exemplary predicted epitopes with respect to positions along a VPg consensus sequence; FIG. 304B depicts conservation scores determined for amino acids located at positions along a VPg consensus sequence.

FIG. 305 depicts exemplary sequence analyses performed on amino acid sequences encoded by p22 of the Norovirus GII.P17 genome. FIG. 305A shows an identification of exemplary predicted epitopes with respect to positions along a p22 consensus sequence; FIG. 305B depicts conservation scores determined for amino acids located at positions along a p22 consensus sequence.

FIG. 306 depicts exemplary sequence analyses performed on amino acid sequences encoded by NTPase of the Norovirus GIX genome. FIG. 306A shows an identification of exemplary predicted epitopes with respect to positions along an NTPase consensus sequence; FIG. 306B depicts conservation scores determined for amino acids located at positions along an NTPase consensus sequence.

FIG. 307 depicts exemplary sequence analyses performed on amino acid sequences encoded by Nterm of the Norovirus GIX genome. FIG. 307A shows an identification of exemplary predicted epitopes with respect to positions along an Nterm consensus sequence; FIG. 307B depicts conservation scores determined for amino acids located at positions along an Nterm consensus sequence.

FIG. 308 depicts exemplary sequence analyses performed on amino acid sequences encoded by Pro of the Norovirus GIX genome. FIG. 308A shows an identification of exemplary predicted epitopes with respect to positions along a Pro consensus sequence; FIG. 308B depicts conservation scores determined for amino acids located at positions along a Pro consensus sequence.

FIG. 309 depicts exemplary sequence analyses performed on amino acid sequences encoded by RdRp of the Norovirus GIX genome. FIG. 309A shows an identification of exemplary predicted epitopes with respect to positions along an RdRp consensus sequence; FIG. 309B depicts conservation scores determined for amino acids located at positions along an RdRp consensus sequence.

FIG. 310 depicts exemplary sequence analyses performed on amino acid sequences encoded by VP1 of the Norovirus GIX genome. FIG. 310A shows an identification of exemplary predicted epitopes with respect to positions along a VP1 consensus sequence; FIG. 310B depicts conservation scores determined for amino acids located at positions along a VP1 consensus sequence.

FIG. 311 depicts exemplary sequence analyses performed on amino acid sequences encoded by VP2 of the Norovirus GIX genome. FIG. 311A shows an identification of exemplary predicted epitopes with respect to positions along a VP2 consensus sequence; FIG. 311B depicts conservation scores determined for amino acids located at positions along a VP2 consensus sequence.

FIG. 312 depicts exemplary sequence analyses performed on amino acid sequences encoded by VPg of the Norovirus GIX genome. FIG. 312A shows an identification of exemplary predicted epitopes with respect to positions along a VPg consensus sequence; FIG. 312B depicts conservation scores determined for amino acids located at positions along a VPg consensus sequence.

FIG. 313 depicts exemplary sequence analyses performed on amino acid sequences encoded by p22 of the Norovirus GIX genome. FIG. 313A shows an identification of exemplary predicted epitopes with respect to positions along a p22 consensus sequence; FIG. 313B depicts conservation scores determined for amino acids located at positions along a p22 consensus sequence.

FIG. 314 has been modified from van Loben Sels & Green, Viruses 11:432, 2019, which is incorporated herein by reference in its entirety. FIG. 314 includes a schematic of the organization of the human norovirus genome. ORF1 (green) encodes the nonstructural proteins, ORF2 (purple) encodes the major structural capsid protein, VP1, and ORF3 (blue), encodes the minor structural protein, VP2. Amino acids are numbered according to a representative GI.1 genome (GenBank: KF429765.1). The VP1 protein is divided into two major domains: Shell (S) and Protruding (P). The S domain is immediately preceded by a short N-terminal arm (NTA), and the S and P domains are connected by a flexible hinge region (H). The P domain is further subdivided into P1 and P2.

FIG. 315 has been modified from Hassan, E. & Baldridge, M. T, Mucosal Immunology, 12, 1259-1267 (2019), which is incorporated herein by reference in its entirety. FIG. 315 depicts includes a schematic of replication cycle of noroviruses. The replication cycle of NoV begins with attachment (1) of the virus to carbohydrates on the cell surface, where human norovirus (HNoV) binds histo-blood group antigens (HBGAs) and murine norovirus (MNoV) binds other carbohydrates including sialic acids. The proteinaceous receptor is currently only known for MNoVs, which utilize the CD300lf molecule (2), enabling virus entry and uncoating (3) into the host cell. The positive sense RNA genome is then exposed in the cytoplasm, bound at its 5′ end to viral protein VPg. VPg recruits and engages host translation factors, leading to translation (4) of a large polyprotein of at least six non-structural (NS) viral proteins in addition to structural proteins VP1 and VP2, and in the case of MNoV, a virus immune evasion factor VF1 that is produced from an additional open-reading frame (not shown). NS6 (protease) cleaves the viral polyprotein into distinct viral proteins, and host caspases further cleave NS1/2 into NS1 and NS2. The viral RNA-dependent RNA polymerase then engages viral +RNA to start transcription and replication of the virus genome (5). Typical for RNA viruses, replication ensues through a −RNA replication intermediate that serves as a template to produce new viral +RNA genomes. Viral structural proteins then combine with nascent viral +RNA molecules for assembly (6) of new virus particles that exit the cell (7) through yet-to-be-discovered mechanisms.

FIG. 316 has been reproduced from van Loben Sels & Green, Viruses 11:432, 2019, which is incorporated herein by reference in its entirety. FIG. 316 illustrates a comparison of histo-blood group antigen (HBGA) blockade epitopes mapped to (Panel A) GI.1, (Panel B) GII.4, and (Panel C) GI.10 P domains. HBGA binding residues are denoted in gold above the linear representation and the three-dimensional models of human NoV P domains. Adjacent tables record epitope specificities and coloring that corresponds to the positions of the amino acids on both the linear and three-dimensional diagrams. Antibodies marked with an asterisk (*) do not have an antibody/virus structure associated with their epitope definition. ChimeraX was used to model amino acid binding sites (GI.1 PDB 2ZL6, GII.4 PDB 20BS, GII.10 PDB 3ONU).

FIG. 317 depicts an optional immunization protocol for mouse studies.

FIG. 318 presents and exemplary workflow for identification, selection and/or characterization of antigens (e.g., CMV proteins, including particular variants, and/or epitopes thereof, in particular T cell epitopes) for use in accordance with the present disclosure.

### DEFINITIONS

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Agent: As used herein, the term “agent”, may refer to a physical entity or phenomenon. In some embodiments, an agent may be characterized by a particular feature and/or effect. In some embodiments, an agent may be a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amino acid: In its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Antibody agent: As used herein, the term “antibody agent” refers to an agent that specifically binds to a particular antigen. In some embodiments, the term encompasses a polypeptide or polypeptide complex that includes immunoglobulin structural elements sufficient to confer specific binding. For example, in some embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent in or comprises a polypeptide whose amino acid sequence includes structural elements recognized by those skilled in the art to correspond to CDRs1, 2, and 3 of an antibody variable domain; in some such embodiments, an antibody agent in or comprises a polypeptide or set of polypeptides whose amino acid sequence(s) together include structural elements recognized by those skilled in the art to correspond to both heavy chain and light chain variable region CDRs, e.g., heavy chain CDRs 1, 2, and/or 3 and light chain CDRs 1, 2, and/or 3. In some embodiments, an antibody agent is a polypeptide protein having a binding domain which is homologous or largely homologous to an immunoglobulin-binding domain. In some embodiments, an antibody agent may be or comprise a polyclonal antibody preparation. In some embodiments, an antibody agent may be or comprise a monoclonal antibody preparation. In some embodiments, an antibody agent may include one or more constant region sequences that are characteristic of a particular organism, such as a camel, human, mouse, primate, rabbit, rat; in many embodiments, an antibody agent may include one or more constant region sequences that are characteristic of a human. In some embodiments, an antibody agent may include one or more sequence elements that would be recognized by one skilled in the art as a humanized sequence, a primatized sequence, a chimeric sequence, etc. In some embodiments, an antibody agent may be a canonical antibody (e.g., may comprise two heavy chains and two light chains). In some embodiments, an antibody agent may be in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.].

Antigen: Those skilled in the art, reading the present specification, will appreciate that the term “antigen” refers to a molecule that is recognized by the immune system, e.g., in particular embodiments the adaptive immune system, such that it elicits an antigen-specific immune response. In some embodiments, an antigen-specific immune response may be or comprise generation of antibodies and/or antigen-specific T cells. In some embodiments, an antigen is a peptide or polypeptide that comprises at least one epitope against which an immune response can be generated. In one embodiment, an antigen is presented by cells of the immune system such as antigen presenting cells like dendritic cells or macrophages. In one embodiments, an antigen or a processed product thereof such as a T-cell epitope is bound by a T- or B-cell receptor, or by an immunoglobulin molecule such as an antibody. Accordingly, an antigen or a processed product thereof may react specifically with antibodies or T lymphocytes (T cells). In one embodiment, an antigen is a viral antigen. In accordance with the present disclosure, in some embodiments, an antigen may be delivered by RNA molecules as described herein. In some embodiments, a peptide or polypeptide antigen can be 2-100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide or polypeptide antigen can be greater than 50 amino acids. In some embodiments, a peptide or polypeptide antigen can be greater than 100 amino acids. In some embodiments, an antigen is recognized by an immune effector cell. In some embodiments, an antigen if recognized by an immune effector cell is able to induce in the presence of appropriate co-stimulatory signals, stimulation, priming and/or expansion of the immune effector cell carrying an antigen receptor recognizing the antigen. In the context of the embodiments of the present disclosure, in some embodiments, an antigen can be presented or present on the surface of a cell, e.g., an antigen presenting cell. In one embodiment, an antigen is presented by a diseased cell such as a virus-infected cell. In one embodiment, an antigen receptor is a TCR which binds to an epitope of an antigen presented in the context of MHC. In one embodiment, binding of a TCR when expressed by T cells and/or present on T cells to an antigen presented by cells such as antigen presenting cells results in stimulation, priming and/or expansion of said T cells. In one embodiment, binding of a TCR when expressed by T cells and/or present on T cells to an antigen presented on diseased cells results in cytolysis and/or apoptosis of the diseased cells, wherein said T cells preferably release cytotoxic factors, e.g. perforins and granzymes.

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of, susceptibility to, severity of, stage of, etc the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: Those skilled in the art, reading the present specification, will appreciate that the term “binding” typically refers to a non-covalent association between or among entities or moieties. In some embodiments, binding data are expressed in terms of “IC50”. As is understood in the art, IC50 is the concentration of an assessed agent in a binding assay at which 50% inhibition of binding of reference agent known to bind the relevant binding partner is observed. In some embodiments, assays are run under conditions in which the assays are run (e.g., limiting binding target and reference concentrations), these values approximate K_{D} values. Assays for determining binding are well known in the art and are described in detail, for example, in PCT publications WO 94/20127 and WO 94/03205, and other publications such Sidney et al., Current Protocols in Immunology 18.3.1 (1998); Sidney, et al., J. Immunol. 154:247 (1995); and Sette, et al., Mol. Immunol. 31:813 (1994). Alternatively, binding can be expressed relative to binding by a reference standard peptide. For example, can be based on its IC₅₀, relative to the IC₅₀ of a reference standard peptide. Binding can also be determined using other assay systems including those using: live cells (e.g., Ceppellini et al., Nature 339:392 (1989); Christnick et al., Nature 352:67 (1991); Busch et al., it. Immunol. 2:443 (1990); Hill et al., J. Immunol. 147:189 (1991); del Guercio et al., J. Immunol. 154:685 (1995)), cell free systems using detergent lysates (e.g., Cerundolo et al., J. Immunol 21:2069 (1991)), immobilized purified MHC (e.g., Hill et al., J. Immunol. 152, 2890 (1994); Marshall et al., J. Immunol. 152:4946 (1994)), ELISA systems (e.g., Reay et al., EMBO J. 11:2829 (1992)), surface plasmon resonance (e.g., Khilko et al., J. Biol. Chem. 268:15425 (1993)); high flux soluble phase assays (Hammer et al., J. Exp. Med. 180:2353 (1994)), and measurement of class I MHC stabilization or assembly (e.g., Ljunggren et al., Nature 346:476 (1990); Schumacher et al., Cell 62:563 (1990); Townsend et al., Cell 62:285 (1990); Parker et al., J. Immunol. 149:1896 (1992)).

Cap: As used herein, the term “cap” refers to a structure comprising or essentially consisting of a nucleoside-5′-triphosphate that is typically joined to a 5′-end of an uncapped RNA (e.g., an uncapped RNA having a 5′-diphosphate). In some embodiments, a cap is or comprises a guanine nucleotide. In some embodiments, a cap is or comprises a naturally-occurring RNA 5′ cap, including, e.g., but not limited to a 7-methylguanosine cap, which has a structure designated as “m7G.” In some embodiments, a cap is or comprises a synthetic cap analog that resembles an RNA cap structure and possesses the ability to stabilize RNA if attached thereto, including, e.g., but not limited to anti-reverse cap analogs (ARCAs) known in the art). Those skilled in the art will appreciate that methods for joining a cap to a 5′ end of an RNA are known in the art. For example, in some embodiments, a capped RNA may be obtained by in vitro capping of RNA that has a 5′ triphosphate group or RNA that has a 5′ diphosphate group with a capping enzyme system (including, e.g., but not limited to vaccinia capping enzyme system or Saccharomyces cerevisiae capping enzyme system). Alternatively, a capped RNA can be obtained by in vitro transcription (IVT) of a single-stranded DNA template in the presence of a dinucleotide or trinucleotide cap analog.

Cell-mediated immunity: “Cell-mediated immunity”, “cellular immunity”, “cellular immune response”, or similar terms are meant to include a cellular response directed to cells characterized by expression of an antigen, in particular characterized by presentation of an antigen with class I or class II MHC. A cellular response relates to immune effector cells, in particular to T cells or T lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as virus-infected cells, preventing the production of more diseased cells.

Co-administration: As used herein, the term “co-administration” refers to use of a pharmaceutical composition (e.g., immunogenic composition, e.g., vaccine) described herein and an additional therapeutic agent. The combined use of a pharmaceutical composition (e.g., immunogenic composition, e.g., vaccine) described herein and an additional therapeutic agent may be performed concurrently or separately (e.g., sequentially in any order). In some embodiments, a pharmaceutical composition (e.g., immunogenic composition, e.g., vaccine) described herein and an additional therapeutic agent may be combined in one pharmaceutically-acceptable carrier, or they may be placed in separate carriers and delivered to a target cell or administered to a subject at different times. Each of these situations is contemplated as falling within the meaning of “co-administration” or “combination,” provided that a pharmaceutical composition (e.g., immunogenic composition, e.g., vaccine) described herein and an additional therapeutic agent are delivered or administered sufficiently close in time that there is at least some temporal overlap in biological effect(s) generated by each on a target cell or a subject being treated.

Codon-optimized: As used herein, the term “codon-optimized” refers to alteration of codons in a coding region of a nucleic acid molecule to reflect the typical codon usage of a host organism without preferably altering the amino acid sequence encoded by the nucleic acid molecule. Within the context of the present disclosure, in some embodiments coding regions are codon-optimized for optimal expression in a subject to be treated using the RNA molecules described herein. In some embodiments, codon-optimization may be performed such that codons for which frequently occurring tRNAs are available are inserted in place of “rare codons”. In some embodiments, codon-optimization may include increasing guanosine/cytosine (G/C) content of a coding region of RNA described herein as compared to the G/C content of the corresponding coding sequence of a wild type RNA, wherein the amino acid sequence encoded by the RNA is preferably not modified compared to the amino acid sequence.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190′ amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.

Derived: In the context of an amino acid sequence (peptide or polypeptide) “derived from” a designated amino acid sequence (peptide or polypeptide), it refers to a structural analogue of a designated amino acid sequence. In some embodiments, an amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

Designed: As used herein, the term “designed” refers to an agent (i) whose structure is or was selected by the hand of man; (ii) that is produced by a process requiring the hand of man; and/or (iii) that is distinct from natural substances and other known agents.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Encode: As used herein, the term “encode” or “encoding” refers to sequence information of a first molecule that guides production of a second molecule having a defined sequence of nucleotides (e.g., mRNA) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or an RNA molecule (e.g., an mRNA) encodes a polypeptide if transcription and translation of mRNA corresponding to that gene produces the polypeptide in a cell or other biological system. In some embodiments, a coding region of an RNA molecule encoding a target-antigen refers to a coding strand, the nucleotide sequence of which is identical to the mRNA sequence of such a target antigen. In some embodiments, a coding region of an RNA molecule encoding a target antigen refers to a non-coding strand of such a target antigen, which may be used as a template for transcription of a gene or cDNA.

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

Epitope: As used herein, the term “epitope” refers to a moiety that is specifically recognized by an immunoglobulin (e.g., antibody or receptor) binding component. For example, an epitope may be recognized by a T cell, a B cell, or an antibody. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized). Accordingly, in some embodiments, an epitope of an antigen may include a continuous or discontinuous portion of the antigen. In some embodiments, an epitope is or comprises a T cell epitope. In some embodiments, an epitope may have a length of about 5 to about 30 amino acids, or about 10 to about 25 amino acids, or about 5 to about 15 amino acids, or about 5 to 12 amino acids, or about 6 to about 9 amino acids.

Expression: As used herein, the term “expression” of a nucleic acid sequence refers to the generation of a gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc); (3) translation of an RNA into a polypeptide or protein; and/or (4) post-translational modification of a polypeptide or protein.

Five prime untranslated region: As used herein, the terms “five prime untranslated region” or “5′ UTR” refer to a sequence of an mRNA molecule between a transcription start site and a start codon of a coding region of an RNA. In some embodiments, “5′ UTR” refers to a sequence of an mRNA molecule that begins at a transcription start site and ends one nucleotide (nt) before a start codon (usually AUG) of a coding region of an RNA molecule, e.g., in its natural context.

Fragment: The term “fragment” as used herein in the context of a nucleic acid sequence (e.g. RNA sequence) or an amino acid sequence may typically be a portion of a reference sequence. In some embodiments, a reference sequence is a full-length sequence of e.g. a nucleic acid sequence or an amino acid sequence. Accordingly, a fragment, typically, refers to a sequence that is identical to a corresponding stretch within a reference sequence. In some embodiments, a fragment comprises a continuous stretch of nucleotides or amino acid residues that corresponds to at least 10%, 20%, 30%, 40%, 50%, 60%, 70%, 80%, 90%, 95% of the total length of a reference sequence from which the fragment is derived. In some embodiments, the term “fragment”, with reference to an amino acid sequence (peptide or polypeptide), relates to a part of an amino acid sequence, e.g., a sequence which represents the amino acid sequence shortened at the N-terminus and/or C-terminus. In some embodiments, a fragment of an amino acid sequence comprises at least 6, in particular at least 8, at least 12, at least 15, at least 20, at least 30, at least 50, or at least 100 consecutive amino acids from an amino acid sequence.

Homology: As used herein, the term “homology” or “homolog” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “homologous” to one another if their sequences are at least 15%, 20%, 25%, 30%, 35%, 40%, 45%, 50%, 55%, 60%, 65%, 70%, 75%, 80%, 85%, 90%, 95%, or 99% identical. In some embodiments, polynucleotide molecules (e.g., DNA molecules and/or RNA molecules) and/or polypeptide molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar”side chains. Substitution of one amino acid for another of the same type may often be considered a “homologous” substitution.

Humoral immunity: As used herein, the term “humoral immunity” or “humoral immune response” refers to antibody production and the accessory processes that accompany it, including: Th2 activation and cytokine production, germinal center formation and isotype switching, affinity maturation and memory cell generation. It also refers to the effector functions of antibodies, which include pathogen neutralization, classical complement activation, and opsonin promotion of phagocytosis and pathogen elimination.

Identity: As used herein, the term “identity” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or between polypeptide molecules are considered to be “substantially identical” to one another if their sequences are at least 25%, 30%, 35%, 40%, 45%, 50%, 55%, 60%, 65%, 70%, 75%, 80%, 85%, 90%, 95%, 96%, 97%, 98%, or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Immunologically equivalent: The term “immunologically equivalent” means that an immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, in some embodiments, the term “immunologically equivalent” is used with respect to the immunological effects or properties of antigens or antigen variants used for immunization. For example, an amino acid sequence is immunologically equivalent to a reference amino acid sequence if said amino acid sequence when exposed to the immune system of a subject induces an immune reaction having a specificity of reacting with the reference amino acid sequence.

In one embodiment, an antigen receptor is an antibody or B cell receptor which binds to an epitope in an antigen. In one embodiment, an antibody or B cell receptor binds to native epitopes of an antigen.

Increased, Induced, or Reduced: As used herein, these terms or grammatically comparable comparative terms, indicate values that are relative to a comparable reference measurement. For example, in some embodiments, an assessed value achieved with a provided pharmaceutical composition (e.g., immunogenic composition, e.g., vaccine) may be “increased” relative to that obtained with a comparable reference pharmaceutical composition (e.g., immunogenic composition, e.g., vaccine). Alternatively or additionally, in some embodiments, an assessed value achieved in a subject may be “increased” relative to that obtained in the same subject under different conditions (e.g., prior to or after an event; or presence or absence of an event such as administration of a pharmaceutical composition (e.g., immunogenic composition, e.g., vaccine) as described herein, or in a different, comparable subject (e.g., in a comparable subject that differs from the subject of interest in prior exposure to a condition, e.g., absence of administration of a pharmaceutical composition (e.g., immunogenic composition, e.g., vaccine) as described herein.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance. In some embodiments, the term “reduced” or equivalent terms refers to a reduction in the level of an assessed value by at least 5%, at least 10%, at least 20%, at least 50%, at least 75% or higher, as compared to a comparable reference. In some embodiments, the term “reduced” or equivalent terms refers to a complete or essentially complete inhibition, i.e., a reduction to zero or essentially to zero. In some embodiments, the term “increased” or “induced” refers to an increase in the level of an assessed value by at least 10%, at least 20%, at least 30%, at least 40%, at least 50%, at least 80%, at least 100%, at least 200%, at least 500%, or higher, as compared to a comparable reference.

Ionizable: The term “ionizable” refers to a compound or group or atom that is charged at a certain pH. In the context of an ionizable amino lipid, such a lipid or a function group or atom thereof bears a positive charge at a certain pH. In some embodiments, an ionizable amino lipid is positively charged at an acidic pH. In some embodiments, an ionizable amino lipid is predominately neutral at physiological pH values, e.g., in some embodiments about 7.0-7.4, but becomes positively charged at lower pH values. In some embodiments, an ionizable amino lipid may have a pKa within a range of about 5 to about 7.

Isolated: The term “Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

Lipid: As used herein, the terms “lipid” and “lipid-like material” are broadly defined as molecules which comprise one or more hydrophobic moieties or groups and optionally also one or more hydrophilic moieties or groups. Molecules comprising hydrophobic moieties and hydrophilic moieties are also typically denoted as amphiphiles.

RNA lipid nanoparticle: As used herein, the term “RNA lipid nanoparticle” refers to a nanoparticle comprising at least one lipid and RNA molecule(s). In some embodiments, an RNA lipid nanoparticle comprises at least one ionizable amino lipid. In some embodiments, an RNA lipid nanoparticle comprises at least one ionizable amino lipid, at least one helper lipid, and at least one polymer-conjugated lipid (e.g., PEG-conjugated lipid). In various embodiments, RNA lipid nanoparticles as described herein can have an average size (e.g., Z-average) of about 100 nm to 1000 nm, or about 200 nm to 900 nm, or about 200 nm to 800 nm, or about 250 nm to about 700 nm. In some embodiments of the present disclosure, RNA lipid nanoparticles can have a particle size (e.g., Z-average) of about 30 nm to about 200 nm, or about 30 nm to about 150 nm, about 40 nm to about 150 nm, about 50 nm to about 150 nm, about 60 nm to about 130 nm, about 70 nm to about 110 nm, about 70 nm to about 100 nm, about 80 nm to about 100 nm, about 90 nm to about 100 nm, about 70 to about 90 nm, about 80 nm to about 90 nm, or about 70 nm to about 80 nm. In some embodiments, an average size of lipid nanoparticles is determined by measuring the particle diameter. In some embodiments, RNA lipid nanoparticles may be prepared by mixing lipids with RNA molecules described herein.

Lipidoid: As used herein, a lipidoid refers to a lipid-like molecule. In some embodiments, a lipoid is an amphiphilic molecule with one or more lipid-like physical properties. In the context of the present disclosure, the term lipid is considered to encompass lipidoids.

Nanoparticle: As used herein, the term “nanoparticle” refers to a particle having an average size suitable for parenteral administration. In some embodiments, a nanoparticle has a longest dimension (e.g., a diameter) of less than 1,000 nanometers (nm). In some embodiments, a nanoparticle may be characterized by a longest dimension (e.g., a diameter) of less than 300 nm. In some embodiments, a nanoparticle may be characterized by a longest dimension (e.g., a diameter) of less than 100 nm. In many embodiments, a nanoparticle may be characterized by a longest dimension between about 1 nm and about 100 nm, or between about 1 μm and about 500 nm, or between about 1 nm and 1,000 nm. In many embodiments, a population of nanoparticles is characterized by an average size (e.g., longest dimension) that is below about 1,000 nm, about 500 nm, about 100 nm, about 50 nm, about 40 nm, about 30 nm, about 20 nm, or about 10 nm and often above about 1 nm. In many embodiments, a nanoparticle may be substantially spherical so that its longest dimension may be its diameter. In some embodiments, a nanoparticle has a diameter of less than 100 nm as defined by the National Institutes of Health.

Naturally occurring: The term “naturally occurring” as used herein refers to an entity that can be found in nature. For example, a peptide or nucleic acid that is present in an organism (including viruses) and can be isolated from a source in nature and which has not been intentionally modified by man in the laboratory is naturally occurring.

Neutralization: As used herein, the term “neutralization” refers to an event in which binding agents such as antibodies bind to a biological active site of a virus such as a receptor binding protein, thereby inhibiting the parasitic infection of cells. In some embodiments, the term “neutralization” refers to an event in which binding agents eliminate or significantly reduce ability of infecting cells.

Nucleic acid particle: A “nucleic acid particle” can be used to deliver nucleic acid to a target site of interest (e.g., cell, tissue, organ, and the like). A nucleic acid particle may comprise at least one cationic or cationically ionizable lipid or lipid-like material, at least one cationic polymer such as protamine, or a mixture thereof and nucleic acid. In some embodiments, a nucleic acid particle is a lipid nanoparticle. In some embodiments, a nucleic acid particle is a lipoplex particle.

Nucleic acid/Polynucleotide: As used herein, the term “nucleic acid” refers to a polymer of at least 10 nucleotides or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Nucleotide: As used herein, the term “nucleotide” refers to its art-recognized meaning. When a number of nucleotides is used as an indication of size, e.g., of a polynucleotide, a certain number of nucleotides refers to the number of nucleotides on a single strand, e.g., of a polynucleotide.

Patient: As used herein, the term “patient” refers to any organism who is suffering or at risk of a disease or disorder or condition. Typical patients include animals (e.g., mammals such as mice, rats, rabbits, non-human primates, and/or humans). In some embodiments, a patient is a human. In some embodiments, a patient is suffering from or susceptible to one or more diseases or disorders or conditions. In some embodiments, a patient displays one or more symptoms of a disease or disorder or condition. In some embodiments, a patient has been diagnosed with one or more diseases or disorders or conditions. In some embodiments, a disease or disorder or condition that is amenable to provided technologies is or includes a viral infection. In some embodiments, a patient is receiving or has received certain therapy to diagnose and/or to treat a disease, disorder, or condition. In some embodiments, a patient is a patient suffering from or susceptible to a viral infection.

PEG-conjugated lipid: The term “PEG-conjugated lipid” refers to a molecule comprising a lipid portion and a polyethylene glycol portion.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for parenteral administration, for example, by subcutaneous, intramuscular, or intravenous injection as, for example, a sterile solution or suspension formulation.

Pharmaceutically effective amount: The term “pharmaceutically effective amount” or “therapeutically effective amount” refers to the amount which achieves a desired reaction or a desired effect alone or together with further doses. In the case of the treatment of a particular disease, a desired reaction in some embodiments relates to inhibition of the course of the disease. In some embodiments, such inhibition may comprise slowing down the progress of a disease and/or interrupting or reversing the progress of the disease. In some embodiments, a desired reaction in a treatment of a disease may be or comprise delay or prevention of the onset of a disease or a condition. An effective amount of pharmaceutical compositions (e.g., immunogenic compositions, e.g., vaccines) described herein will depend, for example, on a disease or condition to be treated, the severity of such a disease or condition, individual parameters of the patient, including, e.g., age, physiological condition, size and weight, the duration of treatment, the type of an accompanying therapy (if present), the specific route of administration and similar factors. Accordingly, doses of pharmaceutical compositions (e.g., immunogenic compositions, e.g., vaccines) described herein may depend on various of such parameters. In the case that a reaction in a patient is insufficient with an initial dose, higher doses (or effectively higher doses achieved by a different, more localized route of administration) may be used.

Poly(A) sequence: As used herein, the term “poly(A) sequence” or “poly-A tail” refers to an uninterrupted or interrupted sequence of adenylate residues which is typically located at the 3′-end of an RNA molecule. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. RNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

Polypeptide: As used herein, the term “polypeptide” refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide.

Prevent: As used herein, the term “prevent” or “prevention” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Recombinant: The term “recombinant” in the context of the present disclosure means “made through genetic engineering”. In some embodiments, a “recombinant” entity such as a recombinant nucleic acid in the context of the present disclosure is not naturally occurring.

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Ribonucleic acid (RNA): As used herein, the term “RNA” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments where an RNA is a mRNA. In some embodiments where an RNA is a mRNA, a RNA typically comprises at its 3′ end a poly(A) region. In some embodiments where an RNA is a mRNA, an RNA typically comprises at its 5′ end an art-recognized cap structure, e.g., for recognizing and attachment of a mRNA to a ribosome to initiate translation. In some embodiments, a RNA is a synthetic RNA. Synthetic RNAs include RNAs that are synthesized in vitro (e.g., by enzymatic synthesis methods and/or by chemical synthesis methods).

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Risk: As will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more. In some embodiments, risk may reflect one or more genetic attributes, e.g., which may predispose an individual toward development (or not) of a particular disease, disorder and/or condition. In some embodiments, risk may reflect one or more epigenetic events or attributes and/or one or more lifestyle or environmental events or attributes.

RNA lipoplex particle: As used herein, the term “RNA lipoplex particle” refers to a complex comprising liposomes, in particular cationic liposomes, and RNA molecules. Without wishing to bound by a particular theory, electrostatic interactions between positively charged liposomes and negatively charged RNA results in complexation and spontaneous formation of RNA lipoplex particles. In some embodiments, positively charged liposomes may comprise a cationic lipid, such as in some embodiments DOTMA, and additional lipids, such as in some embodiments DOPE. In one embodiment, a RNA lipoplex particle is a nanoparticle.

Selective or specific: The term “selective” or “specific”, when used herein in reference to an agent having an activity, is understood by those skilled in the art to mean that the agent discriminates between potential target entities, states, or cells. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of a target-binding moiety for one or more other potential target entities (e.g., competitors). In some embodiments, specificity is evaluated relative to that of a reference specific binding moiety. In some embodiments, specificity is evaluated relative to that of a reference non-specific binding moiety.

Stable: As used herein, the term “stable” in the context of the present disclosure refers to a pharmaceutical composition (e.g., immunogenic composition, e.g., vaccine) as a whole and/or components thereof meeting or exceeding pre-determined acceptance criteria. For example, in some embodiments, a stable pharmaceutical composition (e.g., immunogenic composition, e.g., vaccine) exhibits no unacceptable levels of microbial growth, and substantially no or no breakdown or degradation of the active biological molecule component(s). In some embodiments, a stable pharmaceutical composition (e.g., immunogenic composition, e.g., vaccine) refers to the integrity of RNA molecules being maintained at least above 90% or more. In some embodiments, a stable pharmaceutical composition (e.g., immunogenic composition, e.g., vaccine) refers to at least 90% or more (including, e.g., at least 95%, at least 96%, at least 97%, or more) of RNA molecules being maintained to be encapsulated within lipid nanoparticles. In some embodiments, a stable pharmaceutical composition (e.g., immunogenic composition, e.g., vaccine) refers to a formulation that remains capable of eliciting a desired immunologic response when administered to a subject. In some embodiments, a pharmaceutical composition (e.g., immunogenic composition, e.g., vaccine) remains stable for a specified period of time under certain conditions.

Subject: As used herein, the term “subject” refers to an organism to be administered with a composition described herein, e.g., for experimental, diagnostic, prophylactic, and/or therapeutic purposes. Typical subjects include animals (e.g., mammals such as mice, rats, rabbits, non-human primates, domestic pets, etc.) and humans. In some embodiments, a subject is a human subject. In some embodiments, a subject is suffering from a disease, disorder, or condition (e.g., viral infection). In some embodiments, a subject is susceptible to a disease, disorder, or condition (e.g., viral infection). In some embodiments, a subject displays one or more symptoms or characteristics of a disease, disorder, or condition (e.g., viral infection). In some embodiments, a subject displays one or more non-specific symptoms of a disease, disorder, or condition (e.g., viral infection). In some embodiments, a subject does not display any symptom or characteristic of a disease, disorder, or condition (e.g., viral infection). In some embodiments, a subject is someone with one or more features characteristic of susceptibility to or risk of a disease, disorder, or condition (e.g., viral infection). In some embodiments, a subject is a patient. In some embodiments, a subject is an individual to whom diagnosis and/or therapy is and/or has been administered.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: As used herein, the term “synthetic” refers to an entity that is artificial, or that is made with human intervention, or that results from synthesis rather than naturally occurring. For example, in some embodiments, a synthetic nucleic acid or polynucleotide refers to a nucleic acid molecule that is chemically synthesized, e.g., in some embodiments by solid-phase synthesis. In some embodiments, the term “synthetic” refers to an entity that is made outside of biological cells. For example, in some embodiments, a synthetic nucleic acid or polynucleotide refers to a nucleic acid molecule (e.g., an RNA) that is produced by in vitro transcription using a template.

Therapy: The term “therapy” refers to an administration or delivery of an agent or intervention that has a therapeutic effect and/or elicits a desired biological and/or pharmacological effect (e.g., has been demonstrated to be statistically likely to have such effect when administered to a relevant population). In some embodiments, a therapeutic agent or therapy is any substance that can be used to alleviate, ameliorate, relieve, inhibit, prevent, delay onset of, reduce severity of, and/or reduce incidence of one or more symptoms or features of a disease, disorder, and/or condition. In some embodiments, a therapeutic agent or therapy is a medical intervention (e.g., surgery, radiation, phototherapy) that can be performed to alleviate, relieve, inhibit, present, delay onset of, reduce severity of, and/or reduce incidence of one or more symptoms or features of a disease, disorder, and/or condition.

Three prime untranslated region: As used herein, the terms “three prime untranslated region” or “3′ UTR” refer to a sequence of an mRNA molecule that begins following a stop codon of a coding region of an open reading frame sequence. In some embodiments, the 3′ UTR begins immediately after a stop codon of a coding region of an open reading frame sequence, e.g., in its natural context. In other embodiments, the 3′ UTR does not begin immediately after stop codon of the coding region of an open reading frame sequence, e.g., in its natural context.

Threshold level (e.g., acceptance criteria): As used herein, the term “threshold level” refers to a level that are used as a reference to attain information on and/or classify the results of a measurement, for example, the results of a measurement attained in an assay. For example, in some embodiments, a threshold level means a value measured in an assay that defines the dividing line between two subsets of a population (e.g. a batch that satisfy quality control criteria vs. a batch that does not satisfy quality control criteria). Thus, a value that is equal to or higher than the threshold level defines one subset of the population, and a value that is lower than the threshold level defines the other subset of the population. A threshold level can be determined based on one or more control samples or across a population of control samples. A threshold level can be determined prior to, concurrently with, or after the measurement of interest is taken. In some embodiments, a threshold level can be a range of values.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition. In some embodiments, treatment may be administered to a subject at a later-stage of disease, disorder, and/or condition.

Vaccination: As used herein, the term “vaccination” refers to the administration of a composition intended to generate an immune response, for example to a disease-associated (e.g., disease-causing) agent. In some embodiments, vaccination can be administered before, during, and/or after exposure to a disease-associated agent, and in certain embodiments, before, during, and/or shortly after exposure to the agent. In some embodiments, vaccination includes multiple administrations, appropriately spaced in time, of a vaccine composition. In some embodiments, vaccination generates an immune response to an infectious agent.

Vaccine: As used herein, the term “vaccine” refers to a composition that induces an immune response upon administration to a subject. In some embodiments, an induced immune response provides protective immunity.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature.

Vector: as used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In some embodiments, known techniques may be used, for example, for generation or manipulation of recombinant DNA, for oligonucleotide synthesis, and for tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (4th ed., Cold Spring Harbor Laboratory Press, Cold Spring Harbor, N.Y. (2012)), which is incorporated herein by reference for any purpose.

### DETAILED DESCRIPTION OF CERTAIN EMBODIMENTS

As discussed above, the present disclosure provides pharmaceutical compositions (e.g., immunogenic compositions, e.g., vaccines) for delivering particular viral antigen constructs to a subject (e.g., a patient) and related technologies (e.g., methods). In particular, the present disclosure provides vaccine compositions and related technologies (e.g., methods) for prophylactic or therapeutic treatment for viruses, particularly viruses that experience a latent phase. In some embodiments, viral antigens in a pharmaceutical composition can be antigens from a polypeptide or portion thereof from HSV-1, HSV-2, VZV, Human Immnunodeficiency Virus (HIV), Epstein Barr Virus (EBV), CMV, HHV-6, HHV-7, KSHV (HHV-8), JC virus (JCV), BK virus (BKV), parvovirus, or adenovirus.

In some embodiments, pharmaceutical compositions described herein can include one or more antigens derived from a viral polypeptide that is exposed to serum during the life cycle of a virus. In some embodiments, pharmaceutical compositions described herein can include one or more antigens derived from a viral polypeptide that is expressed during a latent phase of the life cycle of a virus.

Information on certain exemplary pharmaceutical compositions and viruses is included herein.

### Varicella-Zoster Virus (VZV)

VZV, also known as human herpesvirus 3 is an alphaherpesvirus with a single, linear, double-stranded molecule that is about 125,000 nt long. VZV virions are spherical and are approximately 180-200 nm in diameter. VZV virions have a lipid envelope that encloses a 100 nm nucleocapsid of 162 hexameric and pentameric capsomeres arranged in an icosahedral form. A VZV capsid is surrounded by loosely associated proteins, which are collectively referred to as a tegument. Tegument proteins can play a role in, e.g., initiating viral reproduction in an infected cell. A tegument is covered by a lipid envelope studded with glycoproteins that are displayed on the exterior of the virion. VZV is closely related to the herpes simplex viruses (HSV), and VZV's envelope glycoproteins (e.g., gB, gC, gE, gH, gI, gK, gL) correspond with envelope glycoproteins of HSV. Because the envelope glycoproteins are exposed prior to VZV entering cells of a host human, the envelope glycoproteins or fragments of those glycoproteins can be useful as VZV antigens for generating a an immune response to VZV. Further, tegument proteins may also be useful as VZV antigens for generating a an immune response to VZV.

VZV only naturally infects humans, with no known animal reservoir. Within humans, VZV targets include T lymphocytes, epithelial cells, and ganglia. VZV is highly communicable and spreads by the airborne route, with an extraordinarily high transmission rate in temperate countries. Without wishing to be bound by any particular theory, most VZV particles comes from skin, where the virus is highly concentrated in vesicles. Generally, it is believed that skin cells and cell-free VZV are frequently shed and may be a major source of infectious cell-free airborne virus.

### Lifecycle

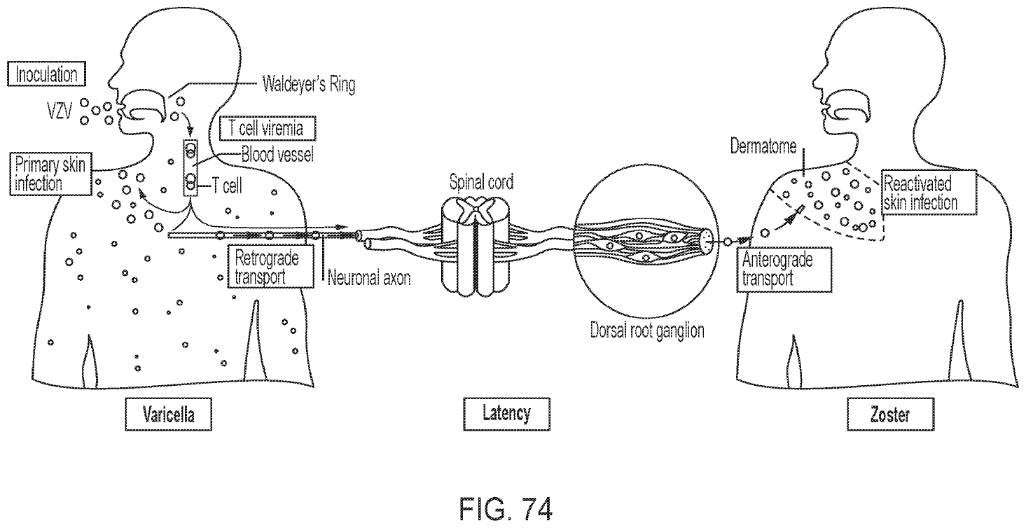

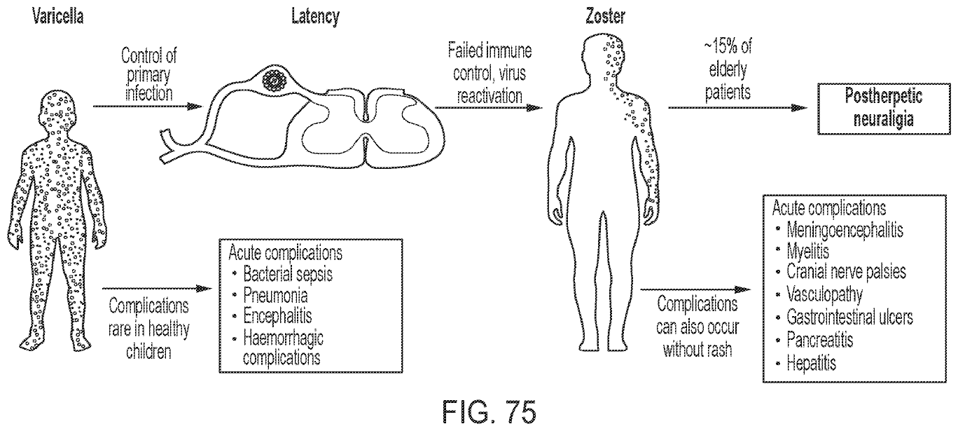

FIGS. 74 and 75 include schematics illustrating various aspects of the VZV life cycle. (Zerboni, L., et al., “Molecular mechanisms of varicella zoster virus pathogenesis,” Nat Rev Microbiol., 12(3):197-210 (March 2014); Gershon, A. A., et al., “Varicella zoster virus infection,” Nat Rev Dis Primers, 2015 Jul. 2; 1:15016 (July 2015), each of which is herein incorporated by reference in its entirety). As shown, generally, VZV infects a human host when virus particles reach mucosal epithelial sites of entry. From the mucosal sites, VZV spreads to the tonsils and other local lymphoid tissues, where the virus can infect T cells. Infected T cells can then transport the VZV via the bloodstream to the skin. It is during this primary infection when VZV presumably gain access to the sensory nerve cell bodies in ganglia by retrograde axonal transport from sites of infection on the skin or in T cells. Latent infection can then be established.

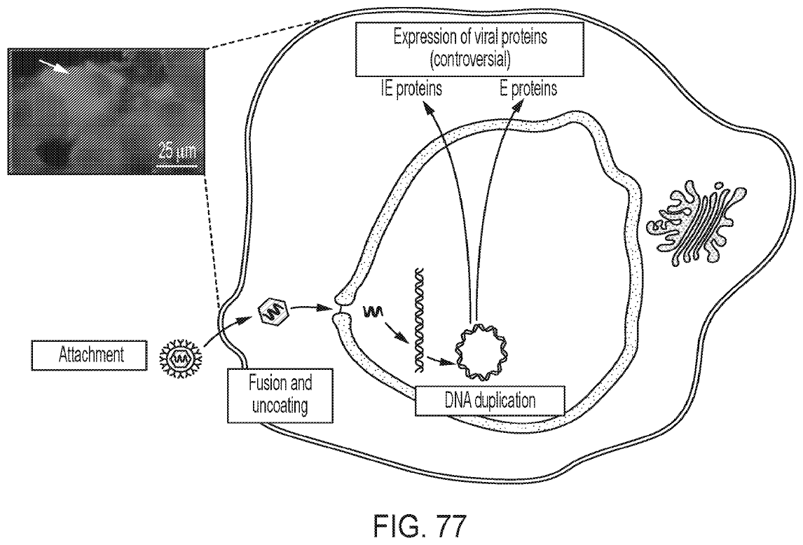

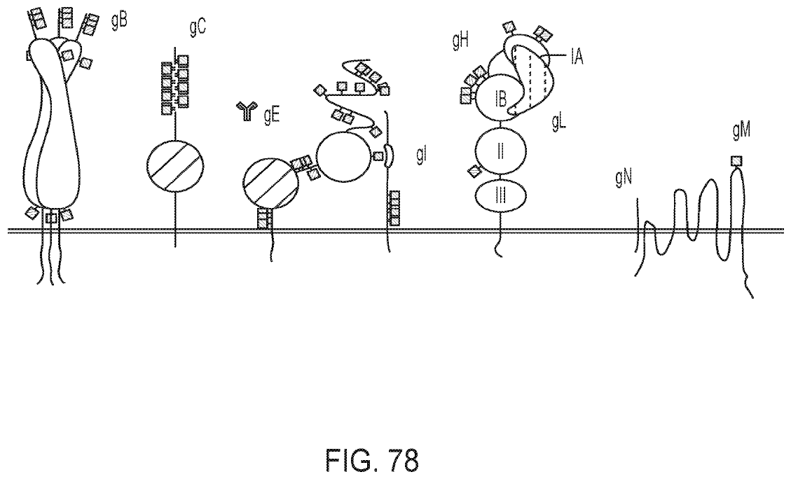

Since most adults in the world (>95%) have developed varicella, they have the complete VZV genome latent in 5% of their neurons (˜5-10 copies/latently infected neuron). Latency is defined by the inability to recover the virus in tissue culture or visualize it by electron microscopy. The exact mechanisms of latent infection remain unclear but, as shown in FIG. 77, viral replication is thought to stop at the circular DNA stage. Accordingly, limited, if any, protein expression is believed to occur, and no viral proteins have been observed in the nucleus during latency. No easily observable changes of cell morphology have been observed during latent infection (see micrograph of guinea pig enteric neurons in top right portion of FIG. 77). Isolated neurons were cultured in vitro and infected with cell-free VZV to induce infection.

Reactivation from latency enables a second phase of replication to occur in skin, which typically causes lesions in the dermatome that is innervated by the affected sensory ganglion. When viral replication is reactivated, VZV reaches the skin via anterograde axonal transport to cause the symptoms of zoster. As discussed above, zoster is often characterized by a vesicular rash in a dermatome that is innervated by the affected ganglion. Both varicella and zoster skin lesions contain high concentrations of infectious virus and are thus responsible for transmission to susceptible individuals.

Latent VZV reactivates intermittently to form infectious virions. The frequency and magnitude of reactivation events are unknown, but the reactivations typically remain subclinical because they are controlled by the VZV-specific immune responses that previously developed with varicella. However, while post-varicella antibody responses remain relatively unchanged lifelong, VZV directed immune responses decline with age. As a result, when latent VZV reactivates in sensory ganglia, and the local immune responses have become inadequate to prevent propagation of the infection, VZV infection will reactivate to cause zoster. The correlation of the age-related decline of VZV-specific immune responses with the age-related increase in zoster frequency and severity suggest that VZV-specific immune responses play a role in preventing zoster. As such, vaccines that can restore VZV-specific immune responses that decline during the aging process would be helpful in suppressing zoster reactivation, which would minimize complications associated with zoster, as well as further spread of VZV. Thus, an effective VZV vaccine remains an unmet medical need of critical importance for global health.

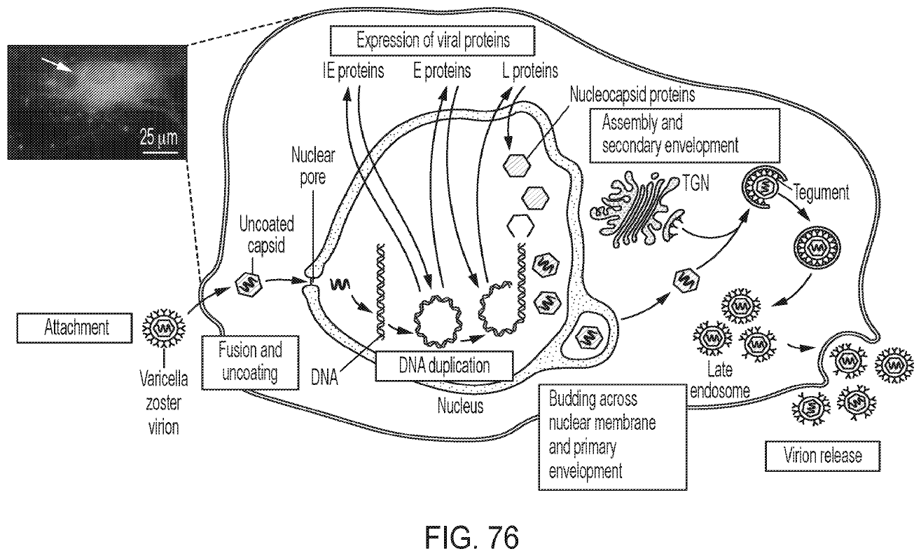

Varicella and zoster infection both involve lytic infection, which includes replication of the VZV genome and production of VZV virions. As shown in FIG. 76, lytic infection of VZV starts with attachment, fusion and uncoating of an infecting virion. The virus capsid is then transported to the cell nucleus, where the VZV DNA becomes circular. A set of viral proteins, including immediate early (IE), early (E) and late (L) proteins, are expressed and enter the nucleus. New virions then bud from the nuclear membrane and undergo secondary envelopment to add tegument and envelope proteins to the virion particles prior to exit from an infected cell. This full cycle of viral replication leads to substantial cell damage and eventually lysis, as shown in the micrograph in the top left of FIG. 76.

### Varicella

As discussed further below, varicella is caused by a primary VZV infection. Varicella occurs worldwide and is endemic in populations of sufficient size to sustain year-round transmission. In fact, on average, epidemics occur every 2-3 years. Varicella epidemiology and disease burden have been studied primarily in developed countries. Characterization of the global health burden of VZV has been hindered by a lack of data. For example, while VZV seroprevalance data are becoming more widely available, additional data are needed on severe disease outcomes and deaths to better characterize the health burden in regions such as Africa and India.

Varicella incidence ranges from 13 to 16 cases per 1,000 persons per year, with substantial yearly variation. Incidence rates are, at least in part, correlated with climate. In temperate climates, varicella incidence is highest in preschool aged children (1-4 years of age) or children in early elementary school (5-9 years of age). The annual incidence in those regions tends to be greater than 100 per 1,000 children. Consequently, greater than 90% of people become infected before adolescence. In tropical climates, acquisition of varicella occurs at a higher overall mean age, with a higher proportion of cases in adults. Without wishing to be bound by any particular theory, differences in varicella epidemiology between temperate and tropical climates might be related to the properties of VZV, for example, inactivation by heat and/or humidity, or factors affecting the risk of exposure. Varicella also shows a strong seasonal pattern, with peak incidence during cool, dry seasons.

Outbreaks occur commonly in settings where people, in particular, children, congregate. However, other settings in which outbreaks comment occur include hospitals, facilities for institutionalized people, refugee camps, military facilities, and correctional facilities.

VZV transmission is highly efficient, and prior to the introduction of preventive therapies (e.g., vaccines), most children were infected with VZV and contract varicella before the age of 10. Complications from varicella in children can be unpredictable, although majority of children do not require any significant medical intervention. In developed countries, ˜5 out of 1,000 people with varicella are hospitalized and 2-3 per 100,000 patients die. Serious varicella complications include bacterial sepsis, pneumonia, encephalitis and hemorrhage. Adults, infants and individuals who are severely immunocompromised are at higher risk of severe complications and death. Varicella acquired in the first two trimesters of pregnancy can also cause severe congenital defects in the newborn. About 1% of affected pregnancies result in babies with severe congenital defects.

Historically, VZV epidemics have been self-limiting because the high rate of transmission and disease-induced immunity deplete the pool of susceptible individuals. However, as with many viruses, the potential for genetic mutations that result in a change in VZV's epidemiology or alter the nature and severity of varicella epidemics remains a concern.

### Herpes Zoster

Herpes zoster, also known as shingles or zoster, is caused by the reactivation of the varicella-zoster virus (VZV), the same virus that causes varicella (colloquially referred to as “chickenpox”). As discussed herein, primary infection with VZV causes varicella. Varicella typically results in the appearance of VZV-specific humoral and T cell mediated immunity. These immune responses drive termination of varicella, and are also important for preventing zoster.

Herpes zoster epidemiology is most frequently observed in developed countries with long life expectancies. In developed countries, for example, by the age of 85 years, greater than 50% of the population reports at least one episode of herpes zoster. The incidence and severity of herpes zoster both tend to increase with age due to declining cell-mediated immunity to VZV.

Chronic pain (i.e., postherpetic neuralgia) is a serious complication of zoster and occurs in ˜15% of cases. Age is the most important risk factor for postherpetic neuralgia and the risk increases rapidly after age 50. Other complications of herpes zoster include, but are not limited to, ophthalmic involvement (herpes zoster ophthalmicus) with acute or chronic ocular sequelae, including vision loss; bacterial superinfection of the lesions, usually due to Staphylococcus aureus and, less commonly, due to group A beta hemolytic streptococcus; cranial and peripheral nerve palsies; and visceral involvement, such as meningoencephalitis, pneumonitis, hepatitis, and acute retinal necrosis. People with compromised or suppressed immune systems are more likely to have complications from herpes zoster. They are also more likely to have a severe, long-lasting rash and develop disseminated herpes zoster (e.g., zoster that occurs in three or more dermatomes).

Before VZV vaccines were available, ˜30% of adults developed herpes zoster. As the number of elderly people, immune-suppressed organ transplant recipients, patients receiving chemotherapy for cancer or autoimmune disease, HIV-infected individuals, and patients with chronic illnesses increase, the incidence of herpes zoster is also expected to increase. Race can be protective factor against herpes zoster. Black adults in the United States and the United Kingdom have a 25-50% lower incidence of zoster compared with white adults.

### VZV Genome

A VZV genome comprises a single, double-stranded DNA molecule that is about 125,000 bp long. Generally, the genome is linear in virions with an unpaired nucleotide at each end. In VZV-infected cells, the ends pair and the genome circularizes.

A wild-type VZV genome includes a unique long region (UL) and a unique short region (US). The UL is typically bounded by terminal long (TRL) and internal long (IRL) repeats. The US is typically bounded by internal short (IRS) and terminal short (TRS) repeats. The VZV genome can be configured in two isomers, e.g., in infected cells. The isomers arise because the US region can orientate either of two directions, while the UL region typically maintains a single orientation.

A wild-type VZV genome also includes five repeat regions. Repeat region 1 (R1) is located in open reading frame (ORF) 11. Repeat region 2 (R2) is located in ORF14 (glycoprotein C). Repeat region 3 (R3) is located in ORF22. Repeat region 4 (R4) is located between ORF62 and the origin of viral replication. Finally, repeat region 5 (R5) is located between ORF 60 and 61. The length of the repeat regions varies among different VZV strains. Accordingly, in some instances, the length of one or more repeat regions can be used to distinguish between VZV strains.

A wild-type VZV genome encodes at least 70 genes. VZV genes can be characterized as being within four categories: (1) genes encoding immediate-early genes, (2) genes encoding replication proteins, (3) genes encoding putative late proteins, and (4) genes encoding glycoproteins. Cohen, J. I., “The Varicella-Zoster Virus Genome,” Curr Top Microbiol Immunol., 342: 1-14 (2010), which is incorporated herein by reference in its entirety.

Multiple approaches to genotyping VZV have been developed. One such genotyping scheme for VZV was proposed by Faga et al. and Wagenaar et al., which amplified and sequenced 6 genes, gB (ORF 31), gE (ORF 68), gH (ORF 37), gI (ORF 67), gL (ORF 60), and IE62 (ORF 62), encompassing nearly 13 kb of the genome. Faga, B., “Review Identification and mapping of single nucleotide polymorphisms in the varicella-zoster virus genome,” Virology; 280(1):1-6 (February 2001); Wagenaar, T. R., et al., “The out of Africa model of varicella-zoster virus evolution: single nucleotide polymorphisms and private alleles distinguish Asian clades from European/North American clades,” Vaccine, 21(11-12):1072-81 (March 2003), each of which is incorporated herein by reference in its entirety. The sequenced strains were placed in a phylogenetic tree to determine relationships, with 4 major clades identified by the Faga/Wagenaar scheme. Clade A of the Faga/Wagenaar scheme was comprised primarily of European/North American isolates and includes Dumas. Clade B was an Asian (primarily Japanese) cluster and includes Oka. Clade C was an Asian-like cluster that shares some characteristics with European/North American strains, and clade D was another European/North American cluster.

Barrett-Muir et al. and Quinlivan et al. developed a different approach. Barrett-Muir, W., et al., “Investigation of varicella-zoster virus variation by heteroduplex mobility assay,” Arch Virol Suppl., (17):17-25 (2001); Barrett-Muir, W., et al., “Genetic variation of varicella-zoster virus: evidence for geographical separation of strains,” J Med Virol., 70 Suppl 1:S42-7 (2003); Quinlivan, M., et al., “The molecular epidemiology of varicella-zoster virus: evidence for geographic segregation,” J Infect Dis., 186(7):888-94 (October 2002), each of which is incorporated herein by reference in its entirety. This scheme used heteroduplex mobility assays to study VZV isolates from the United Kingdom, Africa, Asia, and Brazil to locate a number of informative SNPs across the genome (e.g., in ORFs 1, 21, 50, and 54). Using this scattered SNP scheme, strains were assigned a genotype based on shared alleles into one of four major groups. Genotype A1/A2 strains were found predominantly in Africa, Asia, and the Far East. Genotypes B and C included Dumas and were chiefly European but had also been described in North America and Brazil. Genotypes J1/J2 were Japanese isolates (including Oka) that differed from genotype A strains at 2 SNPs.

A third approach was proposed by Loparev et al. and involved sequencing a 447-bp portion of ORF 22. Loparev, V. N., et al., “Global identification of three major genotypes of varicella-zoster virus: longitudinal clustering and strategies for genotyping,” J Virol., 78(15):8349-58 (August 2004), which is incorporated herein by reference in its entirety. Based on information from a limited number of variable SNPs, Loparev et al. assigned strains one of three genotypes, either genotype E (European), genotype J (Japanese), or genotype M (mosaic). Genotype M is comprised of a mixture of E- and J-like alleles. A number of variants within genotype M had been observed, which led to further subclassification of strains as M1 or M2.

Table 17 below includes genotyping results of exemplary VZV strains based on the various approaches.

TABLE 17
Exemplary VZV Strain Genotyping

| | Accession | Faga/Wagenaar | Loparev | Barrett-Muir |
|---|---|---|---|---|
| VZV Strain | Number | Clade | Genotype | Genotype |
| Dumas | NC001348 | A | E | C |
| MSP | AY548171 | A | E | C |
| BC | AY548170 | A | E | C |
| SD | DQ479953 | A | E | C |
| KEL | DQ479954 | A | E | C |
| 36 | DQ479958 | A | E | C |
| 49 | DQ479959 | A | E | C |
| 32 P5 | DQ479961 | A | E | C |
| 32 P22 | DQ479962 | A | E | C |
| 32 P72 | DQ479963 | A | E | C |
| 11 | DQ479955 | D | E | B |
| 22 | DQ479956 | D | E | B |
| 03-500 | DQ479957 | D | E | B |
| 8 | DQ479960 | C | M2 | J1 |
| pOka | AB097933 | B | J | J1 |
| vOka | AB097932 | B | J | J2 |
| VarilRix | DQ008354 | B | J | J2 |
| VariVax | DQ008355 | B | J | J2 |

### VZV Vaccines

Varicella Vaccine: Varicella vaccines are indicated for protection against and/or prevention of primary infection by VZV (e.g., varicella). There are two varicella vaccines licensed for use in the United States. One is Varivax®, which includes live, attenuated VZV derived from the Oka VZV strain. Another varicella vaccine is ProQuad®, which also includes live, attenuated VZV derived from the Oka VZV strain, as well as vaccines against measles, mumps, and rubella.

The Oka VZV strain used in the varicella vaccine was initially obtained from a child with wild-type varicella, then introduced into human embryonic lung cell cultures, adapted to and propagated in embryonic guinea pig cell cultures and finally propagated in human diploid cell cultures. Further passage of the virus for varicella vaccine was performed in human diploid cell cultures (MRC-5) that were free of adventitious agents.

Varicella vaccine is stored as a lyophilized preparation containing sucrose, phosphate, glutamate, processed gelatin, and urea as stabilizers. Once reconstituted, each approximately 0.5-mL dose contains a minimum of 1350 plaque-forming units (PFU) of

VZV when reconstituted and stored at room temperature for a maximum of 30 minutes. Each 0.5-mL dose also contains approximately 18 mg of sucrose, 8.9 mg hydrolyzed gelatin, 3.6 mg of urea, 2.3 mg of sodium chloride, 0.36 mg of monosodium L-glutamate, 0.33 mg of sodium phosphate dibasic, 57 mcg of potassium phosphate monobasic, and 57 mcg of potassium chloride. Varivax® contains no preservative. Varivax® Prescribing Information, 2015-2020, which is incorporated herein by reference in its entirety.

Varicella vaccines are administered subcutaneously. Generally, two doses of about 0.5 ml each are administered to an individual. Administration of the two doses should be separated in time by at least three months for children who are 12 years of age and under. For individuals who are 13 years of age or older, the two doses should be administered 4 to 8 weeks apart.

Varicella vaccines are typically highly effective. Receiving a single dose of varicella vaccine is 82% effective at preventing any form of varicella and nearly 100% effective in preventing severe varicella.

In some situations, side effects observed from varicella vaccines include, but are not limited to, one or more of upper respiratory illness, cough, irritability/nervousness, fatigue, disturbed sleep, diarrhea, loss of appetite, vomiting, otitis, diaper rash/contact rash, headache, teething, malaise, abdominal pain, other rash, nausea, eye complaints, chills, lymphadenopathy, myalgia, lower respiratory illness, allergic reactions (including allergic rash, hives), stiff neck, heat rash/prickly heat, arthralgia, eczema/dry skin/dermatitis, constipation, and itching.

Zostavax®: Zostavax® is indicated for protection against secondary infection by VZV (e.g., zoster) in individuals 50 years of age or older. Zostavax® includes live, attenuated VZV. Zostavax® is a lyophilized preparation of the Oka strain of live, attenuated varicella-zoster virus (VZV).

When reconstituted, Zostavax® is a sterile suspension, and each 0.65-mL dose contains a minimum of 19,400 PFU (plaque-forming units) of Oka strain of VZV when reconstituted and stored at room temperature for up to 30 minutes. Each dose also contains 41.05 mg of sucrose, 20.53 mg of hydrolyzed porcine gelatin, 8.55 mg of urea, 5.25 mg of sodium chloride, 0.82 mg of monosodium L-glutamate, 0.75 mg of sodium phosphate dibasic, 0.13 mg of potassium phosphate monobasic, 0.13 mg of potassium chloride. Zostavax® contains no preservatives. Zostavax® Prescribing Information, 2006-2018, which is incorporated herein by reference in its entirety.

Zostavax® is administered subcutaneously. Generally, Zostavax® is administered as a single 0.65 mL dose.

The effectiveness of Zostavax® in providing protection against VZV has not been reported, but it has been reported that Zostavax® does not provide protection for all individual who receive the vaccine. The average effectiveness of Zostavax® against the development of zoster over the first 3 years following vaccination was 60% and 36% in the third year post-vaccination.

In some situations, side effects observed from Zostavax® include, but are not limited to, one or more of headache, respiratory infection, fever, flu syndrome, diarrhea, rhinitis, skin disorder, respiratory disorder, and asthenia.

Shingrix®: Shingrix® is indicated for protection against and/or prevention of secondary infection by VZV (e.g., zoster) in individuals 50 years of age or older. Shingrix® is a sterile suspension for intramuscular injection. Shingrix® includes a recombinant varicella zoster virus surface glycoprotein E (gE) antigen component. See, e.g., U.S. Pat. No. 7,939,084, which is incorporated herein by reference in its entirety. The antigen component is reconstituted at the time of use with AS01B adjuvant suspension.

The gE antigen of Shingrix® is obtained by culturing genetically engineered Chinese Hamster Ovary cells, which carry a truncated gE gene. The culture media used does not contain amino acids, albumin, antibiotics, or animal-derived proteins. Prior to lyophilized, the gE protein is purified by several chromatographic steps and formulated with excipients.

The adjuvant suspension component of Shingrix® is AS01B. AS01B is composed of 3-O-desacyl-4′-monophosphoryl lipid A (MPL) from Salmonella minnesota and QS-21, a saponin purified from plant extract Quillaja saponaria Molina, combined in a liposomal formulation. The liposomes of the formulation are composed of dioleoyl phosphatidylcholine (DOPC) and cholesterol in phosphate-buffered saline solution containing disodium phosphate anhydrous, potassium dihydrogen phosphate, sodium chloride, and water for injection. Shingrix® Prescribing Information, 2006-2018, which is incorporated herein by reference in its entirety.

After reconstitution, each 0.5-mL dose of Shingrix® is formulated to contain 50 mcg of the recombinant gE antigen, 50 mcg of MPL, and 50 mcg of QS-21. Each dose also contains 20 mg of sucrose (as stabilizer), 4.385 mg of sodium chloride, 1 mg of DOPC, 0.54 mg of potassium dihydro Treamgen phosphate, 0.25 mg of cholesterol, 0.160 mg of sodium dihydrogen phosphate dihydrate, 0.15 mg of disodium phosphate anhydrous, 0.116 mg of dipotassium phosphate, and 0.08 mg of polysorbate 80. Shingrix® does not contain preservatives.

Generally, two doses (0.5 mL each) Shingrix® are administered intramuscularly. For some individuals, the time between the two doses ranges from 2 to 6 months. For immunodeficient individuals, the time between the doses ranges from 1 to 2 months.

Shingrix® has been reported to reduce the risk of developing zoster by 97.2% in subjects aged 50 years and older.

In some situations, side effects observed from Shingrix® include, but are not limited to, one or more of pain, redness, swelling, myalgia, fatigue, headache, shivering, fever, gastrointestinal symptoms, chills, injection site pruritus, malaise, arthralgia, nausea, and dizziness. A higher risk of adverse events has been reported with the second dose of Shingrix®.

### Anti-Viral Treatments for VZV

The present disclosure provides the recognition that constructs and/or compositions described herein may be administered as part of regimen with other therapeutic agents. The present disclosure also recognizes that subjects that are administered constructs and/or compositions described herein may have previously been administered other therapeutic agents.

In some embodiments, for example, a subject may be receiving or had previously received an anti-viral agent for VZV. In some embodiments, an anti-viral agent can be administered to treat VZV infection or reactivation (e.g., varicella or zoster, respectively). In some embodiments, an anti-viral agent is or comprises acyclovir, valacyclovir, famciclovir, or a combination thereof. Table 18 below provides certain information about select anti-viral agents.

TABLE 18
Antiviral Drugs for Treating VZV

| | | | | |
|---|---|---|---|---|
| | | | | Approximate |
| | Dosage | | Most common | price for complete |
| Medication | (Pediatric) | Dosage (Adult) | adverse effects | dosing¹ |

Oral formulations:

| | | | | |
|---|---|---|---|---|
| acyclovir | ≥2 years old; 20 | 800 mg 5×/day; 7- | malaise, headache, | 35-800 mg tabs; $70 |
| | mg/kg 4×/day; | 10 days² | nausea, vomiting | |
| | max. 3.2 g/day; 5 | | | |
| | days² | | | |
| valacyclovir | 2-18 years old; 20 | 1 g 3×/day; 7 days² | headache, nausea, | 21-1 g tabs; $220 |
| | mg/kg 3×/day; | | abdominal pain | |
| | max. 1 g 3×/day; 5 | | | |
| | days² | | | |
| famciclovir | not established | 500 mg 3×/day³; 7 | headache, nausea, | 21-500 mg tabs; |
| | | days² | fatigue, diarrhea | $184 |

Intravenous formulations:

| | | | |
|---|---|---|---|
| acyclovir | 250 mg/m2 every | 10-15 mg/kg every | phlebitis, acute renal |
| | 8 h; 5-7 days² | 8 h; 5-7 days² | failure, nausea, |
| | | | vomiting, rash |
| foscarnet⁴ | not established | 180 mg/kg/day in 2- | fever, headache, |
| | | 3 divided doses; 7- | renal dysfunction, |
| | | 14 days² | electrolyte |
| | | | abnormalities, |
| | | | nausea, vomiting |
| | | | diarrhea, anemia, |
| | | | granulocytopenia |

### Cytomegalovirus (CMV)

Cytomegalovirus (CMV) is a genus of Herpesvirus in the order Herpesvirales, in the family Herpesviridae, and in the subfamily Betaherpesvirinae. There are nine distinct human herpesvirus (HHV) species known to cause human diseases such as HHV-1, HHV-2, HHV-3, HHV-4, HHV-5, HHV-6A, HHV-6B, HHV-7, HHV-8. HHV-5 is also known as human cytomegalovirus (CMV).

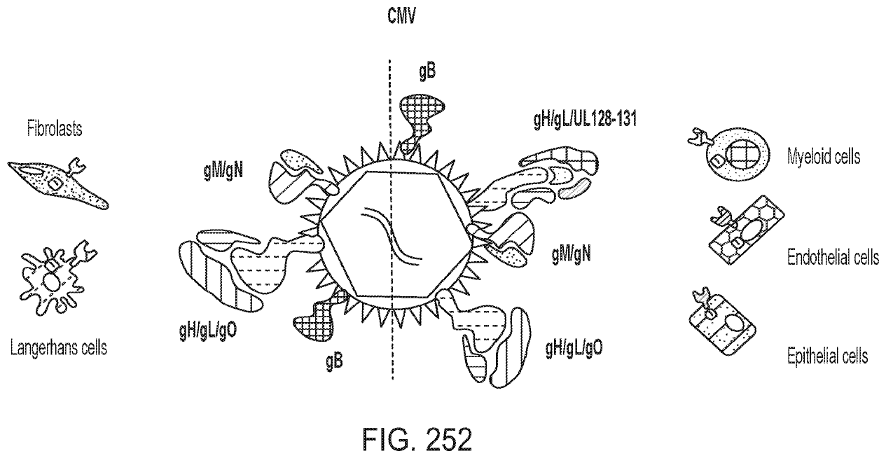

As shown in FIG. 252, CMV structure mainly consist of DNA core, capsid, tegument and envelope from inside to outside. CMV has a double-stranded DNA genome of about 230 kb, which is complexed helically to form a DNA core. The DNA core is enclosed in a capsid composed of a total of 162 capsomere protein subunits. CMV's capsid has a diameter of about 100 nm and is surrounded by a tegument, which is enclosed by a lipid bilayer envelope containing viral glycoproteins to give a final diameter of about 180 nm for mature infectious viral particles (virions). Griffiths P. D., et al., “Molecular biology and immunology of cytomegalovirus,” Biochem. J., 241:313-324 (1987), which is incorporated herein by reference in its entirety.

The tegument contains most of CMV's proteins. The lower matrix phosphoprotein 65 (pp65) (also referred as unique long 83 (UL83)) is the most abundant CMV protein. Tegument proteins of CMV play an important role for the assembly of virions during proliferation and the disassembly of the virions during entry. CMV tegument proteins also modulate the host cell responses for viral infection. Crough T., et al., “Immunobiology of human cytomegalovirus: From bench to bedside,” Clin. Microbiol. Rev. 76-98 (2009), which is incorporated herein by reference in its entirety. The viral envelope surrounding the tegument contains more than 20 glycoproteins that are involved in the attachment and penetration of host cells. The envelope glycoproteins include glycoprotein B, H, L, M, N, and 0.

CMV infection can cause a broad range of diseases, including pneumonia, retinitis, gastrointestinal diseases, mental retardation and vascular disorders. CMV is also known to be a major cause of morbidity and mortality for humans. Dunn W., et al., “Functional profiling of a human cytomegalovirus genome,” Proc. Natl. Acad. Sci. USA., 100:14223-14228 (2003); Scholz M., et al., “Inhibition of cytomegalovirus immediate early gene expression: A therapeutic option?” Antivir. Res., 49:129-145 (2001); Marschall M., et al., “Recombinant green fluorescent protein-expressing human cytomegalovirus as a tool for screening antiviral agents. Antimicrob,” Agents Chemother., 44:1588-1597 (2000), each of which is incorporated herein by reference in its entirety. Furthermore, congenital infection is a major problem with CMV in that it can result in a severe cytomegalic inclusion disease of the neonate, mucoepidermoid carcinoma, and other malignancies. Marschall (2000); Melnick M., et al., “Human cytomegalovirus and mucoepidermoid carcinoma of salivary glands: Cell-specific localization of active viral and oncogenic signaling proteins is confirmatory of a causal relationship,” Exp. Mol. Pathol., 92:118-125 (2012), which is incorporated herein by reference in its entirety.

Typically, CMV causes moderate or subclinical diseases in healthy adults. In fact, most CMV infections are silent and CMV rarely causes signs or symptoms in healthy people. However, CMV can be life-threatening for immunocompromised, immunosuppressed and immunonaive patients, for example, newborn infants, elderly individuals, sick individuals, acquired immunodeficiency syndromes (AIDS) patients, and organ transplant recipients. Individuals generally considered at higher risk of CMV infections encompass newborns infected through their mothers before birth, babies infected through breast milk and people with weakened immune systems such as organ transplantation recipients or immunodeficient patients.

CMV is transmitted by close interpersonal contact such as saliva, semen, urine, breast milk, or vertically transmission which viruses pass the placenta and directly infect the fetus. Fowler K. B., et al., “Maternal immunity and prevention of congenital cytomegalovirus infection,” JAMA, 289:1008-1011 (2003); Yamamoto A. Y., et al., “Human cytomegalovirus reinfection is associated with intrauterine transmission in a highly cytomegalovirus-immune maternal population,” Am. J. Obstet. Gynecol., 202:297-e1 (2010), each of which is incorporated herein by reference in its entirety. CMV is the leading cause of congenital viral infection. Dollard S. C., et al., “New estimates of the prevalence of neurological and sensory sequelae and mortality associated with congenital cytomegalovirus infection,” Rev. Med. Virol., 17:355-363 (2007); Kenneson A., et al., “Review and meta-analysis of the epidemiology of congenital cytomegalovirus (CMV) infection,” Rev. Med. Virol., 17:253-276 (2007); Nyholm J. L., et al., “Prevention of maternal cytomegalovirus infection: Current status and future prospects,” it. J. Women's Health, 2:23 (2010); Leung A. K., et al., “Congenital cytomegalovirus infection,” J. Natl. Med. Assoc., 95:213 (2003), each of which is incorporated herein by reference in its entirety. As mentioned above, CMV infection is mostly or mildly asymptomatic among the general population (85%-90%). However, around 10%1-15% of infants with the congenital infection may be at risk of sequelae such as mental retardation, jaundice, hepatosplenomegaly, microcephaly, hearing impairment and thrombocytopenia. Dreher A. M., et al., “Spectrum of disease and outcome in children with symptomatic congenital cytomegalovirus infection,” J. Pediatrics, 164:855-859 (2014); Swanson E. C., et al., “Congenital cytomegalovirus infection: New prospects for prevention and therapy,” Pediatric Clin., 60:335-349 (2013); Yamamoto A. Y., et al., “Congenital cytomegalovirus infection as a cause of sensorineural hearing loss in a highly immune population,” Pediatric Infect. Dis. J., 30:1043 (2011); Fowler K. B., et al., “Congenital cytomegalovirus (CMV) infection and hearing deficit,” J. Clin. Virol., 35:226-231 (2006), each of which is incorporated herein by reference in its entirety. Among these risks, some of the most challenging are those that impact the central nervous system (CNS) sequelae related to neurodevelopment, including mental retardation, seizures, hearing loss, ocular abnormalities and cognitive impairment. Boppana S. B., et al., “Congenital cytomegalovirus infection: Clinical outcome,” Clin. Infect. Dis., 57:S178-S181 (2013); Mussi-Pinhata M. M., et al., “Birth prevalence and natural history of congenital cytomegalovirus infection in a highly seroimmune population,” Clin. Infect. Dis., 49:522-528 (2009); Pass R. F., et al., “Congenital cytomegalovirus infection following first trimester maternal infection: Symptoms at birth and outcome,” J. Clin. Virol., 35:216-220 (2006), each of which is incorporated herein by reference in its entirety. That means the asymptomatic newborns with CMV infection still have an increased risk for long-term sequelaes, especially, mental retardation and sensorineural hearing loss (SNHL) (Nassetta L., et al., “Treatment of congenital cytomegalovirus infection: Implications for future therapeutic strategies,” J. Antimicrob. Chemother. 2009; 63:862-867 (2009); Barbi M., et al., “Multicity Italian study of congenital cytomegalovirus infection,” Pediatric Infect. Dis. J., 25:156-159 (2006); Coll O., et al., “Guidelines on CMV congenital infection,” J. Perinat. Med., 37:433-445 (2009); Schleiss M. R., “Congenital cytomegalovirus infection: Update on management strategies,” Curr. Treat. Options Neurol., 10:186-192 (2008), each of which is incorporated herein by reference in its entirety), making CMV the leading nonhereditary cause of SNHIL (Fowler (2006); Nance W. E., et al., “Importance of congenital cytomegalovirus infections as a cause for pre-lingual hearing loss,” J. Clin. Virol., 35:221-225 (2006), each of which is incorporated herein by reference in its entirety).

### Lifecycle

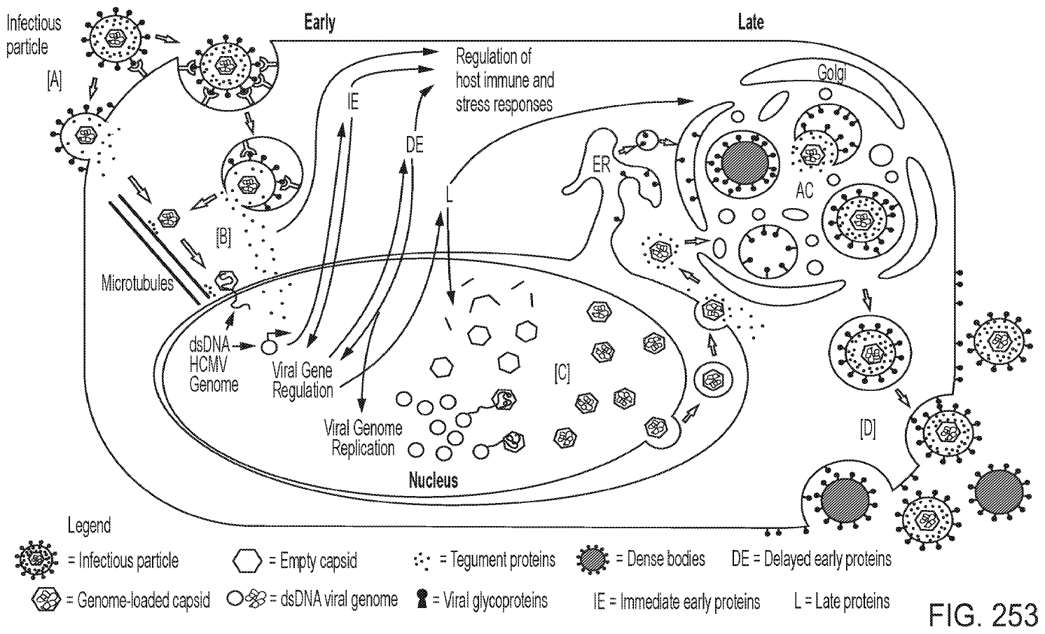

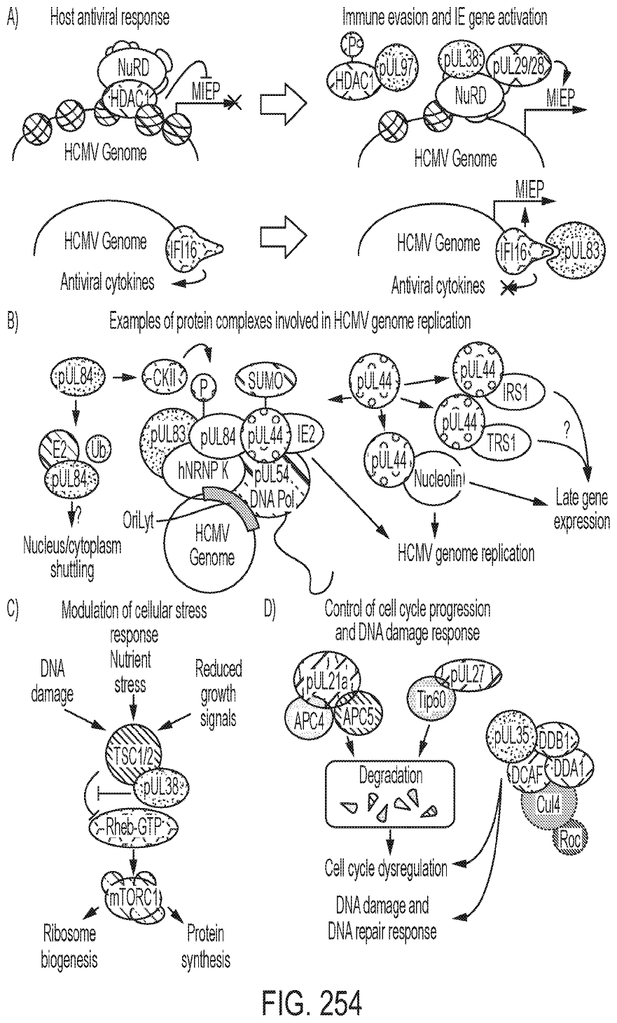

As shown in FIG. 253, CMV infection begins when a virion attaches to a host cell with specific receptors on the cellular surface. Capsid and tegument proteins are delivered to the cell's cytosol. The CMV capsid travels to the nucleus, where the genome is delivered and circularized. Tegument proteins regulate host cell responses and initiate the temporal cascade of the expression of viral I immediate early (IE) genes, followed by delayed early (DE) genes, which initiate viral genome replication, and late (L) genes. Late gene expression initiates capsid assembly in the nucleus, followed by nuclear egress to the cytosol. Capsids associate with tegument proteins in the cytosol and are trafficked to the viral assembly complex (AC) that contains components of the endoplasmic reticulum (ER), Golgi apparatus and endosomal machinery. The CMV further acquire tegument and viral envelope by budding into intracellular vesicles at the AC. Enveloped infectious CV particles are released along with non-infectious dense bodies.

Alternatively, some viral genes may transcribe latency associated transcripts to accumulate in host cells. As such, CMV can persist in host cells indefinitely to have a latent infection pathway. While primary infection may be accompanied by limited illness, long-term latency is often asymptomatic. Instead, the viruses are persistent in the host, not causing any adverse reactions, but can be transmitted to other hosts by direct contact. When CMV are stimulated by explanation or their host immune system is suppressed, the dormant viruses can reactivate to begin generating large number of viral progenies to cause symptoms and diseases, described as the lytic life cycle. Chen Y.-C., et al., “Potential application of the CRISPR/Cas9 system against herpesvirus infections,” Viruses, 10:291 (2018); Porter K. R., et al., “Reactivation of latent murine cytomegalovirus from kidney,” Kidney Int., 28:922-925 (1985), each of which is incorporated herein by reference in its entirety.

CMV latency has been characterized as episomal latency, which is essentially quiescent in myeloid progenitor cells. CMV can be reactivated by differentiation, inflammation, immunosuppression or critical diseases. Dupont L., et al., “Cytomegalovirus latency and reactivation: Recent insights into an old age problem,” Rev. Med. Virol., 26:75-89 (2016), which is incorporated herein by reference in its entirety. Latency is a specific phase in CMV life cycles in which virions stop producing posterior to infection, but the viral genome has not been entirely removed from host cells. In some cases, reactivation of latent CMV infections can lead to health risk.

Generally, the primary target cells of CMV are monocytes, lymphocytes, and epithelial cells. The major sites of CMV latency are peripheral monocytes and CD34+ progenitor cells. After infection, CMV is recurrent and competent to remain latent within the body over long periods. Dunn W., et al., “Functional profiling of a human cytomegalovirus genome,” Proc. Natl. Acad. Sci. USA., 100:14223-14228 (2003); Scholz M., et al., “Inhibition of cytomegalovirus immediate early gene expression: A therapeutic option?” Antivir. Res., 49:129-145 (2001), each of which is incorporated herein by reference in its entirety. In all patients, reactivation of latent HCMV can damage tissues and lead to organ disease, and reactivated CMV may trigger indirect immunomodulatory effects to cause detrimental outcomes, including increased mortality and graft rejection of organ transplantation in recipients. La Y., et al., “Human cytomegalovirus seroprevalence and titers in solid organ transplant recipients and transplant donors in Seoul, South Korea,” BMC Infect. Dis., 19:948 (2019), which is incorporated herein by reference in its entirety.

### CMV Genome

CMV has the largest genome of any known human virus, which is approximately 236 kilobases in size. A CMV genome is a linear, double-stranded DNA molecule comprising two unique regions, each flanked by inverted repeats. The structure can be represented by the formula ab-U_{L}-b′a′c′—U_{S}-ca, where U_{L} and U_{S} denote the long and short unique regions and ba/b′a′ and ca/c′a′ indicate the inverted repeats.

Studies have shown that CMV strains are divergent in a subset of genes encoding membrane-associated or secreted proteins. These genes are referred to as “hypervariable genes.” Each of these hypervariable genes exists as several highly divergent clusters of alleles, with a much lower level of allelic variation evident within individual clusters. Sequences of particular alleles have been reported to be stable on short timescales in patients and during cell culture. These observations suggest that hypervariation is a result of immune selection and that the allelic clusters have a long history, perhaps having emerged during the evolution of populations of early humans or their predecessors. Evidence also exists demonstrating that recombination has occurred during CMV evolution, and that individual CMV infections can involve multiple strains. These variations add complexities to assessments of the associations between the genetic constitution of CMV strains and disease outcome. They can also complicate development of vaccines. The present disclosure recognizes this variation and proposes particular antigens that can be useful in pharmaceutical compositions (e.g., immunogenic compositions, e.g., vaccines) that provide protection against a wide variety of CMV strains and variants.

### CMV Vaccines

As early as the 1970s, CMV vaccines based on live attenuated viruses were developed. Plotkin, S., et al., “Towne-vaccine-induced prevention of cytomegalovirus disease after renal transplants,” Lancet, 323, 528-530 (1984), which is incorporated herein by reference in its entirety. However, immune responses were lower than hoped, and the incidence and severity of infection were only partially reduced. Plotkin (1984), which is incorporated herein by reference in its entirety. One of these vaccines contained the Towne strain of CMV. While it was found to be safe and well tolerated, it was not entirely effective. For example, when it was administered to kidney transplant recipients, the vaccine was effective in reducing the clinical manifestation of CMV disease and the risk of graft rejection (Plotkin (1984), which is incorporated herein by reference in its entirety), but was not able to reduce the risk of CMV infection. The Towne strain vaccine also failed to prevent CMV infection in healthy women exposed to infected children in day care. Plotkin, S. A., “Protective effects of Towne cytomegalovirus vaccine against low-passage cytomegalovirus administered as a challenge,” J. Infect. Dis., 159, 860-865 (1989), which is incorporated herein by reference in its entirety. Finally, the vaccine provided protection from infection in healthy volunteers exposed to low doses of unattenuated CMV but was less effective than previously acquired natural immunity when higher doses were used. Plotkin (1989), which is incorporated herein by reference in its entirety. These results led to the conclusion that the Towne virus might be over-attenuated and therefore incapable of conferring sufficient immune responses as a vaccine. Prichard, M. N., et al., “A review of genetic differences between limited and extensively passaged human cytomegalovirus strains,” Rev. Med. Virol., 11, 191-200 (2001), which is incorporated herein by reference in its entirety.

To improve immunogenicity, recombination of the Towne strain with a non-attenuated Toledo strain was performed, which substituted some regions from the Toledo virus genome with the corresponding regions of the Towne virus genome. Heineman, T., et al., “Preliminary safety results from a phase 1 study of four new live, recombinant HCMV Towne/Toledo chimeric vaccines,” In Proceedings of the Abstracts of the 27th Herpes Simplex Virus Workshop, Cairns, Australia, 20-26 Jul. 2002, which is incorporated herein by reference in its entirety. Four Towne/Toledo chimaera vaccines were prepared and tested in seronegative men receiving different subcutaneous doses of the new viruses. All the studied preparations were found to be safe and well tolerated. However, despite differences among preparations, recombinant CMV strains were less successful than wild-type CMV in enhancing vaccine-elicited immune responses. Adler, S. P., et al., “A Phase 1 Study of 4 Live, Recombinant Human Cytomegalovirus Towne/Toledo Chimera Vaccines in Cytomegalovirus-Seronegative Men,” J. Infect. Dis., 214, 1341-1348 (2016), which is incorporated herein by reference in its entirety.

More recently, additional attempts to develop a live attenuated CMV vaccine have been made. To improve immunogenicity and efficacy, a CMV virus was modified to restore the expression of genes that have been found to be of relevance in the development of protective immunity. Adler (2016), which is incorporated herein by reference in its entirety. For example, a preparation containing a replication-defective CMV virus with restored expression of the gH/gL/pUL128-131 pentameric complex was evaluated for phase I vaccine safety and immunogenicity in CMV-seronegative and CMV-seropositive adults. In seronegative subjects, a significant increase in neutralizing antibody concentrations was evidenced with levels that, one month after the third dose, were comparable with those detected after the natural infection for both neutralizing antibody levels and the cellular response. Adler (2016), which is incorporated herein by reference in its entirety. In particular, antibodies capable of neutralizing epithelial cell infection were detected.

Additional CMV vaccine development has occurred around making vaccines including CMV-neutralizing proteins evoked by natural infection. Some vaccines incorporated glycoprotein B (gB) in association with an adjuvant. For instance, a vaccine comprising gB with MF59, an oil-in-water adjuvant (O'Hagan, D. T., et al., “The history of MF59® adjuvant: A phoenix that arose from the ashes,” Expert Rev. Vaccines, 12, 13-30.37 (2013), which is incorporated herein by reference in its entirety) was shown to be safe, immunogenic, and capable of providing 50% protection against primary infection in seronegative postpartum women (Pass, R. F., et al., “Vaccine prevention of maternal cytomegalovirus infection,” N. Engl. J. Med., 360, 1191-1199 (2009), which is incorporated herein by reference in its entirety) and 43% protection in seronegative adolescent girls (Bernstein, D. I., et al., “Safety and efficacy of a cytomegalovirus glycoprotein B (gB) vaccine in adolescent girls: A randomized clinical trial,” Vaccine, 34, 313-319.39 (2016), which is incorporated herein by reference in its entirety). Administration in transplant patients limited the periods of viremia and the need for antiviral treatment. Rieder, F., et al., “Cytomegalovirus vaccine: Phase II clinical trial results,” Clin. Microbiol. Infect., 20 (Suppl. 5), 95-102 (2014), which is incorporated herein by reference in its entirety. Long-term efficacy of gB-only vaccines is questionable, however, as the serum antibody levels and protection waned with time. Administration of further doses as a booster did improve protection. Sabbaj, S., et al., “Glycoprotein B Vaccine Is Capable of Boosting Both Antibody and CD4 T-Cell Responses to Cytomegalovirus in Chronically Infected Women,” J. Infect. Dis., 203, 1534-1541 (2011), which is incorporated herein by reference in its entirety. Better immunogenicity was found when gB was combined with AS01, an adjuvant containing the immunostimulants 3-O-desacyl-40-monophosphoryl lipid A and QS-21, which promote Toll-like receptor 4 activity. Gareon, N., et al., “Recent clinical experience with vaccines using MPL- and QS-21-containing adjuvant systems,” Expert Rev. Vaccines, 10, 471-486 (2011), which is incorporated herein by reference in its entirety. Higher and more persistent antibody titers were detected from gB vaccines including AS01. Cui, X., et al., “Novel trimeric human cytomegalovirus glycoprotein B elicits a high-titer neutralizing antibody response,” Vaccine, 36, 5580-5590 (2018), which is incorporated herein by reference in its entirety. Efficacy trials have not yet been performed with this type of CMV vaccine.

Further studies have shown that administration of a pentameric complex of proteins present on the surface of CMV could induce a greater production of neutralizing antibodies than gB. Wang, D., et al., “Human cytomegalovirus virion protein complex required for epithelial and endothelial cell tropism,” Proc. Natl. Acad. Sci. USA, 102, 18153-18158 (2005), which is incorporated herein by reference in its entirety. The pentameric complex, comprising glycoprotein H (gH), glycoprotein L (gL), and UL128, UL130, and UL131 gene products, was found to generate the most effective immune response against CMV endothelial and epithelial cell entry. Wang (2005), which is incorporated herein by reference in its entirety. Moreover, in seronegative pregnant women exposed to the virus, it was evidenced that the immune response to this pentameric complex offered the greatest activity against transmission of CMV to the fetus. Lilleri, D., et al., “Maternal immune correlates of protection from human cytomegalovirus transmission to the fetus after primary infection in pregnancy,” Rev. Med. Virol., 27, e1921 (2017), which is incorporated herein by reference in its entirety.

Potential efficacy for the prevention of cCMV infection was shown by some vaccines that utilize viral vectors to obtain an immune response against some viral components. One of these vaccines, a vaccine based on a canarypox virus expressing gB (ALVACCMV), was found to be effective when used as a prime for live-attenuated strains. Adler, S. P., et al., “A canarypox vector expressing cytomegalovirus (CMV) glycoprotein B primes for antibody responses to a live attenuated CMV vaccine (Towne),” J. Infect Dis., 180, 843-846 (1999), which is incorporated herein by reference in its entirety. In subjects receiving the vectored gB vaccine and later immunized with the Towne vaccine, neutralizing antibody titers against gB developed sooner, were much higher in number, and persisted longer than in subjects receiving only gB. Similar canarypox vaccines expressing CMV pp65 induced long-lasting cytotoxic T lymphocyte (CTL) responses in all originally seronegative volunteers, with a CTL precursor frequency similar to that of naturally seropositive individuals. Adler (1999), which is incorporated herein by reference in its entirety.

The addition of proteins that evoke neutralizing antibodies, and proteins that strongly stimulate cell-mediated immunity, has presented a promising option for limiting fetal infection and CMV disease in transplanted patients. In a phase II study, a bivalent CMV-DNA vaccine encoding both gB and pp65A was found to elicit robust pp65-specific T cell responses and late gB-specific B cell responses. A phase III trial assessing its efficacy in the prevention of mortality in seropositive transplant recipients has been initiated. U.S. National Library of Medicine, ClinicalTrials gov., “A Study to Evaluate a Therapeutic Vaccine, ASP0113, in Cytomegalovirus (CMV)-Seropositive Recipients Undergoing Allogeneic, Hematopoietic Cell Transplant (HCT) (HELIOS), which is incorporated herein by reference in its entirety.

Additionally, a vaccine based on the modified vaccinia Ankara (MVA) that encodes three immunodominant CMV antigens (pp65, IE1-exon4, and IE2-exon5) was found to be able to safely and durably expand high levels of CMV-specific T cells when tested in a phase 1 trial in healthy adults. La Rosa, C., et al., “MVA vaccine encoding CMV antigens safely induces durable expansion of CMV-specific T cells in healthy adults,” Blood, 129, 114-125 (2017), which is incorporated herein by reference in its entirety. In a phase II randomized clinical trial, the administration of this vaccine led to a reduction in CMV viremia risk in transplant patients. Reactivation of CMV occurred in 9.8% of vaccine recipients compared to 19.6% of patients given a placebo (hazard ratio, 0.46 [95% CI, 0.16 to 1.4]; p=0.075). Levels of long-lasting, pp65-specific T cells with the effector memory phenotype were significantly higher in subjects with the vaccine than in the placebo group. Aldoss, I., et al., “TRIPLEX VACCINE Study Group: Poxvirus Vectored Cytomegalovirus Vaccine to Prevent Cytomegalovirus Viremia in Transplant Recipients: A Phase 2, Randomized Clinical Trial,” Ann. Intern. Med., 172, 306-316 (2020), which is incorporated herein by reference in its entirety.

Several peptide, DNA, and mRNA vaccines coding for pp65, gB, or other relevant CMV proteins have also been studied to improve immunogenicity and efficacy. La Rosa, C., et al., “Clinical evaluation of safety and immunogenicity of PADRE-cytomegalovirus (CMV) and tetanus-CMV fusion peptide vaccines with or without PF03512676 adjuvant,” J. Infect. Dis., 205, 1294-1304 (2012); Wloch, M. K., et al., “Safety and immunogenicity of a bivalent cytomegalovirus DNA vaccine in healthy adult subjects,” J. Infect. Dis., 197, 1634-1642 (2008); Geall, A. J., et al., “Nonviral delivery of self-amplifying RNA vaccines,” Proc. Natl. Acad. Sci. USA, 109, 14604-14609 (2012); Smith, L. R., et al., “Clinical Development of a Cytomegalovirus DNA Vaccine: From Product Concept to Pivotal Phase 3 Trial,” Vaccines, 1, 398-414 (2013); Perotti, M., et al., “Rationally designed Human Cytomegalovirus gB nanoparticle vaccine with improved immunogenicity,” PLoS Pathog, 16, e1009169 (2016); Contreras, H., et al., “MVA-vectored pentameric complex (PC) and gB vaccines improve pregnancy outcome after guinea pig CMV challenge, but only gB vaccine reduces vertical transmission,” Vaccines, 7, 182 (2019); John, S., et al., “Multi-antigenic human cytomegalovirus mRNA vaccines that elicit potent humoral and cell-mediated immunity,” Vaccine, 36, 1689-1699 (2018), each of which is incorporated herein by reference in its entirety. Some of them have shown preliminary evidence of efficacy in transplant recipients. For example, a CMV gB nucleoside-modified mRNA vaccine elicited an antibody response with greater durability and breadth than in the MF59-adjuvanted gB protein immunization. Nelson, C. S., et al., “Human Cytomegalovirus Glycoprotein B Nucleoside-Modified mRNA Vaccine Elicits Antibody Responses with Greater Durability and Breadth than MF59-Adjuvanted gB Protein Immunization,” J. Virol, 94, e00186-20 (2020), which is incorporated herein by reference in its entirety.

One mRNA vaccine is the mRNA-1647 vaccine. It comprises five mRNAs encoding the subunits of the pentamer complex and one mRNA encoding the gB target antigen. Phase I (NCT03382405) and phase II (NCT04232280) studies specifically devoted to evaluating the immunogenicity, safety and tolerability, and the most effective dosage in humans have been carried out and partly completed. Interim analysis of phase II trial results reported the substantial ability of the vaccine to induce an immune response significantly greater than that evoked by the natural infection of both seronegative and seropositive individuals.

In addition to the above approach, virus-like particles and nanoparticles have been developed for multivalent antigen presentation. A virus-like particle with gB on the surface has shown a high induction of neutralizing antibodies in animals. Moreover, pp65-derived peptides combined with a tetanus toxin epitope have exhibited immunogenicity in humans. Perotti, M., et al., “Virus-Like Particles and Nanoparticles for Vaccine Development against HCMV,” Viruses, 12, 35 (2019), which is incorporated herein by reference in its entirety.

Table 19 below summarizes certain of the CMV vaccines discussed herein. Esposito, S., et al., “Prevention of Congenital Cytomegalovirus Infection with Vaccines: State of the Art,” Vaccines, 9:523 (2021), which is incorporated herein by reference in its entirety.

TABLE 19
Certain CMV Vaccines Under Clinical Development

| Category | Vaccine | Antigen(s) | Adjuvant | Manufacturer | Clinical Trial | Phase |
|---|---|---|---|---|---|---|
| Attenuated | V160-001 | gB, pp65, | | MSD, CMV | NCT019869010 | 1 |
| and Disabled | Towne-Toledo | IE1 | | Research | | |
| Infectious | Chimera | | | Foundation, | | |
| Single Cycle | Vaccine | | | International | NCT01195571 | 1 |
| (DISC) | | | | AIDS Vaccine | | |
| Vaccines | | | | Institute | | |
| | VCL-CT02 Plu | gB, pp65, | | UC-SF, Vical | NCT00370006 | 1 |
| | Towne CMV | IE1 | | | NCT00373412 | 1 |
| Recombinant | GSK1492903A | gB | | GlaxoSmithKline | NCT00435396 | 1 |
| Subunit | | | | | NCT01357915 | 1 |
| Vaccines | gB Subunit | gB | | University | NCT00299260 | 2 |
| | | | | College, London | NCT01883206 | 2 |
| | gB/MF59 | gB | | NIAID | NCT00133497 | 2 |
| | | | | | NCT00815165 | 2 |
| | gB/MF59 | gB | | Sanofi Pasteur | NCT00125502 | 2 |
| Vectored | AVX601 | gB, pp65, | | Alpha Vax, Inc. | NCT00439803 | 1 |
| Vaccines | HCMV-MVA | IE1 | | (Novartis) | NCT01941056 | 1 |
| | Triplex | | | | NCT02506933 | 2 |
| | HB-101 | pp65, IE1- | | City of Hope, | NCT02798692 | |
| | ALVAC-pp65 | exon4, IE2- | | National Cancer | NCT00353977 | |
| | | exon5, gB, | | Institute Hookipa | | |
| | | pp65, pp65 | | Biotech NHLBI | | |
| Nucleic Acid | ASP0113 | gB, pp65 | | Astellas, Vical | NCT02103426 | 1 |
| Vaccines | | | | | NCT01903928 | 2 |
| | | | | | NCT01974206 | 2 |
| | VCL-CB01 | gB, pp65 | | Astellas, Vical | NCT01877655 | 3 |
| | | | | | NCT00285259 | 2 |
| Peptide | CMVpp65- | gB, pentamer | | Moderna | NCT03382405 | 1 |
| Vaccines | A*0201 peptide | complex; | | | NCT04232280 | 2 |
| | containing | pp65; | | | | |
| | helper T | T cell epitope | | | | |
| | lymphocyte | fused to | | | | |
| | | either | | | | |
| | | PADRE or | | | | |
| | | CMV | | | | |
| | | tetanus | | | | |
| | | epitope | | | | |
| | PADRE peptide | pp65; T cell | | City of Hope, | NCT00712634 | 1 |
| | or tenanus | epitope fused | | National | NCT00722839 | 1 |
| | toxoid peptide | to | | Cancer Institute | NCT01588015 | 1 |
| | Tetanus-HCMV | tetanus | | City of Hope, | NCT02396134 | 2 |
| | pp65 fusion | epitope | | National | | |
| | peptide | | | Cancer Institute | | |

### Anti-Viral Treatments for CMV

The present disclosure provides the recognition that constructs and/or compositions described herein may be administered as part of regimen with other therapeutic agents. The present disclosure also recognizes that subjects that are administered constructs and/or compositions described herein may have previously been administered other therapeutic agents.

In some embodiments, for example, a subject may be receiving or had previously received an anti-viral agent for CMV. In some embodiments, an anti-viral agent can be administered to treat CMV infection. In some embodiments, an anti-viral agent is or comprises maribavir, ganciclovir, ganciclovir, valganciclovir, foscarnet, cidofovir, letermovir, or a combination thereof.

### Norovirus

Noroviruses are members of the Caliciviridae family of small, non-enveloped, positive-stranded RNA viruses. The Norovirus genus includes both human and animal (e.g., murine and canine) noroviruses.

Noroviruses typically have a 24-48 hour incubation period between infection and development of symptoms. Symptoms typically persist for 12-72 hours, but reports have indicated that viral shedding can continue long after symptoms have resolved. It is believed that viral shedding can continue for several days or even 1-2 weeks after symptoms have resolved; immunocompromised individuals may continue shedding virus even longer, up to several (e.g., 3, 4, 5, 6, 7, 8 or more) months after infection.

Noroviruses are highly infectious; it has been reported that doses as low as 20 viral particles may be sufficient to establish infection. Exposure is typically via inhalation or ingestion (e.g., commonly by oral exposure, such as by ingestion of contaminated food). Norovirus virions withstand acidic pH and can survive passage through the stomach.

Given the above-noted low infection dose and long shedding periods, combined with the high levels of shedded virus (10⁸-10¹⁰ copies of RNA per g) often detected in feces, norovirus infections can spread rapidly within communities.

Norovirus infection can be asymptomatic, particularly in children (see, for example, Robilotti et al., Clin. Microbiol. Rev., 28:134, 2015 and references cited therein). Symptomatic infection typically results in acute gastroenteritis, characterized by symptoms such as vomiting and diarrhea, and/or nausea and severe abdominal cramps. Other reported associated conditions include encephalopathy, intravascular coagulation, necrotizing enterocolitis in premature infants, postinfectious irritable bowel syndrome, and benign infantile seizures. Young children, the elderly, and immunocompromised individuals (e.g., transplant patients or other subjects receiving immunosuppressive medication or therapy) are among those most susceptible to development of serious disease.

Although it has been reported that 20-30% of cases of norovirus infection in humans can be asymptomatic or “mild” (e.g., resolving within a few days) (Qi et al. Am J Infect Control. 43:833, 2015, doi: 10.1016/j.ajic.2015.04.182; Marshall et al., J Med Virol. 69:568, 2003, doi: 10.1002/jmv.10346), norovirus infection remains a significant risk. Mortality may be as high as 3%, and norovirus infections are believed to be responsible for up to 20% of emergency room visits and hospitalizations, even in middle- to high-income countries (Lopman et al., PLoSMed. 13:e1001999, 2016, doi: 10.1371/journal.pmed.1001999). Dehydration associated with norovirus infection can be particularly problematic, particularly in the elderly and/or the very young. Furthermore, in some instances (e.g., in immunocompromised subjects including, for example, transplant patients, patients receiving chemotherapy or immunosuppressive therapy, subjects infected with HIV, etc.) (Cardemil et al. Infect Dis Clin North Am. 31:839, 2017, doi: 10.1016/j.idc.2017.07.012), norovirus infection can become chronic, with serious consequences (Bok et al., Oncol Nurs Forum. 40:434, 2013, doi: 10.1188/13.ONF.434-436; Kaufman et al. Antiviral Res. 105C:80, 2014, doi: 10.1016/j.antiviral.2014.02.012; Trivedi et al. Am J Infect Control. 41:654, 2013, doi: 10.1016/j.ajic.2012.08.002). Without wishing to be bound by any particular theory, the present disclosure proposes that a robust T cell immunization, e.g., as may be achieved as described herein (e.g., via administration or delivery of one or more T cell epitopes as described here, for example via string constructs), may be particularly useful or effective to protect against chronic infection, e.g., by facilitating removal of infected cells.

No commercial vaccines or specific antivirals are currently available to treat or prevent human norovirus infections. Standard of care remains supportive therapy, particularly to address dehydration and/or electrolyte abnormalities. Some reports have suggested that administration of nitazoxanide may be helpful, for example, to reduce the duration of illness (see, for example, Rossignol et al. Aliment Pharmacol Ther 24:1423, 2006, doi.org/10.1111/j.1365-2036.2006.03128.x.). Enteric administration of human immunoglobulin has also been reported to assist in resolution of diarrhea associated with norovirus infection (see, for example, Chagla et al, J Clin Virol 58:306, 2013, doi.org/10. 1016/j.jcv.2013.06.009).

Furthermore no small animal models have been described that mimic human disease; only recently has an in vivo model (in zebrafish larvae) been shown to support norovirus replication (e.g., of GII.3 and GII.4 variants; see Van Dycke et al., PLoS Pathog. 15:e1008009, 2019, doi: 10.1371/journal.ppat.1008009). Some in vitro replication models have been described; specifically, some strains (e.g., Gii.4-Sydney) have been shown to replicate in human B cells (see, for example, Lindesmith et al., J Infect Dis. 216:1227, 2017, doi: 10.1093/infdis/jix385); and some (e.g., some GII.3 and some GII.4 strains) have been shown to replicate in human intestinal enteroid monolayer cultures (see, for example, Ettayebi et al., Science. 353:1387, 2016, doi: 10.1126/science.aaf5211). Also, a monoclonal antibody (NV8812; see White et al. J Virol. 70:6589-97. doi: 10.1128/JVI.70.10.6589, 1996) to the viral VP1 protein that has been reported to bind to the C-terminal region at residues 300-384, has been reported to block binding of virus-like particles (VLPs) comprising the norovirus VP1 protein to relevant human and animal cells.

An effective norovirus vaccine remains an unmet medical need of critical importance for global health.

### Lifecycle

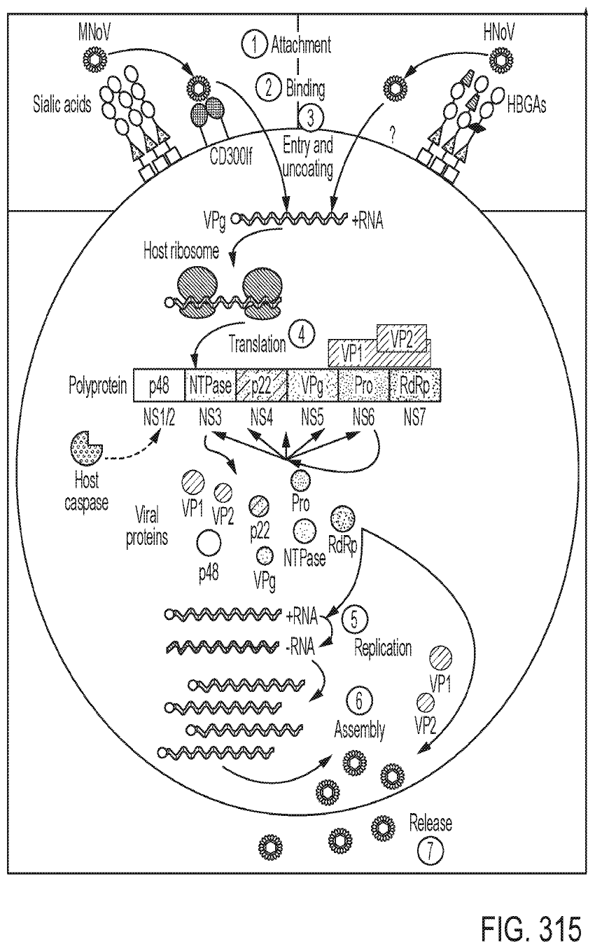

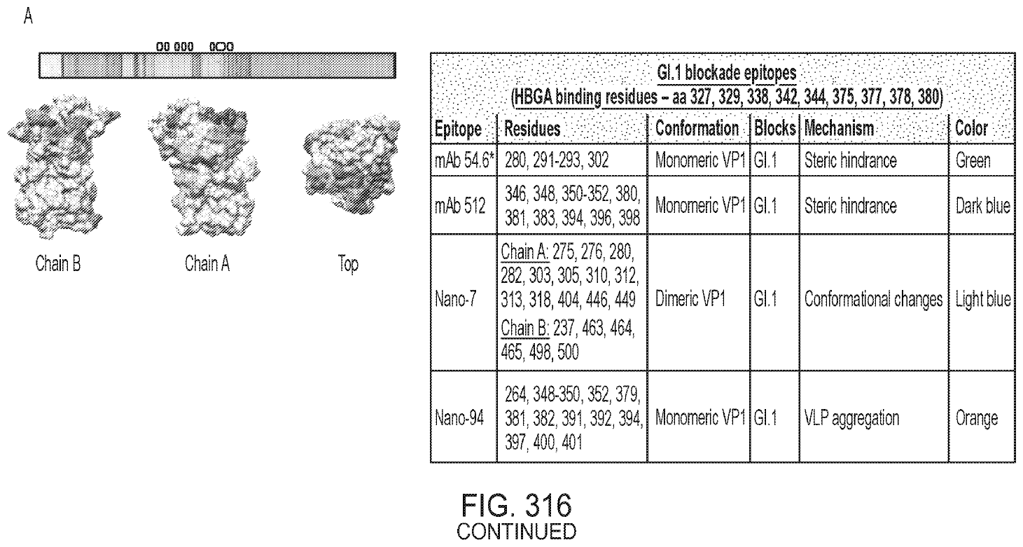

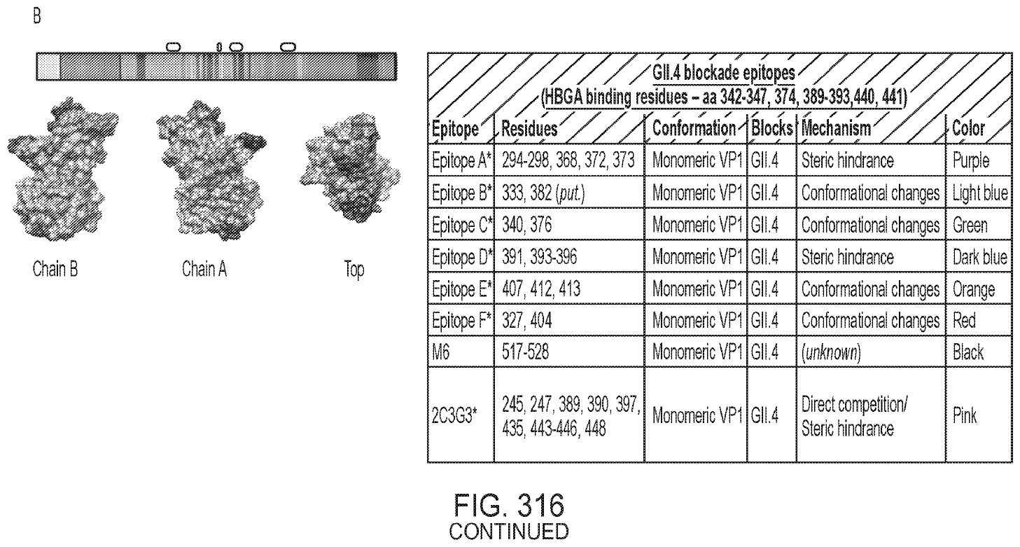

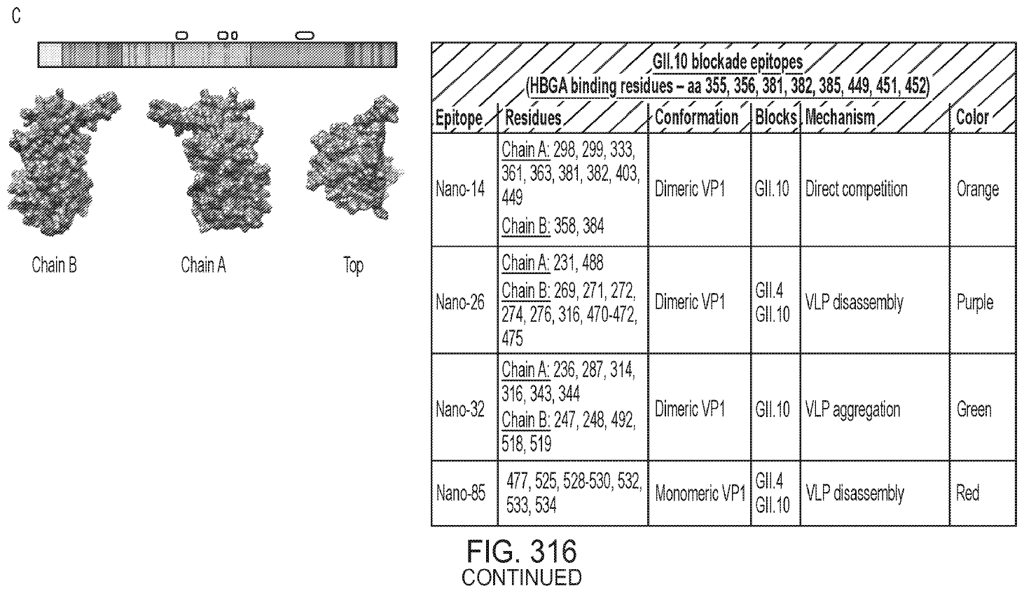

An exemplary schematic showing the norovirus lifecycle is included in FIG. 315. In more detail, however, infection by a norovirus begins when the viral capsid binds to a host cell surface receptor; histo-blood group antigens (HBGA) have been described as potential receptors or co-receptors. HBGAs are polymorphic glucans found on the surfaces of red blood cells and of certain epithelial cells (de Graaf et al. Nat Rev Microbiol. 14:421, 2016, doi: 10.1038/nrmicro.2016.48; Mallagaray et al. Nat Commun. 10:1320, 2019, doi: 10.1038/s41467-019-09251-5, each of which is incorporated herein by reference in its entirety). It has been reported that individuals who do not express fucosyltransferase 2 (Fut2), which generates HBGAs, are not susceptible to norovirus infection (de Graaf et al. Nat Rev Microbiol. 14:421, 2016, doi: 10.1038/nrmicro.2016.48, which is incorporated herein by reference in its entirety). Moreover, studies have shown that noroviruses recognize a determined group of HBGAs (Huang et al. J Virol. (2005) 79:6714, 2005, doi: 10.1128/JVI.79.11.6714-6722.2005, which is incorporated herein by reference in its entirety); at least four different binding patterns of human noroviruses have been described based on ABO blood type, Lewis blood group, and fut2 status (secretor/nonsecretor) (Huang et al. J Infect Dis. 188:19, 2003, doi: 10.1086/375742, which is incorporated herein by reference in its entirety). Generally, noroviruses that have HBGA type A/B binding patterns recognize the A and/or B and H antigens, but not the Lewis antigens; and noroviruses that have Lewis binding patterns bind only to Lewis antigens and/or the H antigen (Huang et al. J Virol. 79:6714, 2005, doi: 10.1128/JVI.79.11.6714-6722.2005, which is incorporated herein by reference in its entirety).

After binding, virus becomes internalized, uncoated, and disassembled; host factors are recruited to replicate and translate the genome (reviewed in de Graaf et al., Nat Rev Microbiol. 14:421, 2016, which is incorporated herein by reference in its entirety).

The genomes of noroviruses that infect humans comprise a linear, positive-sense RNA strand about 7.3-8.3 kb long (often about 7.5-7.7 kb). The 5′ end of the norovirus genome is covalently linked to one of the nonstructural proteins (the VPg protein) it encodes; the 3′ end is polyadenylated.

Upon internalization, the viral genome is released from the VPg protein, which then recruits host translation initiation factors (e.g., eIF3) and initiates assembly of the translation complex.

As described in more detail below, translation produces three proteins: the structural VP1 and VP2 proteins, and a polyprotein that is autocleaved to produce six (6) non-structural viral proteins, via a cascade that first generates three protein precursors, each of which becomes cleaved into two viral proteins.

Replication proceeds by transcribing the (+-strand) genome to generate (− strand) RNAs that become templates for synthesis of new (+-strand) genomic and subgenomic RNAs. These subgenomic RNAs contain the ORFs for VP1 and VP2, and are translated to produce these proteins. Replicated genomic RNAs are assembled into new virions that are released from the infected host cells.

### Genome

The norovirus genome includes short untranslated regions (UTRs) at either end; these contain evolutionarily conserved structures that are thought to participate in replication, translation, and/or pathogenesis.

The norovirus genome includes three open reading frames (ORFs 1, 2, and 3) that together encode eight viral proteins (reviewed in, Robilotti et al., Clin Microbiol Rev. 28:134, 2015, which is incorporated herein by reference in its entirety). ORF-2 and ORF-3 encode the structural components of the virion, viral protein 1 (VP1) and VP2, respectively. ORF-1 encodes the above-mentioned polyprotein that is proteolytically processed into the six nonstructural proteins of the virus: p48 (NA1/NS2), NTPase (NS3), p22(NS4), VPg (N5), Pro (NS6), and Pol (NS7; RdRp), these last two being the norovirus protease and RNA-dependent RNA polymerase, respectively. See, review of norovirus proteins in Compillay-Veliz et al. Front Immunol 11:961, 2020, which is incorporated herein by reference in its entirety)

VP1 is the primary structural protein of the capsid; 90 dimers of VP1 assemble into the icosahedral (T=3) capsid, with only a few copies of VP2 included. VP1 includes a shell (S) domain and a protruding (P) domain, with P1 and P2 components (see, for example, Prasad et al., Science 286:287, 1999, doi: 10.1126/science.286.5438.287, which is incorporated herein by reference in its entirety). The S domain makes up the core of the capsid, from which the P domain protrudes. The S domain is involved in binding VP2, thereby associating it with the capsid. The P domain, and particularly, P2, mediates binding to host HBGA molecules (see, e.g., Campillay-Veliz el al., Front. Immunol. 11:961, 2020, doi:10.3389/fnmu.2020.00961, which is incorporated herein by reference in its entirety). The P domain also mediates interactions between VP1 proteins and therefore impacts size and stability of viral capsids.

The S domain is located in the N-terminal portion of the VP1 protein, for example extending from about residue 225 to the end, according to canonical numbering systems. The P1 domain is typically considered to begin at residue 226 according to canonical numbering systems, and to be interrupted by the P2 domain, so that P1 includes residues 226-278 and 406-52, and P2 includes residues 278-406 according to canonical numbering systems.

The P2 subdomain is the most variable region of the VP1 protein, and is believed to be surface exposed on the viral capsid. P2 variants have been reported to be associated with particular epidemic outbreaks (see, for example, 22). The Pro protein is responsible for cleaving the polyprotein generated by translation of ORF1, first into p48/NTPase, p22/VPg and Pro/Pol precursor proteins, and ultimately into the six individual proteins.

The Pol, VPg, NTPase and p48 proteins have all been reported to play roles in viral replication. NTPase has been reported to have helicase, NTP hydrolase, and chaperone activities; p48 has been reported to increase Pol activity, and also disassembly of the trans-Golgi network, resulting in interference with host cell signaling pathways involved in immune response.

P22 has also been reported to contribute to trans-Golgi disassembly (36), and also to facilitate virion release from cells.

At least ten (10) different genogroups (GI-GX) of noroviruses have been defined (see, for example, Chhabra et al. J Gen Virol. 100:1393-406, 2019, doi: 10.1099/jgv.0.001318; see also, Campillay-Veliz et al., Front. Immunol, 11:961, 2020, doi: 10.3389/fimmu.2020.00961, each of which is incorporated herein by reference in its entirety) based on similarity of highly-conserved regions of either the Pol (NS7; RdRp) protein or (ii) VP1 (e.g., the amino acidic regions of VP1, such as are found in the S domain); three of these genogroups (specifically, GI, GII, and GIV) infect and cause acute gastroenteritis in humans. Norovirus genogroups have been further subdivided into genotypes, which in turn include strains and variants (e.g., that arise by mutation). Recombination between or among variants also gives rise to new strains (see, for example, Cannon J Virol. 83:5363, 2009, doi: 10.1128/JVI.02518-08; see also Vinje J Infect Dis. 176:1374, 1997, doi: 10.1086/517325, each of which is incorporated herein by reference in its entirety). The GII.4 genotype is the most prevalent worldwide; its Sydney and New Orleans variants are particularly prevalent (see, Glass et al N Engl J Med. 361:1776, 2009. doi: 10.1056/NEJMra0804575; Vinje et al. J Infect Dis. 176:1374, 1997, doi: 10.1086/517325; Tamminen et al Viruses. 11:91, 2019 doi: 10.3390/v11020091, each of which is incorporated herein by reference in its entirety) and a GII.P16-GII.4 Sydney recombinant strain was responsible for a 2015 pandemic (see, Lindesmith et al. J Infect Dis. 217:1145, 2017, doi: 10.1093/infdis/jix651, which is incorporated herein by reference in its entirety). Other highly infectious genotypes include GI.17 (see, for example, Lindesmith et al. J Infect Dis. 217:1145, 2017, doi: 10.1093/infdis/jix651; see also, Lindesmith et al. J Infect Dis. 216:1227, 2017, doi: 10.1093/infdis/jix385, each of which is incorporated herein by reference in its entirety).

To give an example of norovirus genogrouping, a system has been described in which viruses whose VP1 protein sequences differ by less than 14.3% are classified in the same strain; those whose VP1 protein sequences differ by 14.3-43.8% are classified in the same genotype, and those whose VP1 protein sequences differ between 45-61.4% are classified in the same genogroup (see Zheng et al. Virology 346:312, 2006, doi: 10.1016/j.virol.2005.11.015, which is incorporated herein by reference in its entirety).

The present disclosure (see, for example, Example 29), includes clade analysis of norovirus strain variants. In particular, as described herein, pairwise strain homologies were assessed by comparing the overlap of 9mer substrings of the constituent proteins; for a pair of strains, the number of 9mers present in both strains (the intersection) was divided by the number of 9mers present in at least one strain (the union) to derived a conservation score between 0 (no homology) and 1 (perfect homology). These pairwise similarities were used to inform a hierarchical cluster analysis that grouped strains according to their sequence similarity; these groups are referred to as “clades”. Annotations of the individual strains per clade were used to define labels for the clades, which include GI, GII.P2, GII.P4, GII.P7, GII.P12, GII.P16, GII.P17, and GIX. A small number of strains (<5%) were not assigned to any clade.

For each clade an exemplar sequence was selected. The exemplar was selected based on having a complete and clear gene annotation and having strong (e.g., better than average) homology (per the 9mer overlap approach described above) to the other strains in the clade. In some embodiments, the present disclosure provides and/or utilizes compositions (e.g., pharmaceutical compositions, e.g., immunogenic compositions, e.g., vaccines) that comprise or deliver one or more antigens from a particular norovirus clade as described herein. In some embodiments, the present disclosure provides and/or utilizes compositions (e.g., pharmaceutical compositions, e.g., immunogenic compositions, e.g., vaccines) that comprise or deliver one or more antigens from each of a plurality of norovirus clades as described herein; in some such embodiments, provided compositions (e.g., pharmaceutical compositions, e.g., immunogenic compositions, e.g., vaccines) may comprise or deliver separate antigens of different clades whereas in some embodiments provided compositions (e.g., pharmaceutical compositions, e.g., immunogenic compositions, e.g., vaccines) may comprise or deliver at least one antigen that includes epitopes from different norovirus clades as described herein.

For example, in some embodiments, provided compositions (e.g., pharmaceutical compositions, e.g., immunogenic compositions, e.g., vaccines) may comprise an RNA encoding a polypeptide (so that administration of such composition delivers the polypeptide) comprising epitope(s) of a single norovirus protein from a single norovirus clade; alternatively or additionally, in some embodiments, provided compositions (e.g., pharmaceutical compositions, e.g., immunogenic compositions, e.g., vaccines) may comprise an RNA encoding a polypeptide (so that administration of such composition delivers the polypeptide) that comprises one or more epitopes from each of two or more norovirus proteins of a single clade (e.g., of a single strain and/or a single variant or, alternatively of multiple strain(s) and/or variant(s) within the clade).

In some particular embodiments, provided compositions (e.g., pharmaceutical compositions, e.g., immunogenic compositions, e.g., vaccines) may comprise multiple RNAs, each of which encodes a polypeptide (so that administration of such composition delivers two or more polypeptides) comprising epitope(s) from a single norovirus clade (e.g., from a single strain and/or a single variant from the clade or alternatively from multiple strain(s) and/or variant(s) within the clade). In one particular such embodiment, a first RNA encodes a single protein or portion thereof and at least one second RNA encodes a string polypeptide (e.g., including multiple individual epitopes, optionally from different proteins, linked together in an artificial construct as described herein).

In alternative particular embodiments, provided compositions (e.g., pharmaceutical compositions, e.g., immunogenic compositions, e.g., vaccines) may comprise multiple RNAs, each of which encodes a polypeptide (so that administration of such composition delivers two or more polypeptides) comprising epitope(s) from a single norovirus clade (e.g., from a single strain and/or a single variant from the clade or alternatively from multiple strain(s) and/or variant(s) within the clade), or from multiple norovirus clades such that, together, the polypeptides comprise epitopes from multiple clades. In one particular such embodiment, at least one first RNA encodes a single protein or portion thereof and at least one second RNA encodes a string polypeptide (e.g., including multiple individual epitopes, optionally from different proteins, linked together in an artificial construct as described herein).

It has been reported that individuals infected with norovirus of one genogroup do not typically develop immunity to other genogroups (see, e.g., Esposito and Principi, Front. Immunol. 11: 1383, 2020; see also, Atmar et al., Curr. Opin. Infect. Dis. 31: 422 (2018); and Brown et al. J. Clin. Virol. 96:44 (2017), each of which is incorporated herein by reference in its entirety) even when they may develop immunity to other strains or variants within the genogroup with which they were infected. In some embodiments, as described herein, provided technologies administer or deliver (e.g., by administration of an encoding RNA) polypeptides that, together, are or comprise epitopes from multiple genotypes (e.g., GI and GII) and/or multiple clades as described herein.

In some embodiments (e.g., including those specific embodiments described immediately above), the present disclosure provides and/or utilizes compositions (e.g., pharmaceutical compositions, e.g., immunogenic compositions, e.g., vaccines) that comprise or deliver one or more GII antigens (e.g., that are or comprise GII proteins or fragments or epitopes thereof), such as one or more GII.4 antigens. Alternatively or additionally, in some embodiments, (e.g., including those specific embodiments described immediately above), the present disclosure provides and/or utilizes compositions (e.g., pharmaceutical compositions, e.g., immunogenic compositions, e.g., vaccines) that comprise or deliver one or more GI antigens (e.g., that are or comprise GI proteins or fragments or epitopes thereof). In some particular embodiments, (e.g., including those specific embodiments described immediately above), the present disclosure provides and/or utilizes compositions (e.g., pharmaceutical compositions, e.g., immunogenic compositions, e.g., vaccines) that comprise or deliver one or more GII antigens (e.g., that are or comprise GII proteins or fragments or epitopes thereof), such as one or more GII.4 antigens, and also one or more GI antigens (e.g., that are or comprise GI proteins or fragments or epitopes thereof).

In some embodiments, (e.g., including those specific embodiments described immediately above), a utilized antigen is or comprises a VP protein, such as a VP1 protein, or a fragment or epitope thereof (e.g., of an S domain and/or a P domain, such as a P2 domain), e.g., as described herein.

### Antigens

The present disclosure provides pharmaceutical compositions (e.g., immunogenic compositions, e.g., vaccines) that deliver particular viral antigen constructs to a subject (e.g., a patient) and related technologies (e.g., methods).

In some embodiments, the present disclosure provides certain viral antigen constructs particularly useful in effective vaccination.

Antigens utilized in accordance with the present disclosure are or include viral components (e.g., proteins or fragments or epitopes thereof, including epitopes that may comprise non-amino acid, e.g., carbohydrate moieties), which components induce immune responses when administered to humans (or other animals such as rodents and non-human primates susceptible to viral infection).

In many embodiments, antigens utilized in provided pharmaceutical compositions (e.g., immunogenic compositions, e.g., vaccines) include both B-cell and T-cell epitopes, as described herein. In some particular embodiments, delivered antigens include both B-cell and CD4 T cell epitopes, optionally together in a single antigen polypeptide. In some embodiments, antigens utilized in provided pharmaceutical compositions (e.g., immunogenic compositions, e.g., vaccines) include CD8 T cell epitopes. In some embodiments, antigens utilized in provided pharmaceutical compositions (e.g., immunogenic composition, e.g., vaccine), together, include B cell, CD4 T cell and CD8 T cell epitopes. Indeed, in some embodiments, the present disclosure defines particularly useful epitopes for inclusion in viral vaccines, and/or provides antigens that include them.

Exemplary viral antigens can be VZV antigens, and the present disclosure provides pharmaceutical compositions (e.g., immunogenic compositions, e.g., vaccines) that deliver particular VZV antigen constructs to a subject (e.g., a patient) and related technologies (e.g., methods).

In some embodiments, the present disclosure provides certain VZV antigen constructs particularly useful in effective vaccination.

Antigens utilized in accordance with the present disclosure are or include VZV components (e.g., proteins or fragments or epitopes thereof, including epitopes that may comprise non-amino acid, e.g., carbohydrate moieties), which components induce immune responses when administered to humans (or other animals such as rodents and non-human primates susceptible to VZV infection).

In many embodiments, antigens utilized in provided pharmaceutical compositions (e.g., immunogenic compositions, e.g., vaccines) include both B-cell and T-cell epitopes, as described herein. In some particular embodiments, delivered antigens include both B-cell and CD4 T cell epitopes, optionally together in a single antigen polypeptide. In some embodiments, antigens utilized in provided pharmaceutical compositions (e.g., immunogenic compositions, e.g., vaccines) include CD8 T cell epitopes. In some embodiments, antigens utilized in provided pharmaceutical compositions (e.g., immunogenic composition, e.g., vaccine), together, include B cell, CD4 T cell and CD8 T cell epitopes. Indeed, in some embodiments, the present disclosure defines particularly useful epitopes for inclusion in VZV vaccines, and/or provides antigens that include them.

Exemplary VZV antigens and/or epitopes for use in compositions described herein can be found in, e.g., Table 3A, Table 3B, and/or Table 4A herein. In some embodiments, exemplary VZV antigens and/or epitopes encoded by genes as disclosed in Tables 1A-1I and/or Tables 2A-2B can be useful for compositions described herein.

Exemplary viral antigens can be CMV antigens, and the present disclosure provides pharmaceutical compositions (e.g., immunogenic compositions, e.g., vaccines) that deliver particular CMV antigen constructs to a subject (e.g., a patient) and related technologies (e.g., methods).

In some embodiments, the present disclosure provides certain CMV antigen constructs particularly useful in effective vaccination.

Antigens utilized in accordance with the present disclosure are or include CMV components (e.g., proteins or fragments or epitopes thereof, including epitopes that may comprise non-amino acid, e.g., carbohydrate moieties), which components induce immune responses when administered to humans (or other animals such as rodents and non-human primates susceptible to CMV infection).

In many embodiments, antigens utilized in provided pharmaceutical compositions (e.g., immunogenic compositions, e.g., vaccines) include both B-cell and T-cell epitopes, as described herein. In some particular embodiments, delivered antigens include both B-cell and CD4 T cell epitopes, optionally together in a single antigen polypeptide. In some embodiments, antigens utilized in provided pharmaceutical compositions (e.g., immunogenic compositions, e.g., vaccines) include CD8 T cell epitopes. In some embodiments, antigens utilized in provided pharmaceutical compositions (e.g., immunogenic composition, e.g., vaccine), together, include B cell, CD4 T cell and CD8 T cell epitopes. Indeed, in some embodiments, the present disclosure defines particularly useful epitopes for inclusion in CMV vaccines, and/or provides antigens that include them.

Exemplary CMV antigens and/or epitopes for use in compositions described herein can be found in, e.g., Table 8A, Table 8B, and/or Table 9A herein. In some embodiments, exemplary CMV antigens and/or epitopes encoded by genes as disclosed in Tables 6A-6F and/or Tables 7A-7B can be useful for compositions described herein.

Exemplary viral antigens can be norovirus antigens, and the present disclosure provides pharmaceutical compositions (e.g., immunogenic compositions, e.g., vaccines) that deliver particular norovirus antigen constructs to a subject (e.g., a patient) and related technologies (e.g., methods).

In some embodiments, the present disclosure provides certain norovirus antigen constructs particularly useful in effective vaccination.

Antigens utilized in accordance with the present disclosure are or include norovirus components (e.g., proteins or fragments or epitopes thereof, including epitopes that may comprise non-amino acid, e.g., carbohydrate moieties), which components induce immune responses when administered to humans (or other animals such as rodents and non-human primates susceptible to norovirus infection).

In many embodiments, antigens utilized in provided pharmaceutical compositions (e.g., immunogenic compositions, e.g., vaccines) include both B-cell and T-cell epitopes, as described herein. In some particular embodiments, delivered antigens include both B-cell and CD4 T cell epitopes, optionally together in a single antigen polypeptide. In some embodiments, antigens utilized in provided pharmaceutical compositions (e.g., immunogenic compositions, e.g., vaccines) include CD8 T cell epitopes. In some embodiments, antigens utilized in provided pharmaceutical compositions (e.g., immunogenic composition, e.g., vaccine), together, include B cell, CD4 T cell and CD8 T cell epitopes. Indeed, in some embodiments, the present disclosure defines particularly useful epitopes for inclusion in norovirus vaccines, and/or provides antigens that include them.

Exemplary Norovirus antigens and/or epitopes for use in compositions described herein can be found in, e.g., Tables 14A-14N and/or Table 15A herein. In some embodiments, exemplary Norovirus antigens and/or epitopes encoded by genes as disclosed in Tables 12A-12D and/or Tables 13A-13B can be useful for compositions described herein.

### Protein Sequences

In some embodiments, a provided pharmaceutical composition (e.g., immunogenic composition, e.g., viral vaccine) comprises or delivers (e.g., causes expression of in a recipient organism, for example by administration of a nucleic acid construct, such as an RNA construct as described herein, that encodes it) an antigen that is or comprises one or more epitopes (e.g., one or more B-cell and/or one or more T-cell epitopes) of a viral protein. In some embodiments, a pharmaceutical composition described herein induces a relevant immune response effective against virus (e.g., by targeting a viral protein).

In some embodiments a provided pharmaceutical composition (e.g., immunogenic composition, e.g., viral vaccine) comprises or delivers an antigen that is or comprises a full-length viral protein. In some embodiments, a provided pharmaceutical composition (e.g., immunogenic composition, e.g., viral vaccine) comprises or delivers an antigen that is or comprises a portion of a viral protein that is less than a full-length viral protein. In some embodiments, a provided pharmaceutical composition (e.g., immunogenic composition, e.g., viral vaccine) comprises or delivers a chimeric polypeptide that is or comprises part or all of a viral protein and one or more heterologous polypeptide elements.

In some embodiments, an antigen that is included in and/or delivered by a provided pharmaceutical composition (e.g., immunogenic composition, e.g., viral vaccine) is or comprises one or more peptide fragments of a viral antigen; in some such embodiments, each of the one or more peptide fragments includes at least one epitope (e.g., one or more B cell epitopes and/or one or more T cell epitopes), for example as may be predicted, selected, assessed and/or characterized as described herein.

In some embodiments, an antigen that is included in and/or delivered by a provided pharmaceutical composition (e.g., immunogenic composition, e.g., viral vaccine) is or comprises a plurality of peptide fragments of one or more viral antigens. In some embodiments, a single polypeptide antigen may include a plurality of such fragments, e.g., presented as a string antigen as described herein.

In some embodiments, one or more viral epitopes (e.g., included in part or all of a viral protein) may be linked with one or more sequences with which it is linked in nature; in some such embodiments, such sequence(s) may be or comprise one or more heterologous elements (e.g., one or more elements, not naturally found in the relevant viral protein or not naturally found directly linked to the relevant viral epitope(s)). For example, in some embodiments, an antigen peptide provided and/or utilized in accordance with the present disclosure may include one or more linker elements and/or one or more membrane association elements and/or one or more secretion elements, etc. In some embodiments, an antigen peptide may comprise a plurality of viral protein fragments or epitopes separated from one another by linkers.

In some embodiments, a viral protein, or fragment or epitope thereof, utilized in an antigen as described herein may include one or more sequence alterations relative to a particular reference viral protein, or fragment or epitope thereof. For example, in some embodiments, a utilized antigen may include one or more sequence variations found in circulating strains or predicted to arise, e.g., in light of assessments of sequence conservation and/or evolution of viral proteins over time and/or across strains. Alternatively or additionally, in some embodiments, a utilized antigen may include one or more sequence variations selected, for example, to impact stability, folding, processing and/or display of the antigen or any epitope thereof.

In some embodiments, a viral protein, or fragment or epitope thereof, utilized in an antigen as described herein shows at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with a relevant corresponding reference (e.g., wild type) protein, fragment or epitope. In some embodiments, a viral protein, or fragment or epitope thereof, utilized in an antigen as described herein shows at least 80%, 81%, 82%, 83%, 84%, 85%, 86%, 87%, 88%, 89%, 90%, 91%, 92%, 93%, 94%, 95%, 96%, 97%, 98%, 99%, or 100% sequence homology (i.e., identity or conservative substitution as is understood in the art) amino acid sequence identity with a relevant corresponding reference (e.g., wild type) protein, fragment or epitope. Moreover, in some embodiments, a viral protein, or fragment or epitope thereof, utilized in an antigen as described herein shares conserved amino acid residues (e.g., at corresponding positions) with a relevant corresponding reference (e.g., wild type) protein, fragment or epitope. Those skilled in the art will appreciate that, in general, lower percent identity or homology may be tolerated for shorter peptides, as a single change will by definition have a larger impact on percent identity or homology when considered relative to a smaller number of residues. For example, those skilled in the art will appreciate that, for sequences longer than about 20 amino acids, percent identity or homology is typically greater than about 80%; for sequences longer than about 50 amino acids, percent identity or homology is typically greater than about 90%.

In some embodiments, assessments of degree of conservation may consider the physiochemical difference between two amino acids as described, for example, in WO2014/180569. It is well known in molecular evolution that amino acids that interchange frequently are likely to have chemical and physical similarities whereas amino acids that interchange rarely are likely to have different physico-chemical properties. The likelihood for a given substitution to occur in nature compared with the likelihood for this substitution to occur by chance can measured by log-odds matrices. The patterns observed in log-odds matrices imposed by natural selection “reflect the similarity of the functions of the amino acid residues in their weak interactions with one another in the three dimensional conformation of proteins” (See Dayhoff et al. Atlas of protein sequence and structure 5:345, 1978). In some embodiments, evolutionary based log-odds matrices, which may be referred to as “T scores” can be used to reflect extent to which a sequence variation might impact T cell recognition. Substitutions with positive T scores (i.e., log-odds) are likely to occur in nature, and hence correspond to two amino acids that have similar physico-chemical properties. Substitutions with positive T scores would have a lower likelihood of altering immunogenicity. Conversely, substitutions with negative T scores reflect substitutions that are unlikely to occur in nature and hence correspond to two amino acids that have significantly different physico-chemical properties. Such substitutions would have a greater chance of altering immunogenicity. In some embodiments, presence of negative T score substitutions within a sequence, even if it is otherwise highly conserved, may indicate that it would be relatively less useful in a vaccine antigen as described herein.

In some embodiments, a utilized antigen is or comprises one or more viral protein sequences (e.g., conserved sequences and/or sequences that are or comprise one or more B cell epitopes and/or one or more CD4 epitopes and/or one or more CD8 epitopes) of an antigen.

In some embodiments, a utilized antigen is or comprises one or more viral protein sequences found in a strain that is circulating or has circulated in a relevant region (e.g., where subjects to be vaccinated are or will be present).

In some embodiments, an antigen utilized in accordance with the present disclosure includes viral protein sequences identified and/or characterized by one or more of:
HLA-I or HLA-II binding (e.g., to HLA allele(s) present in a relevant population)
HLA ligandomics data, optionally confirmed by mass spectrometry
Relatively high expression
Sequence conservation
Surface exposure
Serum reactivity
Immunogenicity (e.g., presence of one or more B-cell and/or T-cell epitopes; evidence of ability to induce sterile protection in model systems including, e.g., humans, non-human primates, and/or mice).
Absence of sequences that overlap with human proteome

In some embodiments, such characteristics are experimentally or computationally assessed. In some embodiments, such characteristics are assessed by consultation with published reports.

For example, in some embodiments, HLA-I and/or HLA-II binding is experimentally assessed; in some embodiments it is predicted.

In some embodiments, predicted HLA-I or HLA-II binding is assessed using an algorithm such as neonmhc 1 and/or neonmhc2, which predict and/or characterize likelihood of MHC class I and MHC class II binding, respectively. Alternatively or additionally, in some embodiments, an MHC-peptide presentation prediction algorithm or MHC-peptide presentation predictor is or comprises NetMHCpan or NetMICIIpan. In some embodiments, a hidden markov model approach may be utilized for MHC-peptide presentation prediction and/or characterization. In some embodiments, the peptide prediction model MARIA may be utilized. In some embodiments, NetMHCpan is not utilized to predict or characterize likelihood of MHC binding for peptides as described herein. In some embodiments, the peptide prediction model MARIA may be utilized. In some embodiments, NetMHCIIpan is not utilized to predict or characterize likelihood of MHC binding for peptides as described herein. In some embodiments, neither NetMHCpan nor NetMICIIpan is utilized to predict or characterize likelihood of MHC binding for peptides as described herein. In some embodiments, an MHC-peptide presentation prediction algorithm or MHC-peptide presentation predictor is or comprises RECON® (Real-time Epitope Computation for ONcology), which offers high quality MHC-peptide presentation prediction based on expression, processing and binding capabilities. See, for example, Abelin et al., Immunity 21:315, 2017; Abelin et al., Immunity 15:766, 2019.

In some embodiments, HLA binding and/or ligandomics assessments will consider the geographic region of subjects to be immunized. For example, in some embodiments, HLA allelic diversity will be considered. In some embodiments, antigen(s) included in a provided pharmaceutical composition (e.g., immunogenic composition, e.g., vaccine) will be or comprise peptides (e.g., epitopes) expected or determined, when considered together, to bind to a significant percentage (e.g., at least 60%, 65%, 70%, 75%, 80%, 85%, 90%, 95%, or more) of HLA alleles expected or known to be present in a relevant region or population. In some embodiments, antigen(s) included in a provided pharmaceutical composition (e.g., immunogenic composition, e.g., vaccine) will be or comprise peptides expected or determined, when considered together, to bind to the most prevalent (e.g., the 1, 2, 3, 4, 5, 6, 7, 8, 9, or 10 most prevalent, or at least 1, 2, 3, 4, or 5 of the 10 most prevalent, etc.) HLA alleles expected or known to be present in a relevant region or population).

In some embodiments, expression level is experimentally determined (e.g., in a model system or in infected humans). In some embodiments, expression level is a reported level (e.g., in a published or presented report). In some embodiments, expression level is assessed as RNA (e.g., via RNASeq). In some embodiments (and typically preferably), expression levels is assessed as protein.

In some embodiments, sequence conservation is assessed, for example, using publicly available sequence evaluation software (such as, for example, multiple sequence alignment programs MAFFT, Clustal Omega, etc.). In some embodiments, sequence conservation is determined by consultation with published resources (e.g., sequences). In some embodiments, sequence conservation includes consideration of currently or recently detected strains (e.g., in an active outbreak).

In some embodiments, surface exposure is assessed by reference to publicly available database and/or software. In some embodiments, surface exposure is assessed by reference to publicly available data.

In some embodiments, serum reactivity is assessed by contacting serum samples from infected individuals with polypeptides including sequences of interest (e.g., as may be displayed via, for example, phage display or peptide array, etc; see, for example, Whittemore et al “A General Method to Discover Epitopes from Sera” PlosOne, 2016; https://doi.org/10.1371/journal.pone.0157462). In some embodiments, serum reactivity is assessed by consultation with literature reports and or database data indicating serum-recognized sequences.

In some embodiments, assessment of immunoreactivity and/or of presence of an epitope may be or comprise consultation with the Immune Epitope Database (IEDB) which those skilled in the art will be aware is a freely available resource funded by NIAID that catalogs experimental data on antibody and T cell epitopes (see iedb.org).

In some embodiments, ability to induce sterile protection is assessed. In some embodiments, antigen(s) utilized in accordance with the present disclosure are characterized by dendritic cell presentation which, in turn may be indicative of HLA binding and/or of immunogenicity. In some embodiments, antigen(s) utilized in accordance with the present disclosure are or comprises sequences (e.g., epitopes, fragments, complete proteins) of viral proteins expressed prior to infection or introduction into a cell, although in some embodiments, antigens localized to the surface of infected host-cells and/or during the intracellular life cycle in either the active or latent stage may also be included.

In some embodiments, an antigen utilized in accordance with the present disclosure an antigen is or comprises a viral protein or variant thereof or one or more fragments or epitopes of such thereof (e.g., used individually or in combination (e.g., as part of a multiepitope construct, such as a string construct, as described herein) with one another and/or with one or more other viral proteins or fragments or epitopes thereof).

Among other things, the present disclosure provides an insight that, in some embodiments, it may be desirable to include two or more different epitopes, optionally from two or more different viral proteins, in pharmaceutical composition (e.g., immunogenic composition, e.g., vaccine) compositions.

### VZV Protein Sequences

In some embodiments, a provided composition (e.g., a pharmaceutical composition, e.g., immunogenic composition, e.g., vaccine) comprises or delivers (e.g., causes expression of in a recipient organism, for example by administration of a nucleic acid construct, such as an RNA construct as described herein, that encodes it) an antigen that is or comprises one or more epitopes (e.g., one or more B-cell and/or one or more T-cell epitopes) of a VZV protein. In some embodiments, a composition described herein induces a relevant immune response effective against VZV (e.g., by targeting a VZV protein).

In some embodiments, a provided composition (e.g., a pharmaceutical composition, e.g., immunogenic composition, e.g., vaccine) comprises or delivers an antigen that is or comprises a full-length VZV protein. In some embodiments, a provided composition (e.g., a pharmaceutical composition, e.g., immunogenic composition, e.g., vaccine) comprises or delivers an antigen that is or comprises a portion of a VZV protein that is less than a full-length VZV protein. In some embodiments, a provided composition (e.g., a pharmaceutical composition, e.g., immunogenic composition, e.g., vaccine) comprises or delivers a chimeric polypeptide that is or comprises part or all of a VZV protein and one or more heterologous polypeptide elements.

In some embodiments, an antigen that is included in and/or delivered by a provided pharmaceutical composition (e.g., immunogenic composition, e.g., VZV vaccine) is or comprises one or more peptide fragments of a VZV antigen; in some such embodiments, each of the one or more peptide fragments includes at least one epitope (e.g., one or more B cell epitopes and/or one or more T cell epitopes), for example as may be predicted, selected, assessed and/or characterized as described herein.

In some embodiments, an antigen that is included in and/or delivered by a provided pharmaceutical composition (e.g., immunogenic composition, e.g., VZV vaccine) is or comprises a plurality of peptide fragments of one or more VZV antigens. In some embodiments, a single polypeptide antigen may include a plurality of such fragments, e.g., presented as a string antigen as described herein.

In some embodiments, one or more VZV epitopes (e.g., included in part or all of a VZV protein) may be linked with one or more sequences with which it is linked in nature; in some such embodiments, such sequence(s) may be or comprise one or more heterologous elements (e.g., one or more elements, not naturally found in the relevant VZV protein or not naturally found directly linked to the relevant VZV epitope(s)). For example, in some embodiments, an antigen peptide provided and/or utilized in accordance with the present disclosure may include one or more linker elements and/or one or more membrane association elements and/or one or more secretion elements, etc. In some embodiments, an antigen peptide may comprise a plurality of VZV protein fragments or epitopes separated from one another by linkers.

In some embodiments, a VZV protein, or fragment or epitope thereof, utilized in an antigen as described herein may include one or more sequence alterations relative to a particular reference VZV protein, or fragment or epitope thereof. For example, in some embodiments, a utilized antigen may include one or more sequence variations found in circulating strains or predicted to arise, e.g., in light of assessments of sequence conservation and/or evolution of VZV proteins over time and/or across strains. Alternatively or additionally, in some embodiments, a utilized antigen may include one or more sequence variations selected, for example, to impact stability, folding, processing and/or display of the antigen or any epitope thereof.

In some embodiments, a VZV protein, or fragment or epitope thereof, utilized in an antigen as described herein shows at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with a relevant corresponding reference (e.g., wild type) protein, fragment or epitope. In some embodiments, a VZV protein, or fragment or epitope thereof, utilized in an antigen as described herein shows at least 80%, 81%, 82%, 83%, 84%, 85%, 86%, 87%, 88%, 89%, 90%, 91%, 92%, 93%, 94%, 95%, 96%, 97%, 98%, 99%, or 100% sequence homology (i.e., identity or conservative substitution as is understood in the art) amino acid sequence identity with a relevant corresponding reference (e.g., wild type) protein, fragment or epitope. Moreover, in some embodiments, a VZV protein, or fragment or epitope thereof, utilized in an antigen as described herein shares conserved amino acid residues (e.g., at corresponding positions) with a relevant corresponding reference (e.g., wild type) protein, fragment or epitope. Those skilled in the art will appreciate that, in general, lower percent identity or homology may be tolerated for shorter peptides, as a single change will by definition have a larger impact on percent identity or homology when considered relative to a smaller number of residues. For example, those skilled in the art will appreciate that, for sequences longer than about 20 amino acids, percent identity or homology is typically greater than about 80%; for sequences longer than about 50 amino acids, percent identity or homology is typically greater than about 90%.

In some embodiments, assessments of degree of conservation may consider the physiochemical difference between two amino acids as described, for example, in WO2014/180569, which is incorporated herein by reference in its entirety. It is well known in molecular evolution that amino acids that interchange frequently are likely to have chemical and physical similarities whereas amino acids that interchange rarely are likely to have different physico-chemical properties. The likelihood for a given substitution to occur in nature compared with the likelihood for this substitution to occur by chance can measured by log-odds matrices. The patterns observed in log-odds matrices imposed by natural selection “reflect the similarity of the functions of the amino acid residues in their weak interactions with one another in the three dimensional conformation of proteins” (see Dayhoff et al. Atlas of protein sequence and structure 5:345, 1978180569, which is incorporated herein by reference in its entirety). In some embodiments, evolutionary based log-odds matrices, which may be referred to as “T scores” can be used to reflect extent to which a sequence variation might impact T cell recognition. Substitutions with positive T scores (i.e., log-odds) are likely to occur in nature, and hence correspond to two amino acids that have similar physico-chemical properties. Substitutions with positive T scores would have a lower likelihood of altering immunogenicity. Conversely, substitutions with negative T scores reflect substitutions that are unlikely to occur in nature and hence correspond to two amino acids that have significantly different physico-chemical properties. Such substitutions would have a greater chance of altering immunogenicity. In some embodiments, presence of negative T score substitutions within a sequence, even if it is otherwise highly conserved, may indicate that it would be relatively less useful in a vaccine antigen as described herein.

In some embodiments, a utilized antigen induces an immune response that targets a VZV envelope glycoprotein. In some embodiments, one or more antigens induce an immune response that targets a VZV envelope glycoprotein. In some embodiments, one or more antigens comprises one or more VZV protein sequences (e.g., conserved sequences and/or sequences that are or comprise one or more B cell epitopes and/or one or more CD4 epitopes and/or one or more CD8 epitopes) of an antigen or epitope of a VZV envelope glycoprotein. In some embodiments, one or more antigens is or comprises a VZV gE protein or a fragment or epitope thereof. In some embodiments, one or more antigens is or comprises a VZV gB protein or a fragment or epitope thereof. In some embodiments, one or more antigens is or comprises a VZV gC protein or a fragment or epitope thereof. In some embodiments, one or more antigens is or comprises a VZV gI protein or a fragment or epitope thereof. In some embodiments, one or more antigens is or comprises a VZV gH protein or a fragment or epitope thereof. In some embodiments, one or more antigens is or comprises a VZV gL protein or a fragment or epitope thereof. In some embodiments, one or more antigens is or comprises a VZV gM protein or a fragment or epitope thereof. In some embodiments, one or more antigens is or comprises a VZV gN protein or a fragment or epitope thereof. In some embodiments, one or more antigens is or comprises one or more of a VZV gE protein, fragment or epitope thereof, a VZV gB protein, fragment or epitope thereof, a VZV gC protein, fragment or epitope thereof, a VZV gI protein, fragment or epitope thereof, a VZV gH protein, fragment or epitope thereof, a VZV gL protein, fragment or epitope thereof, a VZV gM protein, fragment or epitope thereof, a VZV gN protein, fragment or epitope thereof, or a combination thereof.

In some embodiments, a utilized antigen induces an immune response that targets a VZV tegument protein. In some embodiments, one or more antigens is or comprises a VZV UL21 protein, fragment or epitope thereof. In some embodiments, one or more antigens is or comprises a VZV VP22 protein, fragment or epitope thereof. In some embodiments, one or more antigens is or comprises a VZV large tegument protein, fragment or epitope thereof, or a combination thereof. In some embodiments, one or more antigens is or comprises one or more of a VZV UL21 protein, fragment or epitope thereof, a VZV VP22 protein, fragment or epitope thereof, a VZV large tegument protein, fragment or epitope thereof, or a combination thereof.

In some embodiments, one or more antigens is or comprises one or more antigens listed in Table 3A, Table 3B, and/or Table 4A herein. In some embodiments, one or more antigens is or comprises one or more antigens listed in Table 4A herein. In some embodiments, one or more antigens is or comprises one or more antigens listed in Table 4B herein. In some embodiments, one or more antigens is or comprises one or more antigens encoded by respective genes listed in Tables 1A-1I and/or Tables 2A-2B.

In some embodiments, one or more antigens in accordance with the present disclosure is or comprises a polypeptide or portion thereof encoded by all or part of ORF 4, ORF 9, ORF 10, ORF 12, ORF 18, ORF 19, ORF 22, ORF 24, ORF 27, ORF 28, ORF 29, ORF 31, ORF 34, ORF 36, ORF 37, ORF 38, ORF 41, ORF 48, ORF 50, ORF 53, ORF 59, ORF 62, ORF 63, ORF 67, ORF 68, or a combination thereof. In some embodiments, one or more antigens utilized in accordance with the present disclosure is or comprises a polypeptide or portion thereof encoded by all or part of ORF 9, ORF 12, ORF 18, ORF 19, ORF 24, ORF 27, ORF 29, ORF 36, ORF 37, ORF 38, ORF 41, ORF 48, ORF 50, ORF 59, ORF 62, ORF 63, ORF 67, ORF 68, or a combination thereof.

In some embodiments, an antigen utilized in accordance with the present disclosure includes VZV protein sequences identified and/or characterized by one or more of:
HLA-I or HLA-II binding (e.g., to HLA allele(s) present in a relevant population)
HLA ligandomics data, optionally confirmed by mass spectrometry
Relatively high expression
Sequence conservation
Surface exposure
Serum reactivity
Immunogenicity (e.g., presence of one or more B-cell and/or T-cell epitopes; evidence of ability to induce sterile protection in model systems including, e.g., humans, non-human primates, and/or mice).
Absence of sequences that overlap with human proteome

In some embodiments, such characteristics are experimentally or computationally assessed. In some embodiments, such characteristics are assessed by consultation with published reports.

For example, in some embodiments, HLA-I and/or HLA-II binding is experimentally assessed; in some embodiments it is predicted.

In some embodiments, predicted HLA-I or HLA-II binding is assessed using an algorithm such as neonmhc 1 and/or neonmhc2, which predict and/or characterize likelihood of MHC class I and MHC class II binding, respectively. Alternatively or additionally, in some embodiments, an MHC-peptide presentation prediction algorithm or MHC-peptide presentation predictor is or comprises NetMHCpan or NetMHCIIpan. In some embodiments, a hidden markov model approach may be utilized for MHC-peptide presentation prediction and/or characterization. In some embodiments, the peptide prediction model MARIA may be utilized. In some embodiments, NetMHCpan is not utilized to predict or characterize likelihood of MHC binding for peptides as described herein. In some embodiments, the peptide prediction model MARIA may be utilized. In some embodiments, NetMHCIIpan is not utilized to predict or characterize likelihood of MHC binding for peptides as described herein. In some embodiments, neither NetMHCpan nor NetMHCIIpan is utilized to predict or characterize likelihood of MHC binding for peptides as described herein. In some embodiments, an MHC-peptide presentation prediction algorithm or MHC-peptide presentation predictor is or comprises RECON® (Real-time Epitope Computation for ONcology), which offers high quality MHC-peptide presentation prediction based on expression, processing and binding capabilities. See, for example, Abelin et al., Immunity 21:315, 2017; Abelin et al., Immunity 15:766, 2019.

In some embodiments, HLA binding and/or ligandomics assessments will consider the geographic region of subjects to be immunized. For example, in some embodiments, HLA allelic diversity will be considered. In some embodiments, antigen(s) included in a provided pharmaceutical composition (e.g., immunogenic composition, e.g., vaccine) will be or comprise peptides (e.g., epitopes) expected or determined, when considered together, to bind to a significant percentage (e.g., at least 60%, 65%, 70%, 75%, 80%, 85%, 90%, 95%, or more) of HLA alleles expected or known to be present in a relevant region or population. In some embodiments, antigen(s) included in a provided pharmaceutical composition (e.g., immunogenic composition, e.g., vaccine) will be or comprise peptides expected or determined, when considered together, to bind to the most prevalent (e.g., the 1, 2, 3, 4, 5, 6, 7, 8, 9, or 10 most prevalent, or at least 1, 2, 3, 4, or 5 of the 10 most prevalent, etc.) HLA alleles expected or known to be present in a relevant region or population).

In some embodiments, expression level is experimentally determined (e.g., in a model system or in infected humans). In some embodiments, expression level is a reported level (e.g., in a published or presented report). In some embodiments, expression level is assessed as RNA (e.g., via RNASeq). In some embodiments (and typically preferably), expression levels is assessed as protein.

In some embodiments, sequence conservation is assessed, for example, using publicly available sequence evaluation software (such as, for example, multiple sequence alignment programs MAFFT, Clustal Omega, etc.). In some embodiments, sequence conservation is determined by consultation with published resources (e.g., sequences). In some embodiments, sequence conservation includes consideration of currently or recently detected strains (e.g., in an active outbreak).

In some embodiments, surface exposure is assessed by reference to publicly available database and/or software.

In some embodiments, serum reactivity is assessed by contacting serum samples from infected individuals with polypeptides including sequences of interest (e.g., as may be displayed via, for example, phage display or peptide array, etc; see, for example, Whittemore et al “A General Method to Discover Epitopes from Sera” PlosOne, 2016; https://doi.org/10.1371/journal.pone.0157462). In some embodiments, serum reactivity is assessed by consultation with literature reports and or database data indicating serum-recognized sequences.

In some embodiments, assessment of immunoreactivity and/or of presence of an epitope may be or comprise consultation with the Immune Epitope Database (IEDB) which those skilled in the art will be aware is a freely available resource funded by NIAID that catalogs experimental data on antibody and T cell epitopes (see iedb.org).

In some embodiments, antigen(s) utilized in accordance with the present disclosure are characterized by dendritic cell presentation which, in turn may be indicative of HLA binding and/or of immunogenicity.

In some embodiments, antigen(s) utilized in accordance with the present disclosure are or comprises sequences (e.g., epitopes, fragments, complete proteins) of VZV proteins found in the VZV envelope. In some embodiments, antigen(s) utilized in accordance with the present disclosure are or comprises sequences (e.g., epitopes, fragments, complete proteins) of VZV proteins found in the VZV tegument.

Among other things, the present disclosure provides an insight that, in some embodiments, it may be desirable to include two or more different epitopes, optionally from two or more different VZV proteins, in pharmaceutical composition (e.g., immunogenic composition, e.g., vaccine) compositions, which can be useful in the treatment of VZV.

### Glycoprotein E (gE)

In some embodiments, a provided composition (e.g., a pharmaceutical composition, e.g., immunogenic composition, e.g., vaccine) comprises or delivers a VZV gE protein, or fragment or epitope thereof. In some embodiments, a polyribonucleotide as described herein encodes a polypeptide, where the polypeptide comprises one or more antigens, and where the one or more antigens comprises a VZV gE protein, or fragment or epitope thereof. The term “gE antigen” may be used herein to refer to an antigen that includes at least one gE fragment or epitope (e.g., B cell or T cell epitope).

VZV gE is a 623-amino-acid type I membrane protein encoded by open reading frame 68 (ORF68). VZV gE is the most abundant viral glycoprotein expressed on the viral envelope as well as the surface of VZV-infected cells. Cohen, J., S. Straus, and A. Arvin (ed.), “Varicella-zoster virus replication, pathogenesis and management,” 5th ed. Lippincott-Raven, Philadelphia, PA (2006), which is incorporated herein by reference in its entirety.

VZV gE plays a role in polykaryon formation during VZV infection of epidermal cells. Mo, C., et al., “Glycoprotein E of varicella-zoster virus enhances cell-cell contact in polarized epithelial cells,” J. Virol. 74:11377-11387 (2000); Moffat, J., et al., “Glycoprotein I of varicella-zoster virus is required for viral replication in skin and T cells,” J. Virol. 76:8468-8471 (2002); Moffat, J., et al., “Functions of the C-terminal domain of varicella-zoster virus glycoprotein E in viral replication in vitro and skin and T-cell tropism in vivo,” J. Virol., 78:12406-12415 (2004), each of which is incorporated herein by reference in its entirety. VZV gE has also been suggested to be involved in the retrograde and anterograde axonal transport of virions during primary infection and reactivation from latency. Dingwell, K. S., et al., “Glycoproteins E and I facilitate neuron-to-neuron spread of herpes simplex virus,” J. Virol. 69:7087-7098 (1995); Enquist, L. W., et al., “Directional spread of an alpha-herpesvirus in the nervous system,” Vet. Microbiol. 86:5-16 (2002); Polcicova, K., et al., “The extracellular domain of herpes simplex virus gE is indispensable for efficient cell-to-cell spread: evidence for gE/gI receptors,” J. Virol. 79:11990-12001 (2005); Tirabassi, R. S., et al., “Role of the pseudorabies virus gI cytoplasmic domain in neuroinvasion, virulence, and posttranslational N-linked glycosylation,” J. Virol. 74:3505-3516 (2000); Wang, F., et al., “Herpes simplex virus type 1 glycoprotein E is required for axonal localization of capsid, tegument, and membrane glycoproteins,” J. Virol. 79:13362-13372 (2005), each of which is incorporated herein by reference in its entirety. VZV gE has also demonstrated functional features more commonly observed in non-viral cell surface receptors. For example, gE expressed on the surface of infected cells has the ability to function as an Fc receptor and interact with non-specific IgG, a function which could be of immunological significance. Furthermore, VZV gE has been demonstrated to function as a low-density lipo-protein receptor. “Varicella-Zoster Glycoprotein gE: Endocytosis and Trafficking of the Fc Receptor,” J. Infect. Dis. 10.1086 (1998); Olson, J. K., et al.,

VZV gE has similarities to, e.g., orthologous gE proteins in other human alphaherpesviruses with the important exception that gE is essential for VZV replication. Cohen (2006); Mo, C., et al., “Glycoprotein E of varicella-zoster virus enhances cell-cell contact in polarized epithelial cells,” J. Virol. 74:11377-11387 (2000), each of which is incorporated herein by reference in its entirety. However, VZV gE contains a large N-terminal region that is not conserved in other alphaherpesviruses. Berarducci, B., M. et al., “Essential functions of the unique N-terminal region of the varicella-zoster virus glycoprotein E ectodomain in viral replication and in the pathogenesis of skin infection,”. J. Virol., 80:9481-9496 (2006), which is incorporated herein by reference in its entirety. Mutational analysis of the unique gE N-terminal region identified critical subdomains for VZV skin and T-cell tropism. Berarducci (2006), which is incorporated herein by reference in its entirety. Amino acids (aa) 51 to 187 are essential for viral replication in skin and T-cell xenografts. A partial deletion removing residues 28 to 90 in the gEΔ27-90 mutant abolished the gE interaction with cellular insulin-degrading enzyme (IDE), which is a putative VZV receptor. Berarducci, B., et al., “Functions of the unique N-terminal region of glycoprotein E in the pathogenesis of varicella-zoster virus infection. Proc. Natl. Acad. Sci. U.S.A. 107:282-287 (2010). Despite an inability to bind IDE, gEΔ27-90 was able to replicate in skin, but with decreased cell-cell spread, and in T-cell xenografts. The ectodomain of VZV gE contains two cysteine-rich regions; the deletion of the first cysteine region in the gEΔCys mutant abolished the VZV gE and gI interaction and severely impaired cell-cell spread, viral entry, and replication in skin xenografts. The small gE endodomain contains motifs that may potentially function in the tissue-specific tropism of VZV. Such VZV domains include an AYRV motif (aa 568 to 571) that mediates gE trafficking to the trans-Golgi network (TGN) and an acidic cluster, SSTT (aa 588 to 601), that is phosphorylated by the VZV ORF47 protein kinase. Kenyon, T. K., et al., “Phosphorylation by the varicella-zoster virus ORF47 protein serine kinase determines whether endocytosed viral gE traffics to the trans-Golgi network or recycles to the cell membrane,” J. Virol., 76:10980-10993 (2002); Moffat, J., et al., “Functions of the C-terminal domain of varicella-zoster virus glycoprotein E in viral replication in vitro and skin and T-cell tropism in vivo,” J. Virol., 78:12406-12415 (2004), each of which is incorporated herein by reference in its entirety. The AYRV motif is of interest in that it was demonstrated to be dispensable in vitro, but mutation reduced skin virulence and, to a lesser extent, replication in T cells in vivo. Mutation of the SSTT phosphorylation motif had no effect on replication in vitro or in skin and T-cell xenografts in vivo. Moffatt (2004), which is incorporated herein by reference in its entirety.

In some embodiments, a provided pharmaceutical composition (e.g., immunogenic composition, e.g., VZV vaccine) comprises or delivers an antigen that is or comprises a N-terminal region of a VZV gE protein. In some embodiments, a provided pharmaceutical composition (e.g., immunogenic composition, e.g., VZV vaccine) comprises or delivers an antigen that is or comprises amino acids 51 to 187 of a VZV gE protein. In some embodiments, a provided pharmaceutical composition (e.g., immunogenic composition, e.g., VZV vaccine) comprises or delivers an antigen that is or comprises amino acids 28 to 90 of a VZV gE protein. In some embodiments, a provided pharmaceutical composition (e.g., immunogenic composition, e.g., VZV vaccine) comprises or delivers an antigen that is or comprises amino acids 568 to 571 of a VZV gE protein. In some embodiments, a provided pharmaceutical composition (e.g., immunogenic composition, e.g., VZV vaccine) comprises or delivers an antigen that is or comprises amino acids 588 to 601) of a VZV gE protein.

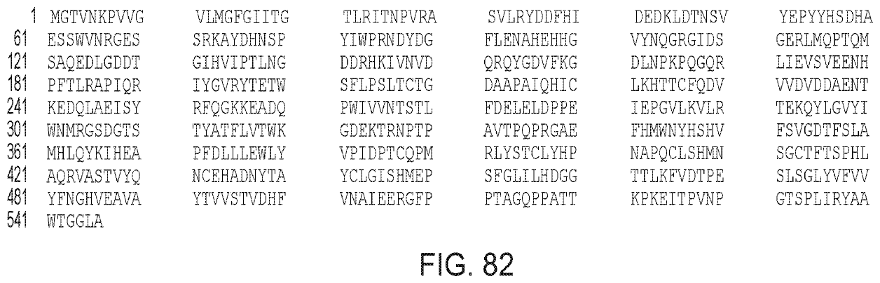

In some embodiments, a provided pharmaceutical composition (e.g., immunogenic composition, e.g., VZV vaccine) comprises or delivers an antigen that is or comprises a sequence as shown in FIG. 82. In some embodiments, a provided pharmaceutical composition (e.g., immunogenic composition, e.g., VZV vaccine) comprises or delivers an antigen that found within a sequence as shown in FIG. 82.

### Glycoprotein B (gb)

In some embodiments, a provided composition (e.g., a pharmaceutical composition, e.g., immunogenic composition, e.g., vaccine) comprises or delivers a VZV gB protein, or fragment or epitope thereof. In some embodiments, a polyribonucleotide as described herein encodes a polypeptide, where the polypeptide comprises one or more antigens, and where the one or more antigens comprises a VZV gB protein, or fragment or epitope thereof. VZV gB binds directly to myelin associated glycoprotein (MAG; Siglec4) to facilitate attachment and invasion. gB-MAG interaction is likely not the sole interaction to facilitate attachment and invasion, as cells lacking MAG are still susceptible to VZV infection. After invasion, gB production continues to occur in the host ER, where mature gB are trafficked to the trans golgi, and similar to other glycoproteins, ultimately incorporated into nascent virus particles: uniquely, a portion of gB is trafficked to the nuclear envelope to potentially play a role in egress of the genome containing viral capsid from this space. GB is highly conserved throughout the Herpesvidae family, giving credence to the notion that this glycoprotein constitutes a core component of VZV fusion and entry into the host.

### Glycoprotein H (gH)

In some embodiments, a provided composition (e.g., a pharmaceutical composition, e.g., immunogenic composition, e.g., vaccine) comprises or delivers a VZV gH protein, or fragment or epitope thereof. In some embodiments, a polyribonucleotide as described herein encodes a polypeptide, where the polypeptide comprises one or more antigens, and where the one or more antigens comprises a VZV gH protein, or fragment or epitope thereof. In some embodiments, a provided pharmaceutical composition (e.g., immunogenic composition, e.g., vaccine) comprises or delivers a VZV gH protein, or fragment or epitope thereof. VZV gH have been demonstrated to play an important role in membrane fusion. Antibodies targeting the N-terminus of gH have been demonstrated to inhibit fusion, indicating the importance of gH in this process. In addition to its importance in membrane fusion, gh has also been shown to be required for viral replication. Mutations in the gene encoding for gH have been shown to produce severe replication defects in human skin xenographs, but have little effect in cell-culture, implicating gH in defining certain aspects of VZV tropism.

Glycoprotein H-Glycoprotein L (gH-gL)

In some embodiments, a provided composition (e.g., a pharmaceutical composition, e.g., immunogenic composition, e.g., vaccine) comprises or delivers a VZV gH protein, or fragment or epitope thereof. In some embodiments, a provided pharmaceutical composition (e.g., immunogenic composition, e.g., vaccine) comprises or delivers a VZV gL protein, or fragment or epitope thereof. In some embodiments, a polyribonucleotide as described herein encodes a polypeptide, where the polypeptide comprises one or more antigens, and where the one or more antigens comprises a VZV gH protein, or fragment or epitope thereof. In some embodiments, a polyribonucleotide as described herein encodes a polypeptide, where the polypeptide comprises one or more antigens, and where the one or more antigens comprises a VZV gL protein, or fragment or epitope thereof.

VZV gH and gL have been shown to form a stable heterodimer and in this complex, perform a similar function as gB, aiding in virion attachment and invasion. Monoclonal antibodies specifically targeting gH have been shown to be sufficient in neutralizing the fusion event that the gH-gL complex usually facilitates. Similar to gB, the gH-gL complex is highly conserved in the Herpesviridae family and is similarly thought to constitute a core component of VZV fusion and entry into the host.

### Glycoprotein C (gC)

In some embodiments, a provided composition (e.g., a pharmaceutical composition, e.g., immunogenic composition, e.g., vaccine) comprises or delivers a VZV gC protein, or fragment or epitope thereof. In some embodiments, a polyribonucleotide as described herein encodes a polypeptide, where the polypeptide comprises one or more antigens, and where the one or more antigens comprises a VZV gC protein, or fragment or epitope thereof. VZV gC is unique relative to its other VZV glycoprotein counterparts in that it is expressed and accumulates in the very late stages of VZV infection, and is thus considered a true late stage protein. Although gC has been shown to bind to heparin sulfate in other Herpesviridae viruses, this interaction has never been proven in VZV. Consequently, the neutralizing potential of antibodies which bind gC are not entirely clear, making gC a more ambiguous therapeutic target.

Glycoprotein M-Glycoprotein N (gM-gN)

In some embodiments, a provided composition (e.g., a pharmaceutical composition, e.g., immunogenic composition, e.g., vaccine) comprises or delivers a VZV gM protein, or fragment or epitope thereof. In some embodiments, a provided pharmaceutical composition (e.g., immunogenic composition, e.g., vaccine) comprises or delivers a VZV gN protein, or fragment or epitope thereof. In some embodiments, a polyribonucleotide as described herein encodes a polypeptide, where the polypeptide comprises one or more antigens, and where the one or more antigens comprises a VZV gM protein, or fragment or epitope thereof. In some embodiments, a polyribonucleotide as described herein encodes a polypeptide, where the polypeptide comprises one or more antigens, and where the one or more antigens comprises a VZV gN protein, or fragment or epitope thereof.

gM and gN form a heterodimer that perform an ambiguous function; however, it has been shown that the disruption of expression of these genes leads to reduced in-vitro virulence, which would indicate importance in the VZV life cycle via unknown mechanisms.

### ORF66

In some embodiments, a provided composition (e.g., a pharmaceutical composition, e.g., immunogenic composition, e.g., vaccine) comprises or delivers a VZV US3 kinase protein, or fragment or epitope thereof. In some embodiments, a polyribonucleotide as described herein encodes a polypeptide, where the polypeptide comprises one or more antigens, and where the one or more antigens comprises a VZV U3 kinase protein, or fragment or epitope thereof. VZV ORF66 is found in all neurotropic alpha herpesviruses and produces a protein often referred to as US3 kinase. VZV ORF66 encodes a serine/threonine protein kinase and described to be important in immune evasion during the more vulnerable stage of reactivation from latency to acute infection where virion number is low and rapid immune responses have the potential for clearing the virus. It has been shown that the product of ORF66 is capable of inhibiting the expression of MHC class I on the surface of the cells it infects in a kinase dependent manner by disrupting the transport of MHC class I out of the Golgi, causing its sequestration in that organelle. Although this process is kinase dependent, the direct phosphorylation of MHC class I b has never been shown. ORF66 mediated sequestration of MHC class I in the Golgi has been hypothesize as an important mechanism in the establishment and maintenance of VZV latent stage infection. Considering the tropism of VZV in cells that are not professional antigen presenting cells and thus only capable of presenting to CD8 T-cells via MHC class I, the protein produced by ORF66 may disarm one of the most universally potent pathways of immune activation and is certainly a worthy target for therapeutic intervention.

### Intermediate Early 4 (IE4)

In some embodiments, a provided composition (e.g., a pharmaceutical composition, e.g., immunogenic composition, e.g., vaccine) comprises or delivers a VZV IE4 protein, or fragment or epitope thereof. In some embodiments, a polyribonucleotide as described herein encodes a polypeptide, where the polypeptide comprises one or more antigens, and where the one or more antigens comprises a VZV IE4 protein, or fragment or epitope thereof. VZV IE4 encoded by ORF4 is an immediate early protein within the tegument and one of 6 proteins expressed during latent infection or the early stages of reactivation. ORF4 encodes a 51 kDA phosphoprotein present in the Tegument and ultimately in the nucleus early in infection. ORF4 has been shown in mouse models to be important for latent stage infection, but not essential to it. Although the mechanism behind its importance is unclear, ORF4 has been shown to interact and stabilize ORF6 which may contribute to its physiological importance.

### ORF47

In some embodiments, a provided composition (e.g., a pharmaceutical composition, e.g., immunogenic composition, e.g., vaccine) comprises or delivers a protein encoded by ORF47, or fragment or epitope thereof. In some embodiments, a polyribonucleotide as described herein encodes a polypeptide, where the polypeptide comprises one or more antigens, and where the one or more antigens comprises a protein encoded by ORF47, or fragment or epitope thereof. VZV ORF47 expression produces one of as little as 6 proteins shown to be expressed during latency or early activation. ORF47 is a protein kinase that phosphorylates important glycoproteins gI and gE. ORF47 protein kinase has been shown to be expressed at levels similar to other VZZ proteins which are hallmarks of VZV latent infection; however, rodent models have shown this protein not be essential to the establishment of latent infection. Although dispensable for latent infection, its phosphorylation of important acute stage proteins may make it important to the pathogenesis of VZV. Furthermore, pre-existing cellular immunity formed against antigens that exist in abundance during the acute stage of infection and that become scarce during the latent stage/early activation can provide the basis for utilizing a non-essential protein like ORF47 protein kinase in a vaccine strategy. Targeting immune response towards ORF47 protein kinase may provide a valuable mechanism to activate the immune response towards what is typically an invisible latent stage infection and in turn prime pre-existing memory responses against acute stage proteins re-emerging during reactivation while viremia is still low.

### Intermediate Early 63 (IE63)

In some embodiments, a provided composition (e.g., a pharmaceutical composition, e.g., immunogenic composition, e.g., vaccine) comprises or delivers a VZV IE63 protein, or fragment or epitope thereof. In some embodiments, a polyribonucleotide as described herein encodes a polypeptide, where the polypeptide comprises one or more antigens, and where the one or more antigens comprises a VZV IE63 protein, or fragment or epitope thereof. VZV IE63, encoded by ORF63 and ORF70, is an immediate-early protein present in virions and essential for the establishment of latent infection and is the most highly expressed latent stage protein. Infecting rodent models with a mutated copy of ORF63 results in a dramatic reduction in latent stage persistence of VZV. IE63's important in latency has immense immunological implications, as it is believed to be involved in the repression of VZV thymidine kinase and DNA polymerase promotors and induce global repression of gene expression and protein production, a hallmark feature of VZV latency. It is this quiescence which defines the difficulty in mounting an immune response capable of clearing VZV, as the antigens such immune responses are directed towards are no longer expressed during the latent stage. Therefore, targeting this protein could provide a twofold benefit, firstly provide an essential target to prevent VZV persistence and reactivation which could potentially independently lead to the clearance of this stage of the virus, and secondly, provide a mechanism whereby VZV can no longer remain hidden from innate immune surveillance mechanisms and memory immune responses.

### Intermediate Early 62 (IE62)

In some embodiments, a provided composition (e.g., a pharmaceutical composition, e.g., immunogenic composition, e.g., vaccine) comprises or delivers a VZV IE62 protein, or fragment or epitope thereof. In some embodiments, a polyribonucleotide as described herein encodes a polypeptide, where the polypeptide comprises one or more antigens, and where the one or more antigens comprises a VZV IE62 protein, or fragment or epitope thereof. IE62 is an immediate early protein within the tegument encoded by ORF62 and ORF71. IE62 has been identified as a structural component of VZV virions located within the tegument. IE62 is capable of modulating host innate immune signaling by antagonizing activation of interferon response factor 3, complimenting the immune modulating activity of IE63 and adding to the repertoire of host immune modulating proteins that likely lead to latent VZV infection. IE62 has been shown to play an important role in VZV skin pathogenesis. IE62 localization is dynamic during the progression of VZV from lytic to latent infection. During the lytic stage of infection, IE62 initially localizes to the nucleus of infected cells and likely plays a role in gene transactivation and VZV replication; however, as progression to latent infection occurs, IE62 is progressively sequestered from the nucleus and exhibits a cytoplasmic localization. This change in localization prevents IE62 of performing its lytic stage function of VZV gene transactivation and replication and likely contributes to the hallmark feature of latent infection, reduced viral replication and protein expression.

### CMV Protein Sequences

In some embodiments, a provided composition (e.g., a pharmaceutical composition, e.g., immunogenic composition, e.g., vaccine) comprises or delivers (e.g., causes expression of in a recipient organism, for example by administration of a nucleic acid construct, such as an RNA construct as described herein, that encodes it) an antigen that is or comprises one or more epitopes (e.g., one or more B-cell and/or one or more T-cell epitopes) of a CMV protein. In some embodiments, a composition described herein induces a relevant immune response effective against CMV (e.g., by targeting a CMV protein).

In some embodiments, a provided composition (e.g., a pharmaceutical composition, e.g., immunogenic composition, e.g., vaccine) comprises or delivers an antigen that is or comprises a full-length CMV protein. In some embodiments, a provided composition (e.g., a pharmaceutical composition, e.g., immunogenic composition, e.g., vaccine) comprises or delivers an antigen that is or comprises a portion of a CMV protein that is less than a full-length CMV protein. In some embodiments, a provided composition (e.g., a pharmaceutical composition, e.g., immunogenic composition, e.g., vaccine) comprises or delivers a chimeric polypeptide that is or comprises part or all of a CMV protein and one or more heterologous polypeptide elements.

In some embodiments, an antigen that is included in and/or delivered by a provided pharmaceutical composition (e.g., immunogenic composition, e.g., CMV vaccine) is or comprises one or more peptide fragments of a CMV antigen; in some such embodiments, each of the one or more peptide fragments includes at least one epitope (e.g., one or more B cell epitopes and/or one or more T cell epitopes), for example as may be predicted, selected, assessed and/or characterized as described herein.

In some embodiments, an antigen that is included in and/or delivered by a provided pharmaceutical composition (e.g., immunogenic composition, e.g., CMV vaccine) is or comprises a plurality of peptide fragments of one or more CMV antigens. In some embodiments, a single polypeptide antigen may include a plurality of such fragments, e.g., presented as a string antigen as described herein.

In some embodiments, one or more CMV epitopes (e.g., included in part or all of a CMV protein) may be linked with one or more sequences with which it is linked in nature; in some such embodiments, such sequence(s) may be or comprise one or more heterologous elements (e.g., one or more elements, not naturally found in the relevant CMV protein or not naturally found directly linked to the relevant CMV epitope(s)). For example, in some embodiments, an antigen peptide provided and/or utilized in accordance with the present disclosure may include one or more linker elements and/or one or more membrane association elements and/or one or more secretion elements, etc. In some embodiments, an antigen peptide may comprise a plurality of CMV protein fragments or epitopes separated from one another by linkers.

In some embodiments, a CMV protein, or fragment or epitope thereof, utilized in an antigen as described herein may include one or more sequence alterations relative to a particular reference CMV protein, or fragment or epitope thereof. For example, in some embodiments, a utilized antigen may include one or more sequence variations found in circulating strains or predicted to arise, e.g., in light of assessments of sequence conservation and/or evolution of CMV proteins over time and/or across strains. Alternatively or additionally, in some embodiments, a utilized antigen may include one or more sequence variations selected, for example, to impact stability, folding, processing and/or display of the antigen or any epitope thereof.

In some embodiments, a CMV protein, or fragment or epitope thereof, utilized in an antigen as described herein shows at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with a relevant corresponding reference (e.g., wild type) protein, fragment or epitope. In some embodiments, a CMV protein, or fragment or epitope thereof, utilized in an antigen as described herein shows at least 80%, 81%, 82%, 83%, 84%, 85%, 86%, 87%, 88%, 89%, 90%, 91%, 92%, 93%, 94%, 95%, 96%, 97%, 98%, 99%, or 100% sequence homology (i.e., identity or conservative substitution as is understood in the art) amino acid sequence identity with a relevant corresponding reference (e.g., wild type) protein, fragment or epitope. Moreover, in some embodiments, a CMV protein, or fragment or epitope thereof, utilized in an antigen as described herein shares conserved amino acid residues (e.g., at corresponding positions) with a relevant corresponding reference (e.g., wild type) protein, fragment or epitope. Those skilled in the art will appreciate that, in general, lower percent identity or homology may be tolerated for shorter peptides, as a single change will by definition have a larger impact on percent identity or homology when considered relative to a smaller number of residues. For example, those skilled in the art will appreciate that, for sequences longer than about 20 amino acids, percent identity or homology is typically greater than about 80%; for sequences longer than about 50 amino acids, percent identity or homology is typically greater than about 90%.

In some embodiments, assessments of degree of conservation may consider the physiochemical difference between two amino acids as described, for example, in WO2014/180569, which is incorporated herein by reference in its entirety. It is well known in molecular evolution that amino acids that interchange frequently are likely to have chemical and physical similarities whereas amino acids that interchange rarely are likely to have different physico-chemical properties. The likelihood for a given substitution to occur in nature compared with the likelihood for this substitution to occur by chance can measured by log-odds matrices. The patterns observed in log-odds matrices imposed by natural selection “reflect the similarity of the functions of the amino acid residues in their weak interactions with one another in the three dimensional conformation of proteins” (see Dayhoff et al. Atlas of protein sequence and structure 5:345, 1978180569, which is incorporated herein by reference in its entirety). In some embodiments, evolutionary based log-odds matrices, which may be referred to as “T scores” can be used to reflect extent to which a sequence variation might impact T cell recognition. Substitutions with positive T scores (i.e., log-odds) are likely to occur in nature, and hence correspond to two amino acids that have similar physico-chemical properties. Substitutions with positive T scores would have a lower likelihood of altering immunogenicity. Conversely, substitutions with negative T scores reflect substitutions that are unlikely to occur in nature and hence correspond to two amino acids that have significantly different physico-chemical properties. Such substitutions would have a greater chance of altering immunogenicity. In some embodiments, presence of negative T score substitutions within a sequence, even if it is otherwise highly conserved, may indicate that it would be relatively less useful in a vaccine antigen as described herein.

In some embodiments, a utilized antigen induces an immune response that targets a CMV envelope glycoprotein. In some embodiments, one or more antigens induce an immune response that targets a CMV envelope glycoprotein. In some embodiments, one or more antigens comprises one or more CMV protein sequences (e.g., conserved sequences and/or sequences that are or comprise one or more B cell epitopes and/or one or more CD4 epitopes and/or one or more CD8 epitopes) of an antigen or epitope of a CMV envelope glycoprotein. In some embodiments, one or more antigens is or comprises a CMV gM protein or a fragment or epitope thereof. In some embodiments, one or more antigens is or comprises a CMV gL protein or a fragment or epitope thereof. In some embodiments, one or more antigens is or comprises a CMV gB protein or a fragment or epitope thereof. In some embodiments, one or more antigens is or comprises a CMV gN protein or a fragment or epitope thereof. In some embodiments, one or more antigens is or comprises a CMV gO protein or a fragment or epitope thereof. In some embodiments, one or more antigens is or comprises a CMV g24 protein or a fragment or epitope thereof. In some embodiments, one or more antigens is or comprises a CMV gH protein or a fragment or epitope thereof. In some embodiments, one or more antigens is or comprises a RL10 protein, fragment, or epitope thereof. In some embodiments, one or more antigens is or comprises a UL131A protein, fragment, or epitope thereof. In some embodiments, one or more antigens is or comprises a UL132 protein, fragment, or epitope thereof. In some embodiments, one or more antigens is or comprises a UL33 protein, fragment, or epitope thereof. In some embodiments, one or more antigens is or comprises a UL37 protein, fragment, or epitope thereof. In some embodiments, one or more antigens is or comprises a UL4 protein, fragment, or epitope thereof. In some embodiments, one or more antigens is or comprises a UL40 protein, fragment, or epitope thereof. In some embodiments, one or more antigens is or comprises a UL78 protein, fragment, or epitope thereof. In some embodiments, one or more antigens is or comprises a US27 protein, fragment, or epitope thereof. In some embodiments, one or more antigens is or comprises a US28 protein, fragment, or epitope thereof.

In some embodiments, one or more antigens is or comprises one or more of a CMV gM protein, fragment or epitope thereof, a CMV gL protein, fragment or epitope thereof, a CMV gC protein, fragment or epitope thereof, a CMV gB protein, fragment or epitope thereof, a CMV gN protein, fragment or epitope thereof, a CMV gO protein, fragment or epitope thereof, a CMV g24 protein, fragment or epitope thereof, a CMV gH protein, fragment or epitope thereof, or a combination thereof. In some embodiments, one or more antigens is or comprises one or more of a CMV gM protein, fragment or epitope thereof, a CMV gL protein, fragment or epitope thereof, a CMV gC protein, fragment or epitope thereof, a CMV gB protein, fragment or epitope thereof, a CMV gN protein, fragment or epitope thereof, a CMV gO protein, fragment or epitope thereof, a CMV g24 protein, fragment or epitope thereof, a CMV gH protein, fragment or epitope thereof, a RL10 protein, fragment, or epitope thereof, a UL131A protein, fragment, or epitope thereof, a UL132 protein, fragment, or epitope thereof, a UL33 protein, fragment, or epitope thereof, a UL37 protein, fragment, or epitope thereof, a UL4 protein, fragment, or epitope thereof, a UL40 protein, fragment, or epitope thereof, a UL78 protein, fragment, or epitope thereof, a US27 protein, fragment, or epitope thereof, a US28 protein, fragment, or epitope thereof, or a combination thereof.

In some embodiments, a utilized antigen induces an immune response that targets a CMV tegument protein. In some embodiments, one or more antigens is or comprises a CMV TRS1 protein, fragment or epitope thereof. In some embodiments, one or more antigens is or comprises a CMV UL7 protein, fragment or epitope thereof. In some embodiments, one or more antigens is or comprises a CMV UL23 protein, fragment or epitope thereof. In some embodiments, one or more antigens is or comprises a CMV UL24 protein, fragment or epitope thereof. In some embodiments, one or more antigens is or comprises a CMV UL25 protein, fragment or epitope thereof. In some embodiments, one or more antigens is or comprises a CMV UL26 protein, fragment or epitope thereof. In some embodiments, one or more antigens is or comprises a CMV pp150 protein, fragment or epitope thereof. In some embodiments, one or more antigens is or comprises a CMV UL35 protein, fragment or epitope thereof. In some embodiments, one or more antigens is or comprises a CMV vICA protein, fragment or epitope thereof. In some embodiments, one or more antigens is or comprises a CMV UL43 protein, fragment or epitope thereof. In some embodiments, one or more antigens is or comprises a CMV UL37 protein, fragment or epitope thereof. In some embodiments, one or more antigens is or comprises a CMV large tegument protein, fragment or epitope thereof. In some embodiments, one or more antigens is or comprises a CMV UL51 protein, fragment or epitope thereof. In some embodiments, one or more antigens is or comprises a CMV pp71 protein, fragment or epitope thereof. In some embodiments, one or more antigens is or comprises a CMV pp65 protein, fragment or epitope thereof. In some embodiments, one or more antigens is or comprises a CMV UL88 protein, fragment or epitope thereof. In some embodiments, one or more antigens is or comprises a CMV UL16 protein, fragment or epitope thereof. In some embodiments, one or more antigens is or comprises a CMV UL14 protein, fragment or epitope thereof. In some embodiments, one or more antigens is or comprises a CMV US22 protein, fragment or epitope thereof. In some embodiments, one or more antigens is or comprises a CMV US24 protein, fragment or epitope thereof.

In some embodiments, one or more antigens is or comprises a CMV TRS1 protein, fragment or epitope thereof, a CMV UL7 protein, fragment or epitope thereof, a CMV UL23 protein, fragment or epitope thereof, a CMV UL24 protein, fragment or epitope thereof, a CMV UL25 protein, fragment or epitope thereof, a CMV UL26 protein, fragment or epitope thereof, a CMV pp150 protein, fragment or epitope thereof, a CMV UL35 protein, fragment or epitope thereof, a CMV vICA protein, fragment or epitope thereof, a CMV UL43 protein, fragment or epitope thereof, a CMV UL37 protein, fragment or epitope thereof, a CMV large tegument protein, fragment or epitope thereof, a CMV UL51 protein, fragment or epitope thereof, a CMV pp71 protein, fragment or epitope thereof, a CMV pp65 protein, fragment or epitope thereof, a CMV UL88 protein, fragment or epitope thereof, a CMV UL16 protein, fragment or epitope thereof, a CMV UL14 protein, fragment or epitope thereof, a CMV US22 protein, fragment or epitope thereof, a CMV US24 protein, fragment or epitope thereof, or a combination thereof.

In some embodiments, a utilized antigen induces an immune response that targets a CMV membrane protein. In some embodiments, one or more antigens is or comprises a CMV RL11 protein, fragment or epitope thereof. In some embodiments, one or more antigens is or comprises a CMV RL12 protein, fragment or epitope thereof. In some embodiments, one or more antigens is or comprises a CMV RL13 protein, fragment or epitope thereof. In some embodiments, one or more antigens is or comprises a CMV UL1 protein, fragment or epitope thereof. In some embodiments, one or more antigens is or comprises a CMV UL10 protein, fragment or epitope thereof. In some embodiments, one or more antigens is or comprises a CMV UL11 protein, fragment or epitope thereof. In some embodiments, one or more antigens is or comprises a CMV UL119 protein, fragment or epitope thereof. In some embodiments, one or more antigens is or comprises a CMV UL120 protein, fragment or epitope thereof. In some embodiments, one or more antigens is or comprises a CMV UL121 protein, fragment or epitope thereof. In some embodiments, one or more antigens is or comprises a CMV UL124 protein, fragment or epitope thereof. In some embodiments, one or more antigens is or comprises a CMV UL139 protein, fragment or epitope thereof. In some embodiments, one or more antigens is or comprises a CMV UL14, fragment or epitope thereof. In some embodiments, one or more antigens is or comprises a CMV UL141 protein, fragment or epitope thereof. In some embodiments, one or more antigens is or comprises a CMV UL142 protein, fragment or epitope thereof. In some embodiments, one or more antigens is or comprises a CMV UL144 protein, fragment or epitope thereof. In some embodiments, one or more antigens is or comprises a CMV UL147 protein, fragment or epitope thereof. In some embodiments, one or more antigens is or comprises a CMV UL148 protein, fragment or epitope thereof. In some embodiments, one or more antigens is or comprises a CMV UL16 protein, fragment or epitope thereof. In some embodiments, one or more antigens is or comprises a CMV UL18 protein, fragment or epitope thereof. In some embodiments, one or more antigens is or comprises a CMV UL20 protein, fragment or epitope thereof. In some embodiments, one or more antigens is or comprises a CMV UL6 protein, fragment or epitope thereof. In some embodiments, one or more antigens is or comprises a CMV UL7 protein, fragment or epitope thereof. In some embodiments, one or more antigens is or comprises a CMV UL8 protein, fragment or epitope thereof. In some embodiments, one or more antigens is or comprises a CMV UL9 protein, fragment or epitope thereof. In some embodiments, one or more antigens is or comprises a CMV US10 protein, fragment or epitope thereof. In some embodiments, one or more antigens is or comprises a CMV US11 protein, fragment or epitope thereof. In some embodiments, one or more antigens is or comprises a CMV US12 protein, fragment or epitope thereof. In some embodiments, one or more antigens is or comprises a CMV US13 protein, fragment or epitope thereof. In some embodiments, one or more antigens is or comprises a CMV US14 protein, fragment or epitope thereof. In some embodiments, one or more antigens is or comprises a CMV US15 protein, fragment or epitope thereof. In some embodiments, one or more antigens is or comprises a CMV US16 protein, fragment or epitope thereof. In some embodiments, one or more antigens is or comprises a CMV US17 protein, fragment or epitope thereof. In some embodiments, one or more antigens is or comprises a CMV US18 protein, fragment or epitope thereof. In some embodiments, one or more antigens is or comprises a CMV US19 protein, fragment or epitope thereof. In some embodiments, one or more antigens is or comprises a CMV US20 protein, fragment or epitope thereof. In some embodiments, one or more antigens is or comprises a CMV US21 protein, fragment or epitope thereof. In some embodiments, one or more antigens is or comprises a CMV US29 protein, fragment or epitope thereof. In some embodiments, one or more antigens is or comprises a CMV US3 protein, fragment or epitope thereof. In some embodiments, one or more antigens is or comprises a CMV US30 protein, fragment or epitope thereof. In some embodiments, one or more antigens is or comprises a CMV US6 protein, fragment or epitope thereof. In some embodiments, one or more antigens is or comprises a CMV US7 protein, fragment or epitope thereof. In some embodiments, one or more antigens is or comprises a CMV US8 protein, fragment or epitope thereof. In some embodiments, one or more antigens is or comprises a CMV US9 protein, fragment or epitope thereof.

In some embodiments, one or more antigens is or comprises a CMV RL11 protein, fragment or epitope thereof, a CMV RL12 protein, fragment or epitope thereof, a CMV RL13 protein, fragment or epitope thereof, a CMV UL1 protein, fragment or epitope thereof, a CMV UL10 protein, fragment or epitope thereof, a CMV UL11 protein, fragment or epitope thereof, a CMV UL119 protein, fragment or epitope thereof, a CMV UL120 protein, fragment or epitope thereof, a CMV UL121 protein, fragment or epitope thereof, a CMV UL124 protein, fragment or epitope thereof, a CMV UL139 protein, fragment or epitope thereof, a CMV UL14, fragment or epitope thereof, a CMV UL141 protein, fragment or epitope thereof, a CMV UL142 protein, fragment or epitope thereof, a CMV UL144 protein, fragment or epitope thereof, a CMV UL147 protein, fragment or epitope thereof, a CMV UL148 protein, fragment or epitope thereof, a CMV UL16 protein, fragment or epitope thereof, a CMV UL18 protein, fragment or epitope thereof, a CMV UL20 protein, fragment or epitope thereof, a CMV UL6 protein, fragment or epitope thereof, a CMV UL7 protein, fragment or epitope thereof, a CMV UL8 protein, fragment or epitope thereof, a CMV UL9 protein, fragment or epitope thereof, a CMV US10 protein, fragment or epitope thereof, a CMV US11 protein, fragment or epitope thereof, a CMV US12 protein, fragment or epitope thereof, a CMV US13 protein, fragment or epitope thereof, a CMV US14 protein, fragment or epitope thereof, a CMV US15 protein, fragment or epitope thereof, a CMV US16 protein, fragment or epitope thereof, a CMV US17 protein, fragment or epitope thereof, a CMV US18 protein, fragment or epitope thereof, a CMV US19 protein, fragment or epitope thereof, a CMV US20 protein, fragment or epitope thereof, a CMV US21 protein, fragment or epitope thereof, a CMV US29 protein, fragment or epitope thereof, a CMV US3 protein, fragment or epitope thereof, a CMV US30 protein, fragment or epitope thereof, a CMV US6 protein, fragment or epitope thereof, a CMV US7 protein, fragment or epitope thereof, a CMV US8 protein, fragment or epitope thereof, a CMV US9 protein, fragment or epitope thereof, or a combination thereof.

In some embodiments, one or more antigens is or comprises one or more of a CMV TRS1 protein, fragment or epitope thereof, a CMV UL7 protein, fragment or epitope thereof, a CMV UL23 protein, fragment or epitope thereof, a CMV UL24 protein, fragment or epitope thereof, a CMV UL25 protein, fragment or epitope thereof, a CMV UL26 protein, fragment or epitope thereof, a CMV pp150 protein, fragment or epitope thereof, a CMV UL35 protein, fragment or epitope thereof, a CMV vICA protein, fragment or epitope thereof, a CMV UL43 protein, fragment or epitope thereof, a CMV UL37 protein, fragment or epitope thereof, a CMV large tegument protein, fragment or epitope thereof, a CMV UL51 protein, fragment or epitope thereof, a CMV pp71 protein, fragment or epitope thereof, a CMV pp65 protein, fragment or epitope thereof, a CMV UL88 protein, fragment or epitope thereof, a CMV UL16 protein, fragment or epitope thereof, a CMV UL14 protein, fragment or epitope thereof, a CMV US22 protein, fragment or epitope thereof, a CMV US24 protein, fragment or epitope thereof, or a combination thereof.

In some embodiments, one or more antigens is or comprises one or more antigens listed in Table 8A, Table 8B, and/or Table 9A herein. In some embodiments, one or more antigens is or comprises one or more antigens listed in Table 9A herein. In some embodiments, one or more antigens is or comprises one or more antigens listed in Table 9B herein. In some embodiments, one or more antigens is or comprises one or more antigens encoded by respective genes listed in Tables 6A-6F and/or Tables 7A-7B.

In some embodiments, one or more antigens in accordance with the present disclosure is or comprises a polypeptide or portion thereof encoded by all or part of TRS1, UL32, UL36, UL44, UL55, UL57, UL75, UL83, UL84, UL86, UL98, UL122, UL123, or a combination thereof.

In some embodiments, an antigen utilized in accordance with the present disclosure includes CMV protein sequences identified and/or characterized by one or more of:
HLA-I or HLA-II binding (e.g., to HLA allele(s) present in a relevant population)
HLA ligandomics data, optionally confirmed by mass spectrometry
Relatively high expression
Sequence conservation
Surface exposure
Serum reactivity
Immunogenicity (e.g., presence of one or more B-cell and/or T-cell epitopes; evidence of ability to induce sterile protection in model systems including, e.g., humans, non-human primates, and/or mice).
Absence of sequences that overlap with human proteome

In some embodiments, such characteristics are experimentally or computationally assessed. In some embodiments, such characteristics are assessed by consultation with published reports.

For example, in some embodiments, HLA-I and/or HLA-II binding is experimentally assessed; in some embodiments it is predicted.

In some embodiments, predicted HLA-I or HLA-II binding is assessed using an algorithm such as neonmhc 1 and/or neonmhc2, which predict and/or characterize likelihood of MHC class I and MHC class II binding, respectively. Alternatively or additionally, in some embodiments, an MHC-peptide presentation prediction algorithm or MHC-peptide presentation predictor is or comprises NetMHCpan or NetMHCIIpan. In some embodiments, a hidden markov model approach may be utilized for MHC-peptide presentation prediction and/or characterization. In some embodiments, the peptide prediction model MARIA may be utilized. In some embodiments, NetMHCpan is not utilized to predict or characterize likelihood of MHC binding for peptides as described herein. In some embodiments, the peptide prediction model MARIA may be utilized. In some embodiments, NetMICIIpan is not utilized to predict or characterize likelihood of MHC binding for peptides as described herein. In some embodiments, neither NetMHCpan nor NetMICIIpan is utilized to predict or characterize likelihood of MHC binding for peptides as described herein. In some embodiments, an MHC-peptide presentation prediction algorithm or MHC-peptide presentation predictor is or comprises RECON® (Real-time Epitope Computation for ONcology), which offers high quality MHC-peptide presentation prediction based on expression, processing and binding capabilities. See, for example, Abelin et al., Immunity 21:315, 2017; Abelin et al., Immunity 15:766, 2019.

In some embodiments, HLA binding and/or ligandomics assessments will consider the geographic region of subjects to be immunized. For example, in some embodiments, HLA allelic diversity will be considered. In some embodiments, antigen(s) included in a provided pharmaceutical composition (e.g., immunogenic composition, e.g., vaccine) will be or comprise peptides (e.g., epitopes) expected or determined, when considered together, to bind to a significant percentage (e.g., at least 60%, 65%, 70%, 75%, 80%, 85%, 90%, 95%, or more) of HLA alleles expected or known to be present in a relevant region or population. In some embodiments, antigen(s) included in a provided pharmaceutical composition (e.g., immunogenic composition, e.g., vaccine) will be or comprise peptides expected or determined, when considered together, to bind to the most prevalent (e.g., the 1, 2, 3, 4, 5, 6, 7, 8, 9, or 10 most prevalent, or at least 1, 2, 3, 4, or 5 of the 10 most prevalent, etc.) HLA alleles expected or known to be present in a relevant region or population).

In some embodiments, expression level is experimentally determined (e.g., in a model system or in infected humans). In some embodiments, expression level is a reported level (e.g., in a published or presented report). In some embodiments, expression level is assessed as RNA (e.g., via RNASeq). In some embodiments (and typically preferably), expression levels is assessed as protein.

In some embodiments, sequence conservation is assessed, for example, using publicly available sequence evaluation software (such as, for example, multiple sequence alignment programs MAFFT, Clustal Omega, etc.). In some embodiments, sequence conservation is determined by consultation with published resources (e.g., sequences). In some embodiments, sequence conservation includes consideration of currently or recently detected strains (e.g., in an active outbreak).

In some embodiments, surface exposure is assessed by reference to publicly available database and/or software.

In some embodiments, serum reactivity is assessed by contacting serum samples from infected individuals with polypeptides including sequences of interest (e.g., as may be displayed via, for example, phage display or peptide array, etc; see, for example, Whittemore et al “A General Method to Discover Epitopes from Sera” PlosOne, 2016; https://doi.org/10.1371/journal.pone.0157462). In some embodiments, serum reactivity is assessed by consultation with literature reports and or database data indicating serum-recognized sequences.

In some embodiments, assessment of immunoreactivity and/or of presence of an epitope may be or comprise consultation with the Immune Epitope Database (IEDB) which those skilled in the art will be aware is a freely available resource funded by NIAID that catalogs experimental data on antibody and T cell epitopes (see iedb.org).

In some embodiments, antigen(s) utilized in accordance with the present disclosure are characterized by dendritic cell presentation which, in turn may be indicative of HLA binding and/or of immunogenicity.

In some embodiments, antigen(s) utilized in accordance with the present disclosure are or comprises sequences (e.g., epitopes, fragments, complete proteins) of CMV proteins found in the CMV envelope. In some embodiments, antigen(s) utilized in accordance with the present disclosure are or comprises sequences (e.g., epitopes, fragments, complete proteins) of CMV proteins found in the CMV tegument. In some embodiments, antigen(s) utilized in accordance with the present disclosure are or comprises sequences (e.g., epitopes, fragments, complete proteins) of CMV proteins found in the CMV membrane.

Among other things, the present disclosure provides an insight that, in some embodiments, it may be desirable to include two or more different epitopes, optionally from two or more different CMV proteins, in pharmaceutical composition (e.g., immunogenic composition, e.g., vaccine) compositions, which can be useful in the treatment of CMV.

### Glycoprotein B (gB)

In some embodiments, a provided composition (e.g., a pharmaceutical composition, e.g., immunogenic composition, e.g., vaccine) comprises or delivers a CMV gB protein, or fragment or epitope thereof. In some embodiments, a polyribonucleotide as described herein encodes a polypeptide, where the polypeptide comprises one or more antigens, and where the one or more antigens comprises a CMV gB protein, or fragment or epitope thereof. CMV glycoprotein B (gB; UL55), is an abundant glycoprotein on the virus envelope and the most highly conserved glycoprotein of the human herpesviruses. Gb has homologs throughout the Herpesviridae family, which are conserved and appear to serve essential, universal functions, as well as specific functions unique to each particular herpesvirus. See, e.g., Isaacson, M. K., Compton, T., “Human cytomegalovirus Glycoprotein B is Required for Virus Entry and Cell-to-Cell Spread but Not for Virion Attachment, Assembly, or Egress,” J Virol., 83(8): 3891-3903 (April 2009), which is incorporated herein by reference in its entirety.

For example, gB is understood to play a critical role in the CMV entry process. In particular, it is believed that gB is involved in the initial virion-tethering step and the more stable attachment step, as well as fusion of the virion with the cell membrane. For this function, gB is able to interact with specific integrin heterodimers, and this interaction is understood to enhance CMV entry into cells. It has also been reported that gB interacts with the epidermal growth factor receptor and may use this interaction to mediate virus entry. Evidence supporting an attachment role for gB includes the ability of soluble gB (gBs) to bind heparin, a soluble mimic of HSPGs. Additionally, gBs manifests two-step binding kinetics to cells, in which the protein is initially dissociable with soluble heparin but quickly becomes resistant to removal by heparin washes. This suggests that gB moves from one receptor to another, likely cellular integrins, during the attachment process. Antibodies to HCMV gB, including those to the DLD and to specific integrin heterodimers, are able to efficiently neutralize virus entry at a postattachment stage, suggesting that gB and integrins are involved in the fusion event.

Additionally, CMV gB can acts as a fusion mediator for the virus. gB contains a heptad repeat region, which is predicted to form an alpha-helical coiled coil, commonly found in many class I viral fusion proteins.

Aside from its role in virus entry and fusion, gB may also be required for cell-to-cell spread of the virus. At the final phase of viral entry, the viral and cellular membranes fuse, releasing the tegument proteins and capsid into the cytoplasm. This fusion event may require gB, as well as the glycoprotein complex, gH/gL. During plaque formation, virions may spread directly through cellular contacts or across cellular junctions. Evidence for the role of gB in CMV cell-to-cell spread includes the ability of gB-neutralizing antibodies to prevent plaque formation in infected cells.

gB proteins from a variety of herpesviruses have also been proposed to be necessary to mediate virion assembly and egress from infected cells. Herpesvirus egress is thought to involve an initial envelopment step at the inner nuclear membrane, followed by a de-envelopment step at the outer nuclear membrane and, last, a re-envelopment step at a Golgi apparatus-derived membrane before the virion-containing vesicle fuses with the plasma membrane to release the virion outside the cell. The fusogenic activity of viral glycoproteins may be needed for the initially enveloped particles to fuse with the outer nuclear membrane to be released into the cytoplasm.

Other than the fusogenic activity of CMV gB, the protein also initiates multiple signaling events, even in the absence of other virion components. CMV attachment and entry induce activation of innate immune responses, including the interferon response and inflammatory cytokine induction. gB has been demonstrated to bind Toll-like receptor 2, likely leading to the induction of inflammatory cytokines.

In some embodiments, a provided pharmaceutical composition (e.g., immunogenic composition, e.g., CMV vaccine) comprises or delivers an antigen that is or comprises CMV gB protein. In some embodiments, a provided pharmaceutical composition (e.g., immunogenic composition, e.g., CMV vaccine) comprises or delivers an antigen that is or comprises one or more portions of the gB protein (UL55) as listed in Tables 8A, Table 8B, and/or Table 9A.

### PUL86

In some embodiments, a provided composition (e.g., a pharmaceutical composition, e.g., immunogenic composition, e.g., vaccine) comprises or delivers a CMV pUL86 protein, or fragment or epitope thereof. In some embodiments, a polyribonucleotide as described herein encodes a polypeptide, where the polypeptide comprises one or more antigens, and where the one or more antigens comprises a CMV pUL86 protein, or fragment or epitope thereof. The capsid of CMV is defined by an icosahedral structure of 12 pentons, 150 hexons, and 320 triplexes. There are at least five major protein components of the capsid. One of these components, pUL86, is the most abundant protein component of the capsid with 960 copies. pUL86 forms the pentons and hexons necessary to the icosahedral structure. pUL86 is a late stage gene that is highly conserved and shares similarities with major capsid proteins of other viruses in the human population like Ebstein-Barr virus, herpes simplex virus type 1, varicellazpster virus, and human herpes virus 6. Infectious CMV virions contain a high proportion of UL86 relative to other proteins and has been shown to be one the most recognized targets of CD4 T-cells, an observation important to potential vaccine strategies.

### UL48-49

In some embodiments, a provided composition (e.g., a pharmaceutical composition, e.g., immunogenic composition, e.g., vaccine) comprises or delivers a CMV UL48 protein, or fragment or epitope thereof. In some embodiments, a provided pharmaceutical composition (e.g., immunogenic composition, e.g., vaccine) comprises or delivers a CMV UL49 protein, or fragment or epitope thereof. In some embodiments, a polyribonucleotide as described herein encodes a polypeptide, where the polypeptide comprises one or more antigens, and where the one or more antigens comprises a CMV UL48 protein, or fragment or epitope thereof. In some embodiments, a polyribonucleotide as described herein encodes a polypeptide, where the polypeptide comprises one or more antigens, and where the one or more antigens comprises a CMV UL49 protein, or fragment or epitope thereof.

UL48-49 also known as the smallest capsid protein (SCP) decorate the hexons of the capsid and plays an essential role in infectious virion assembly. UL48 and UL49 are also late stage genes and serve as a vital linker of inner and outer tegument components with the capsid. These proteins have a dynamic role in the CMV life cycle. They are involved in the targeting of the capsid at the nuclear pore complex and also help initiate subsequent uncoating after attachment.

### UL80a

In some embodiments, a provided composition (e.g., a pharmaceutical composition, e.g., immunogenic composition, e.g., vaccine) comprises or delivers a CMV pUL80a protein, or fragment or epitope thereof. In some embodiments, a polyribonucleotide as described herein encodes a polypeptide, where the polypeptide comp80a protein, or fragment or epitope thereof. UL80a encodes a protease (pUL80a) and an assembly protein (pUL80.5) which are both assembly protein precursors and essential to the generation and maturation of the capsid subunit. Specifically, it is the C-terminus of this protein constitutes the assembly component of this domain while the N-terminus has proteolytic activity.

### UL80.5

In some embodiments, a provided composition (e.g., a pharmaceutical composition, e.g., immunogenic composition, e.g., vaccine) comprises or delivers a CMV pUL80.5 protein, or fragment or epitope thereof. In some embodiments, a polyribonucleotide as described herein encodes a polypeptide, where the polypeptide comprises one or more antigens, and where the one or more antigens comprises a CMV pUL80.5 protein, or fragment or epitope thereof.

UL80.5 encodes a scaffolding protein that directly interacts with the Major Capsid Protein pUL86, facilitating the nuclear translocation of pUL86. In addition to this interaction, pUL80.5 is able to self-interact and lead to the generation of multimers, with, in conjunction with pUL86, is thought to lead to the formation of intranuclear hexons and pentons. Although this protein plays an essential role in capsid biogenesis, it has never been discovered within a mature capsid of a virion. This may lead to the conclusion that these proteins cannot be productive therapeutic targets; however, their necessity in capsid formation may make them an important target when the latent stage of CMV infection is reactivating to its lytic stage, where scaffolding proteins will be expressed and play an active role in increasing viremia.

pUL104

In some embodiments, a provided composition (e.g., a pharmaceutical composition, e.g., immunogenic composition, e.g., vaccine) comprises or delivers a CMV pUL104 protein, or fragment or epitope thereof. In some embodiments, a polyribonucleotide as described herein encodes a polypeptide, where the polypeptide comprises one or more antigens, and where the one or more antigens comprises a CMV pUL104 protein, or fragment or epitope thereof. The CMV capsid consists of a portal that exists at one of the 12 vertices of the capsid and is important for virus replications in all herpesviruses. pUL104 is a CMV dodecameric portal protein with 12-fold symmetry. pUL104 interacts with pUL56 to facilitate the essential process of DNA insertion into the capsid.

CMV Terminase Proteins pUL56, pUL51, and pUL89

In some embodiments, a provided composition (e.g., a pharmaceutical composition, e.g., immunogenic composition, e.g., vaccine) comprises or delivers a CMV pUL56 protein, or fragment or epitope thereof. In some embodiments, a provided pharmaceutical composition (e.g., immunogenic composition, e.g., vaccine) comprises or delivers a CMV pUL51 protein, or fragment or epitope thereof. In some embodiments, a provided pharmaceutical composition (e.g., immunogenic composition, e.g., vaccine) comprises or delivers a CMV pUL89 protein, or fragment or epitope thereof. In some embodiments, a polyribonucleotide as described herein encodes a polypeptide, where the polypeptide comprises one or more antigens, and where the one or more antigens comprises a CMV pUL56 protein, or fragment or epitope thereof. In some embodiments, a polyribonucleotide as described herein encodes a polypeptide, where the polypeptide comprises one or more antigens, and where the one or more antigens comprises a CMV pUL51 protein, or fragment or epitope thereof. In some embodiments, a polyribonucleotide as described herein encodes a polypeptide, where the polypeptide comprises one or more antigens, and where the one or more antigens comprises a CMV pUL89 protein, or fragment or epitope thereof.

In addition to proteins most outwardly facing in a mature virion, there are proteins which play a pivotal role in the assembly of the mature virion. Although these proteins may not have a role in immune activation by a mature virion, the essential role they play in viral assembly as well as their abundance, often as large complexes in different domains of an infected cell subject them to exposure to the robust network of cytosolic immune surveillance mechanisms universal among cell types and makes immune responses directed towards these proteins potentially efficacious at preventing viral assembly. This strategy could prove to be especially efficacious during the initial stages of latent stage reactivation into lytic stage infection, where an immune response primed to be activated at a time when viremia is low could halt the faithful activation of the lytic stage entirely. Proteins involved in viral DNA cleavage and packaging are a perfect example of these types of proteins. For example, HCMV terminase proteins form a hereto-oligomer complex of three core proteins, pUL56, pUL89, and pUL51, each with a different role in the essential process of DNA-packaging. pUL56 is arguable the most important constituent of the complex and is designated as the large terminase subunit of this complex. It is a 96 kDa, 850 amino acids protein with 12 conserved regions. pUL56 contains a nuclear localization signal that directs the terminase complex, after interaction with other terminase complex constituents, pUL89 and pUL51, to the nucleus of an infected host cell, allowing the complex to play its essential DNA packaging role. After the terminase complex enters the nucleus, pUL51 detects and directly interacts with the “pac” (cis-acting packaging signal) motifs of the viral genome. After this interaction and the creation of a hetero-oligomer terminase complex, the ATPase activity of pUL51 powers the translocation of a unit-length viral DNA into a procapsid. The Terminase small subunit of the terminase complex is pUL89 which is a 75 kDa protein containing a putative DNA binding domain with implicated nuclease activity. This is the only member of the terminase complex which has demonstrated nuclease activity in-vitro. In addition to this function, pUL89 has been shown to increase ATPase activity of pUL56 by 30%. The exact role pUL89 plays in the viral cycle has not been elucidated, but biologically speaking, nuclease activity of the terminal complex is required for the cleavage and release of viral DNA into the capsid, a process pUL89 may facilitate.

CI Complex Proteins eB(IL55)

In some embodiments, a provided composition (e.g., a pharmaceutical composition, e.g., immunogenic composition, e.g., vaccine) comprises or delivers a CMV CI complex protein, or fragment or epitope thereof. In some embodiments, a polyribonucleotide as described herein encodes a polypeptide, where the polypeptide comprises one or more antigens, and where the one or more antigens comprises a CI complex protein, or fragment or epitope thereof.

CMV has a highly complex viral envelope with a mosaic of covalently linked glycoproteins complexes required for CMV infectivity. There are four distinct types of covalently linked glycoprotein complexes required for CMV pathogenesis. One such complex known as complex I (CI) consists of glycoprotein B (gB), a major envelope glycoprotein known to be immunogenic and the target of neutralizing antibodies. GB is part of the major immediate early (MIE) genes which play an essential role in the reactivation of CMV from latency to acute phase of infection. Due to its variability across strains, gB is often used to establish CMV genotypes, with four gB variants described. Although not involved in the initial stages of attachment, gB is described to mediate the pH-dependent fusion process of the virion envelope with the cellular membrane. GB facilitates this fusion process through its interaction with THY-1, a host cargo protein involved in clatherin-independent endocytosis. Host surface pathogen recognition receptor (PRR) heterodimer TLR1/2 is known to detect triacyl lipopeptides and glycolipids and has been shown to directly bind to gB, initiating an immune response which leads to the stimulation of dendritic cells and the secretion of inflammatory cytokines that ultimately lead to the recruitment of NK cells. In addition to this surface PRR, cytosolic PRRs like AIM2 are capable of detecting the dsDNA of CMV and activate multiple immunological pathways including caspase activation, cell death via pyroptosis, Nf-kb activation and the production of pro-inflammatory cytokines.

### CII Complex Proteins Glycoprotein M (gM) and Glycoprotein N (N)

In some embodiments, a provided composition (e.g., a pharmaceutical composition, e.g., immunogenic composition, e.g., vaccine) comprises or delivers a CMV CII complex protein, or fragment or epitope thereof. In some embodiments, a polyribonucleotide as described herein encodes a polypeptide, where the polypeptide comprises one or more antigens, and where the one or more antigens comprises a CII complex protein, or fragment or epitope thereof.

CII complex proteins gM and gN are implicated in the initial stages of host cell interaction via their interaction with glycosaminoglycans like heparin sulfate proteoglycans on cell membranes. The importance of gM and gN in cell attachment has been shown through the observation that antibodies against them can neutralize and inhibit the progression of CMVs life cycle. Although gM and gN have been demonstrated to be viable therapeutic targets, they are lesser studied proteins of the viral envelope.

CIII Complex Proteins Glycoprotein H (gH), Glycoprotein L (gL), and Glycoprotein O (gO)

In some embodiments, a provided composition (e.g., a pharmaceutical composition, e.g., immunogenic composition, e.g., vaccine) comprises or delivers a CMV CIII complex protein, or fragment or epitope thereof. In some embodiments, a polyribonucleotide as described herein encodes a polypeptide, where the polypeptide comprises one or more antigens, and where the one or more antigens comprises a CIII complex protein, or fragment or epitope thereof.

The CIII complex consist consists of a covalently linked hetero-trimer comprised of several major immediate expression genes encoding the glycoproteins, gH, gL, and gO. This complex is important for CMV attachment and invasion via the activation of gB fusogenic activity by gH and gL in what has been shown to be an essential process for producing an infectious form of CMV. The precise role of gO is not clear, but it has been shown to be non-essential and likely functions as a co-receptor cooperating with the fusion-competent gH. In addition to their participation in CIII, gH and gL can also covalently interact with three smaller glycoproteins UL128, UL130, and UL131A to produce a heteropentamer. Similar to gB, variations in gH are also used to establish CMV genotypes with two known gH genotypes. Also similar to gB, gH can facilitate the viral fusion process through interaction with THY-1. This interaction helps define the tropism of CMV, as THY-1 is expressed in numerous cell types including fibroblast and epithelial cells.

In addition to these known glycoproteins, there are at least 20 putative non complexed enveloped proteins that have not been fully characterize but are potentially important for host cell interaction and infection.

### US28

In some embodiments, a provided composition (e.g., a pharmaceutical composition, e.g., immunogenic composition, e.g., vaccine) comprises or delivers a CMV US28 protein, or fragment or epitope thereof. In some embodiments, a polyribonucleotide as described herein encodes a polypeptide, where the polypeptide comprises one or more antigens, and where the one or more antigens comprises a CMV US28 protein, or fragment or epitope thereof.

An approach at detecting viral associated proteins and antigens may not exclusively rely on proteins specifically localized to the viral envelope, but also viral proteins which localize to the plasma membrane of infected host cells. This notion is especially significant in the case of latent stage CMV infection, when the virus exists in a largely dormant stage, with a significantly reduced expression profile. In this vein of reasoning, US28 represents an important protein as it is one of just a few viral proteins expressed during the latent stage of CMV infection, and experimentally proven to exist at the host cell surface. It encodes a cell surface G protein-coupled receptor that can bind and internalize several chemokines, allowing it to function as an immune signaling sink. Cytokine binding of US28 has only been demonstrated in the acute stage of infection; however, it is easy to appreciate the advantages modulating the immune landscape can have for latent stage persistence, as sequestration of chemokines can reduce local inflammation and immune cell targeting responses harmful to the virus. Evidence of host immune response modulation has been shown by US28's ability to down regulate MAP kinase and NF-kb activation. All of these immune related functions create a more permissive host environment for CMV to establish a latent infection. Furthermore, it has been shown that eliminating the immunosuppressive activities of US28 can lead CMV to reactivate to its lytic (acute) phase of infection, allowing pre-existing memory responses to recognize and respond to the once immunologically quiescent phase of infection, a fact of particular interest to therapeutic approaches effective only against actively replicated virus.

### UL11A

In some embodiments, a provided composition (e.g., a pharmaceutical composition, e.g., immunogenic composition, e.g., vaccine) comprises or delivers a CMV UL11A protein, or fragment or epitope thereof. In some embodiments, a polyribonucleotide as described herein encodes a polypeptide, where the polypeptide comprises one or more antigens, and where the one or more antigens comprises a CMV UL11A protein, or fragment or epitope thereof.

UL111A is another viral protein expressed during the latent stage of the virus and also shown to partially localize to the host cell surface. ULL11A encodes a viral IL-10 homologue which functions to downregulate MHC class II expression, inhibiting the ability of infected professional antigen presenting cells to present viral proteins in the context of MHC class II to CD4 T-cells. This function has significance in the production of durable memory response which necessitates CD4 activation and associated cytokine production. Furthermore, it can tilt the immunological axis to one of tolerance instead of activation, making persistence, and then eventual reactivation, more favorable for CMV. Furthermore, the preferential expression of UL111A, like US28, on the surface of chronically infected host cells can afford us with viable therapeutic targets to combat a stage of CV where viral proteins are scarce and thus hard to detect by immune surveillance and response mechanisms.

### Norovirus Protein Sequences

In some embodiments, a provided composition (e.g., a pharmaceutical composition, e.g., immunogenic composition, e.g., vaccine) comprises or delivers (e.g., causes expression of in a recipient organism, for example by administration of a nucleic acid construct, such as an RNA construct as described herein, that encodes it) an antigen that is or comprises one or more epitopes (e.g., one or more B-cell and/or one or more T-cell epitopes) of a norovirus protein. In some embodiments, a composition described herein induces a relevant immune response effective against norovirus (e.g., by targeting a norovirus protein).

In some embodiments, a provided composition (e.g., a pharmaceutical composition, e.g., immunogenic composition, e.g., vaccine) comprises or delivers an antigen that is or comprises a full-length norovirus protein. In some embodiments, a provided composition (e.g., a pharmaceutical composition, e.g., immunogenic composition, e.g., vaccine) comprises or delivers an antigen that is or comprises a portion of a norovirus protein that is less than a full-length norovirus protein. In some embodiments, a provided composition (e.g., a pharmaceutical composition, e.g., immunogenic composition, e.g., vaccine) comprises or delivers a chimeric polypeptide that is or comprises part or all of a norovirus protein and one or more heterologous polypeptide elements.

In some embodiments, an antigen that is included in and/or delivered by a provided pharmaceutical composition (e.g., immunogenic composition, e.g., norovirus vaccine) is or comprises one or more peptide fragments of a norovirus antigen; in some such embodiments, each of the one or more peptide fragments includes at least one epitope (e.g., one or more B cell epitopes and/or one or more T cell epitopes), for example as may be predicted, selected, assessed and/or characterized as described herein.

In some embodiments, an antigen that is included in and/or delivered by a provided pharmaceutical composition (e.g., immunogenic composition, e.g., norovirus vaccine) is or comprises a plurality of peptide fragments of one or more norovirus antigens. In some embodiments, a single polypeptide antigen may include a plurality of such fragments, e.g., presented as a string antigen as described herein.

In some embodiments, one or more norovirus epitopes (e.g., included in part or all of a norovirus protein) may be linked with one or more sequences with which it is linked in nature; in some such embodiments, such sequence(s) may be or comprise one or more heterologous elements (e.g., one or more elements, not naturally found in the relevant norovirus protein or not naturally found directly linked to the relevant norovirus epitope(s)). For example, in some embodiments, an antigen peptide provided and/or utilized in accordance with the present disclosure may include one or more linker elements and/or one or more membrane association elements and/or one or more secretion elements, etc. In some embodiments, an antigen peptide may comprise a plurality of norovirus protein fragments or epitopes separated from one another by linkers.

In some embodiments, a norovirus protein, or fragment or epitope thereof, utilized in an antigen as described herein may include one or more sequence alterations relative to a particular reference norovirus protein, or fragment or epitope thereof. For example, in some embodiments, a utilized antigen may include one or more sequence variations found in circulating strains or predicted to arise, e.g., in light of assessments of sequence conservation and/or evolution of norovirus proteins over time and/or across strains. Alternatively or additionally, in some embodiments, a utilized antigen may include one or more sequence variations selected, for example, to impact stability, folding, processing and/or display of the antigen or any epitope thereof.

In some embodiments, a norovirus protein, or fragment or epitope thereof, utilized in an antigen as described herein shows at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with a relevant corresponding reference (e.g., wild type) protein, fragment or epitope. In some embodiments, a norovirus protein, or fragment or epitope thereof, utilized in an antigen as described herein shows at least 80%, 81%, 82%, 83%, 84%, 85%, 86%, 87%, 88%, 89%, 90%, 91%, 92%, 93%, 94%, 95%, 96%, 97%, 98%, 99%, or 100% sequence homology (i.e., identity or conservative substitution as is understood in the art) amino acid sequence identity with a relevant corresponding reference (e.g., wild type) protein, fragment or epitope. Moreover, in some embodiments, a norovirus protein, or fragment or epitope thereof, utilized in an antigen as described herein shares conserved amino acid residues (e.g., at corresponding positions) with a relevant corresponding reference (e.g., wild type) protein, fragment or epitope. Those skilled in the art will appreciate that, in general, lower percent identity or homology may be tolerated for shorter peptides, as a single change will by definition have a larger impact on percent identity or homology when considered relative to a smaller number of residues. For example, those skilled in the art will appreciate that, for sequences longer than about 20 amino acids, percent identity or homology is typically greater than about 80%; for sequences longer than about 50 amino acids, percent identity or homology is typically greater than about 90%.

In some embodiments, assessments of degree of conservation may consider the physiochemical difference between two amino acids as described, for example, in WO2014/180569, which is incorporated herein by reference in its entirety. It is well known in molecular evolution that amino acids that interchange frequently are likely to have chemical and physical similarities whereas amino acids that interchange rarely are likely to have different physico-chemical properties. The likelihood for a given substitution to occur in nature compared with the likelihood for this substitution to occur by chance can measured by log-odds matrices. The patterns observed in log-odds matrices imposed by natural selection “reflect the similarity of the functions of the amino acid residues in their weak interactions with one another in the three dimensional conformation of proteins” (See Dayhoff et al. Atlas of protein sequence and structure 5:345, 1978, which is incorporated herein by reference in its entirety). In some embodiments, evolutionary based log-odds matrices, which may be referred to as “T scores” can be used to reflect extent to which a sequence variation might impact T cell recognition. Substitutions with positive T scores (i.e., log-odds) are likely to occur in nature, and hence correspond to two amino acids that have similar physico-chemical properties. Substitutions with positive T scores would have a lower likelihood of altering immunogenicity. Conversely, substitutions with negative T scores reflect substitutions that are unlikely to occur in nature and hence correspond to two amino acids that have significantly different physico-chemical properties. Such substitutions would have a greater chance of altering immunogenicity. In some embodiments, presence of negative T score substitutions within a sequence, even if it is otherwise highly conserved, may indicate that it would be relatively less useful in a vaccine antigen as described herein.

In some embodiments, a utilized antigen induces an immune response that targets a VP protein, such as a VP1 protein (e.g., an S domain and/or a P domain, such as a P2 domain, thereof). In some embodiments, a utilized antigen induces an immune response that targets a VP1 protein from any of genogroups and/or genotypes. In some embodiments, a utilized antigen induces an immune response that targets a VP1 protein from GI or GII. In some such embodiments, an immune response may be or comprise a T cell immune response.

In some embodiments, a utilized antigen is or comprises one or more norovirus protein sequences (e.g., conserved sequences and/or sequences that are or comprise one or more B cell epitopes and/or one or more CD4 epitopes and/or one or more CD8 epitopes) of an antigen expressed Certain B cell and T cell epitopes have been described for noroviruses of various genogroups (see, for example, van Loben Sels & Green, Viruses 11:432, 2019, doi:10.3390/v11050432, which is incorporated herein by reference in its entirety).

In some embodiments, a utilized antigen is or comprises one or more norovirus protein sequences found in a strain that is circulating or has circulated in a relevant region (e.g., where subjects to be vaccinated are or will be present). It is noted, for example that GII.4 viruses have caused the majority of norovirus outbreaks worldwide, although in recent years, non-GII.4 viruses, such as GI.17 and GII.2, have temporarily replaced GII.4 viruses in several Asian countries. Between 2002 and 2012, new GII.4 viruses emerged about every 2 to 4 years, but since 2012, the same virus (GII.4 Sydney) has been the dominant strain worldwide.

In some embodiments, an antigen utilized in accordance with the present disclosure includes norovirus protein sequences identified and/or characterized by one or more of:
HLA-I or HLA-II binding (e.g., to HLA allele(s) present in a relevant population)
HLA ligandomics data, optionally confirmed by mass spectrometry
Relatively high expression
Sequence conservation
Surface exposure
Serum reactivity
Immunogenicity (e.g., presence of one or more B-cell and/or T-cell epitopes; evidence of ability to induce sterile protection in model systems including, e.g., humans, non-human primates, mice, zebrafish larvae and/or cell lines; certain models are described, for example, in Makimaa et al., Viruses 12:904, 2020, doi:10.3390/v12080904, which is incorporated herein by reference in its entirety).
Absence of sequences that overlap with human proteome

In some embodiments, such characteristics are experimentally or computationally assessed. In some embodiments, such characteristics are assessed by consultation with published reports.

For example, in some embodiments, HLA-I and/or HLA-II binding is experimentally assessed; in some embodiments it is predicted.

In some embodiments, predicted HLA-I or HLA-II binding is assessed using an algorithm such as neonmhc 1 and/or neonmhc2, which predict and/or characterize likelihood of MHC class I and MHC class II binding, respectively. Alternatively or additionally, in some embodiments, an MHC-peptide presentation prediction algorithm or MHC-peptide presentation predictor is or comprises NetMHCpan or NetMHCIIpan. In some embodiments, a hidden markov model approach may be utilized for MHC-peptide presentation prediction and/or characterization. In some embodiments, the peptide prediction model MARIA may be utilized. In some embodiments, NetMHCpan is not utilized to predict or characterize likelihood of MHC binding for peptides as described herein. In some embodiments, the peptide prediction model MARIA may be utilized. In some embodiments, NetMHCIIpan is not utilized to predict or characterize likelihood of MHC binding for peptides as described herein. In some embodiments, neither NetMHCpan nor NetMICIIpan is utilized to predict or characterize likelihood of MHC binding for peptides as described herein. In some embodiments, an MHC-peptide presentation prediction algorithm or MHC-peptide presentation predictor is or comprises RECON® (Real-time Epitope Computation for ONcology), which offers high quality MHC-peptide presentation prediction based on expression, processing and binding capabilities. See, for example, Abelin et al., Immunity 21:315, 2017; Abelin et al., Immunity 15:766, 2019, each of which is incorporated herein by reference in its entirety.

In some embodiments, HLA binding and/or ligandomics assessments will consider the geographic region of subjects to be immunized. For example, in some embodiments, HLA allelic diversity will be considered. In some embodiments, antigen(s) included in a provided pharmaceutical composition (e.g., immunogenic composition, e.g., vaccine) will be or comprise peptides (e.g., epitopes) expected or determined, when considered together, to bind to a significant percentage (e.g., at least 60%, 65%, 70%, 75%, 80%, 85%, 90%, 95%, or more) of HLA alleles expected or known to be present in a relevant region or population. In some embodiments, antigen(s) included in a provided pharmaceutical composition (e.g., immunogenic composition, e.g., vaccine) will be or comprise peptides expected or determined, when considered together, to bind to the most prevalent (e.g., the 1, 2, 3, 4, 5, 6, 7, 8, 9, or 10 most prevalent, or at least 1, 2, 3, 4, or 5 of the 10 most prevalent, etc.) HLA alleles expected or known to be present in a relevant region or population).

In some embodiments, expression level is experimentally determined (e.g., in a model system or in infected humans). In some embodiments, expression level is a reported level (e.g., in a published or presented report). In some embodiments, expression level is assessed as RNA (e.g., via RNASeq). In some embodiments (and typically preferably), expression levels is assessed as protein.

In some embodiments, sequence conservation is assessed, for example, using publicly available sequence evaluation software (such as, for example, multiple sequence alignment programs MAFFT, Clustal Omega, etc.). In some embodiments, sequence conservation is determined by consultation with published resources (e.g., sequences). In some embodiments, sequence conservation includes consideration of currently or recently detected strains (e.g., in an active outbreak).

In some embodiments, surface exposure is assessed by reference to publicly available database and/or software. In some embodiments, surface exposure is assessed by reference to publicly available data, e.g., as described in Allen et al. PLoS ONE 3(1): e1485 (2008); Zhang et al., Archives of Virology 164:1629 (2019); Allen et al., Virol. J 6:150 (2009), each of which is incorporated herein by reference in its entirety for purposes described herein.

In some embodiments, serum reactivity is assessed by contacting serum samples from infected individuals with polypeptides including sequences of interest (e.g., as may be displayed via, for example, phage display or peptide array, etc; see, for example, Whittemore et al “A General Method to Discover Epitopes from Sera” PlosOne, 2016; https://doi.org/10.1371/journal.pone.0157462, which is incorporated herein by reference in its entirety). In some embodiments, serum reactivity is assessed by consultation with literature reports and or database data indicating serum-recognized sequences.

In some embodiments, assessment of immunoreactivity and/or of presence of an epitope may be or comprise consultation with the Immune Epitope Database (IEDB) which those skilled in the art will be aware is a freely available resource funded by NIAID that catalogs experimental data on antibody and T cell epitopes (see iedb.org).

In some embodiments, ability to induce sterile protection is assessed, for example, as described in one or more of Pattekar et al. Cell Mol Gastroenterol Hepatol 11:1267 (2021); Malm et al., Scientific Reports, 9:3199 (2019); and Esposito et al., Front. Immunol. 11:1383 (2020), each of which is incorporated herein by reference in its entirety for purposes described herein.

In some embodiments, antigen(s) utilized in accordance with the present disclosure are characterized by dendritic cell presentation which, in turn may be indicative of HLA binding and/or of immunogenicity. In some embodiments, antigen(s) utilized in accordance with the present disclosure are or comprises sequences (e.g., epitopes, fragments, complete proteins) of norovirus proteins that include blockade epitopes and/or T cell stimulatory epitopes e.g., as described herein (see also van Loben Sels & Green, Viruses 11:432, 2019, doi: 10.3390/v11050432, which is incorporated herein by reference in its entirety) demonstrated.

In some embodiments, an antigen utilized in accordance with the present disclosure an antigen is or comprises a norovirus VP protein selected from the group consisting of VP1 and VP2, and variants thereof and/or fragments or epitopes of any of the foregoing, and combinations of any of the foregoing.

In some embodiments, an antigen utilized in accordance with the present disclosure is or comprises a norovirus protein selected from the group consisting of a NoV VP1, a NoV VP2, a NoV N-terminal protein (NS1 and/or NS2), a NoV NTPase (NS3), a NoV P22 (NS4), a NoV VPg (NS5), a NoV Protease (NS6), a NoV Polymerase (NS7), and variants thereof and/or fragments or epitopes of any of the foregoing, and combinations of any of the foregoing. In some embodiments, an antigen utilized in accordance with the present disclosure is or comprises a norovirus VP1 protein or variant thereof or one or more fragments or epitopes of such VP1 protein or variant thereof(e.g., used individually or in combination (e.g., as part of a multiepitope construct, such as a string construct, as described herein) with one another and/or with one or more other norovirus proteins or fragments or epitopes thereof). In some embodiments, an antigen utilized in accordance with the present disclosure is or comprises a norovirus VP1 protein of norovirus genogroup GI or variant thereof or one or more fragments or epitopes of such VP1 protein or variant thereof (e.g., used individually or in combination (e.g., as part of a multiepitope construct, such as a string construct, as described herein) with one another and/or with one or more other norovirus proteins or fragments or epitopes thereof, for example from the same or different genogroups and/or genotypes). In some embodiments, an antigen utilized in accordance with the present disclosure is or comprises a norovirus VP1 protein of norovirus genogroup GII or variant thereof or one or more fragments or epitopes of such VP1 protein or variant thereof (e.g., used individually or in combination (e.g., as part of a multiepitope construct, such as a string construct, as described herein) with one another and/or with one or more other norovirus proteins or fragments or epitopes thereof, for example from the same or different genogroups and/or genotypes).

Literature includes reports of certain T cell epitopes within VP1 (see van Loben Sels & Green, Viruses 11:432, 2019, doi:10.3390/v11050432, which is incorporated herein by reference in its entirety).

Among other things, the present disclosure provides an insight that, in some embodiments, it may be desirable to include two or more different epitopes, optionally from two or more different norovirus proteins, in pharmaceutical composition (e.g., immunogenic composition, e.g., vaccine) compositions, which can be useful in the treatment of norovirus. The present disclosure identifies the source of a problem with various strategies for norovirus vaccination, including that robust protection has not been achieved, particularly across variants, strains, clades, and/or genogroups. Without wishing to be bound by any particular theory, proposes that inclusion of a plurality of epitopes, for example in a non-natural format (e.g., a string format as described herein) and/or that are from different norovirus protein, may achieve more potent protection than that currently observed, for example, with other vaccine formats.

### Viral Protein 1 (VP1)

In some embodiments, a provided composition (e.g., a pharmaceutical composition, e.g., immunogenic composition, e.g., vaccine) comprises or delivers a norovirus VP1 protein, or fragment or epitope thereof. In some embodiments, a polyribonucleotide as described herein encodes a polypeptide, where the polypeptide comprises one or more antigens, and where the one or more antigens comprises a norovirus VP1 protein, or fragment or epitope thereof.

The term “VP1 antigen” may be used herein to refer to an antigen that includes at least one VP1 fragment (e.g., an S domain fragment or P domain fragment) or epitope (e.g., B cell or T cell epitope, e.g., an S domain or P domain B cell or T cell epitope).

In some embodiments, a provided pharmaceutical composition (e.g., immunogenic composition, e.g., vaccine) comprises or delivers a full-length VP1 protein or variant thereof. In some embodiments, a provided pharmaceutical composition (e.g., immunogenic composition, e.g., vaccine) comprises or delivers a fragment (e.g., a fragment that is or comprises an S domain or a P domain, or a fragment or epitope of either of the foregoing, such as a P1 or P2 subdomain or fragment or epitope thereof) of a VP1 protein or variant thereof. In some embodiments, a provided pharmaceutical composition (e.g., immunogenic composition, e.g., vaccine) comprises or delivers a VP1 antigen (e.g., a full length or fragment VP1, or a variant thereof) separately (e.g., from a separate RNA and/or a separate LNP) from at least one other antigen (e.g., a multi-epitope antigen) as described herein.

In some embodiments, a provided pharmaceutical composition (e.g., immunogenic composition, e.g., vaccine) comprises or delivers a polypeptide that is or comprises a P domain such as a P2 domain. In some embodiments, P domain sequences (e.g., P2 domain sequences) are selected that are expected or known to elicit antibodies, e.g., antibodies that interfere with HBGAs interaction.

In some embodiments, a provided pharmaceutical composition (e.g., immunogenic composition, e.g., vaccine) comprises or delivers antigen(s) that is/are or comprise a plurality of P2 domains of different sequences (e.g., in some embodiments representing different viral variants that, for example, may have been detected or expected in a particular region or population and/or according to observed or expected mutation trends, and/or that may have been expected or predicted, together, to induce or support an immune response that includes antibodies and/or T cells that bind to and/or otherwise are effective against (e.g., that block capsid formation and/or viral entry, and/or that target virus-infected cells) a plurality of viral strains or variants.

In some embodiments, a provided pharmaceutical composition (e.g., immunogenic composition, e.g., vaccine) comprises or delivers a polypeptide including a VP1 epitope that is bound by monoclonal antibody NV8812 (see White et al. J Virol. 70:6589-97. doi: 10.1128/JVI.70.10.6589, 1996, which is incorporated herein by reference in its entirety).

In some embodiments, a provided pharmaceutical composition (e.g., immunogenic composition, e.g., vaccine) comprises or delivers a polypeptide a polypeptide including a VP1 epitope from any genogroup and/or genotype of norovirus. In some such embodiments, a VP1 epitope may be from GI genogroup of norovirus. In some embodiments, a VP1 epitope may be from GII genogroup of norovirus.

Norovirus belongs to the Caliciviridae family of viruses which are non-enveloped, single stranded RNA viruses. Due to its non-enveloped structure, the capsid is the most immune facing component of the virus and as such, is the first to interact with humoral and cellular immune surveillance and response mechanisms. This fact makes the capsid an important target of vaccination strategies. The norovirus capsid is composed of 90 capsid protein monomers that form a T=3 icosahedral capsid. The 58 kDa monomer which comprises the capsid, named VP1, is encoded by the 2^{nd} of 3 total open-reading frames (ORF) that the norovirus genome contains, and is divided into 4 domains, the N-terminus (N), the shell (S) and C-terminal protruding (P) domains, each with varying structural, functional, and immunological significance. The monomeric construction of norovirus capsids may seem to present a predictable target for an immune response across all norovirus strains; however, the domain which interacts with host receptors and are considered of greatest physiological importance, the P domain, are highly polymorphic across norovirus strains. This variability is likely due to the high selective pressure imposed by immune responses which can disrupt receptor-ligand interactions essential for norovirus's life cycle and pathogenesis. Therefore, an analysis of the VP1's significance cannot be done without considering the sum of its parts.

### Viral Protein 2 (VP2)

In some embodiments, a provided composition (e.g., a pharmaceutical composition, e.g., immunogenic composition, e.g., vaccine) comprises or delivers a norovirus VP2 protein, or fragment or epitope thereof. In some embodiments, a polyribonucleotide as described herein encodes a polypeptide, where the polypeptide comprises one or more antigens, and where the one or more antigens comprises a norovirus VP2 protein, or fragment or epitope thereof.

VP2 is the only other structural protein besides VP1 that norovirus expresses. It is encoded by ORF3, has a molecular weight of 29 kDa, and is located inside of the viral capsid. VP2 interacts with VP1 via a highly conserved isoleucine residue (S domain residue 52, according to canonical numbering systems) in its IDPWI motif (see, Vongpunsawad et al. J Virol. 87:4818, (2013), doi: 10.1128/JVI.03508-12) an interaction with contributes to overall capsid stability. VP2 is also reported to interact with host restriction factors (see Cotten et al. J Virol. 88:11056, 2014, doi: 10.1128/JVI.01333-14, which is incorporated herein by reference in its entirety). It has been reported that absence of VP2 decreases stability and homogeneity of norovirus capsids or virus-like particles, and furthermore, that co-expression of VP1 and VP2 increases their expression relative to when they are separately expressed (see, for example, Vongpunsawad et al. J Virol. 87:4818, (2013), doi: 10.1128/JVI.03508-12; Liu et al. Arch Virol. 164:1173, (2019) doi: 10.1007/s00705-019-04192-2, each of which is incorporated herein by reference in its entirety). In addition to this more clearly defined role, VP2 is believed to play a role in RNA binding and genome packaging in nascent virions. Although VP2 typically remains less accessible to host immune surveillance mechanisms, it interestingly has been shown to have a greater mutations rate relative to VP1 as a whole, which implies an exposure to selective pressure through interaction with unknown host factors. A recently growing area of inquiry and one of great immunological significance is VP2's capacity to disrupt antigen presentation in the cells it infects. This capacity is of even greater significance when considering the ability of norovirus to infect professional antigen presenting cells such as macrophages. The mechanism which modulates this pathway is not entirely clear, but its significance to therapeutic strategies which utilize the antigen presenting capacity of cells may be important.

### S Domain

In some embodiments, a provided composition (e.g., a pharmaceutical composition, e.g., immunogenic composition, e.g., vaccine) comprises or delivers a norovirus S domain, or fragment or epitope thereof. In some embodiments, a polyribonucleotide as described herein encodes a polypeptide, where the polypeptide comprises one or more antigens, and where the one or more antigens comprises a norovirus S domain, or fragment or epitope thereof.

The S domain spans the most N-terminal region of the ORF that encodes VP1 and extends to residue number 255. Its name comes from the fact that it constitutes the interior shell of the capsid, which is predominantly occluded, and thus more immunologically inaccessible, by the protrusions which define the P domain. Nonetheless, this domain is essential for the assembly and stability of the icosahedral geometry of the capsid, and likely due to this, is the most conserved domain of VP1 among different norovirus strains. Icosahedral contacts between Vp1 dimers are modulated by the S domain, an interaction that has been shown to be necessary and sufficient to produce viral like particles (VLPs) which are a highly immunogenic structure morphologically and antigenically resembling whole virus devoid of genetic material, a potentially useful structure as an immunization platform.

### P Domain

In some embodiments, a provided composition (e.g., a pharmaceutical composition, e.g., immunogenic composition, e.g., vaccine) comprises or delivers a norovirus P domain, or fragment or epitope thereof. In some embodiments, a polyribonucleotide as described herein encodes a polypeptide, where the polypeptide comprises one or more antigens, and where the one or more antigens comprises a norovirus P domain, or fragment or epitope thereof.

The P domain starts at residue 223 and extends to the c-terminal end of the ORF encoding VP1. It is the domain that protrudes from the capsid and is of greatest importance for cell attachment and antigenicity. Structurally, it contributes to the control of the size and the stability of the norovirus capsid by comprising the intermolecular contacts between dimeric VP1 subunits. It is the P domain which binds host histo-blood group antigens (HBGAs) and defines the cellular tropism of norovirus. Due to its importance in receptor interaction and its protrusion from the capsid, the P-domain is the most immunologically exposed domain under the greatest selective pressure relative to the other domains of VP1, as an immune response directed to this domain can be highly efficacious in disrupting the life cycle of norovirus. This selective pressure has resulted in the greatest antigenic variation of any domain of VP1 and has defined one of the greatest challenges to norovirus vaccination, as the antigenicity of the P region focuses host memory immune response towards it, which may result in neutralization of one norovirus strain, but a lack of efficacy in another.

### P1

In some embodiments, a provided composition (e.g., a pharmaceutical composition, e.g., immunogenic composition, e.g., vaccine) comprises or delivers a norovirus P1 subdomain, or fragment or epitope thereof. In some embodiments, a polyribonucleotide as described herein encodes a polypeptide, where the polypeptide comprises one or more antigens, and where the one or more antigens comprises a norovirus P1 subdomain, or fragment or epitope thereof.

The p domain is comprised of two subdomains, named P1 and P2. The P1 subdomain consists of residues 226-278 and 406-520 of the P domain. The P1 domain, relative to P2, is the more conserved domain. The reason for the relative high conservation of the P1 domain is unclear; however, it is thought to be due to the essential role residues of this domain play in mediating interactions between individual capsid molecules, imposing a structural limit to the variation that can occur. CD8 T cell response has been shown to be specific to this domain in murine models, a promising finding considering the lack of variation in this region.

### P2

In some embodiments, a provided composition (e.g., a pharmaceutical composition, e.g., immunogenic composition, e.g., vaccine) comprises or delivers a norovirus P2 subdomain, or fragment or epitope thereof. In some embodiments, a polyribonucleotide as described herein encodes a polypeptide, where the polypeptide comprises one or more antigens, and where the one or more antigens comprises a norovirus P2 subdomain, or fragment or epitope thereof.

The P2 domain consists of residues 278-406 of the P domain and is inserted into the P1 domain. The P2 domain protrudes the most from the capsid, interacts with host receptors, and contains the greatest diversity across strains. Two sites within the P2 region susceptible to amino acid substitutions appear to be involved in the rise of variants associated with epidemics. Because of this high level of variation, immunization strategies most likely to overcome norovirus's ability to evade host immune responses will foreseeably involve a combinatorial approach targeting antigens most topographically exposed and important in host interactions, albeit at the expense of higher variability, with antigens that are less prone to antigenic variability but with the cost of lesser topographical exposure, like the P2 and S domain.

### Non-Structural Proteins

In some embodiments, a provided composition (e.g., a pharmaceutical composition, e.g., immunogenic composition, e.g., vaccine) comprises or delivers a norovirus non-structural protein, or fragment or epitope thereof. In some embodiments, a polyribonucleotide as described herein encodes a polypeptide, where the polypeptide comprises one or more antigens, and where the one or more antigens comprises a norovirus non-structural protein, or fragment or epitope thereof.

Non-structural proteins (NPS) are concealed by the capsid and thus have less immunological exposure relative to capsid proteins, but are none-the-less immunologically important. NPSs are expressed and assembled in an intracellular environment rich with pathogen recognition receptors and an immunoproteasome capable of loading peptides onto MHC class 1 for CD8 T-cell presentation, giving them transient exposure to host surveillance mechanisms before intact norovirus are formed. This intracellular exposure can be especially important in one of the proposed mechanism of norovirus persistence, whereby a greater proportion of norovirus remain within intact cells, giving greater opportunity for the robust intracellular network of immune surveillance mechanisms to detect the presence of NPSs expressed during assembly. The following is a summary of NSPs and the significance of each.

N-Terminal Protein (NS1-2; p48)

In some embodiments, a provided composition (e.g., a pharmaceutical composition, e.g., immunogenic composition, e.g., vaccine) comprises or delivers a norovirus P48 protein, or fragment or epitope thereof. In some embodiments, a polyribonucleotide as described herein encodes a polypeptide, where the polypeptide comprises one or more antigens, and where the one or more antigens comprises a norovirus P48 protein, or fragment or epitope thereof.

P48 is a non-structural protein with a molecular weight that ranges from 37-48 kDA depending on the strain that is expressing this protein. Furthermore, in a mouse model, vaccination of mice using this protein has been shown to protect mice norovirus infection. Interestingly, this protein has been shown to localize to and can induce the disassembly of the Golgi, a process which is believe to disrupt protein secretion by the host. To add evidence to the secretory pathway disruption P48 can induce, P48 has been shown to bind vesicle-associated-membrane protein-associated protein-A (VAP-A), an important factor in SNARE mediated vesicular transport. This disruption may potentially prevent viral derived glycoproteins from being transported to the cell surface, as has been shown in other viruses expressing homologues to P48. In consideration of the important host manipulating activities P48 can participate in, it is not surprising to learn that its importance in persistence has been demonstrated in murine models and pinpointed to a specific domain within P48. It is possible the modulation of immune response, couples with the inhibition of the secretory pathway, may promote a more immunologically tolerable form of infection that paves way to persistence.

The noroviral p48 protein, which is located at the N-terminus of the viral polyprotein, is characteristic of its genus; sequence comparisons across genogroups have revealed that the HuNoV Sydney p48 shares 42% identity with NV p48 (GI), 36% with Jena p48 (GIII), and 37% with MNV (GV) (Lateef et al., BMC Genomics. 18:39, 2017, doi: 10.1186/s12864-016-3417-4, which is incorporated herein by reference in its entirety). This low sequence similarity would seem to make p48 a poor therapeutic target universal among all strains; however, in-situ analysis has shown this protein to share core features across all strains including
(1) a disordered region of proline-rich N-terminal, containing the alleged immunogenic regions for MHC-I binding,
(2) a transmembrane hydrophobic domain at the C-terminal end,
(3) H-box and NC sequence motifs of the permutated NIpC/P60 family of circular peptidases that adapt different enzyme capacities within the same structure, improving the stability and the reducing degradation caused by proteases, and
(4) caspase cleavage and phosphorylation sites, which in eukaryotic cells are involved in the regulation of the cell cycle, apoptosis, and activation of the immune system:

Functionally, the p48 protein has been reported, when expressed in mammalian cells, to interfere in many immune signaling and activation pathways, such as those involving the Jak-STAT, MAPK, p53, and PI3K-Akt signaling pathways, and also to interfere with apoptosis, Toll-like receptors (TLR) signaling pathways, and the production of chemokines and cytokines (Lateef et al., BMC Genomics. 18:39, 2017, doi: 10.1186/s12864-016-3417-4, which is incorporated herein by reference in its entirety). These features are believed to afford norovirus with host manipulating characteristics including the manipulation of cell cycle, apoptosis, and activation of the immune system. Interestingly, this protein has been shown to localize to and can induce the disassembly of the Golgi, a process which is believe to disrupt protein secretion by the host. To add evidence to the secretory pathway disruption P48 can induce, P48 has been shown to bind vesicle-associated-membrane protein-associated protein-A (VAP-A), an important factor in SNARE mediated vesicular transport. This disruption may potentially prevent viral derived glycoproteins from being transported to the cell surface, as has been shown in other viruses expressing homologues to P48 The universality of the structural features which lead to these broadly important characteristics make P48 a promising target for therapeutic intervention.

The p48 protein thus (i) assists assembly of the replication complex; (ii) hampers certain cellular signaling pathways, (iii) inhibits activation of the immune response induced by viral infection, and can disrupt the host secretory pathway. Furthermore, in a mouse model, vaccination of mice using this protein has been shown to protect mice from norovirus infection. In consideration of the important host manipulating activities P48 can participate in, it is not surprising to learn that its importance in persistence has been demonstrated in murine models and pinpointed to a specific domain within P48. It is possible the modulation of immune response, coupled with the inhibition of the secretory pathway, may promote a more immunologically tolerable form of infection that paves way to persistence.

### NTPase (NS3)

In some embodiments, a provided composition (e.g., a pharmaceutical composition, e.g., immunogenic composition, e.g., vaccine) comprises or delivers a norovirus NS3 protein, or fragment or epitope thereof. In some embodiments, a polyribonucleotide as described herein encodes a polypeptide, where the polypeptide comprises one or more antigens, and where the one or more antigens comprises a norovirus NS3 protein, or fragment or epitope thereof.

The NTPase protein, also known as NS3, is a 40 kDa protein generated by cleavage of the polyprotein, in which it is located between residues 331 and 696, according to canonical numbering systems.

NS3 shows significant homology to the Enterpvirus 2C protein (see, Pfister et al. J Virol. 75:1611, 2001, doi: 10.1128/JVI.75.4.1611-1619.2001, which is incorporated herein by reference in its entirety). NS3 has a dynamic localization likely attributed to its dynamic role, localizing to vesicular and non-vesicular structures within the cytoplasm, the cellular ER membrane, and in some genotypes, the membrane of the mitochondria. Furthermore, this protein has been associated with the host secretory pathway and with lipid storage. Its dynamic localization is only exceeded by its diverse function, whereby it has been shown to bind and hydrolyze nucleoside triphosphates (NTP). NS3 has been reported to have enzymatic activity including (a) NTP-dependent helicase activity for unrolling RNA helices; (b) NTP-independent chaperone activity for remodeling of RNA structure and facilitating annealing of RNA chains, and (c) support of RNA synthesis by NS7. Co-expression of p48 and/or p22 has been reported to enhance NS3 activity, including specifically apoptotic activity (see, Yen et al. J Virol. 92:17, 2018, doi: 10.1128/JVI.01824-17, which is incorporated herein by reference in its entirety). The myriad of functions and sub-cellular localizations make this an important protein in the life-cycle of norovirus

### P22 (NS4)

In some embodiments, a provided composition (e.g., a pharmaceutical composition, e.g., immunogenic composition, e.g., vaccine) comprises or delivers a norovirus P22 protein, or fragment or epitope thereof. In some embodiments, a polyribonucleotide as described herein encodes a polypeptide, where the polypeptide comprises one or more antigens, and where the one or more antigens comprises a norovirus P22 protein, or fragment or epitope thereof.

The norovirus p22 protein, also known as P20 or NS4, is a 20-22 kDa protein that is a produced by cleavage of the ORF1 encoded preprotein. The mutation rate of this protein exceeds that of the complete genome and qualifies it as one of the most variable genomic regions of this strain. P22 includes a motif (YXϕESDG motif (36), which has been reported to mimic an export signal and can prevent COPII coated vesicles from successfully fusing with the trans-Golgi network, disrupting protein secretion and post-translational modification pathways vital to host cellular function. This endocytic pathway disrupting motif is highly conserved relative to the high variability of the P22 domain as a whole, emphasizing the physiological importance of this motif. Experiments have shown that P22 contributes to membrane alterations which, in concert with other non-structural proteins, enable the formation of the viral replication complex. (Sharp et al. PLoS ONE, 5:e13130, 2010, doi: 10.1371/journal.pone.0013130, which is incorporated herein by reference in its entirety). Immunologically, P22 can act as antagonist to immune responses by disrupting interferon and cytokine mediated signaling pathways typically activated in response to norovirus infection. Its involvement in creating the intracellular niche that is the replication complex makes it an appealing target as its disruption could unlock a treasure trove of antigenic targets typically in refuge within this replicative niche.

It has been reported that P22:
(1) acts as an antagonist in Golgi-dependent protein secretion (36); and
(2) acts as an antagonist of the immune response by altering the interferon and other cytokine signaling pathways after viral infection; and
(3) it promotes assembly of the viral replication complex.

### VPg (NS5)

In some embodiments, a provided composition (e.g., a pharmaceutical composition, e.g., immunogenic composition, e.g., vaccine) comprises or delivers a norovirus VPg protein, or fragment or epitope thereof. In some embodiments, a polyribonucleotide as described herein encodes a polypeptide, where the polypeptide comprises one or more antigens, and where the one or more antigens comprises a norovirus VPg protein, or fragment or epitope thereof.

The norovirus 15.5 kDa VPg protein is also generated by cleavage of the initial polyprotein (where it is found between residues 876 and 1008 according to the canonical numbering system). Norovirus VPg's most famous role is its covalent interaction with the 5′ end of the viral RNA genome, serving as a genome-linked protein. Importantly, VPg can participate in the initiation of viral RNA translation via the binding to cell translation interaction factor eIF3 and interaction with the cap-binding complex eIF4F, an essential helper function as norovirus mRNA lacks a coding region for a cap structure and internal ribosome binding site (Belliot et al. Virology 374:33, 2008, doi: 10.1016/j.virol.2007.12.028, which is incorporated herein by reference in its entirety) and/or by recruiting host elongation factor(s) (Daughenbaugh et al. EMBO J. 22:2852, 2003, doi: 10. 1093/emboj/cdg251, which is incorporated herein by reference in its entirety) (Hosmillo et al. Elife. 8:e46681, 2019, doi: 10.7554/eLife.46681, which is incorporated herein by reference in its entirety).

Also, a conserved conserved KGKxKxGRG motif found in the N-terminal region of the VPg proteins of all norovirus genogroups, except for GIII has been reported to be involved in inducing cell cycle arrest (in G1/G0) in infected cells (McSweeney et al. Viruses. 11:217, 2019, doi: 10.3390/v11030217, which is incorporated herein by reference in its entirety).

### Protease (NS6)

In some embodiments, a provided composition (e.g., a pharmaceutical composition, e.g., immunogenic composition, e.g., vaccine) comprises or delivers a norovirus NS6 protein, or fragment or epitope thereof. In some embodiments, a polyribonucleotide as described herein encodes a polypeptide, where the polypeptide comprises one or more antigens, and where the one or more antigens comprises a norovirus NS6 protein, or fragment or epitope thereof.

NS6, also known as 3CL Pro, is a 19.4 kDa protein involved in the cleavage process of the poly protein derived from the norovirus genome. NS6 has been characterized to have chymotrypsin like activity with an optimum function at a pH of 8.6. This protease is essential for the maturation of viral proteins like NS7 and P22. The norovirus protease protein (NS6) cleaves the polyprotein encoded by ORF1 via a two-stage process in which “early” sites (p48/NTPase and NTPase/p22) are cleaved first, followed by “late” sites (p22/VPg, Vpg/Pro, and Pro/Pol); it is worth noting that the ProPol precursor protein itself also shows cleavage ability, which has been reported to be comparable to that of the Pro protein alone (May et al. Virology 444:218, 2013, doi: 10.1016/j.virol.2013.06.013, which is incorporated herein by reference in its entirety). Some differences have been reported between Pro proteins of different genotypes. Specifically, the GII.4 protease crystal structure reveals differences in the substrate binding pocket and catalytic triad residues relative to that of the GI protein. The GII.4 protease active site also includes a conserved arginine residue that interacts with the catalytic histidine (Viskovska et al. J Virol. 93:e01479, 2019, doi: 10.1128/JVI.01479-18, which is incorporated herein by reference in its entirety). Interestingly and of pathological significance, this interaction can prevent access of the active site from protease inhibitors or substrates that may inactivate its activity. This molecular configuration potentially protects NS6's protease activity from protease inhibitors naturally produced in the location where norovirus infection is most commonly established, the GI tract.

### Polymerase (NS7)

In some embodiments, a provided composition (e.g., a pharmaceutical composition, e.g., immunogenic composition, e.g., vaccine) comprises or delivers a norovirus NS7 protein, or fragment or epitope thereof. In some embodiments, a polyribonucleotide as described herein encodes a polypeptide, where the polypeptide comprises one or more antigens, and where the one or more antigens comprises a norovirus NS7 protein, or fragment or epitope thereof.

The norovirus Pol protein (NS7) is an RNA dependent polymerase that plays the essential role of replicating the viral genome and is also generated by cleavage of the polyprotein encoded by ORF1 (where it is found between residues 1190 and 1699, using the canonical numbering system). As noted above, the ProPol precursor protein has been reported to share the protease activity of the released Pro protein; it has also been reported to have replicase activity of the released Pol protein (Belliot et al. J Virol. 77:10957, 2003, doi: 10.1128/JVI.77.20.10957-10974.2003; Belliot et al. J Virol. 79:2393, 2005, each of which is incorporated herein by reference in its entirety).

Phylogenetic comparisons of Pol protein sequences have been used to classify human noroviruses into sixty (60) different P types and P groups: fourteen (14) GI P types, thirty-seven (37) GII P types, two (2) GIII P types, two (2) GIV P types, two (2) GVI P types, one (1) GVII P types, one (1) GX P type, two tentative P groups, and fourteen (14) tentative P types (Chhabra et al. J Gen Virol. 100:1393, 2019, doi: 10.1099/jgv.0.001318, which is incorporated herein by reference in its entirety).

### Antigen Formats

In some embodiments, a an antigen utilized as described herein is or comprises a full-length viral protein. In some embodiments, an antigen utilized as described herein is or comprises a fragment or domain of a viral protein, or an antigenic portion thereof. In some embodiments, an antigen utilized as described herein is a membrane-tethered antigen (e.g., an antigenic fragment thereof fused with a membrane-associating moiety, such as for example, a transmembrane moiety). In some embodiments, a provided pharmaceutical composition (e.g., immunogenic composition, e.g., vaccine) comprises or delivers antigen sequences that are or comprise one or more antibody epitopes and/or one or more CD4 epitopes.

In some embodiments, an antigen utilized as described herein includes one or more variant sequences relative to a relevant reference antigen. For example, in some embodiments, a protease cleavage site is removed or blocked; alternatively or additionally, in some embodiments, a terminally truncated antigen is utilized.

In some embodiments, an antigen utilized as described herein includes a multimerization element (e.g., a heterologous multimerization element).

In some embodiments, an antigen utilized as described herein includes a membrane association element (e.g., a heterologous membrane association element), such as a transmembrane domain.

In some embodiments, an antigen utilized as described herein includes a secretion signal (e.g., a heterologous secretion signal).

In some embodiments, utilized sequences may be longer (and, e.g., may therefore include more epitopes) than a viral protein found in nature.

In some embodiments, utilized sequences may be from a different strain or plurality of strains (e.g., as may be circulating in and/or otherwise relevant to a population to which a pharmaceutical composition (e.g., immunogenic composition, e.g., vaccine) is administered).

In some embodiments, an antigen utilized as described herein may include a plurality of epitopes (e.g., B-cell and/or T-cell epitopes) arranged in a non-natural configuration (e.g., in a string construct as described herein). In some embodiments, an antigen utilized as described herein may include a plurality of epitopes predicted or demonstrated to bind HLA alleles reflective of a population to which a pharmaceutical composition (e.g., immunogenic composition, e.g., vaccine) composition is to be administered as described herein.

In some embodiments, a provided pharmaceutical composition (e.g., immunogenic composition, e.g., vaccine) may comprise or deliver a plurality of antigens. one or more antigens that includes B cell epitopes and one or more antigens that includes T cell epitopes. In some embodiments, a provided pharmaceutical composition (e.g., immunogenic composition, e.g., vaccine) may comprise or deliver one antigen that includes both B cell and CD4 epitopes and a separate antigen that includes CD8 epitopes.

### Combinations of Antigens

As described herein, in some embodiments, provided technologies involve administration of a plurality of antigens to the same subject. In some embodiments, multiple antigens are administered at the same time (e.g., in a single dose). In some embodiments, different antigens may be administered at different times (for example in different doses—e.g., a prime dose vs a boost dose). In some embodiments, multiple antigens are administered via the same composition.

For clarity, a single “antigen” polypeptide may include multiple “epitopes”, which in turn may or may not be linked with one another in nature. For example, a single string construct antigen includes multiple epitopes, which may be from different parts of the same viral protein and/or from different viral proteins, linked together as described herein in a single polypeptide.

Thus, a single pharmaceutical composition (e.g., immunogenic composition, e.g., vaccine) as described herein may comprise or deliver (e.g., because the pharmaceutical composition includes a nucleic acid, such as an RNA, that encodes the antigen and is expressed upon administration) a single antigen, which itself may comprise multiple epitopes (either in their natural arrangement relative to one another or in an engineered or constructed arrangement as described herein), or may comprise or deliver a plurality of antigens, each of which similarly may be or comprise a single epitope or multiple epitopes (either in their natural arrangement relative to one another or in an engineered or constructed arrangement as described herein). Still further, a single pharmaceutical composition (e.g., immunogenic composition, e.g., vaccine) may, for example, include multiple distinct nucleic acids (e.g., RNAs) that each encode different antigen(s) or, in some embodiments, may include a single nucleic acid that encodes (and expresses) multiple antigens. Yet further, a single pharmaceutical composition (e.g., immunogenic composition, e.g., vaccine) that includes multiple distinct nucleic acids (e.g., RNAs) encoding antigens may, in some embodiments be prepared by mixing the RNAs and then incorporating the mixture into LNPs, or alternatively by formulating individual RNAs into LNPs and then mixing the LNPs. In some embodiments, mixtures (whether of RNAs pre-LNP preparation or of LNPs) may include the relevant RNAs in 1:1 ratio, or in other ratios as may be preferred (e.g., to achieve a desired relative presentation of antigens or epitopes) in a subject to whom the composition is administered.

In some embodiments, a provided composition includes or delivers a viral antigen (e.g., a full-length viral protein, a fragment thereof, or one or more epitopes thereof, for example in a string construct). In some embodiments, a provided composition includes or delivers such a viral antigen together with one or more B cell targets (e.g., epitopes) which may, for example, be or comprise one or more other viral proteins (or fragments or epitopes thereof). In some embodiments, such a B cell target is or comprises a viral protein (or fragment or epitope thereof) that is predicted or known to induce a B cell response in infected humans.

In some embodiments, a provided composition comprises or delivers a string construct antigen that includes a plurality of T cell epitopes, optionally from more than one viral protein, in combination with a second viral antigen. In some such embodiments, a provided composition further comprises or delivers one or more B cell targets. Alternatively or additionally, in some embodiments, a string construct antigen so utilized includes viral sequences (e.g., one or more fragments or epitopes, e.g., T cell epitopes and/or B cell epitopes, but in some embodiments specifically T cell epitopes); in some such embodiments, a further viral antigen is also included or delivered.

In some embodiments, a string construct antigen includes both B cell epitopes and T cell epitopes (optionally from the same viral protein or from different viral proteins).

In some embodiments, different antigens may be delivered by administration of different compositions, which in turn may, in some embodiments, be administered at the same time (e.g., as an admixture or otherwise substantially simultaneously) and, in some embodiments, may be administered at different times. To give but one example, in some embodiments, a particular antigen or antigen(s) may be delivered via an initial pharmaceutical composition (e.g., immunogenic composition, e.g., vaccine) dose, and one or more other antigen(s) may be delivered via one or more booster dose(s).

In some embodiments, a construct comprises a sequence, or is encoded by a sequence, that is 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of the sequences in Tables 3A-3B, Tables 4A-4B, Tables 5A-5B, Tables 8A-8B, Tables 9A-9B, Tables 10A-10B, Tables 14A-14N, Tables 15A-15B, and Tables 16A-16P.

### Multi-Epitope Antigens

In some embodiments, an antigen utilized (i.e., included in and/or otherwise delivered by) a pharmaceutical composition (e.g., immunogenic composition, e.g., vaccine) described herein comprises multiple epitopes, e.g., of a single viral protein or of multiple proteins.

In some embodiments, an antigen may comprise two or more epitopes from the same viral protein and in their natural configuration relative to one another (e.g., in a fragment if the relevant protein). In some embodiments, however, an antigen may comprise at least two epitopes configured in a non-natural relationship relative to one another (e.g., included in a string construct as described herein.

Among other things, the present disclosure provides an insight that string construct antigens may be particularly useful or effective for vaccination against viral infection. Without wishing to be bound by any particular theory, the present disclosure proposes that ability to link individual epitopes predicted or determined to have specific attributes—e.g., binding to relevant HLA alleles, expression at relevant times of infection, representation of particularly conserved sequences, potentially across a plurality of different viral proteins, may prove uniquely beneficial, or indeed critical, for effective vaccination against viral, where more traditional vaccination approaches have thus far provided only limited protection.

In some embodiments, a multi-epitope antigen (e.g., a string construct antigen) may be administered as a polypeptide and/or as a collection of peptides. Alternatively or additionally, a multi-epitope antigen may be administered as preparation of cells that comprise (e.g., express) the antigen. However, the present disclosure further provides an insight that, in some embodiments, delivery by administration of a nucleic acid, and particularly of an RNA, encoding the multi-epitope antigen, may be particularly useful and/or effective.

As noted elsewhere herein, experience other viral vaccines has demonstrated that RNA administration can be a particularly effective way to deliver an infectious disease antigen. Furthermore, the present disclosure provides an insight that various features of nucleic acid formats including, for example their flexibility and amenability to rapid design and modification, including incorporation of a variety of insights (e.g., bioinformatics inputs etc), renders them particularly attractive for use in a viral vaccine. Among other things, the present disclosure provides an insight that, in some embodiments, administration of an RNA encoding a string antigen as described herein may be a particularly desirable and/or effective approach to immunizing against viral infection.

In some embodiments, a “string” polynucleotide sequence encodes a plurality of epitopes in tandem. In some embodiments, there are about 2 to about 100, about 2 to about 1000 or about 2 to about 10,000 epitopes encoded in one string. In some embodiments about 2-5,000 epitopes are encoded in one polynucleotide string. In some embodiments, about 2-4,000 epitopes are encoded in one polynucleotide string. In some embodiments, about 2-3,000 epitopes are encoded in one polynucleotide string. In some embodiments about 2-2,000 epitopes are encoded in one polynucleotide string. In some embodiments, about 2-1,000 epitopes are encoded in one polynucleotide string. In some embodiments, about 10-500 epitopes are encoded in one polynucleotide string. In some embodiments, about 10-200 epitopes are encoded in one polynucleotide string. In some embodiments, about 20-100 epitopes are encoded in one polynucleotide string.

In some embodiments, epitopes encoded by string constructs comprise epitopes that are predicted by a HLA binding and presentation prediction software to be of high likelihood to be presented by a protein encoded by an HLA to a T cell for eliciting immune response. In some embodiments, epitopes that are predicted to have a high likelihood to be presented by a protein encoded by an HLA, are selected from any one of the VZV proteins or peptides described in Table 4A and/or Tables 5A-5B. In some embodiments, epitopes that are predicted to have a high likelihood to be presented by a protein encoded by an HLA, are selected from any one of the CMV proteins or peptides described in Table 9A and/or Tables 10A-10B. In some embodiments, epitopes that are predicted to have a high likelihood to be presented by a protein encoded by an HLA, are selected from any one of the norovirus proteins or peptides described in Table 15A and/or Tables 16A-16P. In some embodiments, a string construct comprises one or more membrane, capsid, tegument, envelope associated, or otherwise accessible epitopes, e.g., at relevant time(s) during the viral life cycle.

In some embodiments, a string construct may comprise a multitude of epitopes that are from 2, 3, 4, or more viral proteins. In some embodiments, one or more string constructs may include one or more other epitopes (e.g., as may be predicted or demonstrated, for example in literature).

In some embodiments, a string construct may comprise sequences encoding features such as linkers, and cleavage sites (e.g., auto-cleavage sites such as, for example, T2A, or P2A sequences). In some embodiments, a linker that is enriched in G and S residues can be used. In some embodiments, an exemplary linker may have a sequence of GGGGSGGGGS or GGSGGGGSGG.

In some embodiments, a string construct comprises two or more overlapping epitope sequences.

In some embodiments, a string construct comprises a sequence, or is encoded by a sequence, that is 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of the sequences in Tables 3A-3B, Tables 4A-4B, Tables 5A-5B, Tables 8A-8B, Tables 9A-9B, Tables 10A-10B, Tables 14A-14N, Tables 15A-15B, and Tables 16A-16P. As noted above, where sequences being compared are longer than about 20 amino acids, percent identity or homology is typically greater than about 80%; for sequences longer than about 50 amino acids, percent identity or homology is typically greater than about 90%.

In some embodiments, epitopes are arranged on a string to maximize immunogenicity of the string, for example by maximizing recognition by HLA allele repertoire of a subject. In some embodiments, the same string encodes epitopes that can bind to and/or are predicted to bind to different HLA alleles. For instance, as is well exemplified in the sequences tables, e.g., at least in Tables 3A-3B, Tables 4A-4B, Tables 5A-5B, Tables 8A-8B, Tables 9A-9B, Tables 10A-10B, Tables 14A-14N, Tables 15A-15B, and/or Tables 16A-16P a string may encode epitope(s) that comprise: (a) a first epitope that binds to or is predicted to bind to a first MHC peptide encoded by a first HLA allele; (b) a second epitope that binds to or is predicted to bind to a second MHC peptide encoded by a second HLA allele; (c) a third epitope that binds to or is predicted to bind to a third MHC peptide encoded by a third HLA allele—and more such epitopes can be added, as in for example in string sequences as provided herein; wherein the first, second and third epitopes are epitopes from the same viral protein, or from different viral proteins. In this way, epitope distribution encoded by a single string is maximized for hitting the different MHC based presentation to T cells, thereby maximizing the probability of generating a desired immune response from a wider range of patients in the given population and the robustness of the response of each patient.

In some embodiments, epitopes included in a string construct are selected on the basis of high scoring prediction for binding to an HLA by a reliable prediction algorithm or system, such as the RECON prediction algorithm. In some embodiments, the present disclosure provides an insight that particularly successful strings can be provided by selecting epitopes based on highly reliable and efficient prediction algorithm, in the layout of the epitopes encoded by the string, with or without non-epitope sequences or sequences flanking the epitopes, and is such that the immunogenicity of the string is validated in an ex vivo cell culture model, or in an animal model, specifically in showing T cell induction following vaccination with a string construct or a polypeptide encoded by a string construct with the finding of epitope specific T cell response. In some embodiments, validation may be from using in human patients, and with a finding that T cells obtained from a patient post vaccination shows epitope specific efficient and lasting T cell response. In some embodiments, efficiency of a string as a vaccine is influenced by its design, that in part depends on strength of the bioinformatics information used in the thoughtful execution of the design, the reliability of the MHC presentation prediction model, the efficiency of epitope processing when a string vaccine is expressed in a cell, among others.

In some embodiments a multi-epitopic RNA (e.g., mRNA) construct as described above comprises 1, 2, 3, 4, 5, 6, 7, 8, 9, 10, 11, 12, 13, 14, 15 or more epitopes. In some embodiments, a pharmaceutical composition comprises 2, 3, 4, 5, 6, 7, 8, 9, or 10 or more strings. In some embodiments, a pharmaceutical composition comprises 6 strings. In some embodiments, a pharmaceutical composition comprises 7 strings. In some embodiments, a pharmaceutical composition comprises 8 strings. In some embodiments, a pharmaceutical composition comprises 9 strings. In some embodiments, a pharmaceutical composition comprises 10 strings.

In some embodiments, epitope-coding sequences in a string construct are flanked by one or more sequences selected for higher immunogenicity, better cleavability for peptide presentation to MHCs, better expression, and/or improved translation in a cell in a subject. In some embodiments, flanking sequences comprise a linker with a specific cleavable sequences. In some embodiments, epitope-coding sequences in a string construct are flanked by a secretory protein sequence.

In some embodiments, a string sequence encodes an epitope that may comprise or otherwise be linked to a signal sequence, such as those listed in Table 20, or at least a sequence having 1, 2, 3, 4, or 5 amino acid differences relative thereto. In some embodiments, a string sequence encodes an epitope that may comprise or otherwise be linked to a signal sequence such as MFVFLVLLPLVSSQCVNLT, or at least a sequence having 1, 2, 3, 4, or at the most 5 amino acid differences relative thereto. In some embodiments, a string sequence encodes an epitope that may be linked at the N-terminal end by a sequence that is enriched in G and S residues, or a sequence having 1, 2, 3, 4, or at the most 5 amino acid differences relative thereto. In some embodiments, an exemplary linker that may be useful to link epitopes has a sequence of GGSGGGGSGG.

In some embodiments, linked sequences may comprise a linker with a cleavage sequence, e.g., with specific cleavable sequences.

In some embodiments, a string construct is linked to a transmembrane domain (TM) or other membrane-associating element. In some embodiments, a linker may have a length of 2, 3, 4, 5, 6, 7, 8, 9, 10 or more amino acid. In some embodiments, a linker of not more than about 30, 25, 20, 15, 10 or fewer amino acids is used. In some embodiments, a linker sequence is not limited to comprise any particular amino acids; in some embodiments, a linker sequence comprises any amino acids. In some embodiments, a linker or cleavage sequence comprises glycine (G). In some embodiments, a linker or cleavage sequence comprises serine (S). In some embodiments, a linker is designed to comprise amino acids based on a cleavage predictor to generate highly-cleavable sequences peptide sequences, and is a novel and effective way of delivering immunogenic T cell epitopes in a T cell vaccine setting.

In some embodiments, epitope distribution and their juxtaposition encoded in a string construct are so designed to facilitate cleavage sequences contributed by the amino acid sequences of the epitopes and/or the flanking or linking residues and thereby using minimal linker sequences. Some exemplary cleavage sequences, without limitation, may be one or more of FRAC, KRCF, KKRY, ARMA, RRSG, MRAC, KMCG, ARCA, KKQG, YRSY, SFMN, FKAA, KRNG, YNSF, KKNG, RRRG, KRYS, and ARYA.

In some embodiments, a string construct is RNA (e.g., mRNA). In some embodiments, a pharmaceutical composition comprises one or more RNA (e.g., mRNA) string constructs, each comprising a sequence encoding a plurality of epitopes as described herein. In some embodiments, the one or more RNA (e.g., mRNA) comprises a plurality of epitopes, wherein each of the plurality of epitopes is predicted by an HLA binding and presentation prediction algorithm to be of high likelihood to be presented by a protein encoded by an HLA to a T cell for eliciting immune response.

In some embodiments, one or more RNAs (e.g., mRNAs) utilized in a pharmaceutical composition (e.g., immunogenic composition, e.g., vaccine) as described herein encodes a plurality of epitopes (e.g., one or more viral proteins as described herein), wherein each of the plurality of epitopes is predicted by an HLA binding and presentation prediction algorithm to be of high likelihood to be presented by a protein encoded by an HLA to a T cell for eliciting immune response. In some embodiments, the plurality of epitopes comprises epitopes from a single viral protein. In some embodiments, the plurality of epitopes comprises epitopes from multiple viral proteins.

In some embodiments, the RNA (e.g., mRNA) comprises a 5′UTR and a 3′UTR. In some embodiments, an UTR comprises a poly A sequence. In some embodiments, a poly A sequence comprises between 50-200 nucleotides.

In some embodiments, epitopes encoded in a string construct may be flanked by a signal peptide sequence, e.g., SP1 sequence (HSV-1 gD signal peptide/secretory domain).

In some embodiments, a polynucleotide comprises a dEarI-hAg sequence.

In some embodiments, a poly A tail of a string construct may comprise about 150 A residues. In some embodiments, a poly A tail may comprise 120 residues or less. In some embodiments, a poly A tail of a string construct may comprise about 120 A residues. In some embodiments, a poly A tail of a string construct may comprise about 100 residues. In some embodiments, a poly A tail of a string comprises a “split” or “interrupted” poly A tail (e.g., as described in WO2016/005324).

In some embodiments, a multi-epitope antigen encodes a super-motif-bearing or motif-bearing polypeptide, together with a helper epitope (e.g., a heterologous helper epitope) and an endoplasmic reticulum-translocating signal sequence. See, for example, in An & Whitton J. Virol. 71:2292, 1997; Thomson. et al., J. Immunol. 157:822, 1996; Whitton, et al., J. Virol 67:348, 1993; Hanke, et al., Vaccine 16:426, 1998.

### Multimerization Elements

In some embodiments, a viral antigen utilized as described herein includes a multimerization element (e.g., a heterologous multimerization element). In some embodiments, a heterologous multimerization element comprises a dimerization, trimerization or tetramerization element.

In some embodiments, a multimerization element is one described in WO2017/081082 (e.g., SEQ ID NOs: 1116-1167, or fragments or variants thereof).

Exemplary trimerization and tetramerization elements include, but are not limited to, engineered leucine zippers, fibritin foldon domain from enterobacteria phage T4, GCN4pll, GCN4-pll, and p53.

In some embodiments, a provided antigen is able to form a trimeric complex. For example, a utilized antigen may comprise a domain allowing formation of a multimeric complex, such as for example particular a trimeric complex of an amino acid sequence comprising a viral antigen as described herein. In some embodiments, a domain allowing formation of a multimeric complex comprises a trimerization domain, for example, a trimerization domain as described herein.

In some embodiments, a viral antigen can be modified by addition of a T4-fibritin-derived “foldon” trimerization domain, for example, to increase its immunogenicity.

### Membrane Association Elements

In some embodiments, a viral antigen as described herein includes a membrane association element (e.g., a heterologous membrane association element), such as a transmembrane domain.

A transmembrane domain can be N-terminal, C-terminal, or internal to an antigen. A coding sequence of a transmembrane element is typically placed in frame (i.e., in the same reading frame), 5′, 3′, or internal to coding sequences of sequences (e.g., viral antigen sequences) with which it is to be linked.

In some embodiments, a transmembrane domain comprises or is a transmembrane domain of HSV-1, Hemagglutinin (HA) of Influenza virus, Env of HIV-1, equine infectious anaemia virus (EIAV), murine leukaemia virus (MLV), mouse mammary tumor virus, G protein of vesicular stomatitis virus (VSV), Rabies virus, or a seven transmembrane domain receptor. In some embodiments, a transmembrane domain comprises or is a HSV-1 gD transmembrane domain. In some embodiments, an HSV-1 gD transmembrane domain comprises or consists of an amino acid sequence having at least 95% identity to the amino acid sequence of GLIAGAVGGSLLAALVICGIVYWMRRHTQKAPKRIRLPHIR. In some embodiments, an HSV-1 gD transmembrane domain comprises or consists of the amino acid sequence of GLIAGAVGGSLLAALVICGIVYWMRRHTQKAPKRIRLPHIR.

### Secretory Sequences

In some embodiments, a viral antigen for use in accordance with the present disclosure includes a secretory sequence, e.g., that is functional in mammalian cells.

In some embodiments, a utilized secretory sequence is “intrinsic” in that it is associated in the viral antigen with sequences with which it is normally associated in nature (e.g., in included in a viral protein, or fragment or epitope thereof, that is included in the utilized antigen).

In some embodiments, a utilized secretory sequence is heterologous to other sequences of the relevant antigen—e.g., is not naturally part of viral protein whose sequences are included in the antigen.

In some embodiments, signal peptides are sequences, which are typically characterized by a length of about 15 to 30 amino acids.

In many embodiments, signal peptides are positioned at the N-terminus of a viral antigen construct as described herein, without being limited thereto. In some embodiments, signal peptides preferably allow the transport of the viral antigen with which they are associated into a defined cellular compartment, preferably the cell surface, the endoplasmic reticulum (ER) or the endosomal-lysosomal compartment.

In some embodiments, a secretory sequence is selected from an S1S2 signal peptide (e.g., aa 1-19), an immunoglobulin secretory signal peptide (e.g., aa 1-22), an HSV-1 gD signal peptide (MGGAAARLGAVILFVVIVGLHGVRSKY), an HSV-2 gD signal peptide (MGRLTSGVGTAALLVVAVGLRVVCA); a human SPARC signal peptide, a human insulin isoform 1 signal peptide, a human albumin signal peptide, etc. Those skilled in the art will be aware of other secretory signal peptides such as, for example, as disclosed in WO2017/081082 (e.g., SEQ ID NOs: 1-1115 and 1728, or fragments variants thereof). In certain embodiments, a signal peptide is an IgG signal peptide, such as an IgG kappa signal peptide.

In some embodiments, a string sequence encodes an epitope that may comprise or otherwise be linked to a signal sequence (e.g., secretory sequence), such as those listed in Table 20, or at least a sequence having 1, 2, 3, 4, or 5 amino acid differences relative thereto. In some embodiments, a secretory sequence such as MFVFLVLLPLVSSQCVNLT, or at least a sequence having 1, 2, 3, 4, or at the most 5 amino acid differences relative thereto is utilized. In some embodiments, a sequence such as MFVFLVLLPLVSSQCVNLT,or a sequence having 1, 2, 3, 4, or at the most 5 amino acid differences relative thereto, is utilized.

In some embodiments, a signal sequence is selected from those included in the Table 20 below and/or those encoded by the sequences in Table 21 below:

TABLE 20
Exemplary signal sequences

| Signal | Sequence (Amino Acid) |
|---|---|
| HSV-1 gD SP | MGGAAARLGAVILFVVIVGLHGVRSKY |
| HSV-1 gD SP | MGGAAARLGAVILFVVIVGLHGVRGKY |
| HSV-2 gD SP | MGRLTSGVGTAALLVVAVGLRVVCA |
| HSV-2 | MGRLTSGVGTAALLVVAVGLRVVCAKYA |
| SARS-COV- | MFVFLVLLPLVSSQCVNLT |
| 2-S | |
| human Ig | MDWIWRILFLVGAATGAHSQM |
| heavy chain | |
| signal peptide | |
| (huSec) | |
| HuIgGk | METPAQLLFLLLLWLPDTTG |
| signal peptide | |
| IgE heavy | MDWTWILFLVAAATRVHS |
| chain epsilon- | |
| 1signal | |
| peptide | |
| Japanese | MLGSNSGQRVVFTILLLLVAPAYS |
| encephalitis | |
| PRM signal | |
| sequence | |
| VSVg protein | MKCLLYLAFLFIGVNCA |
| signal | |
| sequence | |
| human Ig | MELGLSWIFLLAILKGVQC |
| heavy chain | |
| signal peptide | |
| human Ig | MELGLRWVFLVAILEGVQC |
| heavy chain | |
| signal peptide | |
| human Ig | MKHLWFFLLLVAAPRWVLS |
| heavy chain | |
| signal peptide | |
| human Ig | MDWTWRILFLVAAATGAHS |
| heavy chain | |
| signal peptide | |
| human Ig | MDWTWRFLFVVAAATGVQS |
| heavy chain | |
| signal peptide | |
| human Ig | MEFGLSWLFLVAILKGVQC |
| heavy chain | |
| signal peptide | |
| human Ig | MEFGLSWVFLVALFRGVQC |
| heavy chain | |
| signal peptide | |
| human Ig | MDLLHKNMKHLWFFLLLVAAPRWVLS |
| heavy chain | |
| signal peptide | |
| human Ig | MDMRVPAQLLGLLLLWLSGARC |
| kappa chain | |
| signal peptide | |
| human Ig | MKYLLPTAAAGLLLLAAQPAMA |
| kappa chain | |
| signal peptide | |

TABLE 21
Exemplary nucleotide sequences encoding signal sequences

| Signal | Sequence (Nucleotide) |
|---|---|
| HSV-1 gD SP | ATGGGGGGGGCTGCCGCCAGGTTGGGGGCCGTGATTTT |
| wild-type | GTTTGTCGTCATAGTGGGCCTCCATGGGGTCCGCAGCA |
| | AATAT |
| HSV-1 gD SP | ATGGGAGGAGCCGCCGCCAGACTGGGAGCCGTGATCCT |
| optimized | GTTCGTGGTGATCGTGGGACTGCATGGAGTGAGAAGCA |
| nucleotide | AGTAC |
| sequence | |
| SARS-COV- | ATGTTTGTGTTTCTTGTGCTGCTGCCTCTTGTGTCTTCTC |
| 2-S | AGTGTGTGAATTTGACA |
| human Ig | ATGGATTGGATTTGGAGAATCCTGTTCCTCGTGGGAGC |
| heavy chain | CGCTACAGGAGCCCACTCCCAGATG |
| signal peptide | |
| (huSec) | |
| human Ig | ATGGAGTTGGGACTGAGCTGGATTTTCCTTTTGGCTATT |
| heavy chain | TTAAAAGGTGTCCAGTGT |
| signal peptide | |
| human Ig | ATGGAACTGGGGCTCCGCTGGGTTTTCCTTGTTGCTATT |
| heavy chain | TTAGAAGGTGTCCAGTGT |
| signal peptide | |
| human Ig | ATGAAACACCTGTGGTTCTTCCTCCTGCTGGTGGCAGCT |
| heavy chain | CCCAGATGGGTCCTGTCC |
| signal peptide | |
| human Ig | ATGGACTGGACCTGGAGGATCCTCTTCTTGGTGGCAGC |
| heavy chain | AGCAACAGGTGCCCACTCG |
| signal peptide | |
| human Ig | ATGGACTGGACCTGGAGGTTCCTCTTTGTGGTGGCAGC |
| heavy chain | AGCTACAGGTGTCCAGTCC |
| signal peptide | |
| human Ig | ATGGAGTTTGGGCTGAGCTGGCTTTTTCTTGTGGCGATT |
| heavy chain | CTAAAAGGTGTCCAGTGT |
| signal peptide | |
| human Ig | ATGGAGTTTGGGCTGAGCTGGGTTTTCCTCGTTGCTCTT |
| heavy chain | TTTAGAGGTGTCCAGTGT |
| signal peptide | |
| human Ig | ATGGACCTCCTGCACAAGAACATGAAACACCTGTGGTT |
| heavy chain | CTTCCTCCTCCTGGTGGCAGCTCCCAGATGGGTGCTGTC |
| signal peptide | C |
| human Ig | ATGGACATGAGGGTCCCTGCTCAGCTCCTGGGGCTCCT |
| kappa chain | GCTGCTCTGGCTCTCAGGTGCCAGATGT |
| signal peptide | |
| human Ig | ATGAAATACCTATTGCCTACGGCAGCCGCTGGATTGTT |
| kappa chain | ATTACTCGCGGCCCAGCCGGCCATGGCC |
| signal peptide | |

### Linkers

In some embodiments, a linker may have a length of 2, 3, 4, 5, 6, 7, 8, 9, 10 or more amino acid. In some embodiments, a linker of not more than about 30, 25, 20, 15, 10 or fewer amino acids is used. In some embodiments, a linker sequence is not limited to comprise any particular amino acids; in some embodiments, a linker sequence comprises any amino acids. In some embodiments, a linker or cleavage sequence comprises glycine (G). In some embodiments, a linker or cleavage sequence comprises serine (S). In some embodiments, a linker is designed to comprise amino acids based on a cleavage predictor to generate highly-cleavable sequences peptide sequences, and is a novel and effective way of delivering immunogenic T cell epitopes in a T cell vaccine setting.

In some embodiments, a linker is or comprises S-G4-S-G4-S. In some embodiments, a linker is or comprises GSPGSGSGS. In some embodiments, a linker is or comprises GGSGGGGSGG. In some embodiments, a linker is selected from those presented in Table 22 below:

TABLE 22
Exemplary linkers

| Linker | Sequence (Amino Acid) |
|---|---|
| GSS | SGGGGSGGGGS |
| GSP | GSPGSGSGS |
| GS | GSGSGS |
| GGD | GGSGGGGSGG |
| From | GGSLGGGGSG |
| GGSL | |

In some embodiments, a linker is or comprises a sequence as set forth in WO2017/081082 (see SEQ ID NOs: 1509-1565), or a fragment or variant thereof.

### Helper Epitopes

Those skilled in the art are aware that effective B cell responses often require assistance from helper T cells. As noted herein, it is proposed that an effective viral vaccine, particularly if it targets viral protein(s) (e.g., envelope protein(s), tegument protein(s), membrane protein(s), or combination thereof) expressed prior to cellular infection, may benefit from or require ability to induce particularly robust antibody response. In some embodiments, it may be desirable for a provided pharmaceutical composition (e.g., immunogenic composition, e.g., vaccine) to include or deliver one or more “helper epitopes” (i.e., CD4 T cell epitopes), for example in addition to one or more B-cell epitopes (and/or one or more CD8 T cell embodiments).

The present disclosure proposes that helper epitope(s) may be particularly useful when a pharmaceutical composition (e.g., immunogenic composition, e.g., vaccine) includes or delivers an antigen that includes repeated elements. In some embodiments, a provided pharmaceutical composition (e.g., immunogenic composition, e.g., vaccine) does not include or deliver an antigen that includes such repeated elements. Regardless, in some embodiments, a provided pharmaceutical composition (e.g., immunogenic composition, e.g., vaccine) does not include or deliver any helper epitope(s), or at least not helper epitope(s) (e.g., heterologous helper element(s)) specifically engineered into an antigen.

Where helper epitope(s) are desired, those skilled in the art are aware of a variety of potentially useful sequences, including those for example discussed in WO2020128031 which include, for instance, helper epitope(s) derived from P2 tetanus toxin, PADRE helper epitope, Hepatitis B surface antigen (HBsAg).

### RNAs

In many embodiments, provided pharmaceutical compositions (e.g., immunogenic compositions, e.g., vaccines) deliver antigens as described herein by delivering a nucleic acid construct, e.g., in many embodiments, an RNA construct, that encodes one or more antigens as described herein and is expressed in the subject upon administration of the pharmaceutical composition (e.g., immunogenic composition, e.g., vaccine).

Among other things, the present disclosure encompasses the recognition that administration of nucleic acid, and particularly of RNA to achieve delivery (e.g., by expression) of encoded antigen can provide a variety of benefits relative to other strategies for immunizing against viral infection.

Among other things, the present disclosure provides an insight that RNA may be particularly useful and/or effective as an active agent in pharmaceutical compositions (e.g., immunogenic compositions, e.g., viral vaccines for a variety of reasons including specifically that RNA can have intrinsic adjuvanticity. As noted herein, ability to induce very high antibody titers to viral proteins, e.g., particularly those expressed and/or targeted prior to cellular infiltration.

Still further, experience with SARS-CoV-2 vaccines has demonstrated that RNA actives can also elicit significant and diverse T cell responses which, particularly when combined with strong antibody response, represents a combination of immune characteristics thought to potentially maximize the probability of protection.

### Payload

In many embodiments, an RNA provided by the present disclosure (e.g., as may be included in a pharmaceutical composition (e.g., immunogenic composition, e.g., vaccine) as described herein or otherwise used to produce one or more viral antigens as described herein) includes a “payload” element that encodes a polypeptide of interest (e.g., a viral antigen as described herein).

### Codon Optimization and GC Enrichment

In some embodiments, a payload element is codon optimized for expression in the subject to whom a pharmaceutical composition (e.g., immunogenic composition, e.g., vaccine) is to be administered (e.g., a human). Thus, in many embodiments, sequences in such a polynucleotide may differ from wild type sequences encoding the relevant antigen or fragment or epitope thereof, even when the amino acid sequence of the antigen or fragment or epitope thereof is wild type.

Those skilled in the art are familiar with strategies for codon optimization for expression in a relevant host (e.g., a human host), and even, in some cases, for expression in a particular cell or tissue.

In general, as is understood, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of a native sequence with codons that are more frequently or most frequently used in the genes of that host cell while maintaining the native amino acid sequence.

Various species exhibit particular bias for certain codons of a particular amino acid. Without wishing to be bound by any one theory, codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell may generally be a reflection of the codons used most frequently in peptide synthesis. Accordingly, genes may be tailored for optimal gene expression in a given organism based on codon optimization. Codon usage tables are available, for example, at the “Codon Usage Database” available at www.kazusa.orjp/codon/and these tables may be adapted in a number of ways. Computer algorithms for codon optimizing a particular sequence for expression in a particular host cell are also available, such as Gene Forge (Aptagen; Jacobus, PA), are also available.

In some embodiments, a polynucleotide of the present disclosure is codon optimized, wherein the codons in a polynucleotide are adapted to human codon usage (herein referred to as “human codon optimized polynucleotide”). Codons encoding the same amino acid occur at different frequencies in a subject, e.g., a human. Accordingly, in some embodiments, the coding sequence of a polynucleotide of the present disclosure is modified such that the frequency of the codons encoding the same amino acid corresponds to the naturally occurring frequency of that codon according to the human codon usage, e.g., as shown in Table 23. For example, in the case of the amino acid Ala, the wild type coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with 30 a frequency of 0.10 etc. (see Table 23). Accordingly, in some embodiments, such a procedure (as exemplified for Ala) is applied for each amino acid encoded by the coding sequence of a polynucleotide to obtain sequences adapted to human codon usage.

TABLE 23
Human codon usage table with frequencies indicated for each amino acid

| Amino acid | codon | frequency | Amino acid | codon | frequency |
|---|---|---|---|---|---|
| Ala | GCG | 0.10 | Pro | CCG | 0.11 |
| Ala | GCA | 0.22 | Pro | CCA | 0.27 |
| Ala | GCT | 0.28 | Pro | CCT | 0.29 |
| Ala | GCC* | 0.40 | Pro | CCC* | 0.33 |
| Cys | TGT | 0.42 | Gln | CAG* | 0.73 |
| Cys | TGC* | 0.58 | Gln | CAA | 0.27 |
| Asp | GAT | 0.44 | Arg | AGG | 0.22 |
| Asp | GAC* | 0.56 | Arg | AGA* | 0.21 |
| Glu | GAG* | 0.59 | Arg | CGG | 0.19 |
| Glu | GAA | 0.41 | Arg | CGA | 0.10 |
| Phe | TTT | 0.43 | Arg | CGT | 0.09 |
| Phe | TTC* | 0.57 | Arg | CGC | 0.19 |
| Gly | GGG | 0.23 | Ser | AGT | 0.14 |
| Gly | GGA | 0.26 | Ser | AGC* | 0.25 |
| Gly | GGT | 0.18 | Ser | TCG | 0.06 |
| Gly | GGC* | 0.33 | Ser | TCA | 0.15 |
| His | CAT | 0.41 | Ser | TCT | 0.18 |
| His | CAC* | 0.59 | Ser | TCC | 0.23 |
| Ile | ATA | 0.14 | Thr | ACG | 0.12 |
| Ile | ATT | 0.35 | Thr | ACA | 0.27 |
| Ile | ATC* | 0.52 | Thr | ACT | 0.23 |
| Lys | AAG* | 0.60 | Thr | ACC* | 0.38 |
| Lys | AAA | 0.40 | Val | GTG* | 0.48 |
| Leu | TTG | 0.12 | Val | GTA | 0.10 |
| Leu | TTA | 0.06 | Val | GTT | 0.17 |
| Leu | CTG* | 0.43 | Val | GTC | 0.25 |
| Leu | CTA | 0.07 | Trp | TGG* | 1 |
| Leu | CTT | 0.12 | Tyr | TAT | 0.42 |
| Leu | CTC | 0.20 | Tyr | TAC* | 0.58 |
| Met | ATG* | 1 | Stop | TGA* | 0.61 |
| Asn | AAT | 0.44 | Stop | TAG | 0.17 |
| Asn | AAC* | 0.56 | Stop | TAA | 0.22 |

Certain strategies for codon optimization and/or G/C enrichment for human expression are described in WO2002/098443. In some embodiments, a coding sequence may be optimized using a multiparametric optimization strategy. In some embodiments optimization parameters may include parameters that influence protein expression, which can be, for example, impacted on a transcription level, an mRNA level, and/or a translational level. In some embodiments, exemplary optimization parameters include, but are not limited to transcription-level parameters (including, e.g., GC content, consensus splice sites, cryptic splice sites, SD sequences, TATA boxes, termination signals, artificial recombination sites, and combinations thereof); mRNA-level parameters (including, e.g., RNA instability motifs, ribosomal entry sites, repetitive sequences, and combinations thereof); translation-level parameters (including, e.g., codon usage, premature poly(A) sites, ribosomal entry sites, secondary structures, and combinations thereof); or combinations thereof. In some embodiments, a coding sequence may be optimized by a GeneOptimizer algorithm as described in Fath et al. “Multiparameter RNA and Codon Optimization: A Standardized Tool to Assess and Enhance Autologous Mammalian Gene Expression” PLoS ONE 6(3): e17596; Rabb et al., “The GeneOptimizer Algorithm: using a sliding window approach to cope with the vast sequence space in multiparameter DNA sequence optimization” Systems and Synthetic Biology (2010) 4:215-225; and Graft et al. “Codon-optimized genes that enable increased heterologous expression in mammalian cells and elicit efficient immune responses in mice after vaccination of naked DNA” Methods Mol Med (2004) 94:197-210, the entire content of each of which is incorporated herein for the purposes described herein. In some embodiments, a coding sequence may be optimized by Eurofins' adaption and optimization algorithm “GENEius” as described in Eurofins' Application Notes: Eurofins' adaption and optimization software “GENEius” in comparison to other optimization algorithms, the entire content of which is incorporated by reference for the purposes described herein.

In some embodiments, a payload coding sequence utilized in accordance with the present disclosure has G/C content of which increased compared to a wild type coding sequence for a relevant viral antigen, or fragment or epitope thereof. In some embodiments, guanosine/cytidine (G/C) content of a payload coding region is modified relative to a wild type coding sequence for a relevant viral antigen, or fragment or epitope thereof, but the amino acid sequence encoded by the RNA not modified.

Without wishing to be bound by any particular theory, it is proposed that GC enrichment may improve translation of a payload sequence. Typically, sequences having an increased G (guanosine)/C (cytidine) content are more stable than sequences having an increased A (adenosine)/U (uridine) content. In respect to the fact that several codons code for one and the same amino acid (so-called degeneration of the genetic code), the most favorable codons for the stability can be determined (so-called alternative codon usage). Depending on the amino acid to be encoded by the RNA, there are various possibilities for modification of the RNA sequence, compared to its wild type sequence. In particular, codons which contain A and/or U nucleosides can be modified by substituting these codons by other codons, which code for the same amino acids but contain no A and/or U or contain a lower content of A and/or U nucleosides.

In some embodiments, G/C content of a payload coding region of an RNA described herein is increased by at least 10%, at least 20%, at least 30%, at least 40%, at least 50%, at least 55%, or even more.

In some embodiments, stability and translation efficiency of an RNA may incorporate one or more elements established to contribute to stability and/or translation efficiency of RNA; exemplary such elements are described, for example, in PCT/EP2006/009448 incorporated herein by reference. In some embodiments, to increase expression of an RNA used according to the present disclosure, an RNA may be modified within the coding region, i.e. the sequence encoding the expressed peptide or protein, without altering the sequence of the expressed peptide or protein, for example so as to increase the GC-content to increase mRNA stability and/or to perform a codon optimization and, thus, enhance translation in cells.

### RNA Formats

At least three distinct formats useful for RNA compositions (e.g., pharmaceutical compositions) have been developed, namely non-modified uridine containing mRNA (uRNA), nucleoside-modified mRNA (modRNA), and self-amplifying mRNA (saRNA). Each of these platforms displays unique features. In general, in all three formats, RNA is capped, contains open reading frames (ORFs) flanked by untranslated regions (UTR), and have a polyA-tail at the 3′ end. An ORF of an uRNA and modRNA vectors encode an antibody agent or portion thereof. An saRNA has multiple ORFs.

In some embodiments, the RNA described herein may have modified nucleosides. In some embodiments, the RNA comprises a modified nucleoside in place of at least one (e.g. every) uridine.

The term “uracil,” as used herein, describes one of the nucleobases that can occur in the nucleic acid of RNA. The structure of uracil is:

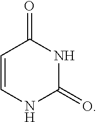

The term “uridine,” as used herein, describes one of the nucleosides that can occur in RNA. The structure of uridine is:

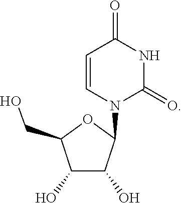 [SMILES: O=c1ccn([C@@H]2O[C@H](CO)[C@@H](O)[C@H]2O)c(=O)[nH]1]

UTP (uridine 5′-triphosphate) has the following structure:

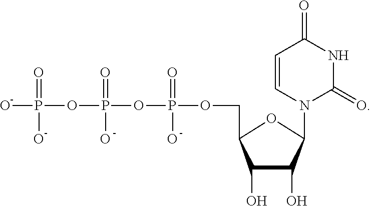

Pseudo-UTP (pseudouridine 5′-triphosphate) has the following structure:

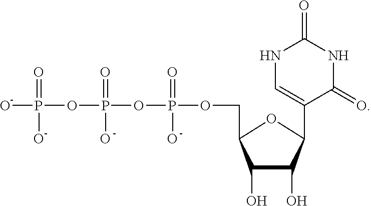

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1Ψ), which has the structure:

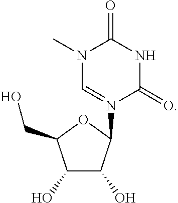

N1-methyl-pseudo-UTP has the following structure:

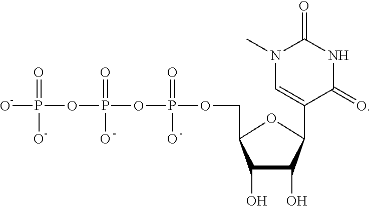

Another exemplary modified nucleoside is 5-methyl-uridine (m5U), which has the structure:

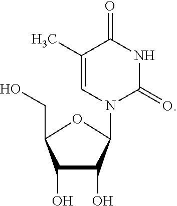

In some embodiments, one or more uridine in the RNA described herein is replaced by a modified nucleoside. In some embodiments, the modified nucleoside is a modified uridine.

In some embodiments, RNA comprises a modified nucleoside in place of at least one uridine. In some embodiments, RNA comprises a modified nucleoside in place of each uridine.

In some embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, the modified nucleoside replacing one or more, e.g., all, uridine in the RNA may be any one or more of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine(τm5s2U), 1-taurinomethyl-4-thio-pseudouridine), 5-methyl-2-thio-uridine (m5s2U), 1-methyl-4-thio-pseudouridine (m1sψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-0-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In some embodiments, the RNA comprises other modified nucleosides or comprises further modified nucleosides, e.g., modified cytidine. For example, in some embodiments, in the RNA 5-methylcytidine is substituted partially or completely, preferably completely, for cytidine. In some embodiments, the RNA comprises 5-methylcytidine and one or more selected from pseudouridine (ψ), N1-methyl-pseudouridine (m1ψ), and 5-methyl-uridine (m5U). In some embodiments, the RNA comprises 5-methylcytidine and N1-methyl-pseudouridine (m1ψ). In some embodiments, the RNA comprises 5-methylcytidine in place of each cytidine and N1-methyl-pseudouridine (m1ψ) in place of each uridine.

In some embodiments of the present disclosure, the RNA is “replicon RNA” or simply a “replicon,” in particular “self-replicating RNA” or “self-amplifying RNA.” In one particularly preferred embodiment, the replicon or self-replicating RNA is derived from or comprises elements derived from a single-stranded (ss) RNA virus, in particular a positive-stranded ssRNA virus, such as an alphavirus. Alphaviruses are typical representatives of positive-stranded RNA viruses. Alphaviruses replicate in the cytoplasm of infected cells (for review of the alphaviral life cycle see Jose et al., Future Microbiol., 2009, vol. 4, pp. 837-856, which is incorporated herein by reference in its entirety). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234).

Alphavirus-derived vectors have been proposed for delivery of foreign genetic information into target cells or target organisms. In simple approaches, a first ORF encodes an alphavirus-derived RNA-dependent RNA polymerase (replicase), which upon translation mediates self-amplification of the RNA. A second ORF encoding alphaviral structural proteins is replaced by an open reading frame encoding a protein of interest, e.g., an antibody agent. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

Features of a non-modified uridine platform may include, for example, one or more of intrinsic adjuvant effect, as well as good tolerability and safety. Features of modified uridine (e.g., pseudouridine) platform may include reduced adjuvant effect, blunted immune innate immune sensor activating capacity and thus good tolerability and safety. Features of self-amplifying platform may include, for example, long duration of protein expression, good tolerability and safety, higher likelihood for efficacy with very low vaccine dose.

The present disclosure provides particular RNA constructs optimized, for example, for improved manufacturability, encapsulation, expression level (and/or timing), etc. Certain components are discussed below, and certain preferred embodiments are exemplified herein.

### 5′ Cap

A structural feature of mRNAs is cap structure at five-prime end (5′). Natural eukaryotic mRNA comprises a 7-methylguanosine cap linked to the mRNA via a 5′ to 5′-triphosphate bridge resulting in cap0 structure (m7GpppN). In most eukaryotic mRNA and some viral mRNA, further modifications can occur at the 2′-hydroxy-group (2′-OH) (e.g., the 2′-hydroxyl group may be methylated to form 2′-O-Me) of the first and subsequent nucleotides producing “cap1” and “cap2” five-prime ends, respectively). Diamond, et al., (2014) Cytokine & growth Factor Reviews, 25:543-550 reported that cap0-mRNA cannot be translated as efficiently as cap1-mRNA in which the role of 2′-O-Me in the penultimate position at the mRNA 5′ end is determinant. Lack of the 2′-O-met has been shown to trigger innate immunity and activate IFN response. Daffis, et al. (2010) Nature, 468:452-456; and Züst et al. (2011) Nature Immunology, 12:137-143.

RNA capping is well researched and is described, e.g., in Decroly E et al. (2012) Nature Reviews 10: 51-65; and in Ramanathan A. et al., (2016) Nucleic Acids Res; 44(16): 7511-7526, the entire contents of each of which is hereby incorporated by reference. For example, in some embodiments, a 5′-cap structure which may be suitable in the context of the present invention is a cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (“anti-reverse cap analogue”), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

The term “5′-cap” as used herein refers to a structure found on the 5′-end of an RNA, e.g., mRNA, and generally includes a guanosine nucleotide connected to an RNA, e.g., mRNA, via a 5′- to 5′-triphosphate linkage (also referred to as Gppp or G(5′)ppp(5′)). In some embodiments, a guanosine nucleoside included in a 5′ cap may be modified, for example, by methylation at one or more positions (e.g., at the 7-position) on a base (guanine), and/or by methylation at one or more positions of a ribose. In some embodiments, a guanosine nucleoside included in a 5′ cap comprises a 3′O methylation at a ribose (3′OMeG). In some embodiments, a guanosine nucleoside included in a 5′ cap comprises methylation at the 7-position of guanine (m7G). In some embodiments, a guanosine nucleoside included in a 5′ cap comprises methylation at the 7-position of guanine and a 3′ O methylation at a ribose (m7(3′OMeG)). It will be understood that the notation used in the above paragraph, e.g., “(m₂^{7,3′-O})G” or “m7(3′OMeG)”, applies to other structures described herein.

In some embodiments, providing an RNA with a 5′-cap disclosed herein may be achieved by in vitro transcription, in which a 5′-cap is co-transcriptionally expressed into an RNA strand, or may be attached to an RNA post-transcriptionally using capping enzymes. In some embodiments, co-transcriptional capping with a cap disclosed improves the capping efficiency of an RNA compared to co-transcriptional capping with an appropriate reference comparator. In some embodiments, improving capping efficiency can increase a translation efficiency and/or translation rate of an RNA, and/or increase expression of an encoded polypeptide. In some embodiments, alterations to polynucleotides generates a non-hydrolyzable cap structure which can, for example, prevent decapping and increase RNA half-life.

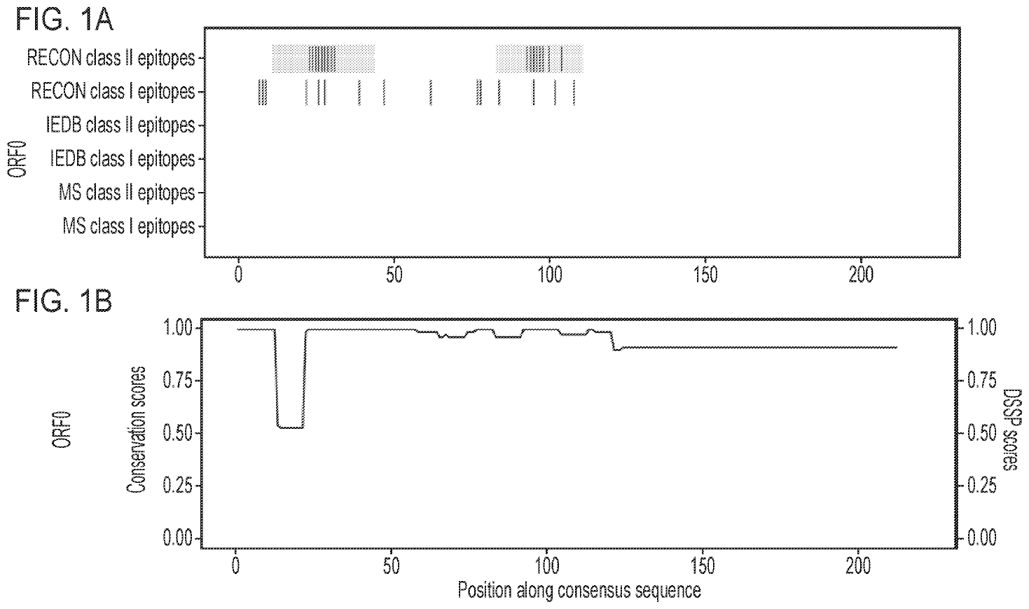

In some embodiments, a utilized 5′ caps is a cap0, a cap1, or cap2 structure. See, e.g., FIG. 1 of Ramanathan A et al., and FIG. 1 of Decroly E et al., each of which is incorporated herein by reference in its entirety. See, e.g., FIG. 1 of Ramanathan A et al., and FIG. 1 of Decroly E et al., each of which is incorporated herein by reference in its entirety. In some embodiments, an RNA described herein comprises a cap1 structure. In some embodiments, an RNA described herein comprises a cap2.

In some embodiments, an RNA described herein comprises a cap0 structure. In some embodiments, a cap0 structure comprises a guanosine nucleoside methylated at the 7-position of guanine ((m⁷)G). In some embodiments, such a cap0 structure is connected to an RNA via a 5′- to 5′-triphosphate linkage and is also referred to herein as (m⁷)Gppp. In some embodiments, a cap0 structure comprises a guanosine nucleoside methylated at the 2′-position of the ribose of guanosine. In some embodiments, a cap0 structure comprises a guanosine nucleoside methylated at the 3′-position of the ribose of guanosine. In some embodiments, a guanosine nucleoside included in a 5′ cap comprises methylation at the 7-position of guanine and at the 2′-position of the ribose ((m₂^{7,2′-O})G). In some embodiments, a guanosine nucleoside included in a 5′ cap comprises methylation at the 7-position of guanine and at the 2′-position of the ribose ((m₂^{7,3′-O})G).

In some embodiments, a cap1 structure comprises a guanosine nucleoside methylated at the 7-position of guanine ((m⁷)G) and optionally methylated at the 2′ or 3′ position pf the ribose, and a 2′O methylated first nucleotide in an RNA ((m^{2′-O})N₁). In some embodiments, a cap1 structure comprises a guanosine nucleoside methylated at the 7-position of guanine ((m⁷)G) and the 3′ position of the ribose, and a 2′O methylated first nucleotide in an RNA ((m^{2′-O})N₁). In some embodiments, a cap1 structure is connected to an RNA via a 5′- to 5′-triphosphate linkage and is also referred to herein as, e.g., ((m⁷)Gppp(^{2′-O})N₁) or (m₂^{7,3′-O})Gppp(^{2′-O})N₁), wherein N₁ is as defined and described herein. In some embodiments, a cap1 structure comprises a second nucleotide, N₂, which is at position 2 and is chosen from A, G, C, or U, e.g., (m⁷)Gppp(^{2′-O})N₁pN₂ or (m₂^{7,3′-O})Gppp(^{2′-O})N₁pN₂, wherein each of N₁ and N₂ is as defined and described herein.

In some embodiments, a cap2 structure comprises a guanosine nucleoside methylated at the 7-position of guanine ((m⁷)G) and optionally methylated at the 2′ or 3′ position pf the ribose, and a 2′O methylated first and second nucleotides in an RNA ((m^{2′-O})N₁p(m^{2′-O})N₂). In some embodiments, a cap2 structure comprises a guanosine nucleoside methylated at the 7-position of guanine ((m⁷)G) and the 3′ position of the ribose, and a 2′O methylated first and second nucleotide in an RNA. In some embodiments, a cap2 structure is connected to an RNA via a 5′- to 5′-triphosphate linkage and is also referred to herein as, e.g., ((m⁷)Gppp(^{2′-O})N₁p(^{2′-O})N₂) or (m₂^{7,3′-O})Gppp(^{2′-O})N₁p(^{2′-O})N₂), wherein each of N₁ and N₂ is as defined and described herein.

In some embodiments, the 5′ cap is a dinucleotide cap structure. In some embodiments, the 5′ cap is a dinucleotide cap structure comprising N₁, wherein N₁ is as defined and described herein. In some embodiments, the 5′ cap is a dinucleotide cap G*N₁, wherein N₁ is as defined above and herein, and G* comprises a structure of formula (I):

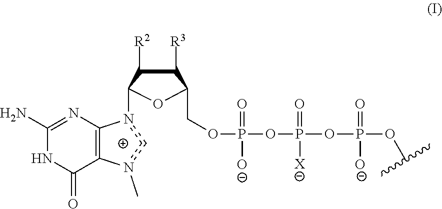

or a salt thereof,
wherein
   each R² and R³ is —OH or —OCH₃; and
   X is 0 or S.

In some embodiments, R² is —OH. In some embodiments, R² is —OCH₃. In some embodiments, R³ is —OH. In some embodiments, R³ is —OCH₃. In some embodiments, R² is —OH and R³ is —OH. In some embodiments, R² is —OH and R³ is —CH₃. In some embodiments, R² is —CH₃ and R³ is —OH. In some embodiments, R² is —CH₃ and R³ is —CH₃.

In some embodiments, X is O. In some embodiments, X is S.

In some embodiments, the 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O})GppSpN₁, or (m₂^{7,3′-O})GppSpN₁), wherein N₁ is as defined and described herein. In some embodiments, the 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O})GppSpN₁, or (m₂^{7,3′-O})GppSpN₁), wherein N₁ is G. In some embodiments, the 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O})GppSpN₁, or (m₂^{7,3′-O})GppSpN₁), wherein N₁ is A, U, or C. In some embodiments, the 5′ cap is a dinucleotide cap1 structure (e.g., (m⁷)Gppp(m^{2′-O})N₁, (m₂^{7,2′-O})Gppp(m^{2′-O})N₁, (m₂^{7,3′-O})Gppp(m^{2′-O})N₁, (m⁷)GppSp(m^{2′-O})N₁, (m₂^{7,2′-O})GppSp(m^{2′-O})N₁, or (m₂^{7,3′-O})GppSp(m^{2′-O})N₁), wherein N₁ is as defined and described herein. In some embodiments, the 5′ cap is selected from the group consisting of (m⁷)GpppG (“Ecap0”), (m⁷)Gppp(m^{2′-O})G (“Ecap1”), (m₂^{7,3′-O})GpppG (“ARCA” or “D1”), and (m₂^{7,2′-O})GppSpG (“beta-S-ARCA”). In some embodiments, the 5′ cap is (m⁷)GpppG (“Ecap0”), having a structure:

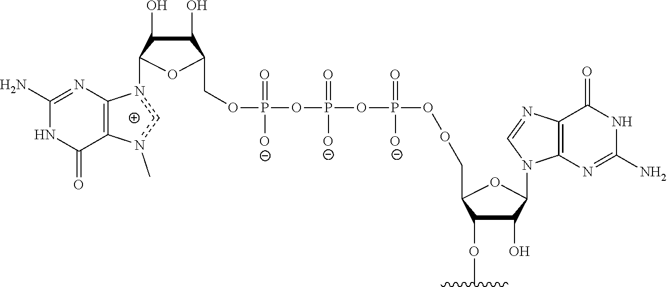

or a salt thereof.

In some embodiments, the 5′ cap is (m⁷)Gppp(m^{2′-O})G (“Ecap1”), having a structure:

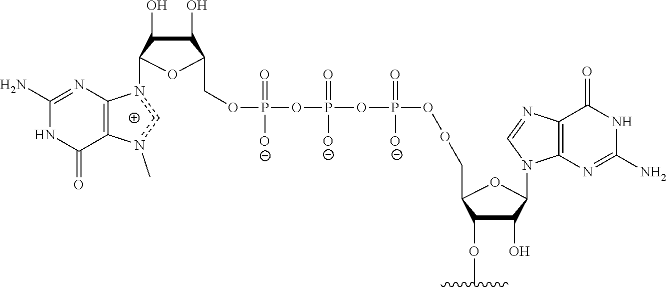

or a salt thereof.

In some embodiments, the 5′ cap is (m₂^{7,3′-O})GpppG (“ARCA” or “D1”), having a structure:

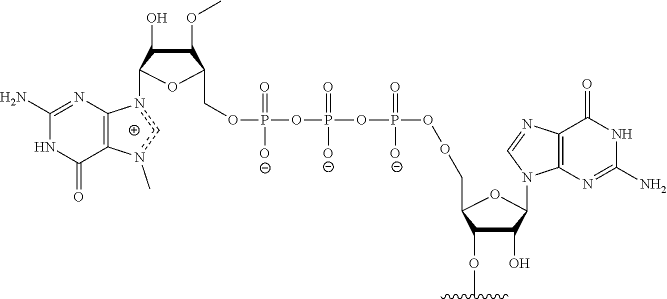

or a salt thereof.

In some embodiments, the 5′ cap is (m₂^{7,2′-O})GppSpG (“beta-S-ARCA”), having a structure:

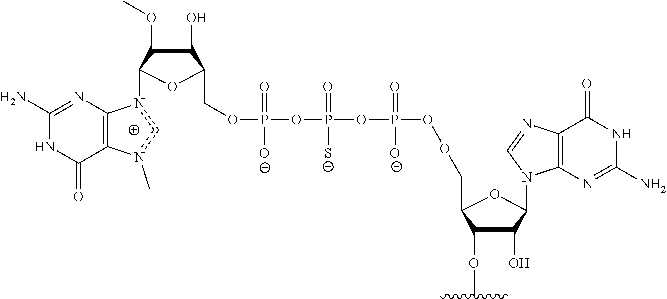

or a salt thereof.

In some embodiments, the 5′ cap is a trinucleotide cap structure. In some embodiments, the 5′ cap is a trinucleotide cap structure comprising N₁pN₂, wherein N₁ and N₂ are as defined and described herein. In some embodiments, the 5′ cap is a dinucleotide cap G*N₁pN₂, wherein N₁ and N₂ are as defined above and herein, and G* comprises a structure of formula (I):

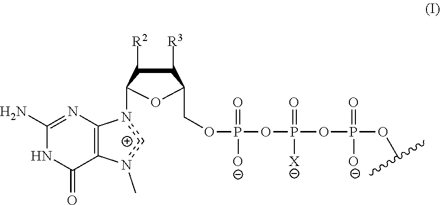

or a salt thereof, wherein R², R³, and X are as defined and described herein.

In some embodiments, the 5′ cap is a trinucleotide cap0 structure (e.g. (m⁷)GpppN₁pN₂, (m₂^{7,2′-O})GpppN₁pN₂, or (2^{7,3′-O})GpppN₁pN₂), wherein N₁ and N₂ are as defined and described herein). In some embodiments, the 5′ cap is a trinucleotide cap1 structure (e.g., (m⁷)Gppp(m^{2′-O})N₁pN₂, (m₂^{7,2′-O})Gppp(m^{2′-O})N₁pN₂, (m₂^{7,3′-O})Gppp(m^{2′-O})N₁pN₂), wherein N₁ and N₂ are as defined and described herein. In some embodiments, the 5′ cap is a trinucleotide cap2 structure (e.g., (m⁷)Gppp(m^{2′-O})N₁p(m^{2′-O})N₂, (m₂^{7,2′-O})Gppp(m^{2′-O})N₁p(m^{2′-O})N₂, (m₂^{7,3′-O})Gppp(m^{2′-O})N₁p(m^{2′-O})N₂), wherein N₁ and N₂ are as defined and described herein. In some embodiments, the 5′ cap is selected from the group consisting of (m₂^{7,3′-O})Gppp(m^{2′-O})ApG (“CleanCap AG”, “CC413”), (m₂^{7,3′-O}Gppp(m₂^{7,2′-O})GpG (“CleanCap GG”), (m⁷)Gppp(m^{2′-O})ApG, (m⁷)Gppp(m^{2′-O})GpG, (m₂^{7,3′-O})Gppp(m₂^{6,2′-O})ApG, and (m⁷)Gppp(m^{2′-O})ApU.

In some embodiments, the 5′ cap is (m₂^{7,3′-O})Gppp(m^{2′-O})ApG (“CleanCap AG”, “CC413”), having a structure:

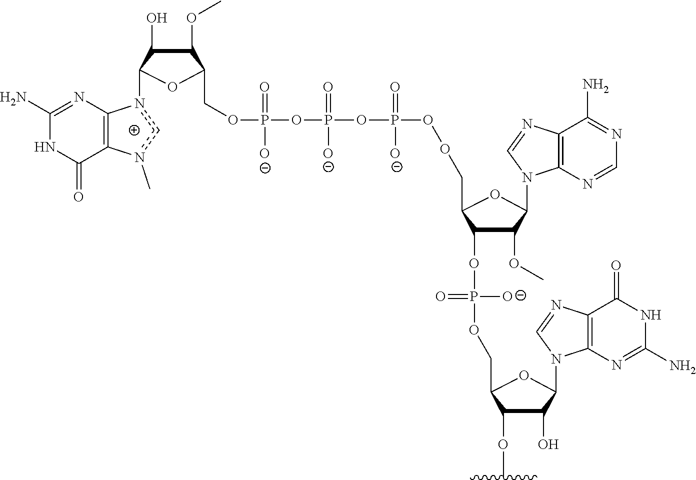

or a salt thereof.

In some embodiments, the 5′ cap is (m₂^{7,3′-O})Gppp(m^{2′-O})GpG (“CleanCap GG”), having a structure:

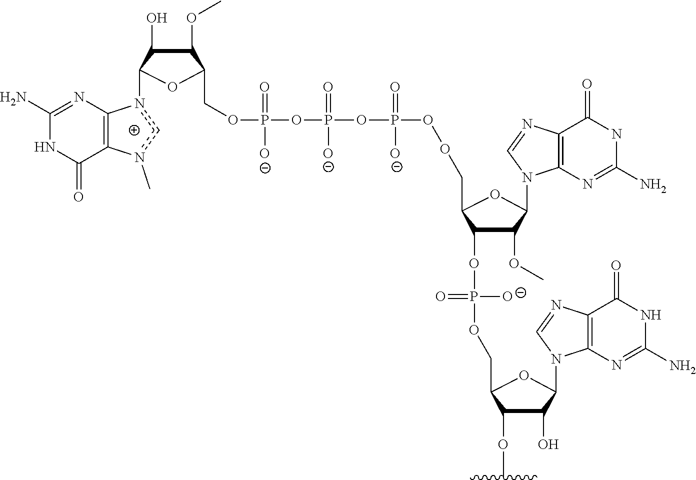

or a salt thereof.

In some embodiments, the 5′ cap is (m⁷)Gppp(m^{2′-O})ApG, having a structure:

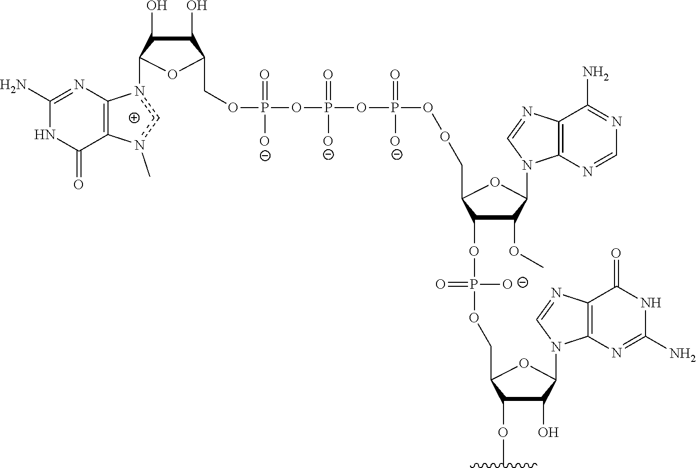

or a salt thereof.

In some embodiments, the 5′ cap is (m⁷)Gppp(m^{2′-O})GpG, having a structure:

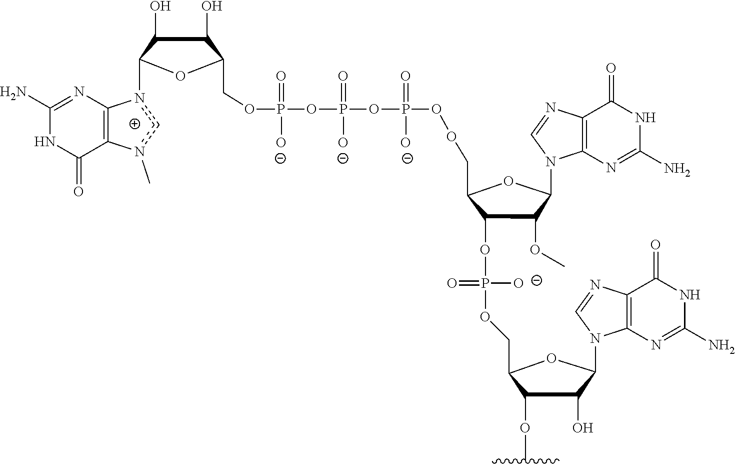

or a salt thereof.

In some embodiments, the 5′ cap is (m₂^{7,3′-O})Gppp(m₂^{6,2′-O})ApG, having a structure:

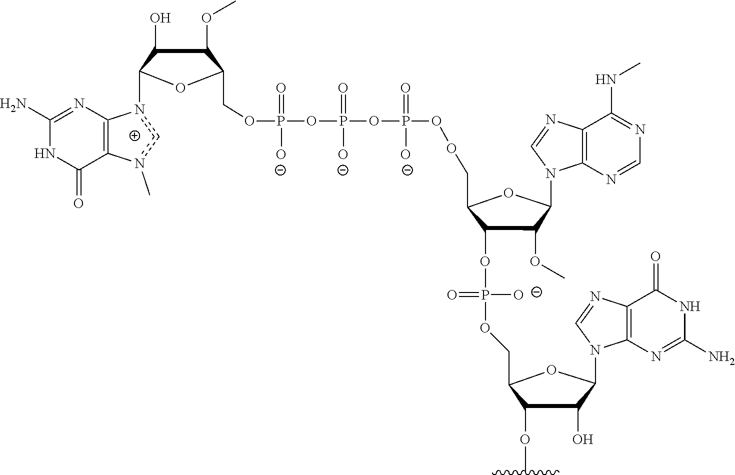

or a salt thereof.

In some embodiments, the 5′ cap is (m⁷)Gppp(m^{2′-O})ApU, having a structure:

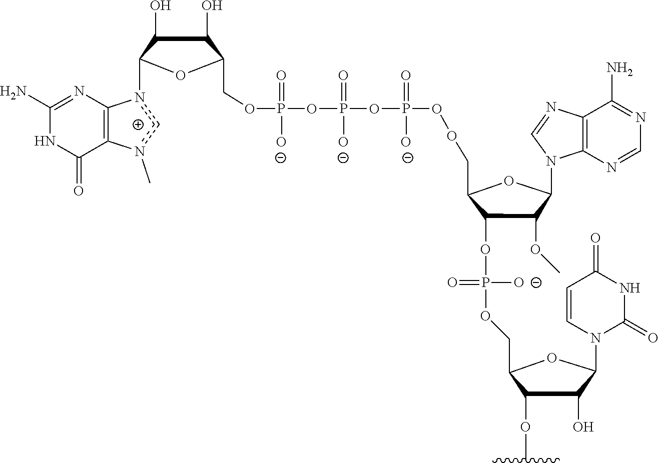

or a salt thereof.

In some embodiments, the 5′ cap is a tetranucleotide cap structure. In some embodiments, the 5′ cap is a tetranucleotide cap structure comprising N₁pN₂pN₃, wherein N₁, N₂, and N₃ are as defined and described herein. In some embodiments, the 5′ cap is a tetranucleotide cap G*N₁pN₂pN₃, wherein N₁, N₂, and N₃ are as defined above and herein, and G* comprises a structure of formula (I):

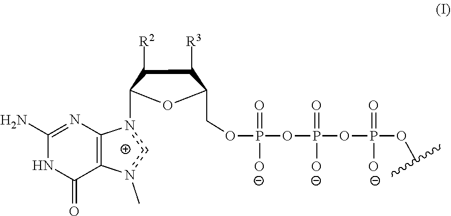

or a salt thereof, wherein R², R³, and X are as defined and described herein.

In some embodiments, the 5′ cap is a tetranucleotide cap0 structure (e.g. (m⁷)GpppN₁pN₂pN₃, (m₂^{7,2′-O})GpppN₁pN₂pN₃, or (m₂^{7,3′-O})GpppN₁N₂pN₃), wherein N₁, N₂, and N₃ are as defined and described herein). In some embodiments, the 5′ cap is a tetranucleotide Cap1 structure (e.g., (m⁷)Gppp(m^{2′-O})N₁pN₂pN₃, (m₂^{7,2′-O})Gppp(m^{2′-O})N₁pN₂pN₃, (m₂^{7,3′-O})Gppp(m^{2′-O})N₁pN₂N₃), wherein N₁, N₂, and N₃ are as defined and described herein. In some embodiments, the 5′ cap is a tetranucleotide Cap2 structure (e.g., (m⁷)Gppp(m^{2′-O})N₁p(m^{2′-O})N₂pN₃, (m₂^{7,3′-O})Gppp(m^{2′-O})N₁p(m^{2′-O})N₂pN₃, (m₂^{7,3′-O})Gppp(m^{2′-O})N₁p(m^{2′-O})N₂pN₃), wherein N₁, N₂, and N₃ are as defined and described herein. In some embodiments, the 5′ cap is selected from the group consisting of (m₂^{7,3′-O})Gppp(m^{2′-O})Ap(m^{2′-O})GpG, (m₂^{7,3′-O})Gppp(m^{2′-O})Gp(m^{2′-O})GpC, (m⁷)Gppp(m^{2′-O})Ap(m^{2′-O})UpA, and (m⁷)Gppp(m^{2′-O})Ap(m^{2′-O})GpG.

In some embodiments, the 5′ cap is (m₂^{7,3′-O})Gppp(m^{2′-O})Ap(m^{2′-O})GpG, having a structure:

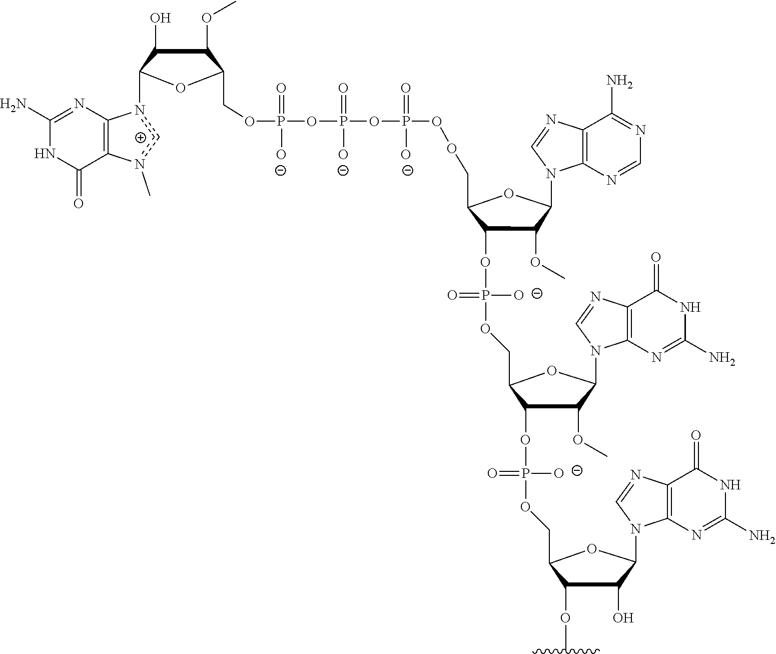

or a salt thereof.

In some embodiments, the 5′ cap is (m₂^{7,3′-O})Gppp(m^{2′-O})Gp(m^{2′-O})GpC, having a structure:

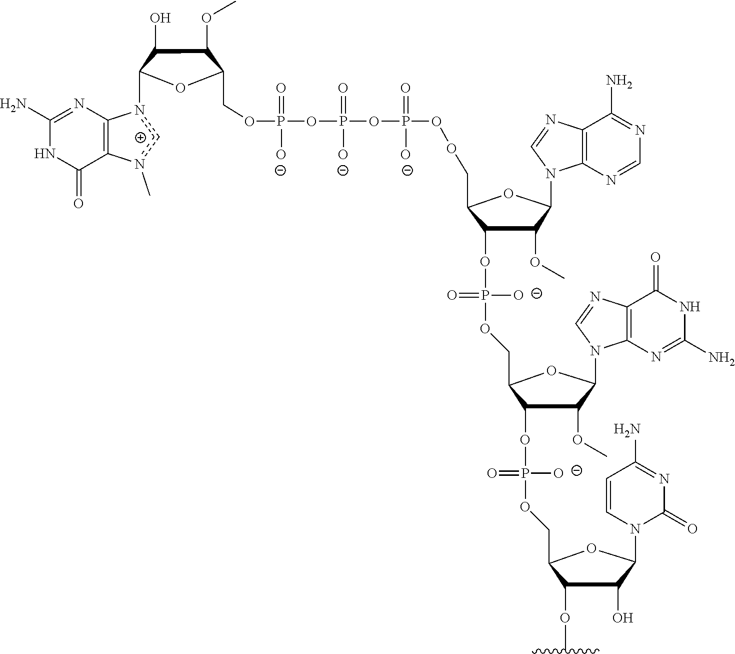

or a salt thereof.

In some embodiments, the 5′ cap is (m⁷)Gppp(m^{2′-O})Ap(m^{2′-O})UpA, having a structure:

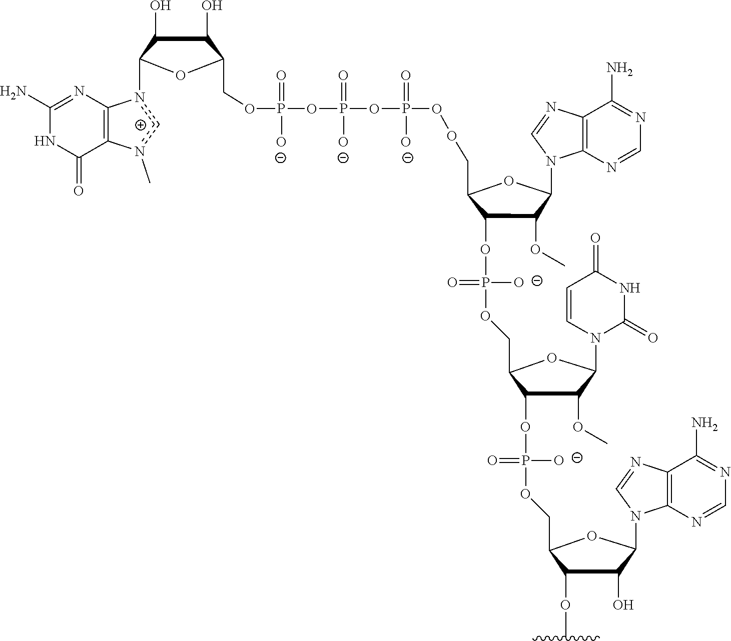

or a salt thereof.

In some embodiments, the 5′ cap is (m⁷)Gppp(m^{2′-O})Ap(m^{2′-O})GpG, having a structure:

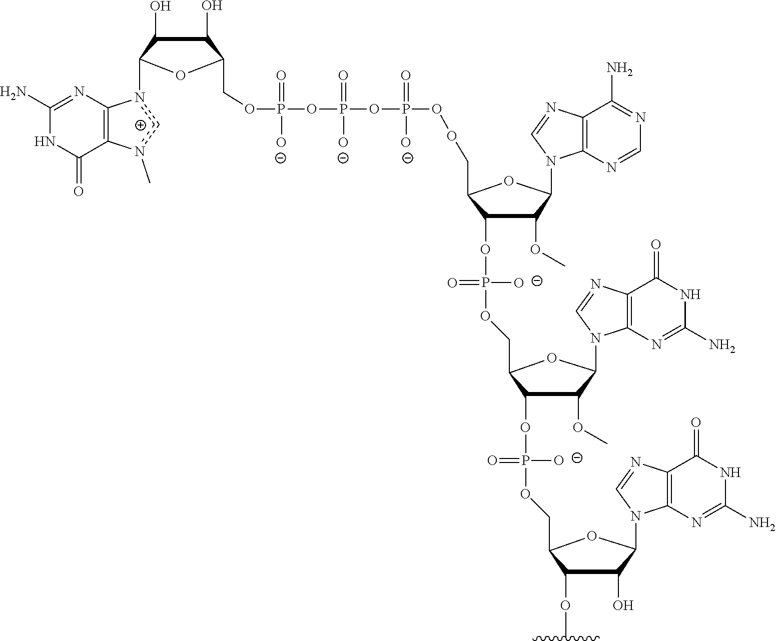

or a salt thereof.

### Cap Proximal Sequences

In some embodiments, a 5′ UTR utilized in accordance with the present disclosure comprises a cap proximal sequence, e.g., as disclosed herein. In some embodiments, a cap proximal sequence comprises a sequence adjacent to a 5′ cap. In some embodiments, a cap proximal sequence comprises nucleotides in positions +1, +2, +3, +4, and/or +5 of an RNA polynucleotide.

In some embodiments, a cap structure comprises one or more polynucleotides of a cap proximal sequence. In some embodiments, a cap structure comprises an m⁷ Guanosine cap and nucleotide+1 (N₁) of an RNA polynucleotide. In some embodiments, a cap structure comprises an m⁷ Guanosine cap and nucleotide+2 (N₂) of an RNA polynucleotide. In some embodiments, a cap structure comprises an m⁷ Guanosine cap and nucleotides+1 and +2 (N₁ and N₂) of an RNA polynucleotide. In some embodiments, a cap structure comprises an m⁷ Guanosine cap and nucleotides+1, +2, and +3 (N₁, N₂, and N₃) of an RNA polynucleotide.

Those skilled in the art, reading the present disclosure, will appreciate that, in some embodiments, one or more residues of a cap proximal sequence (e.g., one or more of residues+1, +2, +3, +4, and/or +5) may be included in an RNA by virtue of having been included in a cap entity (e.g., a cap1 or cap2 structure, etc); alternatively, in some embodiments, at least some of the residues in a cap proximal sequence may be enzymatically added (e.g., by a polymerase such as a T7 polymerase). For example, in certain exemplified embodiments where a m₂^{7,3′-O}Gppp(m₁^{2′-O})ApG cap is utilized, +1 (i.e., N₁) and +2 (i.e. N₂) are the (m₁^{2′-O})A and G residues of the cap, and +3, +4, and +5 are added by polymerase (e.g., T7 polymerase).

In some embodiments, the 5′ cap is a dinucleotide cap structure, wherein the cap proximal sequence comprises N₁ of the 5′ cap, where N₁ is any nucleotide, e.g., A, C, G or U. In some embodiments, the 5′ cap is a trinucleotide cap structure (e.g., the trinucleotide cap structures described above and herein), wherein the cap proximal sequence comprises N₁ and N₂ of the 5′ cap, wherein N₁ and N₂ are independently any nucleotide, e.g., A, C, G or U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁, N₂, and N₃ are any nucleotide, e.g., A, C, G or U.

In some embodiments, e.g., where the 5′ cap is a dinucleotide cap structure, a cap proximal sequence comprises N₁ of a the 5′ cap, and N₂, N₃, N₄ and N₅, wherein N₁ to N₅ correspond to positions +1, +2, +3, +4, and/or +5 of an RNA polynucleotide. In some embodiments, e.g., where the 5′ cap is a trinucleotide cap structure, a cap proximal sequence comprises N₁ and N₂ of a the 5′ cap, and N₃, N₄ and N₅, wherein N₁ to N₅ correspond to positions +1, +2, +3, +4, and/or +5 of an RNA polynucleotide. In some embodiments, e.g., where the 5′ cap is a tetranucleotide cap structure, a cap proximal sequence comprises N₁, N₂, and N₃ of a the 5′ cap, and N₄ and N₅, wherein N₁ to N₅ correspond to positions +1, +2, +3, +4, and/or +5 of an RNA polynucleotide.

In some embodiments, N₁ is A. In some embodiments, N₁ is C. In some embodiments, N₁ is G. In some embodiments, N₁ is U. In some embodiments, N₂ is A. In some embodiments, N₂ is C. In some embodiments, N₂ is G. In some embodiments, N₂ is U. In some embodiments, N₃ is A. In some embodiments, N₃ is C. In some embodiments, N₃ is G. In some embodiments, N₃ is U. In some embodiments, N₄ is A. In some embodiments, N₄ is C. In some embodiments, N₄ is G. In some embodiments, N₄ is U. In some embodiments, N₅ is A. In some embodiments, N₅ is C. In some embodiments, N₅ is G. In some embodiments, N₅ is U. It will be understood that, each of the embodiments described above and herein (e.g., for N₁ through N₅) may be taken singly or in combination and/or may be combined with other embodiments of variables described above and herein (e.g., 5′ caps).

### 5′ UTR

In some embodiments, a nucleic acid (e.g., DNA, RNA) utilized in accordance with the present disclosure a 5′-UTR. In some embodiments, 5′-UTR may comprise a plurality of distinct sequence elements; in some embodiments, such plurality may be or comprise multiple copies of one or more particular sequence elements (e.g., as may be from a particular source or otherwise known as a functional or characteristic sequence element). In some embodiments a 5′ UTR comprises multiple different sequence elements.

The term “untranslated region” or “UTR” is commonly used in the art to a region in a DNA molecule which is transcribed but is not translated into an amino acid sequence, or to the corresponding region in an RNA polynucleotide, such as an mRNA molecule. An untranslated region (UTR) can be present 5′ (upstream) of an open reading frame (5′-UTR) and/or 3′ (downstream) of an open reading frame (3′-UTR). As used herein, the terms “five prime untranslated region” or “5′ UTR” refer to a sequence of a polyribonucleotide between the 5′ end of the polyribonucleotide (e.g., a transcription start site) and a start codon of a coding region of the polyribonucleotide. In some embodiments, “5′ UTR” refers to a sequence of a polyribonucleotide that begins at the 5′ end of the polyribonucleotide (e.g., a transcription start site) and ends one nucleotide (nt) before a start codon (usually AUG) of a coding region of the polyribonucleotide, e.g., in its natural context. In some embodiments, a 5′ UTR comprises a Kozak sequence. A 5′-UTR is downstream of the 5′-cap (if present), e.g., directly adjacent to the 5′-cap. In some embodiments, a 5′ UTR disclosed herein comprises a cap proximal sequence, e.g., as defined and described herein. In some embodiments, a cap proximal sequence comprises a sequence adjacent to a 5′ cap.

Exemplary 5′ UTRs include a human alpha globin (hAg) 5′UTR or a fragment thereof, a TEV 5′ UTR or a fragment thereof, a HSP70 5′ UTR or a fragment thereof, or a c-Jun 5′ UTR or a fragment thereof.

In some embodiments, an RNA disclosed herein comprises a hAg 5′ UTR or a fragment thereof.

In some embodiments, an RNA disclosed herein comprises a 5′ UTR having at least 80%, at least 85%, at least 90%, at least 95%, at least 96%, at least 97%, at least 98%, or at least 99% identity to a 5′ UTR with the sequence according to SEQ ID NO: 472. In some embodiments, an RNA disclosed herein comprises a 5′ UTR provided in SEQ ID NO: 472.

### PolyA

In some embodiments, a polynucleotide (e.g., DNA, RNA) disclosed herein comprises a polyadenylate (PolyA) sequence, e.g., as described herein. In some embodiments, a PolyA sequence is situated downstream of a 3′-UTR, e.g., adjacent to a 3′-UTR.

As used herein, the term “poly(A) sequence” or “poly-A tail” refers to an uninterrupted or interrupted sequence of adenylate residues which is typically located at the 3′-end of an RNA polynucleotide. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. In some embodiments, polynucleotides disclosed herein comprise an uninterrupted Poly(A) sequence. In some embodiments, polynucleotides disclosed herein comprise interrupted Poly(A) sequence. In some embodiments, RNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

It has been demonstrated that a poly(A) sequence of about 120 A nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017).

In some embodiments, a poly(A) sequence in accordance with the present disclosure is not limited to a particular length; in some embodiments, a poly(A) sequence is any length. In some embodiments, a poly(A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in accordance with the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. In some embodiments, the poly(A) sequence contained in an RNA polynucleotide described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly(A) sequence at its 3′-end, i.e., the poly(A) sequence is not masked or followed at its 3′-end by a nucleotide other than A.

In some embodiments, the poly(A) sequence may comprise at least 20, at least 30, at least 40, at least 80, or at least 100 and up to 500, up to 400, up to 300, up to 200, or up to 150 nucleotides. In some embodiments, the poly(A) sequence may essentially consist of at least 20, at least 30, at least 40, at least 80, or at least 100 and up to 500, up to 400, up to 300, up to 200, or up to 150 nucleotides. In some embodiments, the poly(A) sequence may consist of at least 20, at least 30, at least 40, at least 80, or at least 100 and up to 500, up to 400, up to 300, up to 200, or up to 150 nucleotides. In some embodiments, the poly(A) sequence comprises at least 100 nucleotides. In some embodiments, the poly(A) sequence comprises about 150 nucleotides. In some embodiments, the poly(A) sequence comprises about 120 nucleotides.

In some embodiments, a poly A tail comprises a specific number of Adenosines, such as about 50 or more, about 60 or more, about 70 or more, about 80 or more, about 90 or more, about 100 or more, about 120, or about 150 or about 200. In some embodiments a poly A tail of a string construct may comprise 200 A residues or less. In some embodiments, a poly A tail of a string construct may comprise about 200 A residues. In some embodiments, a poly A tail of a string construct may comprise 180 A residues or less. In some embodiments, a poly A tail of a string construct may comprise about 180 A residues. In some embodiments, a poly A tail may comprise 150 residues or less.

### 3′ UTR

In some embodiments, an RNA utilized in accordance with the present disclosure comprises a 3′-UTR.

A 3′-UTR, if present, is located at the 3′ end, downstream of the termination codon of a protein-encoding region, but the term “3′-UTR” does preferably not include the poly(A) sequence. Thus, the 3′-UTR is upstream of the poly(A) sequence (if present), e.g. directly adjacent to the poly(A) sequence.

In some embodiments, an RNA disclosed herein comprises a 3′ UTR comprising an F element and/or an I element. In some embodiments, a 3′ UTR or a proximal sequence thereto comprises a restriction site. In some embodiments, a restriction site is a BamHI site. In some embodiments, a restriction site is a XhoI site.

In some embodiments, an RNA construct comprises an F element. In some embodiments, a F element sequence is a 3′-UTR of amino-terminal enhancer of split (AES).

In some embodiments, an RNA disclosed herein comprises a 3′ UTR having 99%, 98%, 97%, 96%, 95%, 90%, 85%, or 80% identity to a 3′ UTR with the sequence comprising: CUGGUACUGCAUGCACGCAAUGCUAGCUGCCCCUUUCCCGUCCUGGGUACCCC GAGUCUCCCCCGACCUCGGGUCCCAGGUAUGCUCCCACCUCCACCUGCCCCAC UCACCACCUCUGCUAGUUCCAGACACCUCCCAAGCACGCAGCAAUGCAGCUCA AAACGCUUAGCCUAGCCACACCCCCACGGGAAACAGCAGUGAUUAACCUUUA GCAAUAAACGAAAGUUUAACUAAGCUAUACUAACCCCAGGGUUGGUCAAUUU CGUGCCAGCCACACC (SEQ ID NO: A). In some embodiments, an RNA disclosed herein comprises a 3′ UTR provided in SEQ ID NO: A.

In some embodiments, a 3′UTR is an FI element as described in WO2017/060314.

### Exemplary RNA Configuration

Among other things, the present disclosure provides a polyribonucleotide encoding a polypeptide that comprises an HSV-1 gD secretory signal and/or an HSV-1 gD transmembrane domain with one or more viral antigens. The present disclosure provides the insight that such a polyribonucleotide will result in a polypeptide that can be effectively presented to a subject's immune system and induce an immune response. Accordingly, the present disclosure provides the recognition that such polypeptides, and encoding polyribonucleotides, are particularly effective in pharmaceutical compositions (e.g., immunogenic compositions, e.g., vaccines) that will be administered a subject (e.g., a human).

In some embodiments, a polyribonucleotide can encode a polypeptide, where the polypeptide comprises an HSV-1 gD secretory signal, one or more viral antigens, and an HSV-1 gD transmembrane domain. In some embodiments, a method can comprise administering a polyribonucleotide encoding a polypeptide to a subject, where the polypeptide comprises an HSV-1 gD secretory signal, one or more viral antigens, and an HSV-1 gD transmembrane domain, and where the subject is a mammal (e.g., a human).

In some embodiments, an HSV-1 gD secretory signal comprises or consists of an amino acid sequence having at least 95% identity to the amino acid sequence of MGGAAARLGAVILFVVIVGLHGVRSKY. In some embodiments, an HSV-1 gD secretory signal comprises or consists of the amino acid sequence of MGGAAARLGAVILFVVIVGLHGVRSKY. In some embodiments, an HSV-1 gD secretory signal comprises or consists of an amino acid sequence having at least 95% identity to the amino acid sequence of MGGAAARLGAVILFVVIVGLHGVRGKY. In some embodiments, an HSV-1 gD secretory signal comprises or consists of the amino acid sequence of MGGAAARLGAVILFVVIVGLHGVRGKY.

In some embodiments, an HSV-1 gD transmembrane domain comprises or consists of an amino acid sequence having at least 95% identity to the amino acid sequence of GLIAGAVGGSLLAALVICGIVYWMRRHTQKAPKRIRLPHIR. In some embodiments, an HSV-1 gD transmembrane domain comprises or consists of the amino acid sequence of GLIAGAVGGSLLAALVICGIVYWMRRHTQKAPKRIRLPHIR.

In some embodiments, one or more viral antigens can be from any viral pathogen. In some embodiments, one or more viral antigens are viral antigens as described herein. In some embodiments, one or more viral antigens are from a virus that infects mammals (e.g., humans). In some embodiments, one or more viral antigens are from a latent virus. In some embodiments, one or more viral antigens are viral antigens as described herein.

In some embodiments, one or more viral antigens are viral antigens from a latent virus of the Herpesviridae, Papillomaviridae, Parvoviridae, or Adenoviridae family. In some embodiments, one or more viral antigens are viral antigens from HSV-1, HSV-2, VZV, Human Immunodeficiency Virus (HIV), Epstein-Barr Virus (EBV), CMV, HHV-6, HHV-7, KSHV (HHV-8), JC virus (JCV), BK virus (BKV), parvovirus, or adenovirus.

In some embodiments, one or more viral antigens can comprise T-cell antigens. In some embodiments, one or more viral antigens can comprise B-cell antigens. In some embodiments, one or more viral antigens can comprise T-cell antigens and B-cell antigens.

### RNA Production

Those skilled in the art are aware of a variety of techniques that can be used to produce RNAs as described herein, including chemical or enzymatic (e.g., by polymerization) synthesis. In many embodiments, RNA is produced by transcription, e.g., by in vivo or in vitro transcription. Indeed, one advantage of RNA as an active agent for use in pharmaceutical compositions (e.g., immunogenic compositions, e.g., vaccines) or other therapeutic contexts is its facile production by in vitro transcription. Particularly given that relatively modest adjustments to manufacturing processes can often optimize production of related sequences, the present disclosure teaches that RNA modalities are particularly desirable for use as active agents in pharmaceutical compositions (e.g., immunogenic compositions, e.g., vaccines). Moreover, the present disclosure provides a particular insight that RNA is particularly useful as an active agent in viral vaccines, as it permits facile adaptation (e.g., sequence alteration) to emerging or locally-relevant strains and/or epitopes (e.g., permitting customization of antigen sequences in light of, for example, circulating strains and/or HLA allele diversity within relevant populations (e.g., in a particular geography/region). Furthermore, as the production of RNA requires only a single development and manufacturing platform, irrespective of the encoded pathogen antigens. Thus, RNA has the potential of rapid, cost-efficient, high-volume manufacturing and flexible stockpiling (long term storage of low-volume libraries of frozen plasmid and unformulated RNA, which can be rapidly formulated and distributed). Particularly for viral infection, where timing of administration (e.g., vaccine administration) relative to season and/or incidence of outbreak may materially impact effectiveness, such ability to store and promptly reconstitute may prove and important advantage with critical benefits relative to alternative strategies.

Typically, RNA is transcribed in vitro from a linearized (e.g., by restriction digestion) or amplified (e.g., PCR-amplified) DNA template. Those skilled in the art are aware of a variety of promoters useful for directing RNA synthesis by a transcription of a DNA template, for example by a DNA-dependent RNA polymerase such as, for example, T7, T3, SP6, or Syn5 RNA polymerase.

A typical in vitro transcription reaction will include a DNA template, rNTPs for the four bases (i.e., adenine, cytosine, guanine and uracil), optionally a cap analog, the relevant RNA polymerase, and appropriate buffers and/or salts. In some embodiments, one or more of a ribonuclease (RNase) inhibitor and/or a pyrophosphatase may be included.

In some embodiments, rNTPs utilized in an in vitro transcription reaction include one or more nucleotide analogs. In some embodiments, a nucleotide analog may be selected from the group consisting of 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-0-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-0-(1-thiophosphate)-adenosine, 5′-0-(1-thiophosphate)-cytidine, 5′-0-(1-thiophosphate)-guanosine, 5′-0-(1-thiophosphate)-uridine, 5′-0-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine, pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-0-methyl uridine, and combinations thereof.

In some embodiments, uridine analog(s) are utilized. In some embodiments, no natural uridine is utilized. Thus, in some embodiments 100% of the uracil in the coding sequence have a chemical modification (relative to uridine); in may embodiments at 5-position of the uracil. In particular embodiments, pseudouridine is utilized.

In particular embodiments, utilized nucleotide analogs are selected from the group consisting of pseudouridine, N1-methylpseudouridine, 5-methylcytosine, -methoxyuridine, and combinations thereof.

In some embodiments, the four rNTPs are utilized in equimolar concentrations in in vitro transcription reactions; in some embodiments, they are not equimolar. For example, in some embodiments, one or more rNTPs is at a relatively lower concentration and, in some embodiments, may be supplemented by one or more “feedings” over time during the reaction. In some particular embodiments, rGTP is fed over time (so that the IVT reaction is a “G fed batch” process). Alternatively or additionally, in some particular embodiments, rUTP is fed over time (so that the IVT reaction is a “U fed batch” or “G/U fed batch” process.

In some embodiments, one or more of rNTP concentration, salt concentration, metal concentration, pH, temperature etc, is adjusted for production of a particular RNA construct in order to optimize, for example, one or more of RNA integrity, capping efficiency, contaminant (e.g., dsRNA) level, intact transcript level (e.g., relative to template DNA concentration in the reaction), etc.

In some embodiments, exemplary reagents used in RNA in vitro transcription include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); optionally, a cap analogue as defined herein (e.g. m7G(5′)ppp(5′)G (m7G)); optionally, further modified nucleotides as defined herein; a DNA-dependent RNA polymerase capable of binding to the promoter sequence within the DNA template (e.g. T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCl2, which supplies Mg2+ ions as a co-factor for the polymerase; a buffer (TRIS or HEPES) to maintain a suitable pH value, which can also contain antioxidants (e.g. OTT), and/or polyamines such as spermidine at optimal concentrations, e.g. a buffer system comprising TRIS-Citrate as disclosed in WO2017/109161.

In the context of RNA production, in some embodiments, it may be desired to provide GMP-grade RNA. In some embodiments, GMP-grade RNA may be produced using a manufacturing process approved by regulatory authorities. In some embodiments, RNA production is performed under current good manufacturing practice (GMP), implementing various quality control steps on DNA and/or RNA level, for example, in some embodiments according to quality steps described in WO2016/180430. In some embodiments, RNA of the present disclosure is a GMP-grade RNA.

### DNA Constructs

Among other things, the present disclosure provides DNA constructs, for example that may encode one or more antigens as described herein, or components thereof. In some embodiments, DNA constructs provided by and/or utilized in accordance with the present disclosure are comprised in a vector.

Non-limiting examples of a vector include plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). In some embodiments, a vector is an expression vector. In some embodiments, a vector is a cloning vector. In general, a vector is a nucleic acid construct that can receive or otherwise become linked to a nucleic acid element of interest (e.g., a construct that is or encodes a payload, or that imparts a particular functionality, etc.)

Expression vectors, which may be plasmid or viral or other vectors, typically include an expressible sequence of interest (e.g., a coding sequence) that is functionally linked with one or more control elements (e.g., promoters, enhancers, transcription terminators, etc). Typically, such control elements are selected for expression in a system of interest. In some embodiments, a system is ex vivo (e.g., an in vitro transcription system); in some embodiments, a system is in vivo (e.g., a bacterial, yeast, plant, insect, fish, vertebrate, mammalian cell or tissue, etc).

Cloning vectors are generally used to modify, engineer, and/or duplicate (e.g., by replication in vivo, for example in a simple system such as bacteria or yeast, or in vitro, such as by amplification such as polymerase chain reaction or other amplification process). In some embodiments, a cloning vector may lack expression signals.

In many embodiments, a vector may include replication elements such as primer binding site(s) and/or origin(s) of replication. In many embodiments, a vector may include insertion or modification sites such as restriction endonuclease recognition sites and/or guide RNA binding sites, etc.

In some embodiments, a vector is a viral vector. In some embodiments, a vector is a non-viral vector. In some embodiments, a vector is a plasmid.

Those skilled in the art are aware of a variety of technologies useful for the production of recombinant polynucleotides (e.g., DNA or RNA) as described herein. For example, restriction digestion, reverse transcription, amplification (e.g., by polymerase chain reaction), Gibson assembly, modification by TALENS, CRISPR/Cas enzymes, etc, are well established and useful tools and technologies. Alternatively or additionally, certain nucleic acids may be prepared or assembled by chemical and/or enzymatic synthesis. In some embodiments, a combination of known methods is utilized to prepare a recombinant polynucleotide.

In some embodiments, polynucleotide(s) of the present disclosure are included in a construct (e.g., a vector) amenable to transcription and/or translation.

In some embodiments, a construct of expression amenable to expression comprises a polynucleotide that encodes proteins and/or polypeptides of the present disclosure operatively linked to a sequence or sequences that control expression (e.g., promoters, start signals, stop signals, polyadenylation signals, activators, repressors, etc.). In some embodiments, a sequence or sequences that control expression are selected to achieve a desired level of expression. In some embodiments, more than one sequence that controls expression (e.g., promoters) are utilized. In some embodiments, more than one sequence that controls expression (e.g., promoters) are utilized to achieve a desired level of expression of a plurality of polynucleotides that encode a plurality proteins and/or polypeptides. In some embodiments, a plurality of recombinant proteins and/or polypeptides are expressed from the same vector (e.g., a bi-cistronic vector, a tri-cistronic vector, multi-cistronic). In some embodiments, a plurality of polypeptides are expressed, each of which is expressed from a separate vector.

In some embodiments, a construct amenable to expression comprising a polynucleotide of the present disclosure is used to produce a RNA and/or protein and/or polypeptide in a host cell. In some embodiments, a host cell may be in vitro (e.g., a cell line)—for example a cell or cell line (e.g., Human Embryonic Kidney (HEK cells), Chinese Hamster Ovary cells, etc.) suitable for producing polynucleotides of the present disclosure and proteins and/or polypeptides encoded by said polynucleotides.

A variety of methods are known in the art to introduce a vector into host cells. In some embodiments, a vector may be introduced into host cells using transfection. In some embodiments, transfection is completed, for example, using calcium phosphate transfection, lipofection, or polyethylenimine-mediated transfection. In some embodiments, a vector may be introduced into a host cell using transduction.

In some embodiments, transformed host cells are cultured following introduction of a vector into a host cell to allow for expression of said recombinant polynucleotides. In some embodiments, a transformed host cells are cultured for at least 12 hours, 16 hours, 20 hours, 24 hours, 28 hours, 32 hours, 36 hours 40 hours, 44 hours, 48 hours, 52 hours, 56 hours, 60 hours, 64 hours, 68 hours, 72 hours or longer. Transformed host cells are cultured in growth conditions (e.g., temperature, carbon-dioxide levels, growth medium) in accordance with the requirements of a host cell selected. A skilled artisan would recognize culture conditions for host cells selected are well known in the art.

### Nucleic Acid Containing Particles

Nucleic acids described herein such as RNA encoding a vaccine antigen may be administered formulated as particles.

In the context of the present disclosure, the term “particle” relates to a structured entity formed by molecules or molecule complexes. In one embodiment, the term “particle” relates to a micro- or nano-sized structure, such as a micro- or nano-sized compact structure dispersed in a medium. In one embodiment, a particle is a nucleic acid containing particle such as a particle comprising DNA, RNA or a mixture thereof.

Electrostatic interactions between positively charged molecules such as polymers and lipids and negatively charged nucleic acid are involved in particle formation. This results in complexation and spontaneous formation of nucleic acid particles. In one embodiment, a nucleic acid particle is a nanoparticle.

As used in the present disclosure, “nanoparticle” refers to a particle having an average diameter suitable for parenteral administration.

A “nucleic acid particle” can be used to deliver nucleic acid to a target site of interest (e.g., cell, tissue, organ, and the like). A nucleic acid particle may be formed from at least one cationic or cationically ionizable lipid or lipid-like material, at least one cationic polymer such as protamine, or a mixture thereof and nucleic acid. Nucleic acid particles include lipid nanoparticle (LNP)-based and lipoplex (LPX)-based formulations.

Without intending to be bound by any theory, it is believed that the cationic or cationically ionizable lipid or lipid-like material and/or the cationic polymer combine together with the nucleic acid to form aggregates, and this aggregation results in colloidally stable particles.

In one embodiment, particles described herein further comprise at least one lipid or lipid-like material other than a cationic or cationically ionizable lipid or lipid-like material, at least one polymer other than a cationic polymer, or a mixture thereof.

In some embodiments, nucleic acid particles comprise more than one type of nucleic acid molecules, where the molecular parameters of the nucleic acid molecules may be similar or different from each other, like with respect to molar mass or fundamental structural elements such as molecular architecture, capping, coding regions or other features.

Nucleic acid particles described herein may have an average diameter that in one embodiment ranges from about 30 nm to about 1000 nm, from about 50 nm to about 800 nm, from about 70 nm to about 600 nm, from about 90 nm to about 400 nm, or from about 100 nm to about 300 nm.

Nucleic acid particles described herein may exhibit a polydispersity index less than about 0.5, less than about 0.4, less than about 0.3, or about 0.2 or less. By way of example, the nucleic acid particles can exhibit a polydispersity index in a range of about 0.1 to about 0.3 or about 0.2 to about 0.3.

With respect to RNA lipid particles, the N/P ratio gives the ratio of the nitrogen groups in the lipid to the number of phosphate groups in the RNA. It is correlated to the charge ratio, as the nitrogen atoms (depending on the pH) are usually positively charged and the phosphate groups are negatively charged. The N/P ratio, where a charge equilibrium exists, depends on the pH. Lipid formulations are frequently formed at N/P ratios larger than four up to twelve, because positively charged nanoparticles are considered favorable for transfection. In that case, RNA is considered to be completely bound to nanoparticles.

Nucleic acid particles described herein can be prepared using a wide range of methods that may involve obtaining a colloid from at least one cationic or cationically ionizable lipid or lipid-like material and/or at least one cationic polymer and mixing the colloid with nucleic acid to obtain nucleic acid particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and/or at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion. Using the ethanol injection technique, such colloidal liposome dispersion is, in one embodiment, formed as follows: an ethanol solution comprising lipids, such as cationic lipids and additional lipids, is injected into an aqueous solution under stirring. In one embodiment, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

LNPs typically comprise four components: ionizable cationic lipids, neutral lipids such as phospholipids, a steroid such as cholesterol, and a polymer conjugated lipid such as polyethylene glycol (PEG)-lipids. Each component is responsible for payload protection, and enables effective intracellular delivery. LNPs may be prepared by mixing lipids dissolved in ethanol rapidly with nucleic acid in an aqueous buffer.

The term “average diameter” refers to the mean hydrodynamic diameter of particles as measured by dynamic laser light scattering (DLS) with data analysis using the so-called cumulant algorithm, which provides as results the so-called Z_{average} with the dimension of a length, and the polydispersity index (PI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

The “polydispersity index” is preferably calculated based on dynamic light scattering measurements by the so-called cumulant analysis as mentioned in the definition of the “average diameter”. Under certain prerequisites, it can be taken as a measure of the size distribution of an ensemble of nanoparticles.

Different types of nucleic acid containing particles have been described previously to be suitable for delivery of nucleic acid in particulate form (e.g. Kaczmarek, J. C. et al., 2017, Genome Medicine 9, 60). For non-viral nucleic acid delivery vehicles, nanoparticle encapsulation of nucleic acid physically protects nucleic acid from degradation and, depending on the specific chemistry, can aid in cellular uptake and endosomal escape.

The present disclosure describes particles comprising nucleic acid, at least one cationic or cationically ionizable lipid or lipid-like material, and/or at least one cationic polymer which associate with nucleic acid to form nucleic acid particles and compositions comprising such particles. The nucleic acid particles may comprise nucleic acid which is complexed in different forms by non-covalent interactions to the particle. The particles described herein are not viral particles, in particular infectious viral particles, i.e., they are not able to virally infect cells.

Suitable cationic or cationically ionizable lipids or lipid-like materials and cationic polymers are those that form nucleic acid particles and are included by the term “particle forming components” or “particle forming agents”. The term “particle forming components” or “particle forming agents” relates to any components which associate with nucleic acid to form nucleic acid particles. Such components include any component which can be part of nucleic acid particles.

In some embodiments, a nucleic acid containing particle (e.g., a lipid nanoparticle (LNP)) comprises two or more RNA molecules, each comprising a different nucleic acid sequence. In some embodiments, a nucleic acid containing particle comprises two or more RNA molecules, each encoding a different immunogenic polypeptide or immunogenic fragment thereof. In some embodiments, two or more RNA molecules present in a nucleic acid containing particle comprise: a first RNA molecule encodes an immunogenic polypeptide or immunogenic fragment thereof from a coronavirus and a second RNA molecule encodes an immunogenic polypeptide or immunogenic fragment thereof from an infectious disease pathogen (e.g., virus, bacteria, parasite, etc.).

### Cationic Polymer

Given their high degree of chemical flexibility, polymers are commonly used materials for nanoparticle-based delivery. Typically, cationic polymers are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic polymers herein. In addition, some investigators have synthesized polymers specifically for nucleic acid delivery. Poly(β-amino esters), in particular, have gained widespread use in nucleic acid delivery owing to their ease of synthesis and biodegradability. Such synthetic polymers are also suitable as cationic polymers herein.

A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties such as those described herein.

If more than one type of repeat unit is present within the polymer, then the polymer is said to be a “copolymer.” It is to be understood that the polymer being employed herein can be a copolymer. The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, the polymer is biocompatible. Biocompatible polymers are polymers that typically do not result in significant cell death at moderate concentrations. In certain embodiments, the biocompatible polymer is biodegradable, i.e., the polymer is able to degrade, chemically and/or biologically, within a physiological environment, such as within the body.

In certain embodiments, polymer may be protamine or polyalkyleneimine, in particular protamine.

The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

According to the disclosure, the term “protamine” as used herein is meant to comprise any protamine amino acid sequence obtained or derived from natural or biological sources including fragments thereof and multimeric forms of said amino acid sequence or fragment thereof as well as (synthesized) polypeptides which are artificial and specifically designed for specific purposes and cannot be isolated from native or biological sources.

In one embodiment, the polyalkyleneimine comprises polyethylenimine and/or polypropylenimine, preferably polyethyleneimine. A preferred polyalkyleneimine is polyethyleneimine (PEI). The average molecular weight of PEI is preferably 0.75·10² to 10⁷ Da, preferably 1000 to 10¹ Da, more preferably 10000 to 40000 Da, more preferably 15000 to 30000 Da, even more preferably 20000 to 25000 Da.

Preferred according to the disclosure is linear polyalkyleneimine such as linear polyethyleneimine (PEI).

Cationic polymers (including polycationic polymers) contemplated for use herein include any cationic polymers which are able to electrostatically bind nucleic acid. In one embodiment, cationic polymers contemplated for use herein include any cationic polymers with which nucleic acid can be associated, e.g. by forming complexes with the nucleic acid or forming vesicles in which the nucleic acid is enclosed or encapsulated.

Particles described herein may also comprise polymers other than cationic polymers, i.e., non-cationic polymers and/or anionic polymers. Collectively, anionic and neutral polymers are referred to herein as non-cationic polymers.

### Lipid and Lipid-Like Material

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids.

In certain embodiments, the amphiphilic compound is a lipid. The term “lipid” refers to a group of organic compounds that are characterized by being insoluble in water, but soluble in many organic solvents. Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

### Cationic or Cationically Ionizable Lipids or Lipid-Like Materials

The nucleic acid particles described herein may comprise at least one cationic or cationically ionizable lipid or lipid-like material as particle forming agent. Cationic or cationically ionizable lipids or lipid-like materials contemplated for use herein include any cationic or cationically ionizable lipids or lipid-like materials which are able to electrostatically bind nucleic acid. In one embodiment, cationic or cationically ionizable lipids or lipid-like materials contemplated for use herein can be associated with nucleic acid, e.g. by forming complexes with the nucleic acid or forming vesicles in which the nucleic acid is enclosed or encapsulated.

As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

For purposes of the present disclosure, such “cationically ionizable” lipids or lipid-like materials are comprised by the term “cationic lipid or lipid-like material” unless contradicted by the circumstances.

In one embodiment, the cationic or cationically ionizable lipid or lipid-like material comprises a head group which includes at least one nitrogen atom (N) which is positive charged or capable of being protonated.

Examples of cationic lipids include, but are not limited to 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spenmine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (+)—N-(3-aminopropyl)-N,N-dimethyl-2,3-bis(cis-9-tetradecenyloxy)-1-propanaminium bromide (GAP-DMORIE), (+)—N-(3-aminopropyl)-N,N-dimethyl-2,3-bis(dodecyloxy)-1-propanaminium bromide (GAP-DLRIE), (+)—N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (PAE-DMRIE), N-(4-carboxybenzyl)-N,N-dimethyl-2,3-bis(oleoyloxy)propan-1-aminium (DOBAQ), 2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-ammonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N₁₂-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In some embodiments, the cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle.

### Additional Lipids or Lipid-Like Materials

Particles described herein may also comprise lipids or lipid-like materials other than cationic or cationically ionizable lipids or lipid-like materials, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. Optimizing the formulation of nucleic acid particles by addition of other hydrophobic moieties, such as cholesterol and lipids, in addition to an ionizable/cationic lipid or lipid-like material may enhance particle stability and efficacy of nucleic acid delivery.

An additional lipid or lipid-like material may be incorporated which may or may not affect the overall charge of the nucleic acid particles. In certain embodiments, the additional lipid or lipid-like material is a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH. In preferred embodiments, the additional lipid comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain preferred embodiments, the additional lipid is DSPC or DSPC and cholesterol.

In certain embodiments, the nucleic acid particles include both a cationic lipid and an additional lipid.

In one embodiment, particles described herein include a polymer conjugated lipid such as a pegylated lipid. The term “pegylated lipid” refers to a molecule comprising both a lipid portion and a polyethylene glycol portion. Pegylated lipids are known in the art.

Without wishing to be bound by theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some embodiments, the molar ratio of the at least one cationic lipid to the at least one additional lipid is from about 10:0 to about 1:9, about 4:1 to about 1:2, or about 3:1 to about 1:1.

In some embodiments, the non-cationic lipid, in particular neutral lipid, (e.g., one or more phospholipids and/or cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in the particle.

### Lipoplex Particles

In certain embodiments of the present disclosure, the RNA described herein may be present in RNA lipoplex particles.

In the context of the present disclosure, the term “RNA lipoplex particle” relates to a particle that contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, a RNA lipoplex particle is a nanoparticle.

In certain embodiments, the RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

RNA lipoplex particles described herein have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

The RNA lipoplex particles and compositions comprising RNA lipoplex particles described herein are useful for delivery of RNA to a target tissue after parenteral administration, in particular after intravenous administration. The RNA lipoplex particles may be prepared using liposomes that may be obtained by injecting a solution of the lipids in ethanol into water or a suitable aqueous phase. In one embodiment, the aqueous phase has an acidic pH. In one embodiment, the aqueous phase comprises acetic acid, e.g., in an amount of about 5 mM. Liposomes may be used for preparing RNA lipoplex particles by mixing the liposomes with RNA. In one embodiment, the liposomes and RNA lipoplex particles comprise at least one cationic lipid and at least one additional lipid. In one embodiment, the at least one cationic lipid comprises 1,2-di-O-octadecenyl-3-trimethylammonium propane (DOTMA) and/or 1,2-dioleoyl-3-trimethylammonium-propane (DOTAP). In one embodiment, the at least one additional lipid comprises 1,2-di-(9Z-octadecenoyl)-sn-glycero-3-phosphoethanolamine (DOPE), cholesterol (Chol) and/or 1,2-dioleoyl-sn-glycero-3-phosphocholine (DOPC). In one embodiment, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In one embodiment, the liposomes and RNA lipoplex particles comprise 1,2-di-O-octadecenyl-3-trimethylammonium propane (DOTMA) and 1,2-di-(9Z-octadecenoyl)-sn-glycero-3-phosphoethanolamine (DOPE).

Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

### Lipid Nanoparticles (LNPs)

In one embodiment, nucleic acid such as RNA described herein is administered in the form of lipid nanoparticles (LNPs). The LNP may comprise any lipid capable of forming a particle to which the one or more nucleic acid molecules are attached, or in which the one or more nucleic acid molecules are encapsulated.

In one embodiment, the LNP comprises one or more cationic lipids, and one or more stabilizing lipids. Stabilizing lipids include neutral lipids and pegylated lipids.

In one embodiment, the LNP comprises a cationic lipid, a neutral lipid, a steroid, a polymer conjugated lipid; and the RNA, encapsulated within or associated with the lipid nanoparticle.

In one embodiment, the LNP comprises from 40 to 55 mol percent, from 40 to 50 mol percent, from 41 to 49 mol percent, from 41 to 48 mol percent, from 42 to 48 mol percent, from 43 to 48 mol percent, from 44 to 48 mol percent, from 45 to 48 mol percent, from 46 to 48 mol percent, from 47 to 48 mol percent, or from 47.2 to 47.8 mol percent of the cationic lipid. In one embodiment, the LNP comprises about 47.0, 47.1, 47.2, 47.3, 47.4, 47.5, 47.6, 47.7, 47.8, 47.9 or 48.0 mol percent of the cationic lipid.

In one embodiment, the neutral lipid is present in a concentration ranging from 5 to 15 mol percent, from 7 to 13 mol percent, or from 9 to 11 mol percent. In one embodiment, the neutral lipid is present in a concentration of about 9.5, 10 or 10.5 mol percent.

In one embodiment, the steroid is present in a concentration ranging from 30 to 50 mol percent, from 35 to 45 mol percent or from 38 to 43 mol percent. In one embodiment, the steroid is present in a concentration of about 40, 41, 42, 43, 44, 45 or 46 mol percent.

In one embodiment, the LNP comprises from 1 to 10 mol percent, from 1 to 5 mol percent, or from 1 to 2.5 mol percent of the polymer conjugated lipid.

In one embodiment, the LNP comprises from 40 to 50 mol percent a cationic lipid; from 5 to 15 mol percent of a neutral lipid; from 35 to 45 mol percent of a steroid; from 1 to 10 mol percent of a polymer conjugated lipid; and the RNA, encapsulated within or associated with the lipid nanoparticle.

In one embodiment, the mol percent is determined based on total mol of lipid present in the lipid nanoparticle.

In one embodiment, the neutral lipid is selected from the group consisting of DSPC, DPPC, DMPC, DOPC, POPC, DOPE, DOPG, DPPG, POPE, DPPE, DMPE, DSPE, and SM. In one embodiment, the neutral lipid is selected from the group consisting of DSPC, DPPC, DMPC, DOPC, POPC, DOPE and SM. In one embodiment, the neutral lipid is DSPC.

In one embodiment, the steroid is cholesterol.

In one embodiment, the polymer conjugated lipid is a pegylated lipid. In one embodiment, the pegylated lipid has the following structure:

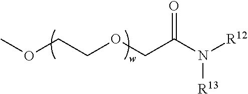

or a pharmaceutically acceptable salt, tautomer or stereoisomer thereof, wherein R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In one embodiment, R¹² and R¹³ are each independently straight, saturated alkyl chains containing from 12 to 16 carbon atoms. In one embodiment, w has a mean value ranging from 40 to 55. In one embodiment, the average w is about 45. In one embodiment, R¹² and R¹³ are each independently a straight, saturated alkyl chain containing about 14 carbon atoms, and w has a mean value of about 45.

In one embodiment, the pegylated lipid is DMG-PEG 2000, e.g., having the following structure:

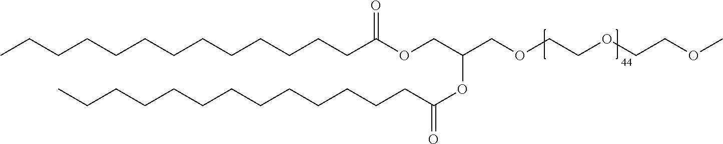

In some embodiments, the cationic lipid component of the LNPs has the structure of Formula (III):

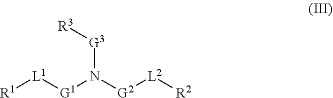

or a pharmaceutically acceptable salt, tautomer, prodrug or stereoisomer thereof, wherein one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (11111):

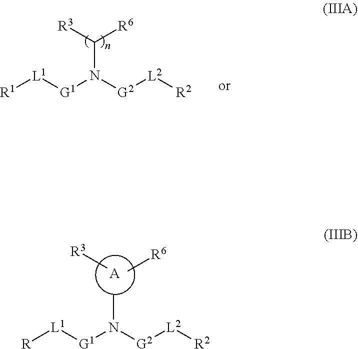

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

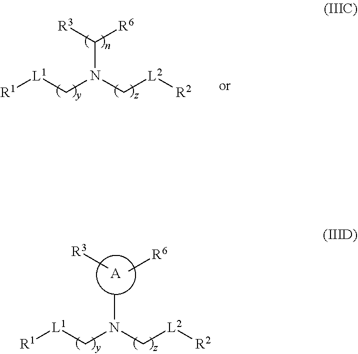

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)—.

In some different embodiments of Formula (III), the lipid has one of the following structures (IIIE) or (IIIF):

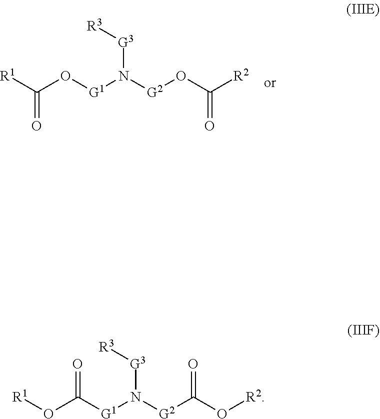

In some of the foregoing embodiments of Formula (III), the lipid has one of the following structures (IIIG), (IIIH), (IIII), or (IIIJ):

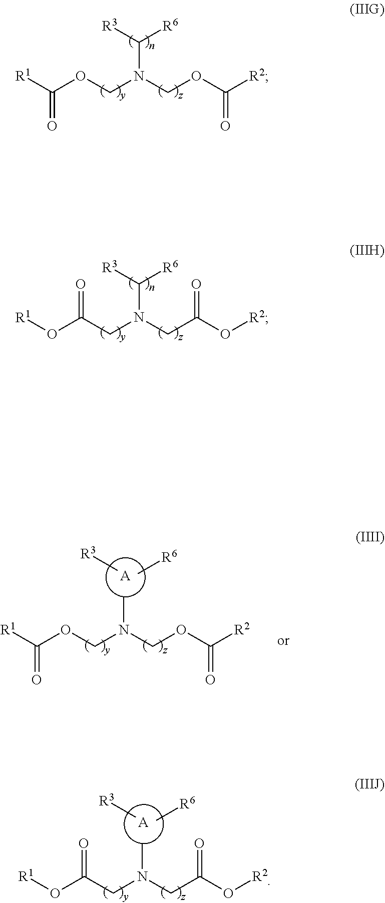

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G³ is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

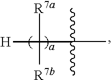

wherein:
R^{7a} and R^{7b} are, at each occurrence, independently H or C₁-C₁₂ alkyl; and
a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

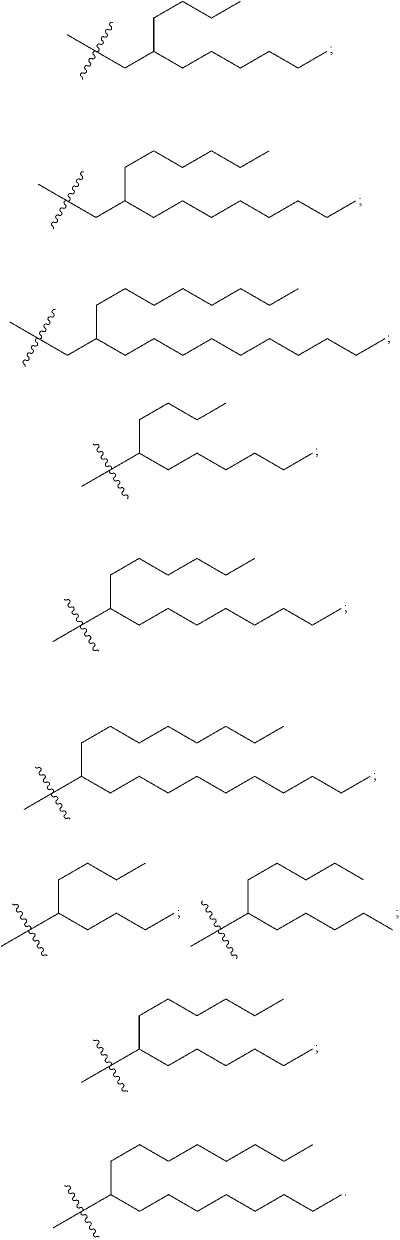

In some of the foregoing embodiments of Formula (III), R is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in the table below.

TABLE 5
Representative Compounds of Formula (III).

| No. | Structure |
|---|---|
| III-1 | 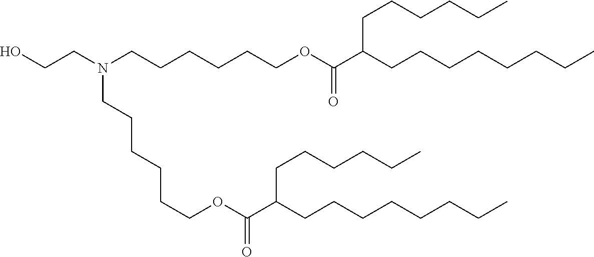 [SMILES: CCCCCCCCC(CCCCCC)C(=O)OCCCCCCN(CCO)CCCCCCOC(=O)C(CCCCCC)CCCCCCCC] |
| III-2 | 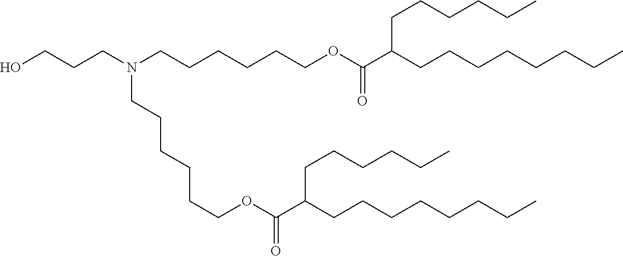 [SMILES: CCCCCCCCC(CCCCCC)C(=O)OCCCCCCN(CCCO)CCCCCCOC(=O)C(CCCCCC)CCCCCCCC] |
| III-3 | 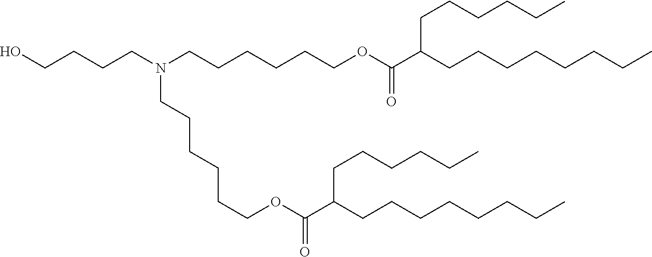 [SMILES: CCCCCCCCC(CCCCCC)C(=O)OCCCCCCN(CCCCO)CCCCCCOC(=O)C(CCCCCC)CCCCCCCC] |
| III-4 | 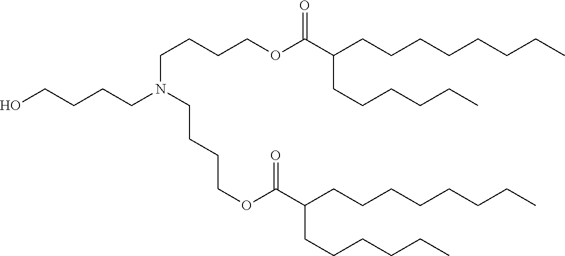 [SMILES: CCCCCCCCC(CCCCCC)C(=O)OCCCCN(CCCCO)CCCCOC(=O)C(CCCCCC)CCCCCCCC] |
| III-5 | 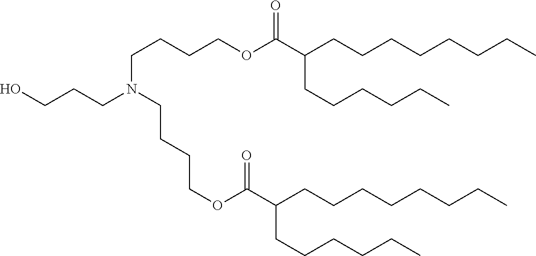 [SMILES: CCCCCCCCC(CCCCCC)C(=O)OCCCCN(CCCO)CCCCOC(=O)C(CCCCCC)CCCCCCCC] |
| III-6 | 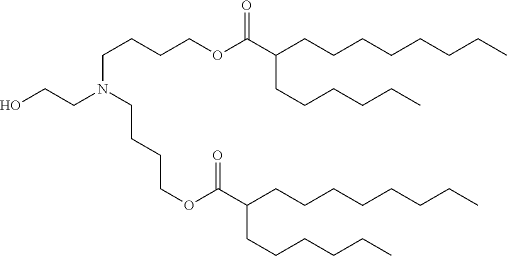 [SMILES: CCCCCCCCC(CCCCCC)C(=O)OCCCCN(CCO)CCCCOC(=O)C(CCCCCC)CCCCCCCC] |
| III-7 | 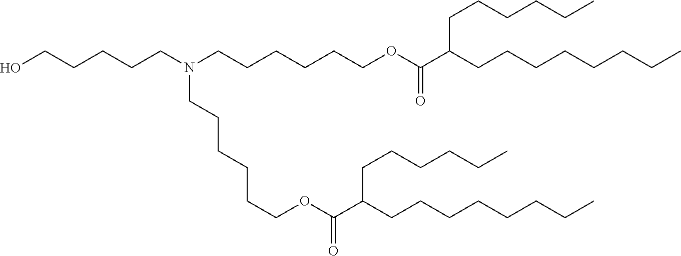 [SMILES: CCCCCCCCC(CCCCCC)C(=O)OCCCCCCN(CCCCCO)CCCCCCOC(=O)C(CCCCCC)CCCCCCCC] |
| III-8 | 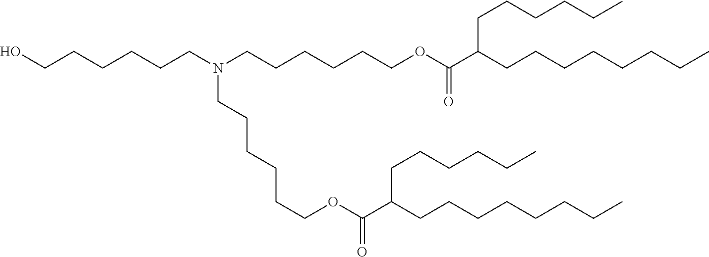 [SMILES: CCCCCCCCC(CCCCCC)C(=O)OCCCCCCN(CCCCCCO)CCCCCCOC(=O)C(CCCCCC)CCCCCCCC] |
| III-9 | 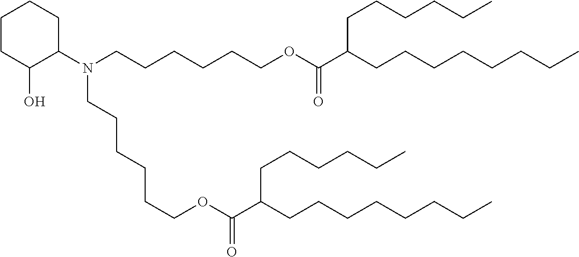 [SMILES: CCCCCCCCC(CCCCCC)C(=O)OCCCCCCN(CCCCCCOC(=O)C(CCCCCC)CCCCCCCC)C1CCCCC1O] |
| III-10 | 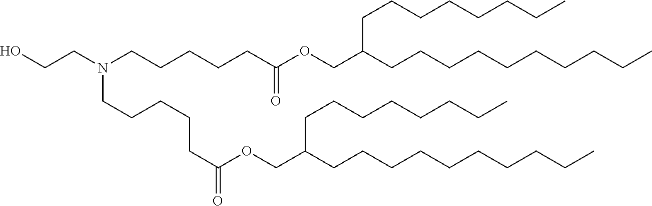 [SMILES: CCCCCCCCCCC(CCCCCCCC)COC(=O)CCCCCN(CCO)CCCCCC(=O)OCC(CCCCCCCC)CCCCCCCCCC] |
| III-11 | 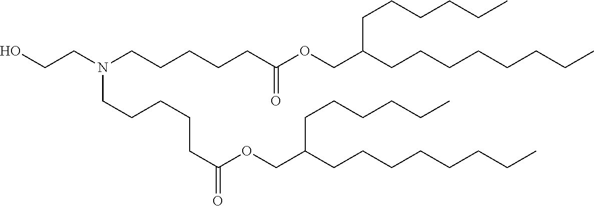 [SMILES: CCCCCCCCC(CCCCCC)COC(=O)CCCCCN(CCO)CCCCCC(=O)OCC(CCCCCC)CCCCCCCC] |
| III-12 | 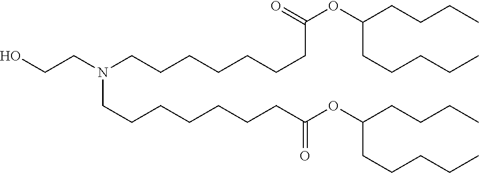 [SMILES: CCCCCC(CCCC)OC(=O)CCCCCCCN(CCO)CCCCCCCC(=O)OC(CCCC)CCCCC] |
| III-13 | 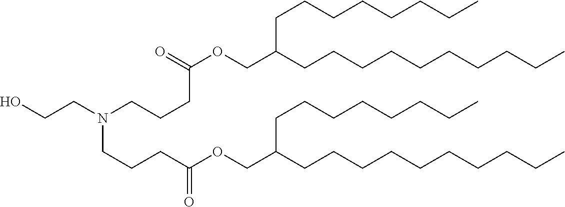 [SMILES: CCCCCCCCCCC(CCCCCCCC)COC(=O)CCCN(CCO)CCCC(=O)OCC(CCCCCCCC)CCCCCCCCCC] |
| III-14 | 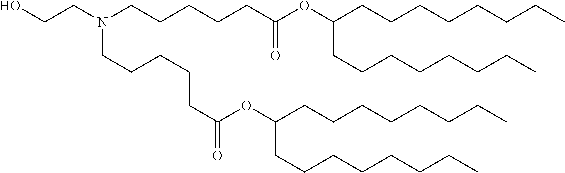 [SMILES: CCCCCCCCC(CCCCCCCC)OC(=O)CCCCCN(CCO)CCCCCC(=O)OC(CCCCCCCC)CCCCCCCC] |
| III-15 | 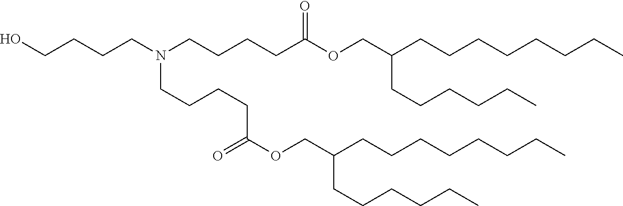 [SMILES: CCCCCCCCC(CCCCCC)COC(=O)CCCCN(CCCCO)CCCCC(=O)OCC(CCCCCC)CCCCCCCC] |
| III-16 | 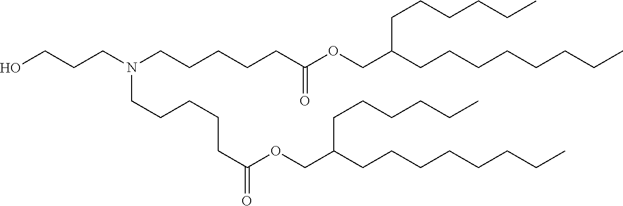 [SMILES: CCCCCCCCC(CCCCCC)COC(=O)CCCCCN(CCCO)CCCCCC(=O)OCC(CCCCCC)CCCCCCCC] |
| III-17 | 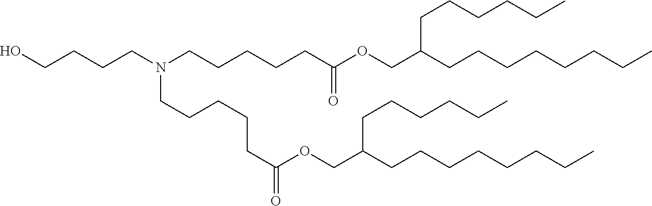 [SMILES: CCCCCCCCC(CCCCCC)COC(=O)CCCCCN(CCCCO)CCCCCC(=O)OCC(CCCCCC)CCCCCCCC] |
| III-18 | 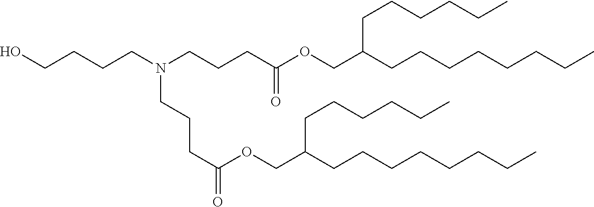 [SMILES: CCCCCCCCC(CCCCCC)COC(=O)CCCN(CCCCO)CCCC(=O)OCC(CCCCCC)CCCCCCCC] |
| III-19 | 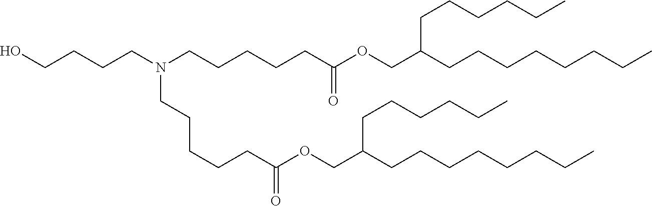 [SMILES: CCCCCCCCC(CCCCCC)COC(=O)CCCCCN(CCCCO)CCCCCC(=O)OCC(CCCCCC)CCCCCCCC] |
| III-20 | 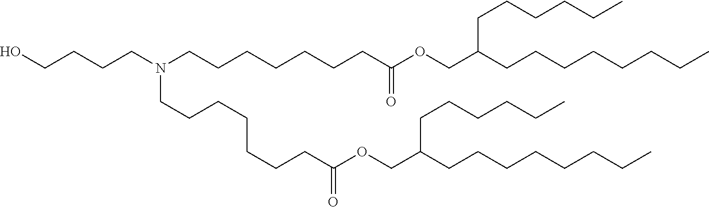 [SMILES: CCCCCCCCC(CCCCCC)COC(=O)CCCCCCCN(CCCCO)CCCCCCCC(=O)OCC(CCCCCC)CCCCCCCC] |
| III-21 | 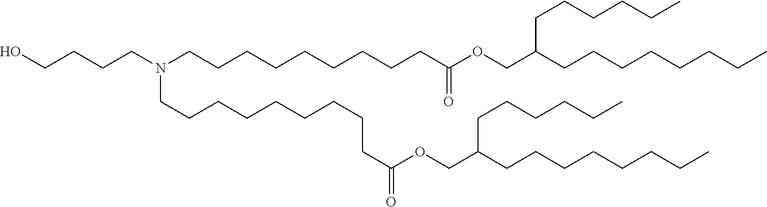 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 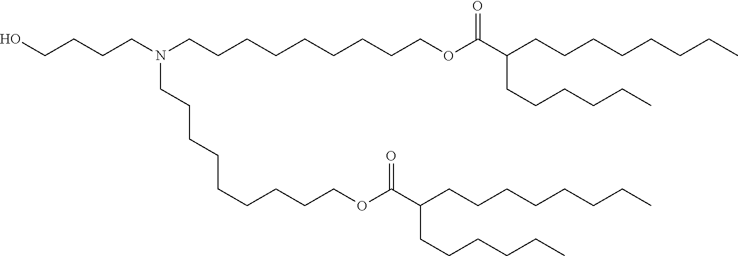 [SMILES: CCCCCCCCC(CCCCCC)C(=O)OCCCCCCCCCN(CCCCO)CCCCCCCCCOC(=O)C(CCCCCC)CCCCCCCC] |
| III-23 | 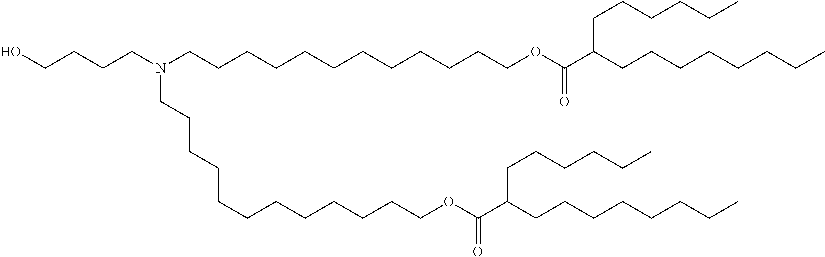 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 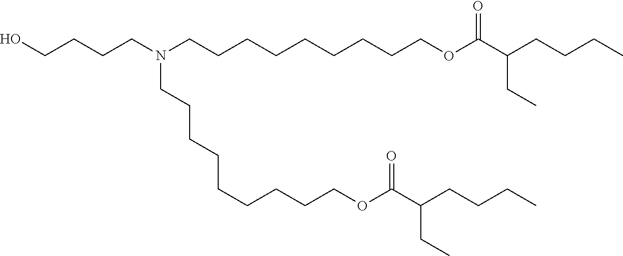 [SMILES: CCCCC(CC)C(=O)OCCCCCCCCCN(CCCCO)CCCCCCCCCOC(=O)C(CC)CCCC] |
| III-25 | 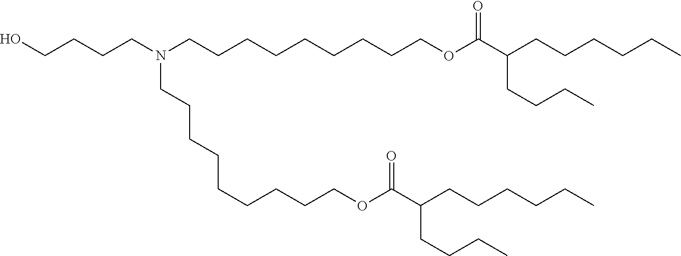 [SMILES: CCCCCCC(CCCC)C(=O)OCCCCCCCCCN(CCCCO)CCCCCCCCCOC(=O)C(CCCC)CCCCCC] |
| III-26 | 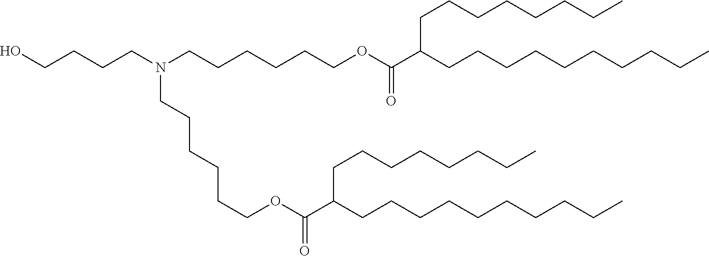 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 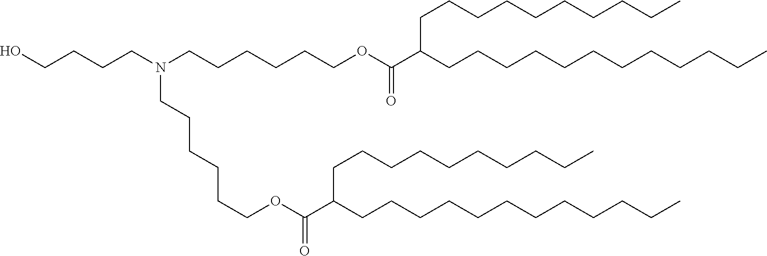 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 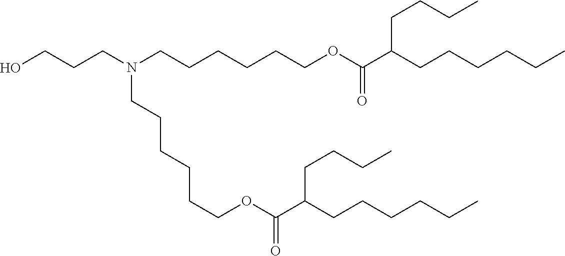 [SMILES: CCCCCCC(CCCC)C(=O)OCCCCCCN(CCCO)CCCCCCOC(=O)C(CCCC)CCCCCC] |
| III-29 | 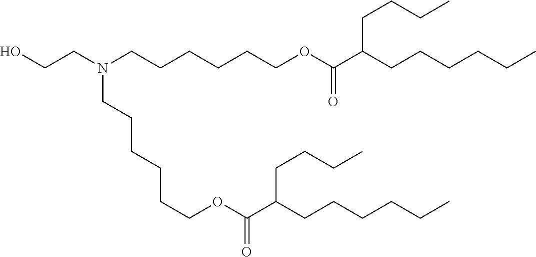 [SMILES: CCCCCCC(CCCC)C(=O)OCCCCCCN(CCO)CCCCCCOC(=O)C(CCCC)CCCCCC] |
| III-30 | 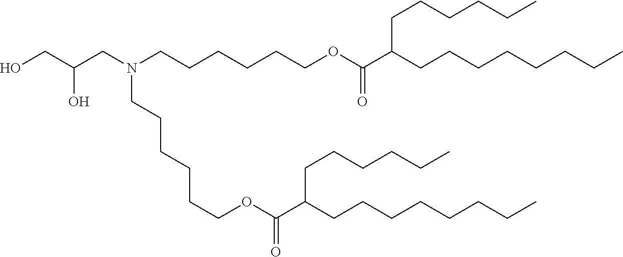 [SMILES: CCCCCCCCC(CCCCCC)C(=O)OCCCCCCN(CCCCCCOC(=O)C(CCCCCC)CCCCCCCC)CC(O)CO] |
| III-31 | 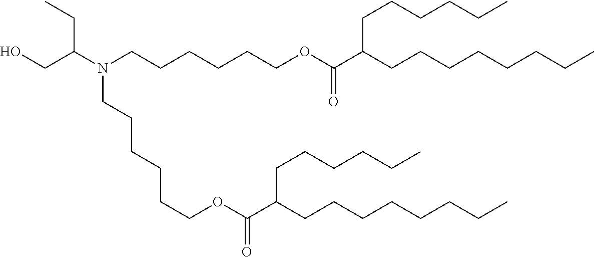 [SMILES: CCCCCCCCC(CCCCCC)C(=O)OCCCCCCN(CCCCCCOC(=O)C(CCCCCC)CCCCCCCC)C(CC)CO] |
| III-32 | 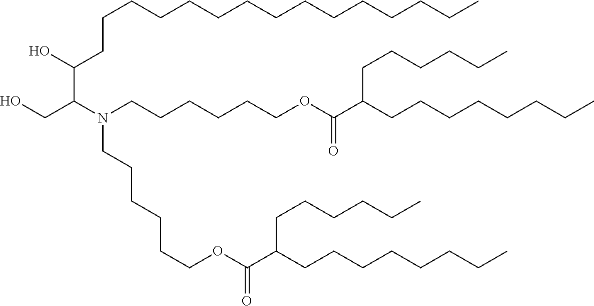 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 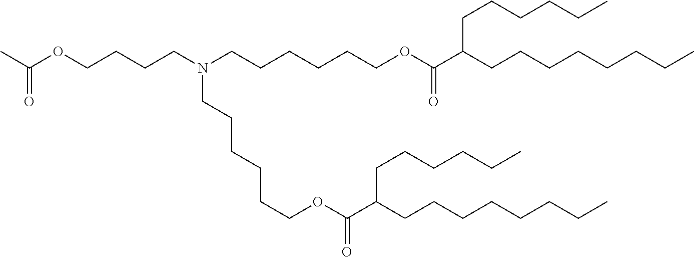 [SMILES: CCCCCCCCC(CCCCCC)C(=O)OCCCCCCN(CCCCCCOC(=O)C(CCCCCC)CCCCCCCC)CCCCOC(C)=O] |
| III-34 | 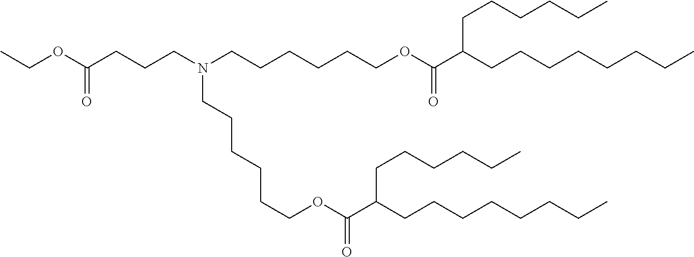 [SMILES: CCCCCCCCC(CCCCCC)C(=O)OCCCCCCN(CCCCCCOC(=O)C(CCCCCC)CCCCCCCC)CCCC(=O)OCC] |
| III-35 | 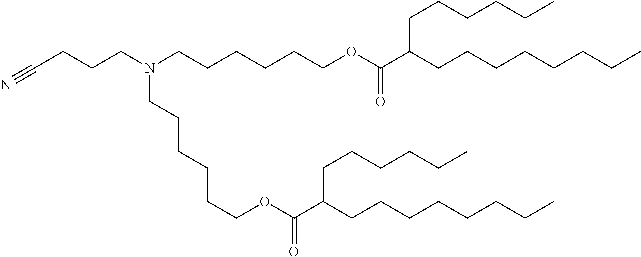 [SMILES: CCCCCCCCC(CCCCCC)C(=O)OCCCCCCN(CCCC#N)CCCCCCOC(=O)C(CCCCCC)CCCCCCCC] |
| III-36 | 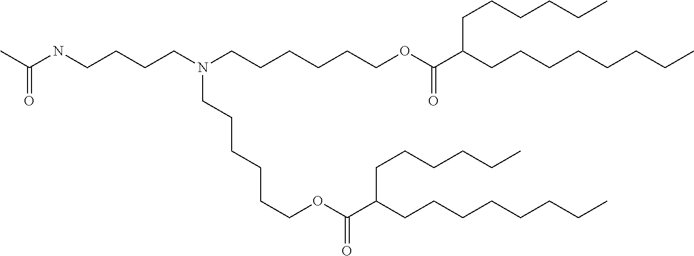 [SMILES: CCCCCCCCC(CCCCCC)C(=O)OCCCCCCN(CCCCCCOC(=O)C(CCCCCC)CCCCCCCC)CCCCNC(C)=O] |

In some embodiments, the LNP comprises a lipid of Formula (III), RNA, a neutral lipid, a steroid and a pegylated lipid. In some embodiments, the lipid of Formula (III) is compound III-3. In some embodiments, the neutral lipid is DSPC. In some embodiments, the steroid is cholesterol. In some embodiments, the pegylated lipid is ALC-0159.

In some embodiments, the cationic lipid is present in the LNP in an amount from about 40 to about 50 mole percent. In one embodiment, the neutral lipid is present in the LNP in an amount from about 5 to about 15 mole percent. In one embodiment, the steroid is present in the LNP in an amount from about 35 to about 45 mole percent. In one embodiment, the pegylated lipid is present in the LNP in an amount from about 1 to about 10 mole percent.

In some embodiments, the LNP comprises compound III-3 in an amount from about 40 to about 50 mole percent, DSPC in an amount from about 5 to about 15 mole percent, cholesterol in an amount from about 35 to about 45 mole percent, and ALC-0159 in an amount from about 1 to about 10 mole percent.

In some embodiments, the LNP comprises compound III-3 in an amount of about 47.5 mole percent, DSPC in an amount of about 10 mole percent, cholesterol in an amount of about 40.7 mole percent, and ALC-0159 in an amount of about 1.8 mole percent.

In various different embodiments, the cationic lipid has one of the structures set forth in the table below.

TABLE 19
Representative cationic lipids.

| No. | Structure |
|---|---|
| A | 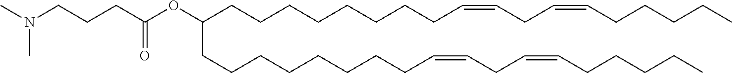 [SMILES: CCCCC/C=C\C/C=C\CCCCCCCCC(CCCCCCCC/C=C\C/C=C\CCCCC)OC(=O)CCCN(C)C] |
| B | 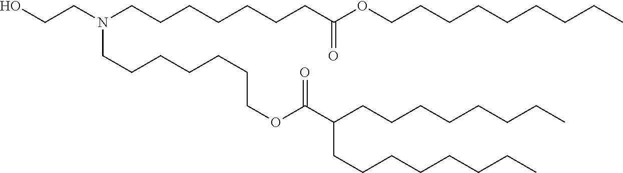 [SMILES: CCCCCCCCCOC(=O)CCCCCCCN(CCO)CCCCCCCOC(=O)C(CCCCCCCC)CCCCCCCC] |
| C | 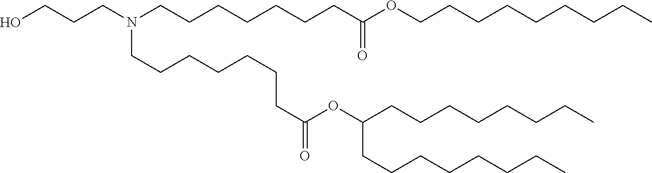 [SMILES: CCCCCCCCCOC(=O)CCCCCCCN(CCCO)CCCCCCCC(=O)OC(CCCCCCCC)CCCCCCCC] |
| D | 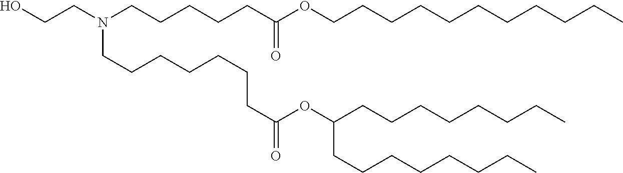 [SMILES: CCCCCCCCCCCOC(=O)CCCCCN(CCO)CCCCCCCC(=O)OC(CCCCCCCC)CCCCCCCC] |
| E | 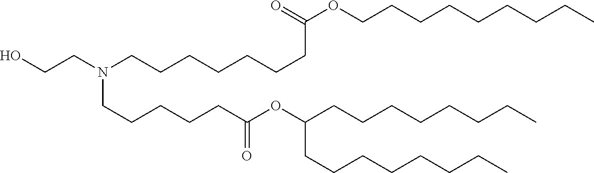 [SMILES: CCCCCCCCCOC(=O)CCCCCCCN(CCO)CCCCCC(=O)OC(CCCCCCCC)CCCCCCCC] |
| F | 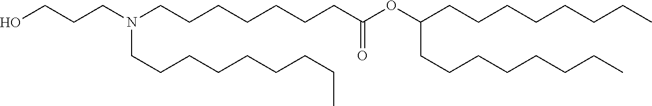 [SMILES: CCCCCCCCCN(CCCO)CCCCCCCC(=O)OC(CCCCCCCC)CCCCCCCC] |

In some embodiments, the LNP comprises a cationic lipid shown in the above table, e.g., a cationic lipid of Formula (B) or Formula (D), in particular a cationic lipid of Formula (D), RNA, a neutral lipid, a steroid and a pegylated lipid. In some embodiments, the neutral lipid is DSPC. In some embodiments, the steroid is cholesterol. In some embodiments, the pegylated lipid is DMG-PEG 2000.

In one embodiment, the LNP comprises a cationic lipid that is an ionizable lipid-like material (lipidoid). In one embodiment, the cationic lipid has the following structure:

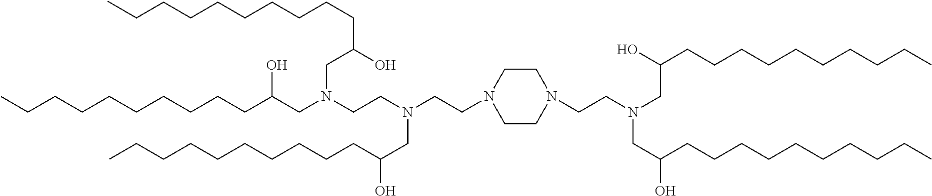 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

The N/P value is preferably at least about 4. In some embodiments, the N/P value ranges from 4 to 20, 4 to 12, 4 to 10, 4 to 8, or 5 to 7. In one embodiment, the N/P value is about 6.

LNP described herein may have an average diameter that in one embodiment ranges from about 30 nm to about 200 nm, or from about 60 nm to about 120 nm.

### Formulations and Pharmaceutical Compositions

The present disclosure provides a variety of embodiments of pharmaceutical compositions (e.g., immunogenic compositions, e.g., vaccines) appropriate for administration to humans that, for example, deliver viral antigens as described herein.

Typically, provided formulations comprise an active agent and one or more excipients or carriers.

In some embodiments, an active agent may be or comprise a viral antigen—e.g., that is or comprises a viral protein or fragment or epitope thereof, as described herein. Thus, in some embodiments, an active agent is a polypeptide or plurality of polypeptides. In some embodiments, a polypeptide active agent includes a plurality of viral antigen epitopes (e.g., from a single viral protein or from a plurality of different viral proteins). In some embodiments, a polypeptide active agent is or comprises at least one peptide that represents a distinct viral epitope. In some embodiments, a polypeptide active agent includes at least one peptide that is or comprises a fragment or epitope of a viral protein; in some such embodiments, the polypeptide active agent does not include any full-length viral protein.

In some embodiments, an active agent may be or comprise a cell population—for example a population of cells that expresses (e.g., internally, on its surface, and or secreting) at least one antigen as described herein. Alternatively or additionally, a population of cells (e.g., antigen presenting cells such as dendritic cells) that are loaded with (e.g., bound in MHC complexes) viral antigen peptides as described herein.

In some embodiments, an active agent is a polynucleotide that encodes (or is complementary to one that encodes) a viral antigen as described herein. In some such embodiments, a polynucleotide is single-stranded; in other embodiments, a polynucleotide is double stranded. In some embodiments, a polynucleotide active agent is DNA (e.g., a DNA viral vector, such as an adenoviral, adeno-associated viral, baculoviral, poxviral [e.g., vaccinia viral] vector); in some embodiments, a polynucleotide active agent is RNA (e.g., a lentiviral vector or, more preferably, an mRNA construct as described herein).

In many embodiments, a polynucleotide active agent is RNA and is provided and/or utilized in a lipid composition such as a lipoplex preparation or, preferably, an LNP preparation.

In some embodiments, a provided formulation is a liquid formulation. In some embodiments, a provided formulation is a solid (e.g., frozen formulation. In some embodiments, a provided formulation is a dry formulation.

Those skilled in the art will appreciate that nature and/or content of formulations may be different for different active agents (e.g., polypeptide vs nucleic acid [e.g., as LNP] vs cell). Regardless, in most embodiments, compositions will be formulated (or amenable to formulation) for parenteral (e.g., subQ, IM, or IV) administration. LNP embodiments are typically formulated, or amenable to formulation, by injection.

In many embodiments, formulations (particularly frozen or dry formulations, or liquid formulations to be frozen or dried, or prepared from frozen or dry preparations), may include one or more stabilizing agents such as, for example, trehalose, sucrose, mannose, dextran and/or inulin.

In some embodiments, a nucleic acid (e.g., DNA or RNA) composition, particularly if it is not an LNP preparation, may include a complexing agent or transfection agent. In some embodiments, a nucleic acid may be complexed with protamine.

In some embodiments, an RNA composition is formulated in an aminoalcohol lipidoid.

In some embodiments, a liquid composition is in an aqueous carrier such as, for example, water, typically pyrogen-free water; isotonic saline or buffered (aqueous) solutions, e.g. phosphate, citrate etc. buffered solutions. In some embodiments, Ringer-Lactate solution is used. In some embodiments, a buffered solution such as is described in WO2006/122828 is utilized.

In some embodiments, a buffer system or other solvent includes a sodium salt, preferably at least 50 mM of a sodium salt, a calcium salt, preferably at least 0.01 mM of a calcium salt, and optionally a potassium salt, preferably at least 3 mM of a potassium salt. In some embodiments, sodium, calcium and, optionally, potassium salts may occur in the form of their halogenides, e.g. chlorides, iodides, or bromides, in the form of their hydroxides, carbonates, hydrogen carbonates, or sulfates, etc. Examples of sodium salts include NaCl, NaI, NaBr, Na₂CO₃, NaHCO₃, Na₂SO₄, examples of the optional potassium salts include KCl, Kl, KBr, K₂CO₃, KHCO₃, K2₂SO₄, and examples of calcium salts include CaCl₂), CaI₂, CaBr₂, CaCO₃, CaSQ₄, Ca(OH)₂. Alternatively or additional, in some embodiments, one or more organic ions of such salts may be present.

In some embodiments, a provided composition is formulated, for example, with one or more emulsifiers, such as for example, Tween; wetting agents, such as, for example, sodium lauryl sulfate; colouring agents; taste-imparting agents, pharmaceutical carriers; tablet-forming agents; stabilizers; antioxidants; preservatives.

In some embodiments, provided compositions are sterile and/or pyrogen free.

In some embodiments, provided compositions are in a stock format that, for example, requires dilution prior to use. In some embodiments, provided compositions are in a ready-to-use format that does not require dilution prior to use. In either option, in some embodiments, thawing and/or resuspension is required for use.

In some embodiments, provided compositions are in single dose format; in other embodiments, provided compositions are in multidose format.

In some embodiments, provided compositions are disposed in a container such as, for example, a bag (e.g., a shipping bag or an IV bag, a vial, a syringe, etc).

In some embodiments, provided compositions are amenable to storage, or have been stored, for a period of time and/or under particular conditions (e.g., temperature, light exposure, pH, pressure, etc)

In some embodiments, a provided formulation does not include an adjuvant (other than the active itself). In some embodiments, a provided formulation does not include 3-O-desacyl-4′-monophosphoryl lipid A (MPL), and/or a saponin, QS-21.

### Characterization

Without wishing to be bound by any particular theory, it is proposed that ability to induce CD8⁺ T cells may be important to effectiveness of a composition for the treatment of viral infection (e.g., a pharmaceutical composition, an immunogenic composition, or a vaccine). Alternatively or additionally, in some embodiments, a robust antibody response may be required for effectiveness. In some embodiments, it may be that both are required or useful.

In some embodiments, provided technologies (e.g., compositions and/or dosing regimens, etc) are characterized by an ability to induce (e.g., when administered to a model system and/or to a human, for example by parenteral administration such as by intramuscular administration) an immune response characterized by CD8⁺ T cells targeting one or more viral antigen(s) described herein. That is, in some embodiments, provided technologies are characterized in that, when administered (e.g., by parenteral administration such as by intramuscular administration) to an organism (e.g., a model organism or an animal or human organism in need of protection), provided technologies induce CD8+ T cells targeting one or more viral antigens. In some embodiments, provided technologies are characterized in that they induce a greater CD8⁺ T cell response with respect to one or more viral antigens and/or induce a CD8⁺ T cell response with broader diversity (e.g., with detectable and/or significant binding to a larger number of different T cell epitopes) than is observed for a known vaccine for the same virus or another appropriate reference.

In some embodiments, provided technologies are characterized in that they induce gammadelta T cells. As those skilled in the art are aware, gammadelta T cells typically represent only a small fraction (e.g., up to about 5%) of an overall T cell population in an organism. Gammadelta T cells express TCR chains encoded by the gamma and delta gene loci; subsets of gamma delta T cells are defined by the inclusion of invariant TCR V-(D)-J segments and are tissue- or context-specific. Gammadelta T cells secrete particular effector cytokines in a subtype- and context-specific manner. Often, gammadelta T cells express certain markers (e.g., as Fc gamma RIII/CD16 and Toll-like receptors that are often associated with natural killer cells and/or antigen-presenting cells. Gammadelta T cells typically lack CD4 and CD8.

In some embodiments, provided technologies are characterized in that they induce antibodies of type and level sufficient to block virus development, assembly, replication, immune evasion, and/or host-cell invasion. In some embodiments, provided technologies are characterized in that they induce polyclonal high affinity antibodies.

In some embodiments, provided technologies are characterized in that they induce antibody titers to a level that provides sufficient protective response against viral, when administered to a relevant population.

In some embodiments, provided technologies are characterized in that they induce sterile protection, e.g., when evaluated in a model system such as a mouse modes.

In some embodiments, provided technologies are characterized in that they induce a CD4+ T helper cell response and/or CD8+ T cell memory responses (e.g., promoting development and/or expansion of memory CD8+ T cells.

In some embodiments, provided compositions are assessed as described herein, for example for RNA integrity, stability, level, capping efficiency, translatability, of RNA, etc and/or for one or more properties of a composition (e.g., an LNP preparation) such as, for example ability to induce an antibody response, a T cell response, a T cell response with particular features (e.g., level of antibody to one or more epitopes, persistence of such level, diversity of elicited antibodies, type and/or diversity of T cell response, etc.

In some embodiments, provided formulations are identified and/or characterized with respect to one or more activities or features, including, for example, expression level, nature of immune response, level of protection (e.g., to challenge, impact on viral load, impact on health and/or survival), immunogenicity (e.g., assessment of cytokine responses, phenotyping of immune response, T cell depletion and/or protection), serology (e.g., assessment of IgG specific titers, inhibition of life cycle stage development, neutralization, passive transfer), and/or functional antibody responses (e.g., neutralization, inhibition of life cycle stage development, passive transfer). Those skilled in the art will be aware that animal models of human viral infection are limited. Regardless, in many embodiments, assessment of provided compositions in human system(s) is desirable. In some embodiments, in vitro assessments are performed in human systems. To give but a few examples, in some embodiments, presentation of provided antigen(s) or fragments or epitopes thereof by human dendritic cells to stimulate human T cells is assessed in vitro. Alternatively or additionally, in some embodiments, in vitro binding of sera from infected humans to provided antigens is assessed.

In some embodiments, in vivo assessments are performed in human systems. Alternatively or additionally, in some embodiments, subjects are monitored for responsiveness (e.g., increased responsiveness) to a particular known or potential anti-viral infection therapy.

In some embodiments, a provided composition (e.g., an immunostimulatory composition, e.g., a vaccine composition) provides significant (e.g., at least about 60%, 65%, 70%, 75%, 80%, 85%, 90% or more protection from one or more of established infection, symptomatic disease, and/or serious disease. In some embodiments, a provided composition (e.g., an immunostimulatory composition, e.g., a vaccine composition) provides significantly increased responsiveness to therapy, for example as may be assessed by one or more of delayed onset, reduction of severity, and/or faster resolution of one or more symptoms or characteristics of infection.

### Use

In some aspects, technologies of the present disclosure are used for therapeutic and/or prophylactic purposes. In some embodiments, technologies of the present disclosure are used in the treatment and/or prophylactic of an infection with a virus. Prophylactic purposes of the present disclosure comprises pre-exposure prophylaxis and/or post-exposure prophylaxis.

In some embodiments, technologies of the present disclosure are used in the treatment and/or prophylaxis of a disorder related to such an infection. A disordered related to such an infection comprises, for example, a typical symptom and/or a complication of a viral infection.

In some embodiments, provided compositions (e.g., that are or comprise viral antigens) may be useful to detect and/or characterize one or more features of an anti-viral infection immune response (e.g., by detecting binding to a provided antigen by serum from an infected subject).

In some embodiments, provided compositions (e.g., that are or comprise viral antigens) are useful to raise antibodies to one or more epitopes included therein; such antibodies may themselves be useful, for example for detection or treatment of viruses or infection thereby.

The present disclosure provides use of encoding nucleic acids (e.g., DNA or RNA) to produce encoded antigens and/or use of DNA constructs to produce RNA.

### Subject Populations

In some embodiments, technologies of the present disclosure are utilized in a non-limited subject population; in some embodiments, technologies of the present disclosure are utilized in particular subject populations.

In some embodiments, a subject population comprises a pediatric population. In some embodiments, a pediatric population comprises subjects approximately 18 years old or younger. In some such embodiments, a pediatric population comprises subjects between the ages of about 1 year and about 18 years (e.g., 1, 2, 3, 4, 5, 6, 7, 8, 9, 10, 11, 12, 13, 14, 15, 16, 17, or 18 years of age).

In some embodiments, a subject population comprises a newborn population. In some embodiments, a newborn population comprises subjects about 12 months or younger (e.g., 12, 11, 10, 9, 8, 7, 6, 5, 4, 3, 2, 1 months or younger). In some embodiments, subject populations to be treated with technologies described herein include infants (e.g., about 12 months or younger) whose mothers did not receive such technologies described herein during pregnancy. In some embodiments, subject populations to be treated with technologies described herein may include pregnant women; in some embodiments, infants whose mothers were treated with disclosed technologies during pregnancy (e.g., who received at least one dose, or alternatively only who received both doses), are not vaccinated during the first weeks, months, or even years (e.g., 1, 2, 3, 4, 5, 6, 7, 8 weeks or more, or 1, 2, 3, 4, 5, 6, 7, 8, 9, 10, 11, 12, 13, 14, 15, 16, 17, 18, 19, 20, 21, 22, 23, 24 months or more, or 1, 2, 3, 4, 5 years or more) post-birth. Alternatively or additionally, in some embodiments, infants whose mothers were treated with disclosed technologies during pregnancy (e.g., who received at least one dose, or alternatively only who received both doses), receive reduced treated with disclosed technologies (e.g., lower doses and/or smaller numbers of administrations—e.g., boosters—and/or lower total exposure over a given period of time) after birth, for example during the first weeks, months, or even years (e.g., 1, 2, 3, 4, 5, 6, 7, 8 weeks or more, or 1, 2, 3, 4, 5, 6, 7, 8, 9, 10, 11, 12, 13, 14, 15, 16, 17, 18, 19, 20, 21, 22, 23, 24 months or more, or 1, 2, 3, 4, 5 years or more) post-birth or may need reduced vaccination (e.g., lower doses and/or smaller numbers of administrations—e.g., boosters—over a given period of time), In some embodiments, compositions as provided herein are administered to subject populations that do not include pregnant women.

In some embodiments, a subject population is or comprises children aged 6 weeks to up to 17 months of age.

In some embodiments, a subject population comprises an adult population. In some embodiments, an adult population comprises subjects between the ages of about 19 years and about 60 years of age (e.g., about 20, 25, 30, 35, 40, 45, 50, 55, or 60 years of age).

In some embodiments, a subject population comprises an elderly population. In some embodiments, an elderly population comprises subjects of about 60 years of age, about 70 years of age, or older (e.g., about 65, 70, 75, 80, 85, 90, 95, or 100 years of age).

In some embodiments, a subject population comprises a population with a high risk of infection (e.g., viral infection). In some such embodiments, a population may be deemed to have a high risk of infection due to a local epidemic or a global pandemic. In some such embodiments, a population may be deemed to have a high risk of infection due to a subject population's geographic area. In some embodiments, a subject population comprises subjects that have been exposed to infection (e.g., viral infection).

In some embodiments, where a subject population is or includes pregnant women, provided technologies offer a particular advantage of interrupting virus' transmission cycle, including, for example, in some embodiments, by reducing or eliminating transmission from pregnant mothers to their fetuses.

In some embodiments, a subject population is or comprises immunocompromised individuals. In some embodiments, a subject population does not include immunocompromised individuals.

In some embodiments, a provided pharmaceutical composition (e.g., immunogenic composition, e.g., vaccine) may be administered in combination with (i.e., so that subject(s) are simultaneously exposed to both) another pharmaceutical composition (e.g., immunogenic composition, e.g., vaccine) or therapeutic intervention, e.g., to treat or prevent viral infection or another disease, disorder, or condition.

In some embodiments, a provided pharmaceutical composition (e.g., immunogenic composition, e.g., vaccine) may be administered with a protein vaccine, a DNA vaccine, an RNA vaccine, a cellular vaccine, a conjugate vaccine, etc. In some embodiments, one or more doses of a provided pharmaceutical composition (e.g., immunogenic composition, e.g., vaccine) may be administered together with (e.g., in a single visit) another vaccine or other therapy.

In some embodiments, a provided pharmaceutical composition (e.g., immunogenic composition, e.g., vaccine) may be administered to subjects who have been exposed, or expect they have been exposed, to virus. In some embodiments, a provided pharmaceutical composition (e.g., immunogenic composition, e.g., vaccine) may be administered to subjects who do not have symptoms of viral infection.

### Dosing Regimens

In some embodiments, technologies of the present disclosure may be administered to subjects according to a particular dosing regimen. In some embodiments, a dosing regimen may involve a single administration; in some embodiments, a dosing regimen may comprise one or more “booster” administrations after the initial administration. In some embodiments, initial and boost doses are the same amount; in some embodiments they differ. In some embodiments, two or more booster doses are administered. In some embodiments, a plurality of doses are administered at regular intervals. In some embodiments, periods of time between doses become longer. In some embodiments, one or more subsequent doses is administered if a particular clinical (e.g., reduction in neutralizing antibody levels) or situational (e.g., local development of a new strain) even arises or is detected.

In some embodiments, an administered pharmaceutical compositions (e.g., immunogenic compositions, e.g., vaccines) comprising RNA constructs that encode viral antigen(s) are administered in RNA doses of from about 0.1 μg to about 300 μg, about 0.5 μg to about 200 μg, or about 1 μg to about 100 μg, such as about 1 μg, about 3 μg, about 10 μg, about 30 μg, about 50 μg, or about 100 μg. In some embodiments, an saRNA construct is administered at a lower dose (e.g., 2, 4, 5, 10 fold or more lower) than a modRNA or uRNA construct.

In some embodiments, a first booster dose is administered within a about six months of the initial dose, and preferably within about 5, 4, 3, 2, or 1 months. In some embodiments, a first booster dose is administered in a time period that begins about 1, 2, 3, or 4 weeks after the first dose, and ends about 2, 3, 4, 5, 6, 7, 8, 9, 10, 11, 12 weeks of the first dose (e.g., between about 1 and about 12 weeks after the first dose, or between about 2 or 3 weeks and about 5 and 6 weeks after the first dose, or about 3 weeks or about 4 weeks after the first dose).

In some embodiments, a plurality of booster doses (e.g., 2, 3, or 4) doses are administered within 6 months of the first dose, or within 12 months of the first dose.

In some embodiments, 3 doses or fewer are required to achieve effective vaccination (e.g., greater than 60%, and in some embodiments greater than about 70%, about 75%, about 80%, about 85%, about 90% or more) reduction in risk of infection, or of serious disease. In some embodiments, not more than two doses are required. In some embodiments, a single dose is sufficient. In some embodiments, an RNA dose is about 60 μg or lower, 50 μg or lower, 40 μg or lower, 30 μg or lower, 20 μg or lower, 10 μg or lower, 5 μg or lower, 2.5 μg or lower, or 1 μg or lower. In some embodiments, an RNA dose is about 0.25 μg, at least 0.5 μg, at least 1 μg, at least 2 μg, at least 3 μg, at least 4 μg, at least 5 μg, at least 10 μg, at least 20 μg, at least 30 μg, or at least 40 μg. In some embodiments, an RNA dose is about 0.25 μg to 60 μg, 0.5 μg to 55 μg, 1 μg to 50 μg, 5 μg to 40 μg, or 10 μg to 30 μg may be administered per dose. In some embodiments, an RNA dose is about 30 μg. In some embodiments, at least two such doses are administered. For example, a second dose may be administered about 21 days following administration of the first dose. In some embodiments, a viral antigen delivered by a booster dose is a variant (e.g., representing a more current circulating strain) of one administered in a prior dose. In some embodiments, one or more doses delivers a plurality of distinct viral antigens (e.g., one comprising a B cell epitope, and optionally comprising a CD4 epitope, and one comprising a CD8 epitope; alternatively one comprising a first viral protein, or fragment or epitope thereof, and at least one comprising a second viral protein, or fragment or epitope thereof). In some embodiments, where a later dose delivers a plurality of antigens, at least one of the plurality is an antigen administered in a prior dose. In some embodiments, a first booster dose is administered about one month after an initial dose. In some such embodiments, at least one further booster is administered at one-month interval(s). In some embodiments, after 2 or 3 boosters, a longer interval is introduced and no further booster is administered for at least 6, 9, 12, 18, 24, or more months. In some embodiments, a single further booster is administered after about 18 months. In some embodiments, no further booster is required unless, for example, a material change in clinical or environmental situation is observed.

### Exemplification

The present Examples below provide exemplary technologies for designing, making and/or using immunogenic compositions described herein, for example, in some embodiments, immunogenic compositions that can be particularly useful treating and/or preventing latent virus infection. Specifically, the present Examples below provide approaches for identifying regions and/or epitopes of viral polypeptide antigens and design of polynucleotides (e.g., RNA) encoding same, which may be useful for inclusion in immunogenic compositions described herein. The present Examples below also provide exemplary immunogenic compositions comprising or encoding one or more identified regions and/or epitopes or viral polypeptide antigens. In some embodiments, such regions and/or epitopes of viral polypeptide antigens can be expressed by a virus during a latent phase of its life cycle and/or during an active infection. In some embodiments, such regions and/or epitopes of viral polypeptides antigens can be or comprise T cell epitopes. In some embodiments, such regions and/or epitopes of viral polypeptide antigens can be or comprise B cell epitopes. In some embodiments, such regions and/or epitopes of viral polypeptide antigens can be or comprise predominantly T cell epitopes.

For illustrative purposes only, technologies described herein are exemplified for immunogenic compositions that target Varicella Zoster Virus (e.g., Examples 1-14), Cytomegalovirus (e.g., Examples 15-28), and Norovirus (e.g., Examples 29-43). A skilled artisan reading the present disclosure will recognize that similar technologies can be applied to design, make, and/or use immunogenic compositions for various strains of Varicella Zoster Virus, Cytomegalovirus, and Norovirus and/or for treatment and/or prevention of a different viral infection, for example, in some embodiments infections associated with reactivation of latent viruses. Examples of latent viruses that are known to cause typical latent infections in human include, but are not limited to herpesvirus (e.g., HSV-1, HSV-2, varicella-zoster virus, cytomegalovirus, Epstein-Barr virus, etc.), noroviruses, HIV, human polyomavirus 2 (JC virus).

### Example 1: Exemplary Datasets for Varicella Zoster Virus (VZV) and Methods for the Immune Epitope Database (IEDB) Data Curation

The present Example provides exemplary datasets curated for identification of regions and/or epitopes of VZV antigens that may be useful for immunogenic compositions (e.g., vaccine compositions, and epitope binding agents, etc.). In some embodiments, such regions and/or epitopes of VZV antigens can be expressed by VZV during a latent phase of its life cycle and/or during an active infection. In some embodiments, such regions and/or epitopes of VZV antigens can be or comprise T cell epitopes. In some embodiments, such regions and/or epitopes of VZV antigens can be or comprise B cell epitopes. In some embodiments, such regions and/or epitopes of VZV antigens can be or comprise predominantly T cell epitopes.

### Exemplary Datasets Curated for Varicella Zoster Virus (VZV)

HSV1 to VZV gene homology was obtained from Table 1 of Cohen “The varicella-zoster virus genome.” Curr Top Microbiol Immunol. 2010; 342:1-14. doi:10.1007/82_2010_10, the entire contents of which are incorporated herein by reference for the purposes described herein.

RNA-seq expression in TPM for four conditions (i.e., VZV-infected cells treated with cycloheximide (CHX) or phosphonoacetic acid (PAA), or untreated VZV-infected cells at 24 hr post-infection or at 96 hr post-infection) was obtained from Supplementary Table 3 of Braspenning et al. “Decoding the Architecture of the Varicella-Zoster Virus Transcriptome.” mBio. 2020; 11(5):e01568-20. Published 2020 Oct. 6. doi:10.1128/mBio.01568-20, the entire contents of which are incorporated herein by reference for the purposes described herein.

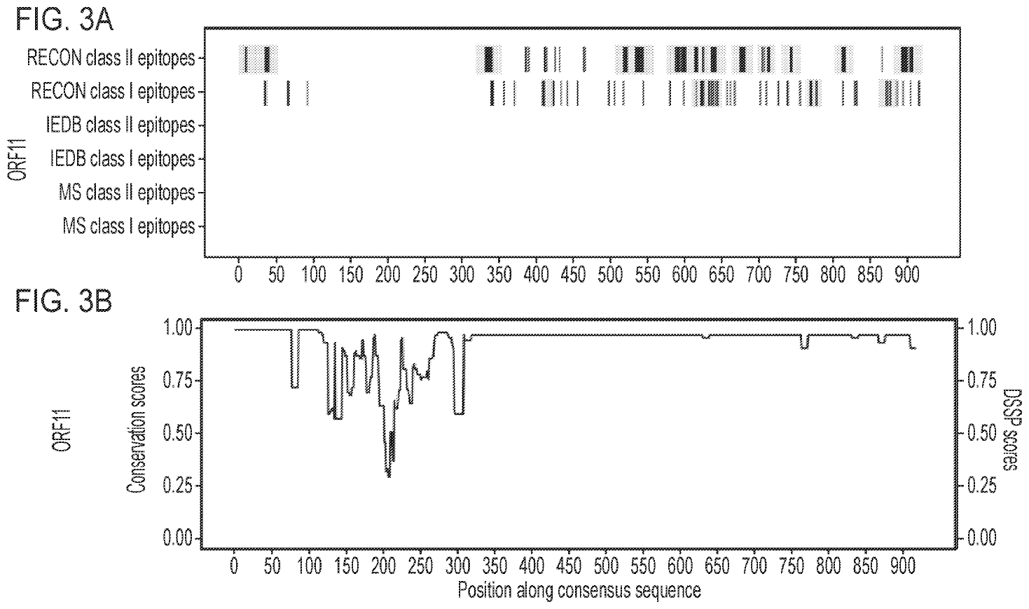

% of the subjects with CD4 response for each VZV protein after vaccination with weakened VZV strain on days 0, 28 and 182 was quantified from FIG. 3A of Laing et al. “Zoster Vaccination Increases the Breadth of CD4+ T Cells Responsive to Varicella Zoster Virus.” J Infect Dis. 2015; 212(7):1022-1031. doi:10.1093/infdis/jiv164, the entire contents of which are incorporated herein by reference for the purposes described herein.

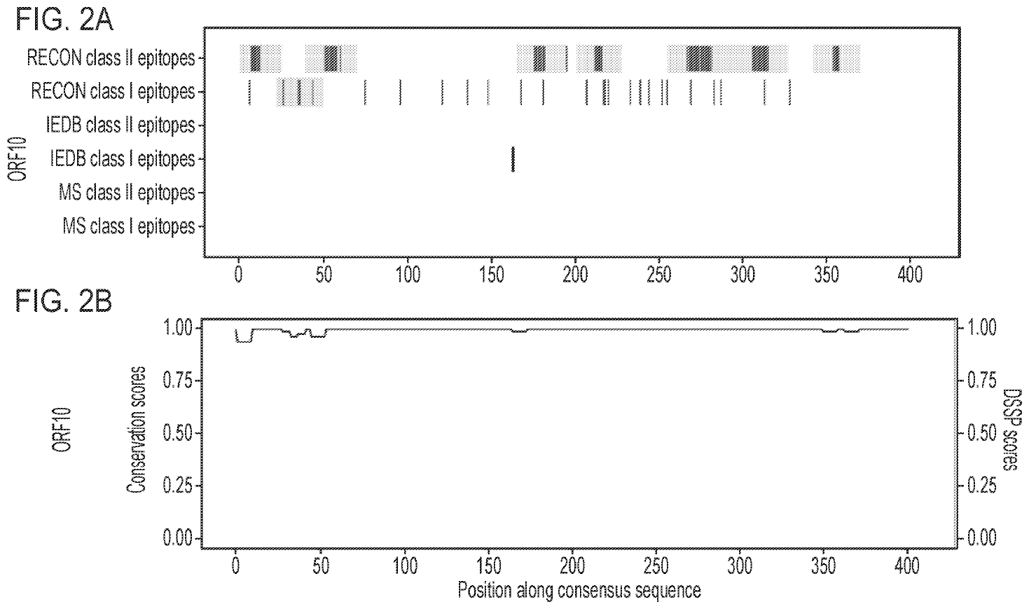

% of the subjects with CD4 response for each VZV protein after vaccination with weakened VZV strain was quantified from FIG. 2A of Jing et al. “Extensive CD4 and CD8 T Cell Cross-Reactivity between Alphaherpesviruses.” J Immunol. 2016; 196(5):2205-2218. doi:10.4049/jinmunol.1502366, the entire contents of which are incorporated herein by reference for the purposes described herein.

Pools of all strongly predicted peptides (top 0.5% using IEDB consensus tool) across all ORFs were evaluated for immunogenicity in 21 subjects with primary VZV infection, detectable VZV IgG, and no previous VZV vaccination. One pool which induced positive responses in 15/21 subjects was deconvolved and tested again using ELISPOT. Two candidate peptides from this pool which induced positive responses were then evaluated via multimer staining. See Chiu et al. “Broadly reactive human CD8 T cells that recognize an epitope conserved between VZV, HSV and EBV,” PLoS Pathog. 2014; 10(3):e1004008. Published 2014 Mar. 27. doi:10.1371/journal.ppat.1004008, the entire contents of which are incorporated herein by reference for the purposes described herein.

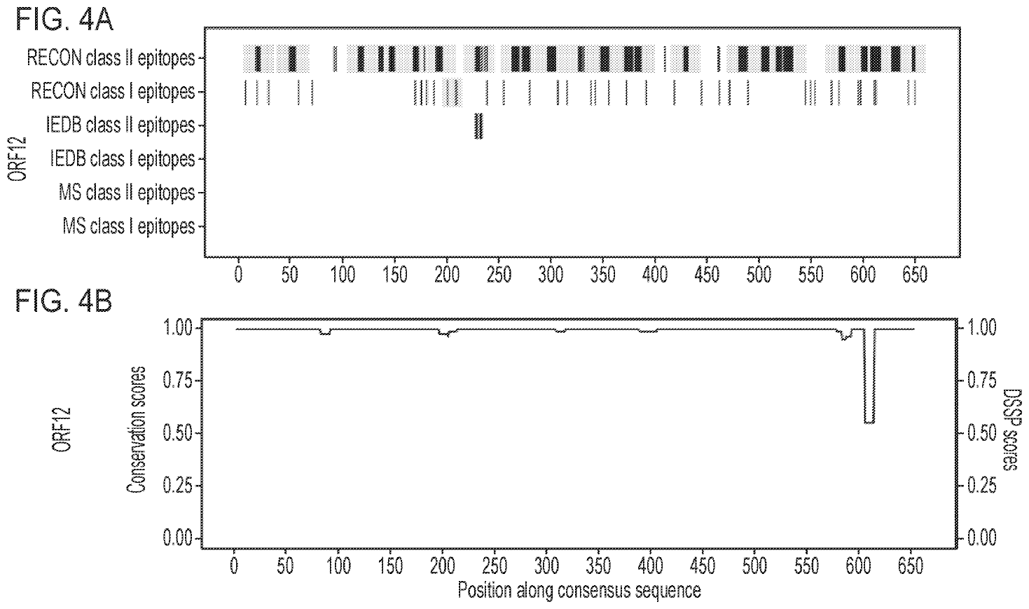

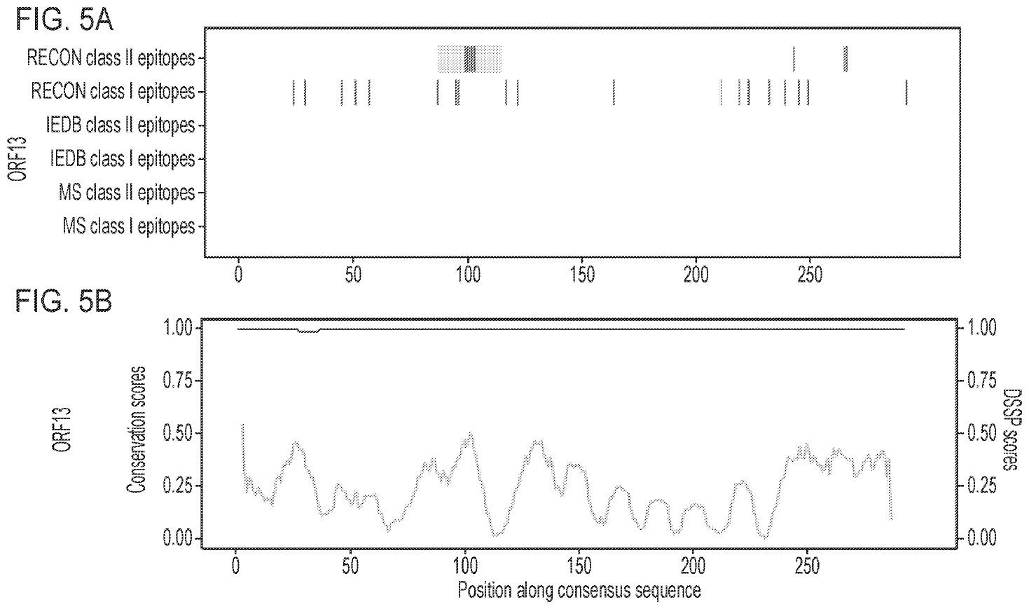

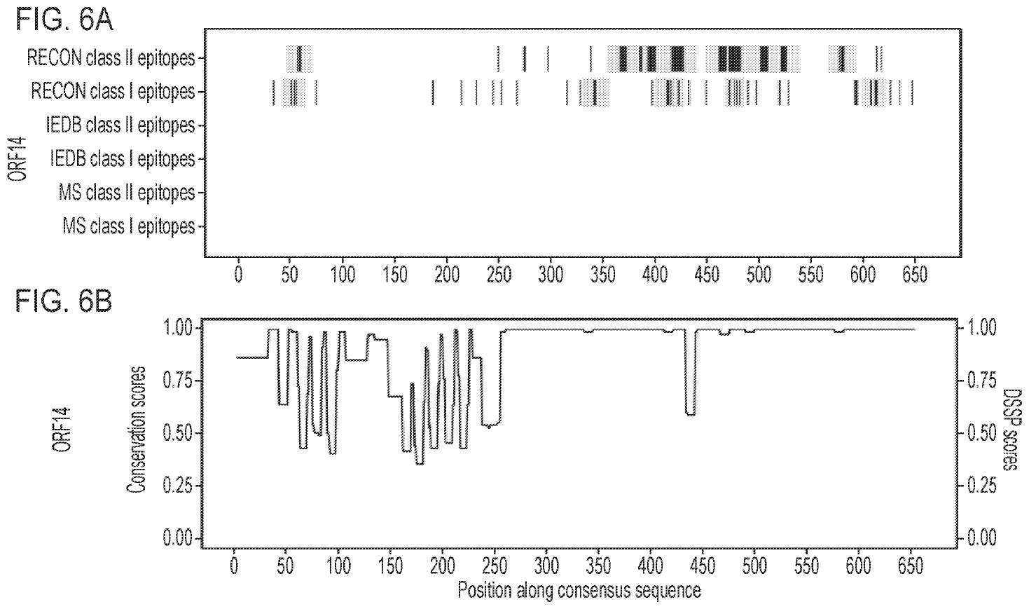

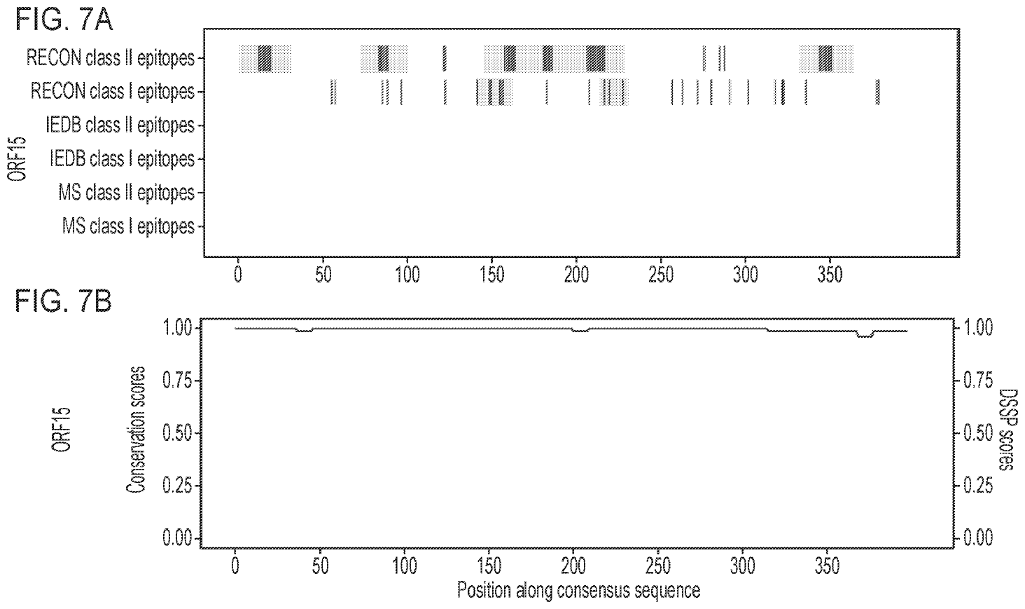

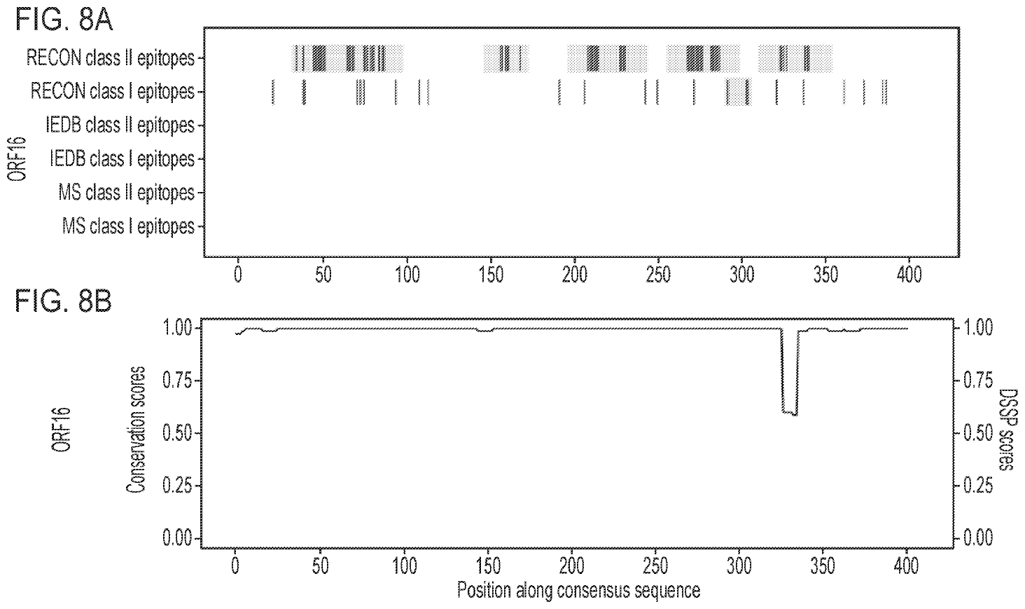

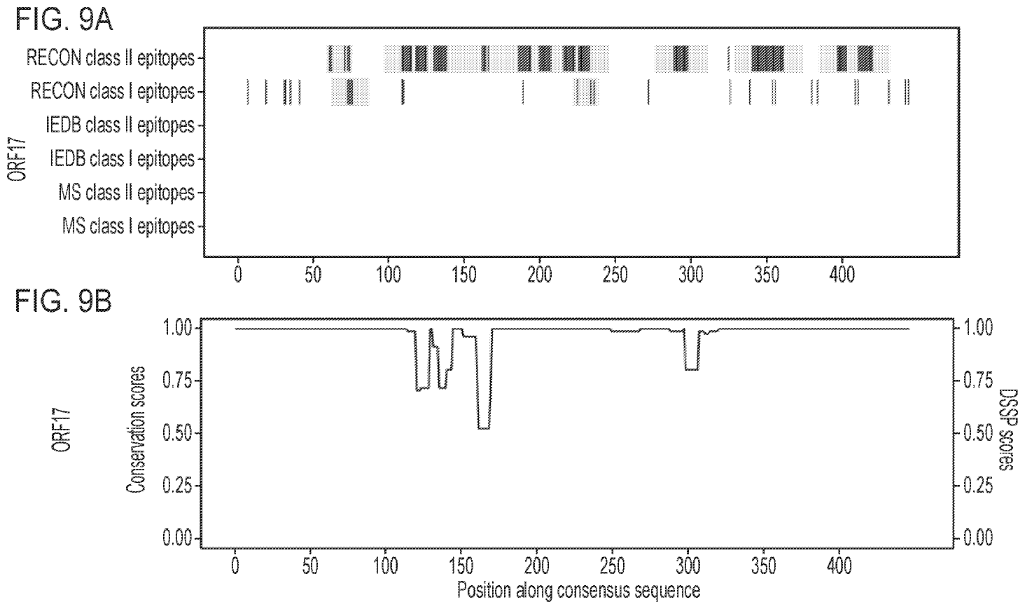

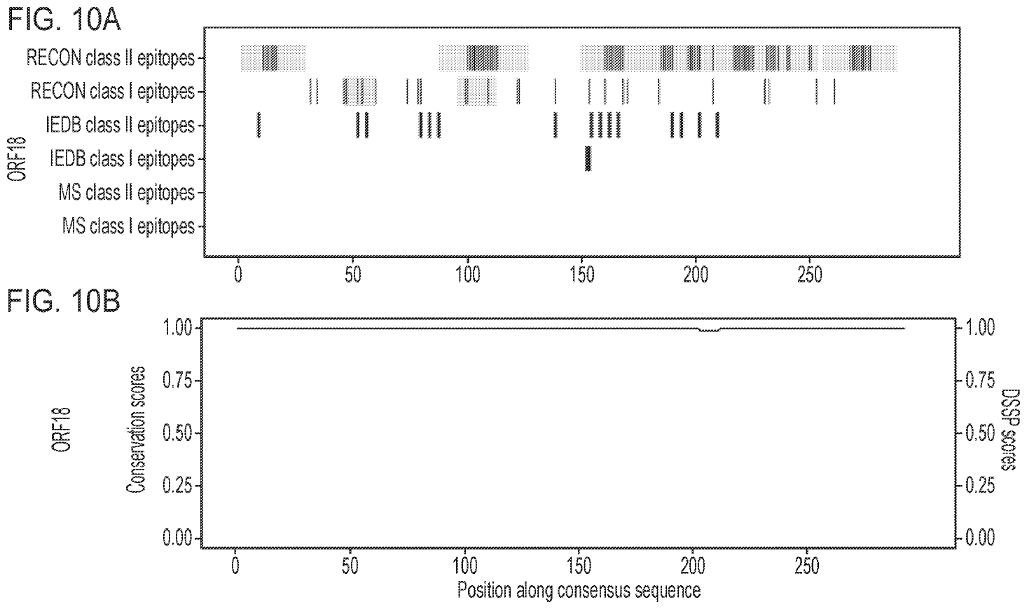

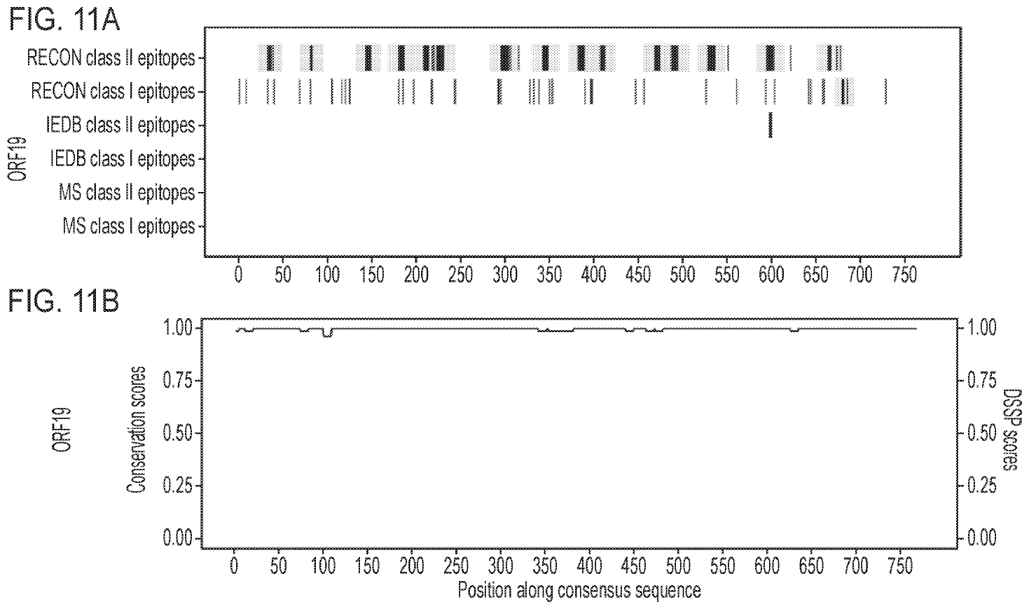

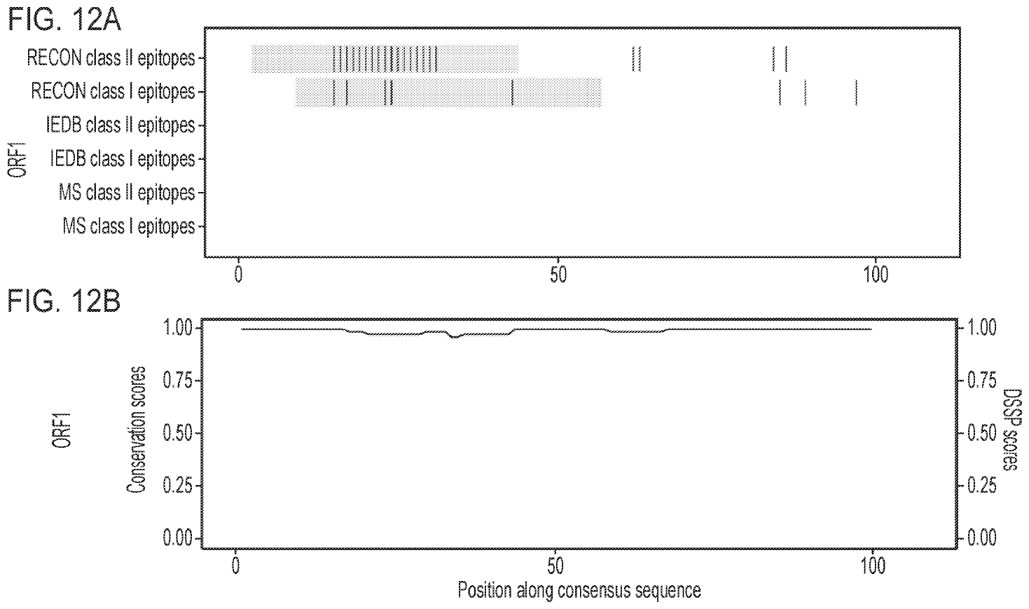

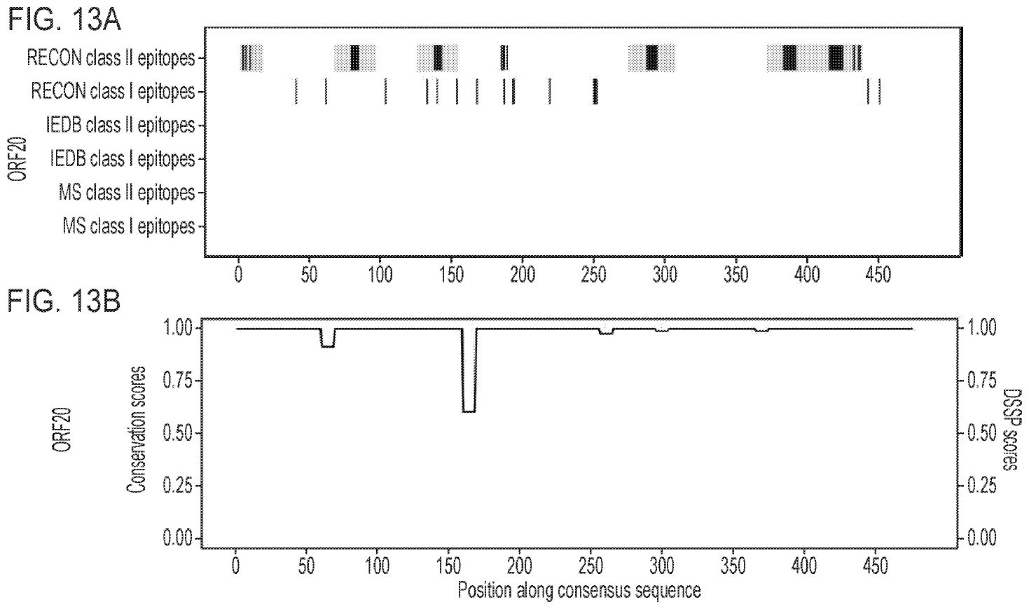

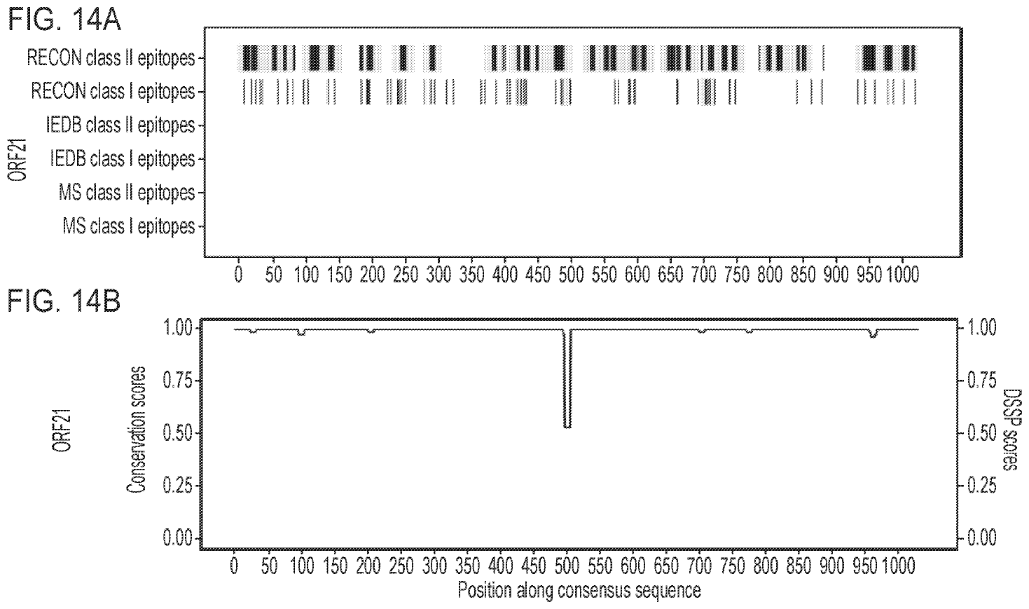

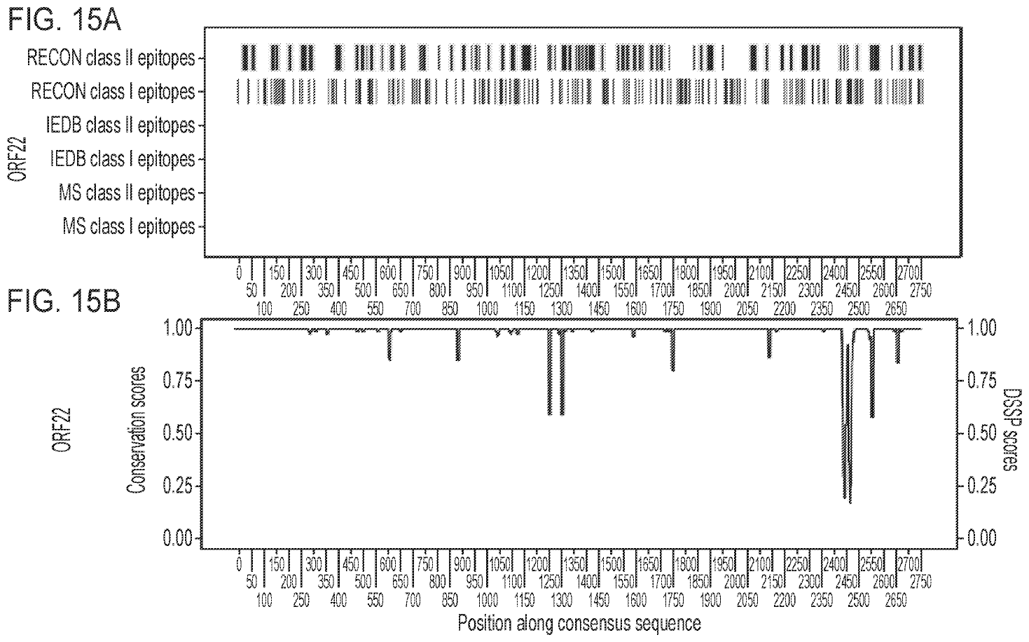

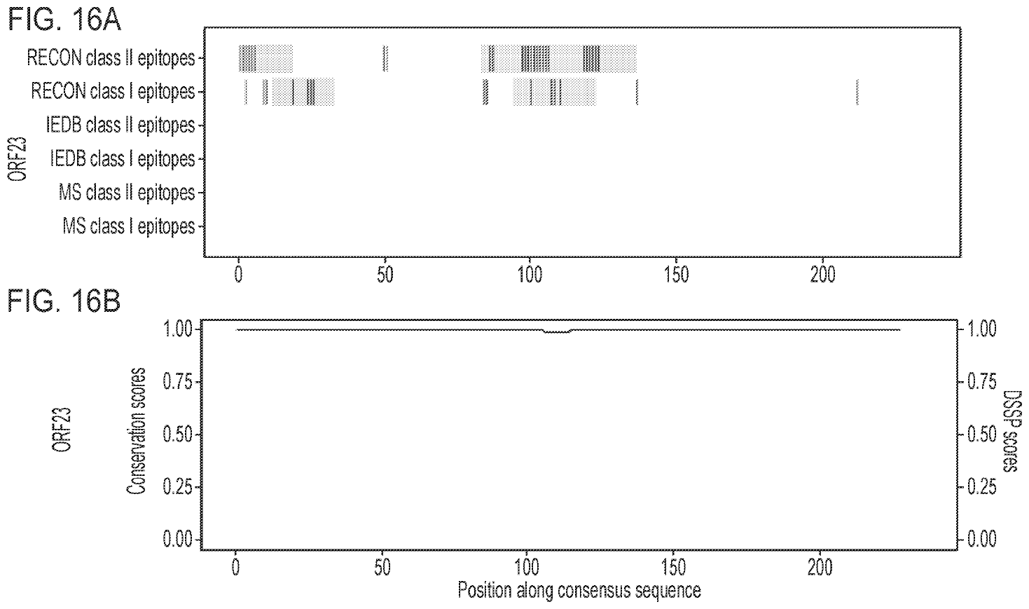

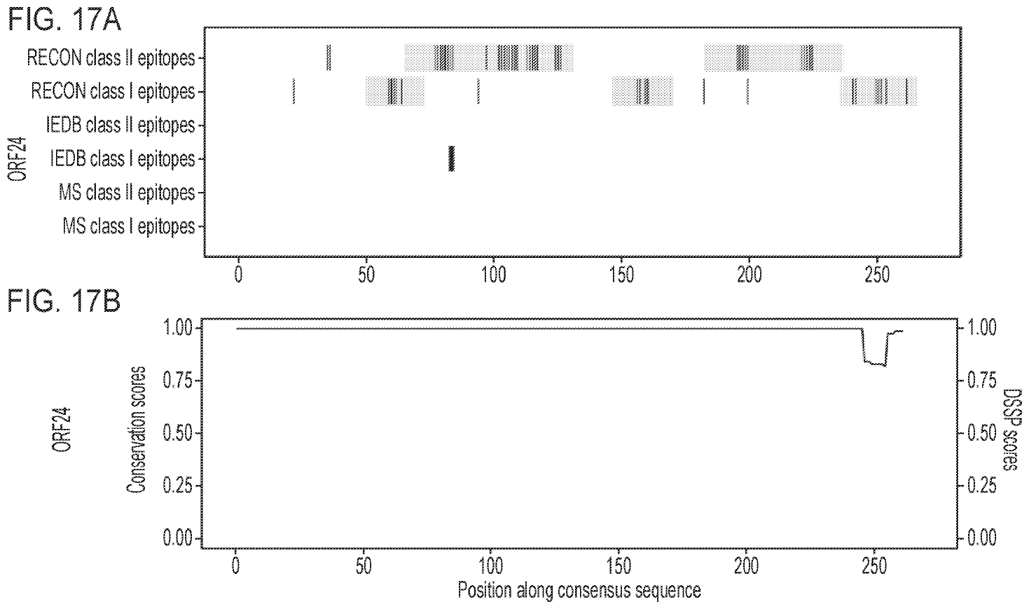

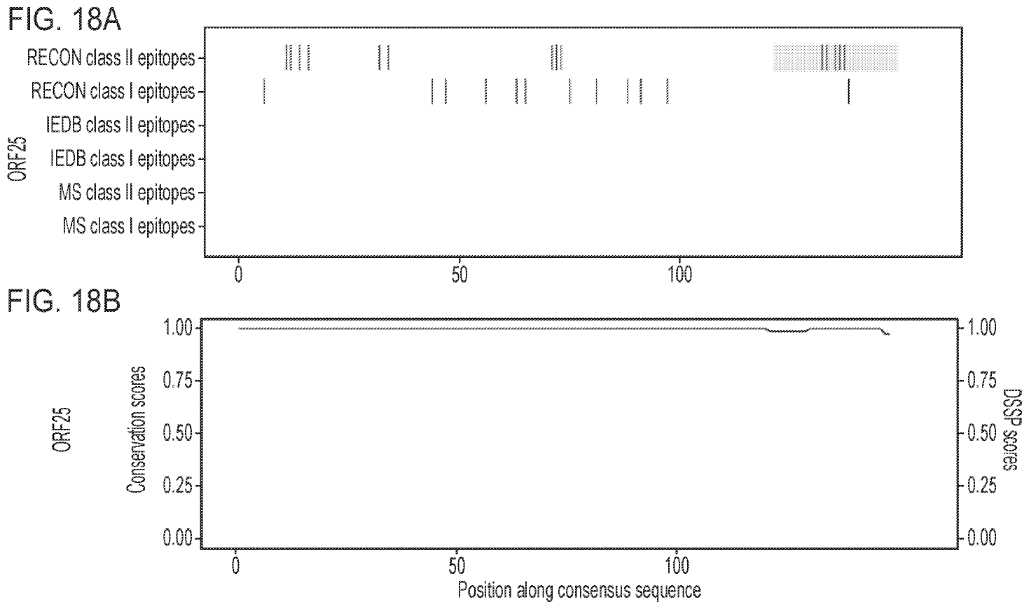

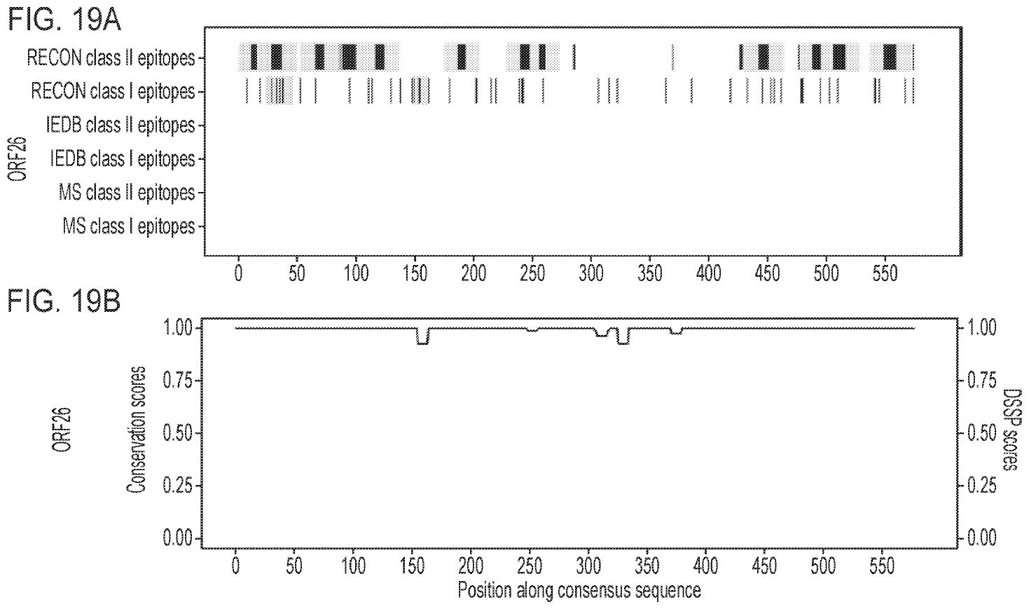

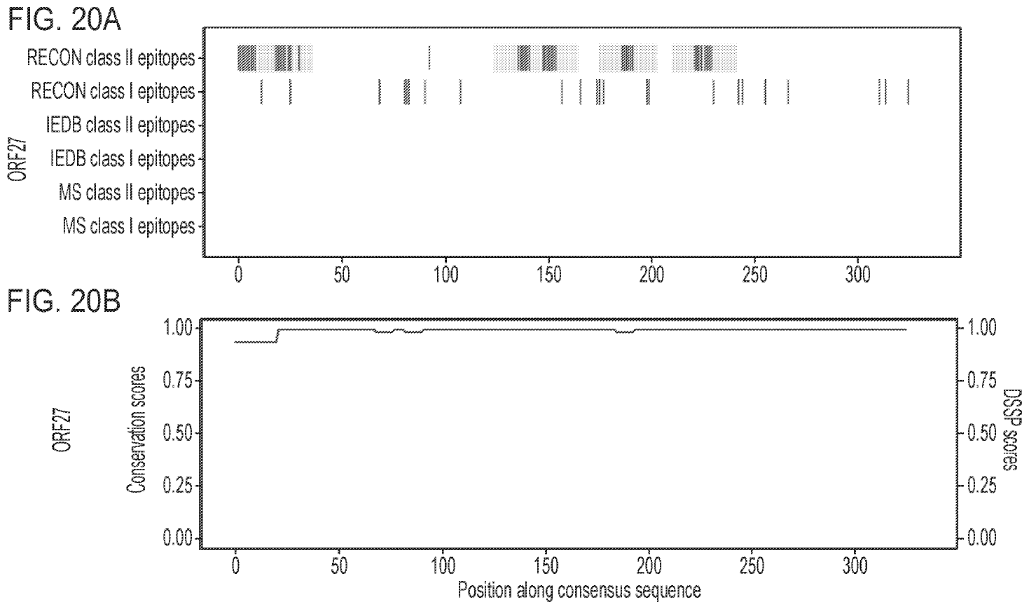

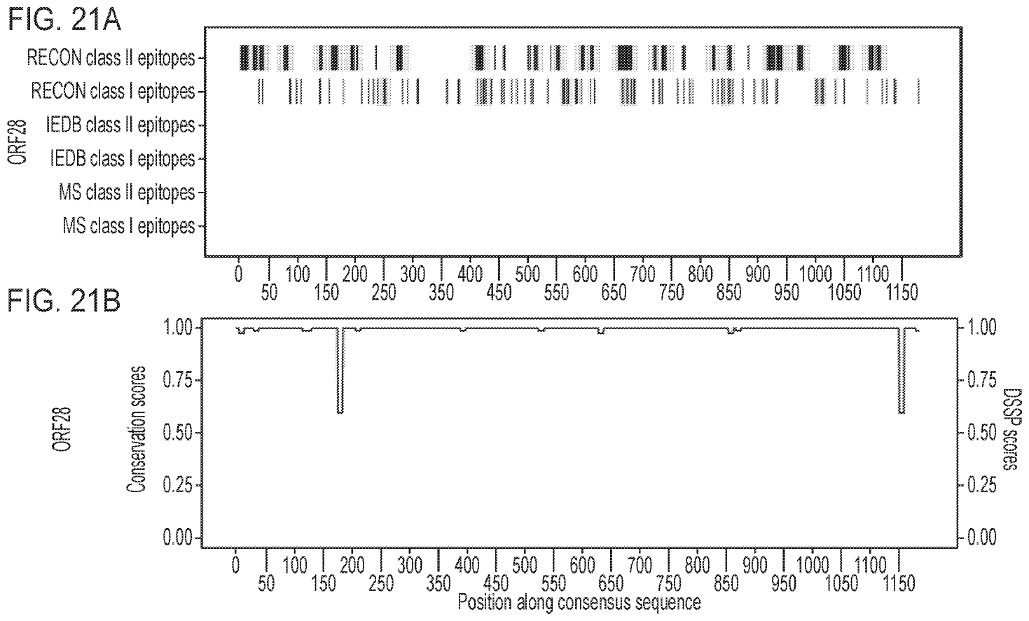

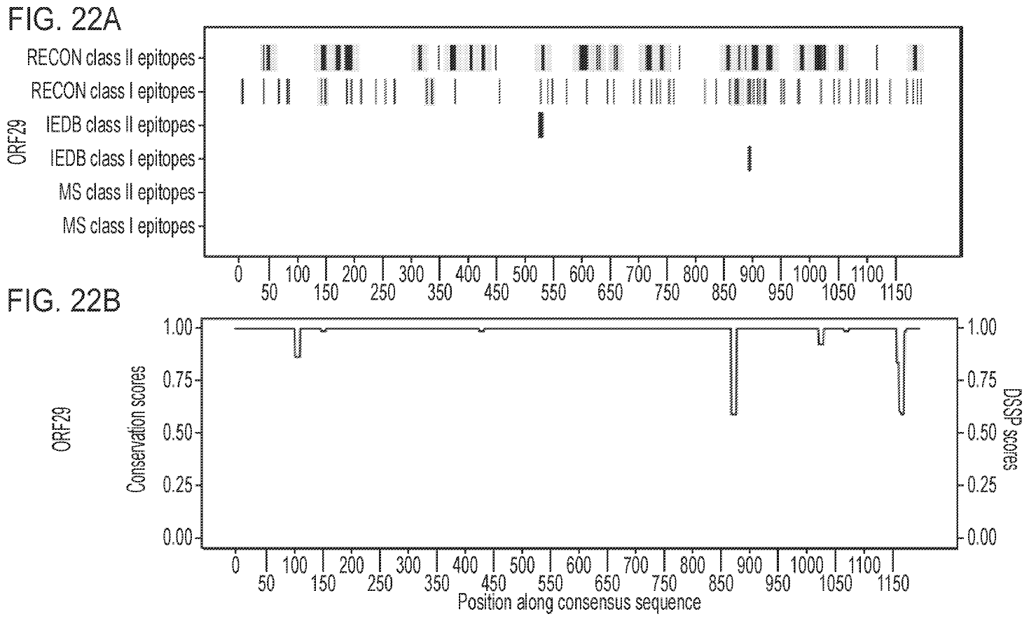

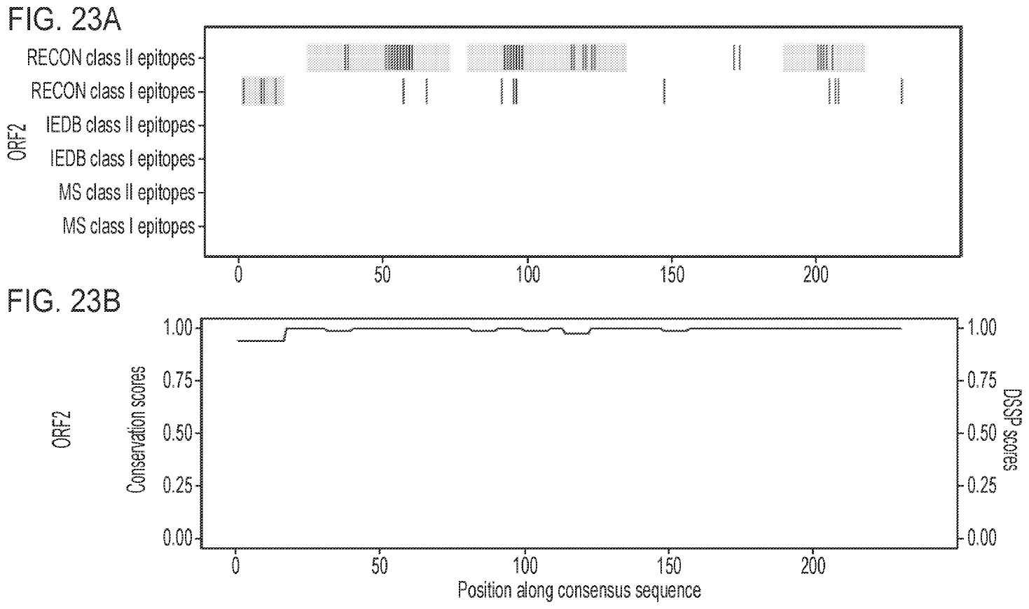

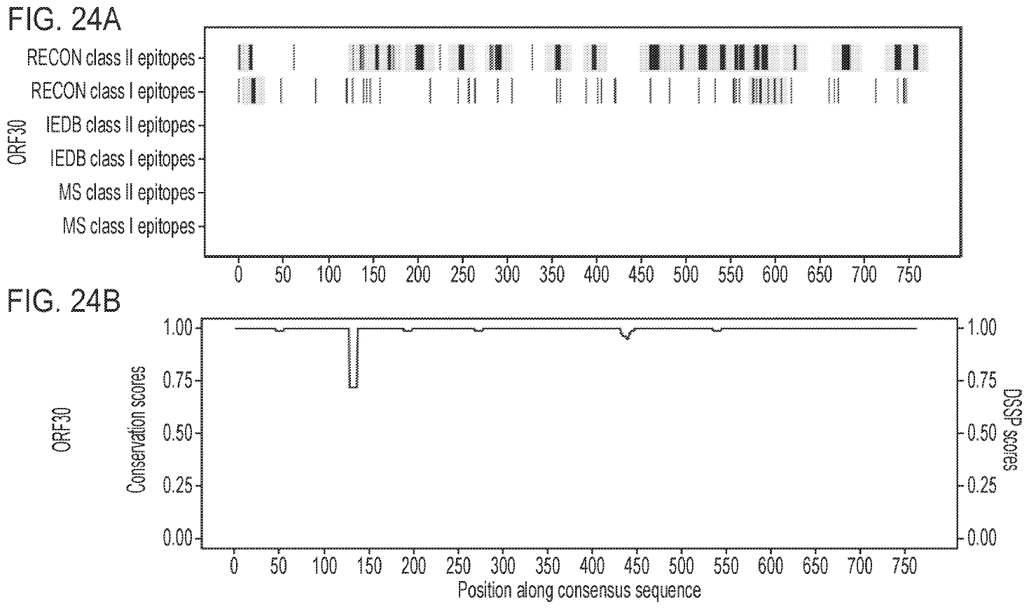

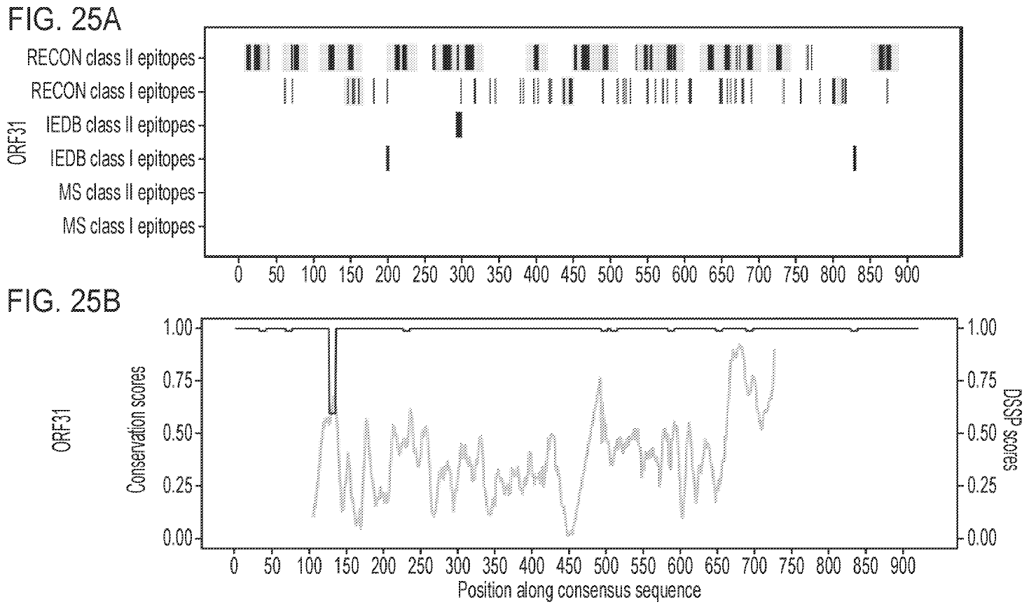

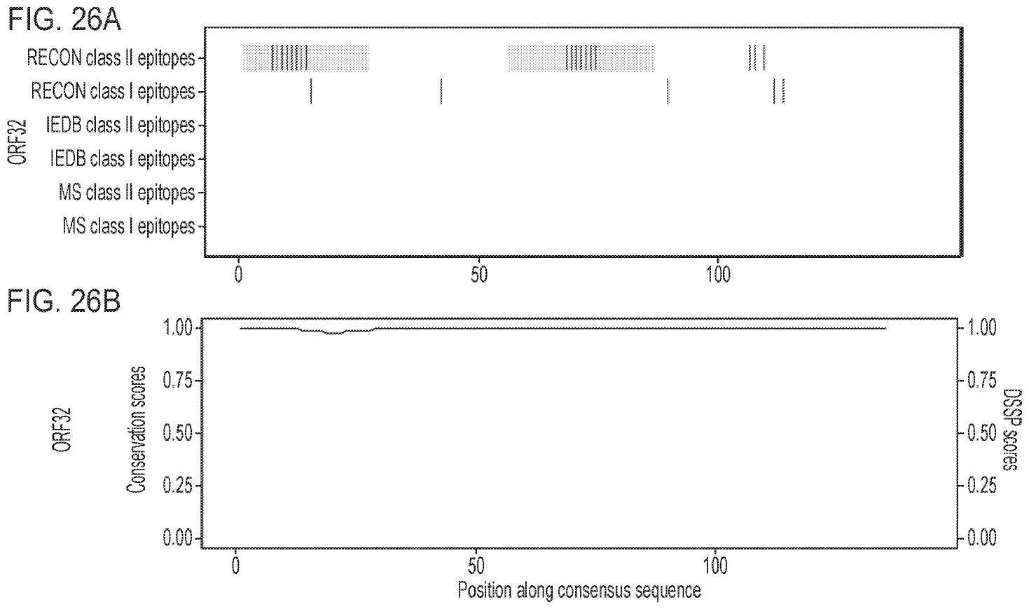

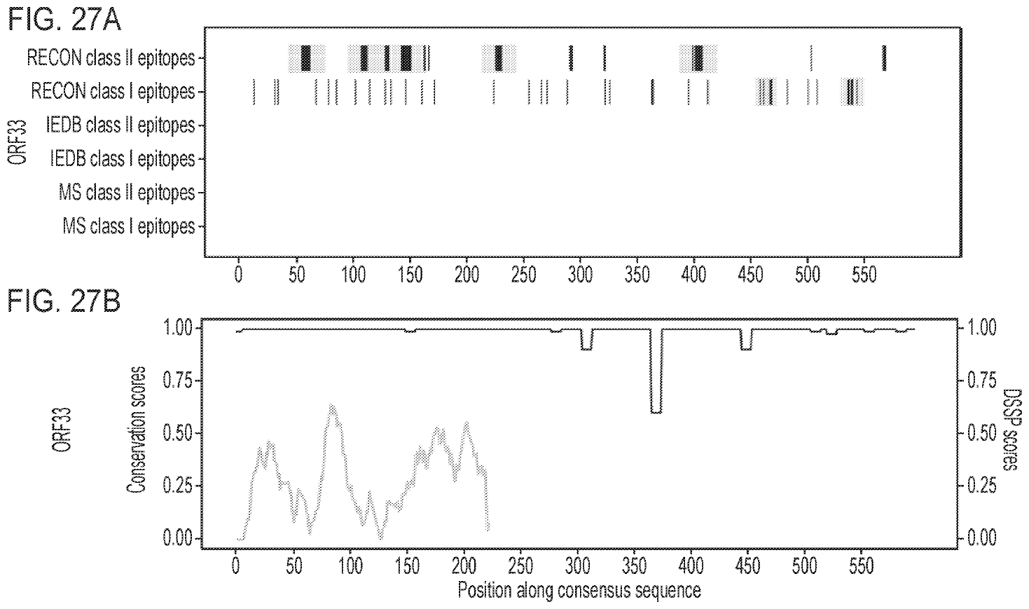

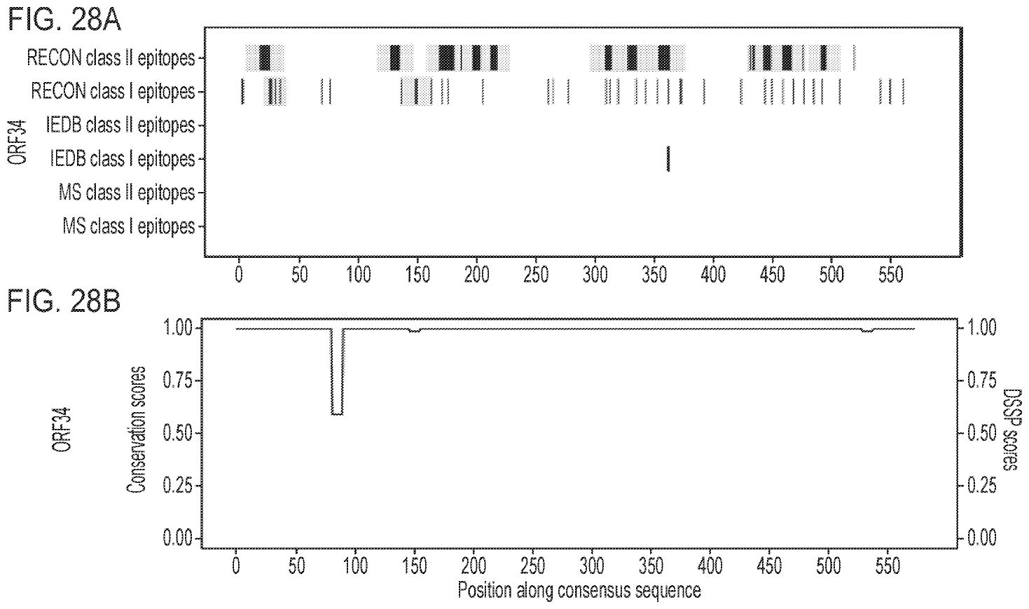

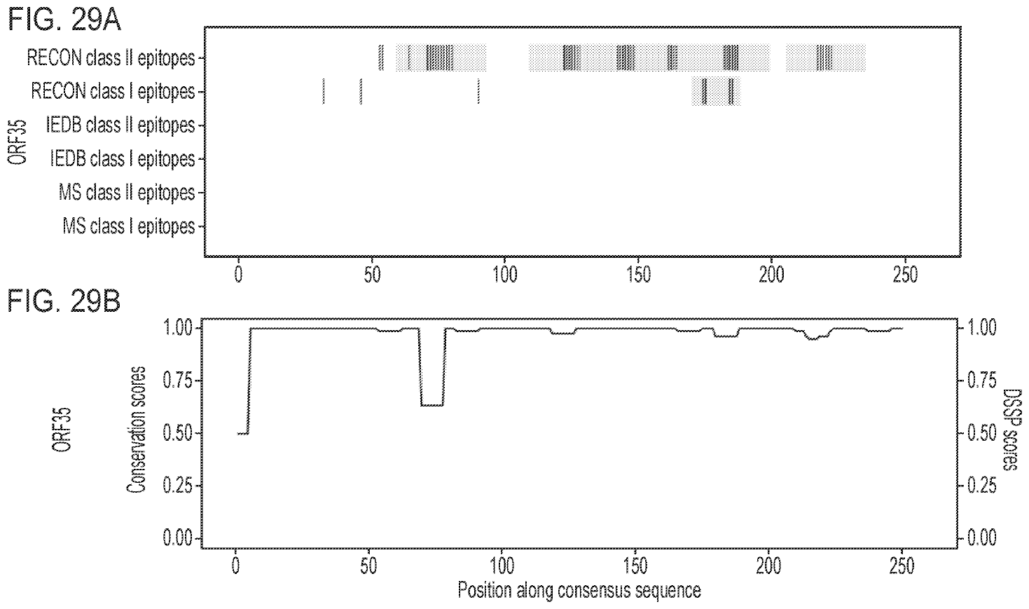

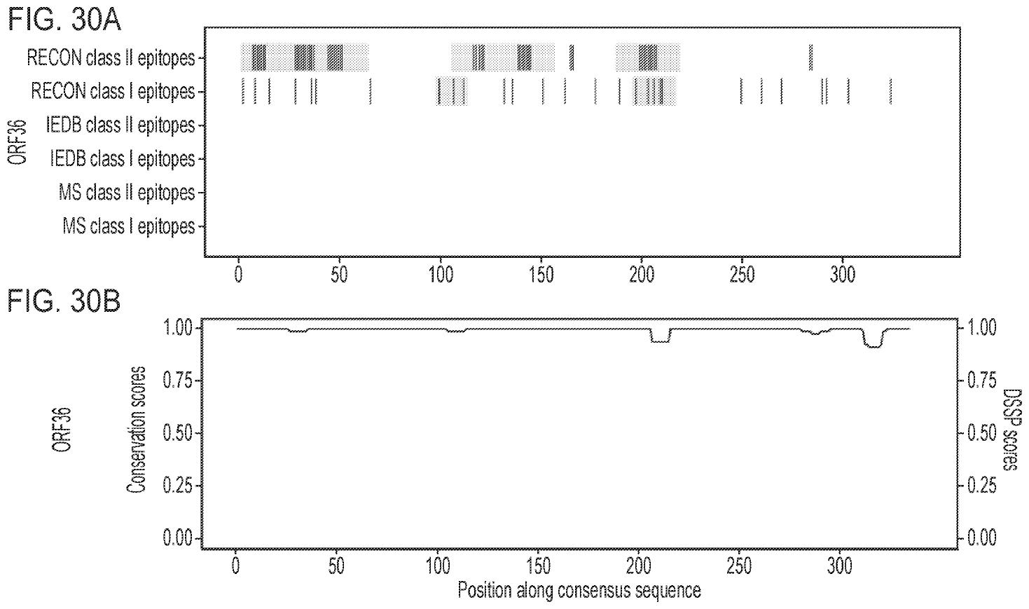

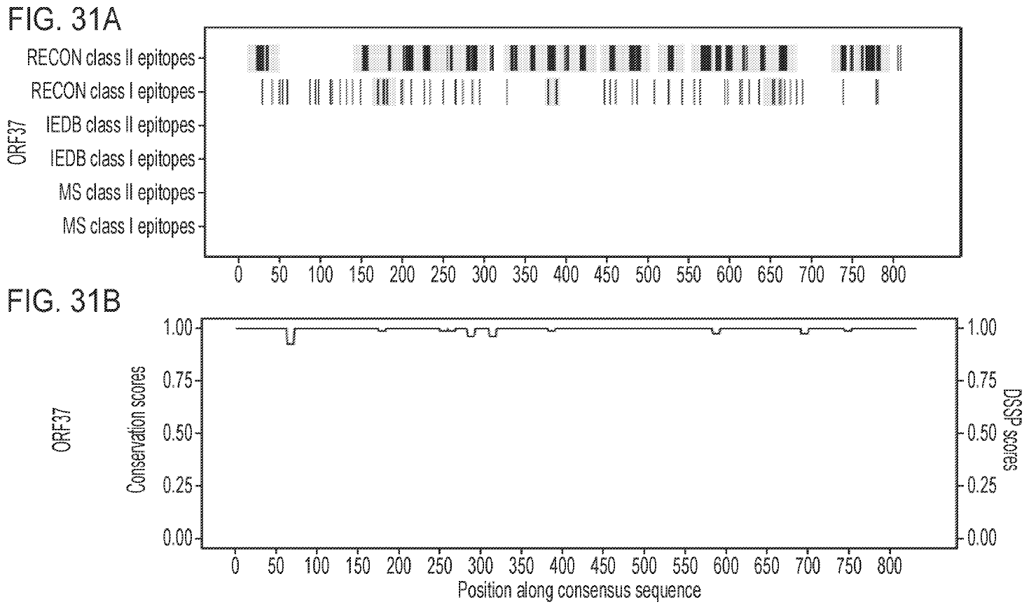

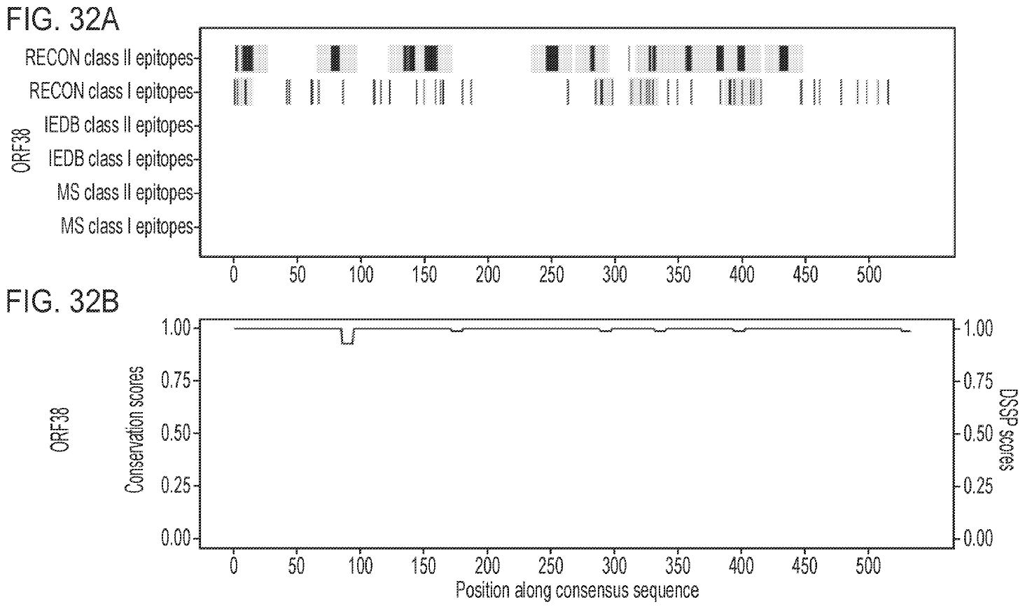

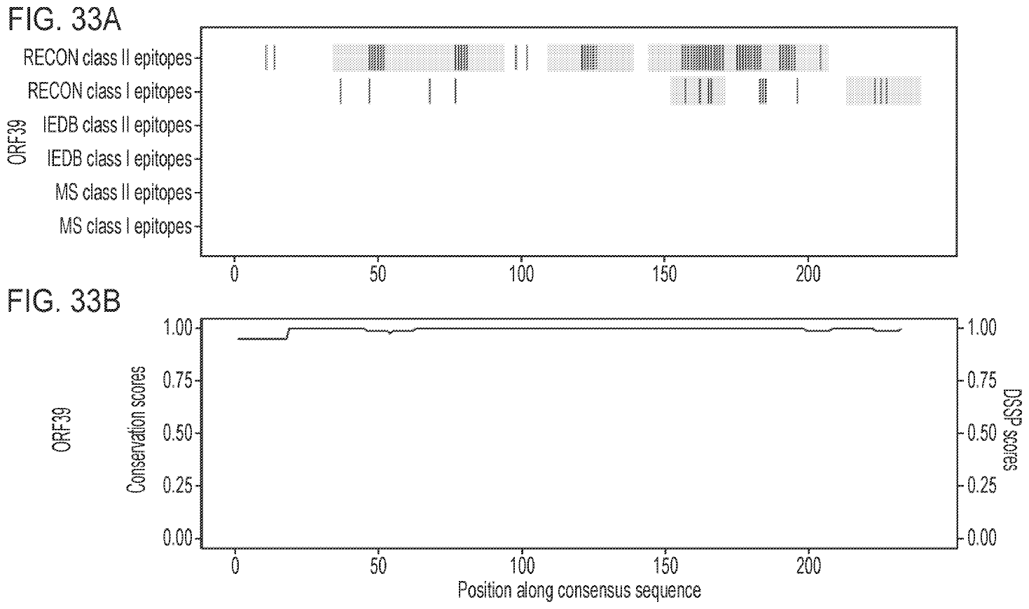

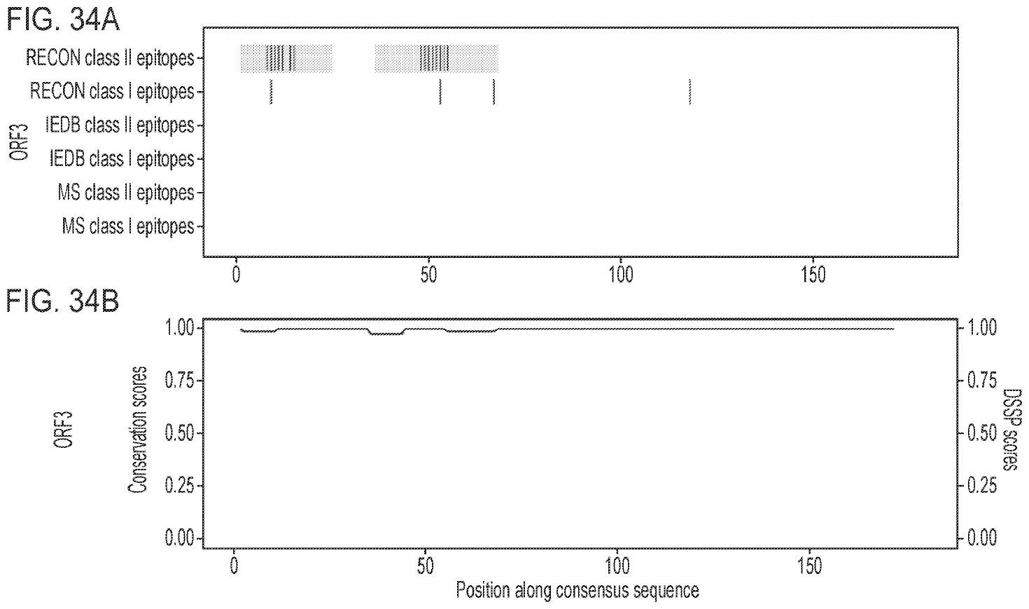

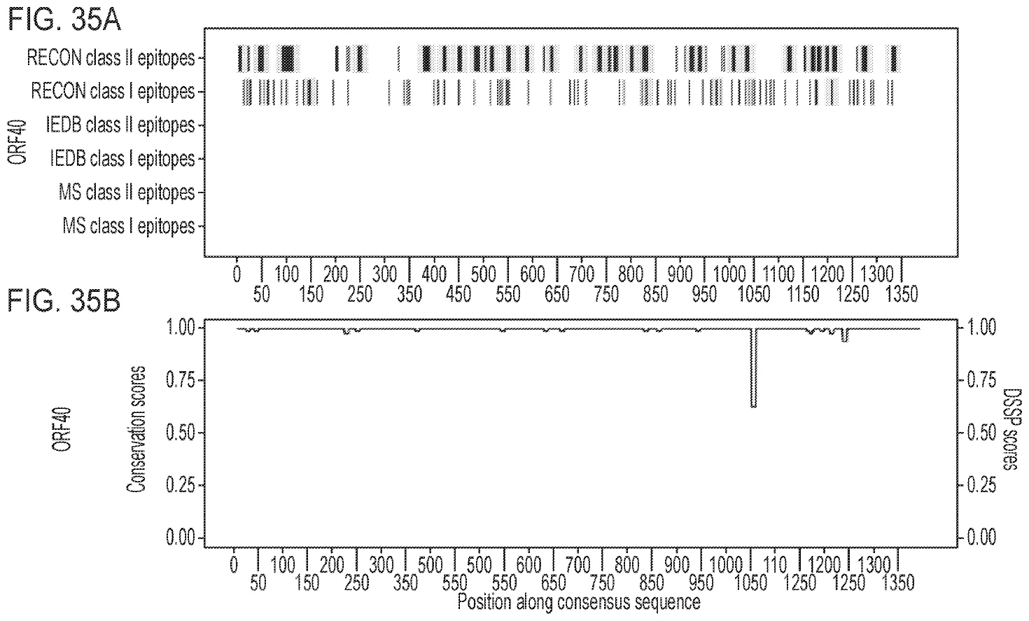

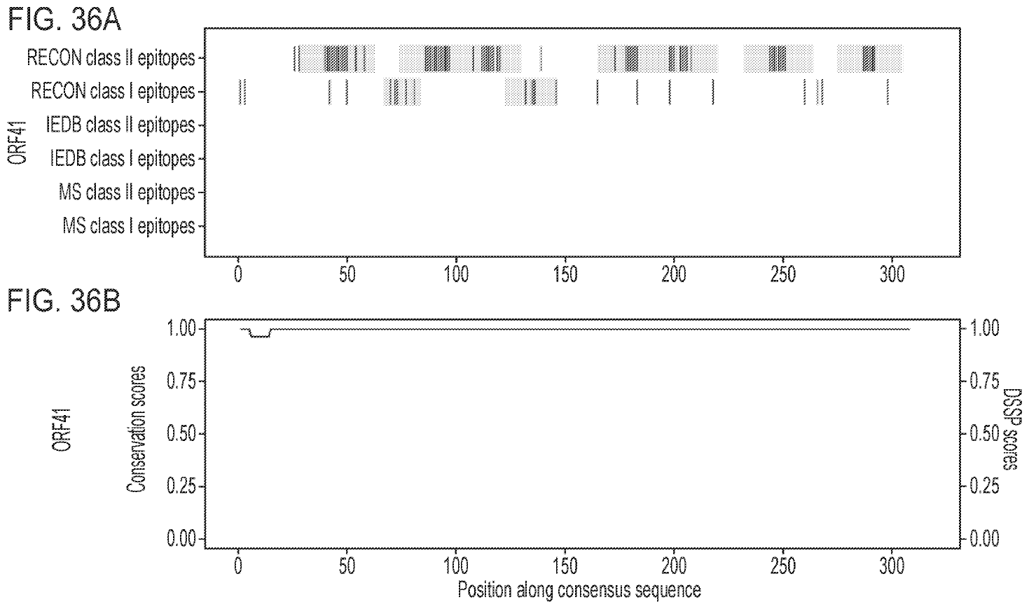

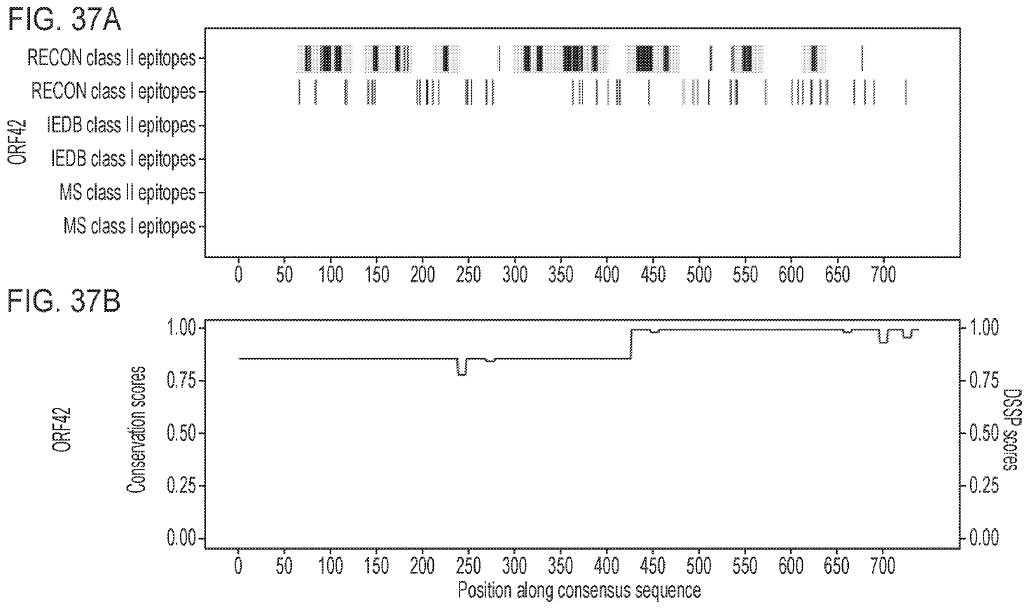

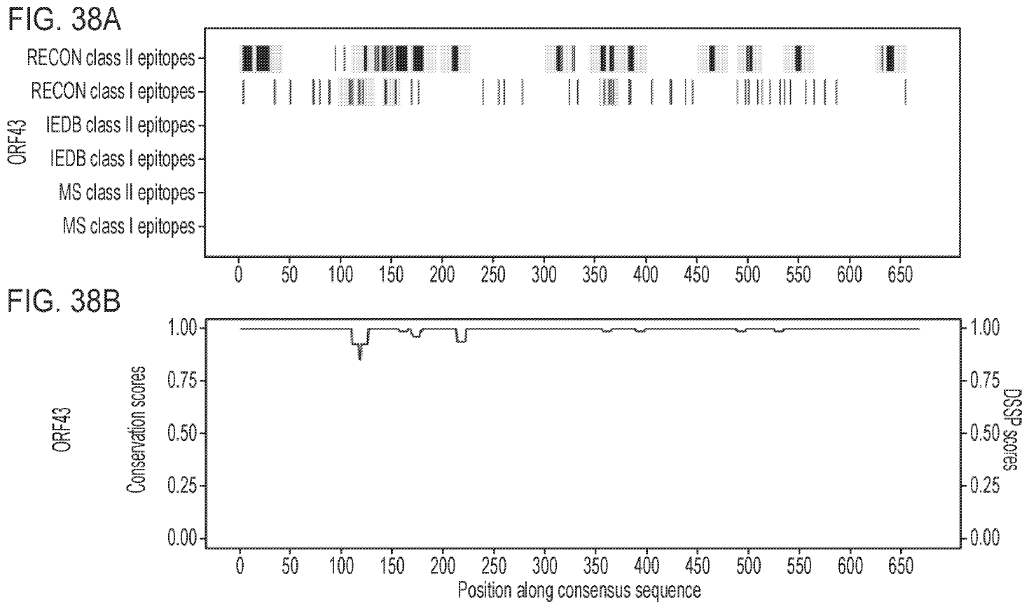

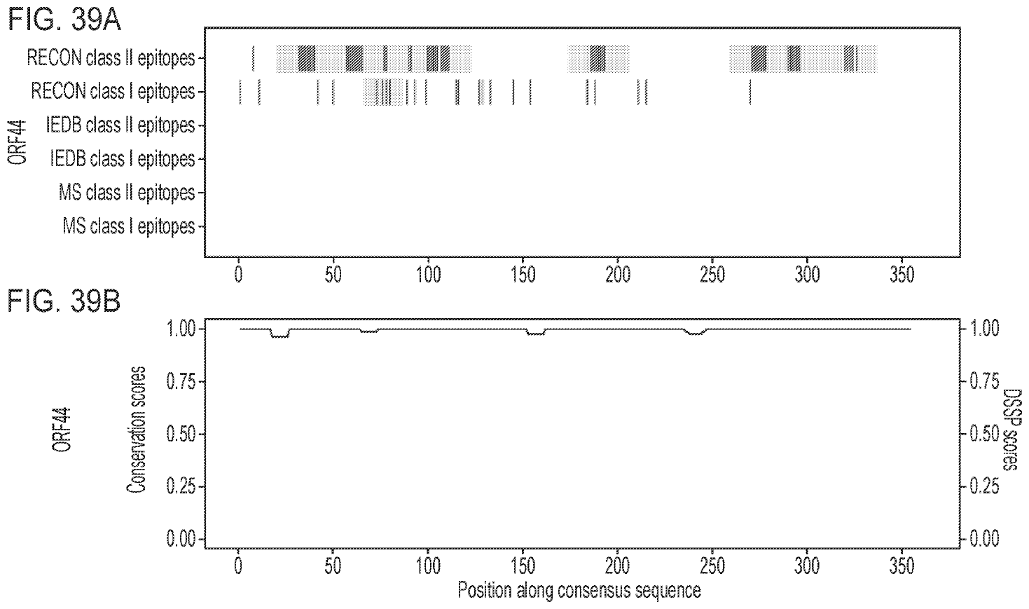

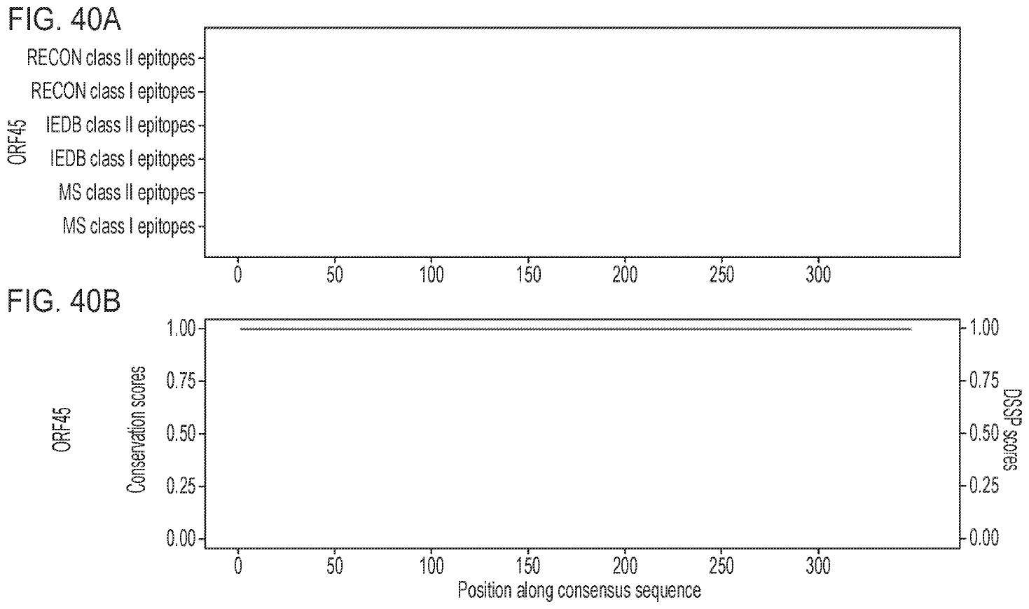

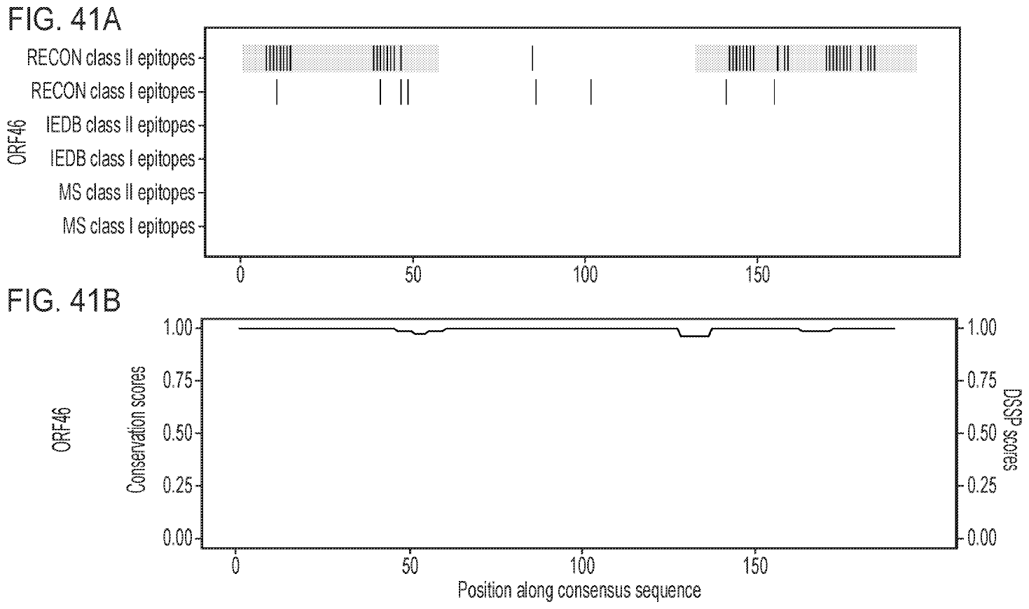

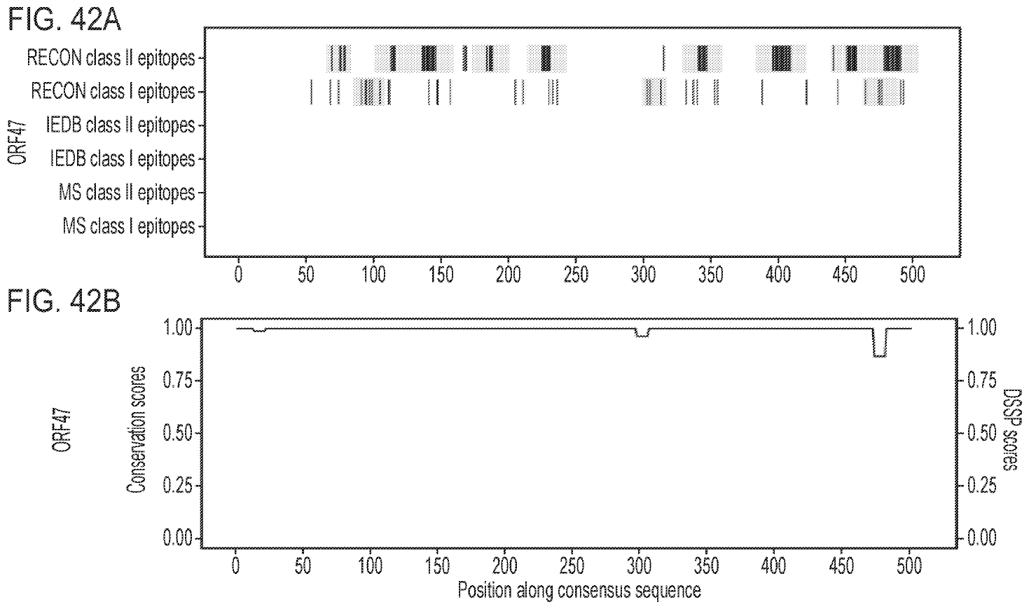

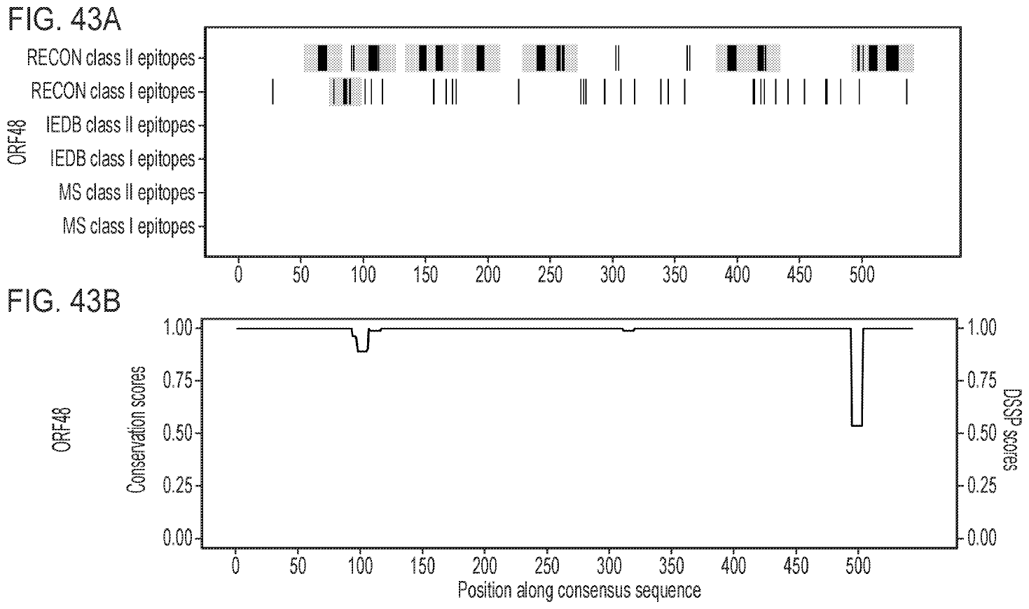

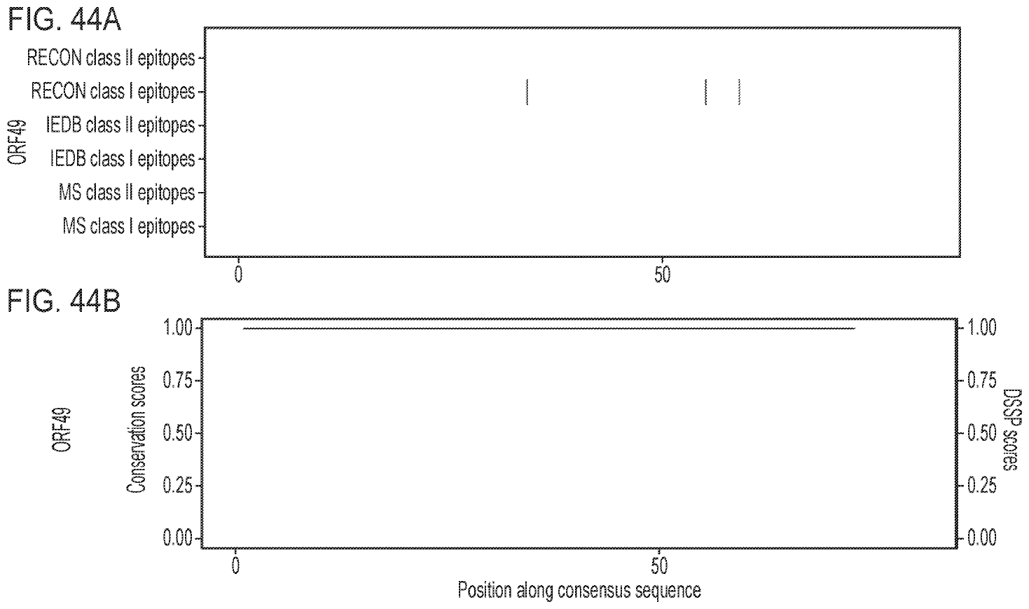

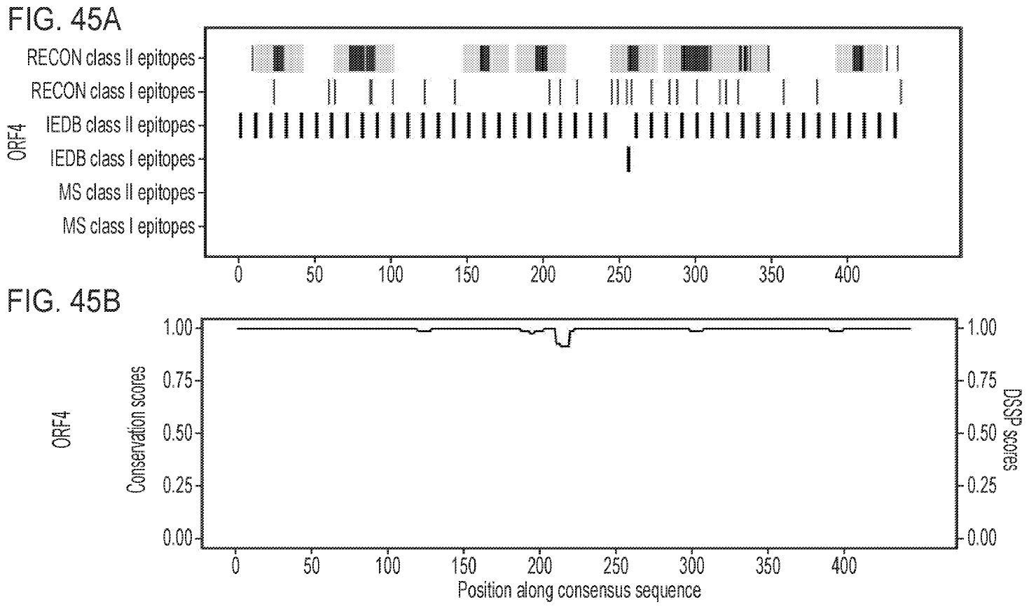

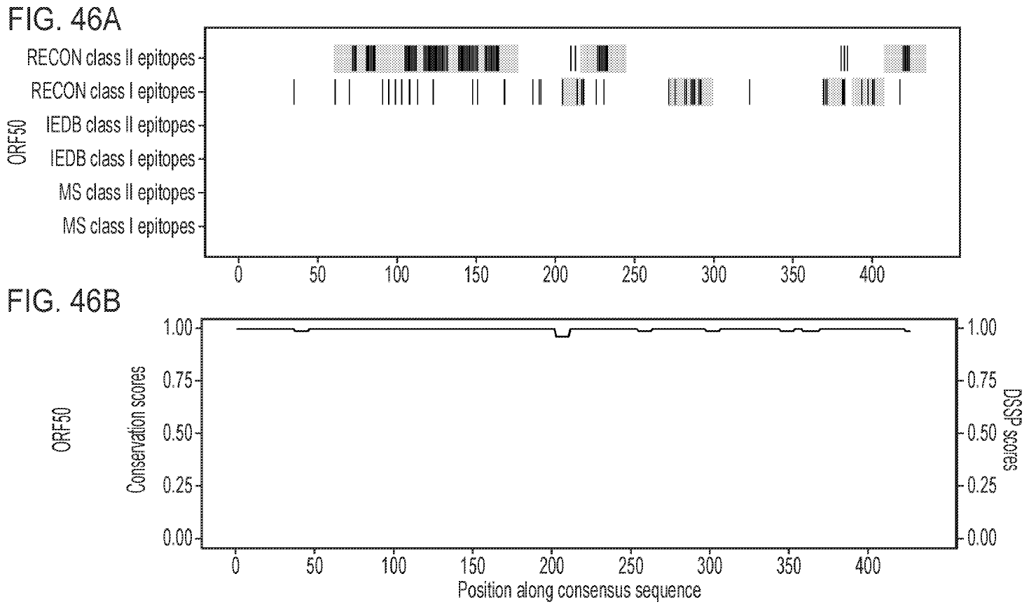

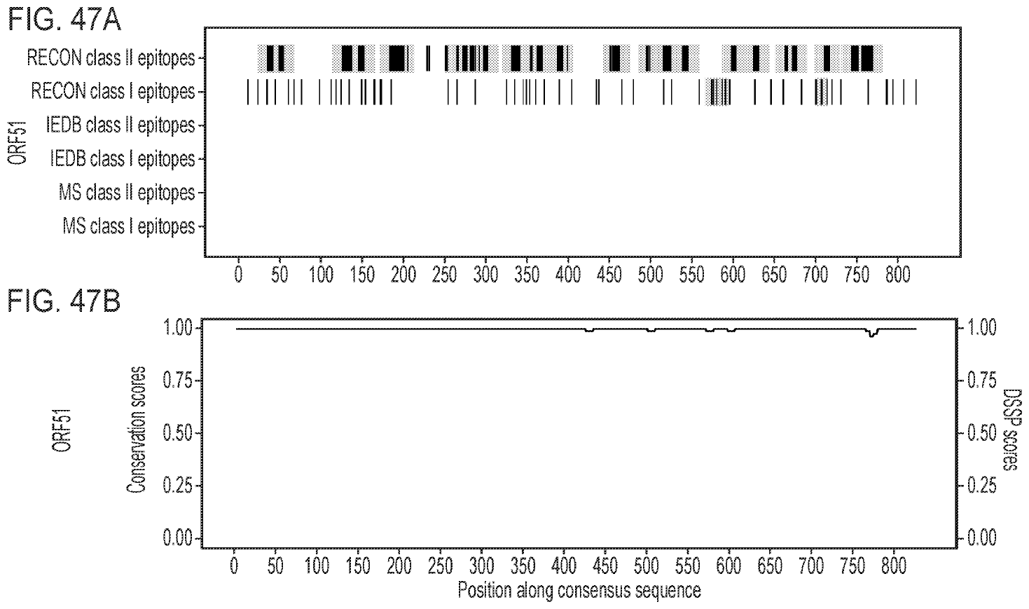

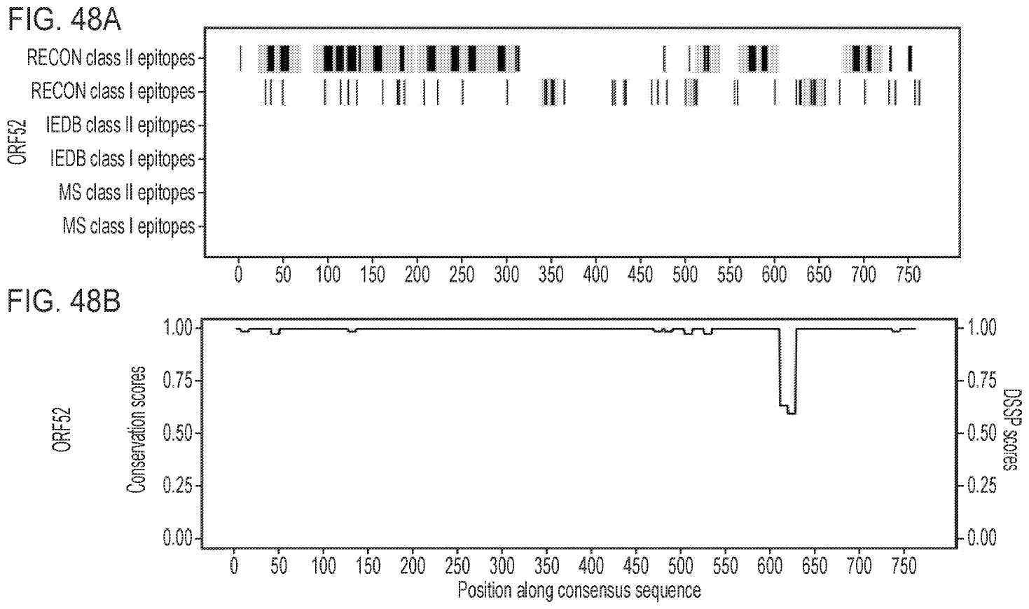

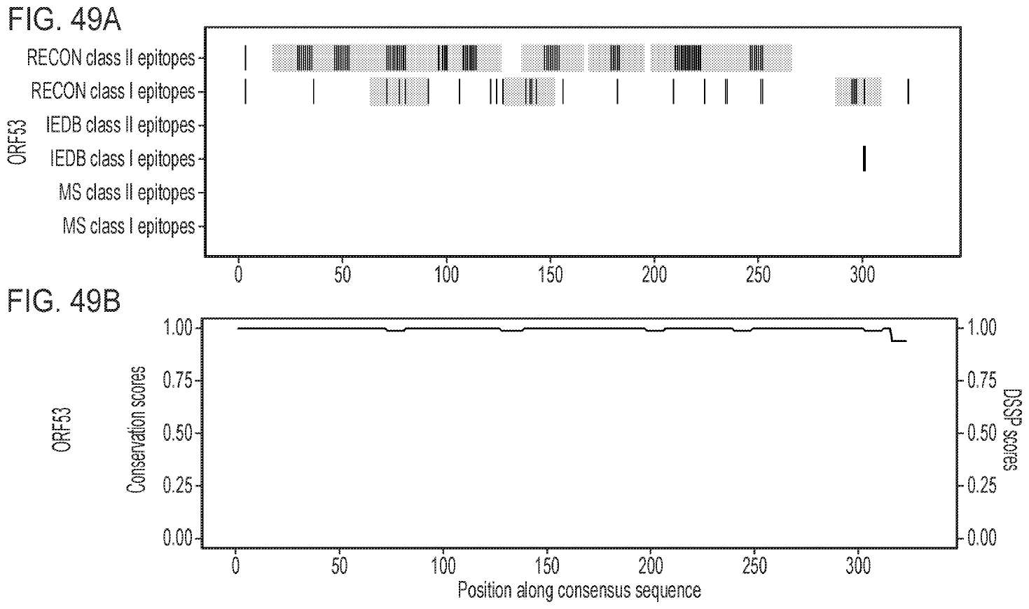

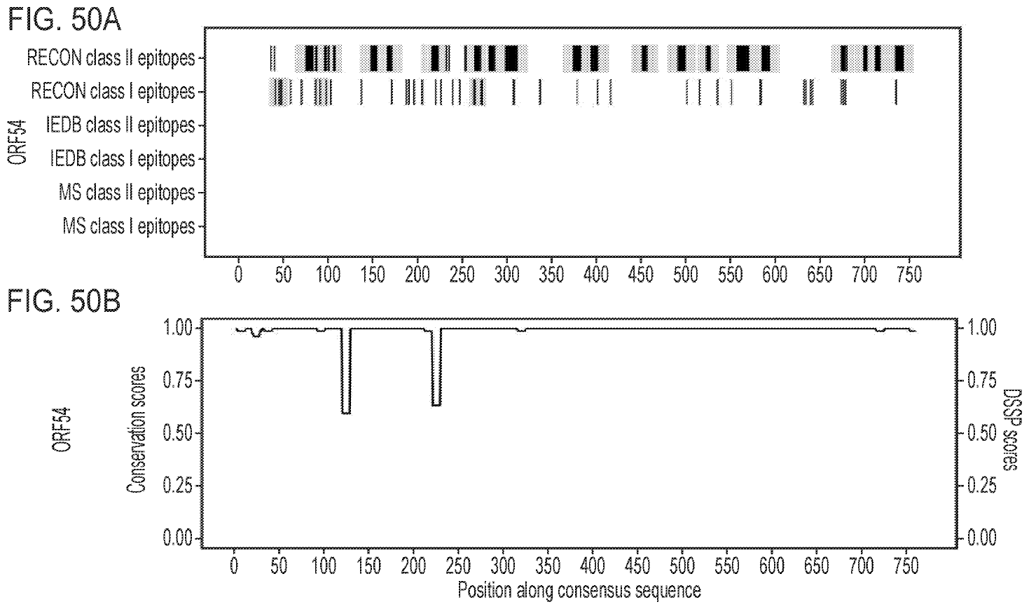

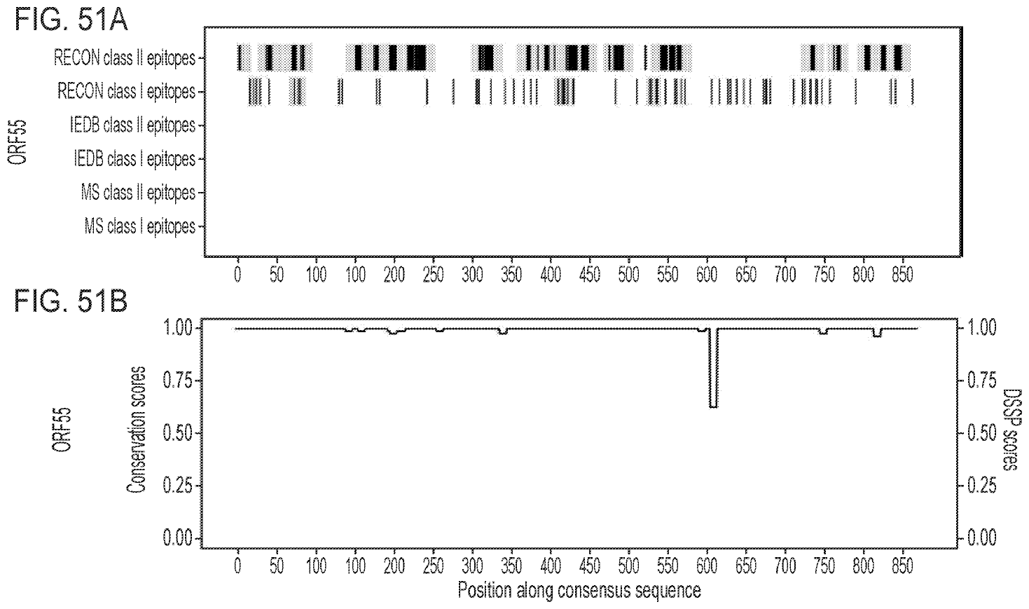

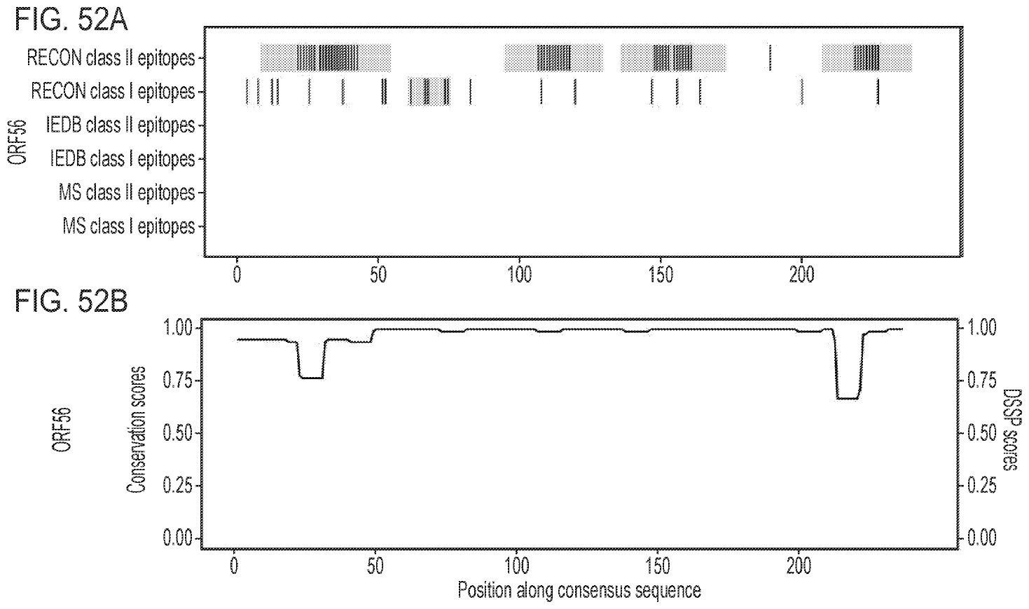

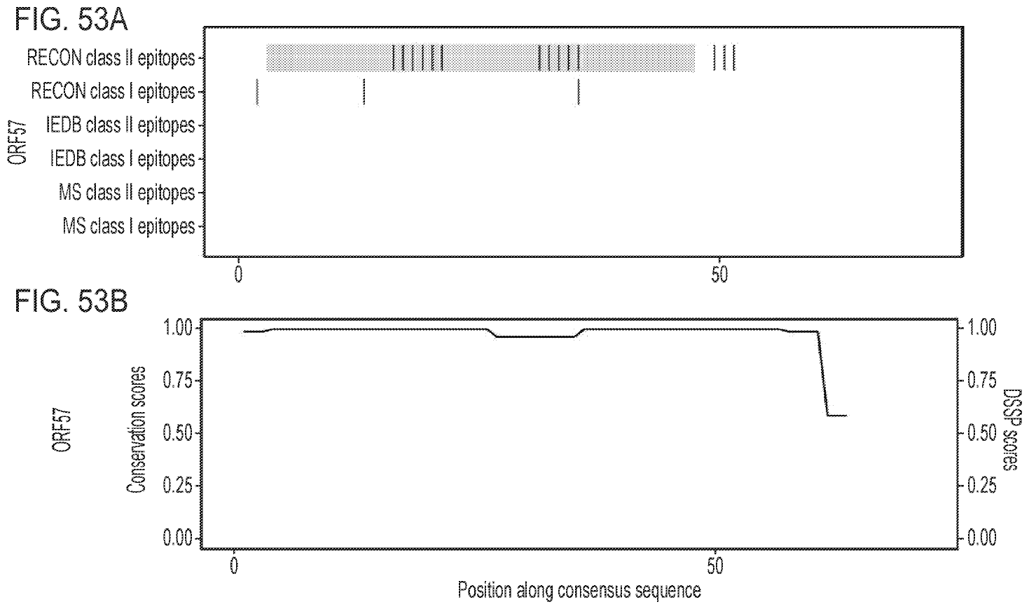

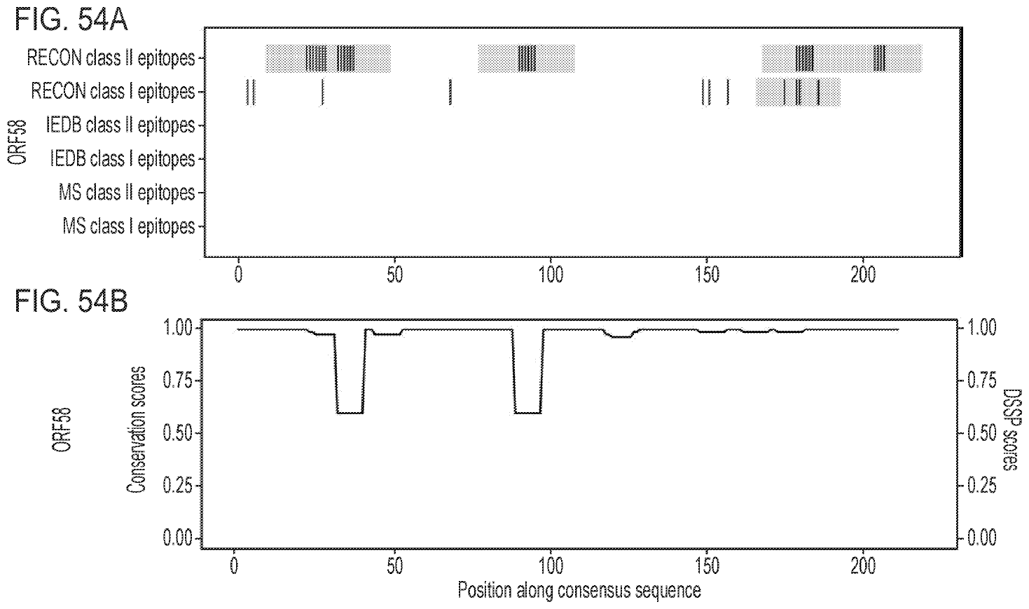

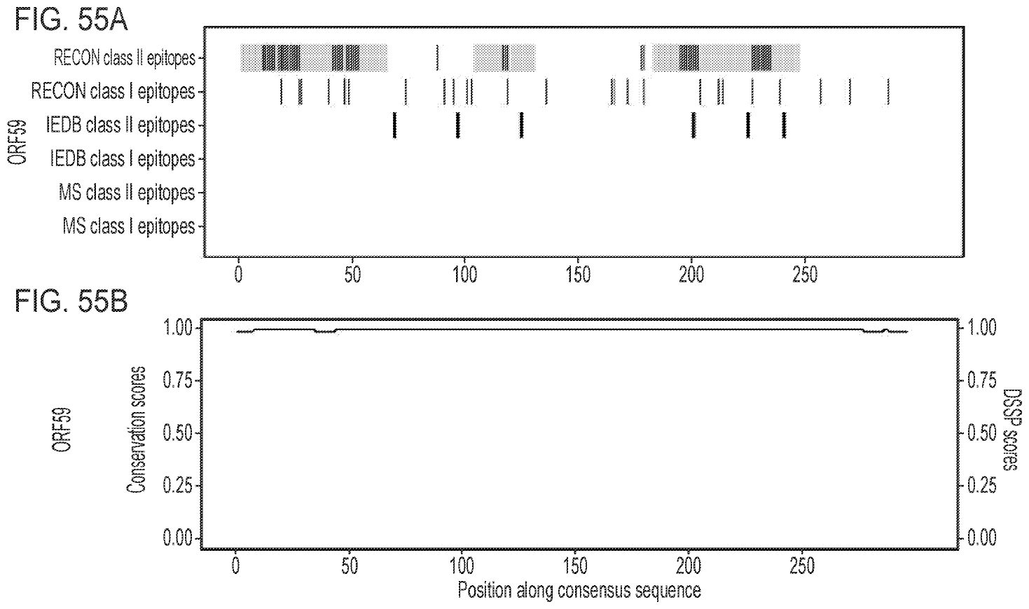

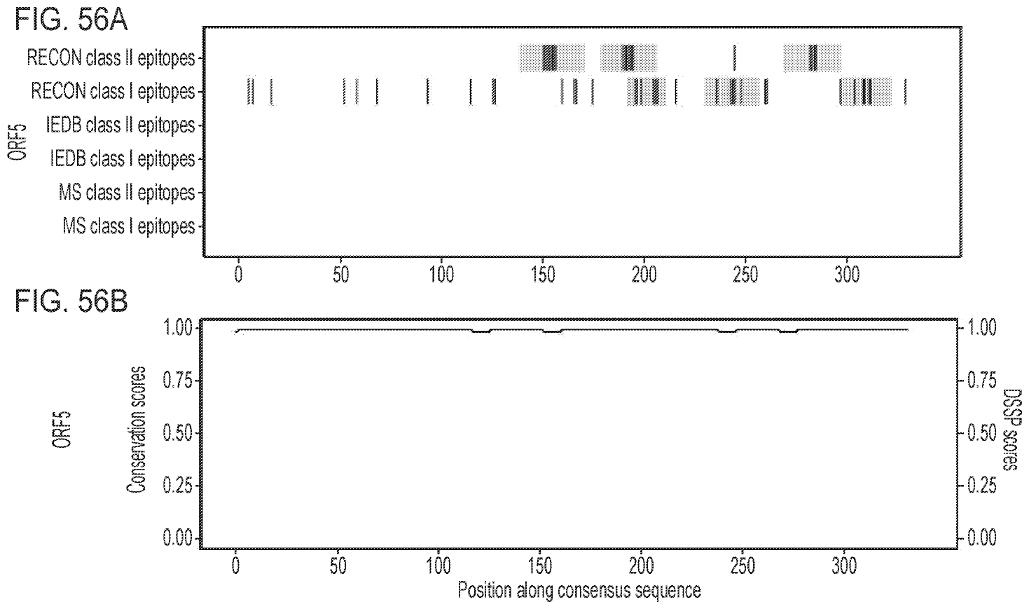

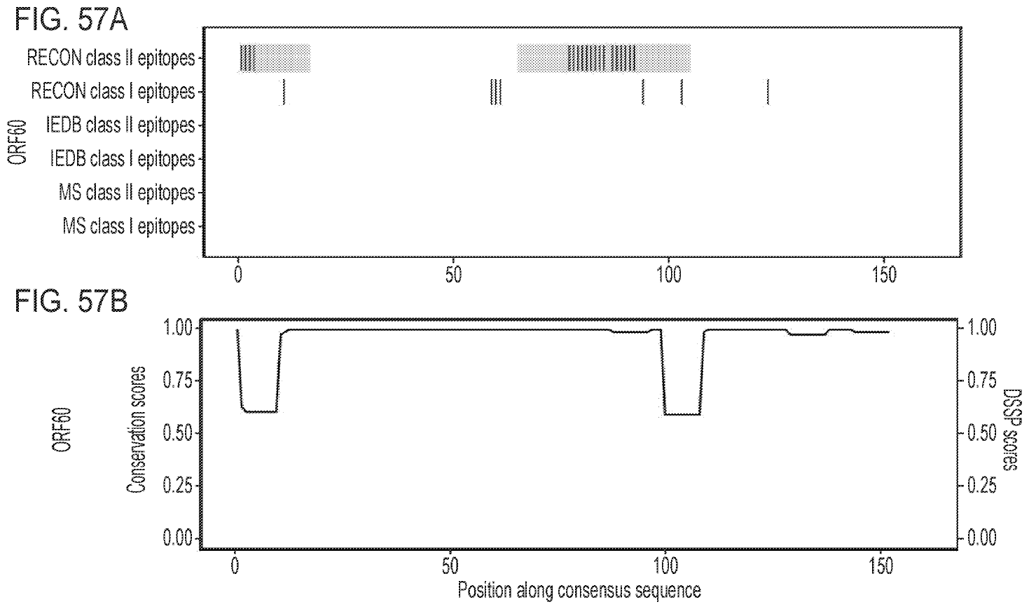

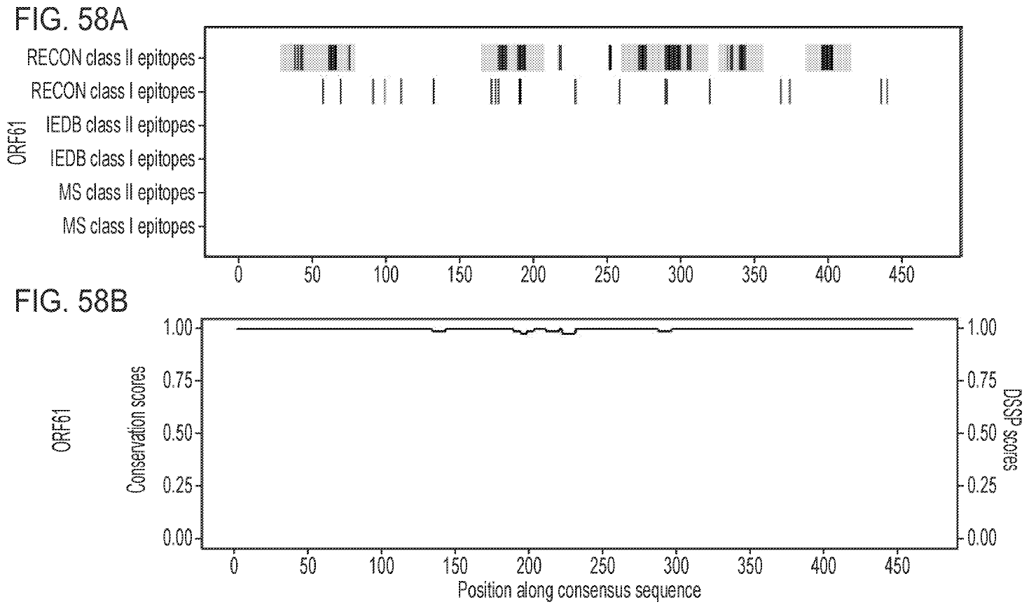

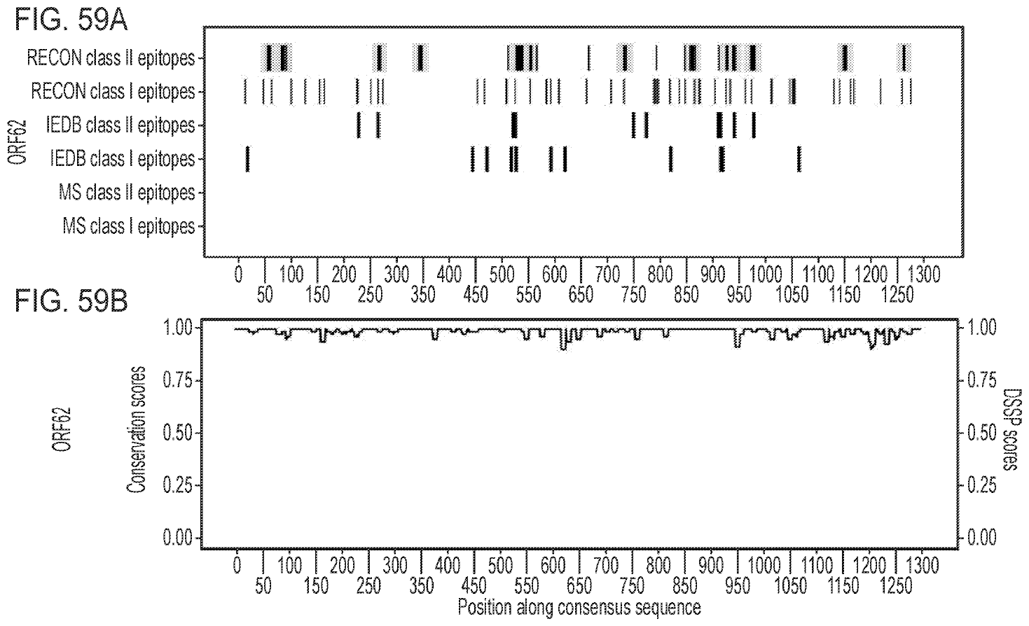

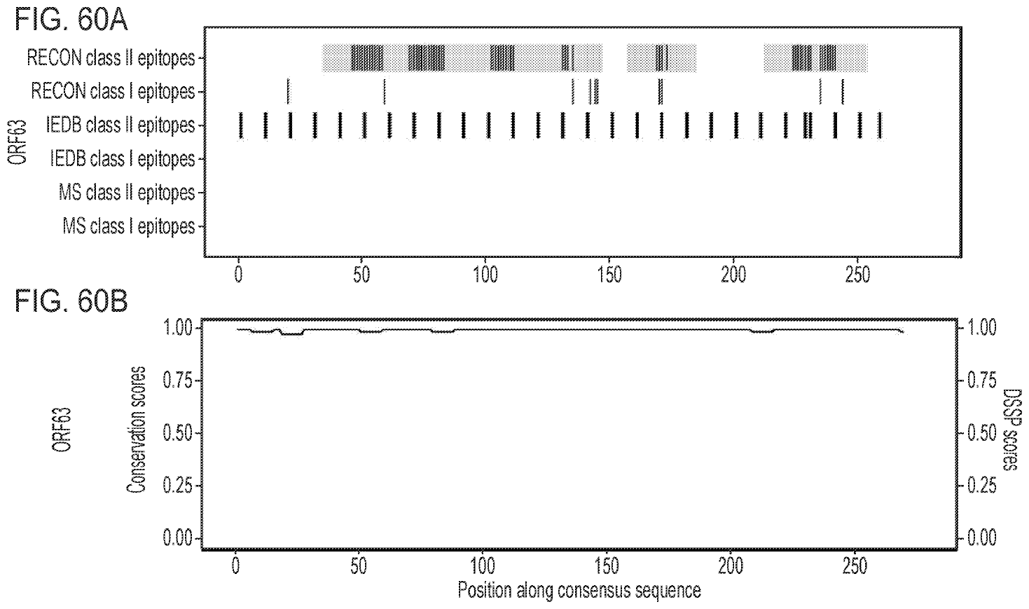

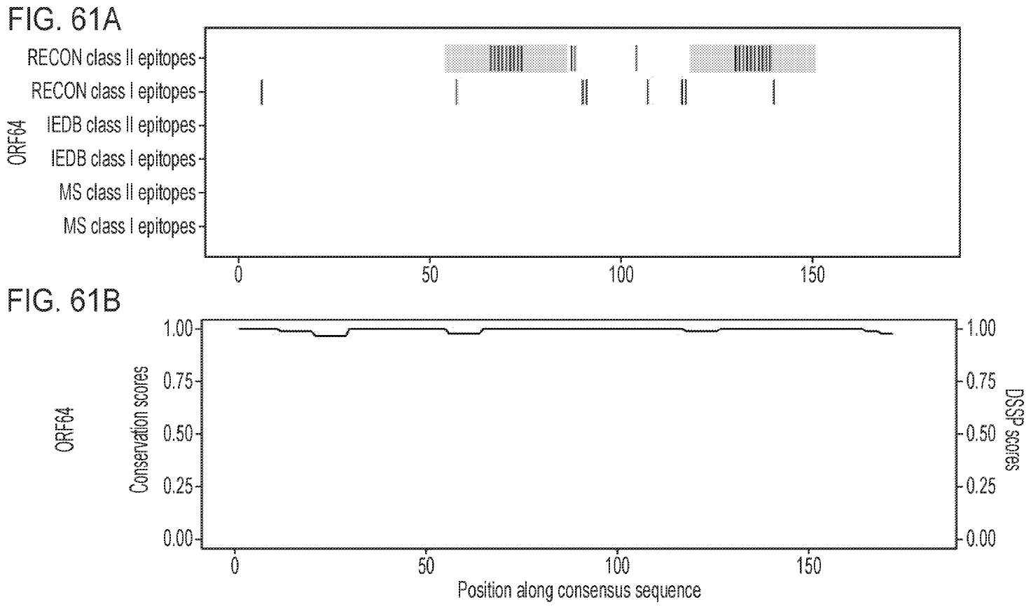

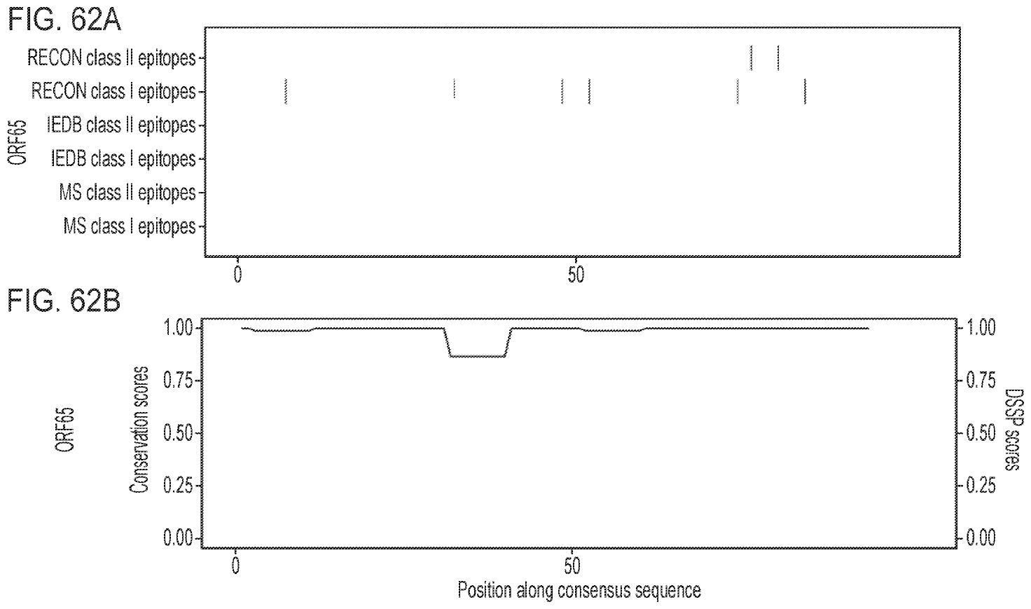

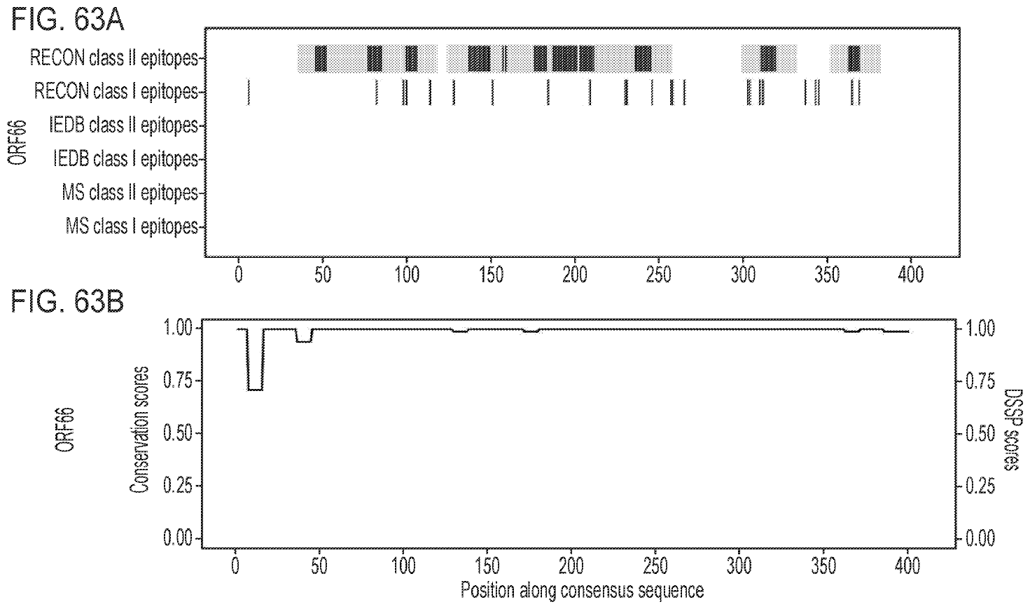

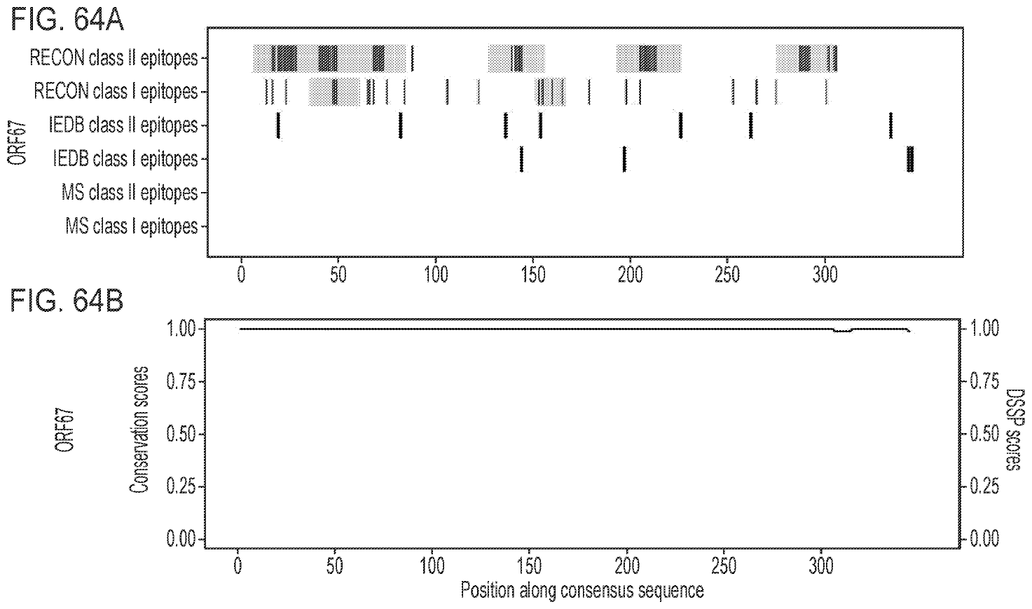

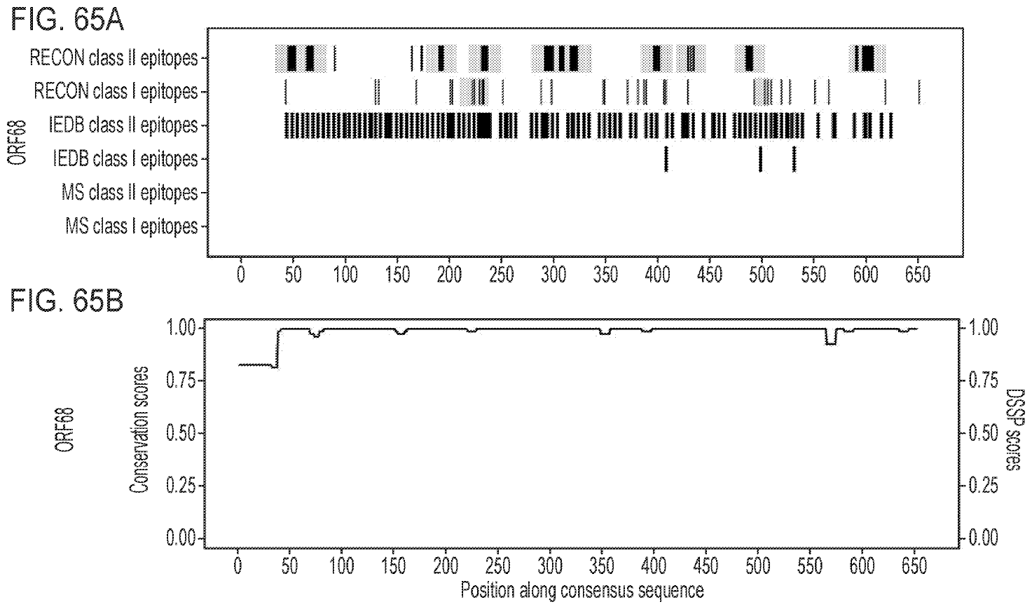

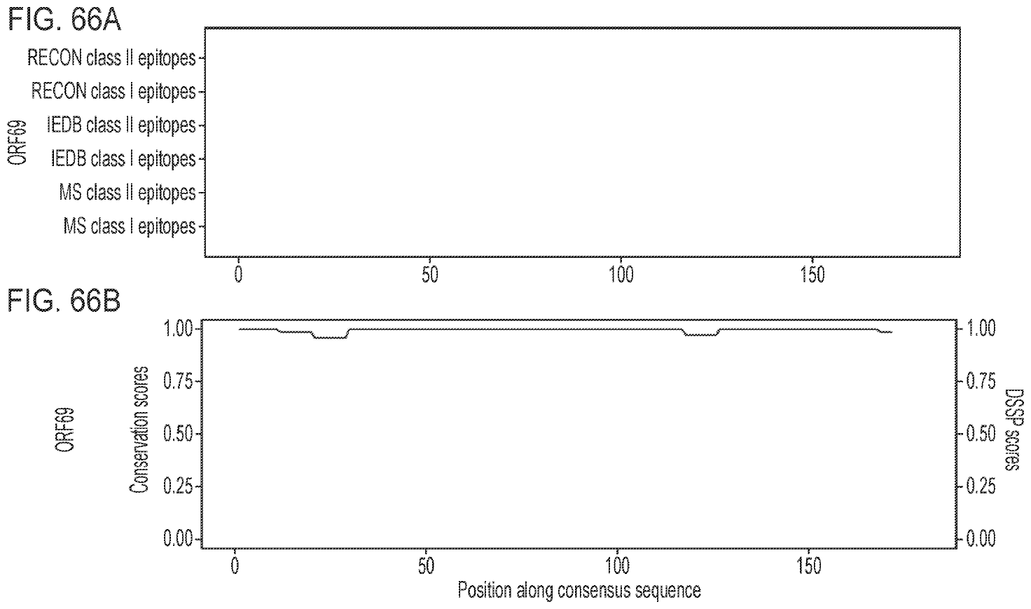

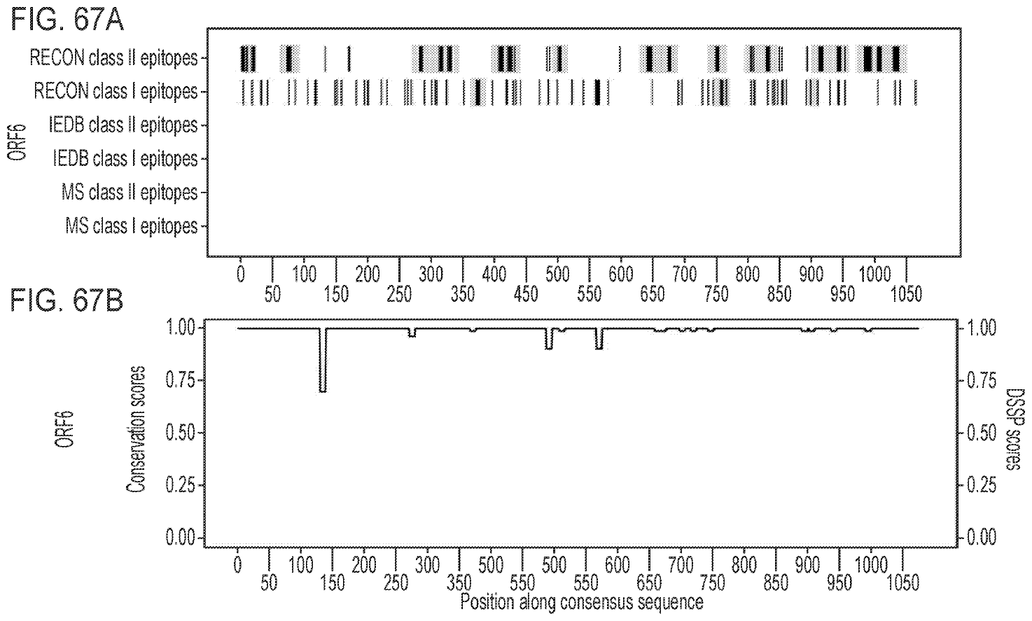

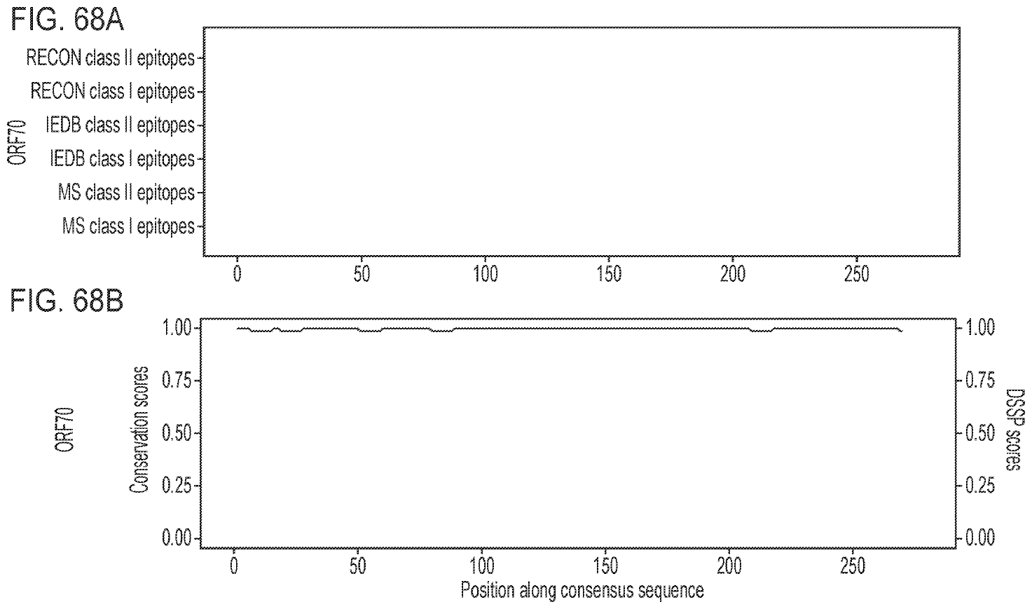

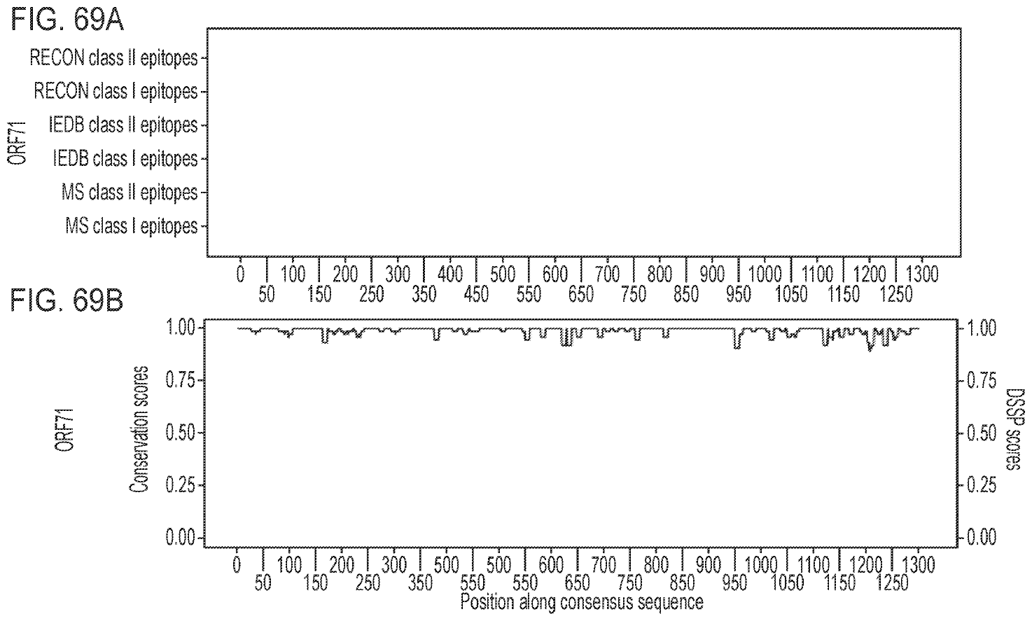

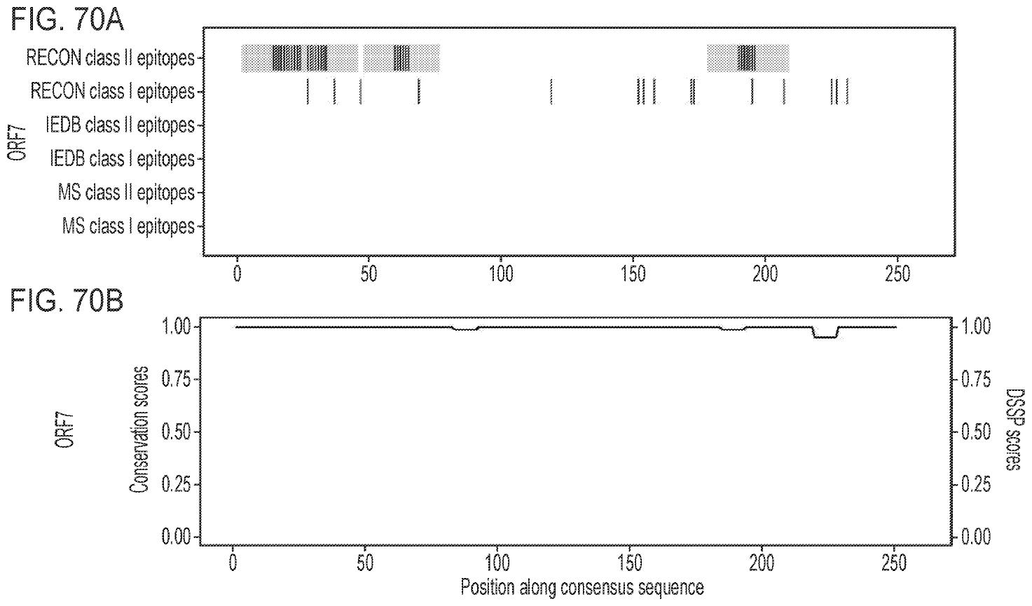

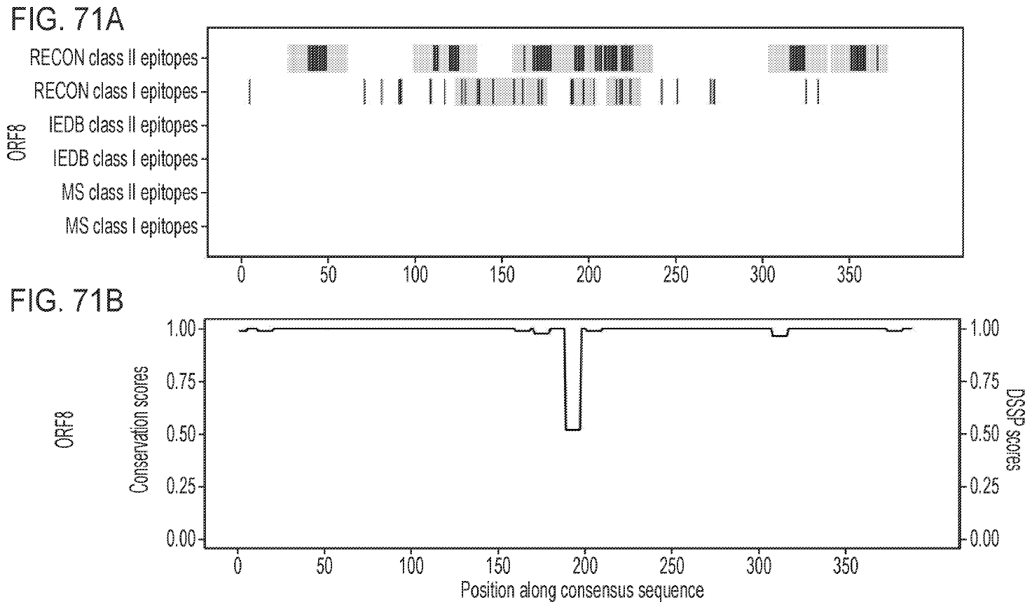

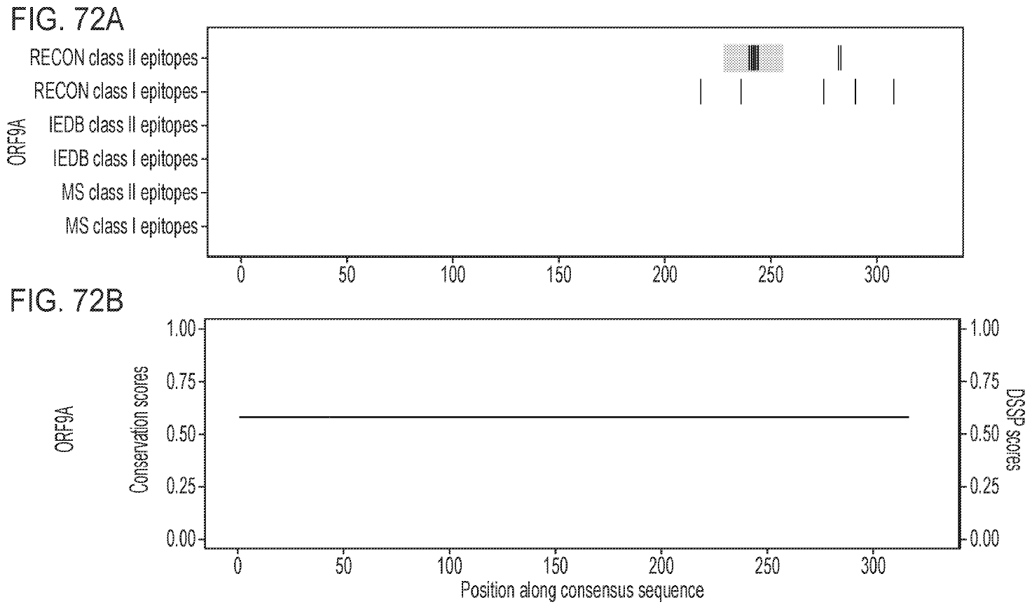

In Supplemental FIG. 4 and Supplementary Table 3, Ouwendijk, et al. provided protein abundance from ARPE-19 cells infected with VZV and grown in SILAC media to differentiate inoculum VZV proteins from newly synthesized viral proteins. Label free quantification was used to estimate protein abundance. See Ouwendijk et al. “Analysis of Virus and Host Proteomes During Productive HSV-1 and VZV Infection in Human Epithelial Cells.” Front Microbiol. 2020; 11:1179. Published 2020 May 29. doi:10.3389/fmicb.2020.01179, the entire contents of which are incorporated herein by reference for the purposes described herein.

Protein abundance for HSV-1 infected microsomal membranes was taken from Table 1 from Saiz-Ros et al. (“Host Vesicle Fusion Protein VAPB Contributes to the Nuclear Egress Stage of Herpes Simplex Virus Type-1 (HSV-1) Replication.” Cells. 2019; 8(2):120. doi:10.3390/cells8020120) and gene homologies were used to associate these abundances with Varicella genes from Cohen “The Varicella Zoster Genome,” Curr Top Microbiol Immunol. 2010; 342:1-14. doi:10.1007/82_2010_10. The entire contents of the foregoing references are incorporated herein by reference for the purposes described herein. Protein abundance was based on label free spectral-counts to estimate the relative levels of the proteins detected by mass spectrometry.

Ligandomics HHV6B data was downloaded from MassIVE protein repository (MSV000083546) and re-searched using Spectrum Mill software package (version 131.07.04.210) against all UCSC Genome Broser genes (January 2018), common contaminants, and HHV-6B viral database (uniprot-proteome_UP000006930.fasta). Searches included oxidated methionine set as a variable modification, and a minimum scored peak intensity of 50% & PSM FDR estimate <1% was used to filter results. Homology between HHV-6 and VZV was determined from Table 1 of Nicholas “Determination and analysis of the complete nucleotide sequence of human herpesvirus.” J Virol. 1996; 70(9):5975-5989. doi:10.1128/JVI.70.9.5975-5989.1996, the entire contents of which are incorporated herein by reference for the purposes described herein.

### IEDB Data Curation Process:

MHC ligands, T-cell epitopes, and B-cell epitopes were collected from IEDB from each virus, for example, including the downloadable datasets mhc_ligand_full.csv, tcell_full_v3.csv, and bcell_fullv3.csv, respectively. Filtering and data cleaning was performed to ensure these data only contained relevant epitopes, e.g. negative assay results and non-human viral strains were omitted. In order to summarize the number of unique epitope sequences in these data in an accurate, non-redundant manner, epitopes that were reported in unbiased studies (listed below) were removed. Lastly, the remaining epitopes were classified into six categories indicating the assay used and relevant MHC class, if applicable. T-cell epitopes that were not specified to be CD4+ or CD8+ specific were categorized as “Ambiguous T-cell epitopes”.

### Select Unbiased Studies Include:

1. Laing et al. “Zoster Vaccination Increases the Breadth of CD4+ T Cells Responsive to Varicella Zoster Virus.” J Infect Dis. 2015; 212(7):1022-1031. doi:10.1093/infdis/jiv164
2. Jing et al. “Extensive CD4 and CD8 T Cell Cross-Reactivity between Alphaherpesviruses.” J Immunol. 2016; 196(5):2205-2218. doi:10.4049/jiimmunol.1502366
3. Becerra-Artiles et al. “Naturally processed HLA-DR3-restricted HIV-6B peptides are recognized broadly with polyfunctional and cytotoxic CD4 T-cell responses.” Eur J Immunol. 2019; 49(8):1167-1185. doi:10.1002/eji.201948126)
4. Chiu et al. “Broadly reactive human CD8 T cells that recognize an epitope conserved between VZV, HSV and EBV.” PLoS Pathog. 2014; 10(3):e1004008. Published 2014 Mar. 27. doi:10.1371/journal.ppat.1004008

The entire contents of the aforementioned documents are incorporated herein by reference for the purposes described herein.

Because Varicella and Herpes Simplex Virus Type 1 and 2 (HSV-1 & HSV-2) have significant homology, the above process was repeated for Herpes Simplex Virus Type 1 and 2 (HSV-1 & HSV-2). The data was curated and summarized in the same manner, except the removal of unbiased epitopes was not performed.

Tables 1A-1I present relevant gene-level data curated and/or re-analyzed from literature publications including the datasets described above. Such data include, for example, gene expression, protein expression, HLA ligandomics, and immunogenicity. The tables also identify genes that can be useful for a vaccine described herein per systematic analysis and ranking process based on the exemplary curated data noted above. In some embodiments, VZV conservation analysis was based on 101 VZV strains.

### Example 2: Exemplary Methods for Assessing Suitability of VZV Genes as Targets for CD8+ and CD4+ T Cell Responses

The present Example provides exemplary methods for identifying VZV genes as targets for CD8+ and/or CD4+ T cell responses. In some embodiments, VZV genes identified as targets for CD8+ and/or CD4+ T cell responses may be useful for immunogenic compositions (e.g., vaccine compositions, and epitope binding agents, etc.). In some embodiments, regions and/or epitopes encoded by VZV genes identified using the methods described herein can be expressed by VZV during a latent phase of its life cycle and/or during an active infection. In some embodiments, such regions and/or epitopes of VZV antigens can be or comprise CD4+ and/or CD8+ T cell epitopes. In some embodiments, such regions and/or epitopes of VZV antigens can be or comprise predominantly CD4+ and/or CD8+ T cell epitopes.

Various lines of evidence were considered to assess the suitability of VZV genes as targets for CD8+ and CD4+ T cell responses. Depending on availability and applicability, RNA expression, protein expression, HLA ligandomics, and/or immunogenicity data from literature publications including the datasets as described in Example 1 were considered. In some embodiments, immunogenicity data was considered to be of higher importance if it was derived from an “unbiased study” (one which assessed genes across the whole viral proteome) than if it was derived from a “biased study” (one which only assessed immunogenicity for a single gene or a subset of genes in the viral proteome). Tables 2A-2B outline a step-wise process by which genes were tiered and/or eliminated from the final roster of selections. Lowest tier number (e.g., “Tier 1”) indicates highest importance. The “Description” column indicates the overall concept of the selection step; whereas the column “Selection Criteria” indicates the specific rule or thresholds used. The column “Genes” lists the genes that correspond to the given priority tier.

### Example 3: Exemplary Methods for VZV Multiple Sequence Alignments

For each protein, strain variants were aligned into a global sequence alignment, for example, using MAFFT (default parameters; https://www.ebi.ac.uk/Tools/msa/mafft/). Cross-strain conservation across each protein sequence was determined, for example, by applying a sliding 9mer window and calculating the frequency of the most frequent 9mer at each position. Furthermore, experimentally defined structures analyzed by the DSSP algorithm (Touw et al. “A series of PDB related databases for everyday needs.” Nucleic Acids Research (2015 January); 43(Database issue): D364-D368; Kabsch and Sander “Dictionary of protein secondary structure: pattern recognition of hydrogen-bonded and geometrical features.” Biopolymers. (1983) 22: 2577-2637. PMID: 6667333; UI: 4128824) were accessed (https://swift.cmbi.umcn.nl/gv/dssp/) and inspected to determine solvent accessibility scores per protein position (likewise using a 9mer rolling average across the protein). Structures were not available for all proteins or all protein regions. The sequence was further annotated by indicating the starting positions of peptides highly scored by T cell epitope prediction tools from the bioinformatics pipeline RECON® (Real-time Epitope Computation for Oncology) (Abelin et al. Immunity (2017) 21:46:315-26; Abelin et al., Immunity (2019) 15; 51:766-779.e17) (percent rank <0.1 for HLA-I; percent rank <0.5 for HLA-II) for alleles that are common in people of European ancestry (A*02:01, A*01:01, A*03:01, A*24:02, A*11:01, B*07:02, B*08:01, B*44:02, B*35:01, B*44:03, C*07:01, C*07:02, C*04:01, C*06:02, C*03:04, DRB1*07:01, DRB1*15:01, DRB1*03:01, DRB1*01:01, DRB1*04:01, DRB4*01:03, DRB3*01:01, DRB3*02:02, DRB5*01:01, DRB3*03:01, DPA1*01:03/DPB1*04:01, DPA1*01:03/DPB1*02:01, DPA1*01:03/DPB1*04:02, DPA1*01:03/DPB1*03:01, DPA1*02:01/DPB1*01:01, DQA1*01:02/DQB1*06:02, DQA1*05:01/DQB1*02:01, DQA1*02:01/DQB1*02:02, DQA1*05:05/DQB1*03:01, DQA1*01:01/DQB1*05:01; these allele selections could be revised to match the allele frequencies applicable in the region(s) of vaccine distribution). Other peptides of interest (those identified by HLA ligandomics studies or otherwise reported in The Immune Epitope Database (iedb.org)) were also positionally located in the global sequence alignments. IEDB peptides were extracted from the tables tcell_full_v3.zip, bcell_full_v3.zip, and mhc_ligand_full.zip as accessed on Jul. 27, 2021. The entire contents of the aforementioned documents are incorporated herein by reference for the purposes described herein.

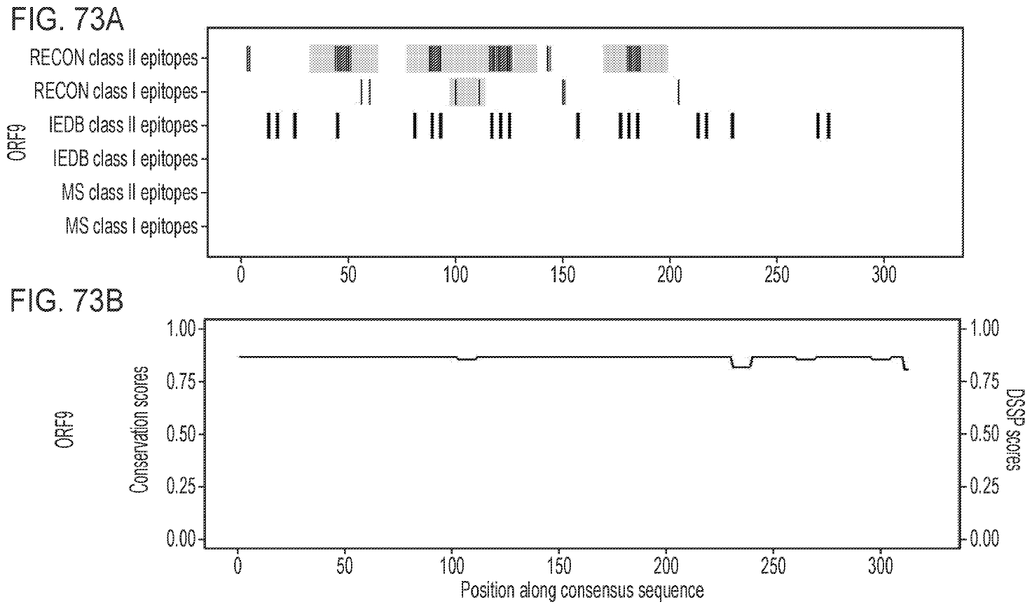

FIGS. 1A-73B shows cross-strain sequence conservation and epitopes of interest for an indicated VZV gene. Conservation is determined according to a sliding 9mer window as described above, where 0 indicates no conservation and 1 indicates that all strains are identical in the given region. Epitopes of interest include those highly predicted by RECON (percent rank <0.1 for HLA-I; percent rank <0.5 for HLA-II; for alleles common among individuals with European ancestry), identified by mass spectrometry-based HLA ligandomics, or otherwise identified in The Immune Epitope Database (iedb.org). It should be noted that positions are indexed with respect to the multiple sequence alignment, which contains gap characters.

### Example 4: Exemplary Methods for Determination of VZV “Block” Regions and String Design

The present Example provides exemplary methods for identifying “block” regions of a VZV polypeptide that may be useful for immunogenic compositions (e.g., vaccine compositions, and epitope binding agents, etc.). In some embodiments, “block” regions of a VZV polypeptide can be expressed by VZV during a latent phase of its life cycle and/or during an active infection. In some embodiments, such “block” regions can be or comprise CD4+ and/or CD8+ T cell epitopes. In some embodiments, such “block” regions can be or comprise B cell epitopes. In some embodiments, such “block” regions can be or comprise predominantly CD4+ and/or CD8+ T cell epitopes.

In some embodiments, one or more “block” regions identified in the present Example can be encoded by a polynucleotide. In some embodiments, such a polynucleotide can be a polyribonucleotide (e.g., in some embodiments mRNA). In some embodiments, a plurality of (including, e.g., at least two, at least three, at least four, at least five, at least six, at least seven, at least eight, at least nine, at least ten, or more) “block regions” identified in the present Example can be linked by one or more linkers. In some embodiments, such a polynucleotide can comprise a secretory signal sequence (e.g., in some embodiments at the N-terminal of the polynucleotide). In some embodiments, such a polynucleotide can comprise an MHC trafficking signal sequence (e.g., a MHC class I trafficking signal sequence; MITD sequence), for example, in some embodiments at the C-terminal of the polynucleotide.

For each VZV protein, a sliding window of 15 amino acid positions are applied from start to end over the multiple sequence alignment (MSA) profile over all the sequences belonging to a particular strain. The number of peptides highly scored by RECON (percent rank <0.1 for HLA-I; percent rank <0.5 for HLA-II) for alleles that are common in people of European ancestry (A*02:01, A*01:01, A*03:01, A*24:02, A*11:01, B*07:02, B*08:01, B*44:02, B*35:01, B*44:03, C*07:01, C*07:02, C*04:01, C*06:02, C*03:04, DRB1*07:01, DRB1*15:01, DRB1*03:01, DRB1*01:01, DRB1*04:01, DRB4*01:03, DRB3*01:01, DRB3*02:02, DRB5*01:01, DRB3*03:01, DPA1*01:03/DPB1*04:01, DPA1*01:03/DPB1*02:01, DPA1*01:03/DPB1*04:02, DPA1*01:03/DPB1*03:01, DPA1*02:01/DPB1*01:01, DQA1*01:02/DQB1*06:02, DQA1*05:01/DQB1*02:01, DQA1*02:01/DQB1*02:02, DQA1*05:05/DQB1*03:01, DQA1*01:01/DQB1*05:01; these allele selections could be revised to match the allele frequencies applicable in the region(s) of vaccine distribution) that map to each of these windows are counted. Sliding windows with at least 5 such peptides are considered as “dense epitope regions.” Dense epitope regions are determined for HLA-I and HLA-II peptides separately, and for each type of peptides, dense epitope regions are merged into continuous blocks. These continuous blocks are then filtered to only contain intervals where the corresponding protein conservation scores previously determined are higher than 0.9.

For each block identified from the above steps, the reference sequence for the corresponding virus is then used to determine sequence—sequences encompassing the block interval plus at least 10 amino acids (including, e.g., at least 11 amino acids, at least 12 amino acids, at least 13 amino acids, at least 14 amino acids, at least 15 amino acids, or more) downstream and upstream of the block interval are taken to form chunk sequences. In some embodiments, sequences encompassing the block interval plus 14 amino acids downstream and 15 amino acids upstream of the block interval are taken to form chunk sequences. The chunk sequences are filtered to contain only prioritized genes, and linked together using linkers (e.g., but not limited to linkers described herein such as, e.g., GSS linkers) with a secretary signal peptide domain (SP domain) in the N terminal and MITD domain in the C terminal. Exemplary sequences for the linker, N-terminal domain, and C-terminal domain are provided as follows:
SP domain: MKMRVMAPRTLILLLSGALALTETWAGS
MITD domain: IVGIVAGLAVLAVVVIGAVVATVMCRRKSSGGKGGSYSQAA SSDSAQGSDVSLTA
Start GSS linker (after SP domain): GGSGGGGSGG
Middle GSS linker (between blocks): GGSGGGGSGG
End GSS linker (before MITD): GGSLGGGGSG

The corresponding nucleotide sequence can be determined by reverse translation using the wild-type viral sequence, codon optimization, and/or some other codon selection scheme that optimizes one or more aspects of the resulting RNA string (e.g., stability, the lack of hairpins, etc.).

Tables 3A-3B contain CD8 and CD4-focused blocks, respectively, identified from the analyses as described above, as well as the corresponding string design. The columns “start” and “end” denote the starting and ending position of each identified region in the corresponding reference strain (Dumas for Varicella-zoster virus). The “gene” column provides the official gene symbol for each region, and the “seq” column provides the reference/exemplary strain sequence for that region. The final row of each table, with gene name “full_string” gives the GSS linker-linked exemplary string design, with SP domain in the N terminal and MITD domain in the C terminal.

### Example 5: Exemplary Methods for Determination of HLA Binding Peptides (VZV)

All strain variants were downloaded from https://www.viprbrc.org/. All pairwise strain homologies were assessed by comparing the overlap of 9mer substrings of the constituent proteins; for a pair of strains, the number of 9mers present in both strains (the intersection) was divided by the number of 9mers present in at least one strain (the union) to derived a conservation score between 0 (no homology) and 1 (perfect homology). Outlier strains with poor homology to most other strains were excluded as a data cleaning procedure. The strain “Dumas” was chosen as an exemplar strain (NCBI Reference Sequence: NC 001348.1) for VZV and used for sequence indexing unless otherwise noted. The protein sequences of the remaining strains were dissolved into all possible substrings (“candidate HLA binding peptides”) of lengths 8-12. Candidate HLA binding peptides of lengths 8-12 were scored for binding potential to 105 HLA-I alleles using an algorithm called neonmhc1. Candidate HLA binding peptides of lengths 12-20 were scored for binding potential to 85 HLA-II alleles using an algorithm called neonmhc2. Neonmhc1 is a proprietary algorithm based on proprietary data using methods similar to those published in Abelin et al. “Mass spectrometry profiling of HLA-associated peptidomes in mono-allelic cells enables more accurate epitope prediction” Immunity (2017) 46(2):315-326, the entire contents of which are incorporated herein by reference for purposes described herein. Neonmhc2 is a proprietary algorithm based on proprietary data using methods similar to those published in Abelin et al., “Defining HLA-II ligand processing and binding rules with mass spectrometry enhances cancer epitope prediction” Immunity (2019) 51(4):766-779.e17, the entire contents of which are incorporated herein by reference for purposes described herein. These two algorithms are part of a larger suite of proprietary analytical tools referred to as “RECON”; neonmhc1 predictions and neonmhc2 predictions are both occasionally referred to as “RECON” predictions herein. Neonmhc1 and neonmhc2 predictions are made on a continuous “percent rank” scale (Nielsen and Andreatta, “NetMHCpan-3.0; improved prediction of binding to MHC class I molecules integrating information from multiple receptor and peptide length datasets” Genome Medicine (2016) 8:33, the entire contents of which are incorporated herein by reference for purposes described herein) where smaller values indicate a higher likelihood of binding. Conservation was determined for each peptide by calculating the percentage of strains with at least one instance of the peptide within their proteome. Thus, a peptide with a conservation of 60% appears is encoded by 60% of the viral strains.

Table 5A contains peptides grouped according to likelihood of HLA-binding (per allele) and conservation predicted by neonmhc1, wherein sequences of such peptides have been submitted via DVD and are incorporated herein by reference in their entirety. “Very strong” indicates a percent rank binding score less than 0.1%. “Strong” indicates a percent rank binding score between 0.1% and 1%. “Moderate” indicates a percent rank binding score between 1% and 2%. Peptides with weaker predicted binding scores are not included in the table. “High conservation” indicates that the peptide is present in >90% of strains. “Moderate conservation” indicates that the peptide is present in 50%-90% of strains. “Poor conservation” indicates that the peptide is present in <50% of strains.

Table 5B contains peptides grouped according to likelihood of HLA-binding (per allele) and conservation predicted by neonmhc2, wherein sequences of such peptides have been submitted via DVD and are incorporated herein by reference in their entirety. “Very strong” indicates a percent rank binding score less than 0.5%. “Strong” indicates a percent rank binding score between 0.5% and 2%. “Moderate” indicates a percent rank binding score between 2% and 5%. Peptides with weaker predicted binding scores are not included in the table. “High conservation” indicates that the peptide is present in >90% of strains. “Moderate conservation” indicates that the peptide is present in 50%-90% of strains. “Poor conservation” indicates that the peptide is present in <50% of strains.

HLA binding peptides identified herein (e.g., HLA-I binding peptides and/or HLA-II binding peptides) can be delivered by immunogenic compositions described herein. In some embodiments, one or more HLA binding peptides (e.g., HLA-I binding peptides and/or HLA-II binding peptides) selected from Tables 5A-5B herein can be encoded by one or more polynucleotides (e.g., RNA) described herein. In some embodiments, a polynucleotide (e.g., RNA) described herein can encode a polyepitopic polypeptide comprising a plurality of (including, e.g., at least 2, at least 3, at least 4, at least 5, at least 6, at least 7, at least 8, at least 9, at least 10, at least 15, at least 20, at least 25, at least 30, or more) HLA binding peptides (e.g., HLA-I binding peptides and/or HLA-II binding peptides) selected from Tables 5A-5B herein.

### Example 6: Exemplary Methods for Determination of T Cell Epitopes (VZV)

In order to prioritize CD4 and CD8 epitopes for RNA string design for each virus, in some embodiments all known immunogenic and/or HLA-presented epitopes were first considered. Known immunogenic epitopes were curated from IEDB, only considering relevant T-cell epitopes, for example, in some embodiments only CD4 T-cell epitopes were considered for the CD4 shortlist. In some embodiments, B-cell epitopes and MHC ligands from IEDB were omitted. Known HLA-presented epitopes were curated from the HLA ligandomics datasets re-searched via mass spectroscopy (e.g., SpectraMill). Depending on the number of immunogenic and/or HLA-presented epitopes for the given virus and T-cell subset (CD4 or CD8), one of two procedures as described below were followed:
1) If the number of epitopes exceeded the maximum amount of epitopes desired (e.g., 70 for CD4, 100 for CD8), then epitopes were filtered out based on the following criteria, applied procedurally until less than the maximum amount of epitopes desired remained:
   Remove epitopes poorly conserved in viral strains (<50%) & with poor RECON prediction values for any prioritized HLA allele (>2%-rank for CD8, >5%-rank for CD4). A list of prioritized HLA alleles is provided below.
   Remove epitopes moderately conserved in viral strains (<90%).
   Remove epitopes with moderate RECON predictions for any prioritized HLA allele (>1%-rank for CD8, >2%-rank for CD4).
   Remove epitopes with strong RECON predictions for any prioritized HLA allele (>0.1%-rank for CD8, >0.5%-rank for CD4).
2) If the number of epitopes was less than the minimum amount of epitopes desired (e.g., 40 for CD4, 60 for CD8), then epitopes were supplemented using the following logic: for each prioritized HLA allele (e.g., as described below), epitopes were selected based on the strength of RECON predictions. Only epitopes which were highly conserved in viral strains (>90%) were considered. Epitopes were selected until the maximum number of epitopes desired would be exceeded while keeping the number of epitopes selected per allele equal.

HLA allele prioritization: The top 5 most frequent HLA alleles by US population (data from Gragert et al., “Six-locus high resolution HLA haplotype frequencies derived from mixed-resolution DNA typing for the entire US donor registry” Human Immunology (2013) 74(10): 1313-1320, the entire contents of which are incorporated herein by reference for the purposes described herein) were selected for this analysis. For Class I (CD8), this included the A, B, and C loci. For Class II (CD4), this included the DRB1, DRB345, DQ, and DP loci. If desirable, additional HLA alleles can be included for analysis.

The shortlisted epitopes were designed as RNA strings using approaches described below for HLA-I epitopes and HLA-II epitopes. For HLA-I epitopes, all pairwise epitope orderings were assessed for their likelihood of producing proteasomal cleavage events at the desired position. This was done using a proprietary cleavage predictor trained using proprietary data using methods similar to those presented in Abelin et al. “Mass spectrometry profiling of HLA-associated peptidomes in mono-allelic cells enables more accurate epitope prediction” Immunity (2017) 46(2): 315-326, the entire contents of which are incorporated herein by reference for the purposes described herein. Treating these pairwise scores as a directed graph wherein each epitope represents a node and each cleavage score represents an edge-wait, a heuristic approach was used (see, e.g., Hahsler M, “TSP: Traveling Salesperson Problem (TSP). R package version 1.1-10 (2020), which can be online accessible at https://CRAN.R-project.org/package=TSP; Hahsler M, “TSP-Infrastructure for the traveling salesperson problem” Journal of Statistical Software (2007) 23(2): 1-21, the entire contents each of which are incorporated herein by reference for the purposes described herein) to traverse the graph while maximizing on-target cleavability (noting that while the solution was referred as “optimal”, the algorithm is only a heuristic and not guaranteed to provide the globally optimal solution). Additionally or alternatively, in some embodiments, additional amino-acid linker sequences can be added between epitope pairs as needed to redress any adjacencies that that fail to meet suitable cleavage prediction scores. For HLA-II epitopes, the sequences were concatenated without linkers or special considerations for ordering. However, both approaches for HLA-I epitopes and HLA-II epitopes can be used to meet design objectives (such as reducing the presence of non-target junctional epitopes or achieving desired biophysical properties in the resultant polypeptide).

The top portion of Table 4A contains HLA-I epitopes (e.g., CD8 epitopes) shortlisted for inclusion in a string-of-epitopes vaccine, while the remaining portion of Table 4A contains HLA-II epitopes (e.g., CD4 epitopes) shortlisted for inclusion in a string-of-epitopes vaccine. Epitopes represent a mix of informatic predictions (per RECON) and sequences that have been defined as presented or immunogenic experimentally (per literature).

Table 4B in part shows the amino acid sequence corresponding to a candidate RNA string encoding a sequence of prioritized HLA-I epitopes. In some embodiments, the epitope sequences are ordered in a manner that maximizes the likelihood that the target epitopes will be processed and presented (see Methods as described above). In some embodiments, the sequence can additionally or alternatively be designed to minimize non-target junctional epitopes (for example, by choosing an epitope ordering that results epitope adjacencies unlikely to produce peptides that bind common HLAs), to achieve desired conformational properties in the resultant polypeptide (e.g., an enrichment of alpha helices, which could likewise be achieved by selecting an ordering with optimal epitope adjacencies), or some combination of the three objectives described herein. In some embodiments, one or more linker sequences can be additionally or alternatively used to achieve the above-described objectives. In some embodiments, the corresponding nucleotide sequence can be determined by reverse translation using the wildtype viral sequence, codon optimization, and/or some other codon selection scheme that optimizes one or more aspects of the resulting RNA string (e.g., but not limited to stability, the lack of hairpins, etc.).

Table 4B also shows the amino acid sequence corresponding to a candidate RNA string encoding a sequence of prioritized HLA-II epitopes. In some embodiments, by permuting the epitopes and/or adding one or more linkers, the sequence could be further optimized to achieve specific features (e.g., lack of non-target junctional epitopes and/or presence of desired polypeptide properties, as described herein). In some embodiments, the corresponding nucleotide sequence can be determined by reverse translation using the wildtype viral sequence, codon optimization, and/or some other codon selection scheme that optimizes one or more aspects of the resulting RNA string (e.g., but not limited to stability, the lack of hairpins, etc.).

### Example 7: Exemplary RNA Constructs Encoding VZV Antigens

The present example describes certain exemplary VZV antigens, and sequences encoding them, that may be utilized in certain embodiments of the present disclosure.

In some embodiments, an antigen delivered in accordance with the present disclosure is or comprises a VZV protein or an immunogenic portion thereof (e.g., comprising one or more epitopes thereof) as indicated in Table 1A or a VZV protein encoded by a gene as listed in Tables 2A-2B or an immunogenic portion thereof (e.g., comprising one or more epitopes thereof). In some embodiments, an antigen delivered in accordance with the present disclosure is or comprises one or more regions of one or more VZV proteins, which regions each independently comprise one or more CD4+ epitopes and/or one or more CD8+ epitopes as indicated in Tables 3A-3B. In some embodiments, an antigen delivered in accordance with the present disclosure is or comprises a VZV peptide as indicated in Table 4A, Table 5A, or Table 5B. In some embodiments, such an antigen is delivered by administration of an RNA encoding such an antigen.

In some particular embodiments, an administered RNA has a structure:
Structure 1: m₂^{7,3′-O}Gppp(m₁^{2′-O})ApG-hAg-Kozak-SEC-Immunogen —FI-A30L70, wherein m₂^{7,3′-O}Gppp(m₁^{2′-O})ApG=5′ cap; hAg=5′ UTR human alpha-globin; SEC=secretory signal or signal peptide (SP); Immunogen=a nucleotide sequence comprising a sequence that encodes an antigen described herein; FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence.

In some embodiments, an administered RNA has a structure:
Structure 2: m₂^{7,3′—O} Gppp(m₁^{2′-O})ApG-hAg-Kozak-SEC-Immunogen-MITD-FI-A30L70, wherein m₂^{7,3′-O}Gppp(m₁^{2′-O})ApG=5′ cap; hAg=5′ UTR human alpha-globin; SEC=signal peptide (SP); Immunogen=a nucleotide sequence comprising a sequence that encodes one or more antigens described herein; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence.

In some embodiments, an Immunogen sequence encodes a plurality of immunogenic fragments (e.g., epitopes) from an antigen. In some embodiments, an Immunogen sequence encodes a plurality of immunogenic fragment (e.g., epitopes) from two or more antigens. In some embodiments. In some embodiments, such immunogenic fragments are linked together to form an Immunogen sequence by linkers (e.g., in some embodiments a linker that is enriched in G and/or S amino acid residues). In some embodiments, a linker may be or comprise an amino acid sequence of GGSGGGSGG. In some embodiments, a linker may be or comprise an amino acid sequence of GGSLGGGGSG.

### Example 8: Exemplary RNA Constructs Encoding Multiepitope VZV Antigens

The present example describes certain exemplary VZV multiepitope antigens, and sequences encoding them, that may be utilized in certain embodiments of the present disclosure.

### A) Exemplary Construct Encoding a VZV Multi-Epitope Polypeptide #1

Structure: m₂^{7,3′-O}Gppp(m₁^{2′-O})ApG-hAg-Kozak-SEC-CD8 string-MITD-FI-A30L70, wherein m₂^{7,3′-O}Gppp(m₁^{2′-O})ApG=5′ cap; hAg=5′ UTR human alpha-globin; SEC=signal peptide (SP); CD8 string=a nucleotide sequence comprising a sequence that encodes a plurality of (e.g., two or more) CD8+ T cell epitopes described herein; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence. In some embodiments, the MITD element may be absent.

In some embodiments, a CD8 string may comprise at least 2 (including, e.g., at least 3, at least 4, at least 5, at least 10, at least 15, at least 20, or more) CD8+ T cell epitopes and/or HLA-I epitopes as listed in Table 4A or Table 5A. In some embodiments, a CD8 string may comprise an amino acid sequence as recited in Table 4B.

### B) Exemplary Construct Encoding a VZV Multi-Epitope Polypeptide #2

Structure m₂^{7,3′-O}Gppp(m₁^{2′-O})ApG-hAg-Kozak-SEC-CD4 string-MITD-FI-A30L70, wherein m₂^{7,3′-O}Gppp(m₁^{2′-O})ApG=5′ cap; hAg=5′ UTR human alpha-globin; SEC=signal peptide (SP); CD4 string=a nucleotide sequence comprising a sequence that encodes a plurality of (e.g., two or more) CD4+ T cell epitopes described herein; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence. In some embodiments, the MITD element may be absent.

In some embodiments, a CD4 string may comprise at least 2 (including, e.g., at least 3, at least 4, at least 5, at least 10, at least 15, at least 20, or more) CD4+ T cell epitopes and/or HLA-II epitopes as listed in Table 4A or Table 5B. In some embodiments, a CD4 string may comprise an amino acid sequence as recited in Table 4B.

### C) Exemplary Construct Encoding a VZV Multi-Epitope Polypeptide #3

Structure m₂^{7,3′-O}Gppp(m₁^{2′-O})ApG-hAg-Kozak-SEC-CD8 chunk sequence-MITD-FI-A30L70, wherein m₂^{7,3′O}Gppp(m₁^{2′-O})ApG=5′ cap; hAg=5′ UTR human alpha-globin; SEC=signal peptide (SP); CD8 chunk sequence=a nucleotide sequence comprising a sequence that encodes one or more regions of one or more VZV polypeptides, wherein each region predominantly comprises CD8+ epitopes; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence. In some embodiments, the MITD element may be absent.

In some embodiments, a CD8 chunk sequence may comprise at least 2 (including, e.g., at least 1, at least 2, at least 3, at least 4, at least 5, at least 10, at least 15, at least 20, or more) immunogenic fragment(s) of polypeptide(s) encoded by gene(s) or peptide(s) as listed in Table 3A. In some embodiments, a sequence comprising SEC, CD8 chunk sequence, and MITD is represented by the sequence denoted as “full string” in Table 3A.

### D) Exemplary Construct Encoding a VZV Multi-Epitope Polypeptide #4

Structure m₂^{7,3′-O}Gppp(m^{2′-O})ApG-hAg-Kozak-SEC-CD4 chunk sequence-MITD-FI-A30L70, wherein m₂^{7,3′-O}Gppp(m₁^{2′-O})ApG=5′ cap; hAg=5′ UTR human alpha-globin; SEC=signal peptide (SP); CD4 chunk sequence=a nucleotide sequence comprising a sequence that encodes one or more regions of one or more VZV polypeptides, wherein each region predominantly comprises CD4+ epitopes; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence. In some embodiments, the MITD element may be absent.

In some embodiments, a CD4 chunk sequence may comprise at least 2 (including, e.g., at least 1, at least 2, at least 3, at least 4, at least 5, at least 10, at least 15, at least 20, or more) immunogenic fragment(s) of polypeptide(s) encoded by gene(s) or peptide(s) as listed in Table 3B. In some embodiments, a sequence comprising SEC, CD4 chunk sequence, and MITD is represented by the sequence denoted as “full string” in Table 3B.

### Example 9: Exemplary VZV Peptide String Designs

The present Example exemplifies certain constructs (referred to herein as “strings”) of multiple VZV epitopes linked to one another and useful, for example, in immunogenic compositions (e.g., vaccine compositions) or otherwise as described herein.

Strings described in the present Example are designed to contain specific epitopes of VZV, each of which is disclosed herein and, e.g., is predicted and/or selected as described here, for example through use of an MHC-binding algorithm as described herein. The strings presented in the present Example are designed for therapeutic use in preventing and/or treating VZV infection (and in certain embodiments VZV latent infection) and can be administered as polynucleotide constructs, e.g., mRNA encapsulated in a lipid nanoparticle.

In some embodiments, strings exemplified herein are encoded in an RNA that includes a 5′-UTR and 3′-UTR. Epitopes are interconnected by peptide linkers, encoded by their respective polynucleotide sequences. In some embodiments, one or more linkers may have a specific cleavage site.

Exemplary amino acid sequences for exemplary strings denoted as “full_string” in Tables 3A-3B, each of which includes a SP domain, GS-enriched linkers, and a MITD domain. Table 4B shows additional exemplary amino acid sequences for exemplary CD8 string and CD4 strings. In some embodiments, polynucleotide sequences are codon optimized (e.g., for efficient translation in humans).

### Example 10: Exemplary VZV Antigen Identification, Selection and/or Characterization

The present Example describes identification, selection and/or characterization of certain VZV protein sequences useful as or in (i.e., as part of) antigens as described herein.

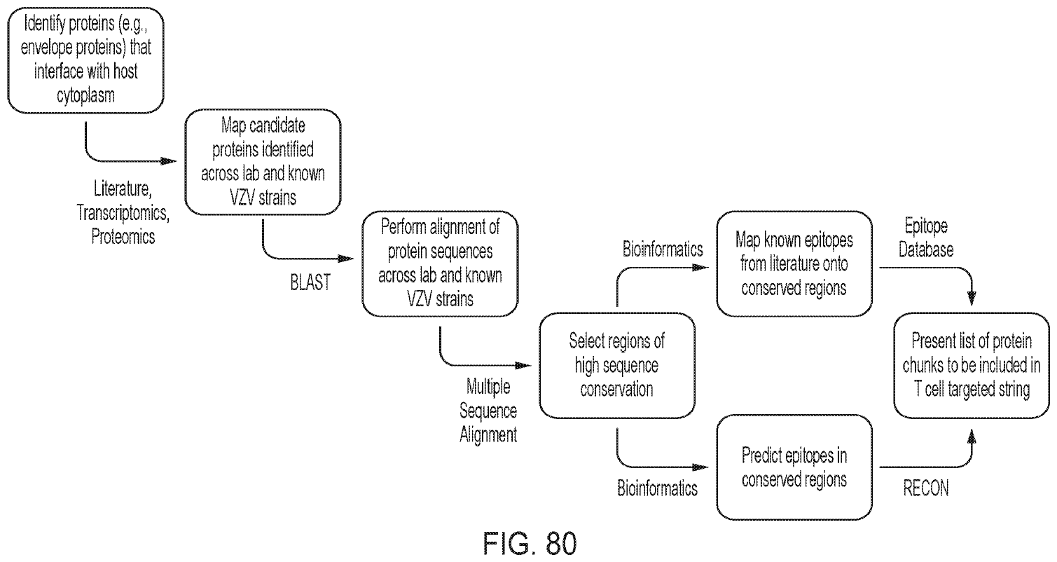

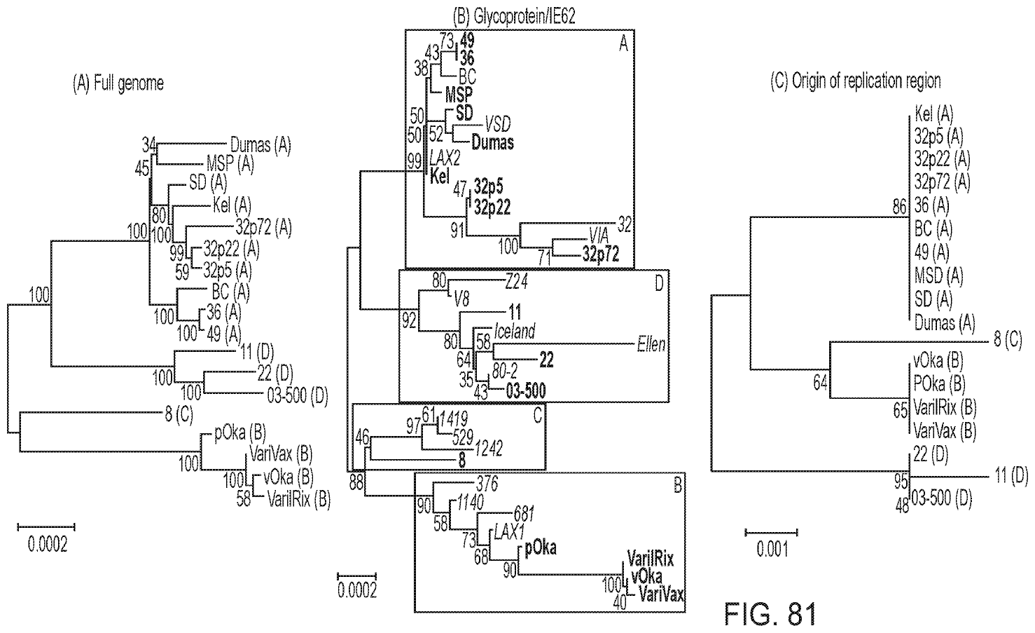

FIG. 80 presents a flow diagram of a process used to identify, characterize, and/or select certain VZV protein sequences (e.g., particular variants) and/or fragments or epitopes thereof, that may be particularly useful in the practice of the present invention. In some embodiments, a utilized antigen is or comprises a VZV protein, or fragment or epitope thereof, as identified or characterized as described in the present Example.

As depicted, proteins expressed prior to cell infiltration/infection and include one or more portions expected or known to interface with host cytoplasm are identified, for example by literature review, considering transcriptomic (i.e., RNA expression levels) and/or proteomic (i.e., expressed protein levels) data. Degree of conservation of candidate proteins across relevant VZV strains (e.g., in relevant geographic region) is considered.

Various lab and field isolate strains can be considered for assessing conserved proteins and T cell epitopes. In some embodiments, strain Dumas was considered for assessing conserved proteins and T epitopes.

Immunogenicity of conserved proteins was also considered, for example by review of literature and/or application of predictive algorithms as described herein.

In accordance with the present example, in some embodiments, each of the VZV proteins encoded by genes shown in Tables 2A-2B and combinations thereof were identified as of particular interest. In some embodiments, one or more of these proteins, or one or more fragments or epitopes thereof is used as or included in an antigen. In some embodiments, a peptide or protein is encoded by an ORF18 gene. In some embodiments, a peptide or protein is encoded by a gene selected from: ORF50, ORF37, ORF48, ORF41, ORF12, ORF18, ORF27, ORF38, ORF19, ORF24, ORF59, ORF62, ORF68, ORF9, ORF67, ORF36, ORF63, ORF29, and combinations thereof.

In some embodiments, an antigen may be or comprise one or more, and specifically may comprise a plurality, of distinct portions (e.g., epitope-containing fragments) of one or more of these proteins, for example in a string construct as described herein. In some embodiments, exemplary CD8 string candidates were designed utilizing one or more epitope-containing fragments of a plurality of proteins encoded by the following genes: ORF10, ORF18, ORF29, ORF31, ORF34, ORF53, and ORF67; representative of such epitope-containing fragments are shown in Table 3A, and a corresponding exemplary string candidate is shown in Table 3A (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA)

In some embodiments, exemplary CD4 string candidates were designed utilizing one or more epitope-containing fragments of a plurality of proteins encoded by the following genes: ORF23, ORF28, ORF36, ORF37, ORF38, ORF40, and ORF44; representative of such epitope-containing fragments are shown in Table 3B, and a corresponding exemplary string candidate is shown in Table 3B (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA)

In some embodiments, a secretory signal (“See”) or a signal peptide (SP) domain present in exemplary string candidates described herein (e.g., a CD8 string and/or a CD4 string as described herein) may be that from HSV-2 gD SP MGRLTSGVGTAALLVVAVGLRVVCA; in alternative embodiments, a different secretory signal, e.g., from HSV-1 gD SP, is used. In some embodiments, a signal peptide may be or comprise an IgE signal peptide. In some embodiments, a signal peptide may be or comprise an IgE HC (Ig heavy chain epsilon-1) signal peptide. In some embodiments, a signal peptide that may be useful in accordance with the present disclosure may comprise one of the following sequences:
MDSKGSSQKGSRLLLLLVVSNLLLPQGVVG;
MDWTWILFLVAAATRVHS;
METPAQLLFLLLLWLPDTTG;
MLGSNSGQRVVFTILLLLVAPAYS (Japanese encephalitis PRM signal sequence);
MKCLLYLAFLFIGVNCA (VSVg protein signal sequence);
MWLVSLAIVTACAGA (Japanese encephalitis JEV signal sequence); or
MFVFLVLLPLVSSQC.

In some embodiments, certain chunk boundary considerations are incorporated into the string constructs, for example establishing chunk boundaries to minimize presence of sequences (e.g., epitopes) that may overlap with the human proteome.

### Example 11: Exemplary Vaccine Composition Delivering at Least One VZV Antigen

The present Example describes certain exemplary vaccine compositions.

In some embodiments, a provided vaccine candidate will contain at least 2 RNAs, at least one of which encodes a VZV antigen described herein (e.g., a full length VZV protein or one or more fragments or epitopes thereof, such as a string construct described herein), and optionally at least one of which encodes at least one other conserved VZV protein (or fragment(s) or epitope(s) thereof, such as in a string construct as described herein; in some embodiments such a string construct may include fragments or epitopes from two or more different VZV proteins).

In some embodiments, two or more (e.g., 3, for example 2 of which are/encode VZV antigen string constructs and one of which is/encodes a string construct of a plurality of CD8 and/or CD4 epitopes from other conserved VZV proteins) are formulated together in a single LNP formulation; in other embodiments, individual RNAs may be separately formulated in (the same or different) LNP formulations and such may be mixed together (e.g., in a 1:1 ratio of each RNA, or alternatively in a 1:1 ratio of VZV-antigen-encoding-RNA to “other” RNA so that, for example, for a composition comprising 2 VZV-antigen-encoding RNAs and one other RNA, the ratios would be 0.5:0.5:1).

### Example 12: Exemplary Pre-Clinical Assessment for VZV RNA Vaccine Compositions

The present Example describes certain pre-clinical assessments that may be performed of certain RNA vaccine compositions described herein for treatment and/or prevention of VZV infection and in certain particular embodiments for treatment and/or prevention of VZV latent infection.

In some embodiments, one vaccine candidate is assessed. In some embodiments, more than one different vaccine candidate may be assessed. In some such embodiments, different candidates may vary, for example, in:
RNA platform (e.g., unmodified RNA, modified RNA, saRNA);
Encoded antigen(s);
Number of RNAs;
Non-coding elements of RNA construct (e.g., cap and/or cap-adjacent sequences, 5′-UTR, 3′-UTR, and/or PolyA tail); and/or
Lipid composition of LNP.

In some embodiments, pre-clinical assessment of certain RNA vaccine compositions (e.g., LNP formulated mRNA-based VZV vaccines) comprises one or more of assessment in challenge experiments, assessment of level of protection, assessment of immunogenicity, and/or assessment of functional antibody responses.

LNP formulated mRNA-based VZV vaccines are tested in a challenge model. Non-human primate models, such as Rhesus macaques and Cynomolgus monkey, and/or rodent models, such as C57/B16 mice, Balb/c mice or NODscidIL2Rγnull mice; and/or guinea pig models, inoculated with VZV, are administered a first vaccination and can be administered an additional vaccination (e.g., 1, 2, 3, 4, 5, 6, 7, 8, 9, or 10 additional vaccinations) following the first vaccination. Wherein more than one vaccination is administered, the vaccinations are administered at an interval of 1, 2, 3, 4, 5, 6, 7, or 8 week intervals). Following vaccination, animals are challenged by VZV. Alternatively or additionally, animals are challenged by intravenous, subcutaneous, and/or intramuscular injection of virus-infected lymphocytes. Lymphocytes can be infected with any suitable strain of VZV. Animals are then evaluated for reduced infection of neurons. In some embodiments, a non-human primate and/or mouse is challenged in a plurality of instances (e.g., before first vaccination and/or wherein additional vaccinations are administered, at any time point between or after vaccinations). Following challenge, animals subjected to the study may be assessed according to any method known in the art, including, for example, serology assessment, immunogenicity, level of protection, etc.

In some embodiments, serum antibody characterization and/or serum transfer experiments (e.g., from one vaccinated species to a different non-vaccinated species, e.g., from vaccinated non-human primate to non-vaccinated mouse) are conducted (e.g., to assess protective antibody response).

In some embodiments, certain RNA vaccine compositions of the present disclosure are assessed for level of protection. Level of protection can be assessed according to any suitable method known in the art.

In some embodiments, certain RNA vaccine compositions of the present disclosure are assessed for immunogenicity. For example, ELISA can be used to determine IgG specific (and subclasses thereof) titers and/or avidity of antibodies generated in response to certain RNA vaccine compositions of the present disclosure to VZV antigens. In some embodiments, serum antibody titers against VZV glycoprotein (e.g., gE glycoprotein, etc.) is determined by ELISA using standard methods. In some embodiments, for example, ELISpot (e.g., for CD8+, CD4+ T cells and/or IFNγ) and assessment of pro-inflammatory cytokine responses with splenocytes from immunized and/or challenged animal models and peptide pools derived from vaccine targets can also be assessed. In some embodiments, for example, phenotyping of immune responses (e.g., by flow cytometry) are assessed. In some embodiments, for example, T cell depletion and/or protection assays are conducted to assess immunogenicity (e.g., according to any suitable known method in the art).

In some embodiments, one or more functional responses of antibodies generated in response to certain RNA vaccine compositions of the present disclosure are assessed. Functional antibody responses can be assessed, for example, using a VZV neutralization assay. In some embodiments, a VZV in vitro neutralization assay is performed to evaluate anti-VZV glycoprotein (e.g., gE) antibodies in neutralizing VZV. For example, anti-VZV glycoprotein antibodies are obtained by collecting the sera of animals (e.g., mice) vaccinated with VZV mRNA vaccines. VZV virus are added to the diluted sera and neutralization is allowed to continue for 1 hour at room temperature. ARPE-19 cells are seeded in 96-wells one day before and the virus/serum mixtures are added to ARPE-19 cells at 50-100 pfu per well. The ARPE-19 cells are fixed on the next day and VZV-specific staining is performed. The plates are scanned and analyzed. A neutralization titer is expressed as the highest serum dilution required to achieve a 50% reduction in the number of plaques.

In some embodiments, functional antibody responses can be assessed, for example, using passive transfer studies of sera from immunized animals to naïve animals that are challenged and assessing level of protection.

### Example 13: Exemplary Characterization Studies of VZV Vaccine Candidates or Vaccine Compositions

The present Example describes certain potential characterization studies that may be utilized, for example, to identify, select, and/or characterize vaccine candidates or vaccine compositions (e.g., manufacturing batches thereof), or components thereof as described herein.

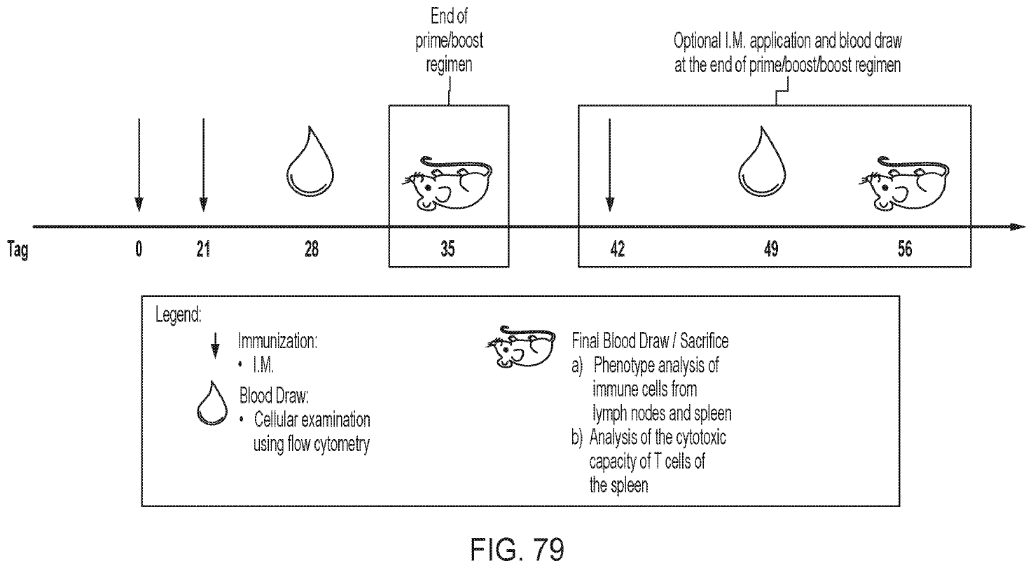

FIG. 79 presents a potential immunization protocol that can be utilized to assess ability of a vaccine candidate that comprises or delivers an antigen(s) as described herein to induce B- and/or T-cells, e.g., after intramuscular immunization, directed to the antigen(s) and/or epitope(s) thereof. In some embodiments, level and/or diversity of response is determined. In some embodiments, presence and/or level of neutralizing antibodies is/are determined. In some embodiments, protection of the immunized subject from challenge with VZV is assessed.

Alternatively or additionally, in some embodiments, one or more in vitro assessments may be performed, for example:
(1) in vitro expression of an antigen encoded by an RNA included in a vaccine composition; and/or
(2) in vitro potency of antigen expressed from an RNA included in a vaccine composition as described herein.

### Example 14: Exemplary Clinical Studies of VZV RNA Vaccine Compositions

The present Example describes certain clinical assessments that may be performed of certain RNA vaccine compositions described herein for treatment and/or prevention of VZV infection and in certain particular embodiments for treatment and/or prevention of VZV latent infection.

In some embodiments, more than one different vaccine candidate may be assessed. In some such embodiments, different candidates may vary, for example in:
1. RNA platform (e.g., unmodified RNA, nucleoside-modified RNA, self-amplifying RNA (saRNA), trans-amplifying RNA);
2. encoded antigen—e.g.,
   a. which VZV protein(s) utilized
   b. full length protein antigen vs fragment vs plurality of fragments vs fusion with one or more heterologous sequences (e.g., membrane tether, secretion, linker(s))
   c. epitopes from different (and/or multiple) phases of VZV life cycle
3. number of RNAs
4. non-coding elements of RNA construct
   a. cap and/or cap-adjacent sequences
   b. 5′UTR
   c. 3′ UTR
   d. polyA tail
5. lipid composition of LNP.

In one particular embodiment, up to three candidate vaccines that have only 1 mRNA encoding for a VZV glycoprotein (e.g., VZV gE, gI, gB, gH, gK, gL, gC, gN, or gM, or any glycoprotein described in Table 1A or 1B) or a VZV tegument protein are evaluated and/or up to three candidates that contain 2 mRNAs, one encoding for a VZV glycoprotein (e.g., VZV gE, gI, gB, gH, gK, gL, gC, gN, or gM, or any glycoprotein described in Table 1A or 1B) or a VZV tegument protein or variants and another one encoding for CD8 and/or CD4 epitopes from conserved antigens (and optionally considering conserved T-cell epitopes from various stages of the VZV life cycle) are evaluated. In this particular exemplary embodiment, vaccine candidates may be evaluated by intramuscular administration, for example, based on a dose-escalation scheme.

### Example 15: Exemplary Datasets for Cytomegalovirus (CMV) and Methods for IEDB Data Curation

The present Example provides exemplary datasets curated for identification of regions and/or epitopes of CMV antigens that may be useful for immunogenic compositions (e.g., vaccine compositions and epitope binding agents, etc.). In some embodiments, such regions and/or epitopes of CMV antigens can be expressed by CMV during a latent phase of its life cycle and/or during an active infection. In some embodiments, such regions and/or epitopes of CMV antigens can be or comprise T cell epitopes. In some embodiments, such regions and/or epitopes of CMV antigens can be or comprise B cell epitopes. In some embodiments, such regions and/or epitopes of CMV antigens can be or comprise predominantly T cell epitopes.

### Exemplary Datasets Curated for Cytomegalovirus (“CMV”)

RNA counts for infected CD14+ cells were obtained from table GSE101341_CD14_umis.txt.gz on Shnayder et al. “Defining the Transcriptional Landscape during Cytomegalovirus Latency with Single-Cell RNA Sequencing.” mBio. 2018; 9(2):e00013-18. Published 2018 Mar. 13. doi:10.1128/mBio.00013-18), the entire contents of which are incorporated herein by reference for the purposes described herein. To summarize data per gene, cells with more than 10 UMI aligning of viral genes were chosen. After, mean UMI across remaining cells was counted and presented as a “count” per gene.

RNA sequencing for CD34+ cells was obtained from Supplementary Table 2 of Cheng et al. “Transcriptome-wide characterization of human cytomegalovirus in natural infection and experimental latency.” Proc Natl Acad Sci USA. 2017; 114(49):E10586-E10595. doi:10.1073/pnas.1710522114, the entire contents of which are incorporated herein by reference for the purposes described herein. Mean FPKM of SS-enriched and clinical samples was taken.

Immunopeptidomics: Data from PXD0131201 (Lübke et al. “Identification of HCMV-derived T cell epitopes in seropositive individuals through viral deletion models.” J Exp Med. 2020; 217(3):jem.20191164. doi:10.1084/jem.20191164) and PXD0072032 (Erhard et al. “Improved Ribo-seq enables identification of cryptic translation events.” Nat Methods. 2018; 15(5):363-366. doi:10.1038/nmeth.4631) was downloaded from PRIDE protein repository and re-searched using Spectrum Mill software package (version BI.07.04.210) against all UCSC Genome Browser genes (January 2018), common contaminants, and CMV viral databases specific to each study obtained from UniProt Consortium (PXD013120: strain AD169, proteome ID UP000008991; PXS007203: strain Merlin, proteome ID UP000000938). Searches included oxidated methionine set as a variable modification, and a minimum scored peak intensity of 50% & PSM FDR estimate <1% was used to filter results. All virus-derived sequences between 7 and 17 amino acids in length were considered, resulting in 272 unique identified epitopes. Of these, 35 were not previously identified in the associated publications or reported in the immune epitope database (IEDB). The entire contents of the aforementioned documents are incorporated herein by reference for the purposes described herein.

Protein expression methods: data from Table 73 describing proteins derived from CMV identified in a multiplexed protein expression analysis was downloaded from Weekes et al. “Quantitative temporal viromics: an approach to investigate host-pathogen interaction.” Cell. 2014; 157(6):1460-1472. doi:10.1016/j.cell.2014.04.028), the entire contents of which are incorporated herein by reference for the purposes described herein.

T cells co-cultured with peptide+APC were evaluated for cell proliferation via CFSE. Peptides synthesized following NetMHCPan predictions of full CMV genome. See Lee et al. “Antigen identification for HLA class I- and HLA class II-restricted T cell receptors using cytokine-capturing antigen-presenting cells.” Sci Immunol. 2021; 6(55):eabf4001. doi:10.1126/sciimmunol.abf4001, the entire contents of which are incorporated herein by reference for the purposes described herein.

Ligandomics HHV6B data was downloaded from MassIVE protein repository (MSV000083546) and re-searched using Spectrum Mill software package (version BI.07.04.210) against all UCSC Genome Browser genes (January 2018), common contaminants, and HHV-6B viral database (uniprot-proteome_UP000006930.fasta). Searches included oxidated methionine set as a variable modification, and a minimum scored peak intensity of 50% & PSM FDR estimate <1% was used to filter results. Homology between HHV-6 and HCMV was determined from table 1 of Nicholas J. “Determination and analysis of the complete nucleotide sequence of human herpesvirus.” J Virol. 1996; 70(9):5975-5989. doi:10.1128/JVI.70.9.5975-5989.1996 and from list retrieved online from https://hhv-6foundation.org/research/genes-proteins. The entire contents of the aforementioned documents are incorporated herein by reference for the purposes described herein.

### IEDB Data Curation Process:

MHC ligands, T-cell epitopes, and B-cell epitopes were collected from IEDB from each virus, for example, including the downloadable datasets mhc_ligand_full.csv, tcell_full_v3.csv, and bcell_fullv3.csv, respectively. Filtering and data cleaning was performed to ensure these data only contained relevant epitopes, e.g. negative assay results and non-human viral strains were omitted. In order to summarize the number of unique epitope sequences in these data in an accurate, non-redundant manner, epitopes that were reported in unbiased studies (listed below) were removed. Lastly, the remaining epitopes were classified into six categories indicating the assay used and relevant MHC class, if applicable. T-cell epitopes that were not specified to be CD4+ or CD8+ specific were categorized as “Ambiguous T-cell epitopes”.

### Select Unbiased Studies Include:

1. Lübke et al. “Identification of HCMV-derived T cell epitopes in seropositive individuals through viral deletion models.” J Exp Med. 2020; 217(3):jem.20191164. doi: 10.1084/jem.20191164
2. Erhard et al. “Improved Ribo-seq enables identification of cryptic translation events.” Nat Methods. 2018; 15(5):363-366. doi:10.1038/nmeth.4631
3. Becerra-Artiles et al. “Naturally processed HLA-DR3-restricted HHV-6B peptides are recognized broadly with polyfunctional and cytotoxic CD4 T-cell responses.” Eur J Immunol. 2019; 49(8):1167-1185. doi:10.1002/eji.201948126
4. Lee et al. “Antigen identification for HLA class I- and HLA class II-restricted T cell receptors using cytokine-capturing antigen-presenting cells.” Sci Immunol. 2021; 6(55):eabf4001. doi:10.1126/sciimmunol.abf4001

The entire contents of the aforementioned documents are incorporated herein by reference for the purposes described herein.

Tables 6A-6F present relevant gene-level data curated and/or re-analyzed from literature publications including the datasets described above. Such data include, for example, gene expression, protein expression, HLA ligandomics, and immunogenicity. The tables also identify genes that can be useful for a vaccine described herein per systematic analysis and ranking process based on the exemplary curated data noted above.

### Example 16: Exemplary Methods for Assessing Suitability of CMV Genes as Targets for CD8+ and CD4+ T Cell Responses

The present Example provides exemplary methods for identifying CMV genes as targets for CD8+ and/or CD4+ T cell responses. In some embodiments, CMV genes identified as targets for CD8+ and/or CD4+ T cell responses may be useful for immunogenic compositions (e.g., vaccine compositions, and epitope binding agents, etc.). In some embodiments, regions and/or epitopes encoded by CMV genes identified using the methods described herein can be expressed by CMV during a latent phase of its life cycle and/or during an active infection. In some embodiments, such regions and/or epitopes of CMV antigens can be or comprise CD4+ and/or CD8+ T cell epitopes. In some embodiments, such regions and/or epitopes of CMV antigens can be or comprise predominantly CD4+ and/or CD8+ T cell epitopes.

Various lines of evidence were considered to assess the suitability of CMV genes as targets for CD8+ and CD4+ T cell responses. Depending on availability and applicability, RNA expression, protein expression, HLA ligandomics, and/or immunogenicity data from literature publications including the datasets as described in Example 15 were considered. In some embodiments, immunogenicity data was considered to be of higher importance if it was derived from an “unbiased study” (one which assessed genes across the whole viral proteome) than if it was derived from a “biased study” (one which only assessed immunogenicity for a single gene or a subset of genes in the viral proteome). Tables 7A-7B outline a step-wise process by which genes were tiered and/or eliminated from the final roster of selections. Lowest tier number (e.g., “Tier 1”) indicates highest importance. The “Description” column indicates the overall concept of the selection step; whereas the column “Selection Criteria” indicates the specific rule or thresholds used. The column “Genes” lists the genes that correspond to the given priority tier.

### Example 17: Exemplary Methods for CMV Multiple Sequence Alignments

For each protein, strain variants were aligned into a global sequence alignment, for example, using MAFFT (default parameters; https://www.ebi.ac.uk/Tools/msa/mafft/). Cross-strain conservation across each protein sequence was determined, for example, by applying a sliding 9mer window and calculating the frequency of the most frequent 9mer at each position. Furthermore, experimentally defined structures analyzed by the DSSP algorithm (Touw et al. “A series of PDB related databases for everyday needs.” Nucleic Acids Research (2015 January); 43(Database issue): D364-D368; Kabsch and Sander “Dictionary of protein secondary structure: pattern recognition of hydrogen-bonded and geometrical features.” Biopolymers. (1983) 22: 2577-2637. PMID: 6667333; UI: 4128824) were accessed (https://swift.cmbi.umcn.nl/gv/dssp/) and inspected to determine solvent accessibility scores per protein position (likewise using a 9mer rolling average across the protein). Structures were not available for all proteins or all protein regions. The sequence was further annotated by indicating the starting positions of peptides highly scored by T cell epitope prediction tools from the bioinformatics pipeline RECON® (Real-time Epitope Computation for Oncology) (Abelin et al. Immunity (2017) 21:46:315-26; Abelin et al., Immunity (2019) 15; 51:766-779.e17) (percent rank <0.1 for HLA-I; percent rank <0.5 for HLA-II) for alleles that are common in people of European ancestry (A*02:01, A*01:01, A*03:01, A*24:02, A*11:01, B*07:02, B*08:01, B*44:02, B*35:01, B*44:03, C*07:01, C*07:02, C*04:01, C*06:02, C*03:04, DRB1*07:01, DRB1*15:01, DRB1*03:01, DRB1*01:01, DRB1*04:01, DRB4*01:03, DRB3*01:01, DRB3*02:02, DRB5*01:01, DRB3*03:01, DPA1*01:03/DPB1*04:01, DPA1*01:03/DPB1*02:01, DPA1*01:03/DPB1*04:02, DPA1*01:03/DPB1*03:01, DPA1*02:01/DPB1*01:01, DQA1*01:02/DQB1*06:02, DQA1*05:01/DQB1*02:01, DQA1*02:01/DQB1*02:02, DQA1*05:05/DQB1*03:01, DQA1*01:01/DQB1*05:01; these allele selections could be revised to match the allele frequencies applicable in the region(s) of vaccine distribution). Other peptides of interest (those identified by HLA ligandomics studies or otherwise reported in The Immune Epitope Database (iedb.org)) were also positionally located in the global sequence alignments. IEDB peptides were extracted from the tables tcell_full_v3.zip, bcell_full_v3.zip, and mhc_ligand_full.zip as accessed on Jul. 27, 2021. The entire contents of the aforementioned documents are incorporated herein by reference for the purposes described herein.

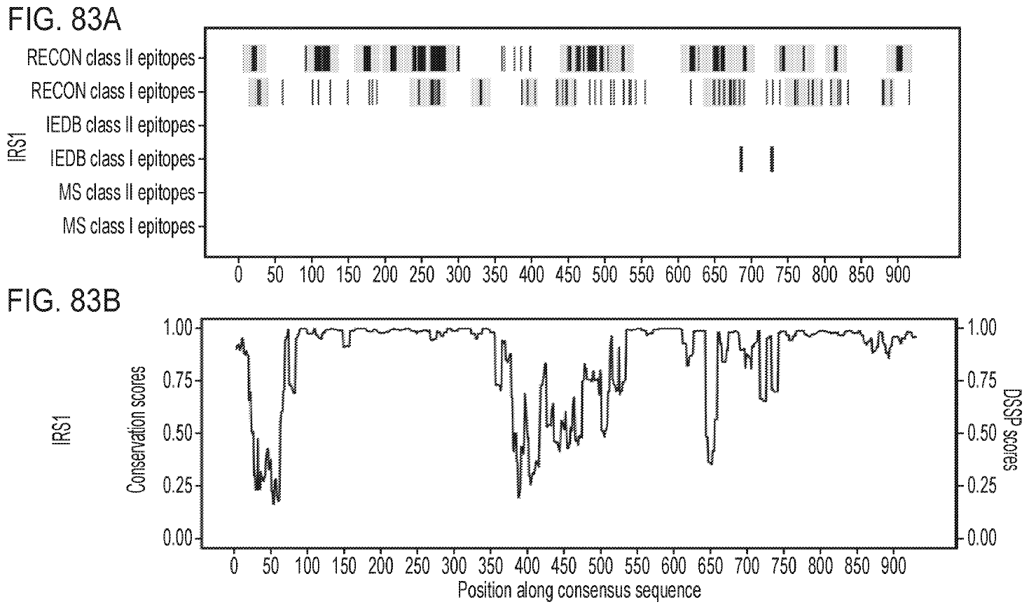

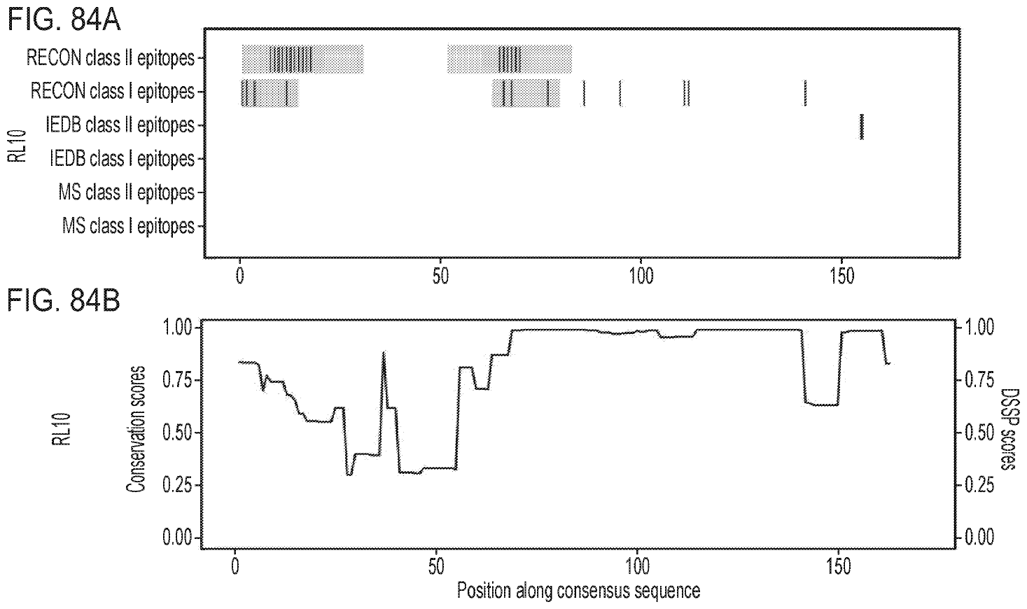

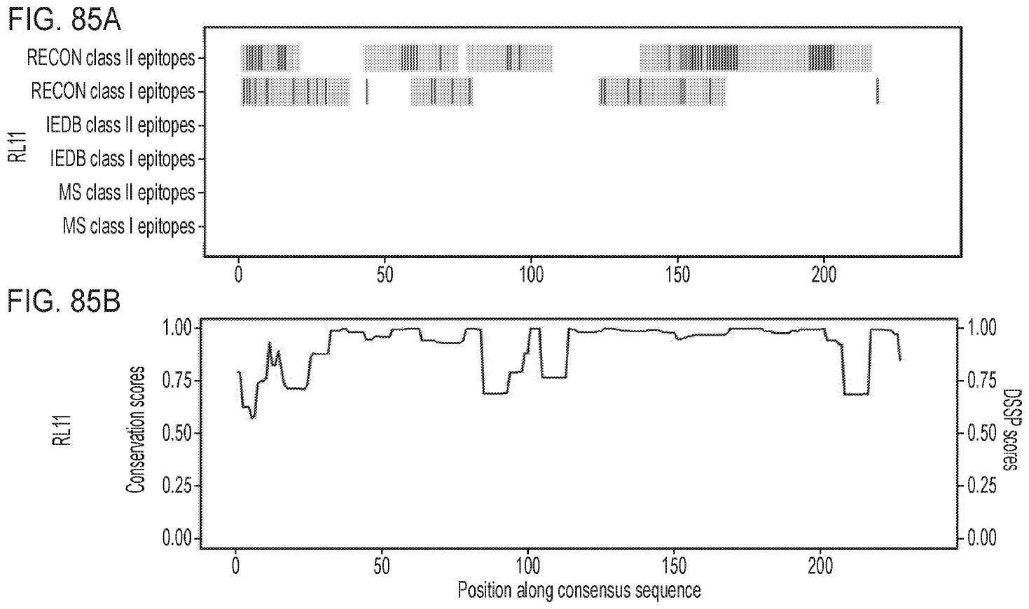

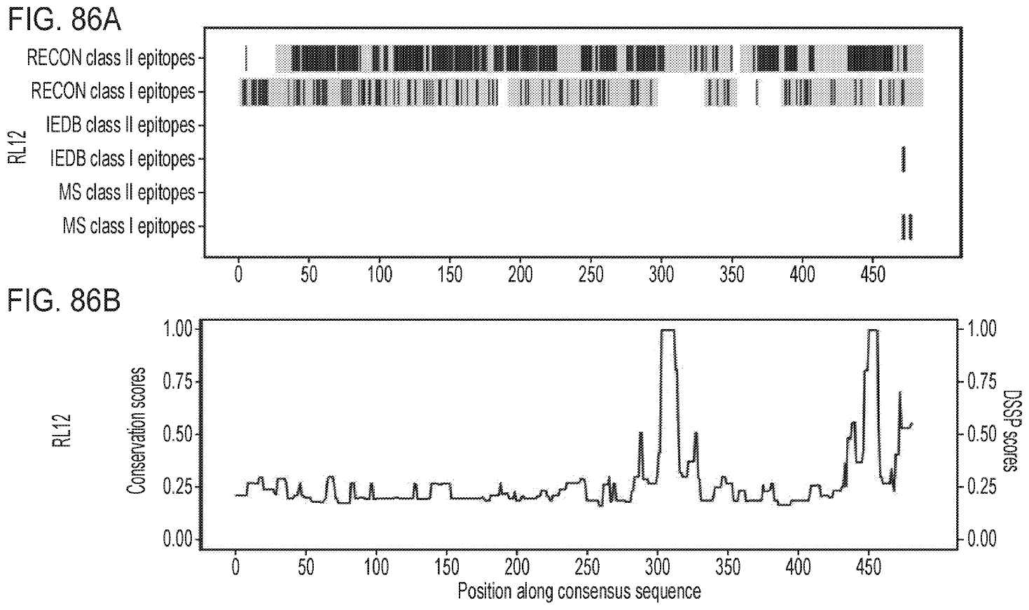

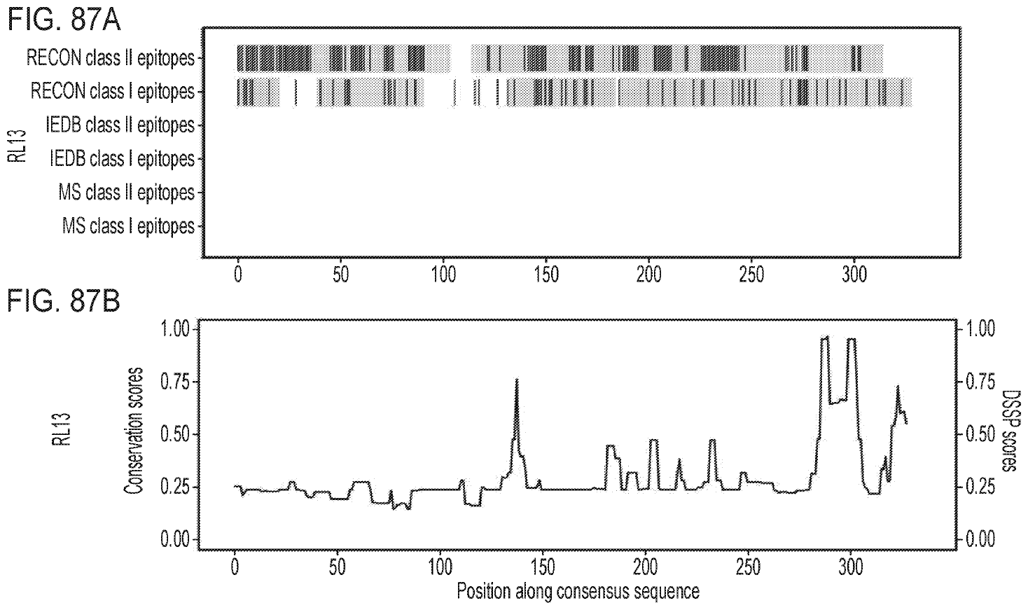

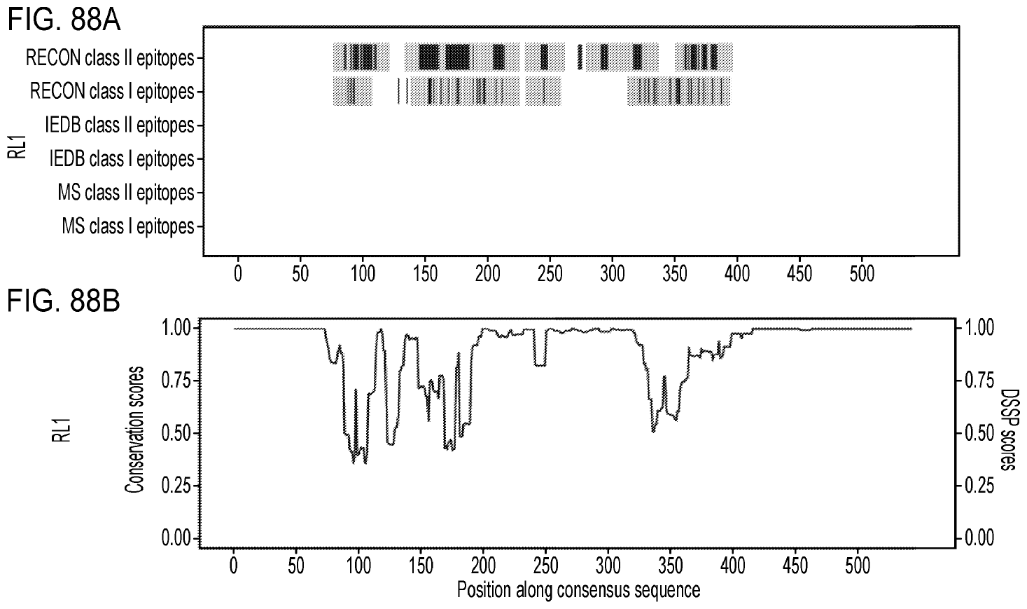

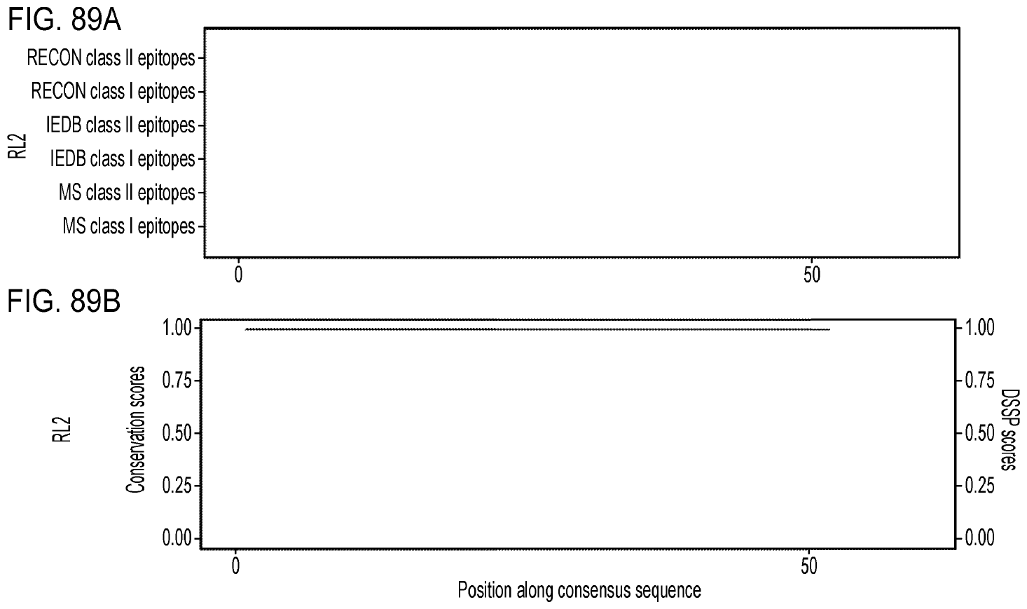

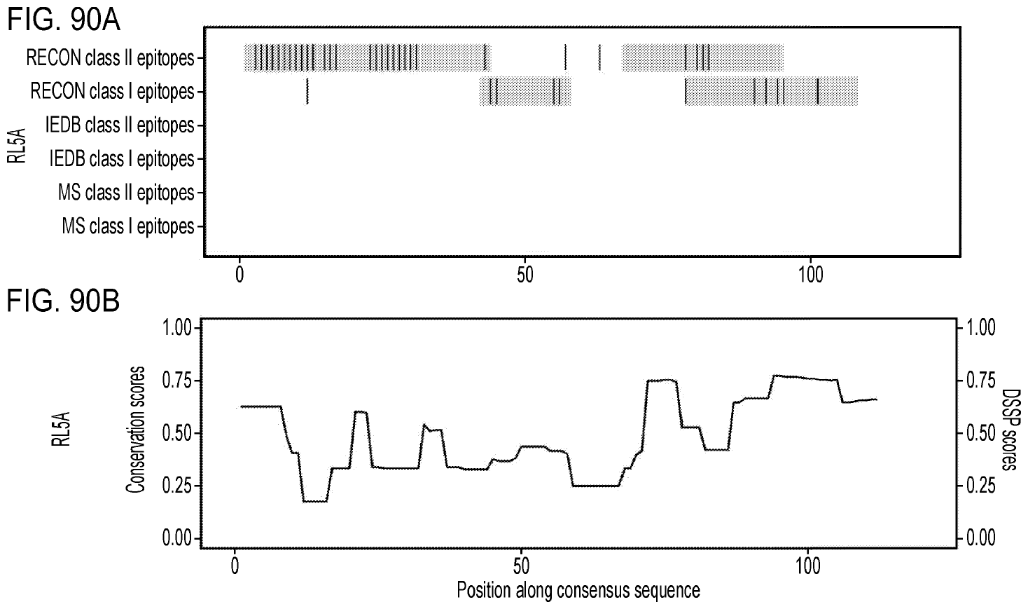

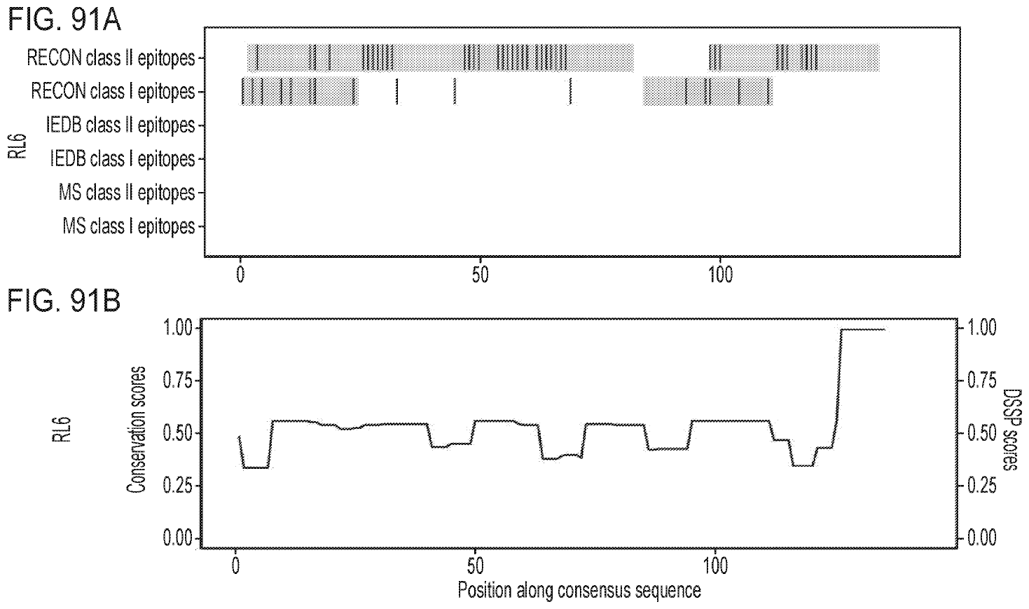

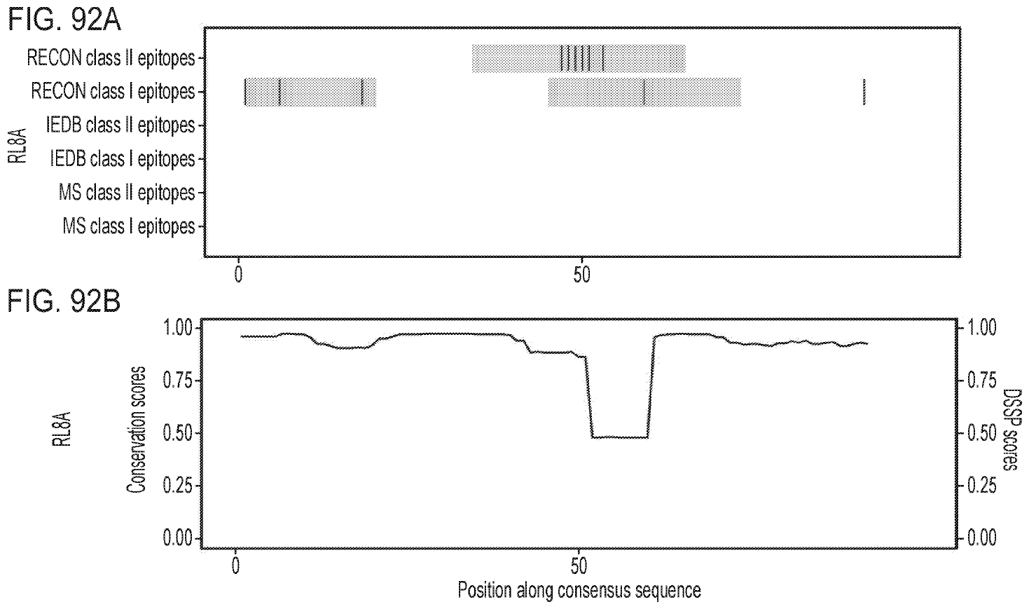

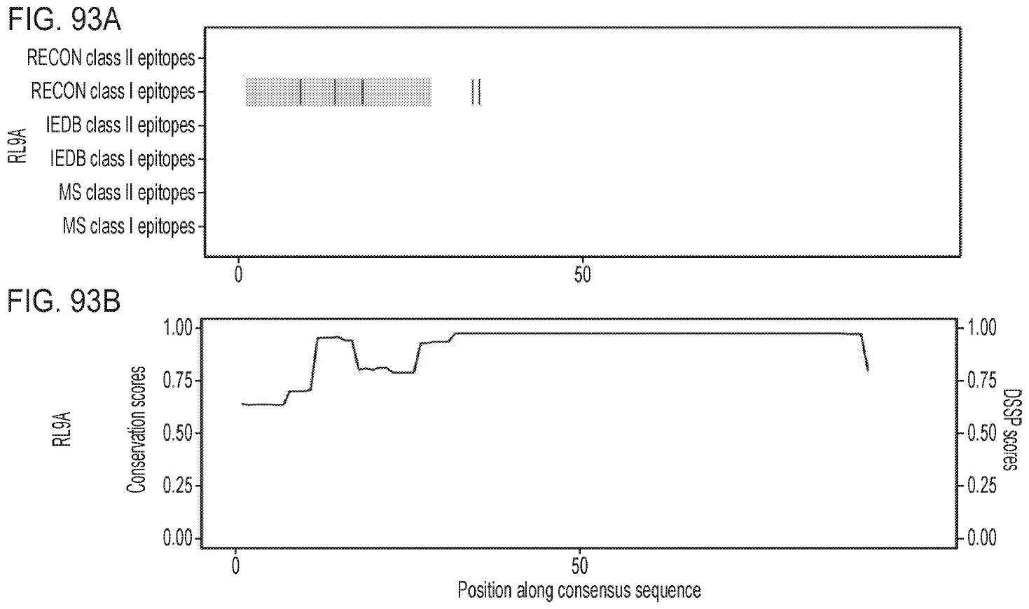

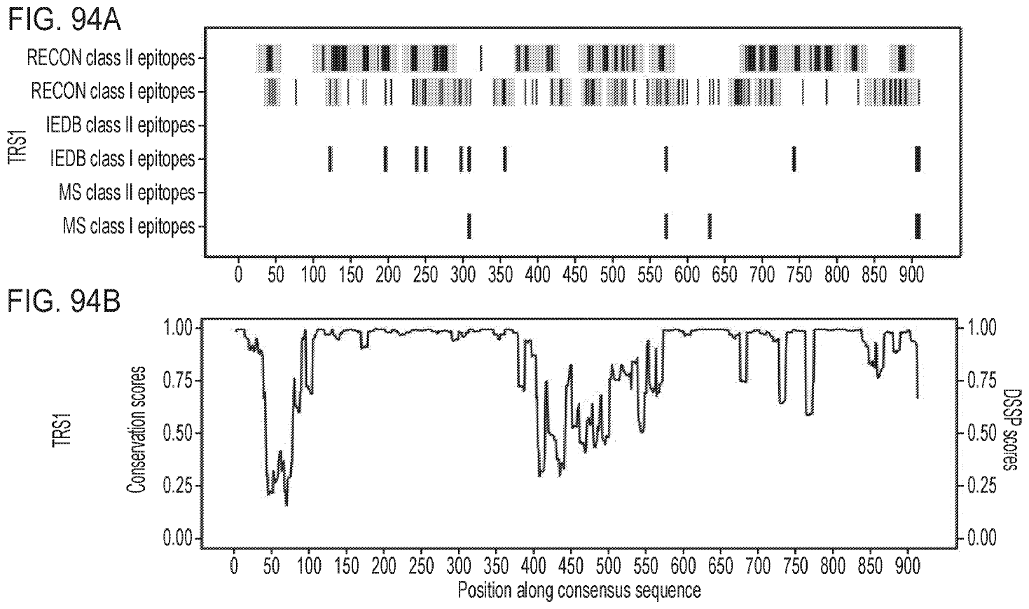

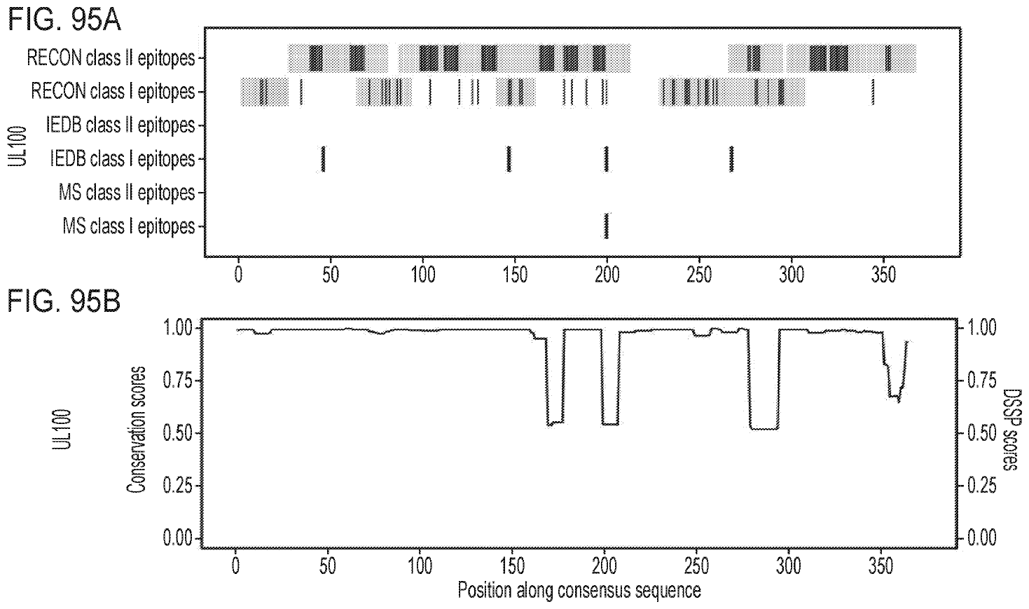

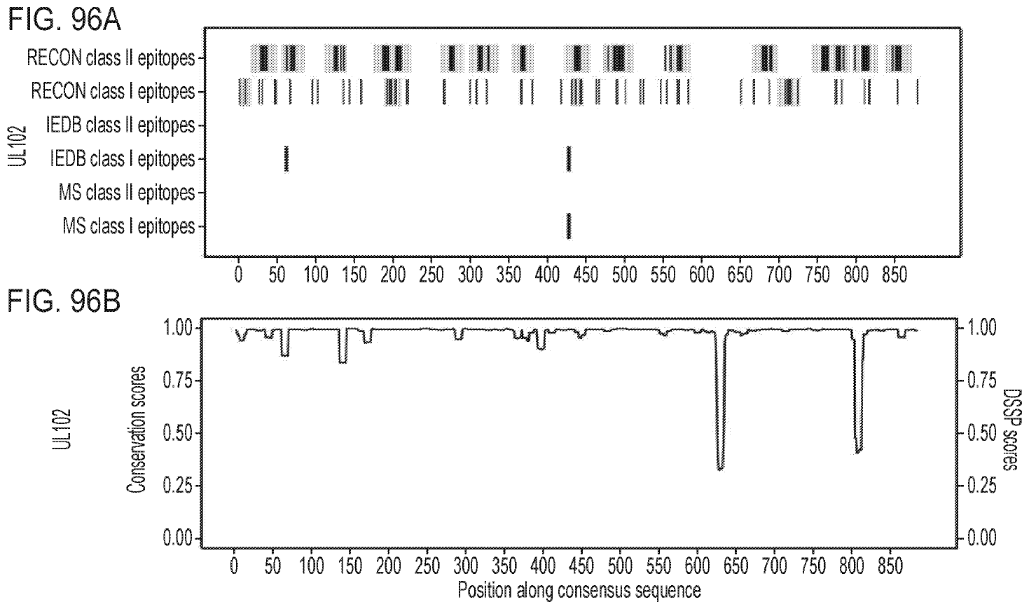

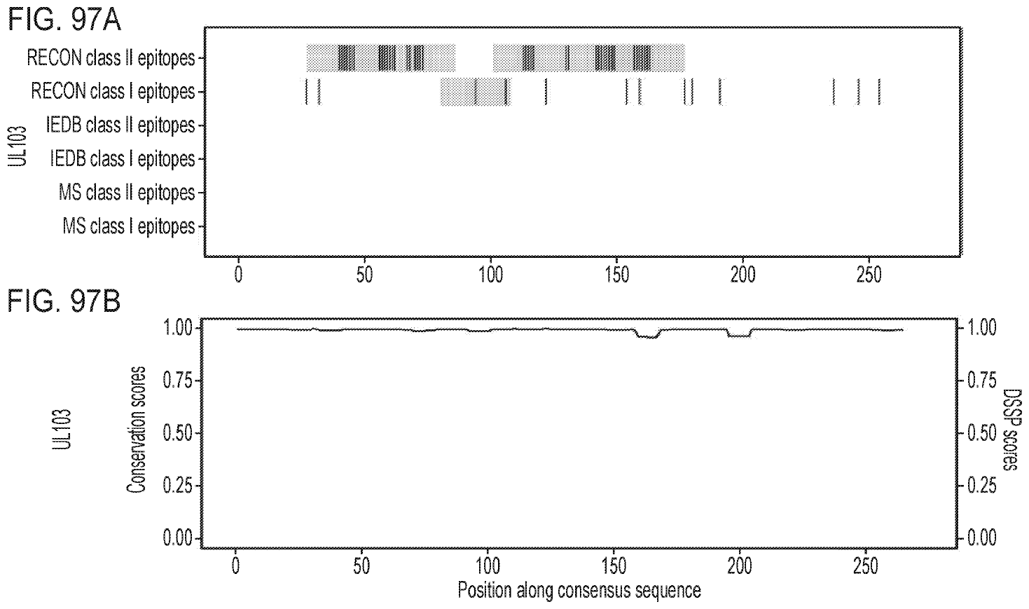

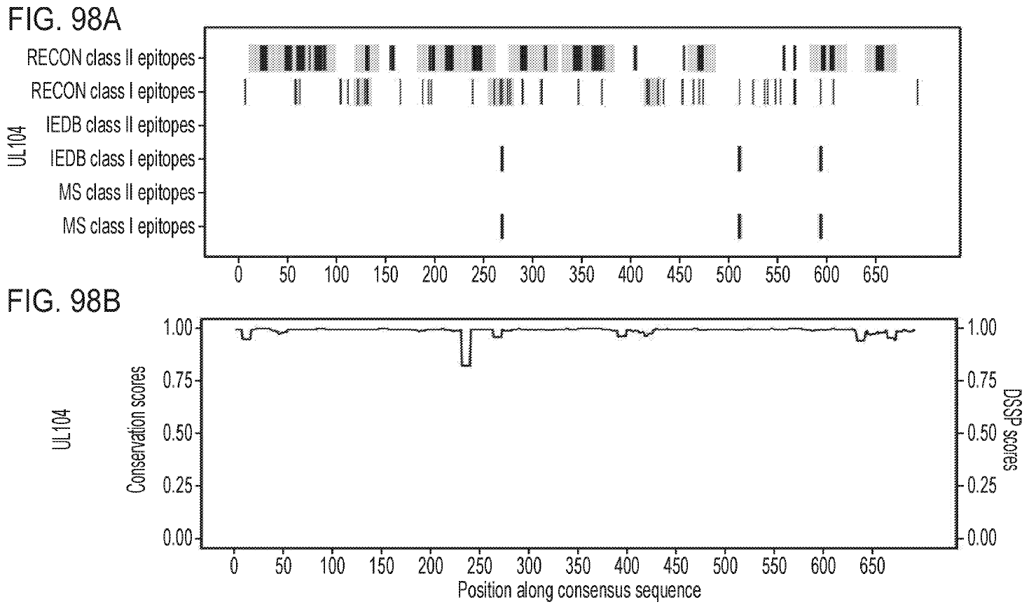

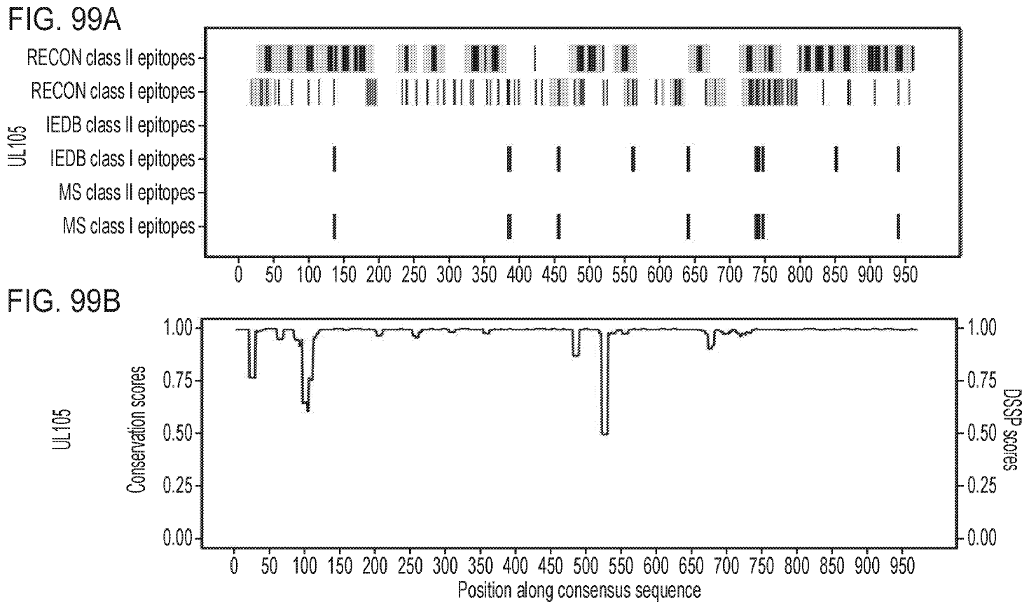

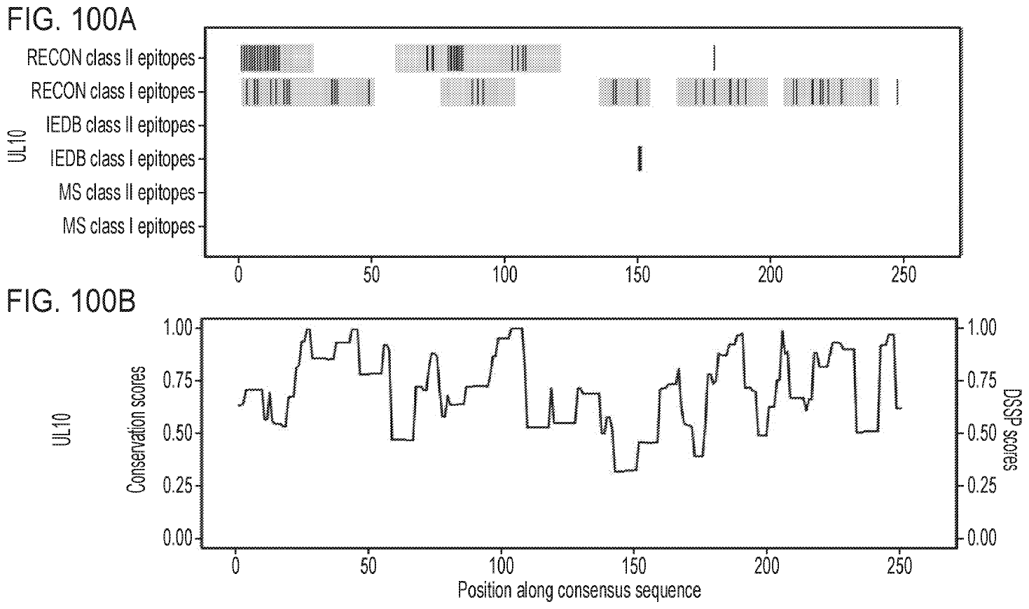

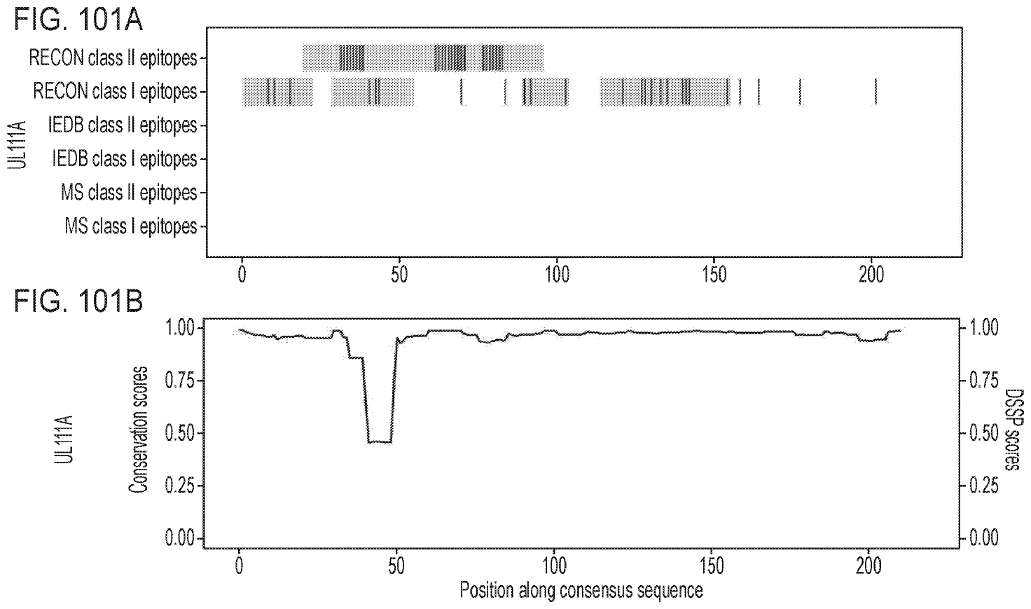

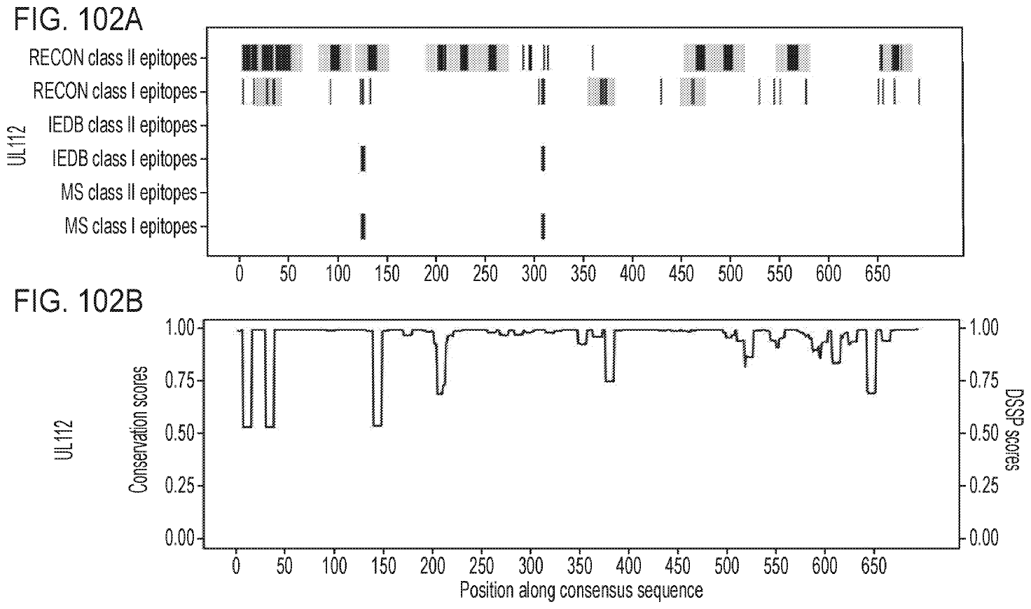

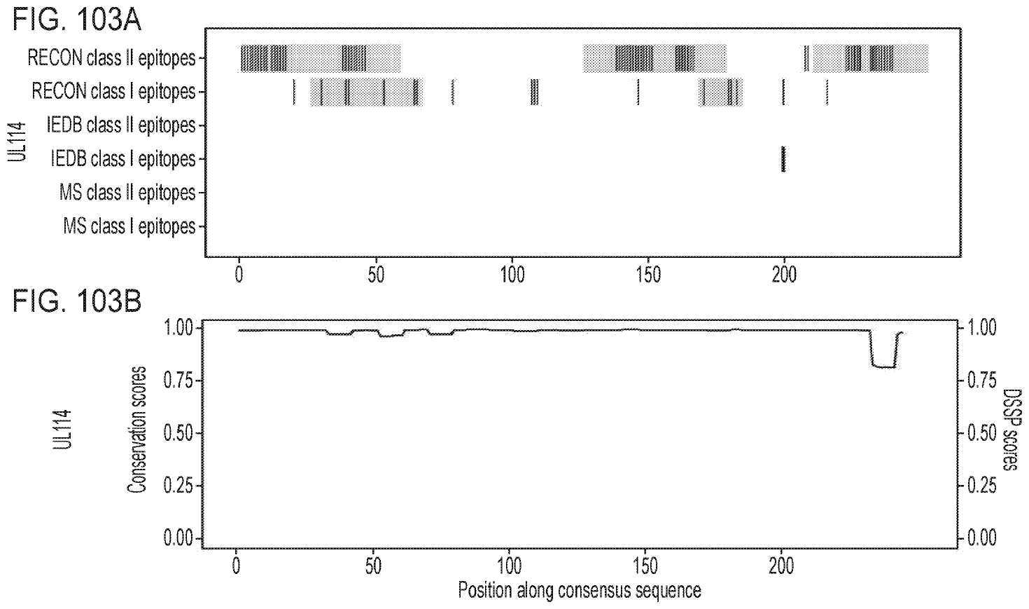

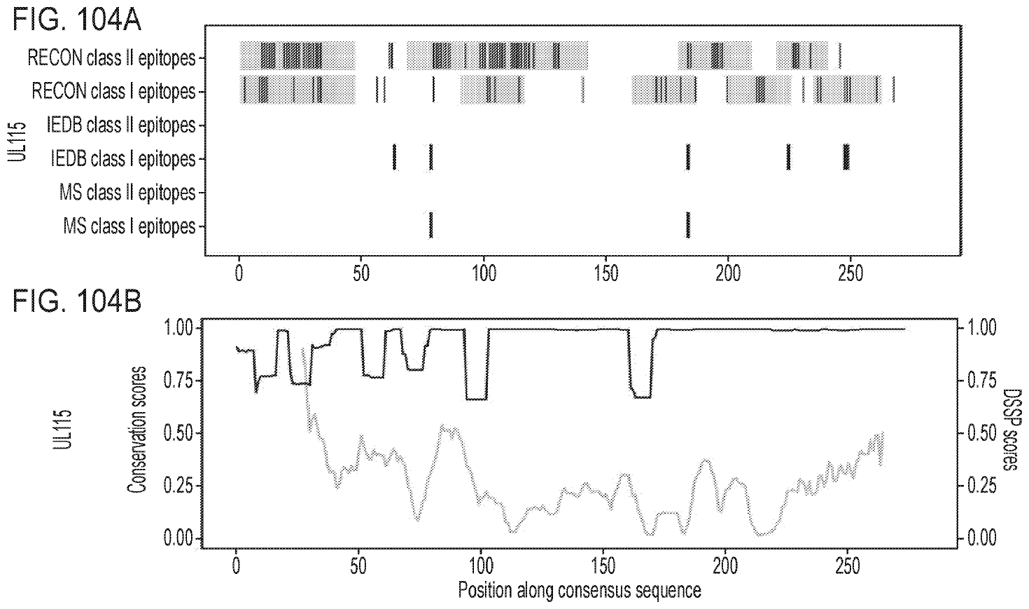

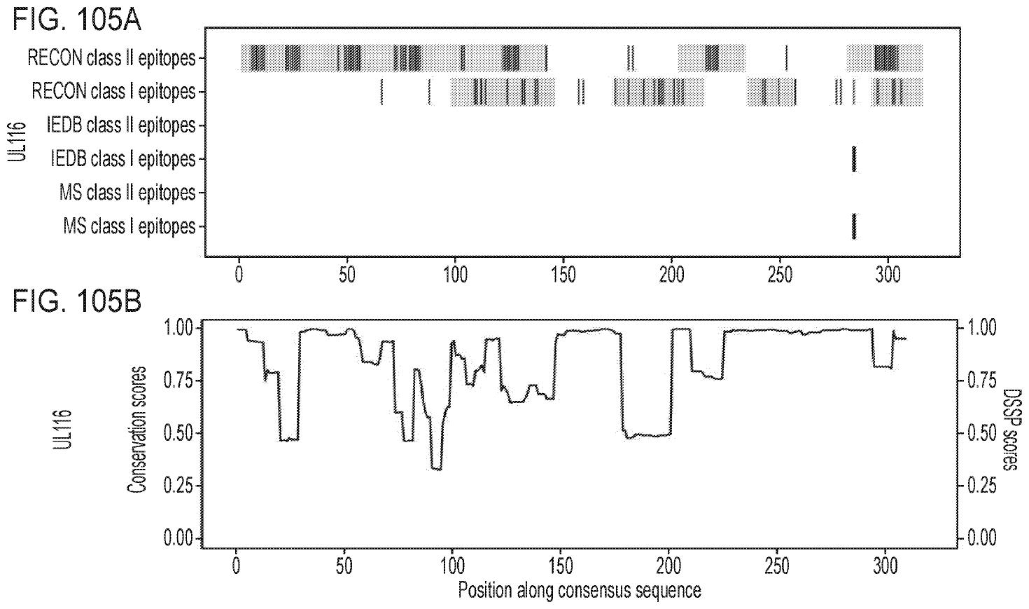

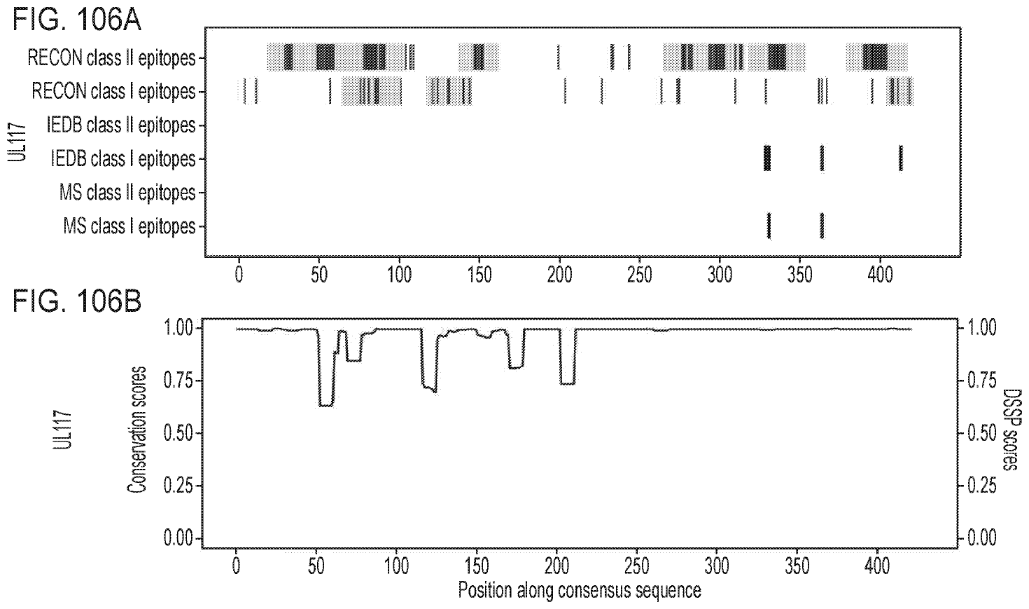

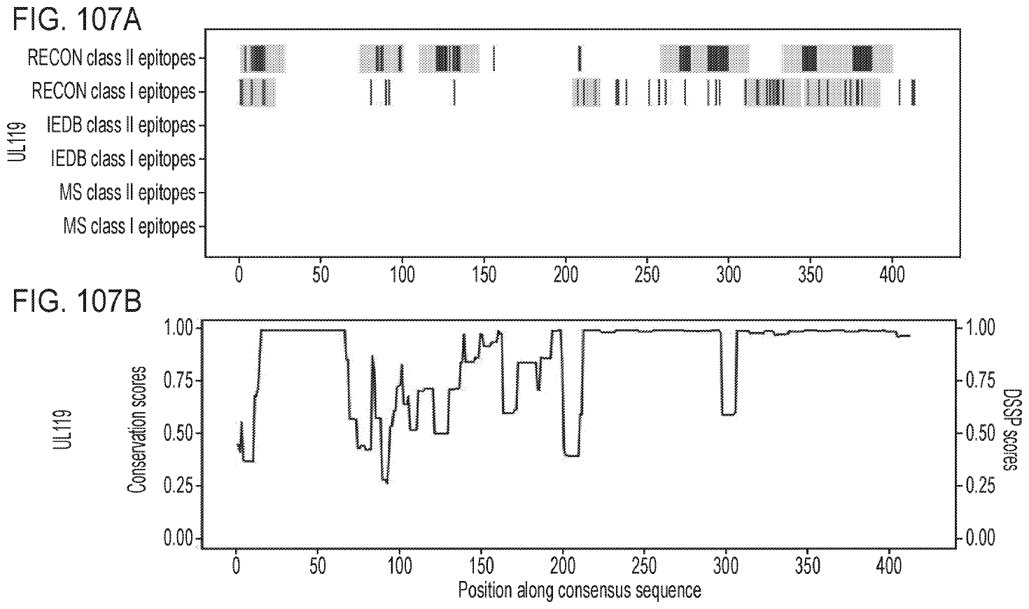

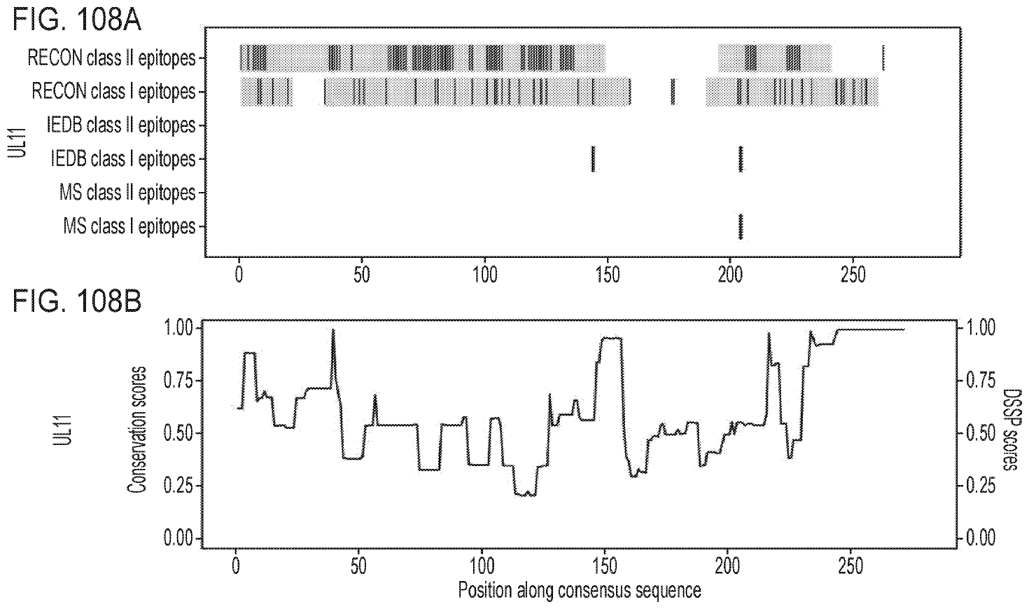

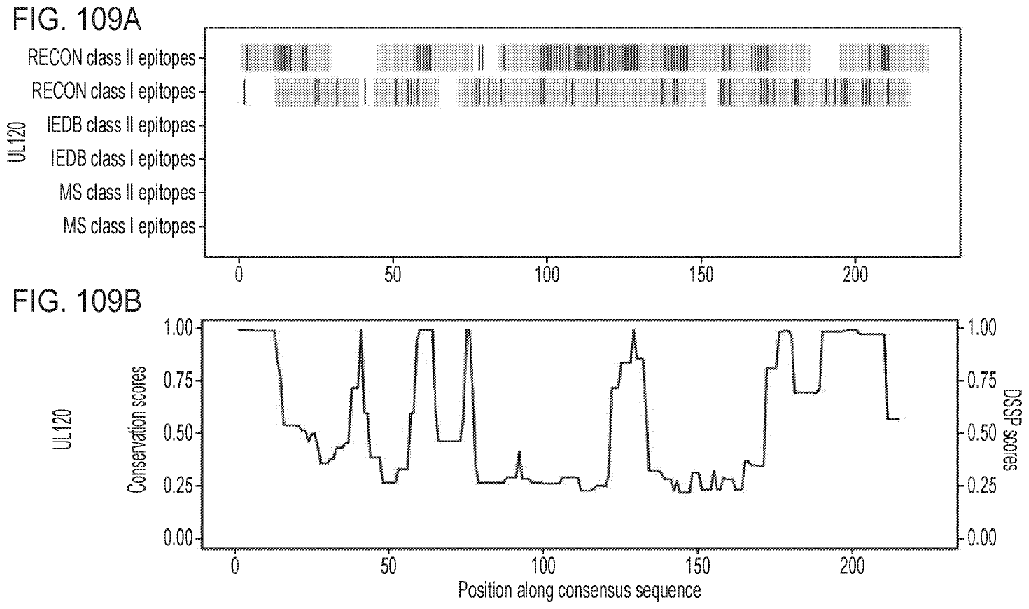

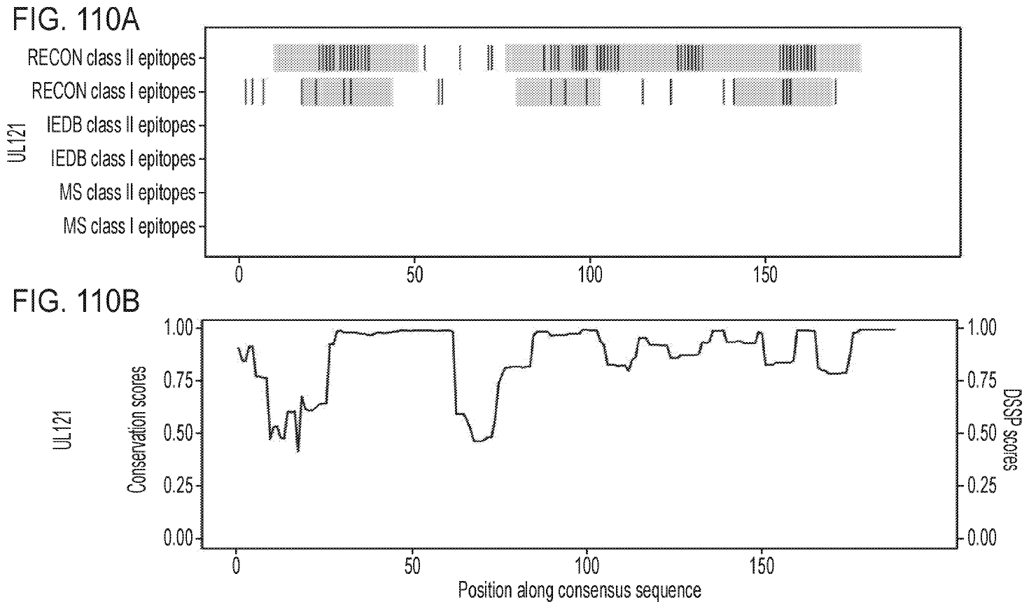

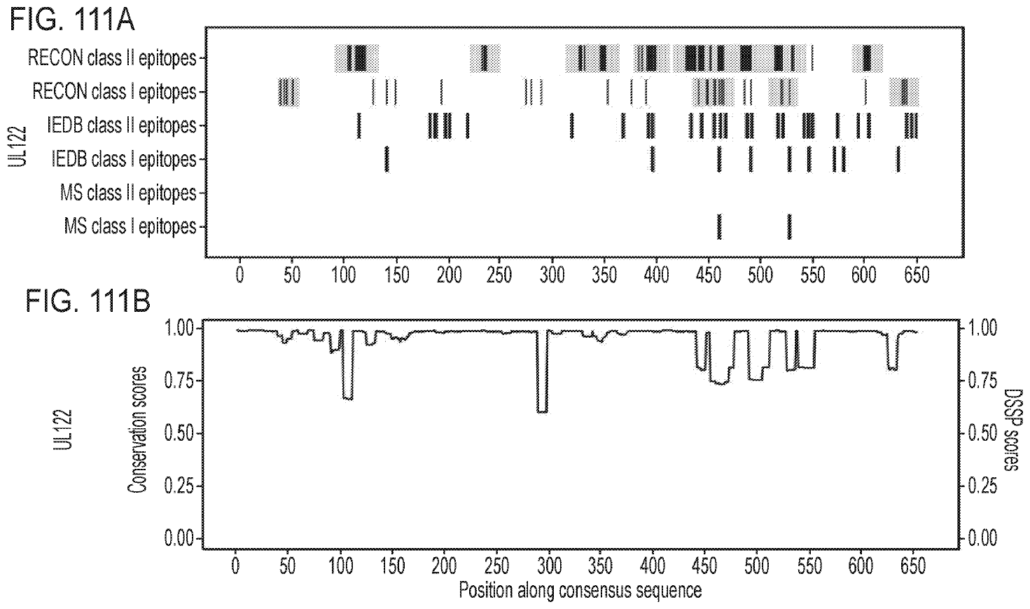

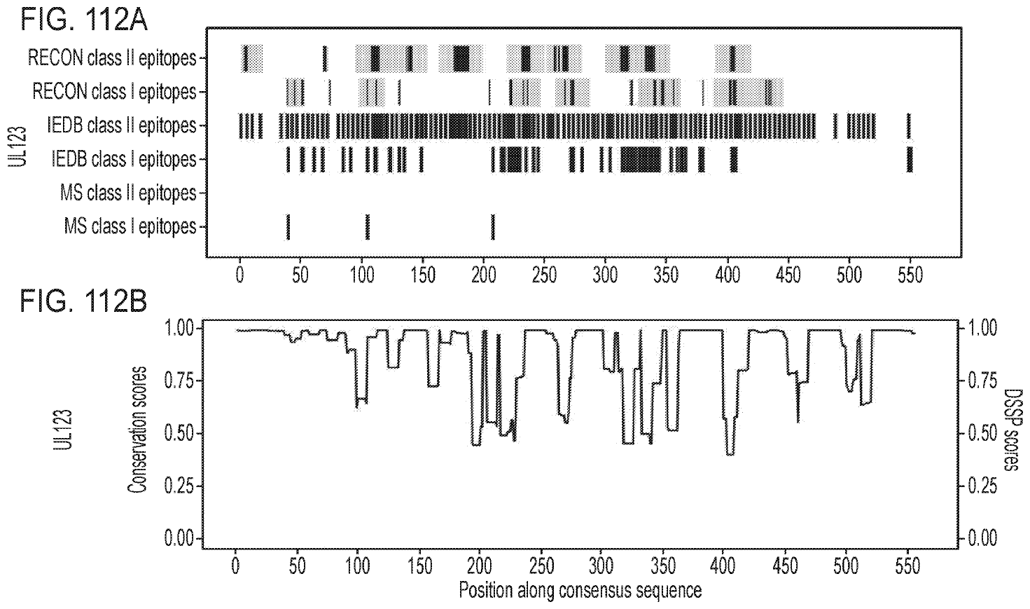

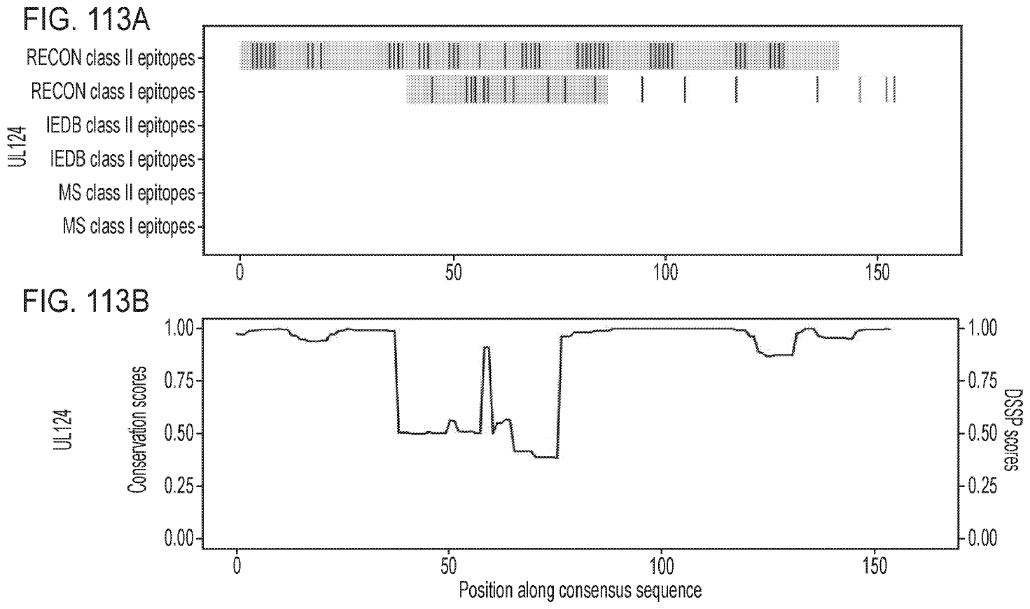

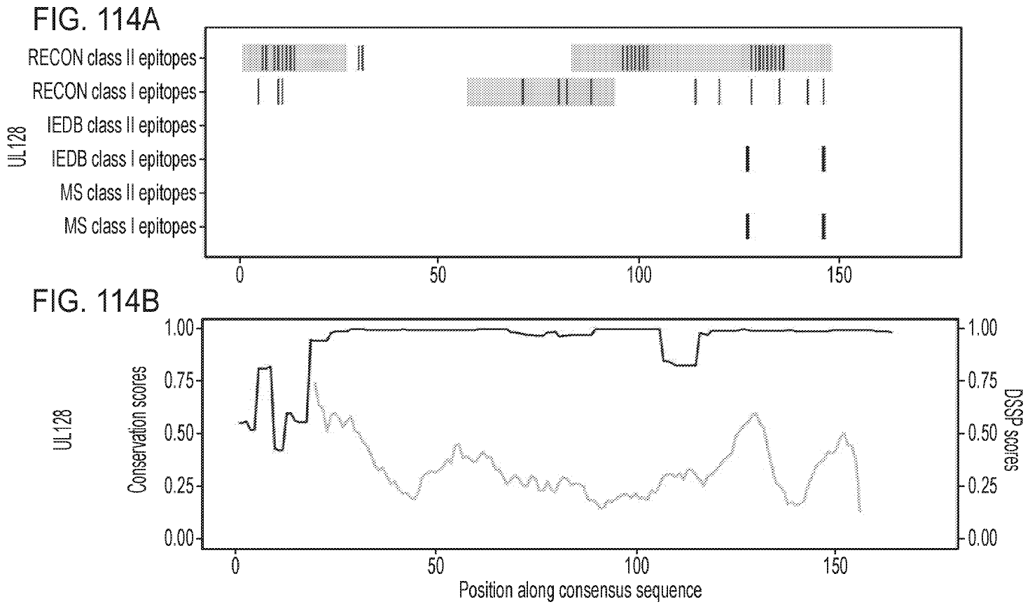

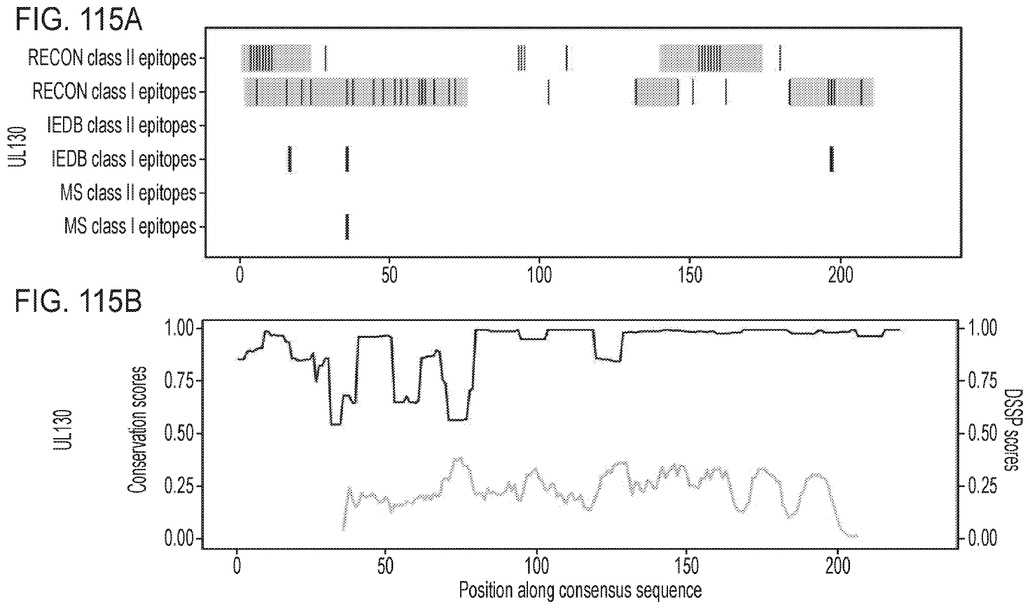

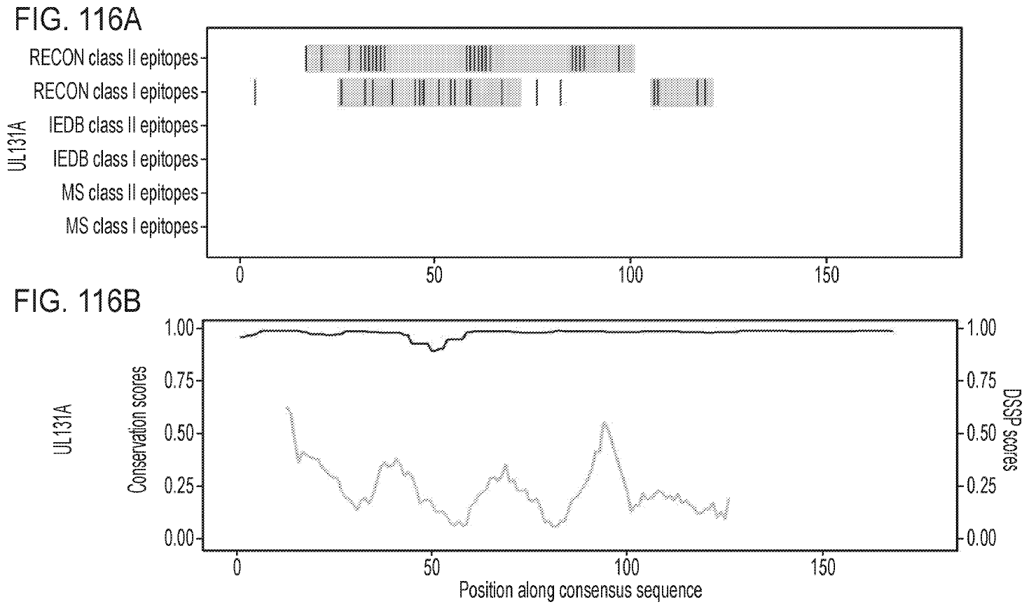

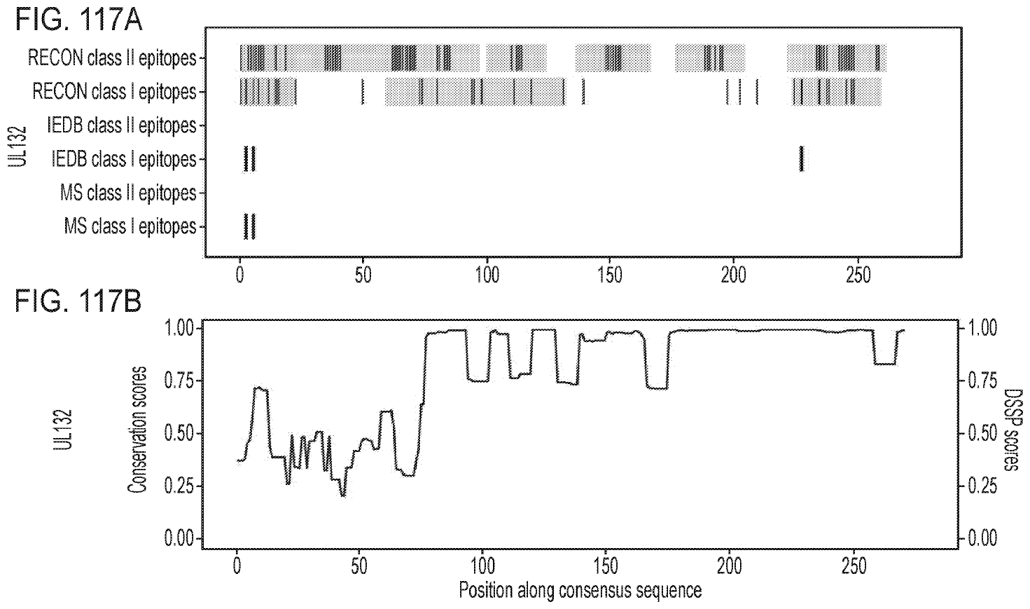

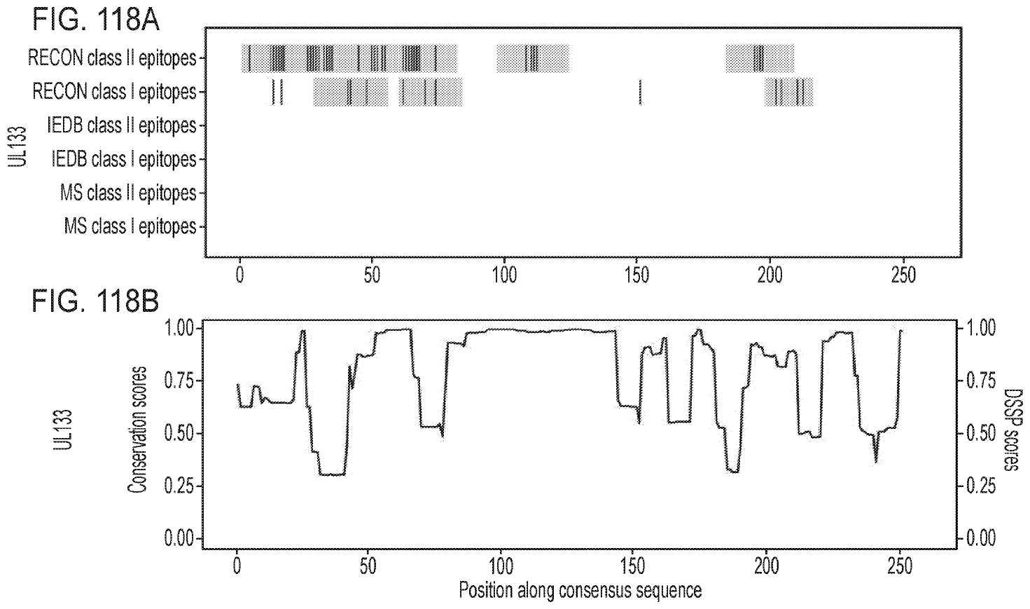

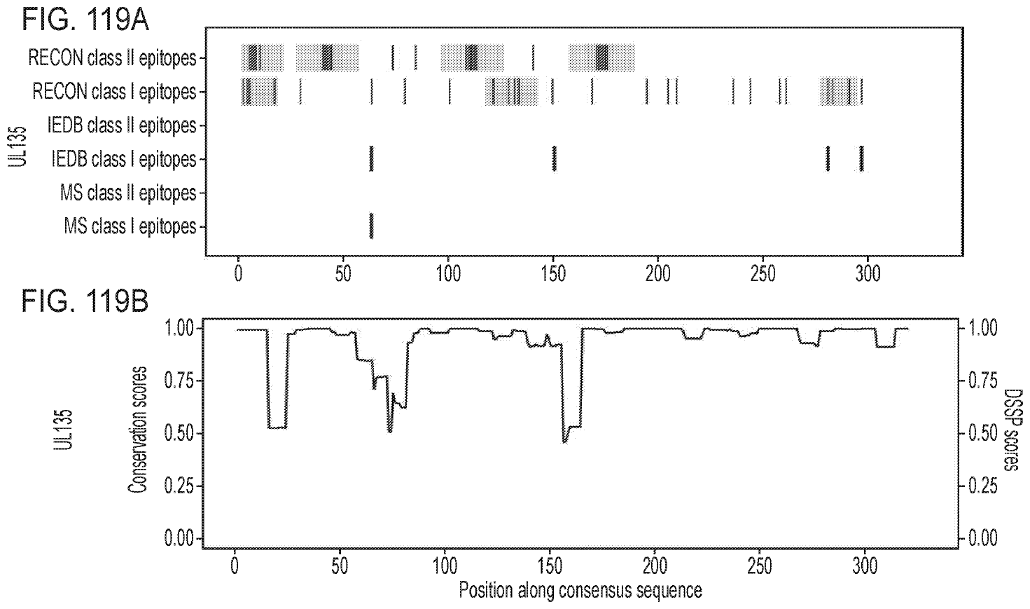

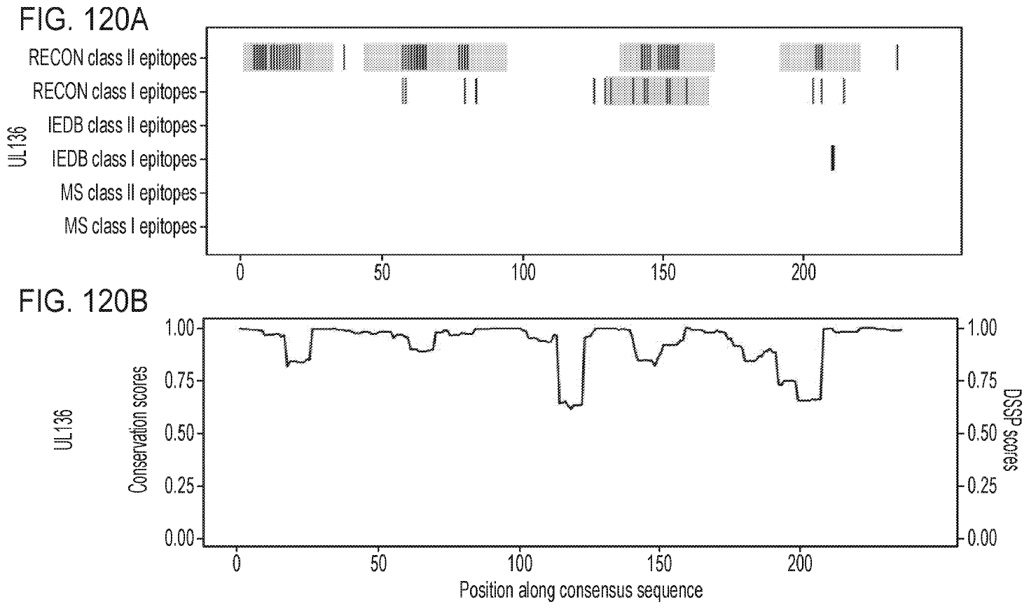

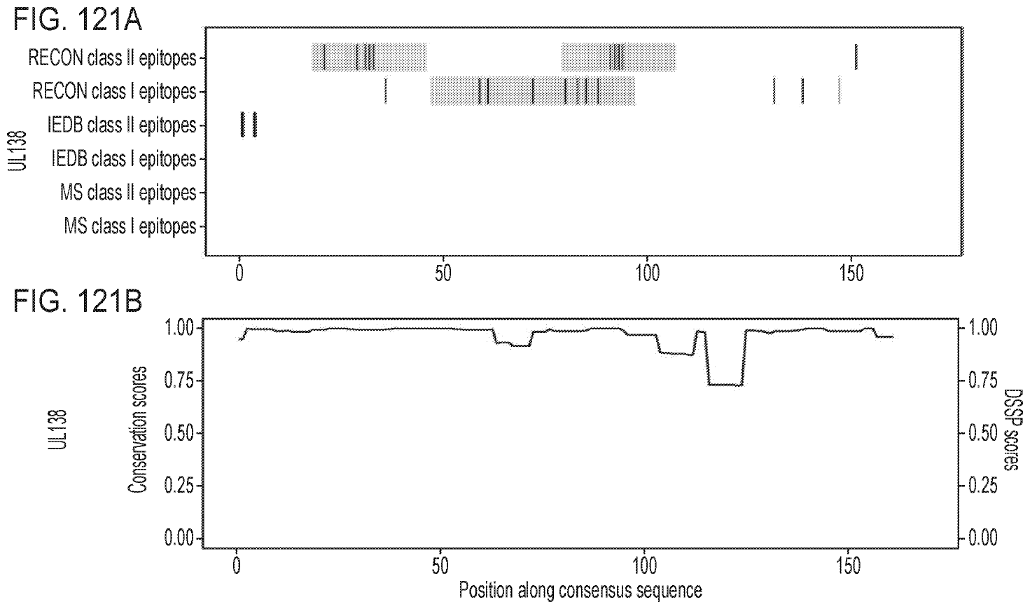

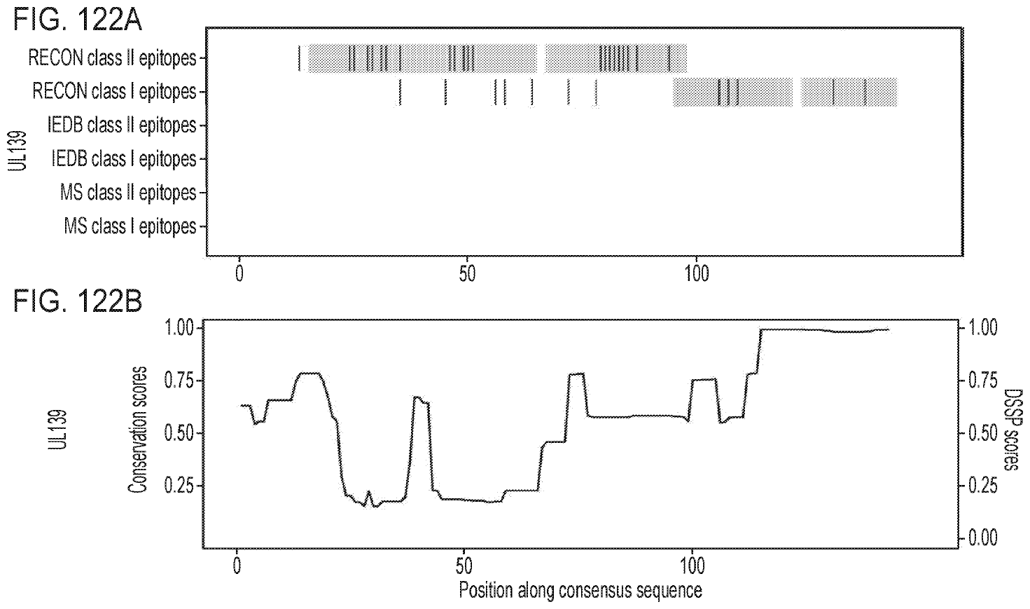

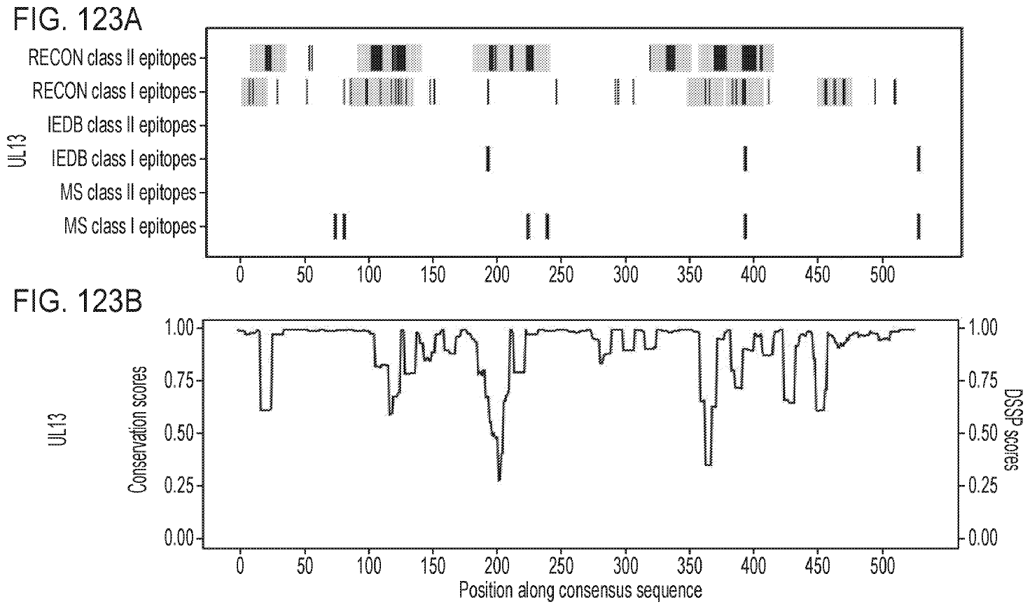

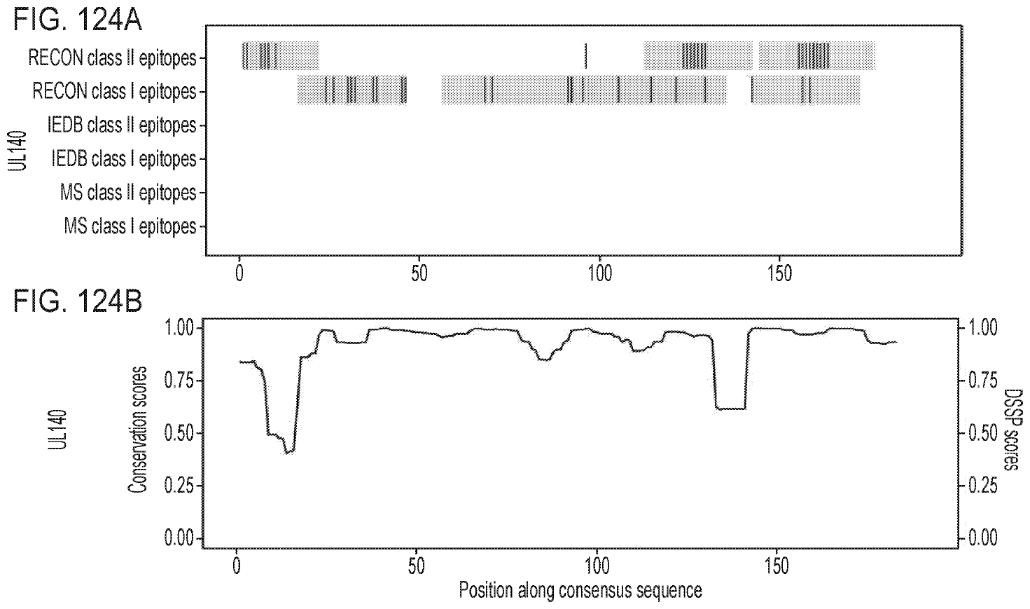

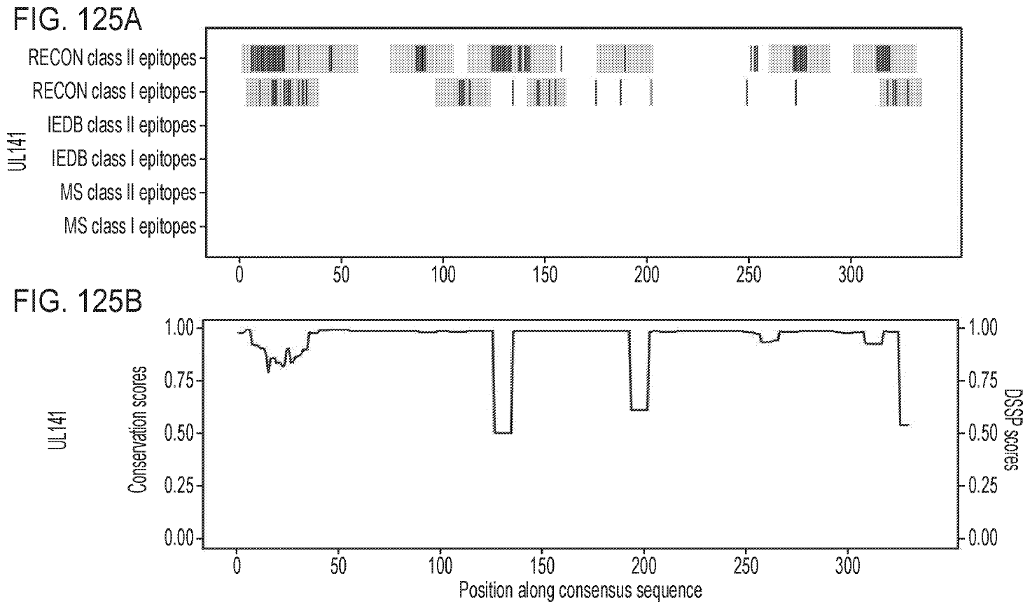

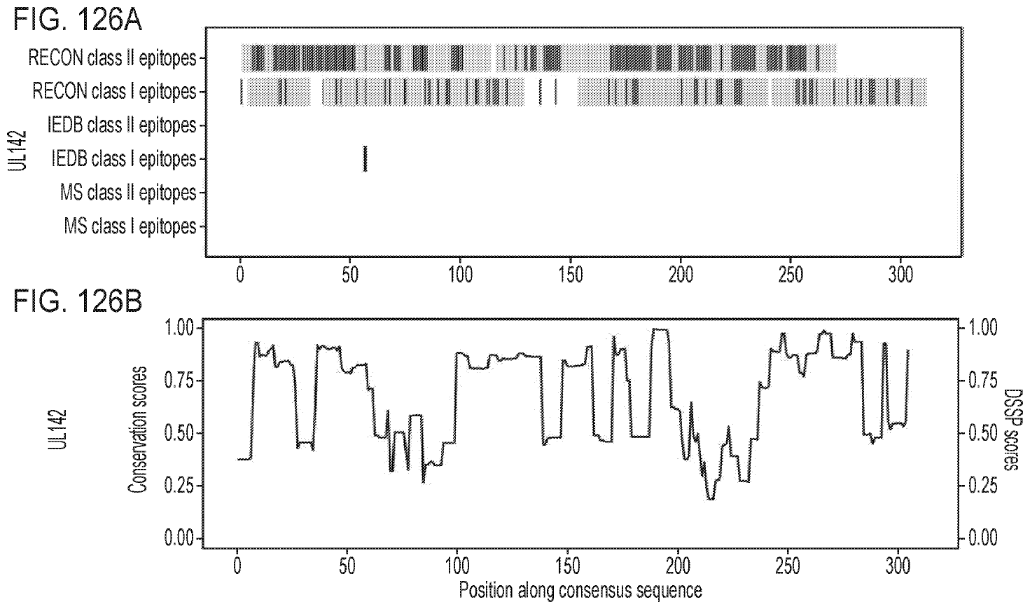

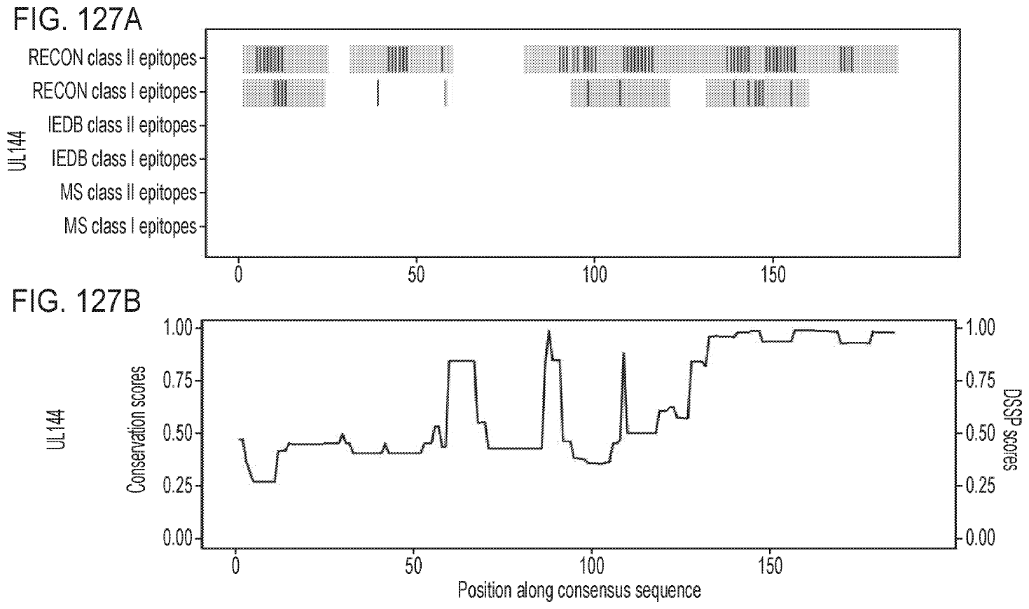

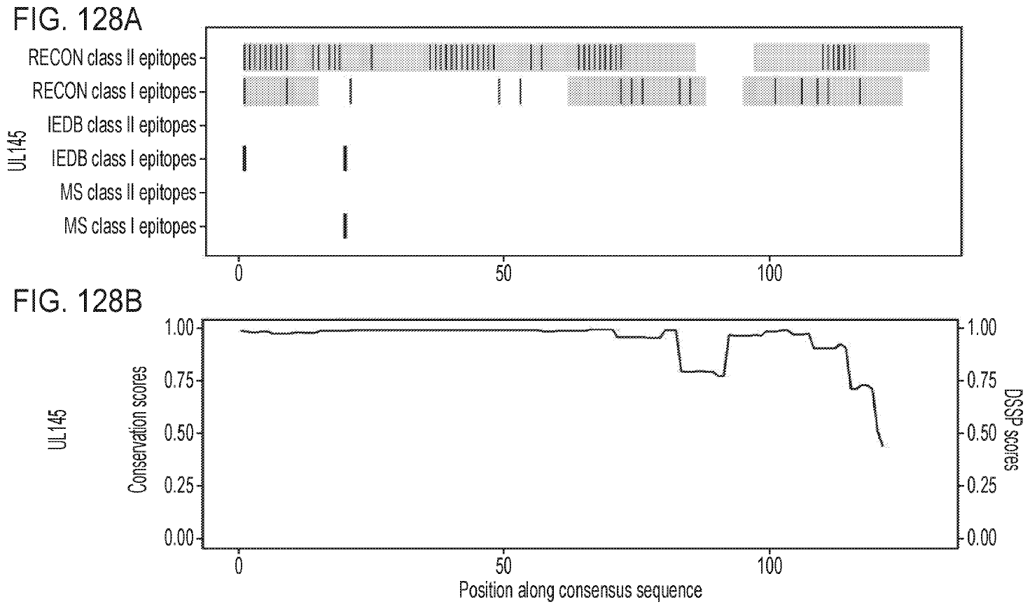

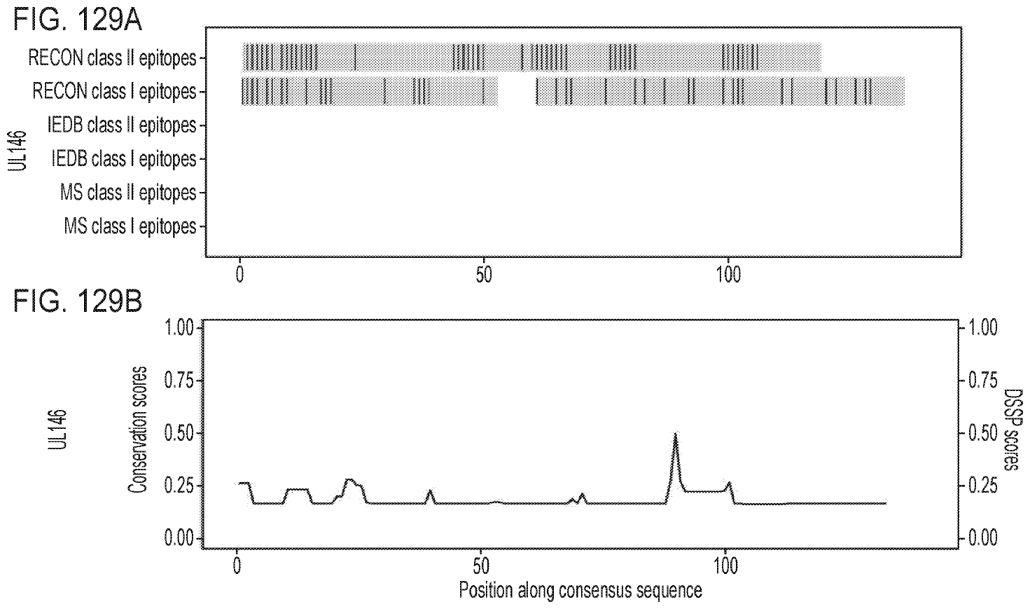

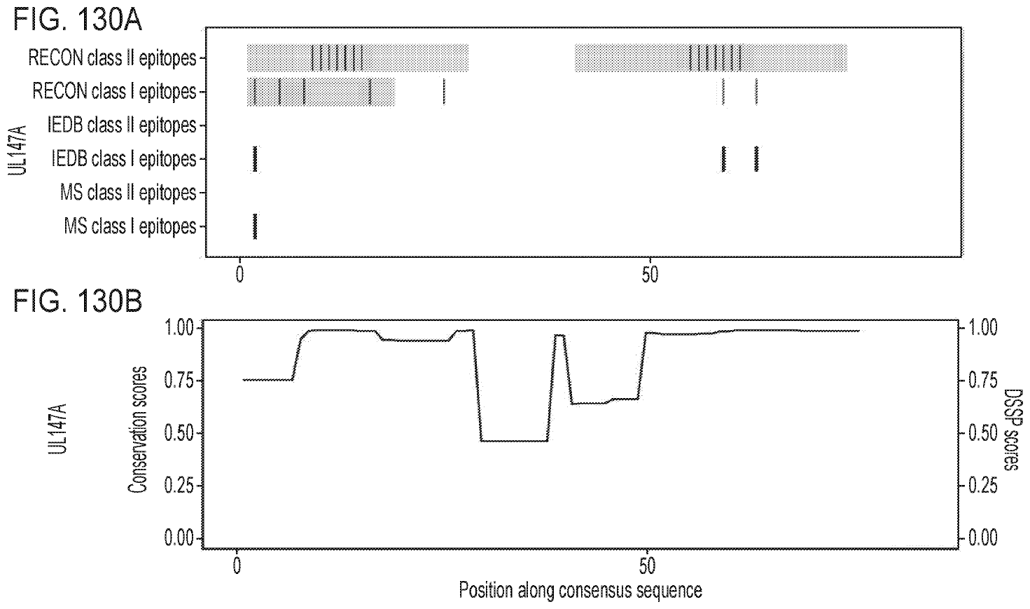

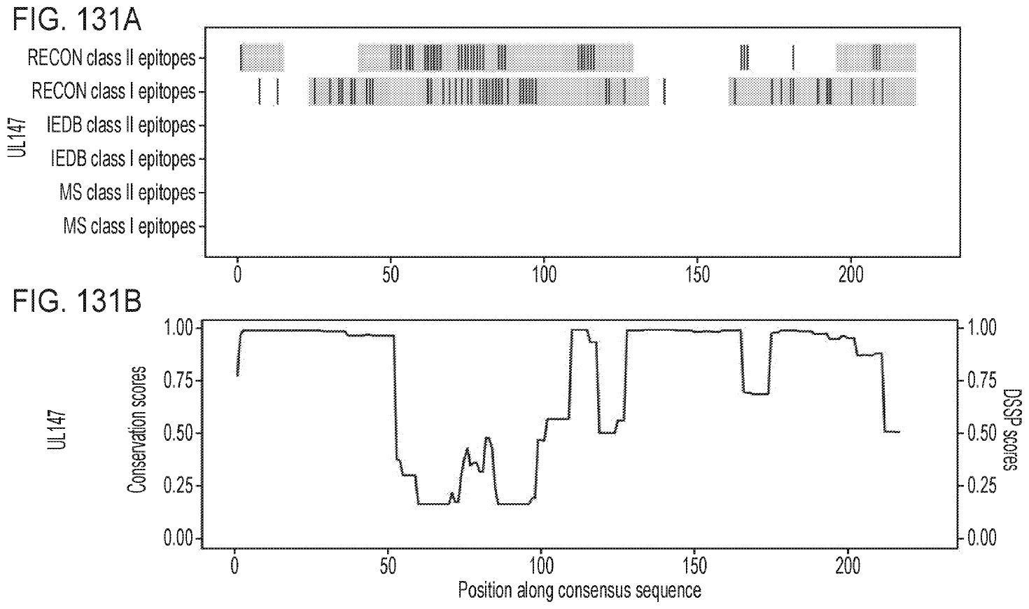

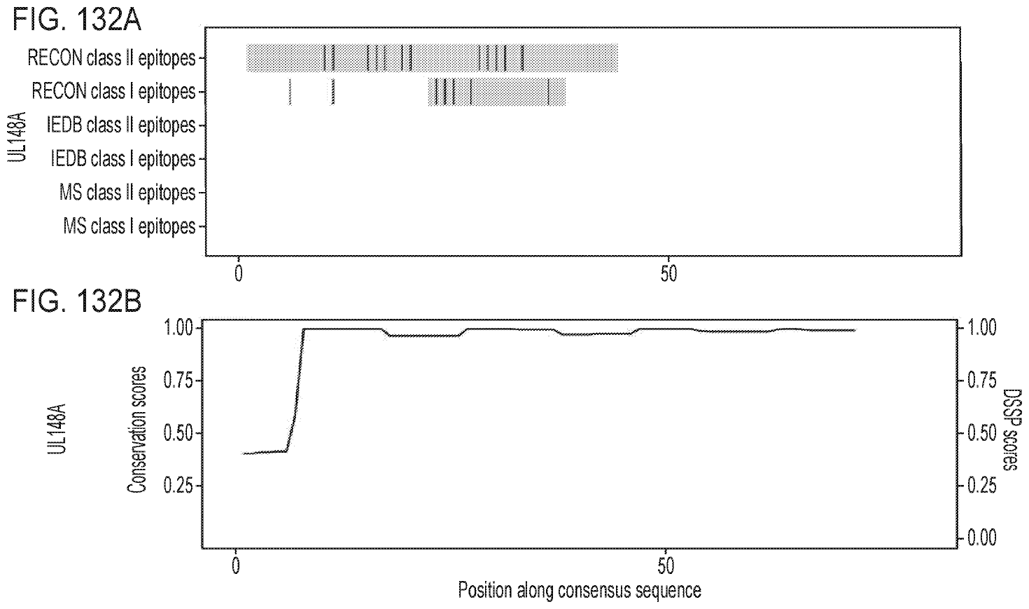

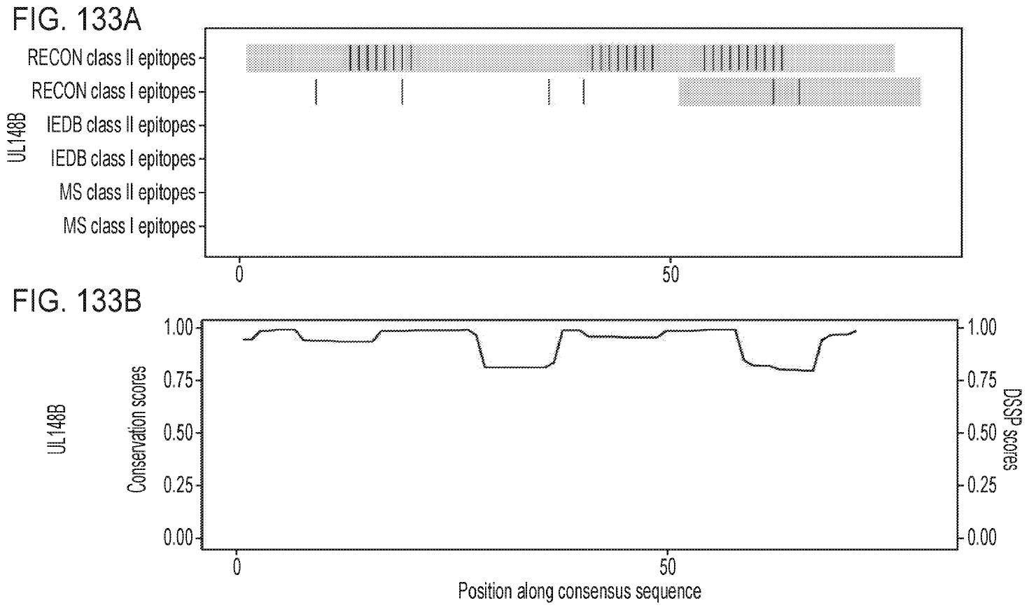

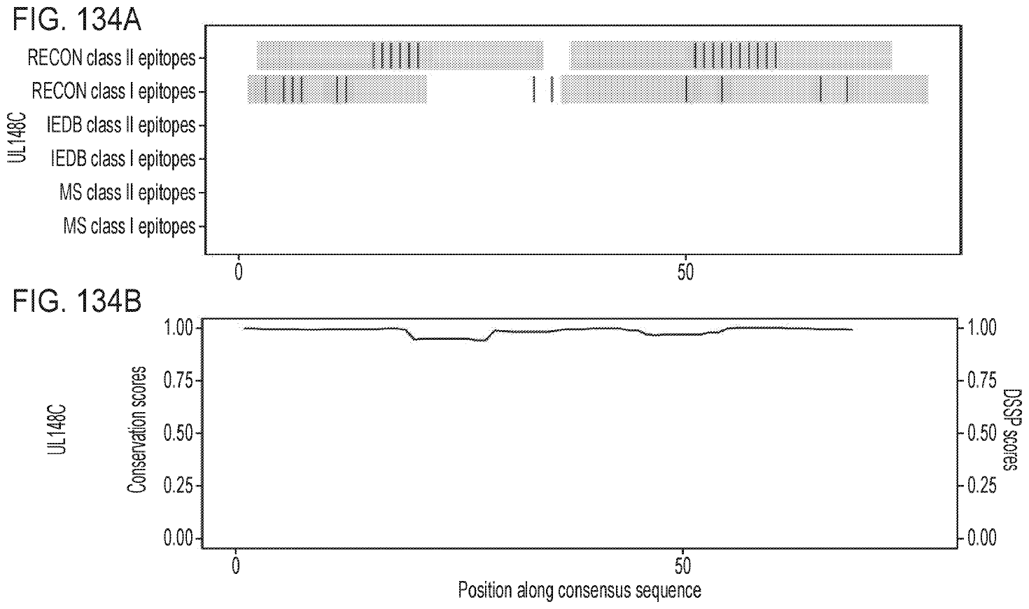

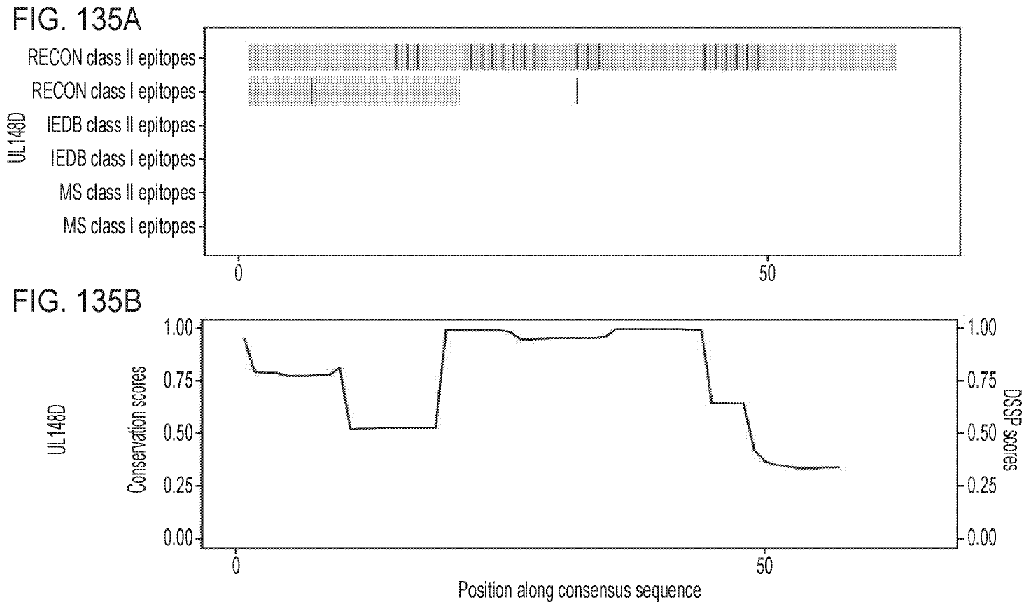

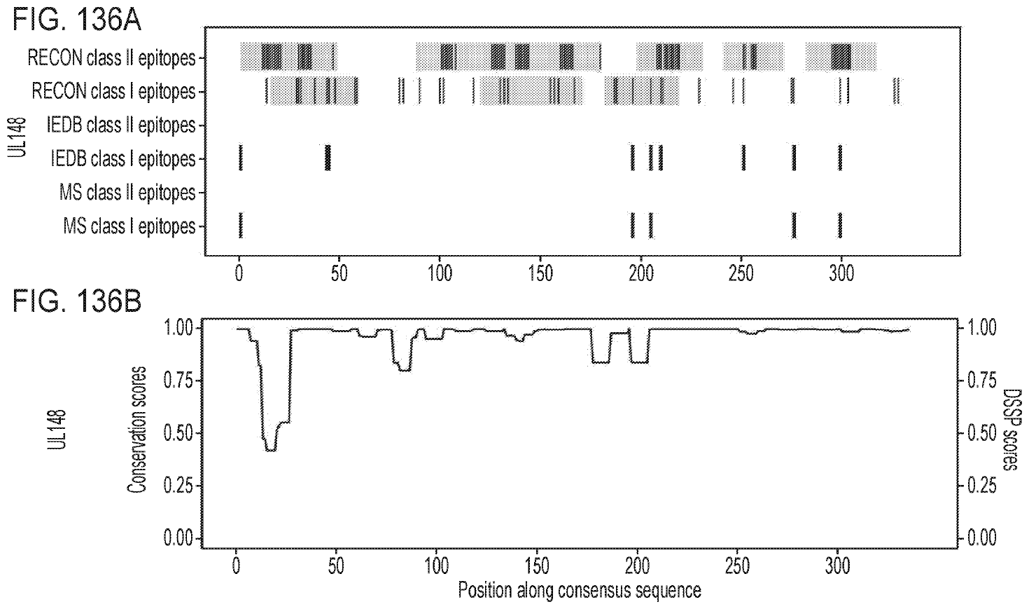

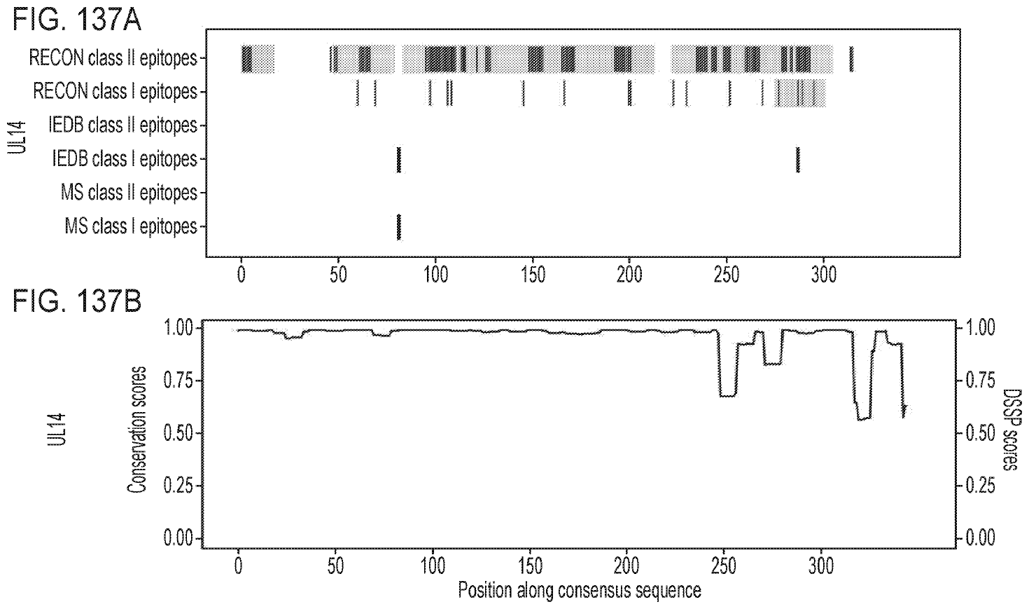

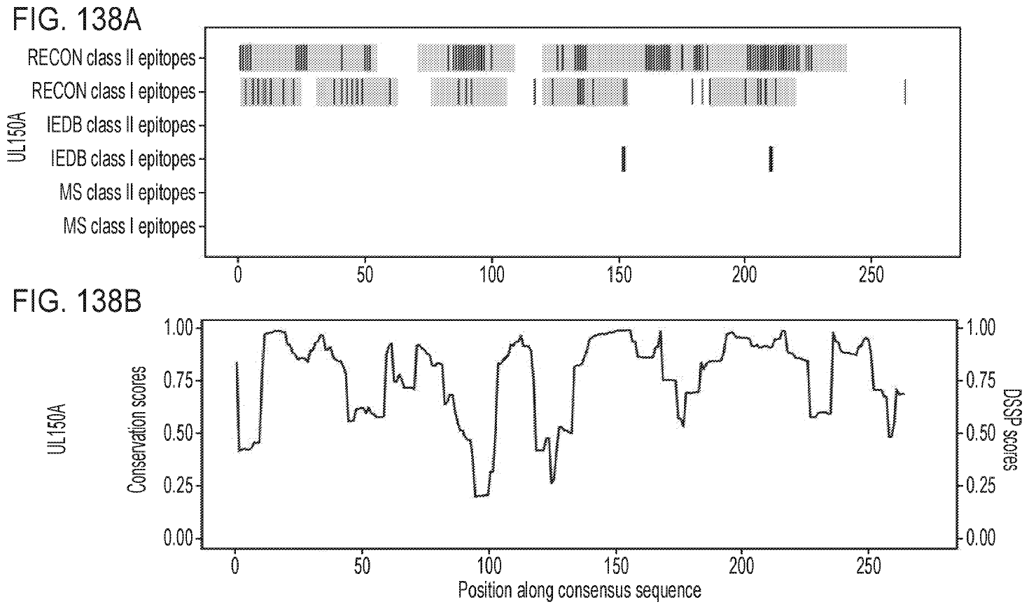

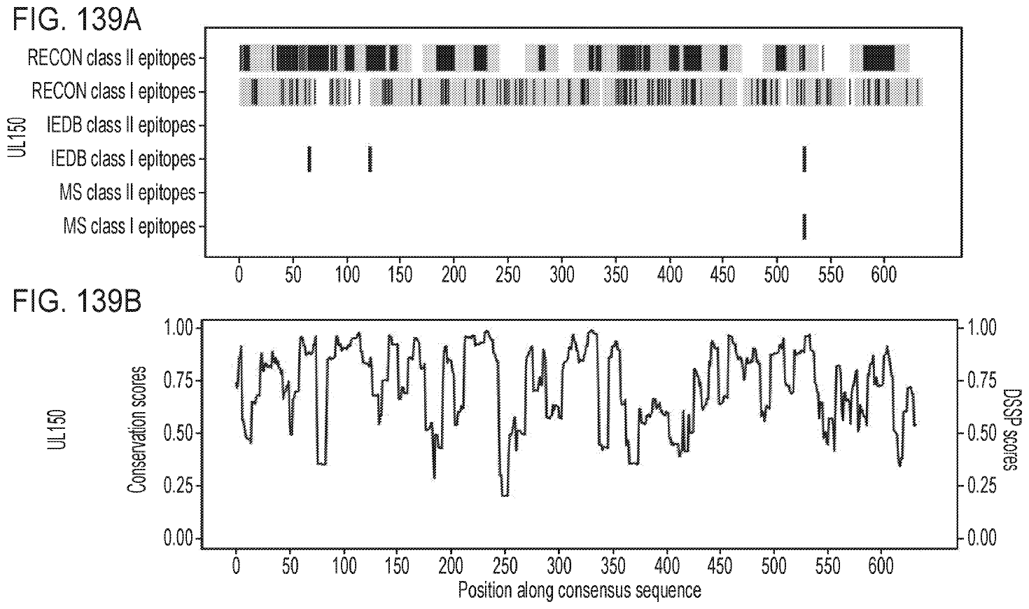

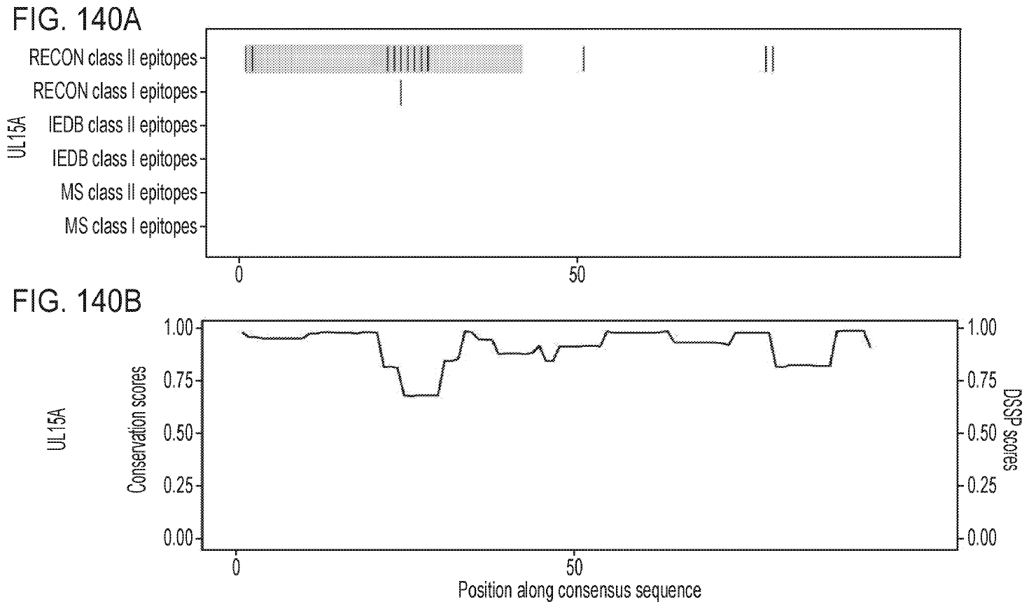

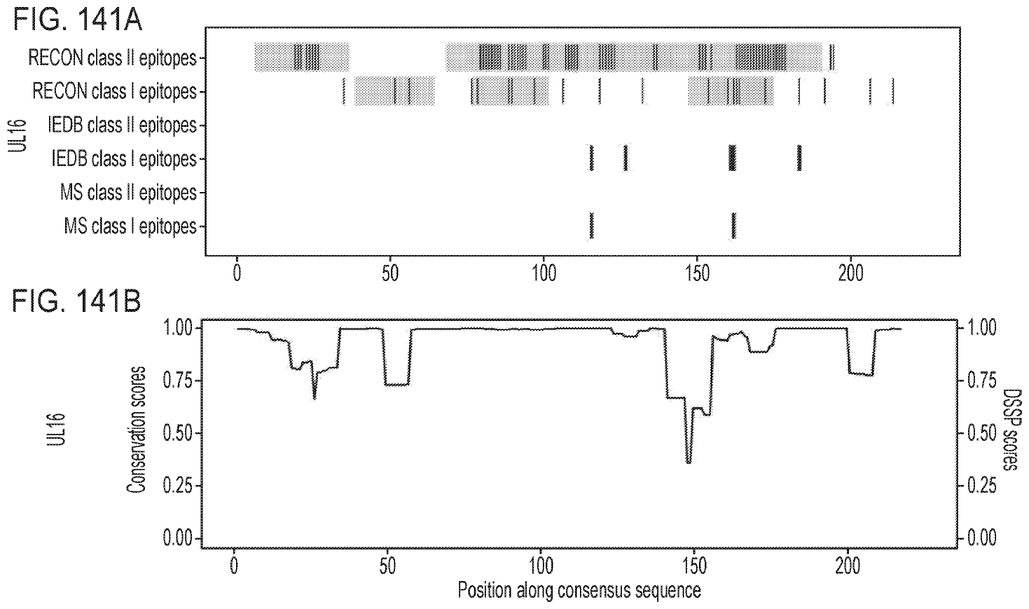

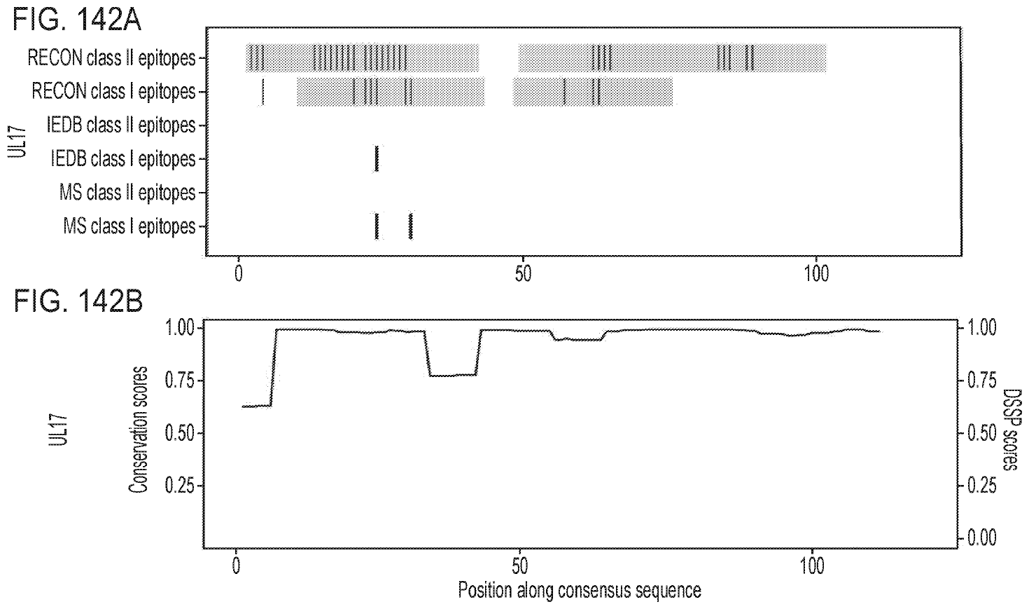

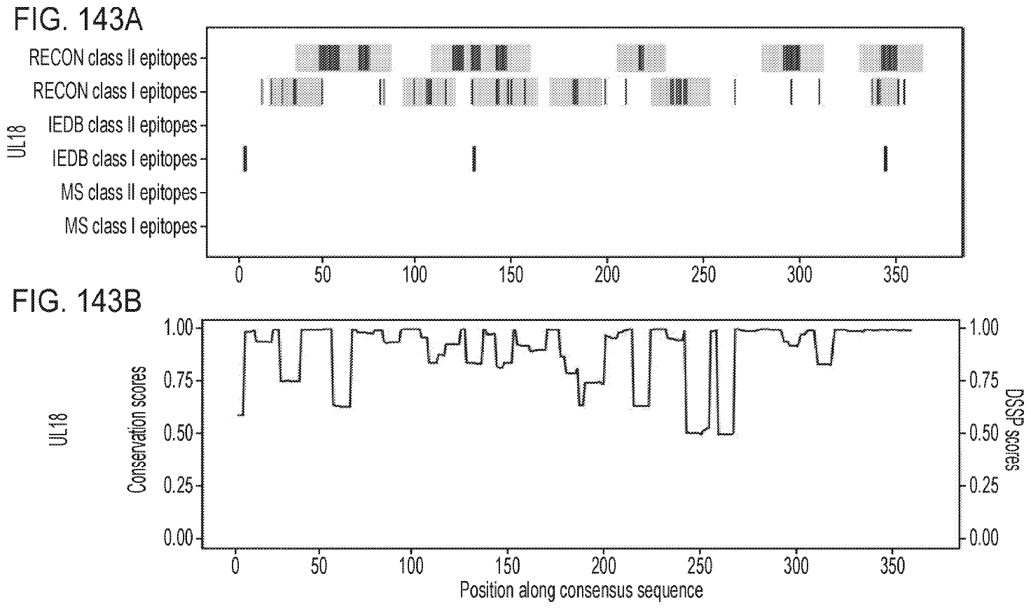

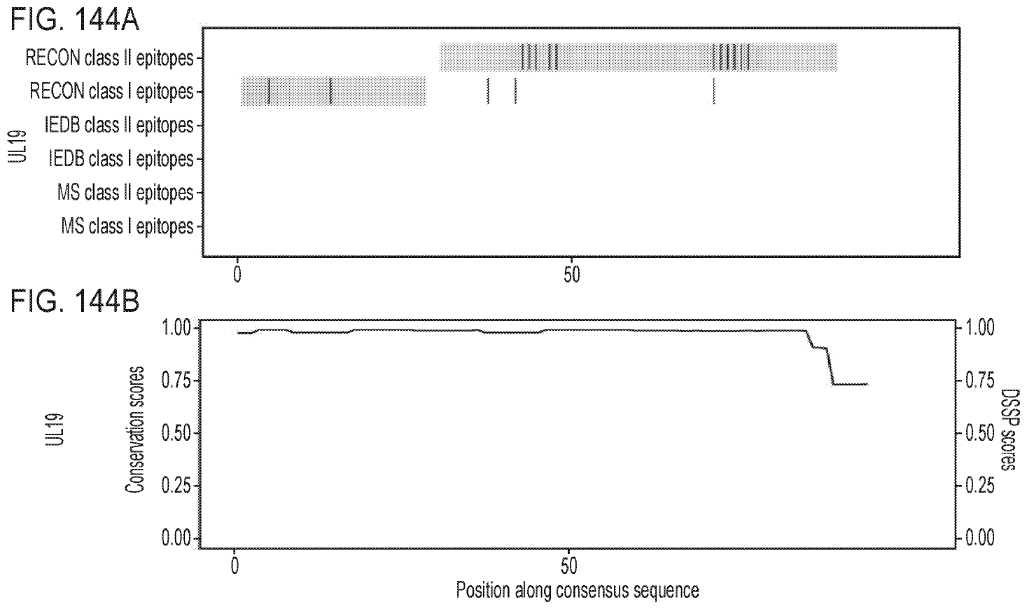

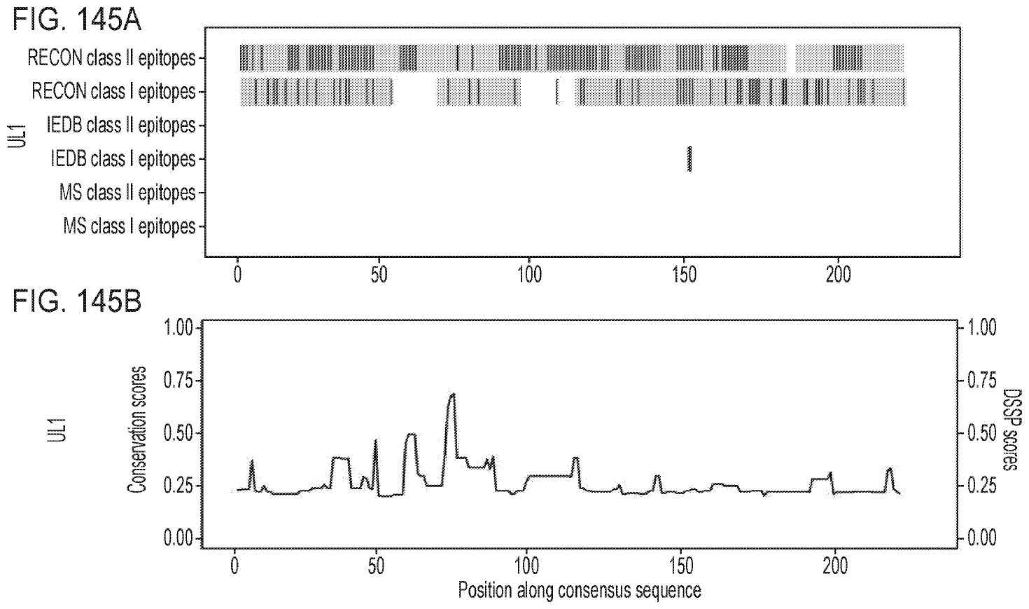

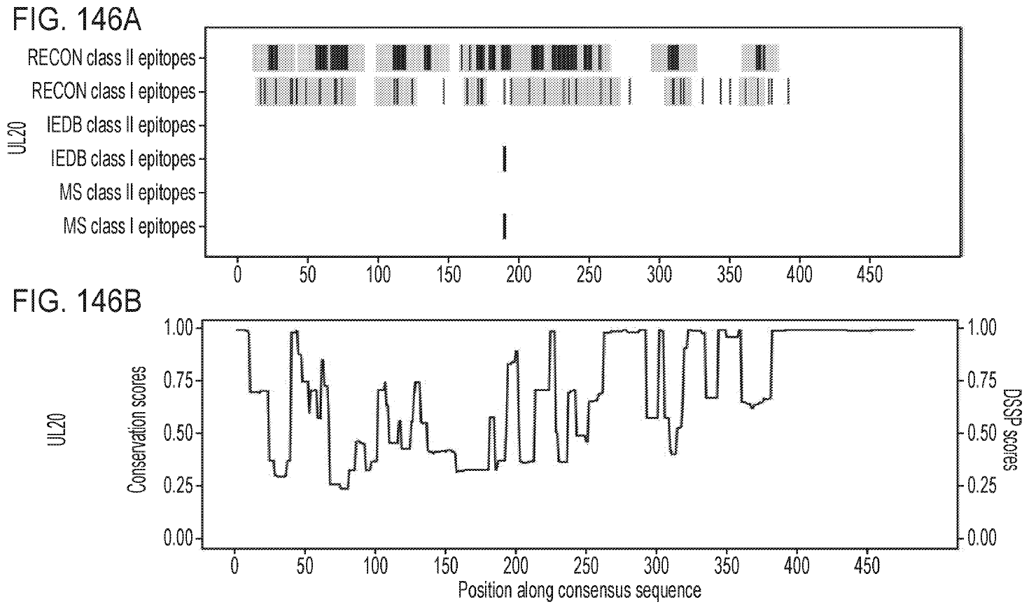

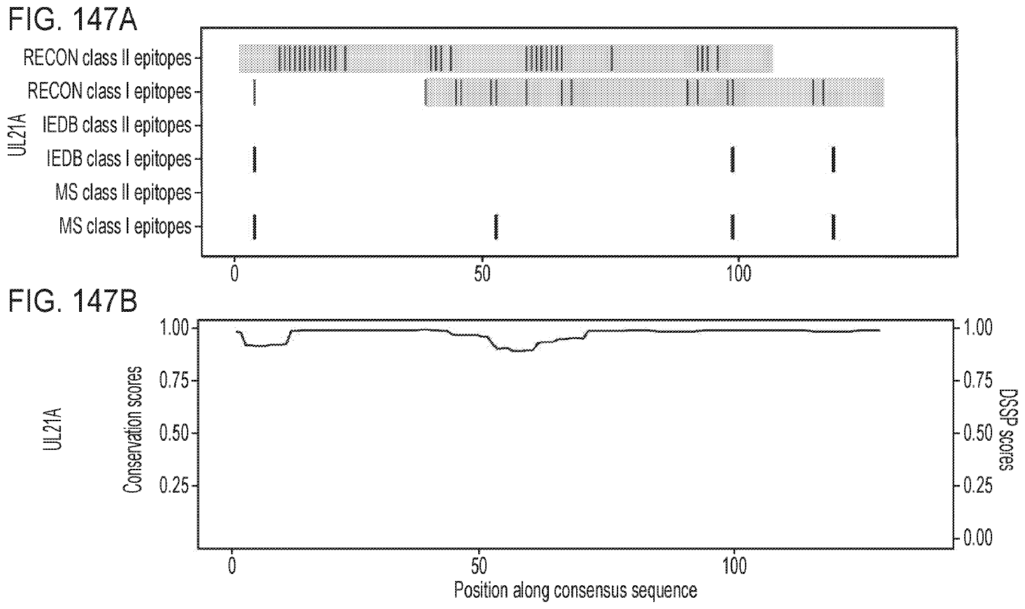

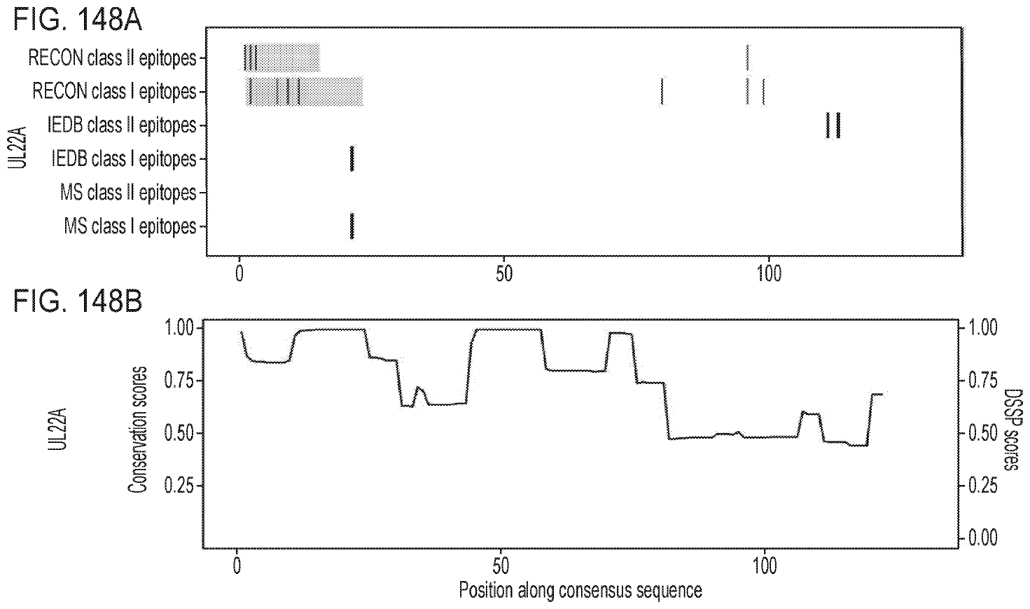

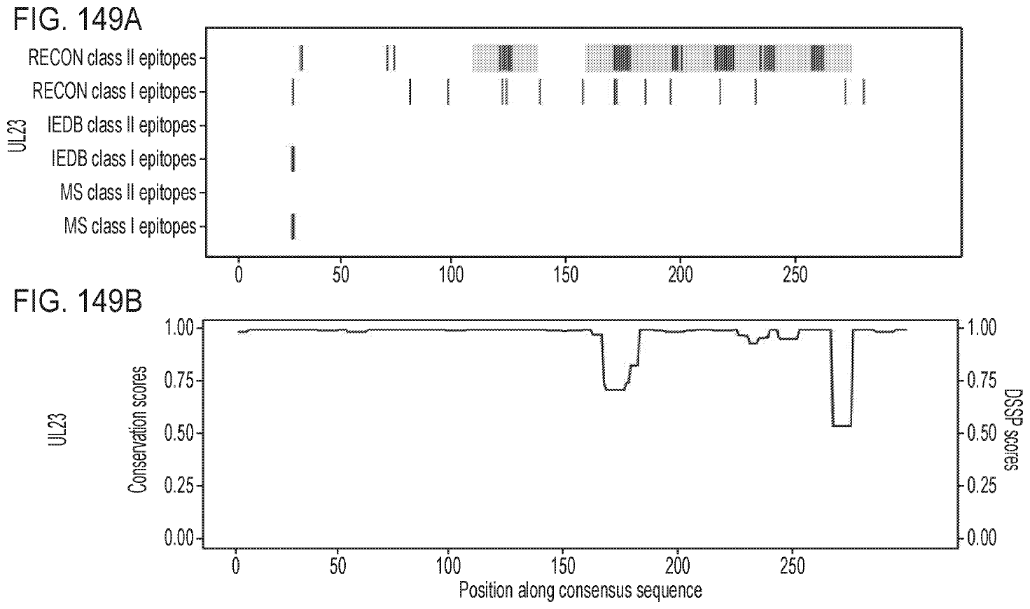

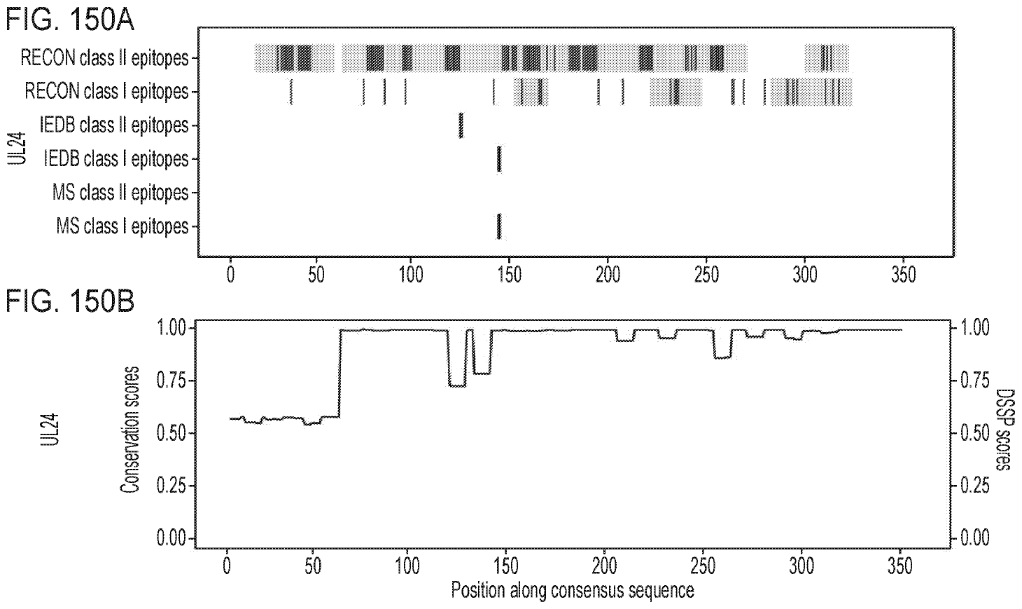

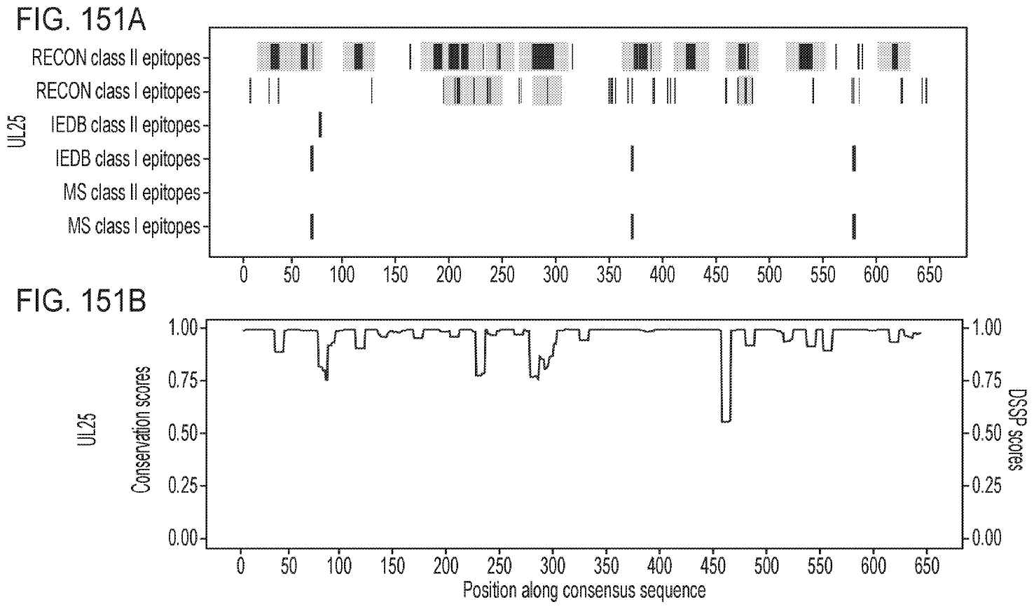

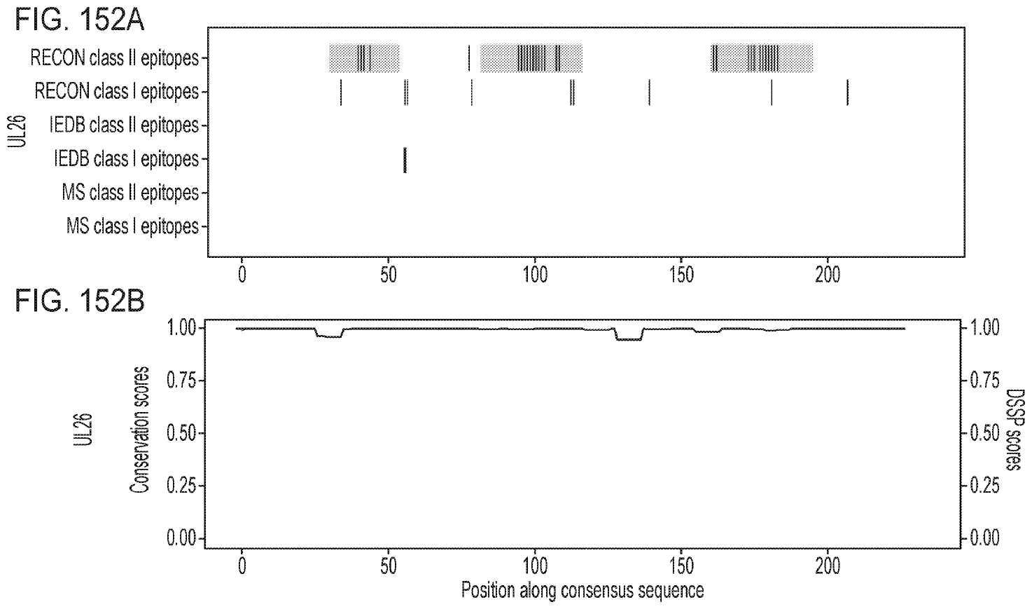

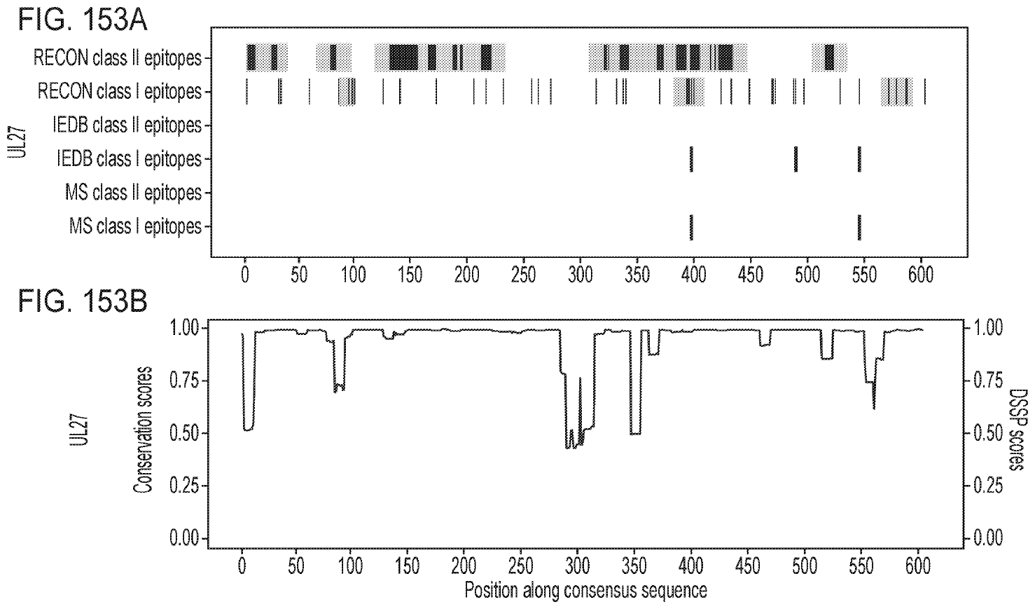

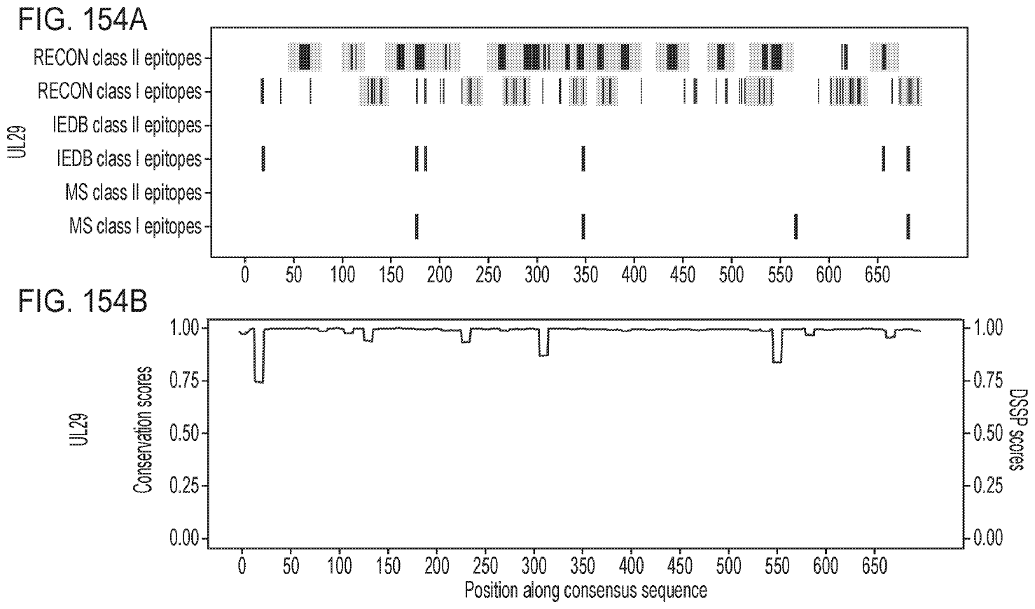

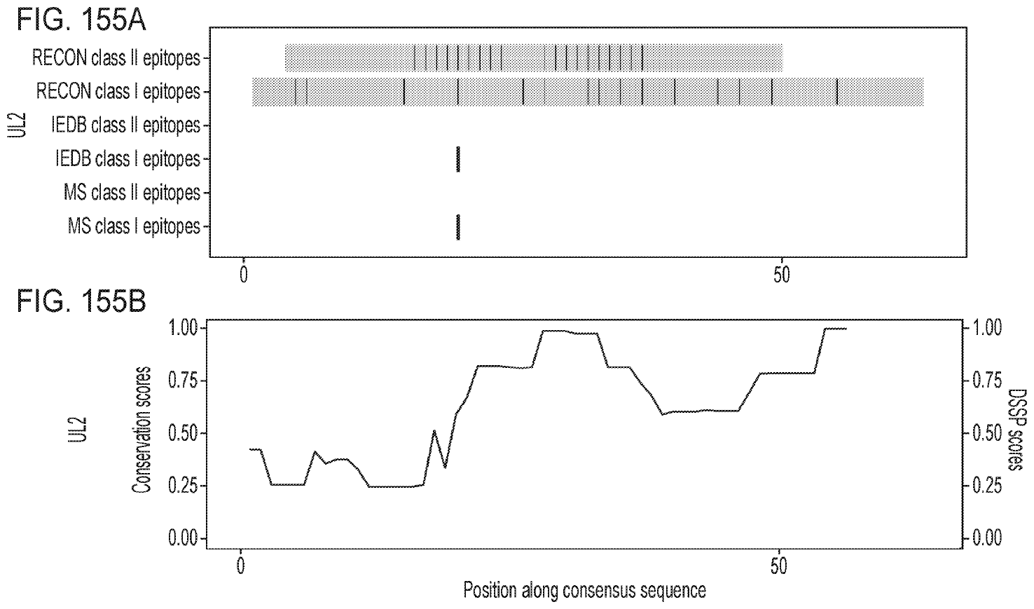

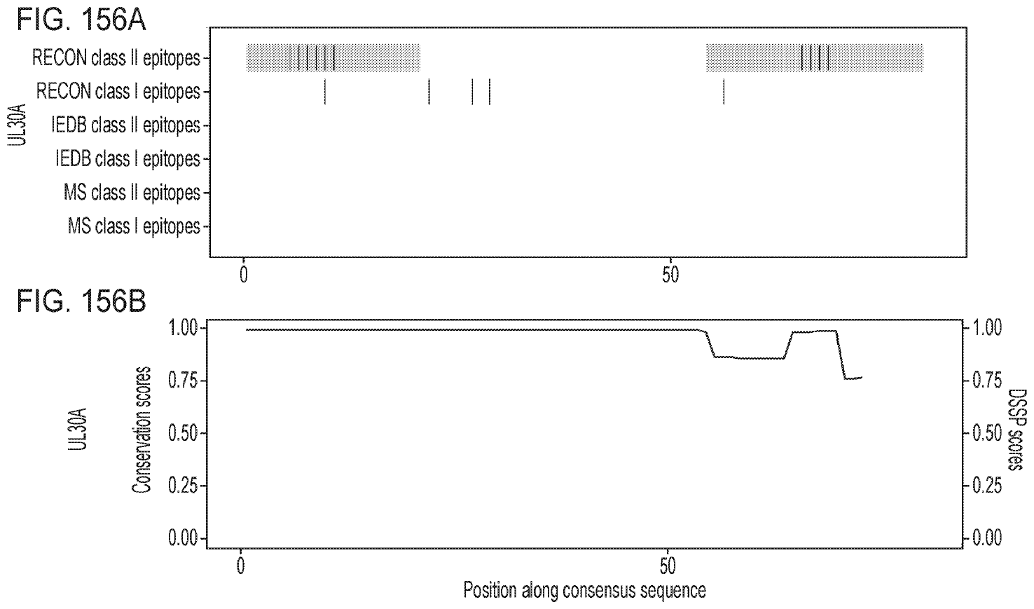

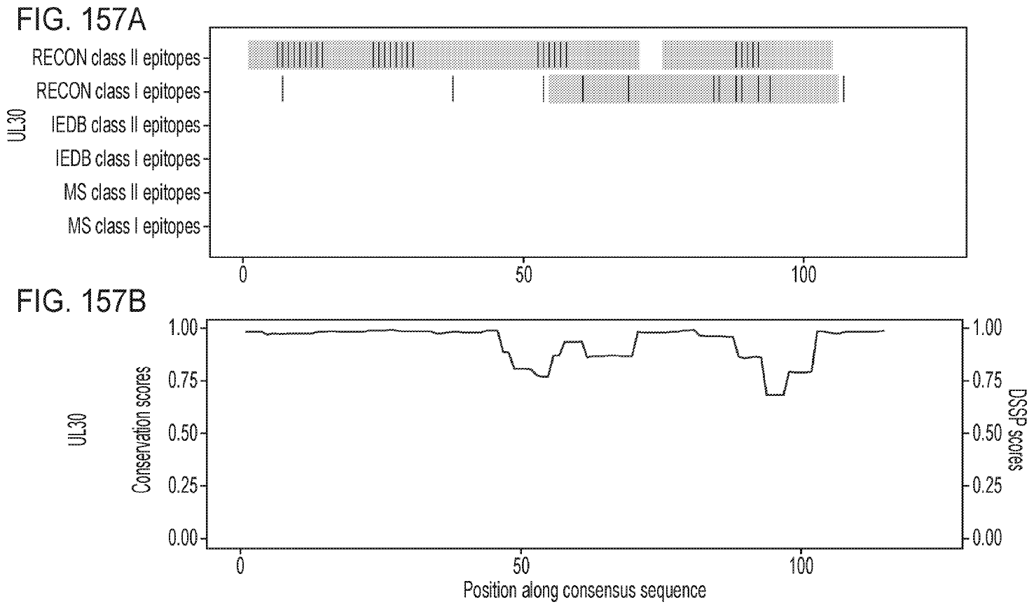

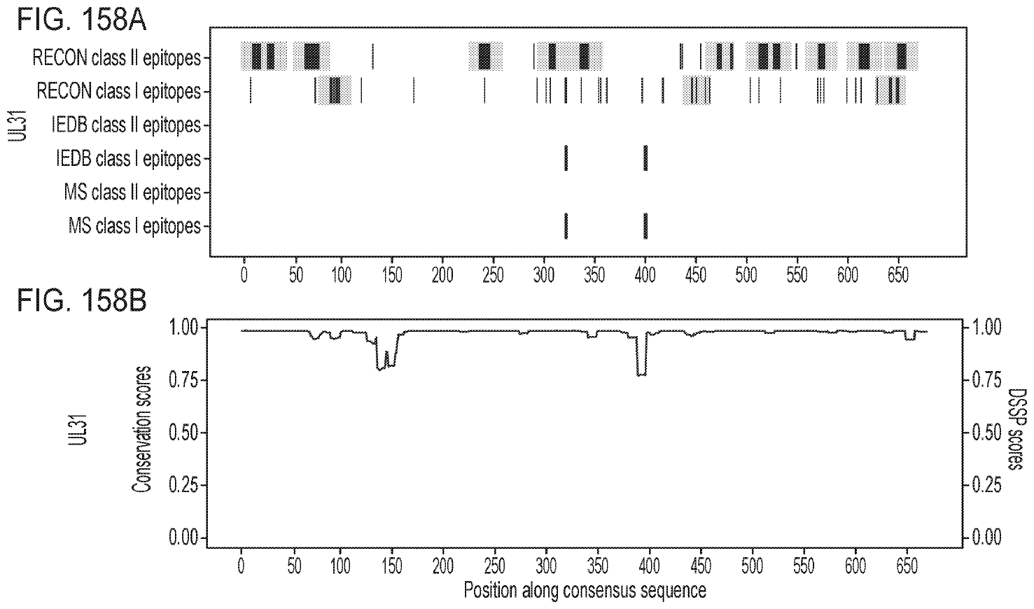

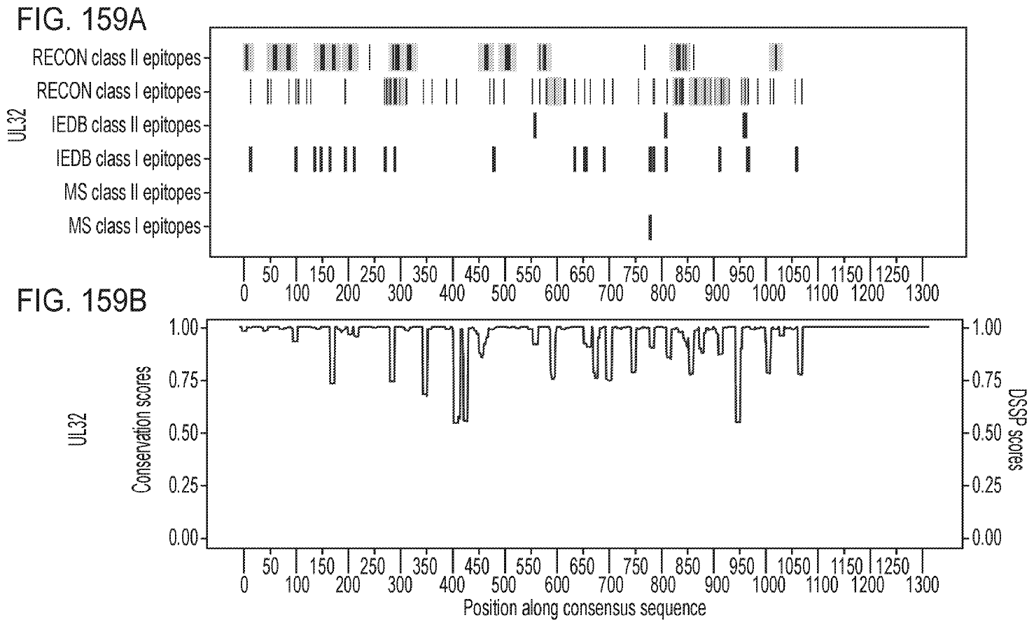

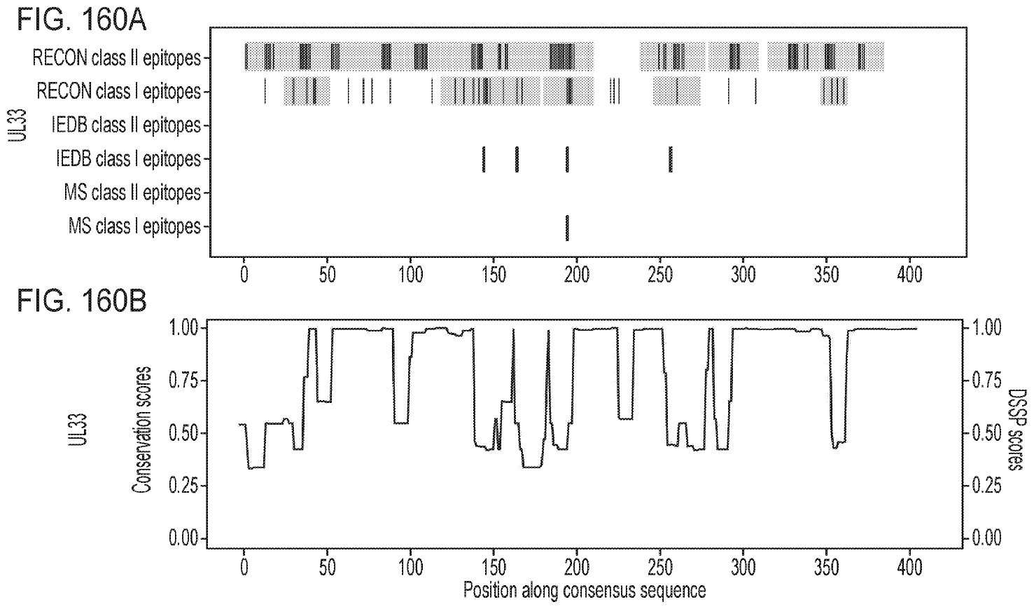

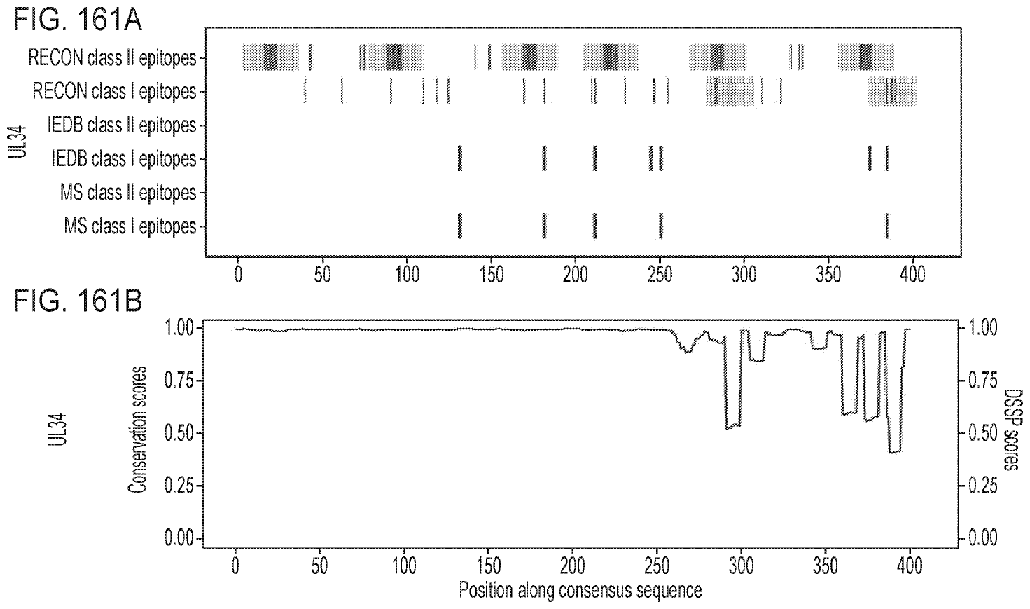

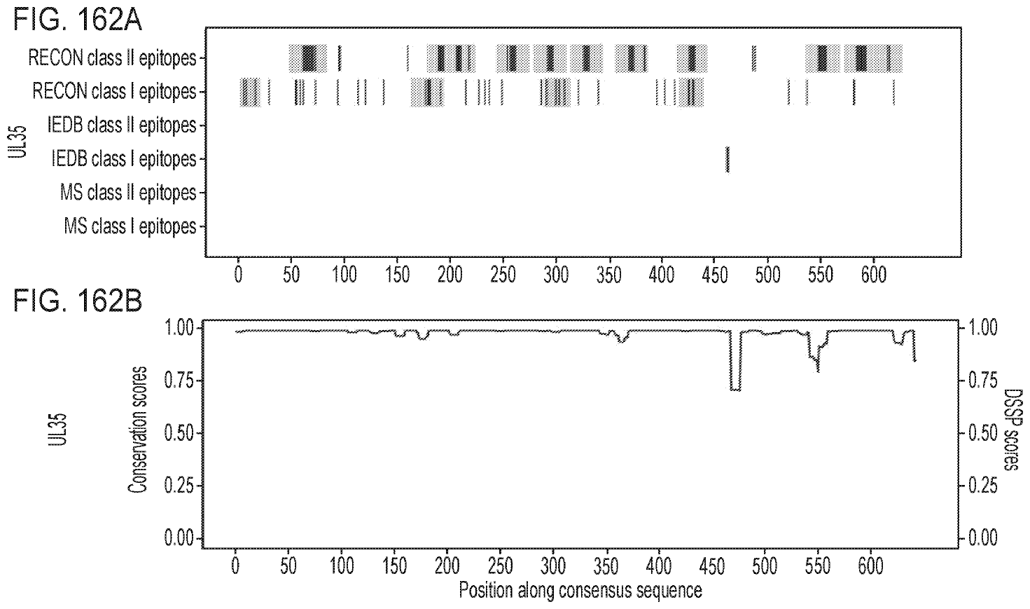

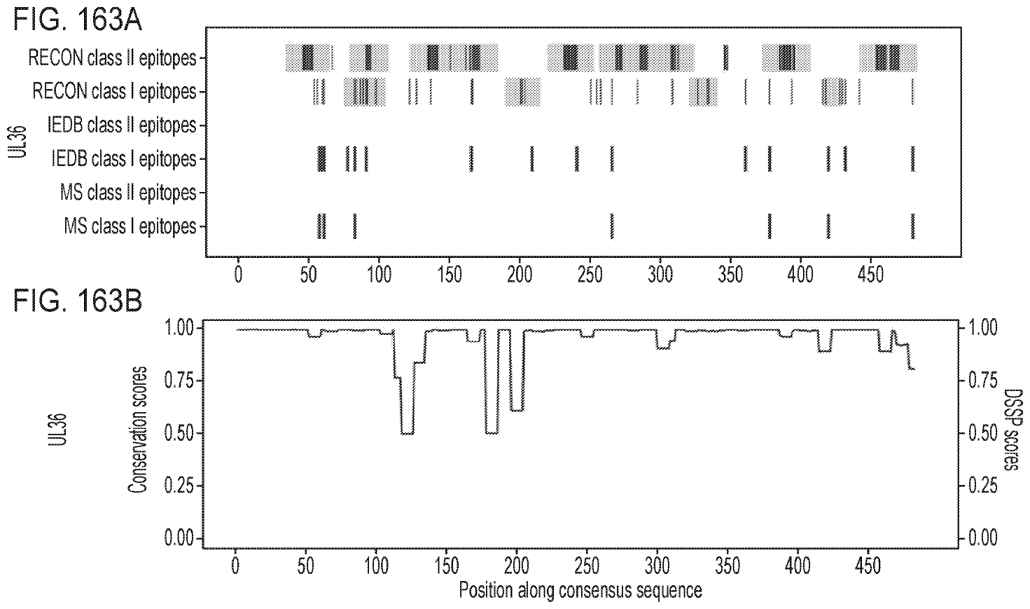

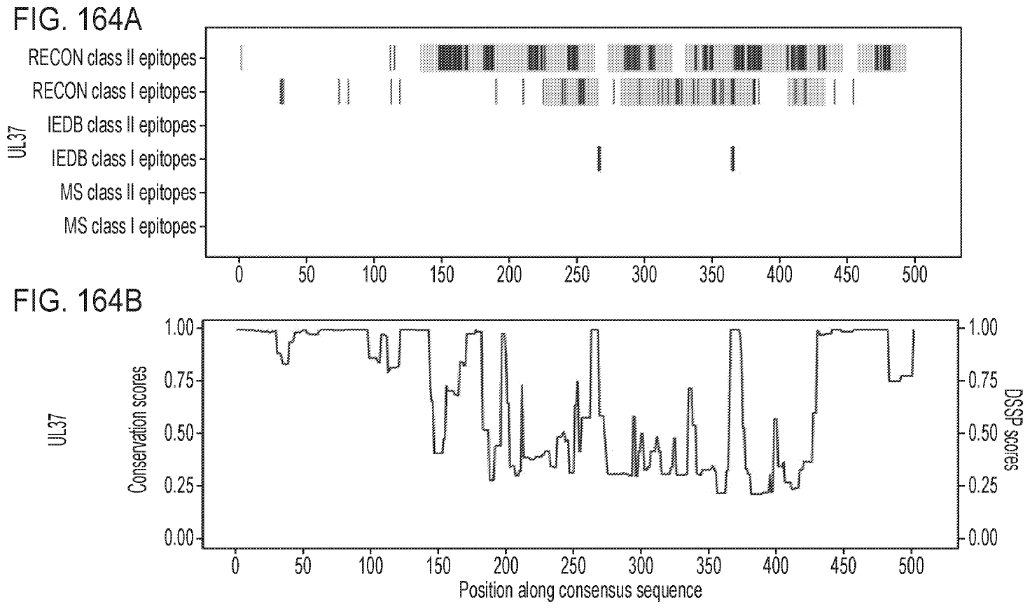

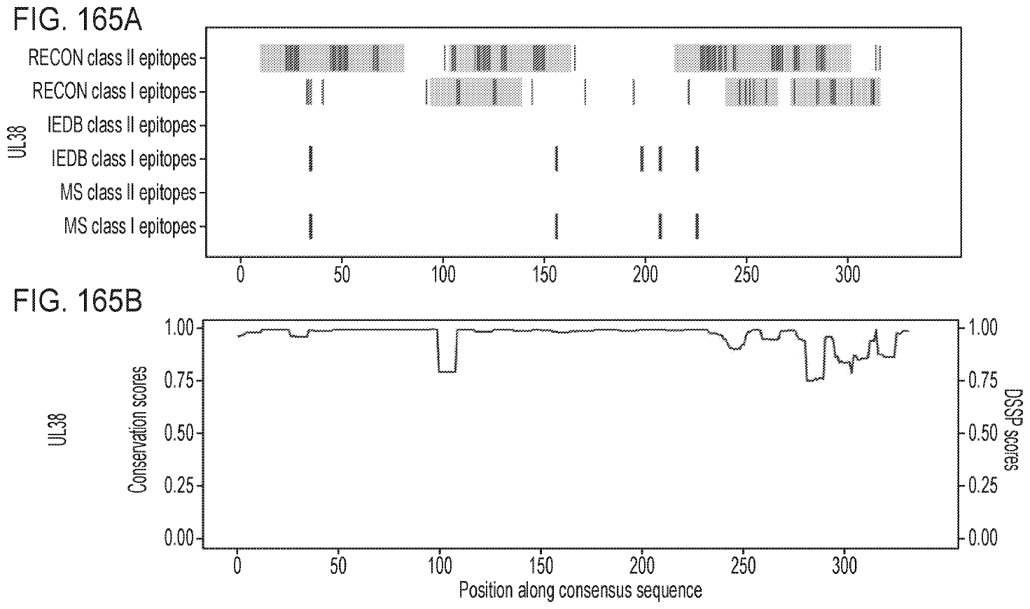

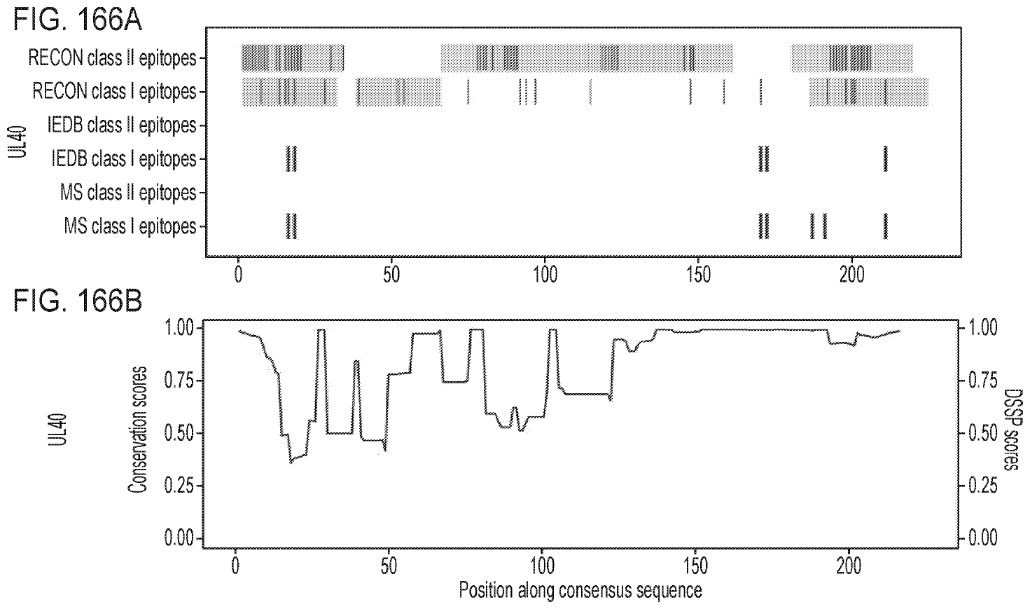

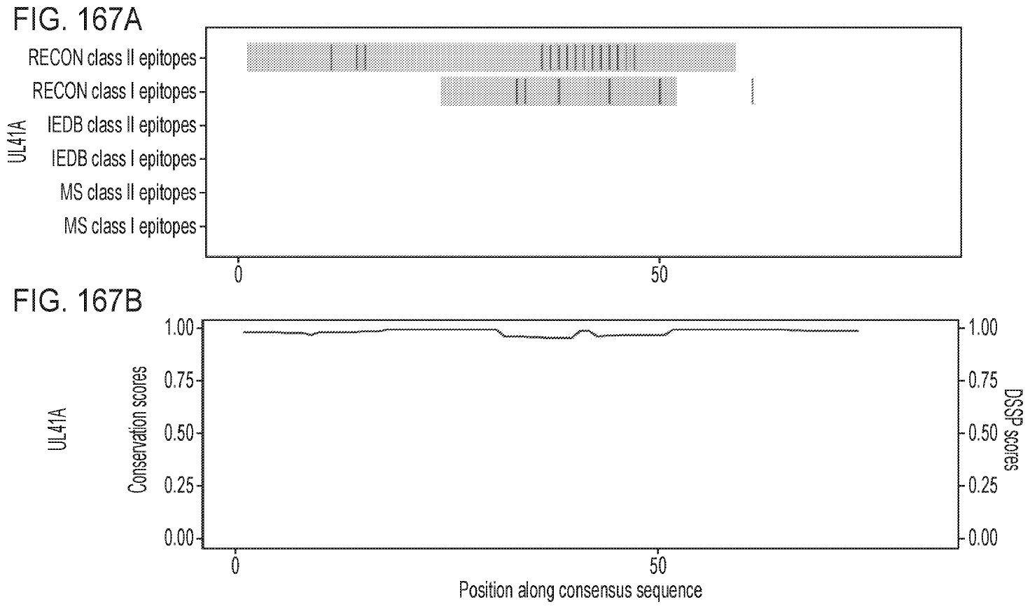

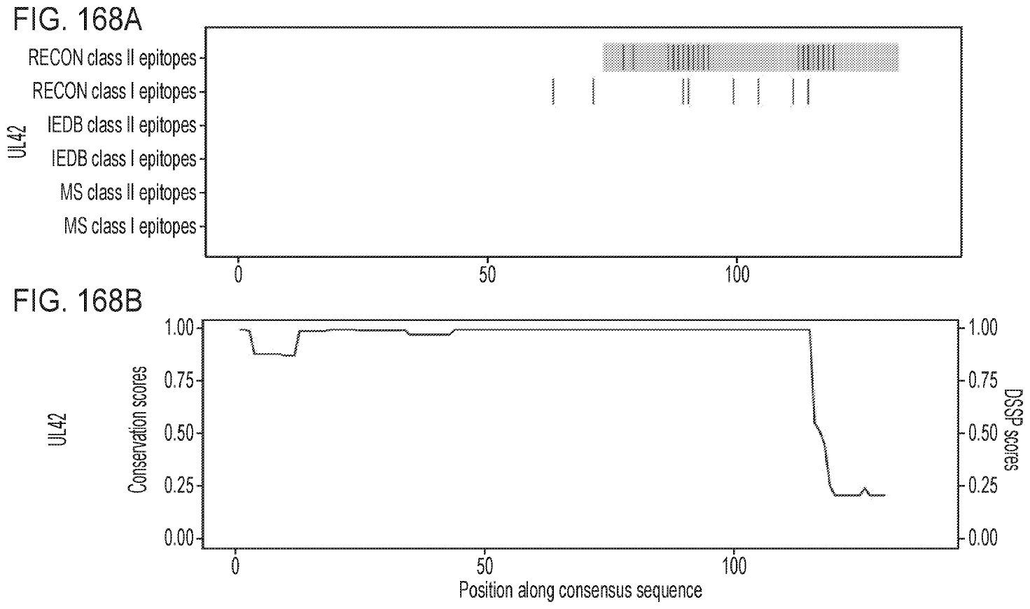

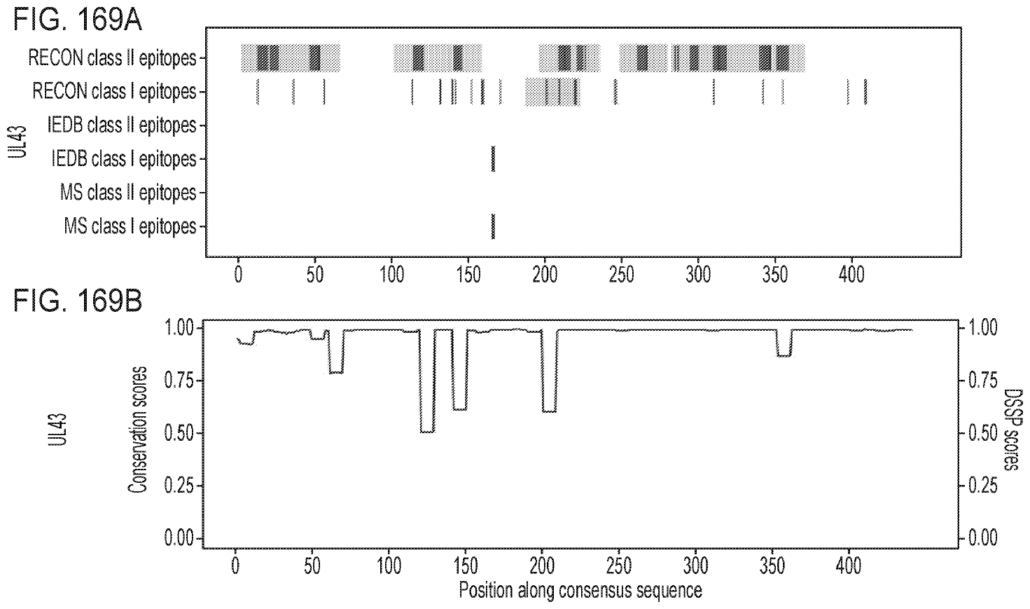

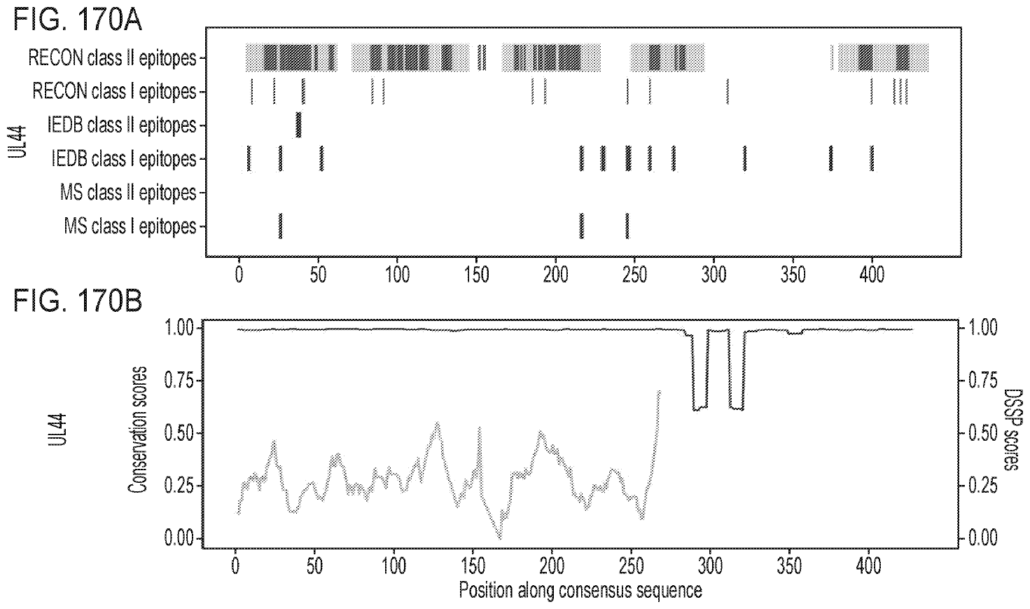

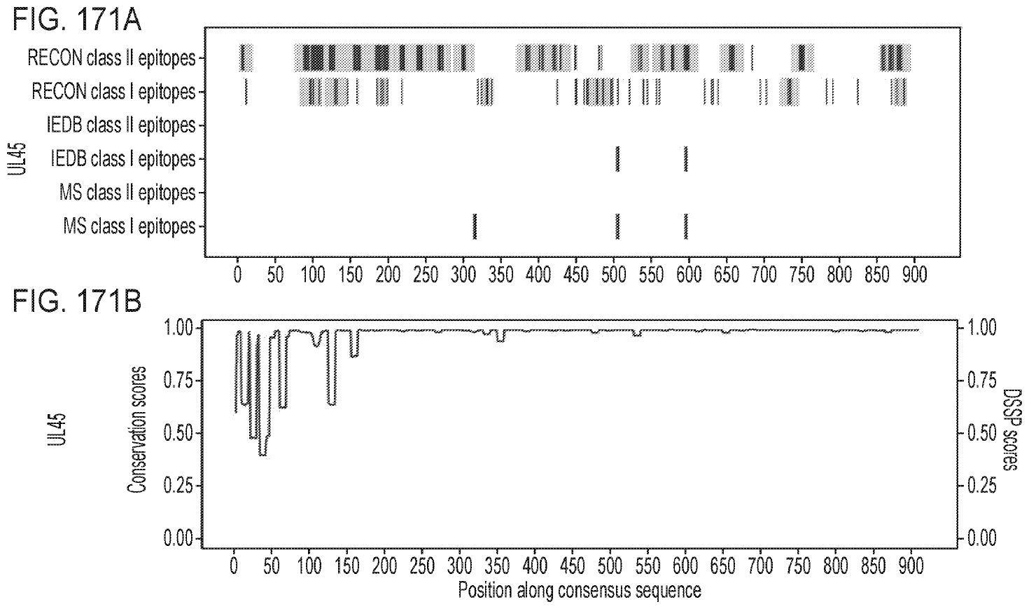

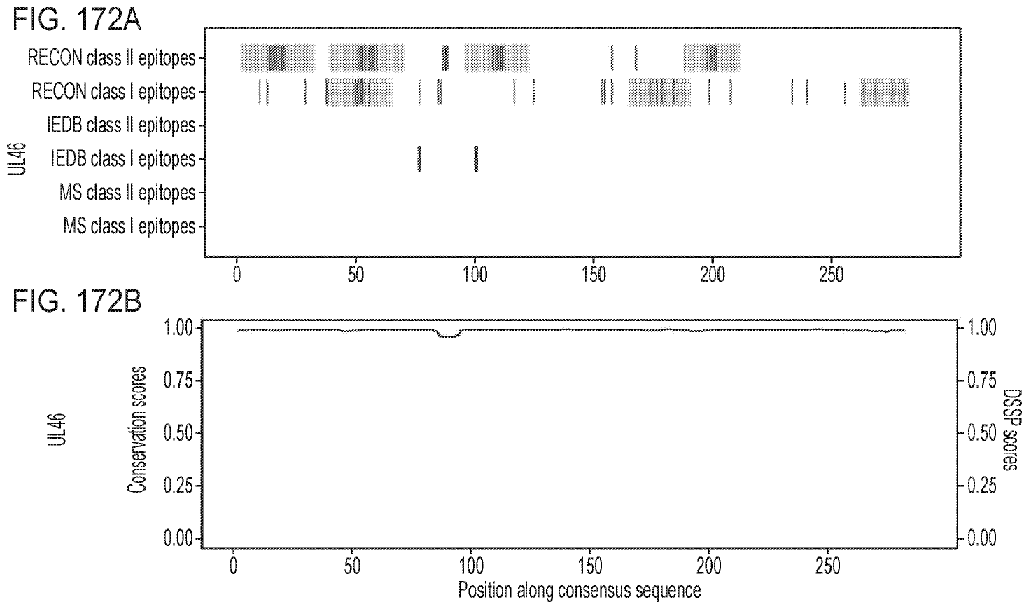

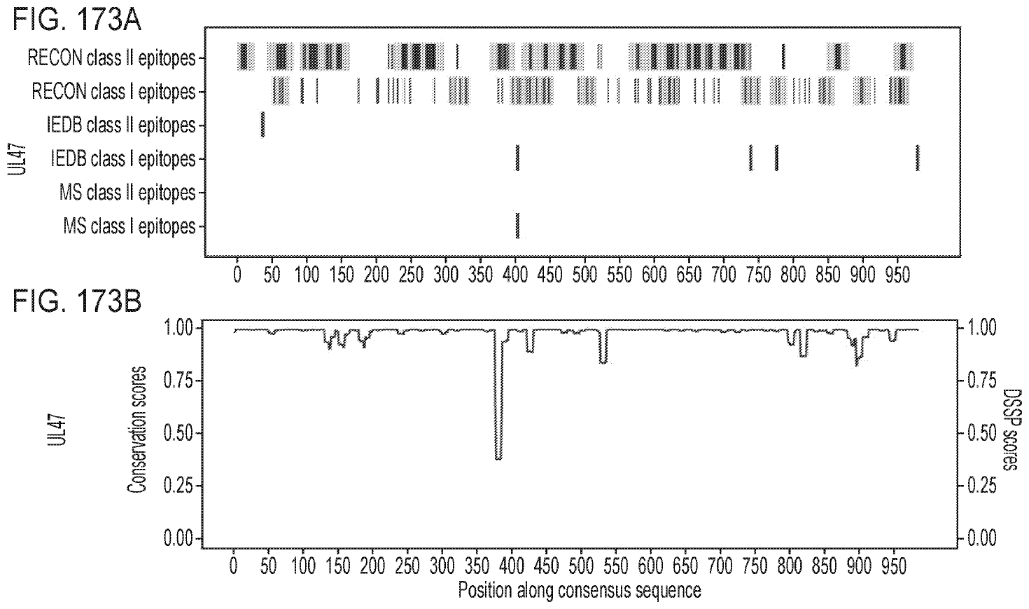

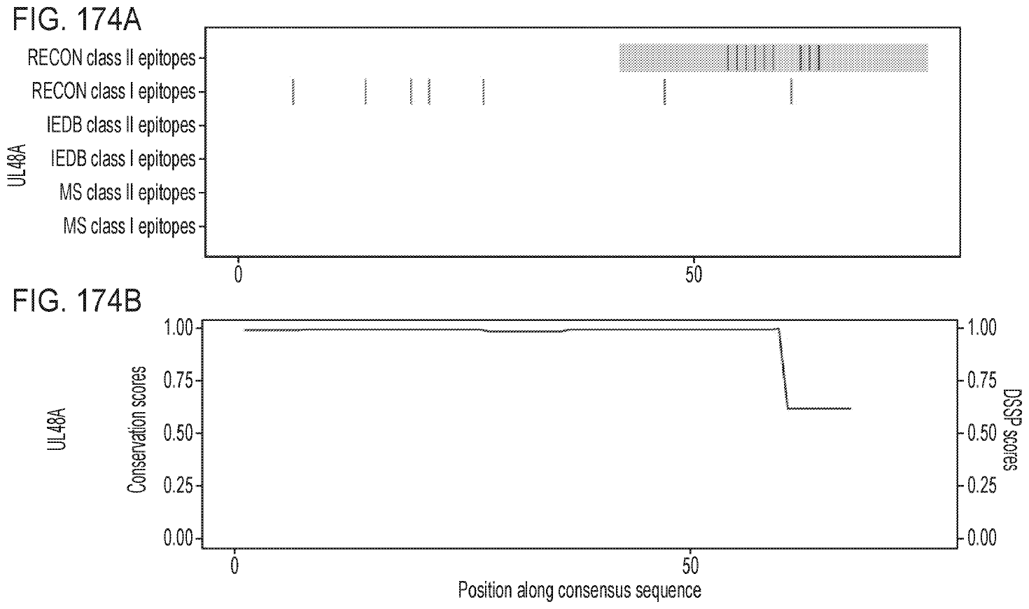

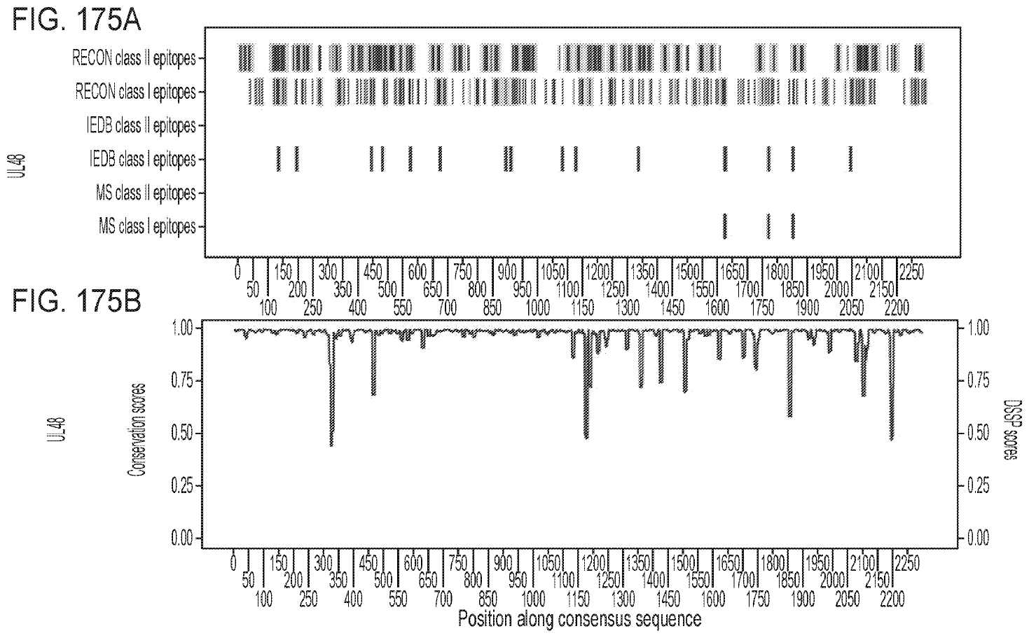

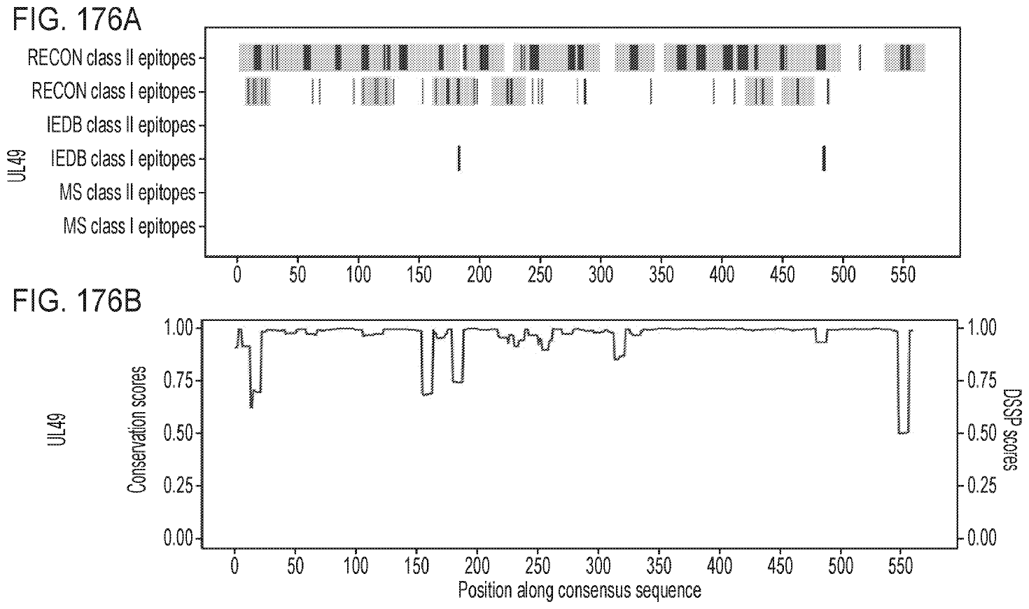

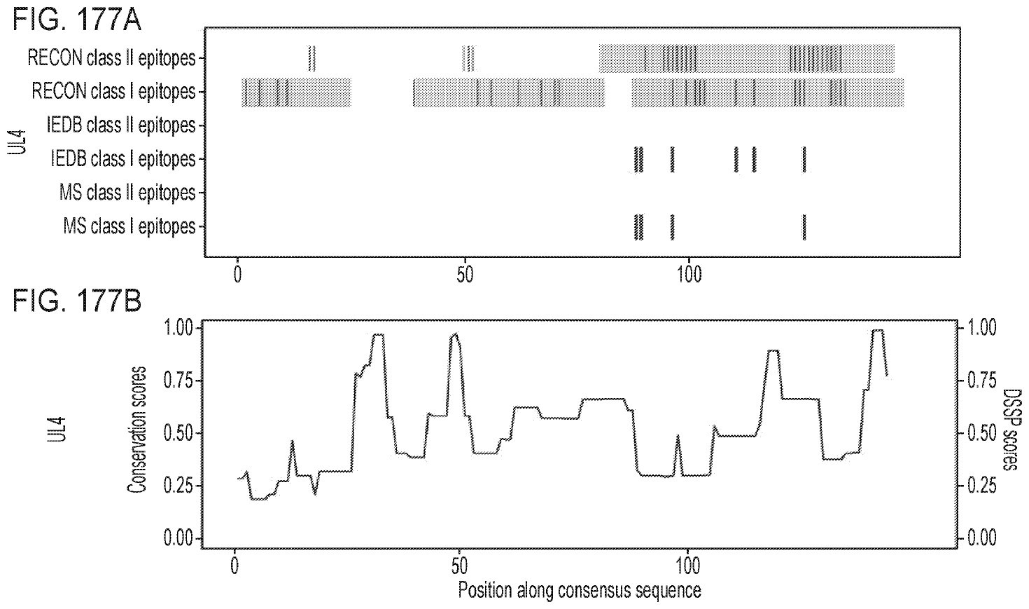

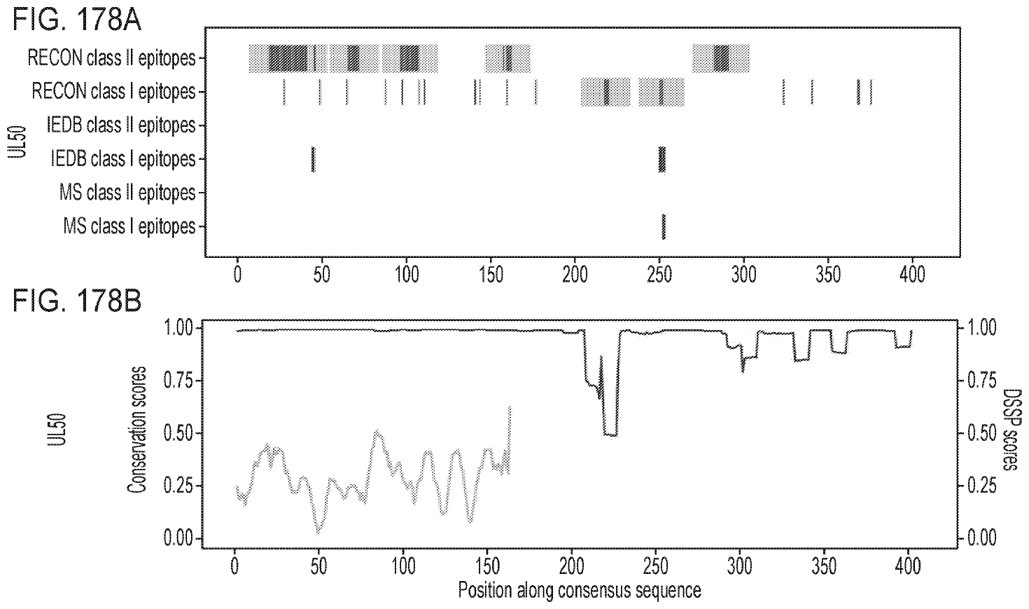

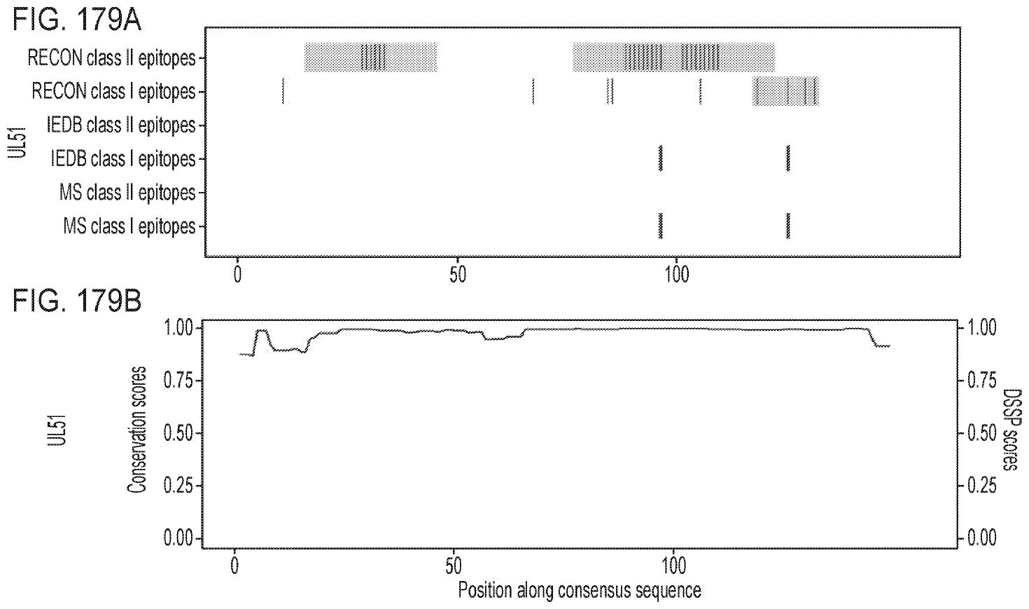

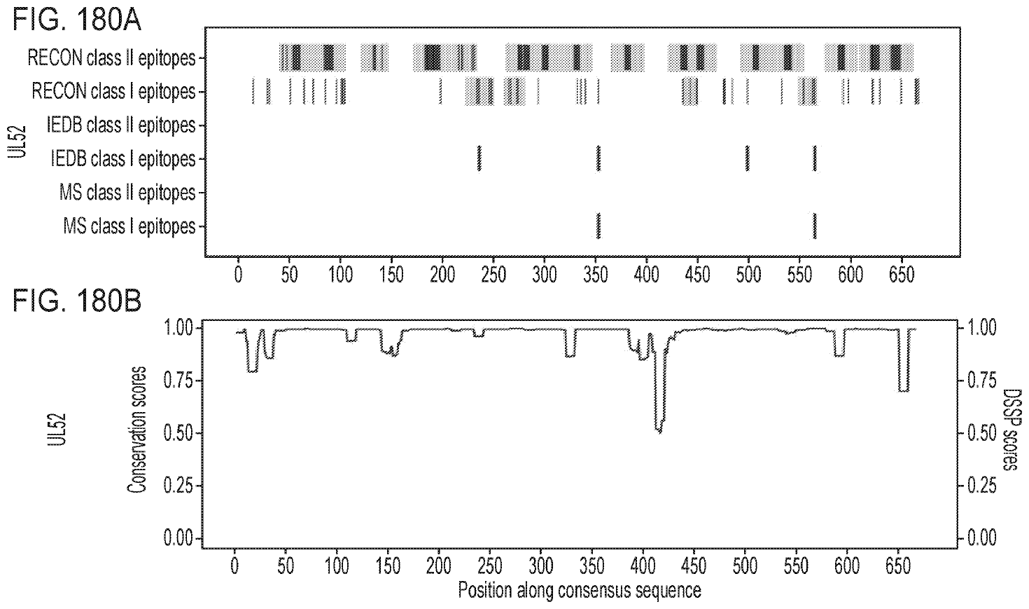

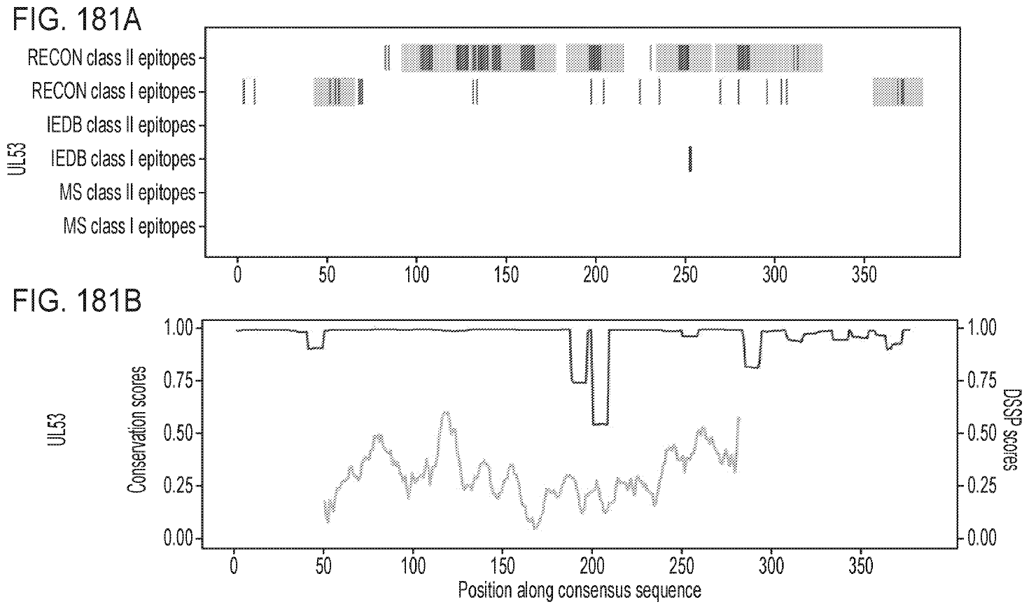

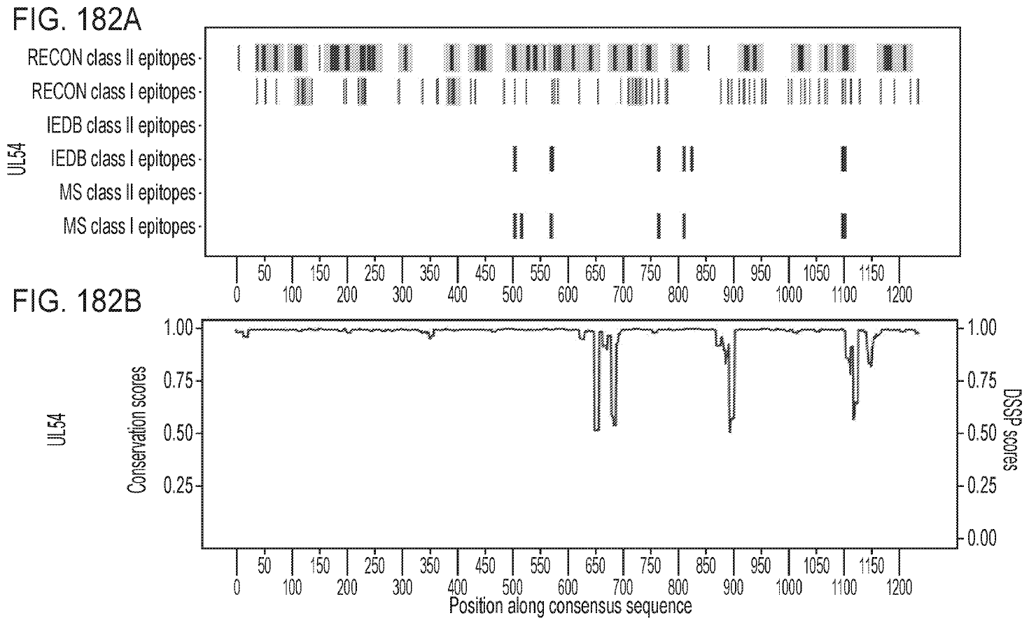

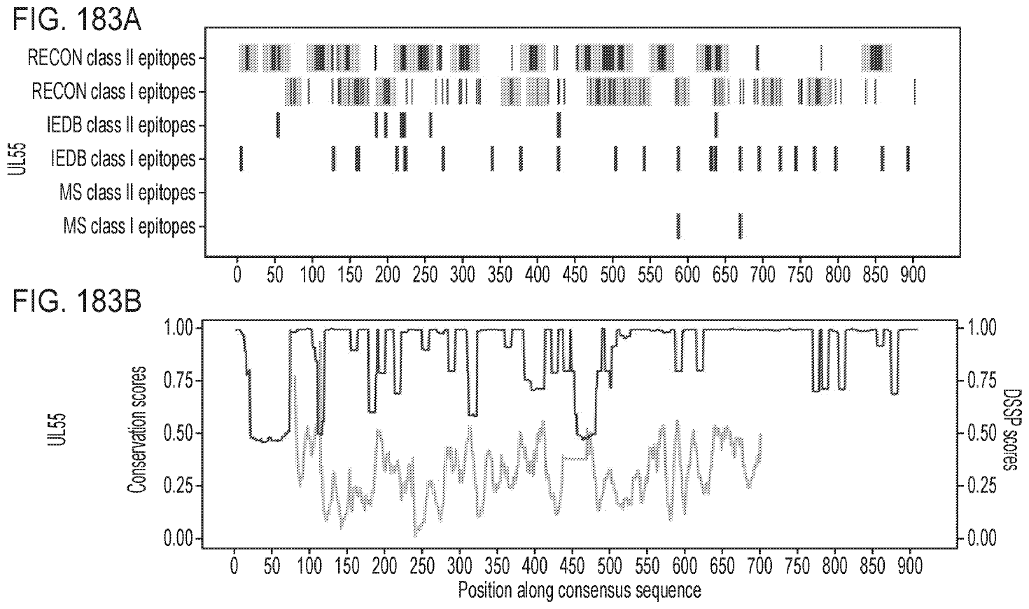

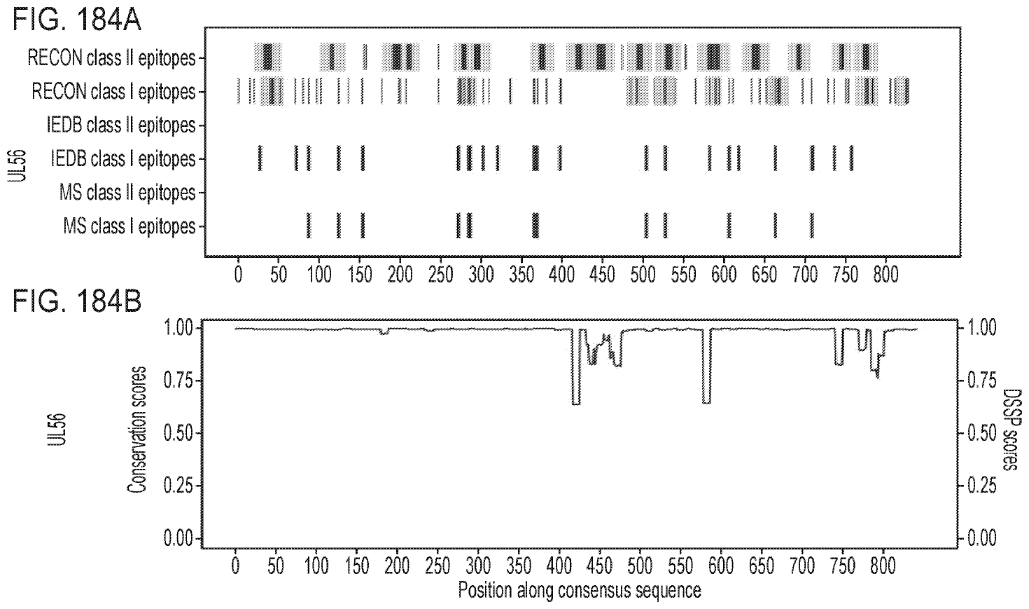

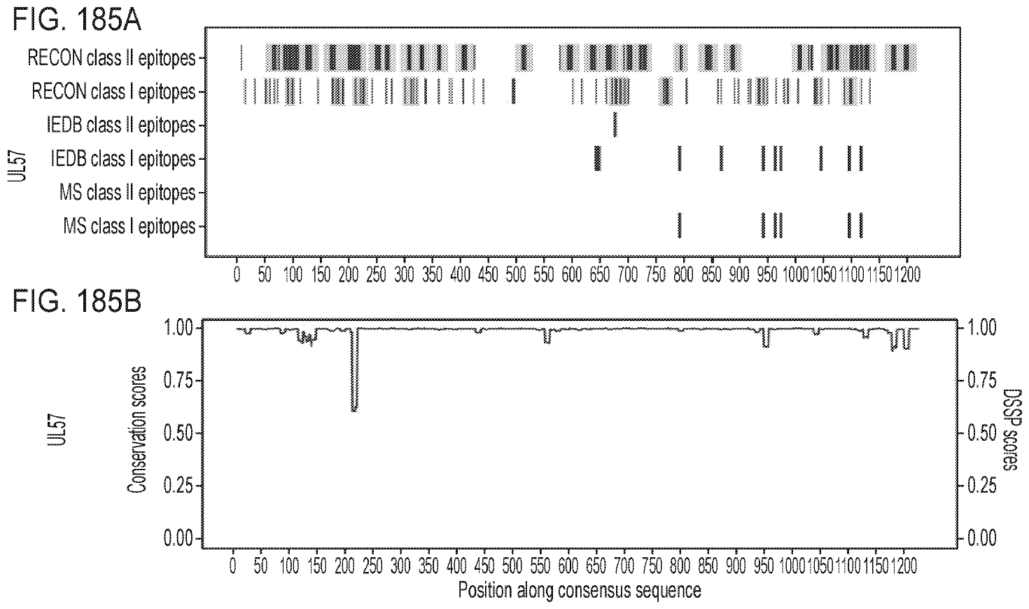

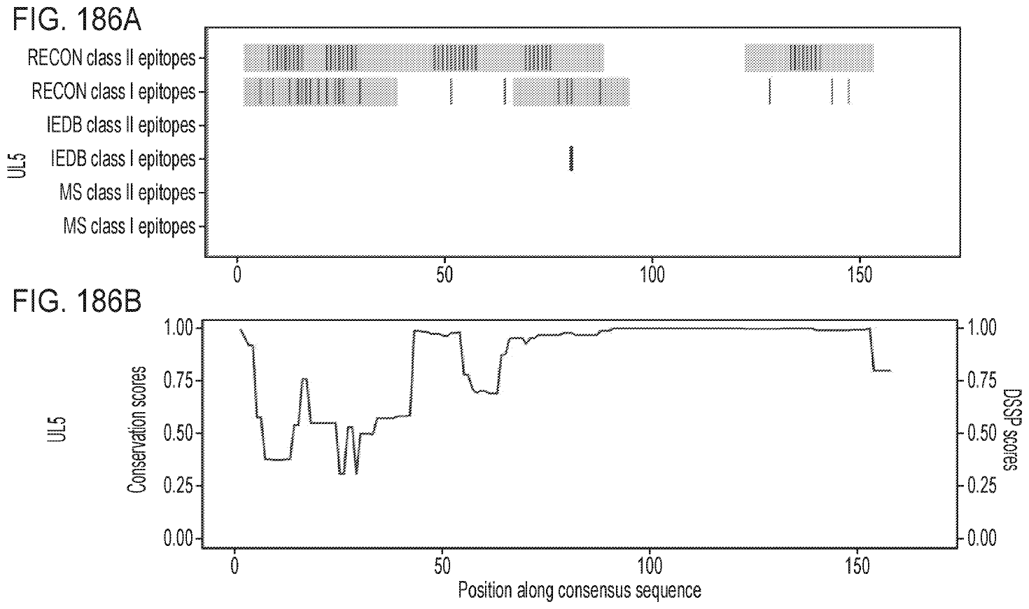

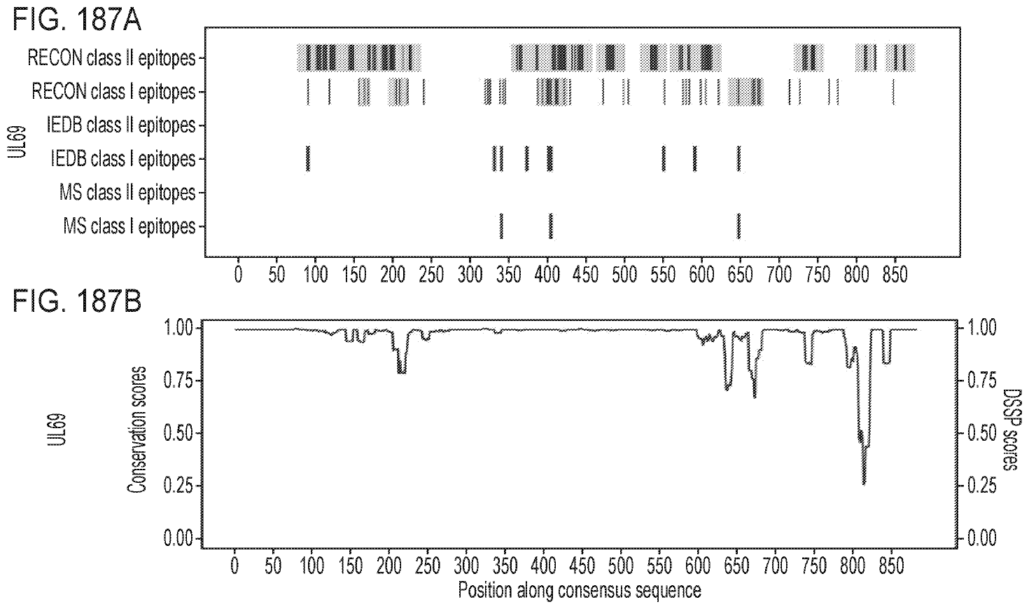

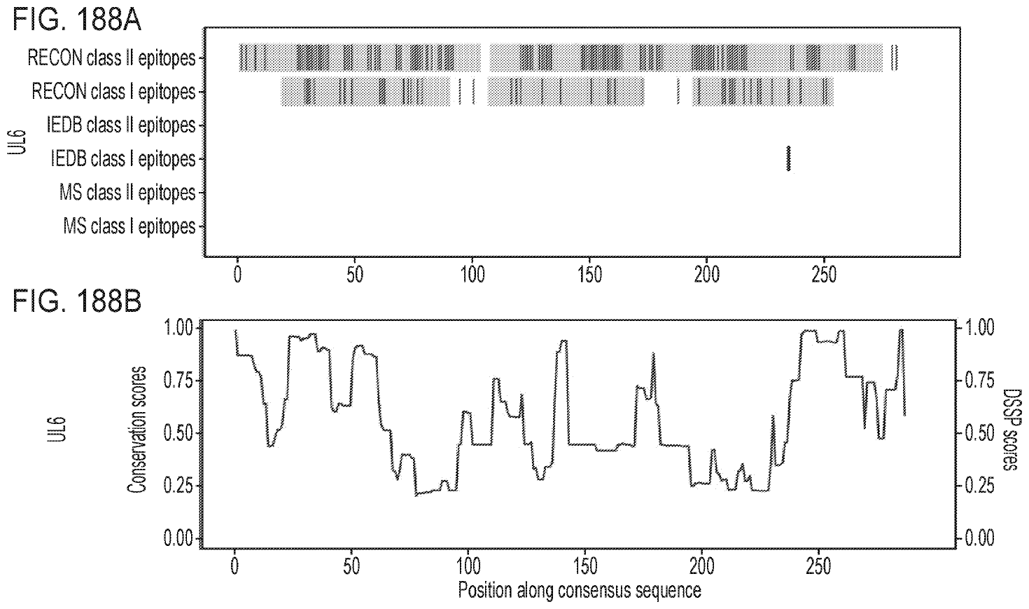

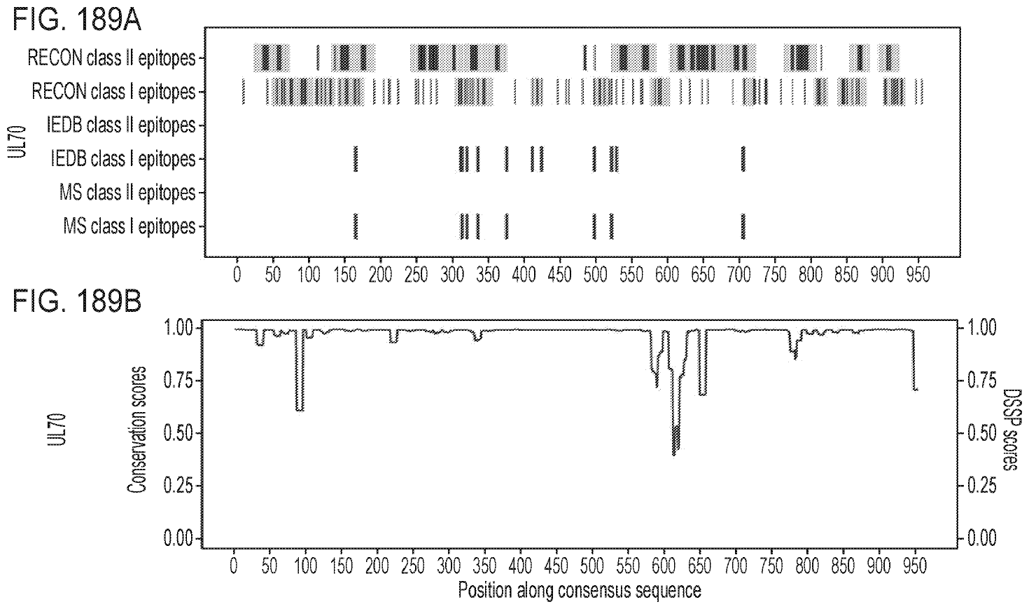

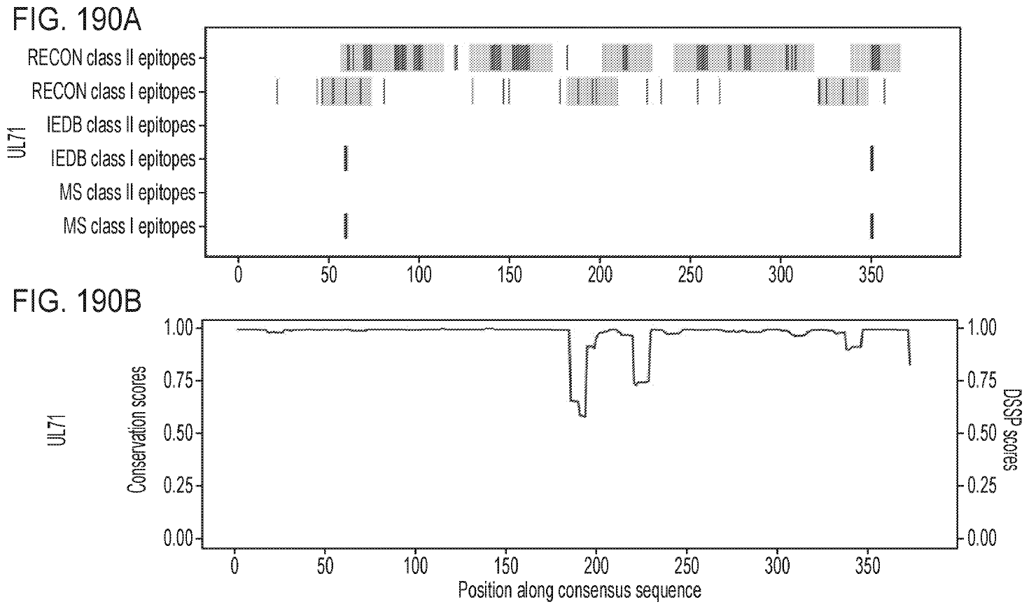

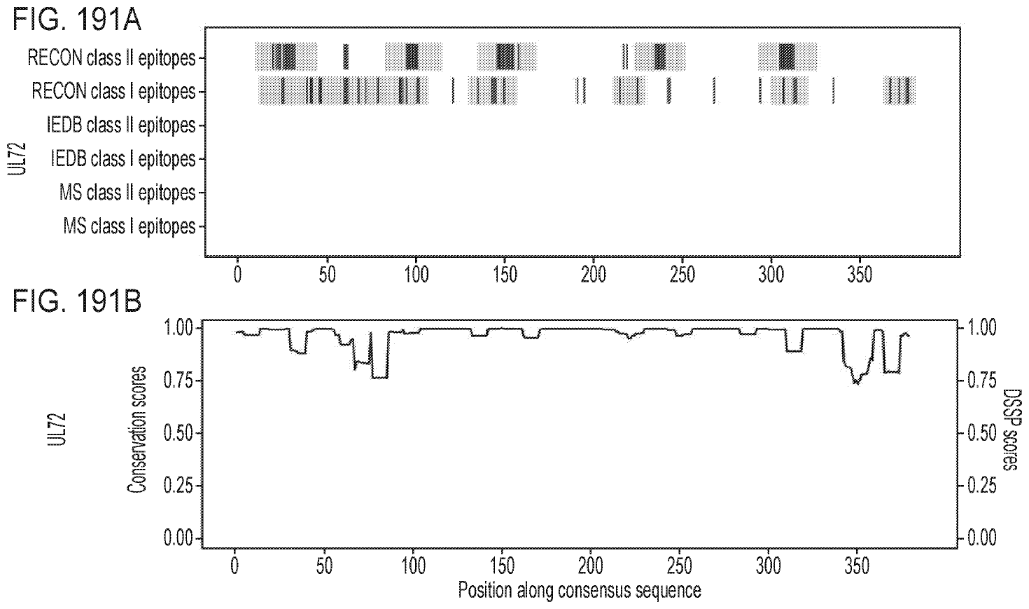

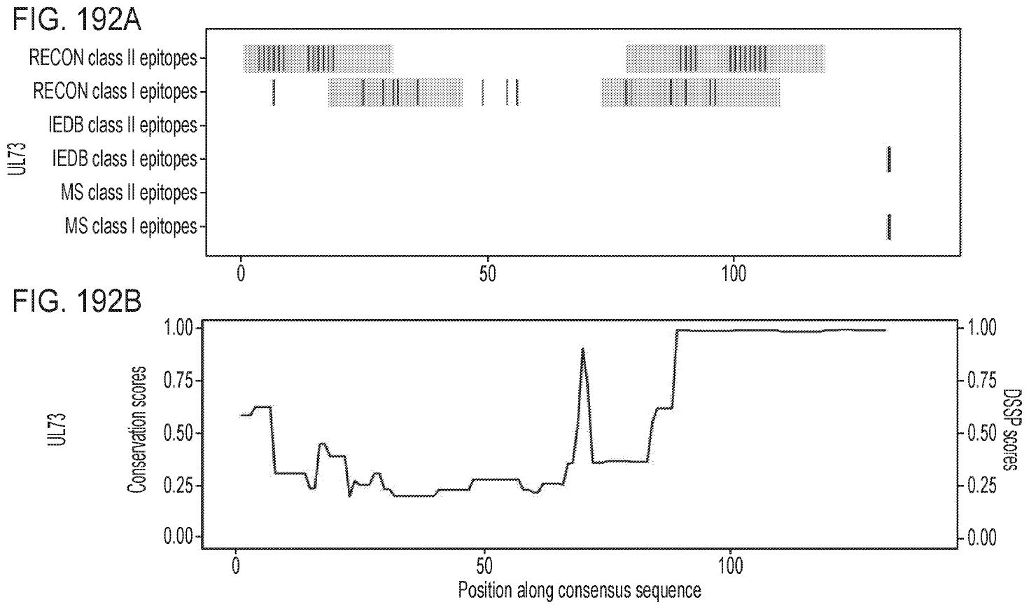

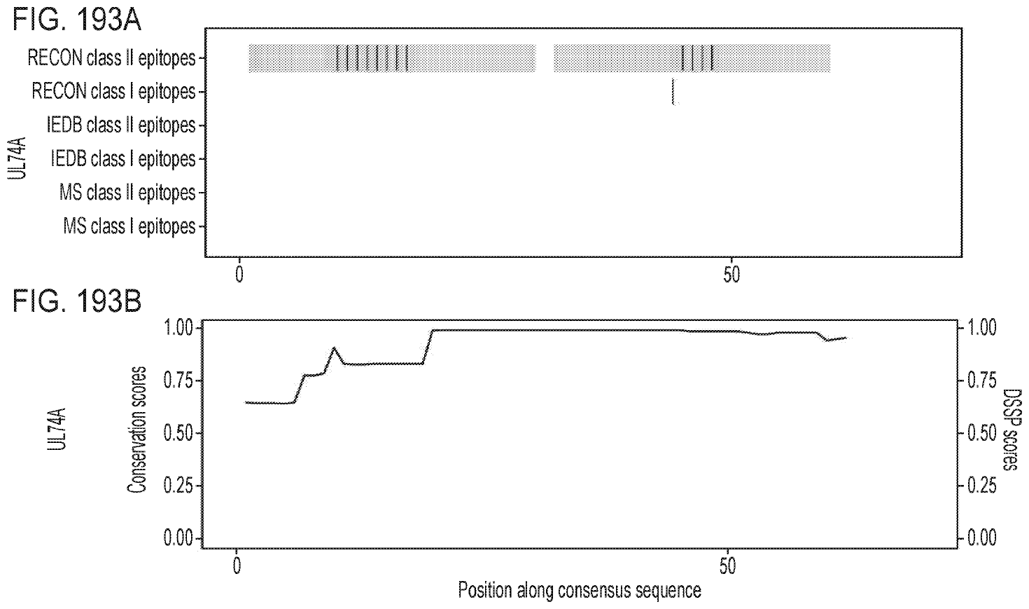

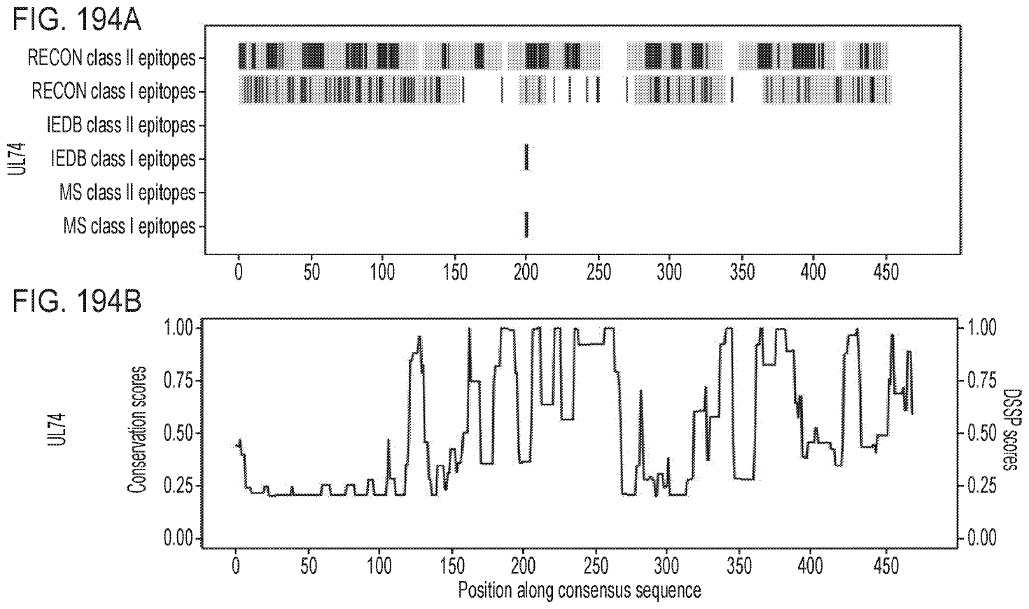

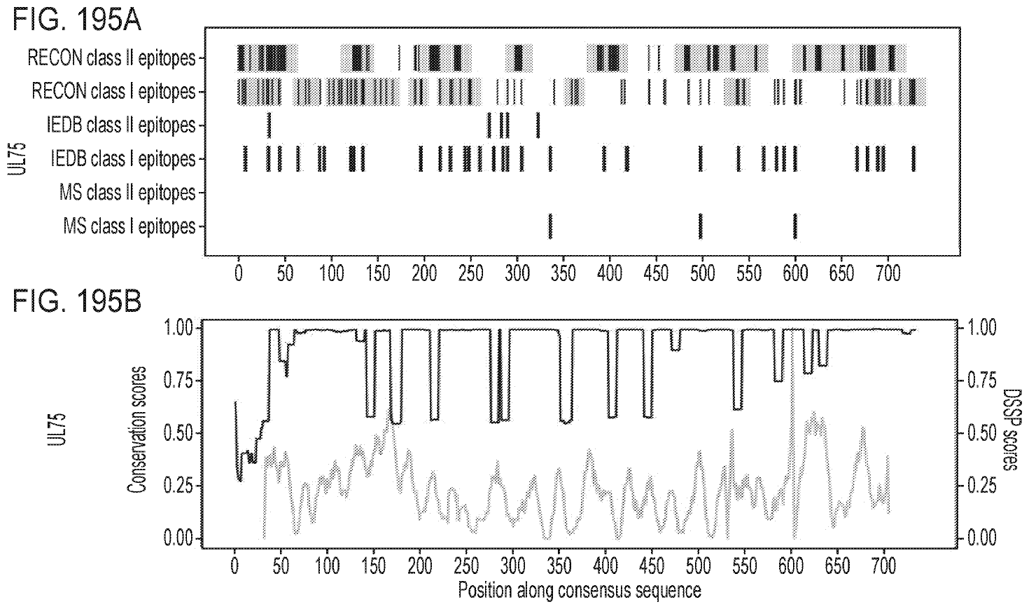

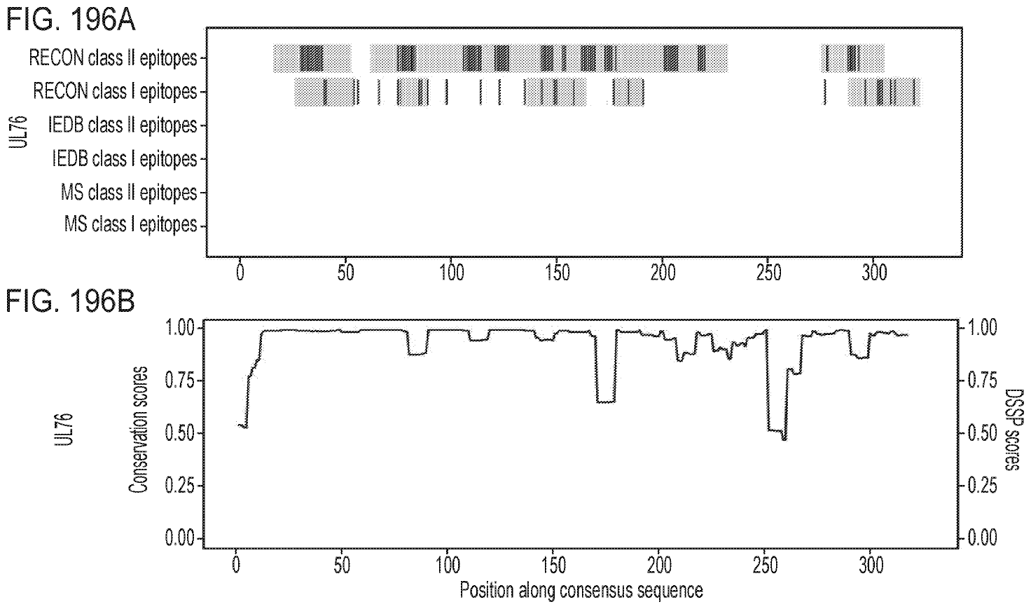

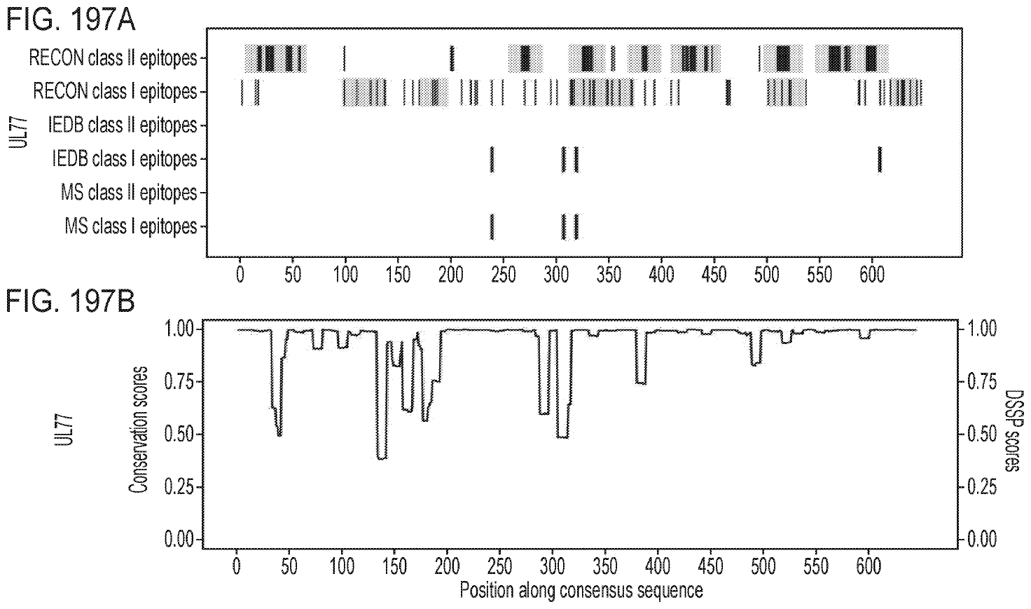

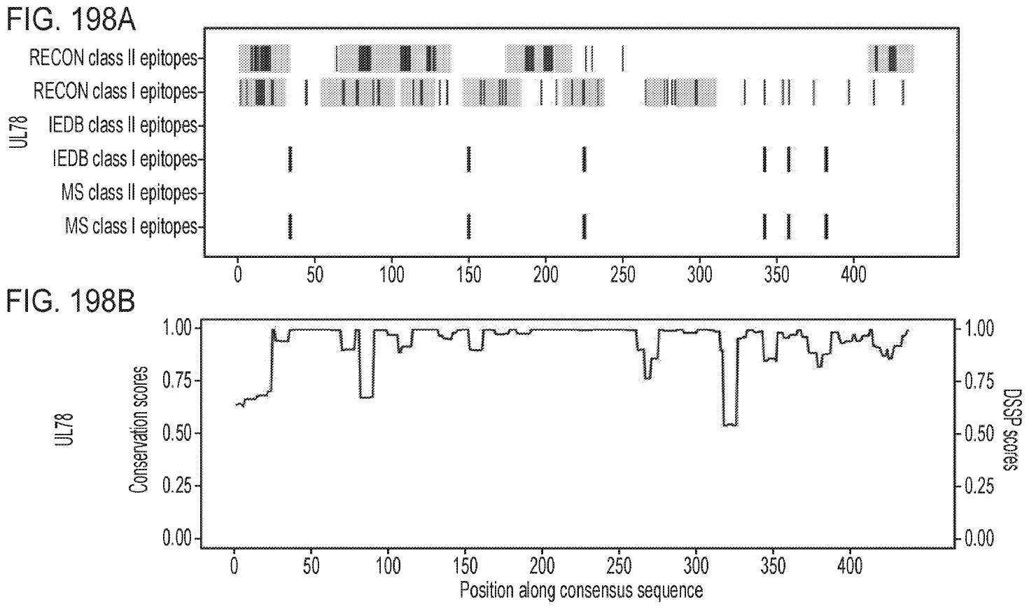

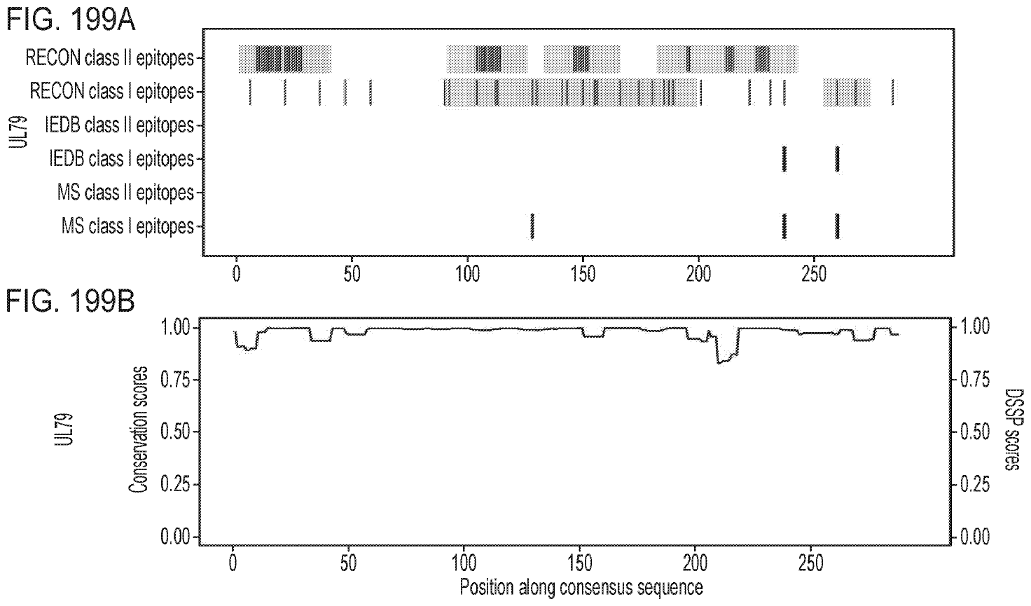

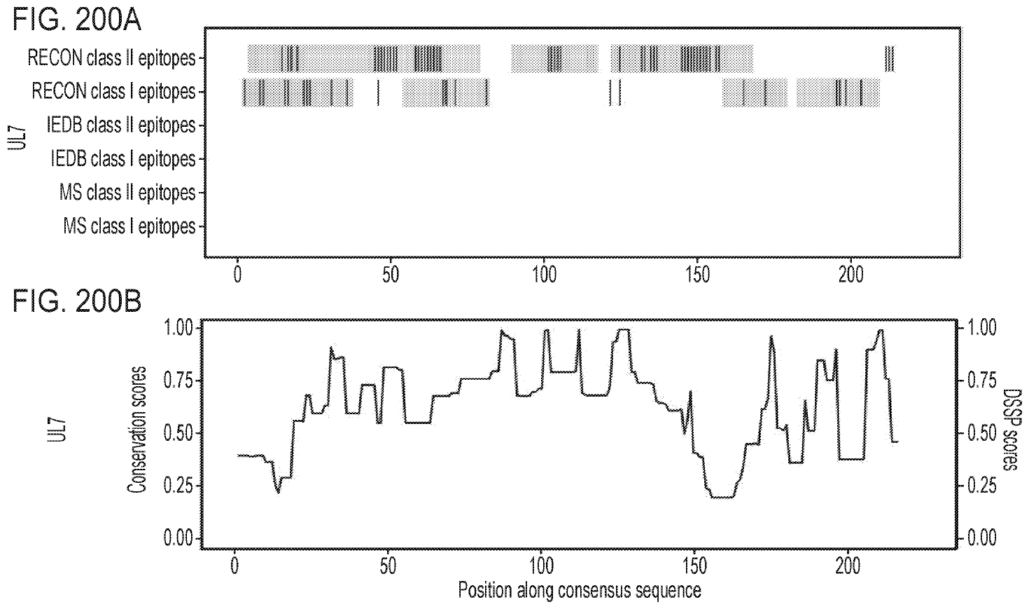

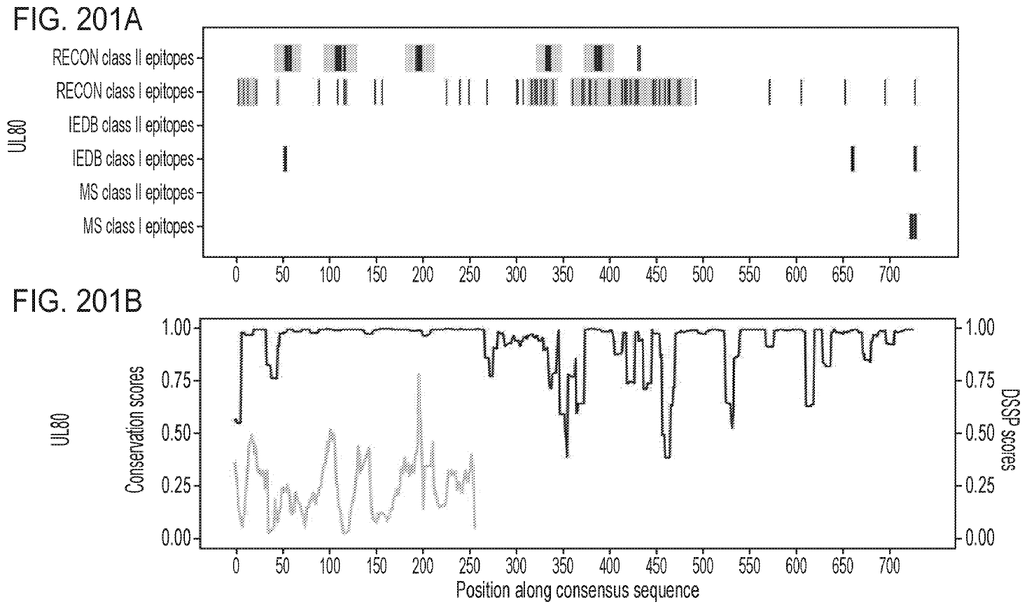

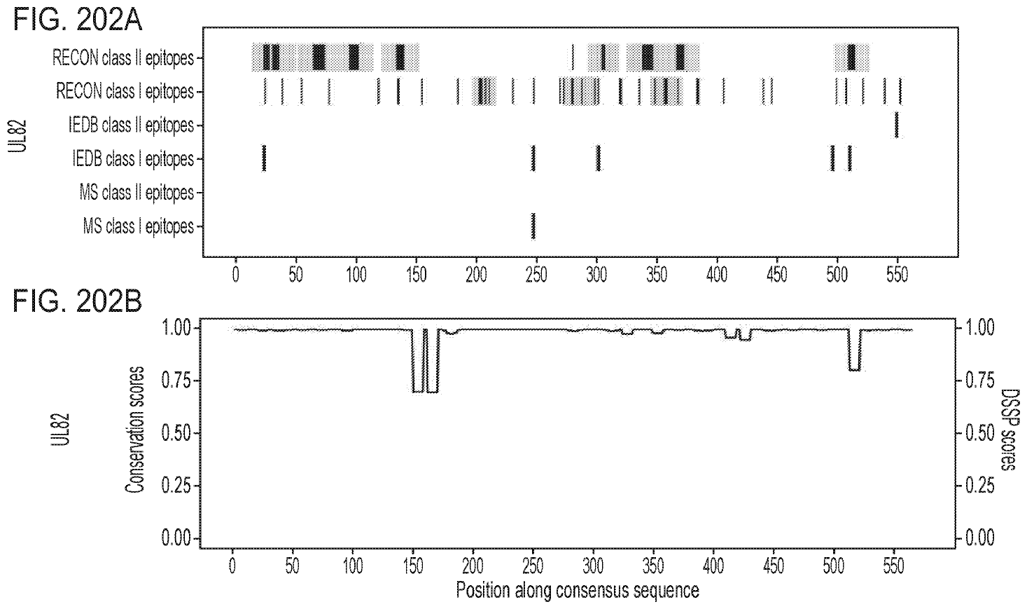

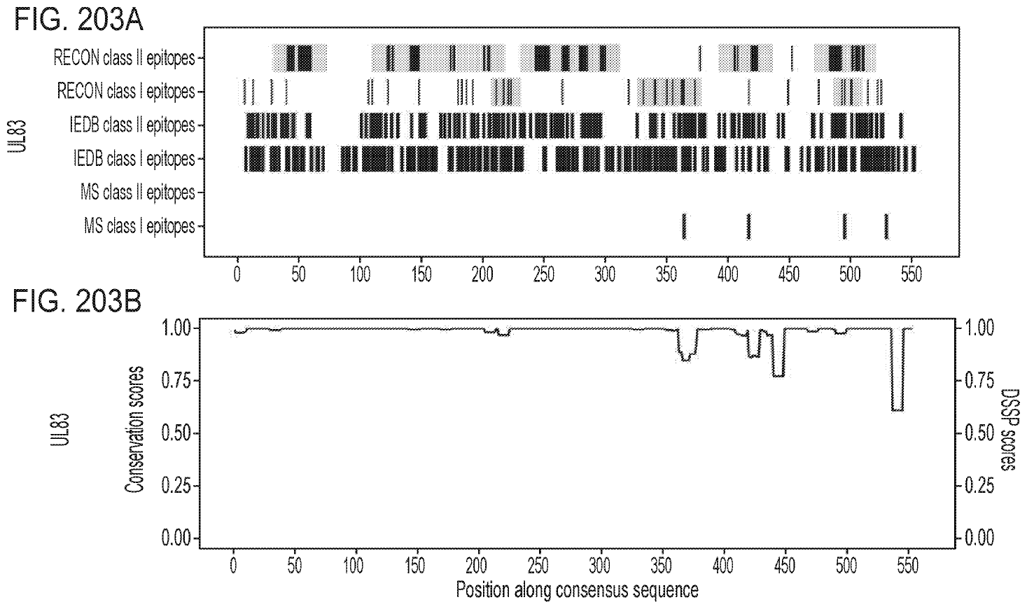

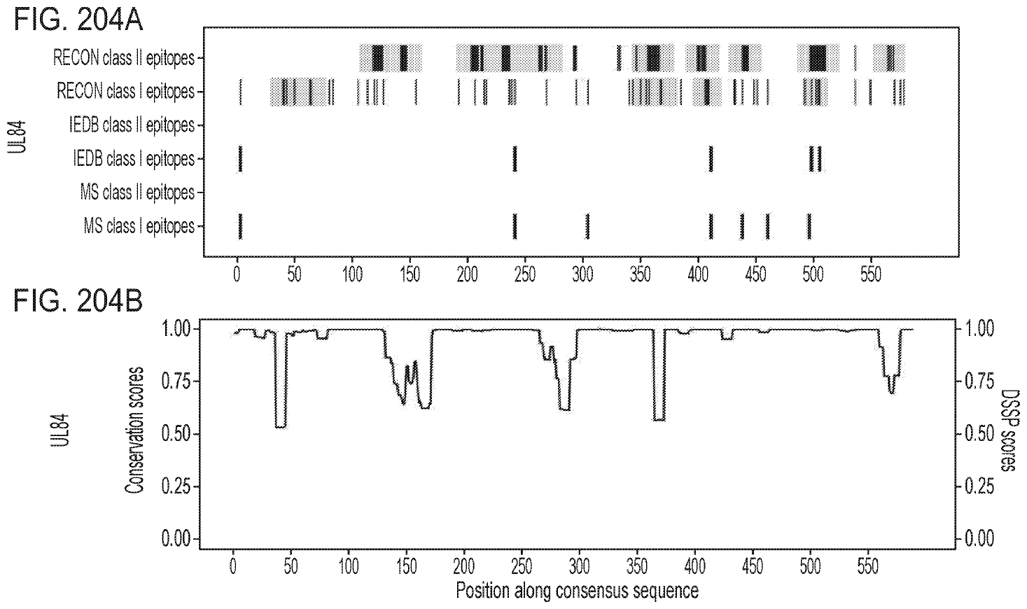

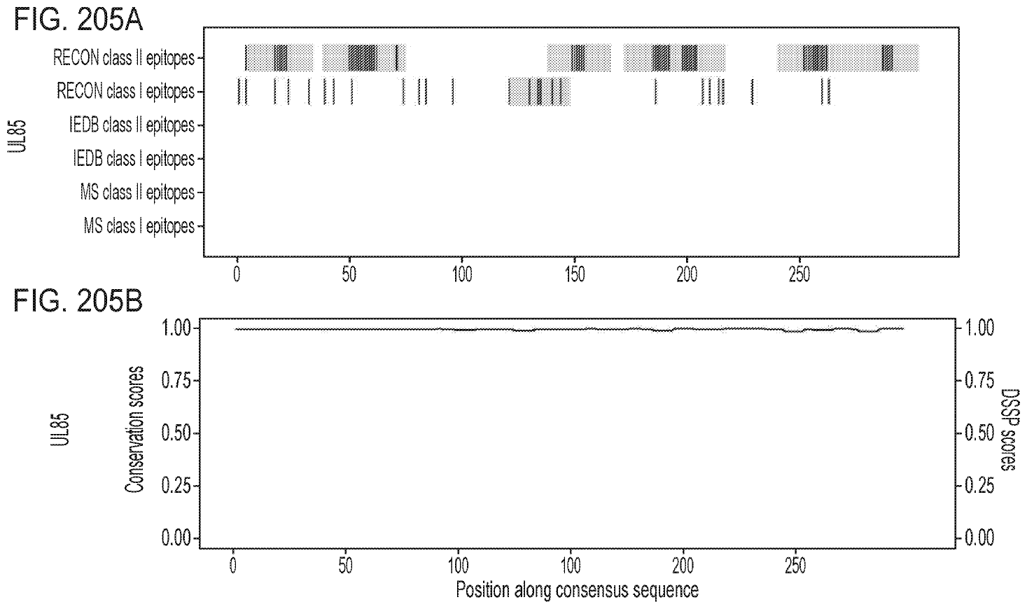

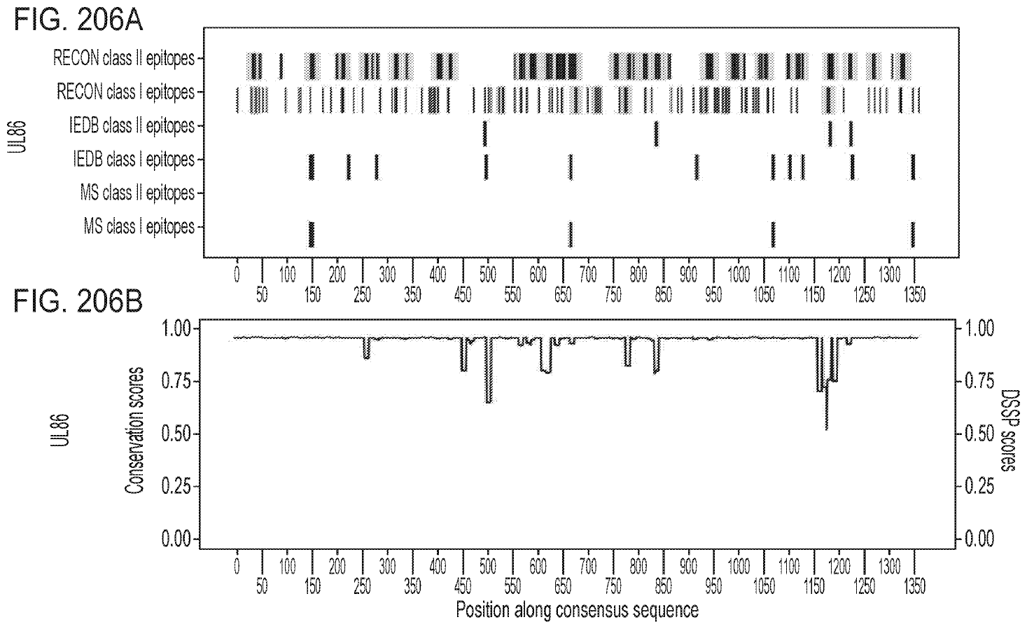

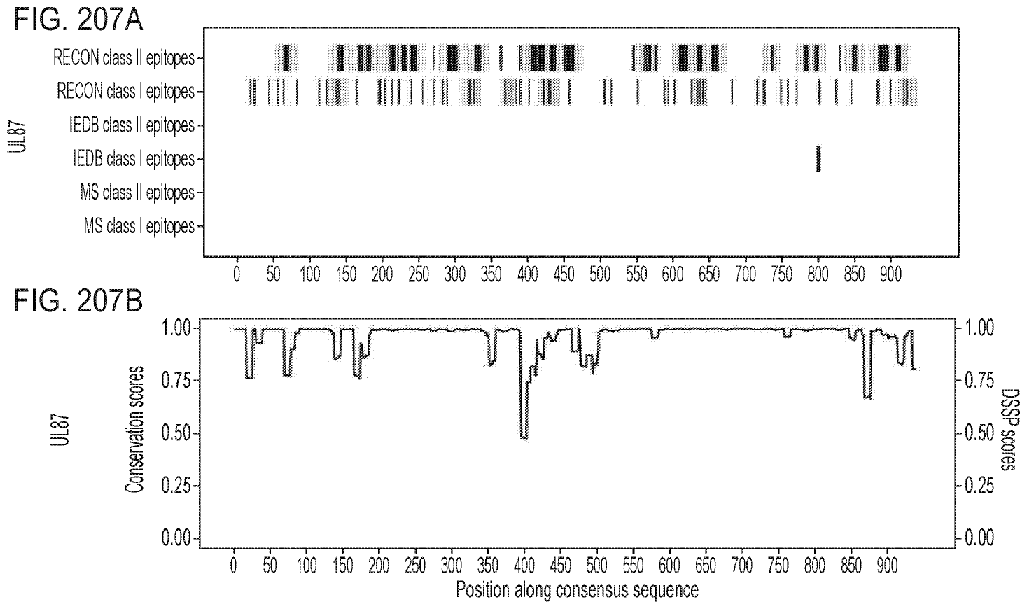

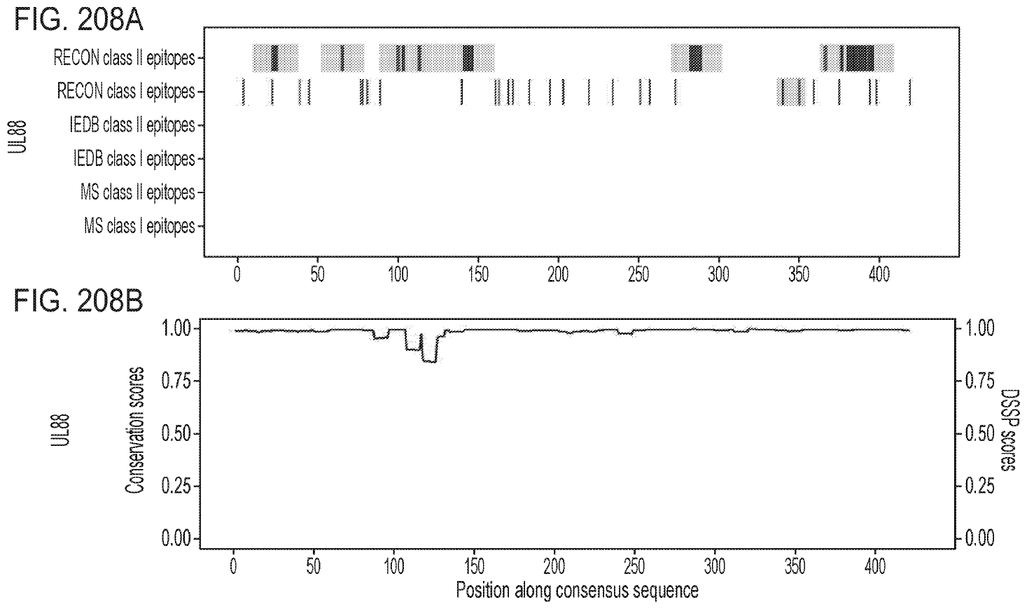

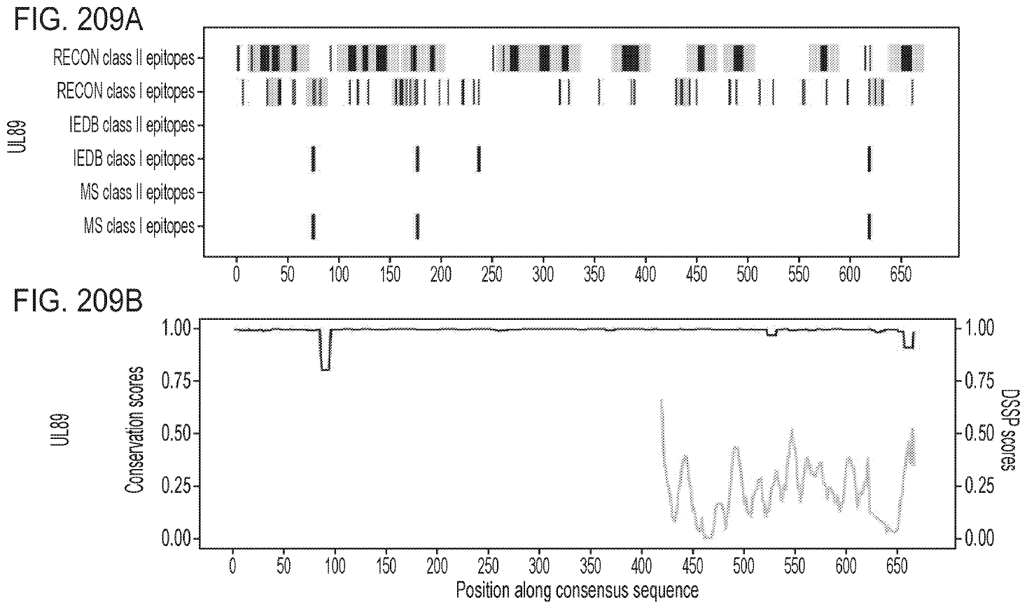

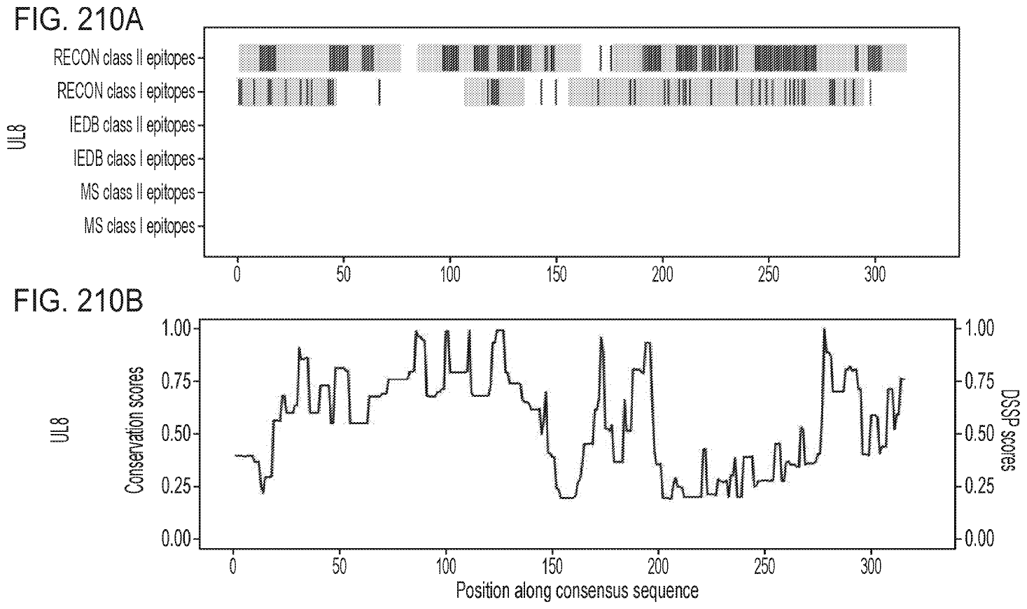

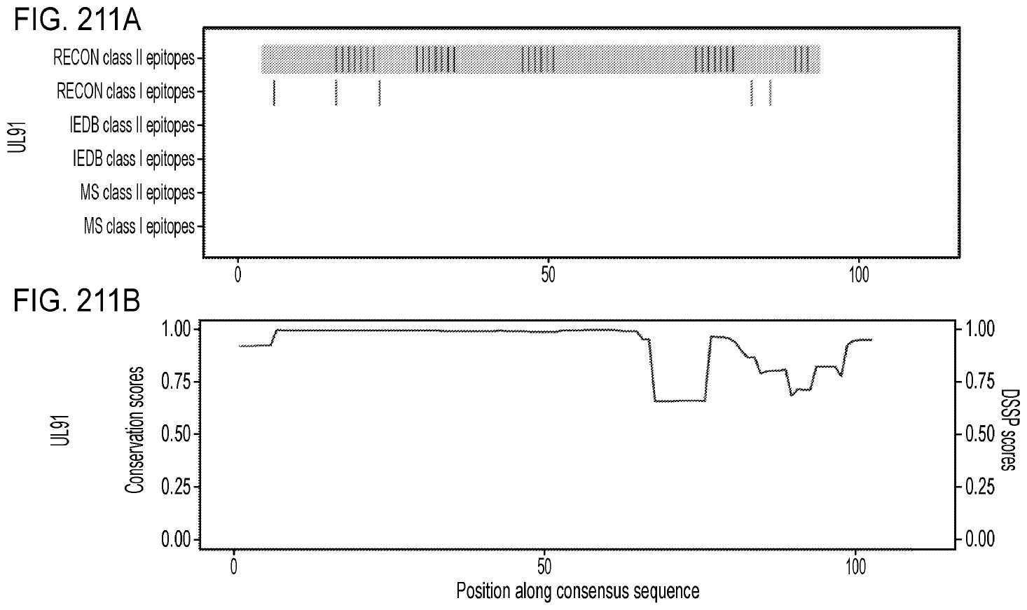

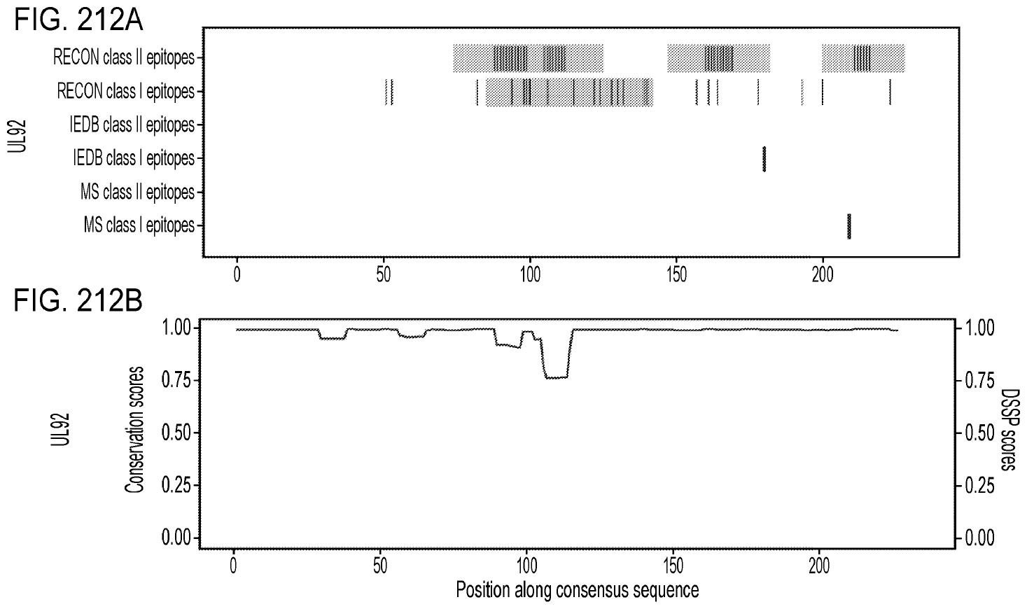

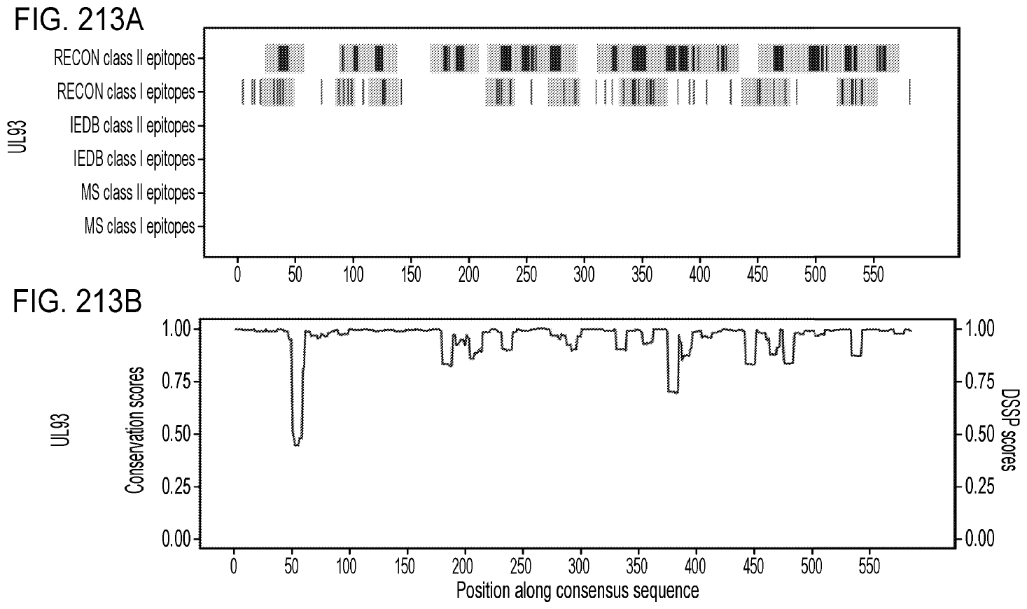

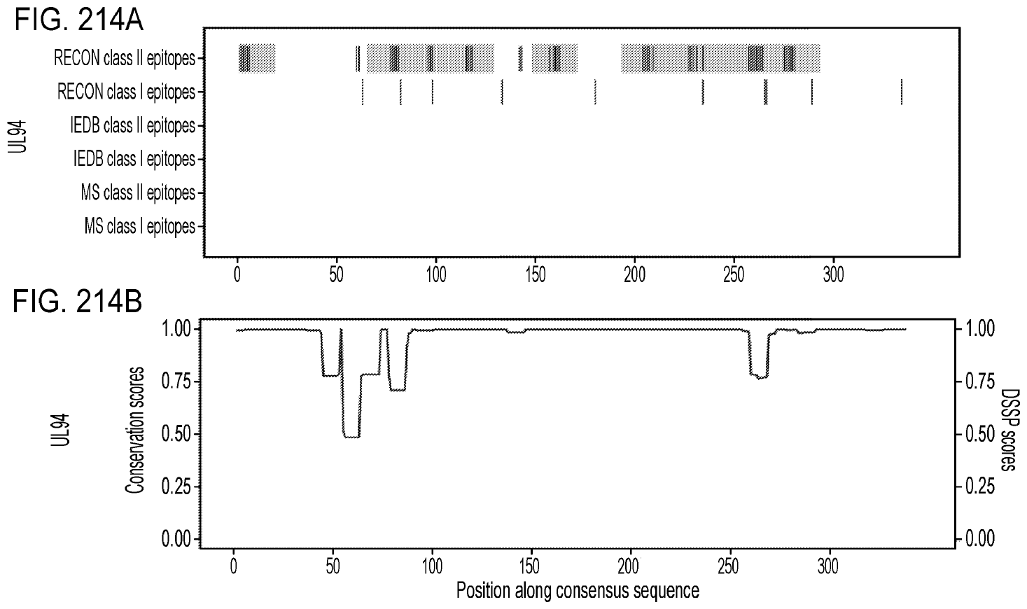

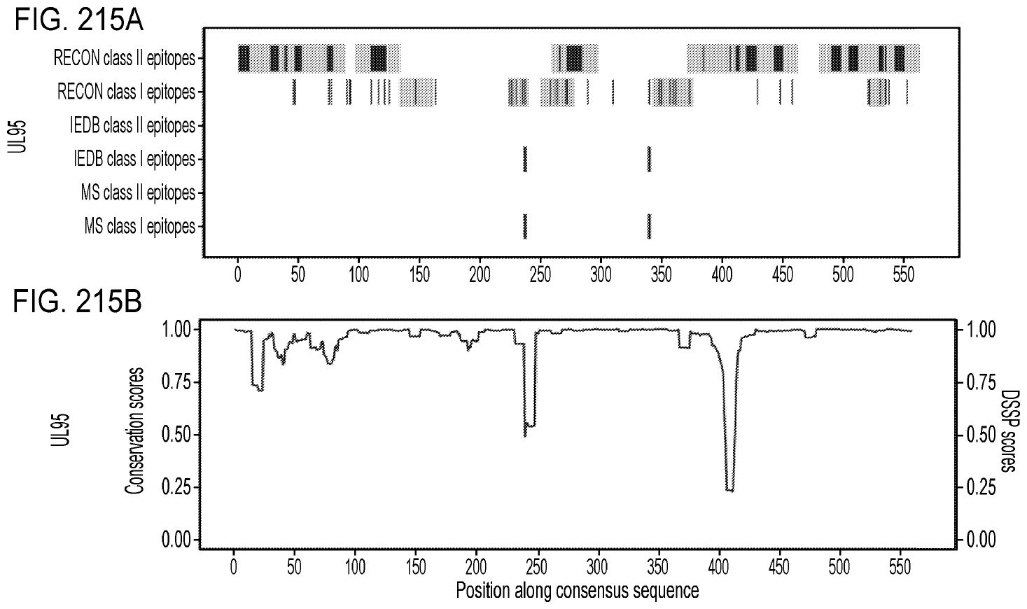

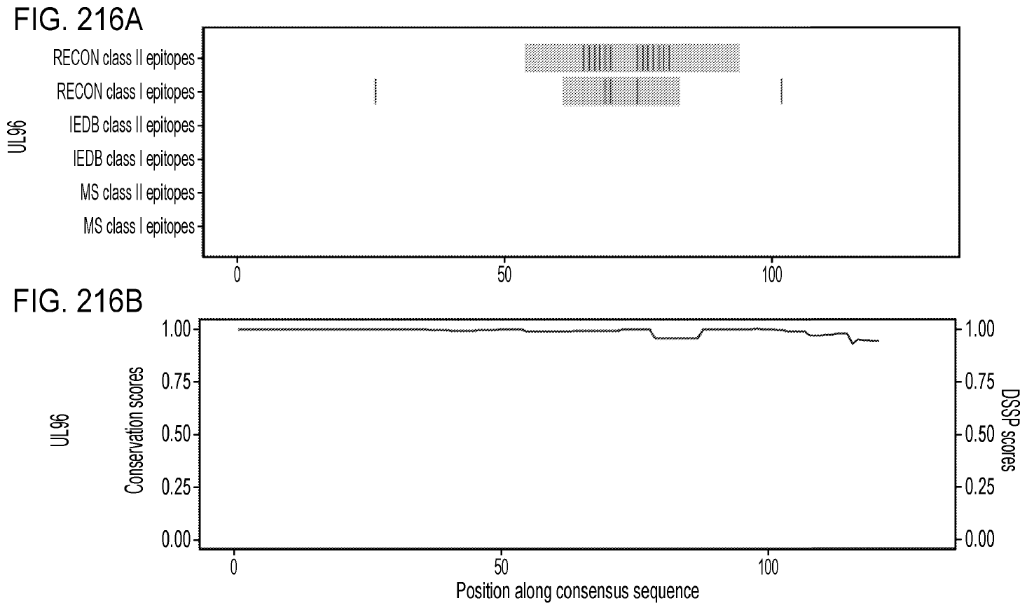

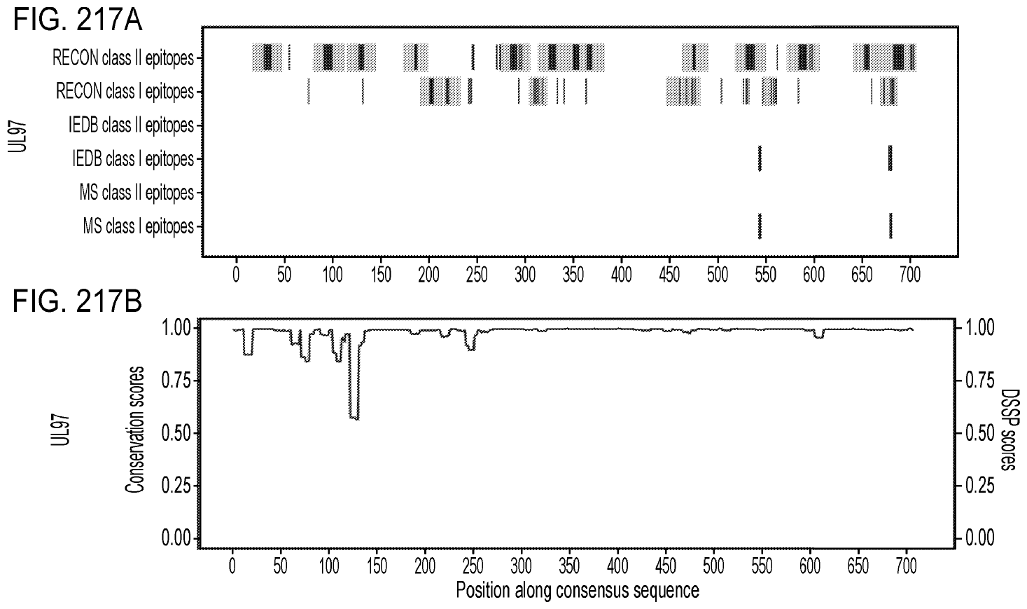

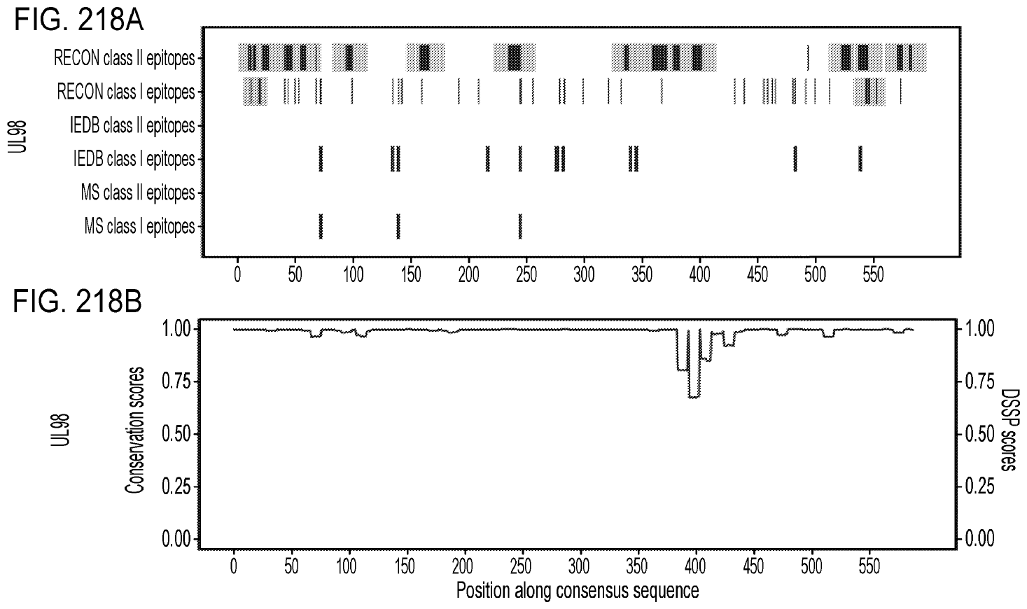

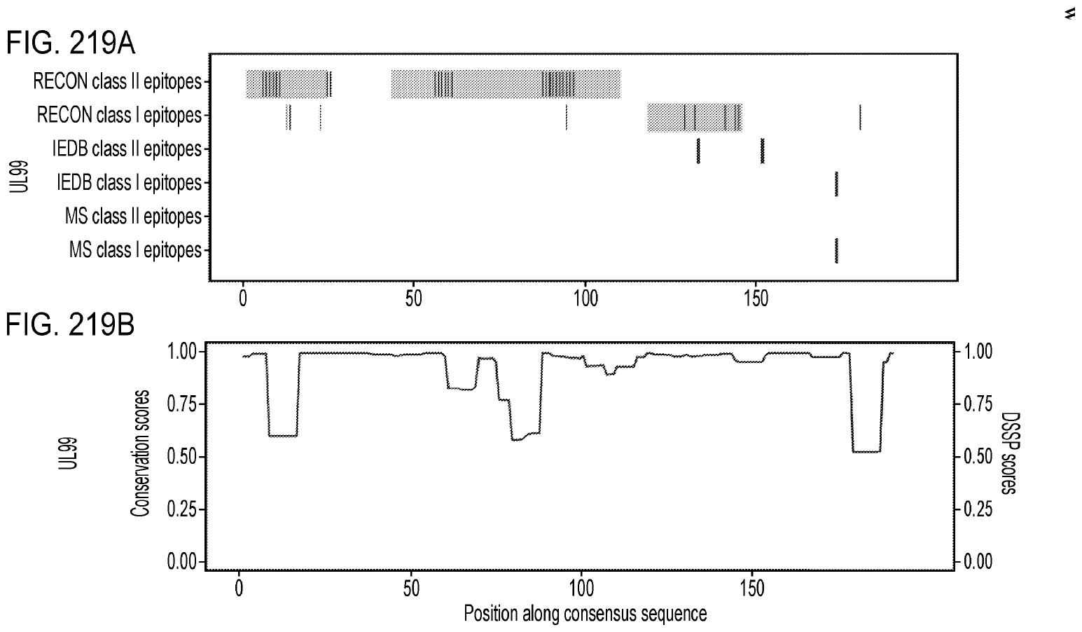

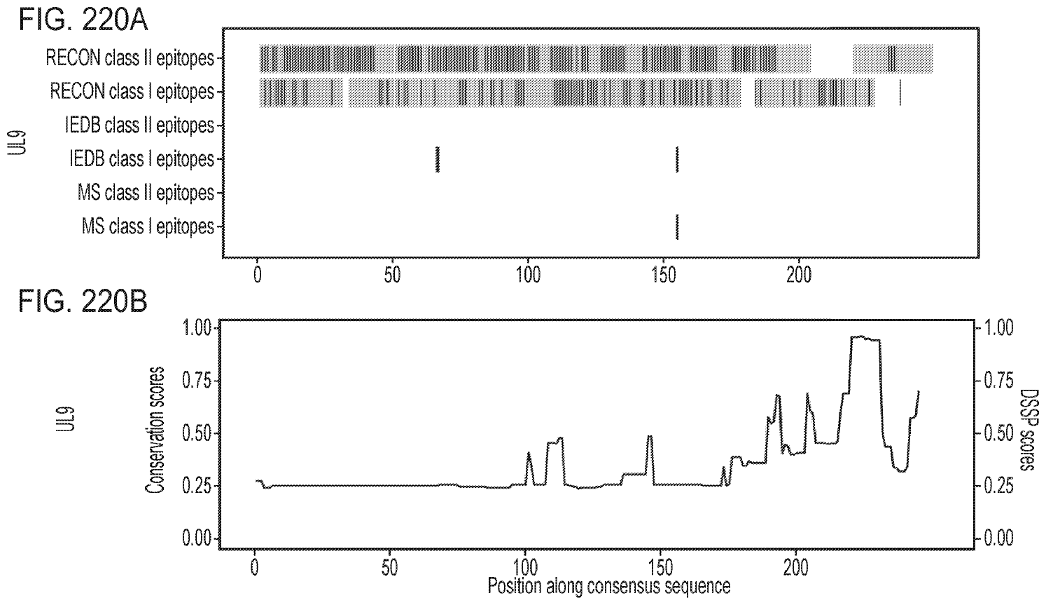

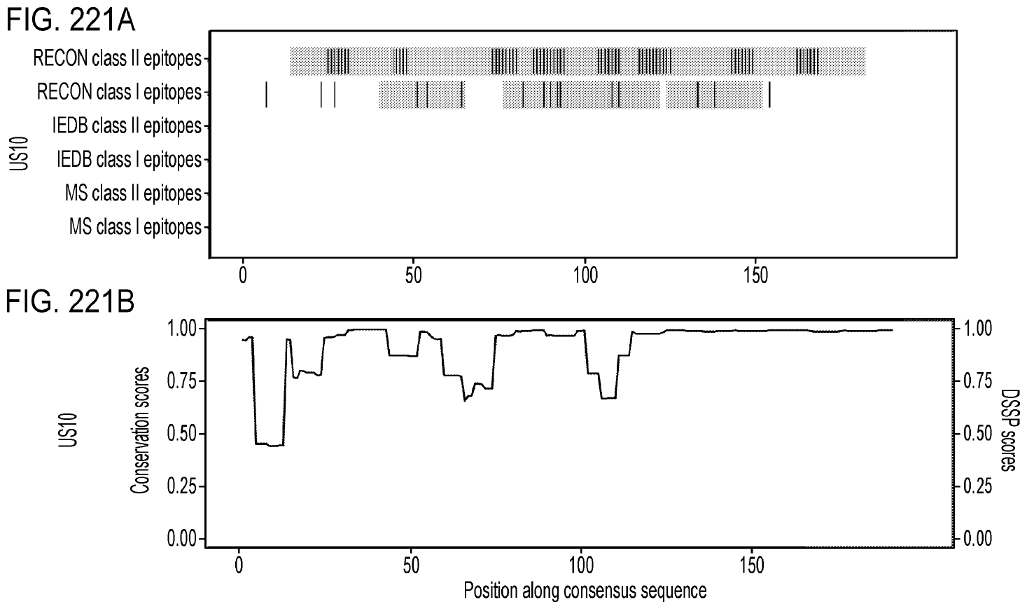

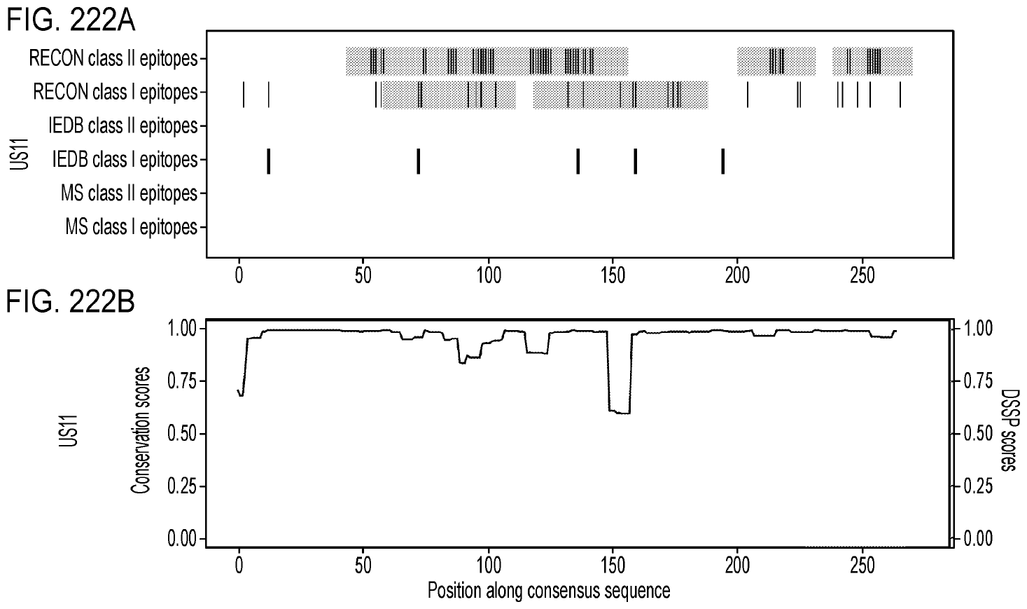

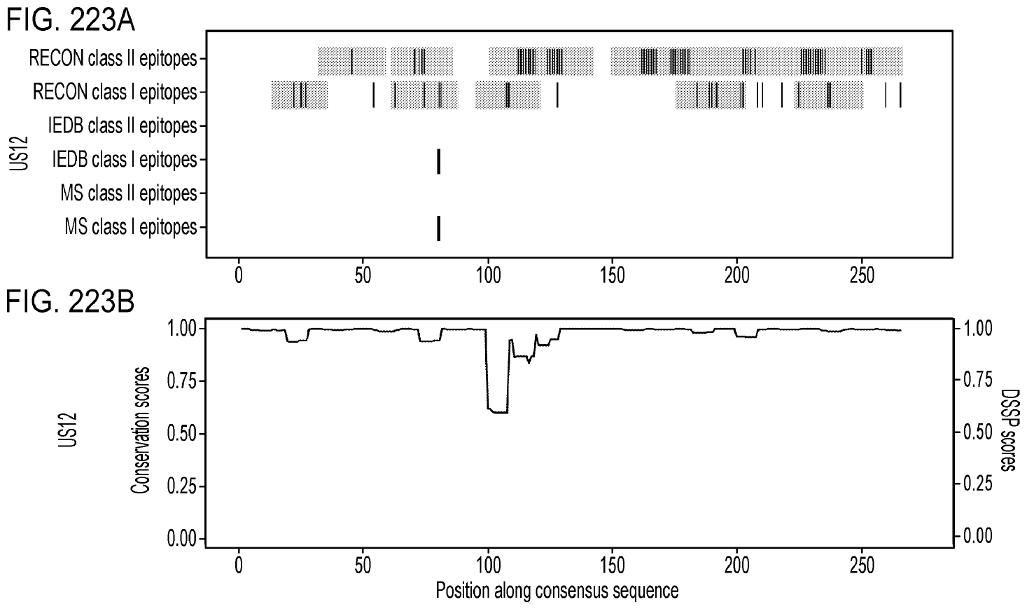

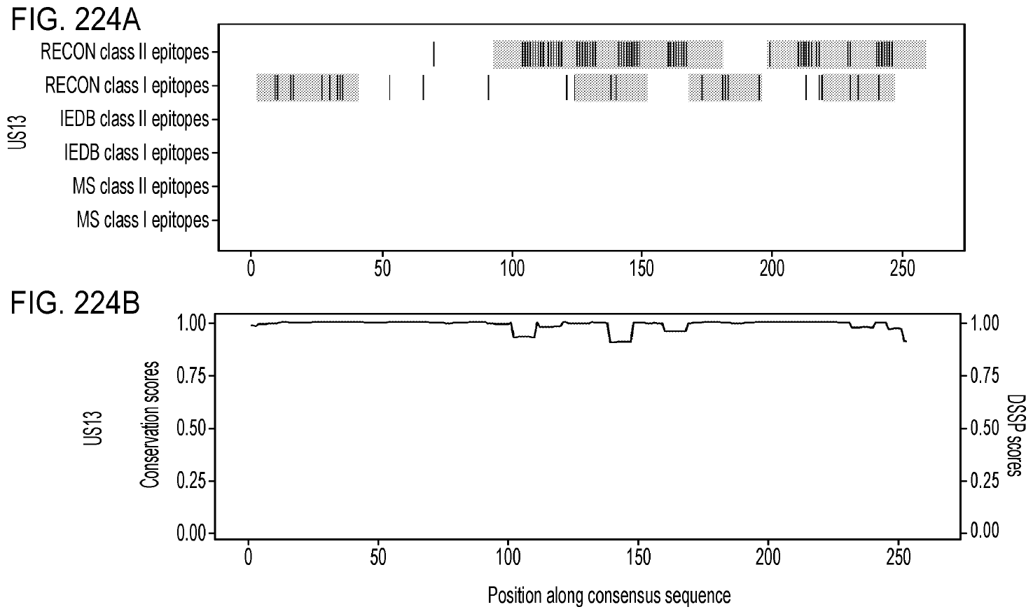

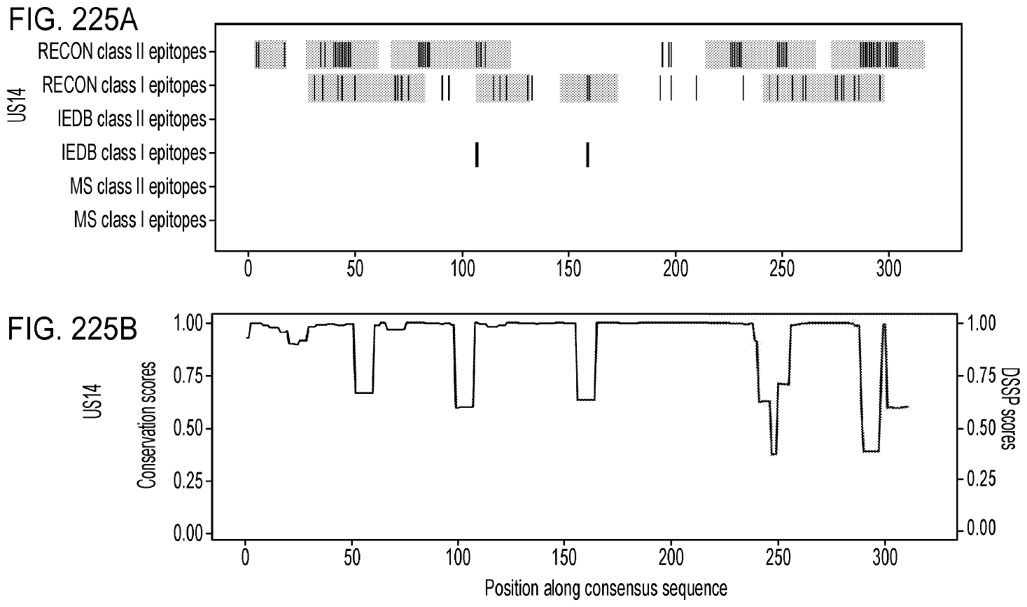

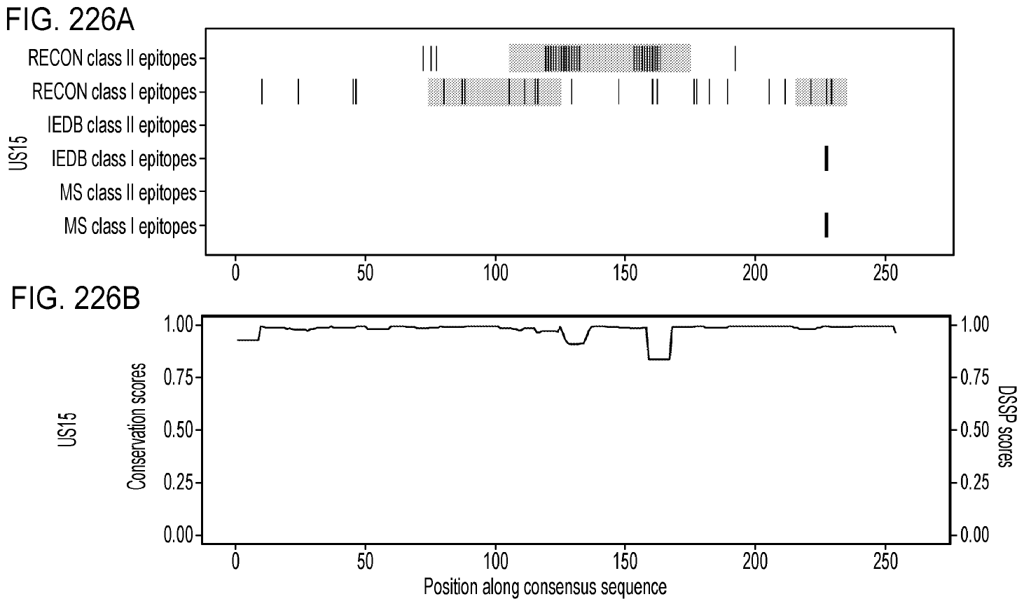

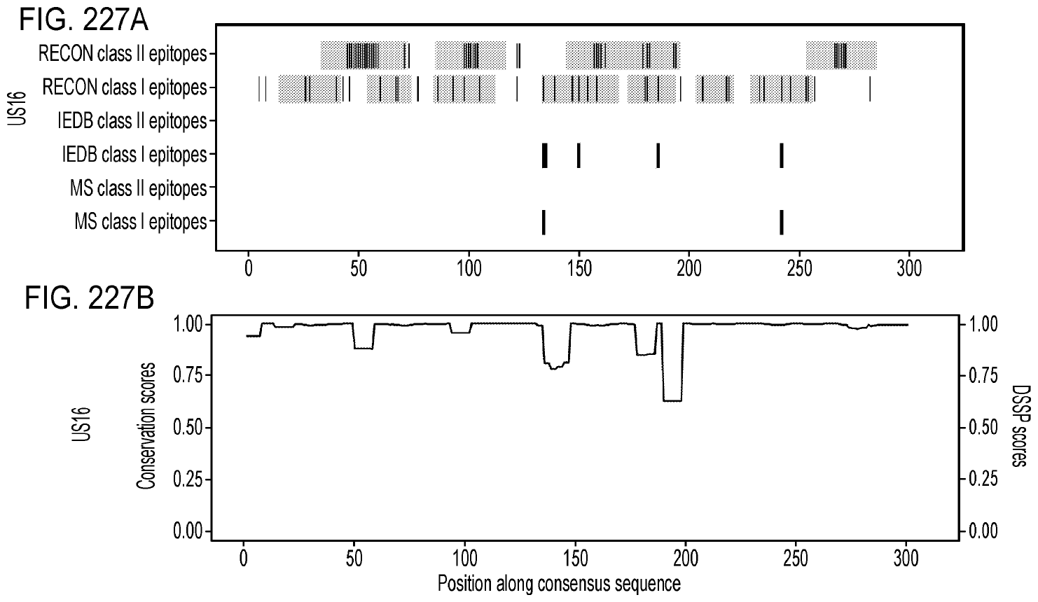

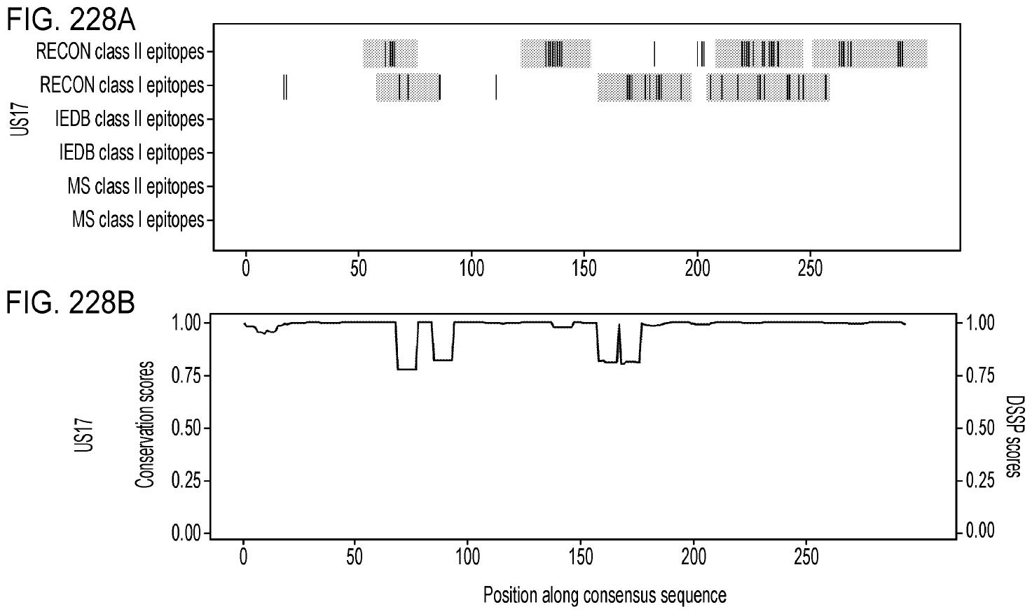

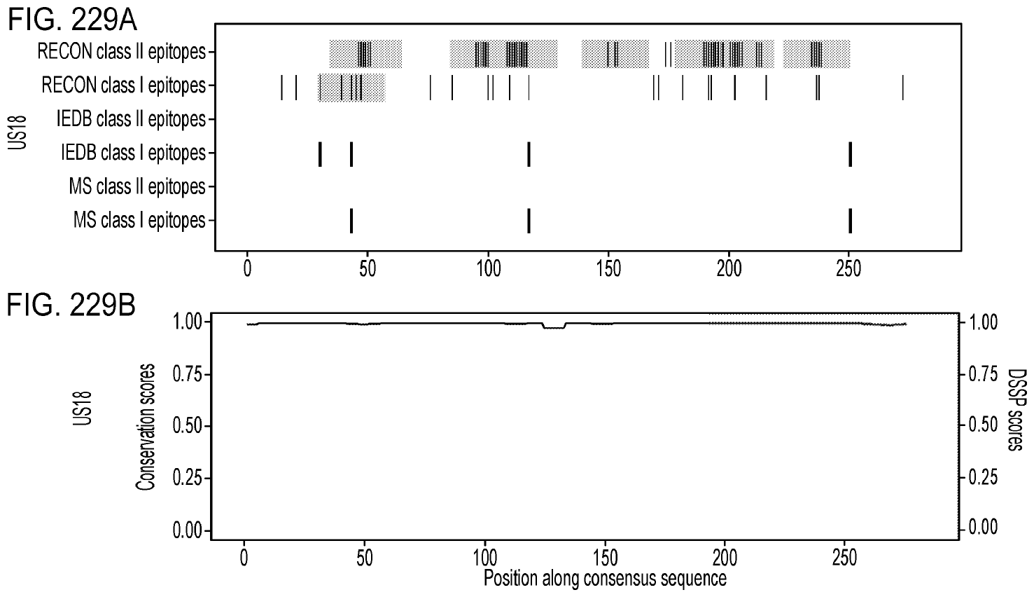

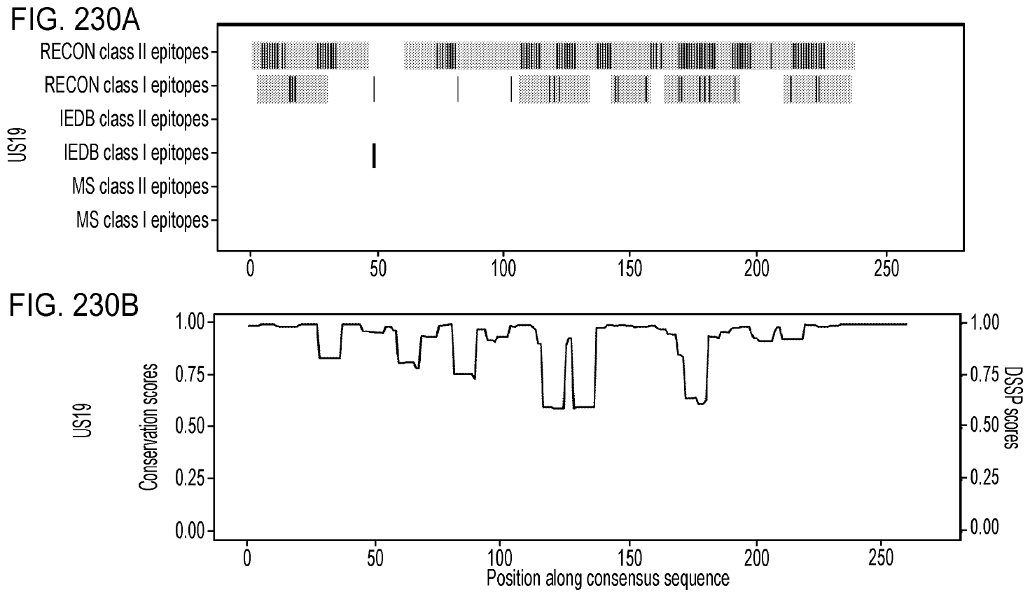

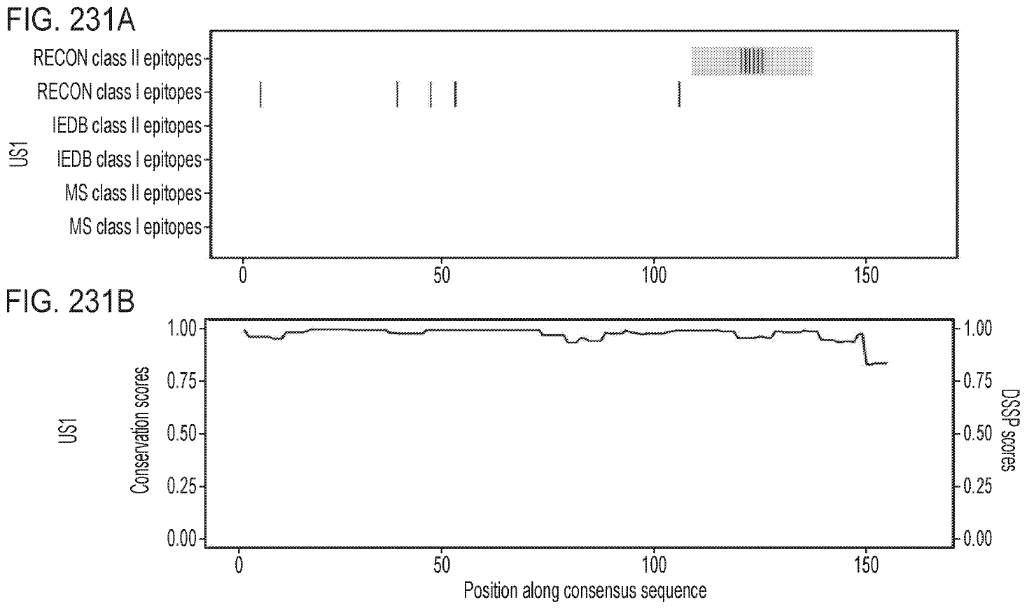

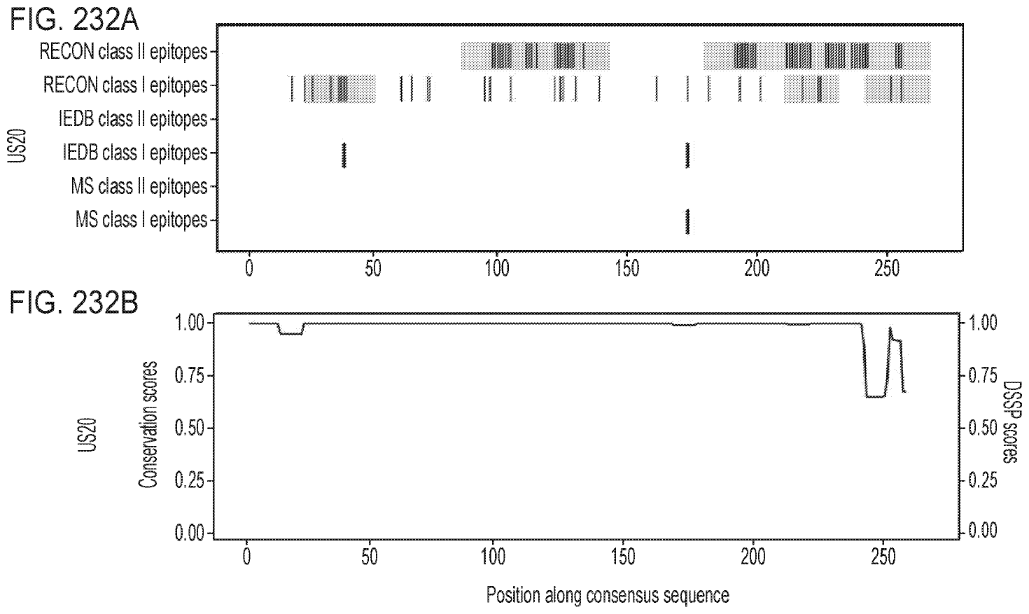

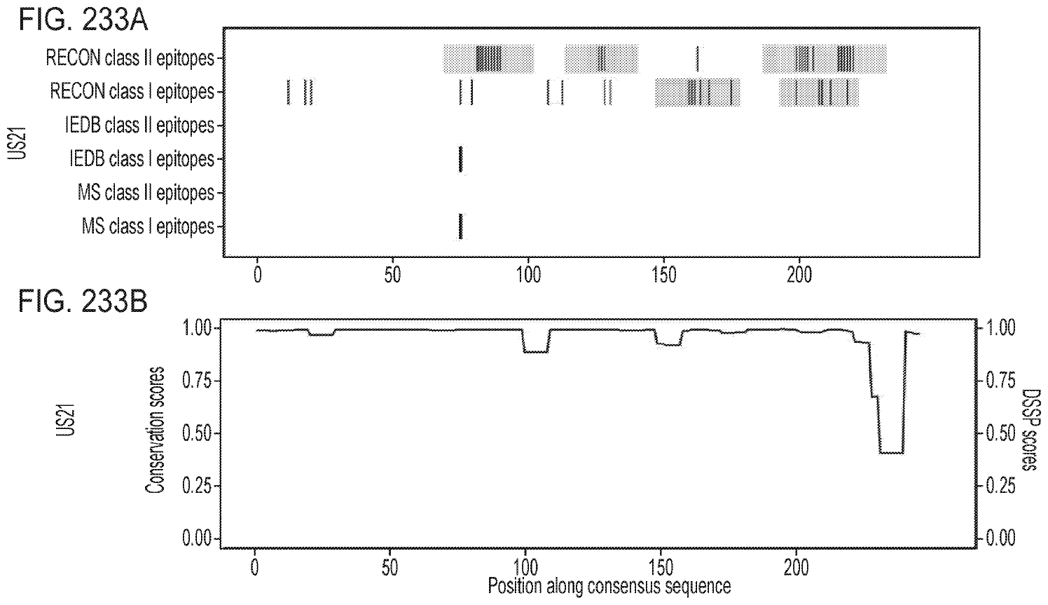

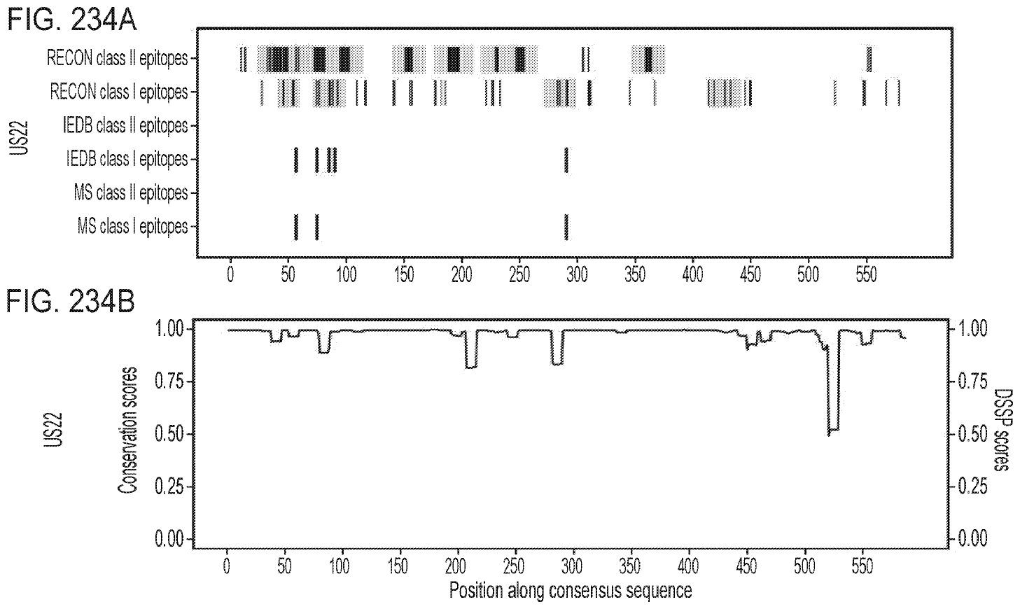

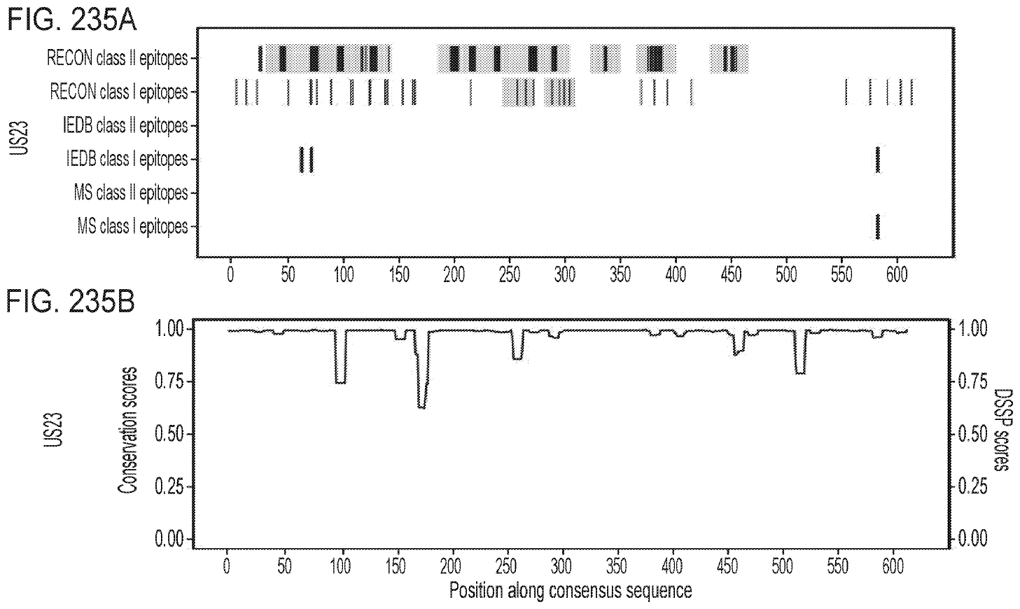

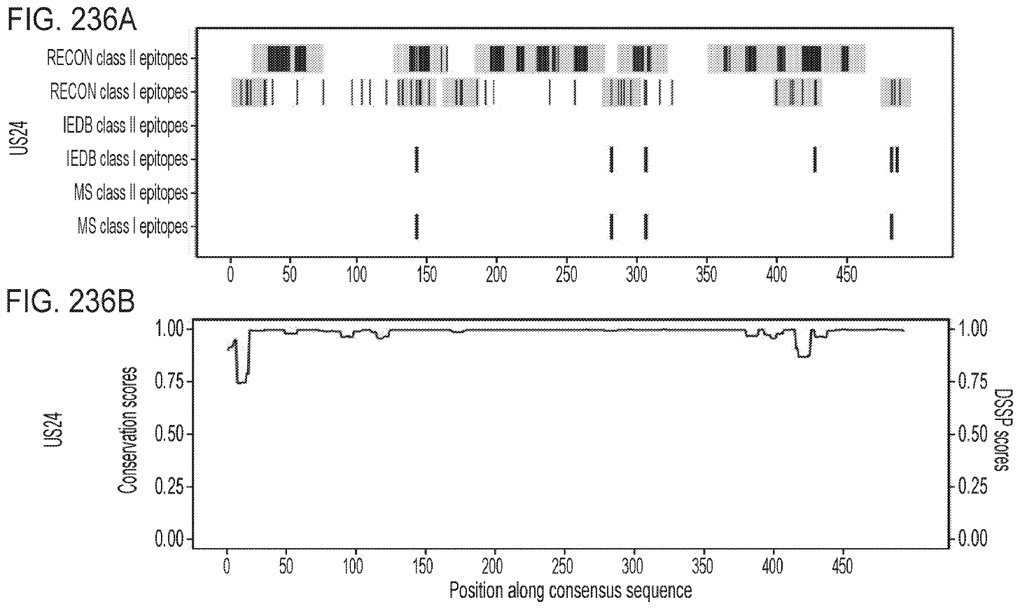

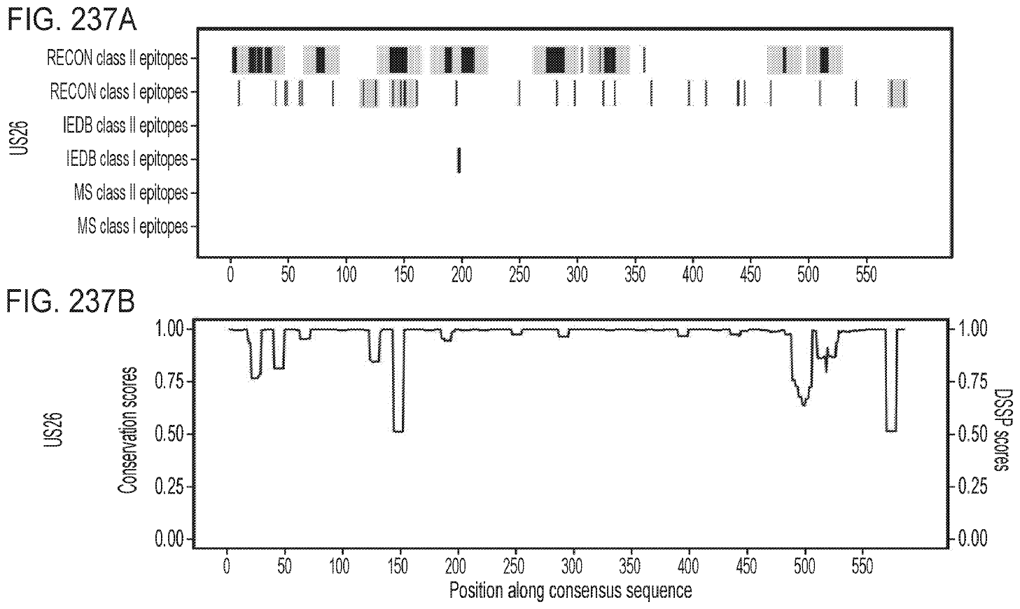

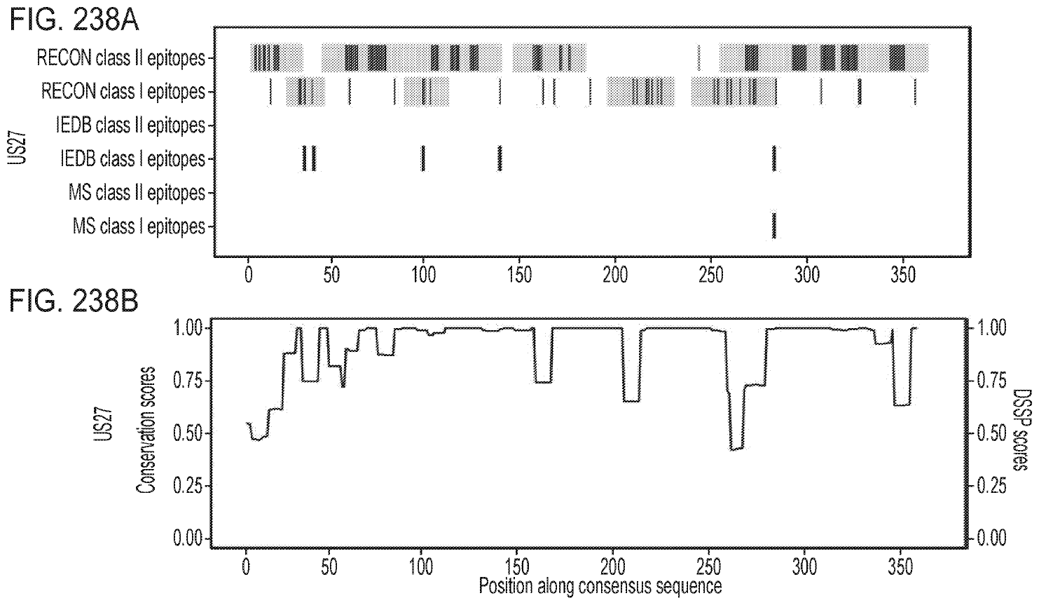

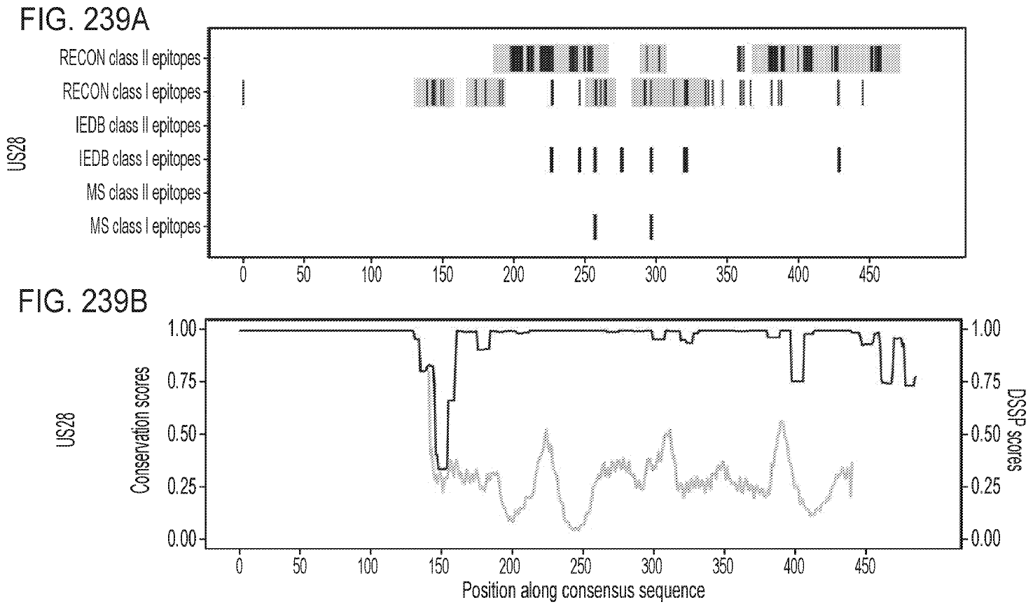

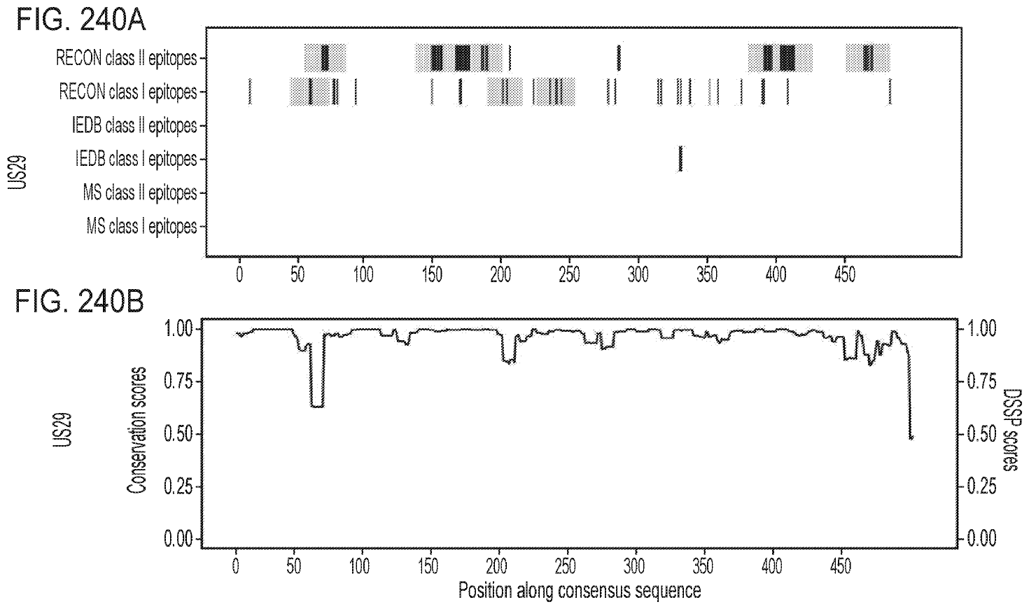

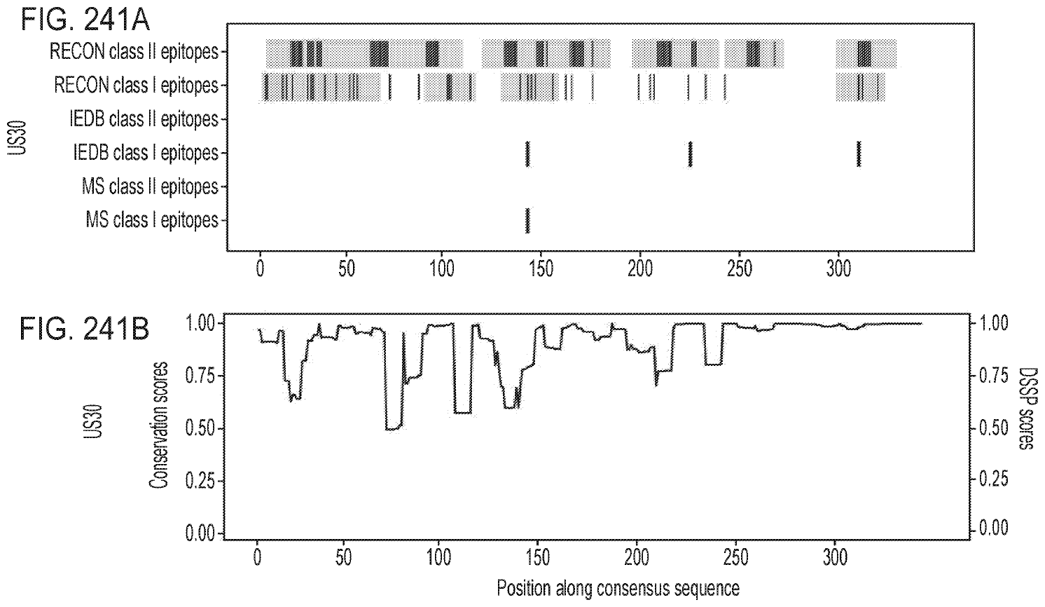

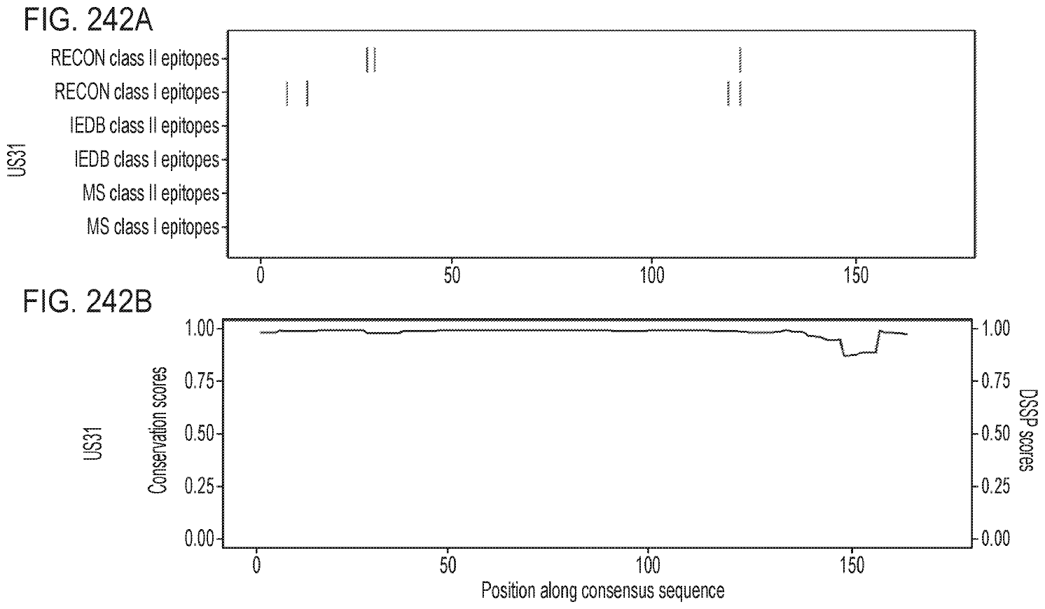

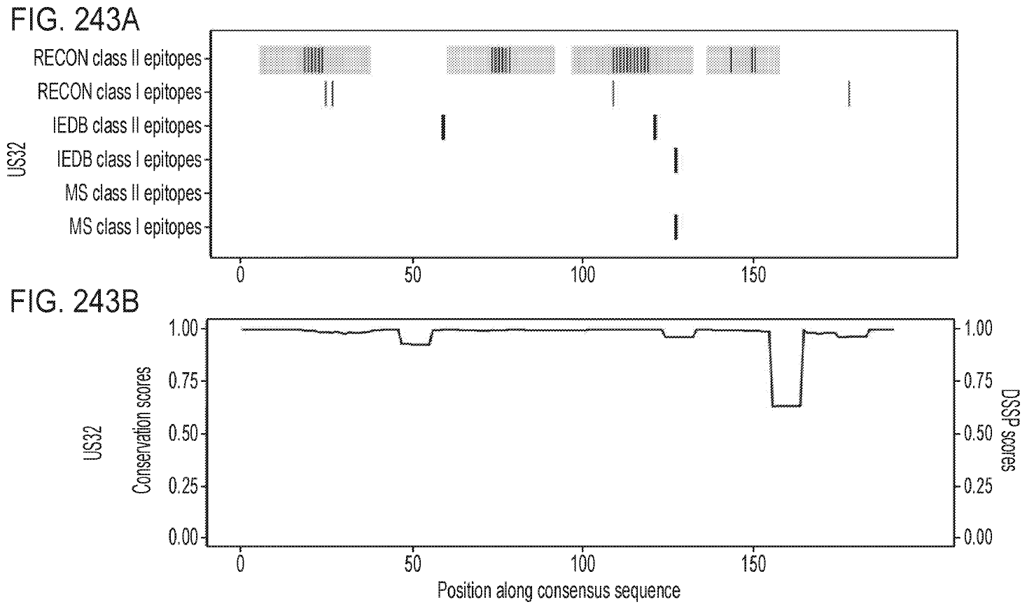

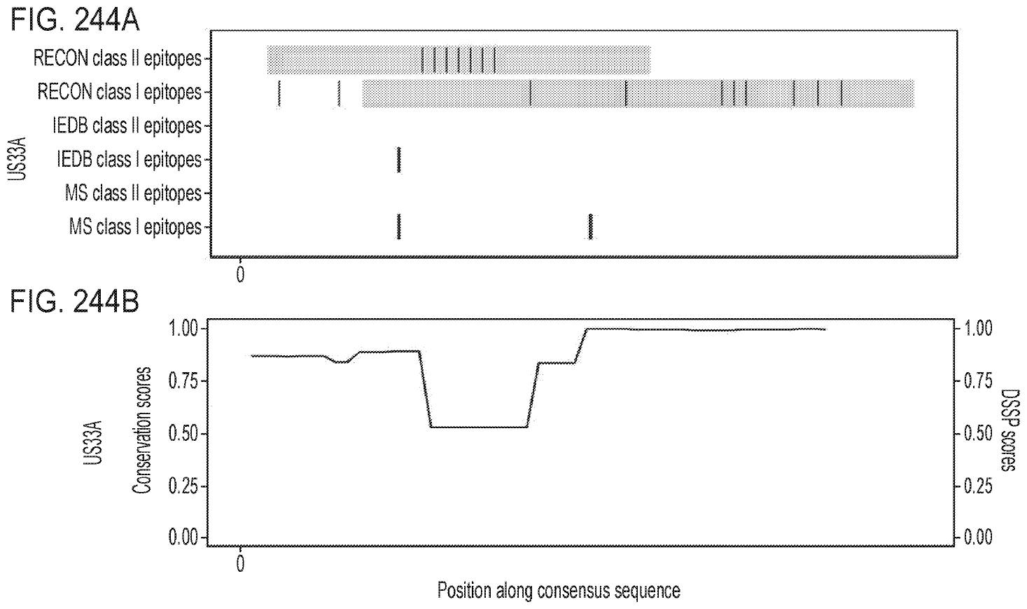

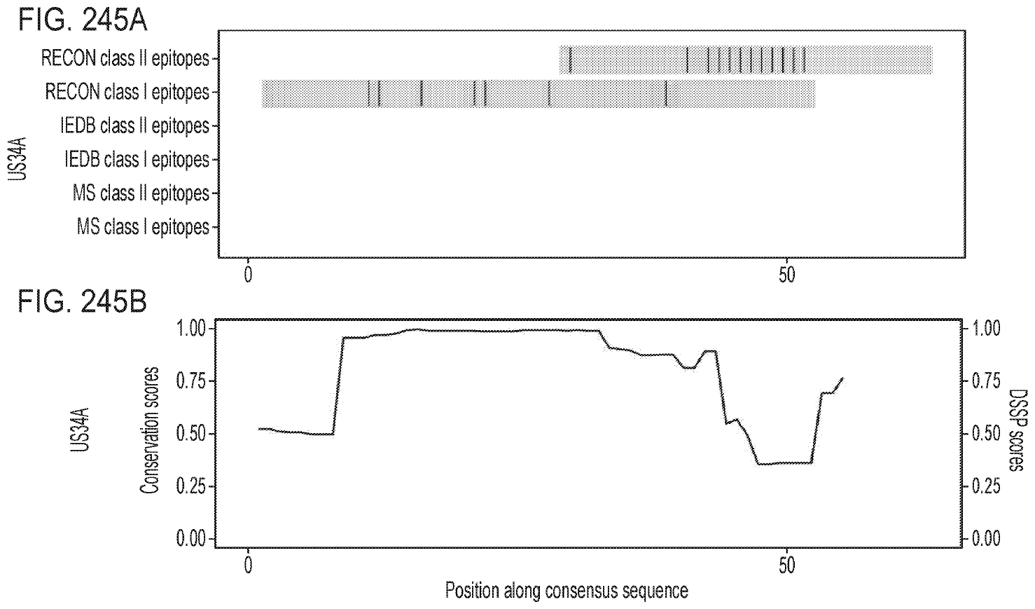

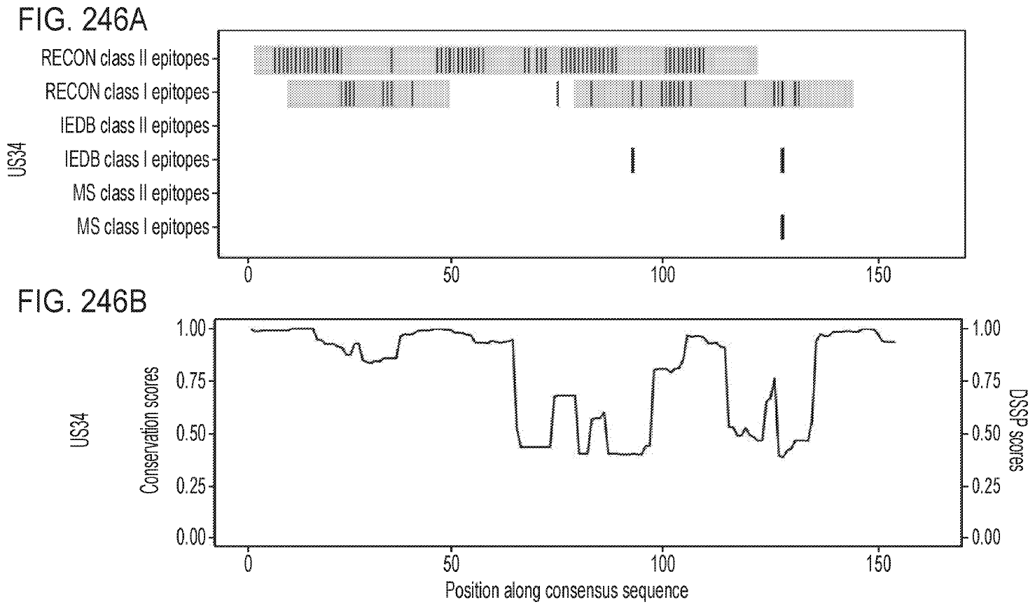

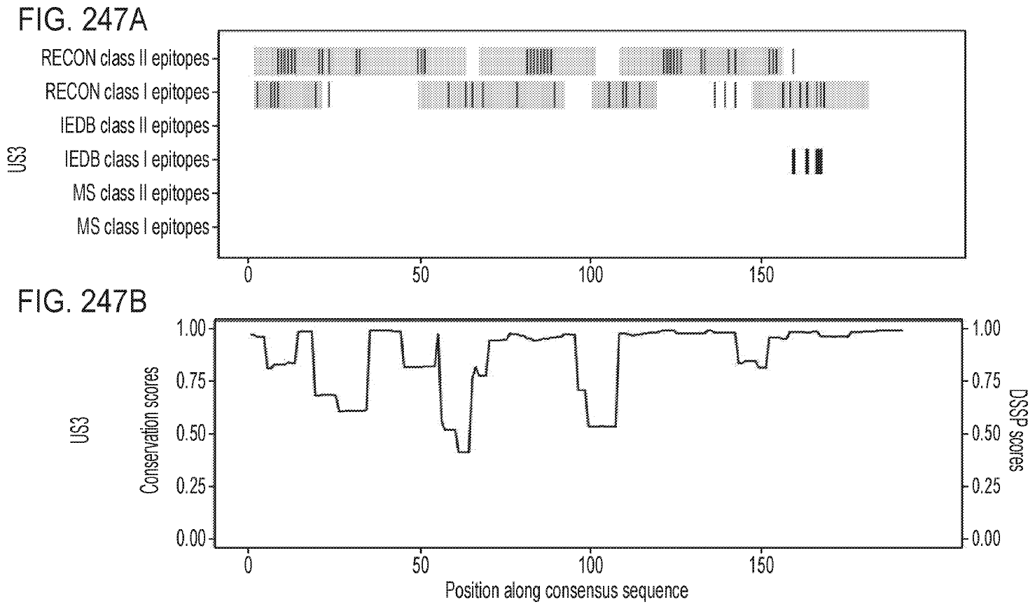

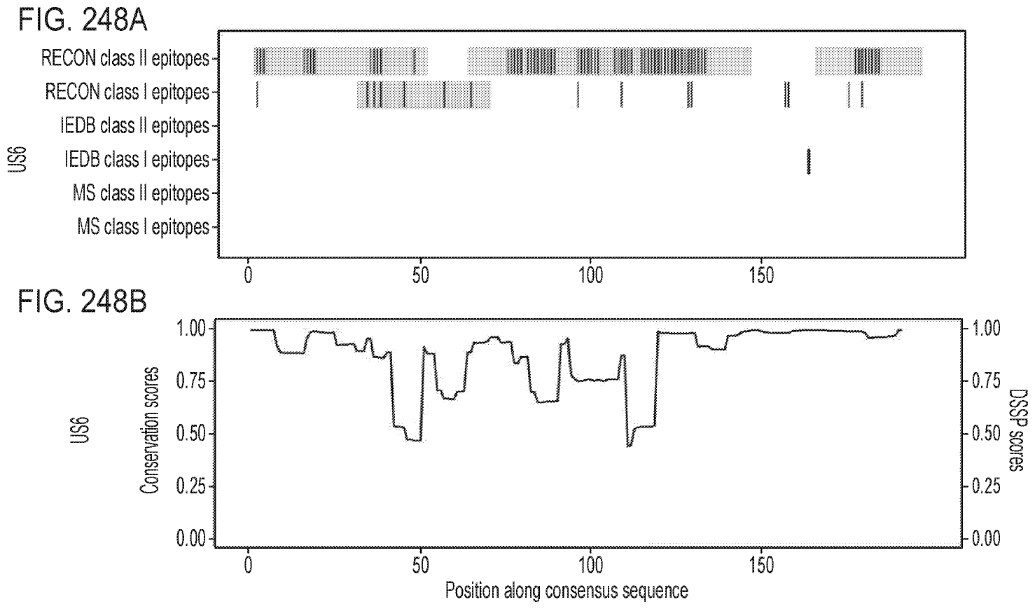

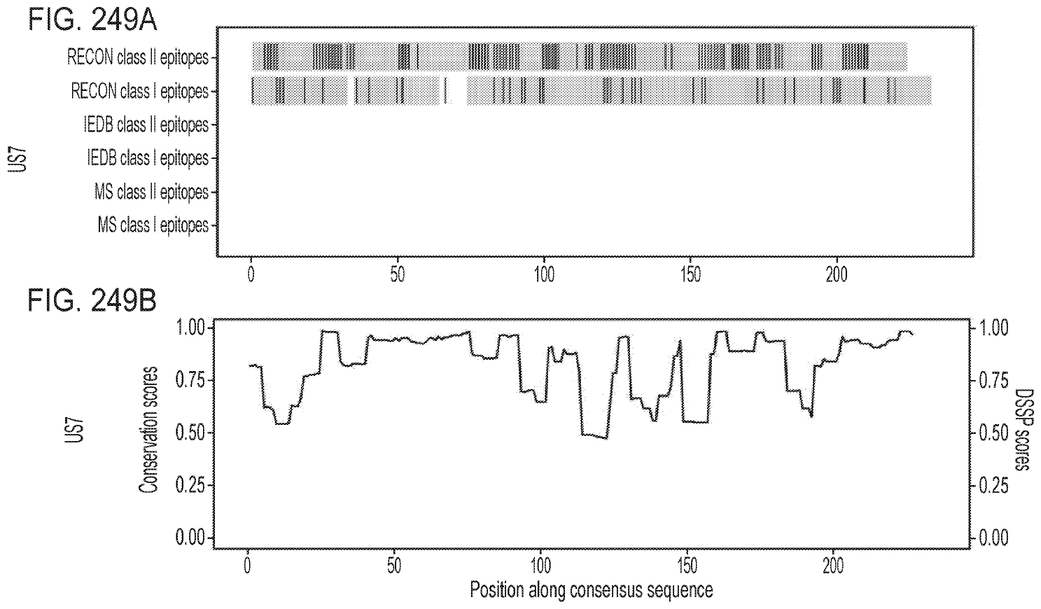

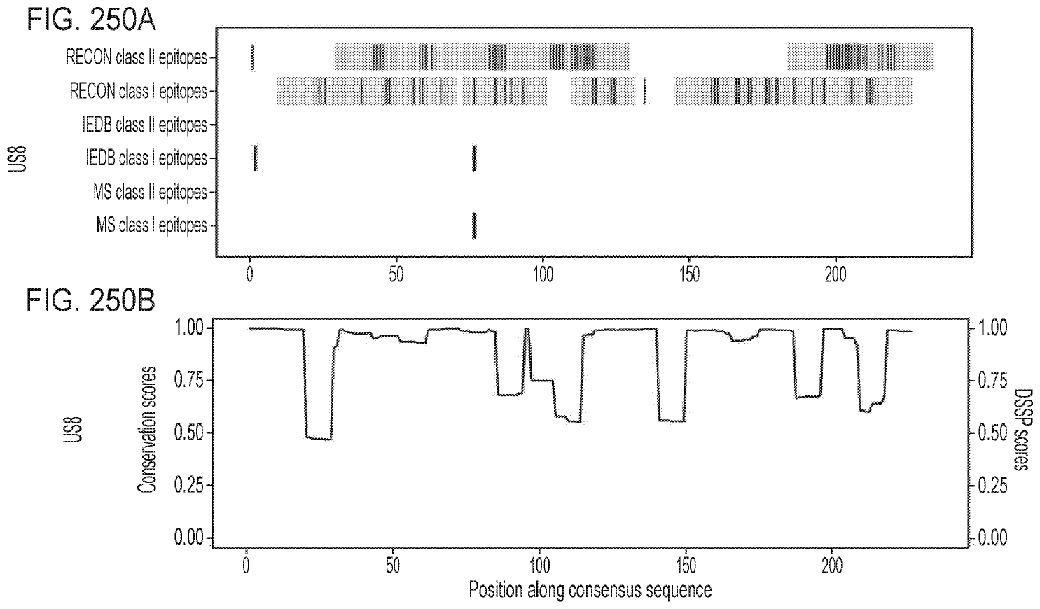

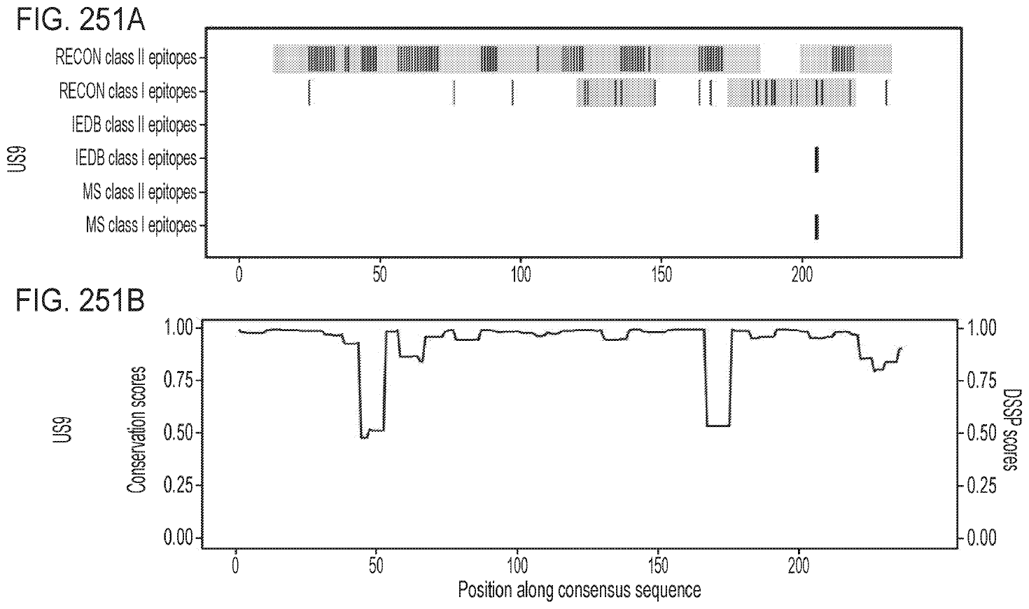

FIGS. 83A-251B shows cross-strain sequence conservation and epitopes of interest for an indicated CMV gene. Conservation is determined according to a sliding 9mer window as described above, where 0 indicates no conservation and 1 indicates that all strains are identical in the given region. Epitopes of interest include those highly predicted by RECON (percent rank <0.1 for HLA-I; percent rank <0.5 for HLA-II; for alleles common among individuals with European ancestry), identified by mass spectrometry-based HLA ligandomics, or otherwise identified in The Immune Epitope Database (iedb.org). It should be noted that positions are indexed with respect to the multiple sequence alignment, which contains gap characters.

### Example 18: Exemplary Methods for Determination of CMV “Block” Regions and String Design

The present Example provides exemplary methods for identifying “block” regions of a CMV polypeptide that may be useful for immunogenic compositions (e.g., vaccine compositions, and epitope binding agents, etc.). In some embodiments, “block” regions of a CMV polypeptide can be expressed by CMV during a latent phase of its life cycle and/or during an active infection. In some embodiments, such “block” regions can be or comprise CD4+ and/or CD8+ T cell epitopes. In some embodiments, such “block” regions can be or comprise B cell epitopes. In some embodiments, such “block” regions can be or comprise predominantly CD4+ and/or CD8+ T cell epitopes.

In some embodiments, one or more “block” regions identified in the present Example can be encoded by a polynucleotide. In some embodiments, such a polynucleotide can be a polyribonucleotide (e.g., in some embodiments mRNA). In some embodiments, a plurality of (including, e.g., at least two, at least three, at least four, at least five, at least six, at least seven, at least eight, at least nine, at least ten, or more) “block regions” identified in the present Example can be linked by one or more linkers. In some embodiments, such a polynucleotide can comprise a secretory signal sequence (e.g., in some embodiments at the N-terminal of the polynucleotide). In some embodiments, such a polynucleotide can comprise an MHC trafficking signal sequence (e.g., a MHC class I trafficking signal sequence; MITD sequence), for example, in some embodiments at the C-terminal of the polynucleotide.

For each CMV protein, a sliding window of 15 amino acid positions are applied from start to end over the multiple sequence alignment (MSA) profile over all the sequences belonging to a particular strain. The number of peptides highly scored by RECON (percent rank <0.1 for HLA-I; percent rank <0.5 for HLA-II) for alleles that are common in people of European ancestry (A*02:01, A*01:01, A*03:01, A*24:02, A*11:01, B*07:02, B*08:01, B*44:02, B*35:01, B*44:03, C*07:01, C*07:02, C*04:01, C*06:02, C*03:04, DRB1*07:01, DRB1*15:01, DRB1*03:01, DRB1*01:01, DRB1*04:01, DRB4*01:03, DRB3*01:01, DRB3*02:02, DRB5*01:01, DRB3*03:01, DPA1*01:03/DPB1*04:01, DPA1*01:03/DPB1*02:01, DPA1*01:03/DPB1*04:02, DPA1*01:03/DPB1*03:01, DPA1*02:01/DPB1*01:01, DQA1*01:02/DQB1*06:02, DQA1*05:01/DQB1*02:01, DQA1*02:01/DQB1*02:02, DQA1*05:05/DQB1*03:01, DQA1*01:01/DQB1*05:01; these allele selections could be revised to match the allele frequencies applicable in the region(s) of vaccine distribution) that map to each of these windows are counted. Sliding windows with at least 10 (HLA-I) or 50 (HLA-II) such peptides are considered as “dense epitope regions”. Dense epitope regions are determined for HLA-I and HLA-II peptides separately, and for each type of peptides, dense epitope regions are merged into continuous blocks. These continuous blocks are then filtered to only contain intervals where the corresponding protein conservation scores previously determined are higher than 0.9.

For each block identified from the above steps, the reference sequence for the corresponding virus is then used to determine sequence—sequences encompassing the block interval plus at least 10 amino acids (including, e.g., at least 11 amino acids, at least 12 amino acids, at least 13 amino acids, at least 14 amino acids, at least 15 amino acids, or more) downstream and upstream of the block interval are taken to form chunk sequences. In some embodiments, sequences encompassing the block interval plus 14 amino acids downstream and 15 amino acids upstream of the block interval are taken to form chunk sequences. The chunk sequences are filtered to contain only prioritized genes, and linked together using linkers (e.g., but not limited to linkers described herein such as, e.g., GSS linkers) with a secretary signal peptide domain (SP domain) in the N terminal and MITD domain in the C terminal. Exemplary sequences for the linker, N-terminal domain, and C-terminal domain are provided as follows:
SP domain: MKMRVMAPRTLILLLSGALALTETWAGS
MITD domain: IVGIVAGLAVLAVVVIGAVVATVMCRRKSSGGKGGSYSQAA SSDSAQGSDVSLTA
Start GSS linker (after SP domain): GGSGGGGSGG
Middle GSS linker (between blocks): GGSGGGGSGG
End GSS linker (before MITD): GGSLGGGGSG

The corresponding nucleotide sequence can be determined by reverse translation using the wildtype viral sequence, codon optimization, and/or some other codon selection scheme that optimizes one or more aspects of the resulting RNA string (e.g. stability, the lack of hairpins, etc.).

Tables 8A-8B contain CD8 and CD4-focused blocks, respectively, identified from the analyses as described above, as well as the corresponding string design. The columns “start” and “end” denote the starting and ending position of each identified region in the corresponding reference strain (TB40/E for CMV). The “gene” column provides the official gene symbol for each region, and the “seq” column provides the reference/exemplary strain sequence for that region. The final row of each table, with gene name “full string” gives the GSS linker-linked exemplary string design, with SP domain in the N terminal and MITD domain in the C terminal.

### Example 19: Exemplary Methods for Determination of HLA Binding Peptides (CMV)

All strain variants were downloaded from https://www.viprbrc.org/. All pairwise strain homologies were assessed by comparing the overlap of 9mer substrings of the constituent proteins; for a pair of strains, the number of 9mers present in both strains (the intersection) was divided by the number of 9mers present in at least one strain (the union) to derived a conservation score between 0 (no homology) and 1 (perfect homology). Outlier strains with poor homology to most other strains were excluded as a data cleaning procedure. The strain “TB40E” was chosen as an exemplar strain (Genbank Accession KF297339) for CMV and used for sequence indexing unless otherwise noted. The protein sequences of the remaining strains were dissolved into all possible substrings (“candidate HLA binding peptides”) of lengths 8-12. Candidate HLA binding peptides of lengths 8-12 were scored for binding potential to 105 HLA-I alleles using an algorithm called neonmhc1. Candidate HLA binding peptides of lengths 12-20 were scored for binding potential to 85 HLA-II alleles using an algorithm called neonmhc2. Neonmhcl is a proprietary algorithm based on proprietary data using methods similar to those published in Abelin et al. “Mass spectrometry profiling of HLA-associated peptidomes in mono-allelic cells enables more accurate epitope prediction”, Immunity (2017) 46(2):315-326, the entire contents of which are incorporated herein by reference for purposes described herein. Neonmhc2 is a proprietary algorithm based on proprietary data using methods similar to those published in Abelin et al., “Defining HLA-II ligand processing and binding rules with mass spectrometry enhances cancer epitope prediction” Immunity, (2019) 51(4):766-779.e17, the entire contents of which are incorporated herein by reference for purposes described herein. These two algorithms are part of a larger suite of proprietary analytical tools referred to as “RECON”; neonmhc1 predictions and neonmhc2 predictions are both occasionally referred to as “RECON” predictions herein. Neonmhc1 and neonmhc2 predictions are made on a continuous “percent rank” scale (Nielsen and Andreatta, “NetMHCpan-3.0; improved prediction of binding to MHC class I molecules integrating information from multiple receptor and peptide length datasets” Genome Medicine (2016) 8:33, the entire contents of which are incorporated herein by reference for purposes described herein) where smaller values indicate a higher likelihood of binding. Conservation was determined for each peptide by calculating the percentage of strains with at least one instance of the peptide within their proteome. Thus, a peptide with a conservation of 60% appears is encoded by 60% of the viral strains.

Table 10A contains peptides grouped according to likelihood of HLA-binding (per allele) and conservation predicted by neonmhc1, wherein sequences of such peptides have been submitted via DVD and are incorporated herein by reference in their entirety. “Very strong” indicates a percent rank binding score less than 0.1%. “Strong” indicates a percent rank binding score between 0.1% and 1%. “Moderate” indicates a percent rank binding score between 1% and 2%. Peptides with weaker predicted binding scores are not included in the table. “High conservation” indicates that the peptide is present in >90% of strains. “Moderate conservation” indicates that the peptide is present in 50%-90% of strains. “Poor conservation” indicates that the peptide is present in <50% of strains.

Table 10B contains peptides grouped according to likelihood of HLA-binding (per allele) and conservation predicted by neonmhc2, wherein sequences of such peptides have been submitted via DVD and are incorporated herein by reference in their entirety. “Very strong” indicates a percent rank binding score less than 0.5%. “Strong” indicates a percent rank binding score between 0.5% and 2%. “Moderate” indicates a percent rank binding score between 2% and 5%. Peptides with weaker predicted binding scores are not included in the table. “High conservation” indicates that the peptide is present in >90% of strains. “Moderate conservation” indicates that the peptide is present in 50%-90% of strains. “Poor conservation” indicates that the peptide is present in <50% of strains.

HLA binding peptides identified herein (e.g., HLA-I binding peptides and/or HLA-II binding peptides) can be delivered by immunogenic compositions described herein. In some embodiments, one or more HLA binding peptides (e.g., HLA-I binding peptides and/or HLA-II binding peptides) selected from Tables 10A-10B herein can be encoded by one or more polynucleotides (e.g., RNA) described herein. In some embodiments, a polynucleotide (e.g., RNA) described herein can encode a polyepitopic polypeptide comprising a plurality of (including, e.g., at least 2, at least 3, at least 4, at least 5, at least 6, at least 7, at least 8, at least 9, at least 10, at least 15, at least 20, at least 25, at least 30, or more) HLA binding peptides (e.g., HLA-I binding peptides and/or HLA-II binding peptides) selected from Tables 10A-10B herein.

### Example 20: Exemplary Methods for Determination of T Cell Epitopes (CMV)

In order to prioritize CD4 and CD8 epitopes for RNA string design for each virus, in some embodiments all known immunogenic and/or HLA-presented epitopes were first considered. Known immunogenic epitopes were curated from IEDB, only considering relevant T-cell epitopes, for example, in some embodiments only CD4 T-cell epitopes were considered for the CD4 shortlist. In some embodiments, B-cell epitopes and MHC ligands from IEDB were omitted. Known HLA-presented epitopes were curated from the HLA ligandomics datasets re-searched via mass spectroscopy (e.g., SpectraMill). Depending on the number of immunogenic and/or HLA-presented epitopes for the given virus and T-cell subset (CD4 or CD8), one of two procedures as described below were followed:
1) If the number of epitopes exceeded the maximum amount of epitopes desired (e.g., 70 for CD4, 100 for CD8), then epitopes were filtered out based on the following criteria, applied procedurally until less than the maximum amount of epitopes desired remained:
   Remove epitopes poorly conserved in viral strains (<50%) & with poor RECON prediction values for any prioritized HLA allele (>2%-rank for CD8, >5%-rank for CD4). A list of prioritized HLA alleles is provided below.
   Remove epitopes moderately conserved in viral strains (<90%)
   Remove epitopes with moderate RECON predictions for any prioritized HLA allele (>1%-rank for CD8, >2%-rank for CD4)
   Remove epitopes with strong RECON predictions for any prioritized HLA allele (>0.1%-rank for CD8, >0.5%-rank for CD4)
2) If the number of epitopes was less than the minimum amount of epitopes desired (e.g., 40 for CD4, 60 for CD8), then epitopes were supplemented using the following logic: for each prioritized HLA allele (e.g., as described below), epitopes were selected based on the strength of RECON predictions. Only epitopes which were highly conserved in viral strains (>90%) were considered. Epitopes were selected until the maximum number of epitopes desired would be exceeded while keeping the number of epitopes selected per allele equal.

HLA allele prioritization: The top 5 most frequent HLA alleles by US population (data from Gragert et al., “Six-locus high resolution HLA haplotype frequencies derived from mixed-resolution DNA typing for the entire US donor registry” Human Immunology (2013) 74(10): 1313-1320, the entire contents of which are incorporated herein by reference for the purposes described herein) were selected for this analysis. For Class I (CD8), this included the A, B, and C loci. For Class II (CD4), this included the DRB1, DRB345, DQ, and DP loci. If desirable, additional HLA alleles can be included for analysis.

The shortlisted epitopes were designed as RNA strings using approaches described below for HLA-I epitopes and HLA-II epitopes. For HLA-I epitopes, all pairwise epitope orderings were assessed for their likelihood of producing proteasomal cleavage events at the desired position. This was done using a proprietary cleavage predictor trained using proprietary data using methods similar to those presented in Abelin et al. “Mass spectrometry profiling of HLA-associated peptidomes in mono-allelic cells enables more accurate epitope prediction” Immunity (2017) 46(2): 315-326, the entire contents of which are incorporated herein by reference for the purposes described herein. Treating these pairwise scores as a directed graph wherein each epitope represents a node and each cleavage score represents an edge-wait, a heuristic approach was used (see, e.g., Hahsler M, “TSP: Traveling Salesperson Problem (TSP). R package version 1.1-10 (2020), which can be online accessible at https://CRAN.R-project.org/package=TSP; Hahsler M, “TSP-Infrastructure for the traveling salesperson problem” Journal of Statistical Software (2007) 23(2): 1-21, the entire contents each of which are incorporated herein by reference for the purposes described herein) to traverse the graph while maximizing on-target cleavability (noting that while the solution was referred as “optimal”, the algorithm is only a heuristic and not guaranteed to provide the globally optimal solution). Additionally or alternatively, in some embodiments, additional amino-acid linker sequences can be added between epitope pairs as needed to redress any adjacencies that that fail to meet suitable cleavage prediction scores. For HLA-II epitopes, the sequences were concatenated without linkers or special considerations for ordering. However, both approaches for HLA-I epitopes and HLA-II epitopes can be used to meet design objectives (such as reducing the presence of non-target junctional epitopes or achieving desired biophysical properties in the resultant polypeptide).

The top portion of Table 9A contains HLA-I epitopes (e.g., CD8 epitopes) shortlisted for inclusion in a string-of-epitopes vaccine, while the remaining portion of Table 9A contains HLA-II epitopes (e.g., CD4 epitopes) shortlisted for inclusion in a string-of-epitopes vaccine. Epitopes represent a mix of informatic predictions (per RECON) and sequences that have been defined as presented or immunogenic experimentally (per literature).

Table 9B in part shows the amino acid sequence corresponding to a candidate RNA string encoding a sequence of prioritized HLA-I epitopes. In some embodiments, the epitope sequences are ordered in a manner that maximizes the likelihood that the target epitopes will be processed and presented (see Methods as described above). In some embodiments, the sequence can additionally or alternatively be designed to minimize non-target junctional epitopes (for example, by choosing an epitope ordering that results epitope adjacencies unlikely to produce peptides that bind common HLAs), to achieve desired conformational properties in the resultant polypeptide (e.g., an enrichment of alpha helices, which could likewise be achieved by selecting an ordering with optimal epitope adjacencies), or some combination of the three objectives described herein. In some embodiments, one or more linker sequences can be additionally or alternatively used to achieve the above-described objectives. In some embodiments, the corresponding nucleotide sequence can be determined by reverse translation using the wildtype viral sequence, codon optimization, and/or some other codon selection scheme that optimizes one or more aspects of the resulting RNA string (e.g., but not limited to stability, the lack of hairpins, etc.).

Table 9B also shows the amino acid sequence corresponding to a candidate RNA string encoding a sequence of prioritized HLA-II epitopes. In some embodiments, by permuting the epitopes and/or adding one or more linkers, the sequence could be further optimized to achieve specific features (e.g., lack of non-target junctional epitopes and/or presence of desired polypeptide properties, as described herein). In some embodiments, the corresponding nucleotide sequence can be determined by reverse translation using the wildtype viral sequence, codon optimization, and/or some other codon selection scheme that optimizes one or more aspects of the resulting RNA string (e.g., but not limited to stability, the lack of hairpins, etc.).

### Example 21: Exemplary RNA Constructs Encoding CMV Antigens

The present example describes certain exemplary CMV antigens, and sequences encoding them, that may be utilized in certain embodiments of the present disclosure.

In some embodiments, an antigen delivered in accordance with the present disclosure is or comprises a CMV protein or an immunogenic portion thereof (e.g., comprising one or more epitopes thereof) as indicated in Table 6A or a CMV protein encoded by a gene listed in Tables 7A-7B or an immunogenic portion thereof (e.g., comprising one or more epitopes thereof). In some embodiments, an antigen delivered in accordance with the present disclosure is or comprises one or more regions of one or more CMV proteins, which regions each independently comprise one or more CD4+ epitopes and/or one or more CD8+ epitopes as indicated in Tables 8A-8B. In some embodiments, an antigen delivered in accordance with the present disclosure is or comprises a CMV peptide as indicated in Table 9A, Table 10A, or Table 10B. In some embodiments, such an antigen is delivered by administration of an RNA encoding such an antigen.

In some particular embodiments, an administered RNA has a structure:
Structure 1: m₂^{7,3′-O}Gppp(m₁^{2′-O})ApG-hAg-Kozak-SEC-Immunogen —FI-A30L70, wherein m₂^{7,3′-O}Gppp(m²′)ApG=5′ cap; hAg=5′ UTR human alpha-globin; SEC=signal peptide (SP); Immunogen=a nucleotide sequence comprising a sequence that encodes an antigen described herein; FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence.

In some embodiments, an administered RNA has a structure:
Structure 2: m₂^{7,3′-O}Gppp(m₁^{2′-O})ApG-hAg-Kozak-SEC-Immunogen-MITD-FI-A30L70, wherein m₂^{7,3′-O}Gppp(m₁^{2′-O})ApG=5′ cap; hAg=5′ UTR human alpha-globin; SEC=signal peptide (SP); Immunogen=a nucleotide sequence comprising a sequence that encodes one or more antigens described herein; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence.

In some embodiments, an Immunogen sequence encodes a plurality of immunogenic fragments (e.g., comprising epitopes) from an antigen. In some embodiments, an Immunogen sequence encodes a plurality of immunogenic fragments (e.g., epitopes) from two or more antigens. In some embodiments. In some embodiments, such immunogenic fragments are linked together to form an Immunogen sequence by linkers (e.g., in some embodiments a linker that is enriched in G and/or S amino acid residues). In some embodiments, a linker may be or comprise an amino acid sequence of GGSGGGGSGG. In some embodiments, a linker may be or comprise an amino acid sequence of GGSLGGGGSG.

### Example 22: Exemplary RNA Constructs Encoding Multiepitope CMV Antigens

The present example describes certain exemplary CMV multiepitope antigens, and sequences encoding them, that may be utilized in certain embodiments of the present disclosure.

### A) Exemplary Construct Encoding a CMV Multi-Epitope Polypeptide #1

Structure m₂^{7,3′-O}Gppp(m₁^{2′-O})ApG-hAg-Kozak-SEC-CD8 string-MITD-FI-A30L70, wherein m₂^{7,3′-O}Gppp(m₁^{2′-O})ApG=5′ cap; hAg=5′ UTR human alpha-globin; SEC=signal peptide (SP); CD8 string=a nucleotide sequence comprising a sequence that encodes a plurality of (e.g., two or more) CD8+ T cell epitopes described herein; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence. In some embodiments, the MITD element may be absent.

In some embodiments, a CD8 string may comprise at least 2 (including, e.g., at least 1, at least 2, at least 3, at least 4, at least 5, at least 10, at least 15, at least 20, or more) CD8+ T cell epitopes and/or HLA-I epitopes as listed in Table 9A or Table 10A. In some embodiments, a CD8 string may comprise an amino acid sequence as recited in Table 9B.

### B) Exemplary Construct Encoding a CMV Multi-Epitope Polypeptide #2

Structure m₂^{7,3′-O}Gppp(m₁^{2′-O})ApG-hAg-Kozak-SEC-CD4 string-MITD-FI-A30L70, wherein m₂^{7,3′-O}Gppp(m₁^{2′-O})ApG=5′ cap; hAg=5′ UTR human alpha-globin; SEC=signal peptide (SP); CD4 string=a nucleotide sequence comprising a sequence that encodes a plurality of (e.g., two or more) CD4+ T cell epitopes described herein; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence. In some embodiments, the MITD element may be absent.

In some embodiments, a CD4 string may comprise at least 2 (including, e.g., at least 1, at least 2, at least 3, at least 4, at least 5, at least 10, at least 15, at least 20, or more) CD4+ T cell epitopes and/or HLA-II epitopes as listed in Table 9A or Table 10B. In some embodiments, a CD4 string may comprise an amino acid sequence as recited in Table 9B.

### C) Exemplary Construct Encoding a CMV Multi-Epitope Polypeptide #3

Structure m₂^{7,3′-O}Gppp(m₁^{2′-O})ApG-hAg-Kozak-SEC-CD8 chunk sequence-MITD-FI-A30L70, wherein m₂^{7,3′-O}Gppp(m₁^{2′-O})ApG=5′ cap; hAg=5′ UTR human alpha-globin; SEC=signal peptide (SP); CD8 chunk sequence=a nucleotide sequence comprising a sequence that encodes one or more regions of one or more CMV polypeptides, wherein each region predominantly comprises CD8+ epitopes; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence. In some embodiments, the MITD element may be absent.

In some embodiments, a CD8 chunk sequence may comprise at least 2 (including, e.g., at least 1, at least 2, at least 3, at least 4, at least 5, at least 10, at least 15, at least 20, or more) immunogenic fragment(s) of polypeptide(s) encoded by gene(s) or peptide(s) as listed in Table 8A. In some embodiments, a sequence comprising SEC, CD8 chunk sequence, and MITD is represented by the sequence denoted as “full string” in Table 8A.

### D) Exemplary Construct Encoding a CMV Multi-Epitope Polypeptide #4

Structure m₂^{7,3′-O}Gppp(m^{2′-O})ApG-hAg-Kozak-SEC-CD4 chunk sequence-MITD-FI-A30L70, wherein m₂^{7,3′O}Gppp(m₁^{2′-O})ApG=5′ cap; hAg=5′ UTR human alpha-globin; SEC=signal peptide (SP); CD4 chunk sequence=a nucleotide sequence comprising a sequence that encodes one or more regions of one or more CMV polypeptides, wherein each region predominantly comprises CD4+ epitopes; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence. In some embodiments, the MITD element may be absent.

In some embodiments, a CD4 chunk sequence may comprise at least 2 (including, e.g., at least 1, at least 2, at least 3, at least 4, at least 5, at least 10, at least 15, at least 20, or more) immunogenic fragment(s) of polypeptide(s) encoded by gene(s) or peptide(s) as listed in Table 8B. In some embodiments, a sequence comprising SEC, CD4 chunk sequence, and MITD is represented by the sequence denoted as “full string” in Table 8B.

### Example 23: Exemplary CMV Peptide String Designs

The present Example exemplifies certain constructs (referred to herein as “strings”) of multiple CMV epitopes linked to one another and useful, for example, in immunogenic compositions (e.g., vaccine compositions) or otherwise as described herein.

Strings described in the present Example are designed to contain specific epitopes of CMV, each of which is disclosed herein and, e.g., is predicted and/or selected as described here, for example through use of an MHC-binding algorithm as described herein. The strings presented in the present Example are designed for therapeutic use in preventing and/or treating CMV infection (and in certain embodiments CMV latent infection) and can be administered as polynucleotide constructs, e.g., mRNA encapsulated in a lipid nanoparticle.

In some embodiments, strings exemplified herein are encoded in an RNA that includes a 5′-UTR and 3′-UTR. Epitopes are interconnected by peptide linkers, encoded by their respective polynucleotide sequences. In some embodiments, one or more linkers may have a specific cleavage site.

Exemplary amino acid sequences for exemplary strings denoted as “full_string” in Tables 8A-8B, each of which includes a SP domain, GS-enriched linkers, and a MITD domain. Table 9B shows additional exemplary amino acid sequences for exemplary CD8 string and CD4 strings. In some embodiments, polynucleotide sequences are codon optimized (e.g., for efficient translation in humans).

### Example 24: Exemplary CMV Antigen Identification, Selection and/or Characterization

The present Example describes identification, selection and/or characterization of certain CMV protein sequences useful as or in (i.e., as part of) antigens as described herein.

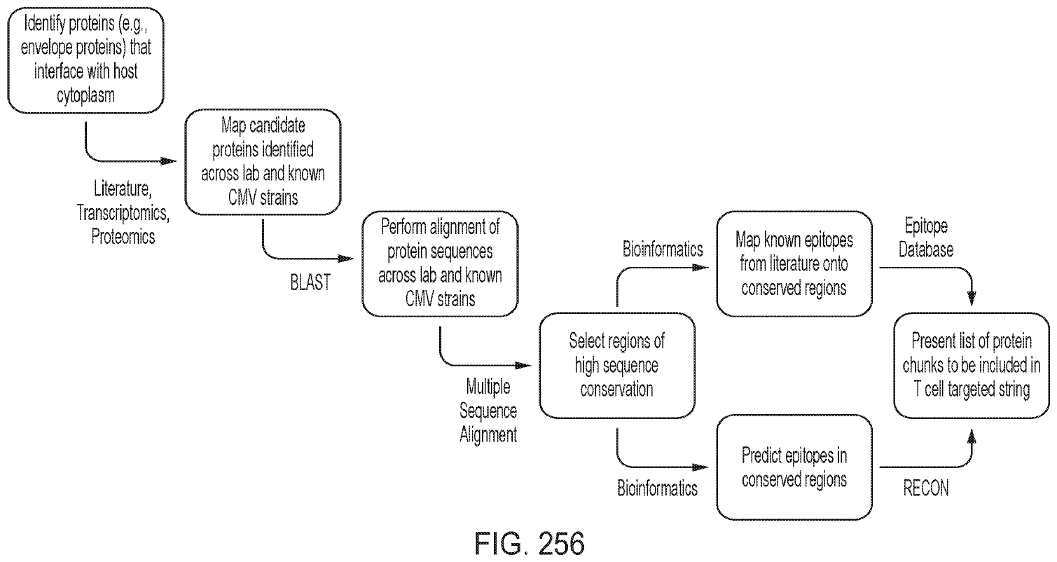

FIG. 256 presents a flow diagram of a process used to identify, characterize, and/or select certain CMV protein sequences (e.g., particular variants) and/or fragments or epitopes thereof, that may be particularly useful in the practice of the present invention. In some embodiments, a utilized antigen is or comprises a CMV protein, or fragment or epitope thereof, as identified or characterized as described in the present Example.

As depicted, proteins expressed prior to cell infiltration/infection and include one or more portions expected or known to interface with host cytoplasm are identified, for example by literature review, considering transcriptomic (i.e., RNA expression levels) and/or proteomic (i.e., expressed protein levels) data. Degree of conservation of candidate proteins across relevant CMV strains (e.g., in relevant geographic region) is considered.

Various lab and field isolate strains can be considered for assessing conserved proteins and T cell epitopes. In some embodiments, strain TB40/E was considered for assessing conserved proteins and T epitopes.

Immunogenicity of conserved proteins was also considered, for example by review of literature and/or application of predictive algorithms as described herein.

In accordance with the present example, in some embodiments, each of the CMV proteins encoded by genes shown in Tables 7A-7B and combinations thereof were identified as of particular interest. In some embodiments, one or more of these proteins, or one or more fragments or epitopes thereof is used as or included in an antigen. In some embodiments, a peptide or protein is encoded by an UL44 gene, an UL86 gene, or combinations thereof. In some embodiments, a peptide or protein is encoded by a gene selected from: UL32, UL44, UL55, UL75, UL83, and combinations thereof.

In some embodiments, an antigen may be or comprise one or more, and specifically may comprise a plurality, of distinct portions (e.g., epitope-containing fragments) of one or more of these proteins, for example in a string construct as described herein. In some embodiments, exemplary CD8 string candidates were designed utilizing one or more epitope-containing fragments of a plurality of proteins encoded by the following genes: UL122, UL123, UL32, UL55, and UL75; representative of such epitope-containing fragments are shown in Table 8A, and a corresponding exemplary string candidate is shown in Table 8A (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA).

In some embodiments, exemplary CD4 string candidates were designed utilizing one or more epitope-containing fragments of a plurality of proteins encoded by the following genes: UL122, UL123, UL32, UL44, UL55, UL75, and UL83; representative of such epitope-containing fragments are shown in Table 8B, and a corresponding exemplary string candidate is shown in Table 8B (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA).

In some embodiments, a secretory signal (“See”) or a signal peptide (SP) domain present in exemplary string candidates described herein (e.g., a CD8 string and/or a CD4 string as described herein) may be that from HSV-2 gD SP MGRLTSGVGTAALLVVAVGLRVVCA; in alternative embodiments, a different secretory signal, e.g., from HSV-1 gD SP, is used. In some embodiments, a signal peptide may be or comprise an IgE signal peptide. In some embodiments, a signal peptide may be or comprise an IgE HC (Ig heavy chain epsilon-1) signal peptide. In some embodiments, a signal peptide that may be useful in accordance with the present disclosure may comprise one of the following sequences:
MDSKGSSQKGSRLLLLLVVSNLLLPQGVVG;
MDWTWILFLVAAATRVHS;
METPAQLLFLLLLWLPDTTG;
MLGSNSGQRVVFTILLLLVAPAYS (Japanese encephalitis PRM signal sequence);
MKCLLYLAFLFIGVNCA (VSVg protein signal sequence);
MWLVSLAIVTACAGA (Japanese encephalitis JEV signal sequence); or
MFVFLVLLPLVSSQC.

In some embodiments, certain chunk boundary considerations are incorporated into the string constructs, for example establishing chunk boundaries to minimize presence of sequences (e.g., epitopes) that may overlap with the human proteome.

### Example 25: Exemplary Vaccine Composition Delivery at Least One CMV Antigen

The present Example describes certain exemplary vaccine compositions:

In some embodiments, a provided vaccine candidate will contain at least 2 RNAs, at least one of which encodes a CMV antigen described herein (e.g., a full length CMV protein or one or more fragments or epitopes thereof, such as a string construct described herein), and optionally at least one of which encodes at least one other conserved CMV protein (or fragment(s) or epitope(s) thereof, such as in a string construct as described herein; in some embodiments such a string construct may include fragments or epitopes from two or more different CMV proteins).

In some embodiments, two or more (e.g., 3, for example 2 of which are/encode CMV antigen string constructs and one of which is/encodes a string construct of a plurality of CD8 and/or CD4 epitopes from other conserved CMV proteins) are formulated together in a single LNP formulation; in other embodiments, individual RNAs may be separately formulated in (the same or different) LNP formulations and such may be mixed together (e.g., in a 1:1 ratio of each RNA, or alternatively in a 1:1 ratio of CMV-antigen-encoding-RNA to “other” RNA so that, for example, for a composition comprising 2 CMV-antigen-encoding RNAs and one other RNA, the ratios would be 0.5:0.5:1).

### Example 26: Exemplary Pre-Clinical Assessment for CMV RNA Vaccine Compositions

The present Example describes certain pre-clinical assessments that may be performed of certain RNA vaccine compositions described herein for treatment and/or prevention of VZV infection and in certain particular embodiments for treatment and/or prevention of CMV latent infection.

In some embodiments, one vaccine candidate is assessed. In some embodiments, more than one different vaccine candidate may be assessed. In some such embodiments, different candidates may vary, for example, in:
RNA platform (e.g., unmodified RNA, modified RNA, saRNA);
Encoded antigen(s);
Number of RNAs;
Non-coding elements of RNA construct (e.g., cap and/or cap-adjacent sequences, 5′-UTR, 3′-UTR, and/or PolyA tail); and/or
Lipid composition of LNP.

In some embodiments, pre-clinical assessment of certain RNA vaccine compositions (e.g., LNP formulated mRNA-based CMV vaccines) comprises one or more of assessment in challenge experiments, assessment of level of protection, assessment of immunogenicity, and/or assessment of functional antibody responses.

LNP formulated mRNA-based CMV vaccines are tested in a challenge model. Non-human primate models, such as Rhesus macaques and Cynomolgus monkey, and/or rodent models, such as C57/B16 mice, Balb/c mice or NODscidIL2Rγnull mice; and/or guinea pig models, inoculated with CMV, are administered a first vaccination and can be administered an additional vaccination (e.g., 1, 2, 3, 4, 5, 6, 7, 8, 9, or 10 additional vaccinations) following the first vaccination. Wherein more than one vaccination is administered, the vaccinations are administered at an interval of 1, 2, 3, 4, 5, 6, 7, or 8 week intervals). Following vaccination, animals are challenged by CMV. Alternatively or additionally, animals are challenged by intravenous, subcutaneous, and/or intramuscular injection of virus-infected lymphocytes. Lymphocytes can be infected with any suitable strain of CMV. Animals are then evaluated for reduced infection of neurons. In some embodiments, an animal model is challenged in a plurality of instances (e.g., before first vaccination and/or wherein additional vaccinations are administered, at any time point between or after vaccinations). Following challenge, animals subjected to the study may be assessed according to any method known in the art, including, for example, serology assessment, immunogenicity, level of protection, etc.

In some embodiments, serum antibody characterization and/or serum transfer experiments (e.g., from one vaccinated species to a different non-vaccinated species, e.g., from vaccinated non-human primate to non-vaccinated mouse) are conducted (e.g., to assess protective antibody response).

In some embodiments, certain RNA vaccine compositions of the present disclosure are assessed for level of protection. Level of protection can be assessed according to any suitable method known in the art.

In some embodiments, certain RNA vaccine compositions of the present disclosure are assessed for immunogenicity. For example, ELISA can be used to determine IgG specific (and subclasses thereof) titers and/or avidity of antibodies generated in response to certain RNA vaccine compositions of the present disclosure to CMV antigens. In some embodiments, serum antibody titers against CMV glycoprotein (e.g., gH and/or gL glycoprotein, etc.) is determined by ELISA using standard methods. In some embodiments, for example, ELISpot (e.g., for CD8+, CD4+ T cells and/or IFNγ) and assessment of pro-inflammatory cytokine responses with splenocytes from immunized and/or challenged animal models and peptide pools derived from vaccine targets can also be assessed. In some embodiments, for example, phenotyping of immune responses (e.g., by flow cytometry) are assessed. In some embodiments, for example, T cell depletion and/or protection assays are conducted to assess immunogenicity (e.g., according to any suitable known method in the art).

In some embodiments, one or more functional responses of antibodies generated in response to certain RNA vaccine compositions of the present disclosure are assessed. Functional antibody responses can be assessed, for example, using a CMV neutralization assay. In some embodiments, a CMV in vitro neutralization assay is performed to evaluate one or more anti-CMV glycoprotein (e.g., gH and/or gL glycoprotein, etc.) antibodies in neutralizing CMV. For example, anti-CMV glycoprotein antibodies are obtained by collecting the sera of animals (e.g., mice) vaccinated with CMV mRNA vaccines. CMV virus are added to the diluted sera and neutralization is allowed to continue for 1 hour at room temperature. 3T3 cells are seeded in 96-wells one day before and the virus/serum mixtures are added to 3T3 monolayers. The cells are fixed on the next day and CMV-specific staining is performed. The plates are scanned and analyzed. A neutralization titer is expressed as the highest serum dilution required to achieve a 50% reduction in the number of plaques.

In some embodiments, functional antibody responses can be assessed, for example, using passive transfer studies of sera from immunized animals to naïve animals that are challenged and assessing level of protection.

### Example 27: Exemplary Characterization Studies of CMV Vaccine Candidates or Vaccine Compositions

The present Example describes certain potential characterization studies that may be utilized, for example, to identify, select, and/or characterize vaccine candidates or vaccine compositions (e.g., manufacturing batches thereof), or components thereof as described herein.

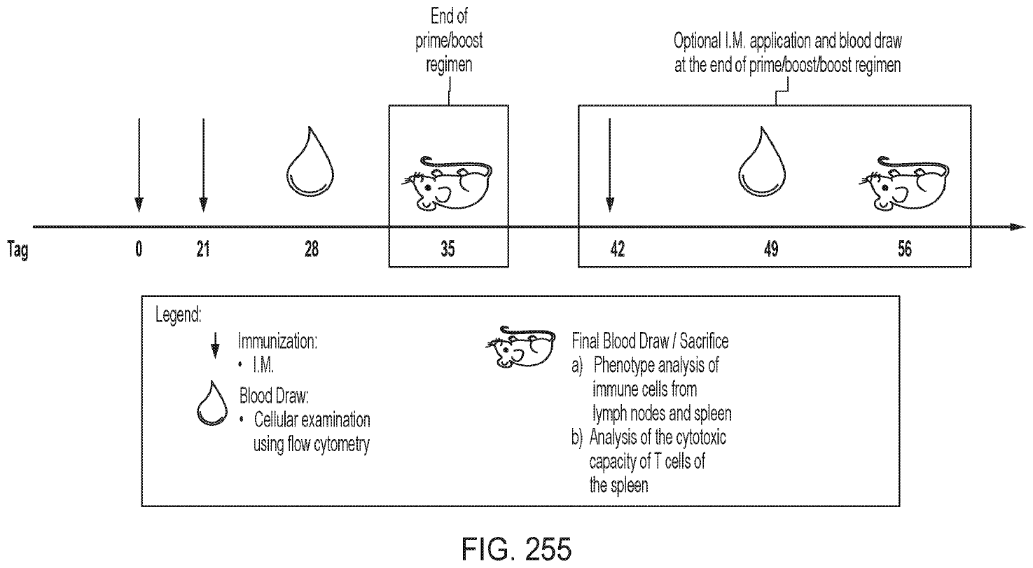

FIG. 255 presents a potential immunization protocol that can be utilized to assess ability of a vaccine candidate that comprises or delivers an antigen(s) as described herein to induce B- and/or T-cells, e.g., after intramuscular immunization, directed to the antigen(s) and/or epitope(s) thereof. In some embodiments, level and/or diversity of response is determined. In some embodiments, presence and/or level of neutralizing antibodies is/are determined. In some embodiments, protection of the immunized subject from challenge with CMV is assessed.

Alternatively or additionally, in some embodiments, one or more in vitro assessments may be performed, for example:
(1) in vitro expression of an antigen encoded by an RNA included in a vaccine composition; and/or
(2) in vitro potency of antigen expressed from an RNA included in a vaccine composition as described herein.

### Example 28: Exemplary Clinical Studies of CMV RNA Vaccine Compositions

The present Example describes certain clinical assessments that may be performed of certain RNA vaccine compositions described herein for treatment and/or prevention of VZV infection and in certain particular embodiments for treatment and/or prevention of VZV latent infection.

In some embodiments, more than one different vaccine candidate may be assessed. In some such embodiments, different candidates may vary, for example in:
(1) RNA platform (e.g., unmodified RNA, nucleoside-modified RNA, self-amplifying RNA (saRNA), trans-amplifying RNA);
(2) encoded antigen—e.g.,
   which CMV protein(s) utilized
   full length protein antigen vs fragment vs plurality of fragments vs fusion with one or more heterologous sequences (e.g., membrane tether, secretion, linker(s))
   epitopes from different (and/or multiple) phases of CMV life cycle
(3) number of RNAs
(4) non-coding elements of RNA construct
   cap and/or cap-adjacent sequences
   5′ UTR
   3′ UTR
   polyA tail
(4) lipid composition of LNP.

In one particular embodiment, up to three candidate vaccines that have only 1 mRNA encoding for a CMV glycoprotein (e.g., CMV gH, gL, gB, gO, gN, gM, g24 or any glycoprotein described in Table 6A or 6B) or a CMV protein or variant are evaluated and/or up to three candidates that contain 2 mRNAs, one encoding for a CMV glycoprotein (e.g., CMV gH, gL, gB, gO, gN, gM, g24 or any glycoprotein described in Table 6A or 6B) or a CMV protein or variants and another one encoding for CD8 and/or CD4 epitopes from conserved antigens (and optionally considering conserved T-cell epitopes from various stages of the CMV life cycle) are evaluated. In this particular exemplary embodiment, vaccine candidates may be evaluated by intramuscular administration, for example, based on a dose-escalation scheme.

### Example 29: Exemplary Methods for Norovirus Clade Identification

The present Example provides exemplary methods for identifying norovirus clades.

All strain variants of norovirus were downloaded from https://www.viprbrc.org/. All pairwise strain homologies were assessed by comparing the overlap of 9mer substrings of the constituent proteins; for a pair of strains, the number of 9mers present in both strains (the intersection) was divided by the number of 9mers present in at least one strain (the union) to derived a conservation score between 0 (no homology) and 1 (perfect homology). These pairwise similarities were used to inform a hierarchical cluster analysis that grouped strains according to their sequence similarity; these groups are referred to as “clades”. The annotations of the individual strains per clade were used to define labels for the clades, which include GI, GII.P2, GII.P4, GII.P7, GII.P12, GII.P16, GII.P17, and GIX. A small number of strains (<5%) were not assigned to any clade.

For each clade an exemplar sequence was selected. The exemplar was selected based on having a complete and clear gene annotation and having strong (e.g., better than average) homology (per the 9mer overlap approach described above) to the other strains in the clade.

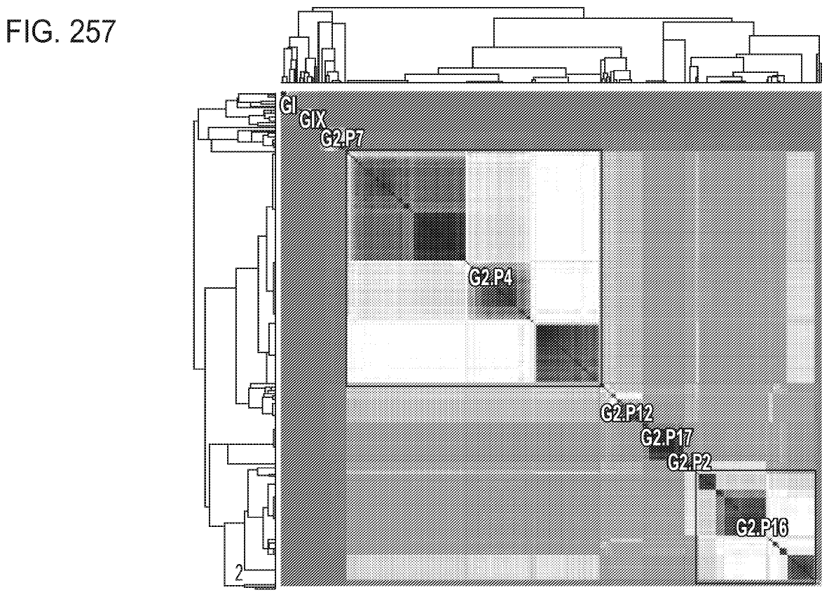

FIG. 257 is a visual representation of norovirus strains grouped by their sequence similarity. Each cell in the square gird represents the sequence similarity of a pair of strains according to a sliding color scale in which blue represents poor homology, white indicates moderate homology, and red indicates strong homology. The ordering of strains is identical for both rows and columns and is permuted such that clusters of similar sequences are evident. Dendrograms likewise display the presence of related strain groupings.

Table 11 contains protein sequences of exemplar sequences selected for each norovirus clade. Fasta names are in the format of clade I strain name I gene ID for each gene in each exemplar strain of Norovirus.

### Example 30: Exemplary Datasets for Norovirus (NoV) and Methods for IEDB Data Curation

The present Example provides exemplary datasets curated for identification of regions and/or epitopes of Norovirus (NoV) antigens that may be useful for immunogenic compositions (e.g., vaccine compositions, and epitope binding agents, etc.). In some embodiments, such regions and/or epitopes of NoV antigens can be expressed by NoV during a latent phase of its life cycle and/or during an active infection. In some embodiments, such regions and/or epitopes of NoV antigens can be or comprise T cell epitopes. In some embodiments, such regions and/or epitopes of NoV antigens can be or comprise B cell epitopes. In some embodiments, such regions and/or epitopes of NoV antigens can be or comprise predominantly T cell epitopes.

### Exemplary Datasets Curated for Norovirus (“NoV”)

Proteomics data for whole-cell lysate and M7GFP conditions was obtained from Supplementary tables 2-3 of Emmott et al. “Norovirus-Mediated Modification of the Translational Landscape via Virus and Host-Induced Cleavage of Translation Initiation Factors.” Mol Cell Proteomics. 2017; 16 (4 suppl 1): S215-S229. doi:10.1074/mcp.M116.062448, the entire contents of which are incorporated herein by reference for the purposes described herein.

Proteomics data for 6 and 10 hours post-infection with NoV was obtained from supplementary files in the PRIDE database. Brocard et al. “Murine Norovirus infection results in anti-inflammatory response downstream of amino acid depletion in macrophages.” [published online ahead of print, 2021 Aug. 4]. J Virol. 2021; JVI0113421, doi:10.1128/JVI.01134-21, the entire contents of which are incorporated herein by reference for the purposes described herein.

Pools of peptides (15mers) across all ORFs were evaluated for immunogenicity in 3 healthy donors, with each peptide represented in 3 different pools. 9- or 10-mers were predicted from candidate 15-mers using IEDB and NetMHCPan. Predicted peptides were then evaluated via multimer staining. Pattekar et al. “Norovirus-Specific CD8+ T Cell Responses in Human Blood and Tissues.” Cell Mol Gastroenterol Hepatol. 2021; 11(5):1267-1289. doi:10.1016/j.jcmgh.2020.12.012, the entire contents of which are incorporated herein by reference for the purposes described herein.

### IEDB Data Curation Process:

MHC ligands, T-cell epitopes, and B-cell epitopes were collected from IEDB from each virus, for example, including the downloadable datasets mhe_ligand_full.csv, tcell_full_v3.csv, and bcell_fullv3.csv, respectively. Filtering and data cleaning was performed to ensure these data only contained relevant epitopes, e.g. negative assay results and non-human viral strains were omitted. In order to summarize the number of unique epitope sequences in the data in an accurate, non-redundant manner, epitopes that were reported in unbiased studies (listed below) were removed. Lastly, the remaining epitopes were classified into six categories indicating the assay used and relevant MHC class, if applicable. T-cell epitopes that were not specified to be CD4+ or CD8+ specific were categorized as “Ambiguous T-cell epitopes”.

### Select Unbiased Studies Include:

Pattekar et al. ‘Norovirus-Specific CD8+ T Cell Responses in Human Blood and Tissues.” Cell Mol Gastroenterol Hepatol. 2021; 11(5):1267-1289. doi:10.1016/j.jcmgh.2020.12.012, the entire contents of which are incorporated herein by reference for the purposes described herein.

Tables 12A-12D present relevant gene-level data curated and/or re-analyzed from literature publications including the datasets described above. Such data include, for example, information relating to proteomics and immunogenicity (e.g., T cell epitopes). The tables also identify genes that can be useful for a vaccine described herein per systematic analysis and ranking process based on the exemplary curated data noted above.

### Example 31: Exemplary Methods for Assessing Suitability of NoV Genes as Targets for CD8+ and CD4+ T Cell Responses

The present Example provides exemplary methods for identifying NoV genes as targets for CD8+ and/or CD4+ T cell responses. In some embodiments, NoV genes identified as targets for CD8+ and/or CD4+ T cell responses may be useful for immunogenic compositions (e.g., vaccine compositions, and epitope binding agents, etc.). In some embodiments, regions and/or epitopes encoded by NoV genes identified using the methods described herein can be expressed by NoV during a latent phase of its life cycle and/or during an active infection. In some embodiments, such regions and/or epitopes of NoV antigens can be or comprise CD4+ and/or CD8+ T cell epitopes. In some embodiments, such regions and/or epitopes of NoV antigens can be or comprise predominantly CD4+ and/or CD8+ T cell epitopes.

Various lines of evidence were considered to assess the suitability of NoV genes as targets for CD8+ and CD4+ T cell responses. Depending on availability and applicability, RNA expression, protein expression, HLA ligandomics, and/or immunogenicity data from literature publications including the datasets as described in Example 30 were considered. In some embodiments, immunogenicity data was considered to be of higher importance if it was derived from an “unbiased study” (one which assessed genes across the whole viral proteome) than if it was derived from a “biased study” (one which only assessed immunogenicity for a single gene or a subset of genes in the viral proteome). Tables 13A-13B outline a step-wise process by which genes were tiered and/or eliminated from the final roster of selections. Lowest tier number (e.g., “Tier 1”) indicates highest importance. The “Description” column indicates the overall concept of the selection step; whereas the column “Selection Criteria” indicates the specific rule or thresholds used. The column “Genes” lists the genes that correspond to the given priority tier.

### Example 32: Exemplary Methods for NoV Multiple Sequence Alignments

For each protein, strain variants of a given clade were aligned into a global sequence alignment, for example, using MAFFT (default parameters; https://www.ebi.ac.uk/Tools/msa/mafft/). Cross-strain conservation across each protein sequence was determined, for example, by applying a sliding 9mer window and calculating the frequency of the most frequent 9mer at each position. The sequence was further annotated by indicating the starting positions of peptides highly scored by T cell epitope prediction tools from the bioinformatics pipeline RECON® (Real-time Epitope Computation for Oncology) (Abelin et al. Immunity (2017) 21:46:315-26; Abelin et al., Immunity (2019) 15; 51:766-779.e17) (percent rank <0.1 for HLA-I; percent rank <0.5 for HLA-II) for alleles that are common in people of European ancestry (A*02:01, A*01:01, A*03:01, A*24:02, A*11:01, B*07:02, B*08:01, B*44:02, B*35:01, B*44:03, C*07:01, C*07:02, C*04:01, C*06:02, C*03:04, DRB1*07:01, DRB1*15:01, DRB1*03:01, DRB1*01:01, DRB1*04:01, DRB4*01:03, DRB3*01:01, DRB3*02:02, DRB5*01:01, DRB3*03:01, DPA1*01:03/DPB1*04:01, DPA1*01:03/DPB1*02:01, DPA1*01:03/DPB1*04:02, DPA1*01:03/DPB1*03:01, DPA1*02:01/DPB1*01:01, DQA1*01:02/DQB1*06:02, DQA1*05:01/DQB1*02:01, DQA1*02:01/DQB1*02:02, DQA1*05:05/DQB1*03:01, DQA1*01:01/DQB1*05:01; in some embodiments, such allele selections can be changed to match the allele frequencies applicable in the region(s) of vaccine distribution). Other peptides of interest (those identified by HLA ligandomics studies or otherwise reported in The Immune Epitope Database (iedb.org)) were also positionally located in the global sequence alignments. The entire contents of the aforementioned documents are incorporated herein by reference for the purposes described herein.

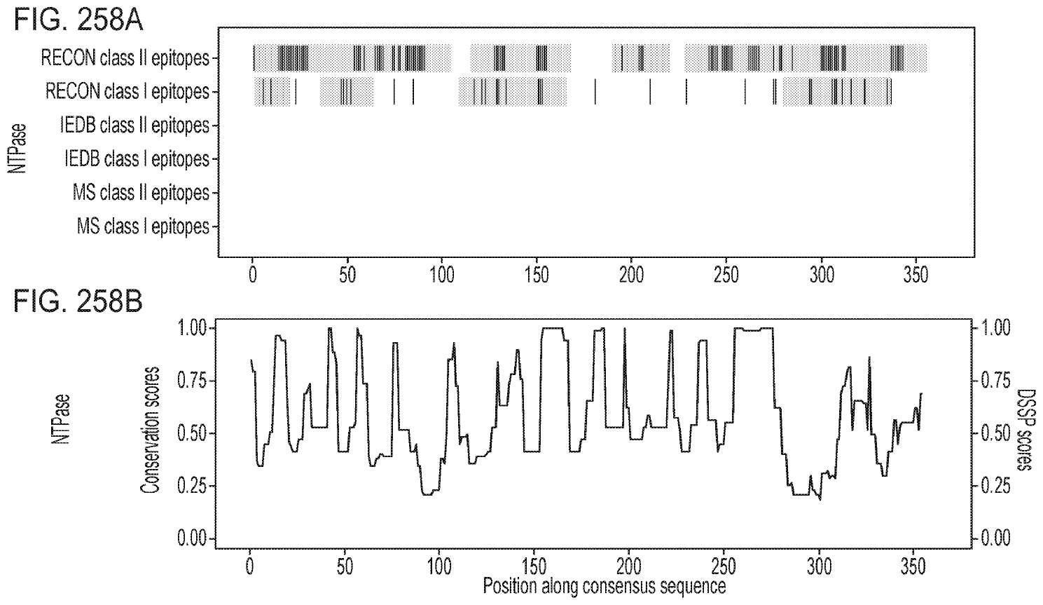

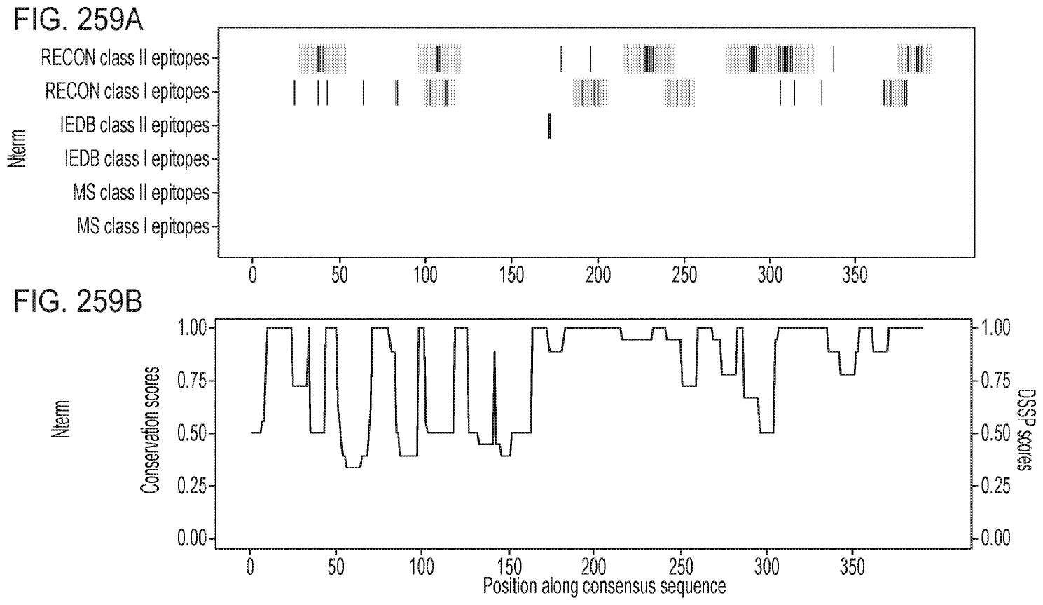

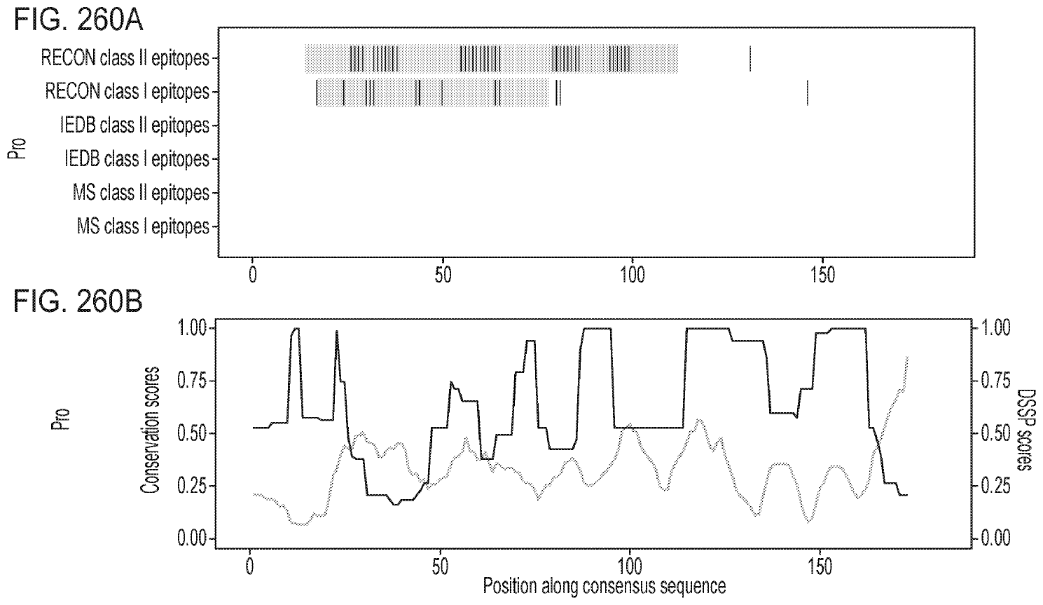

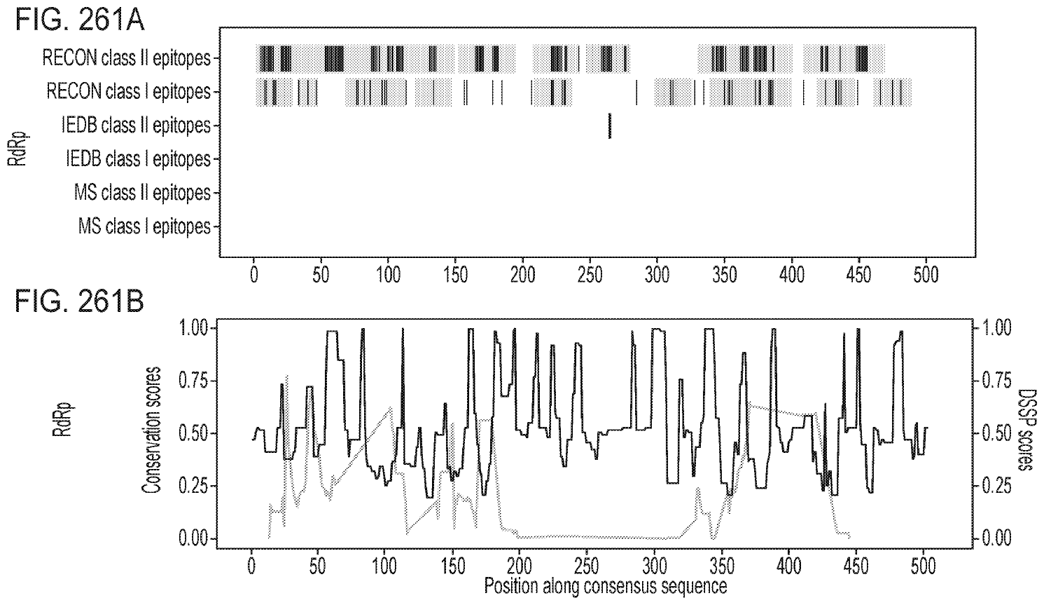

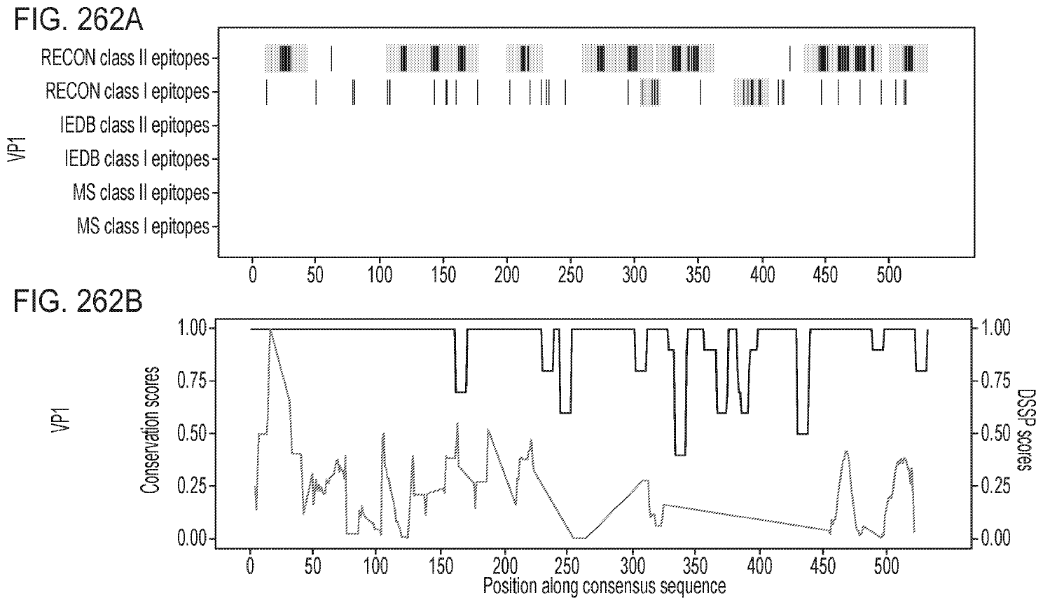

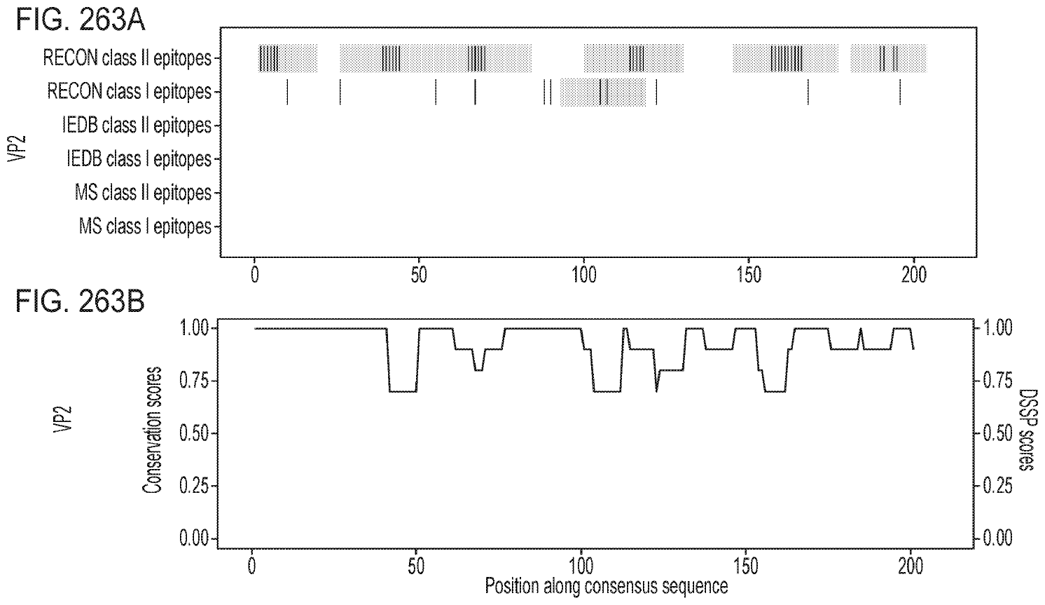

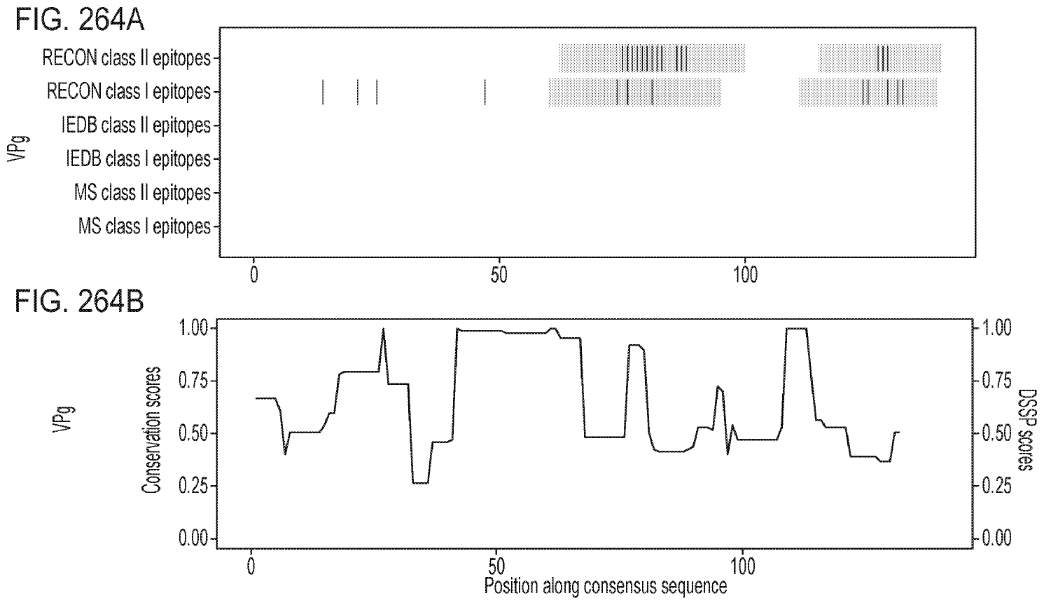

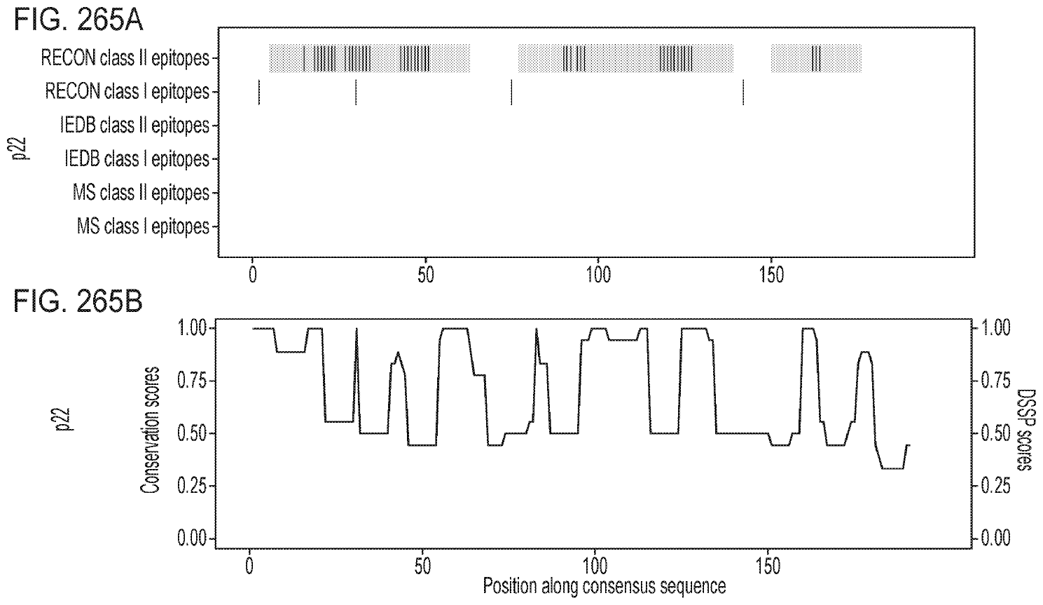

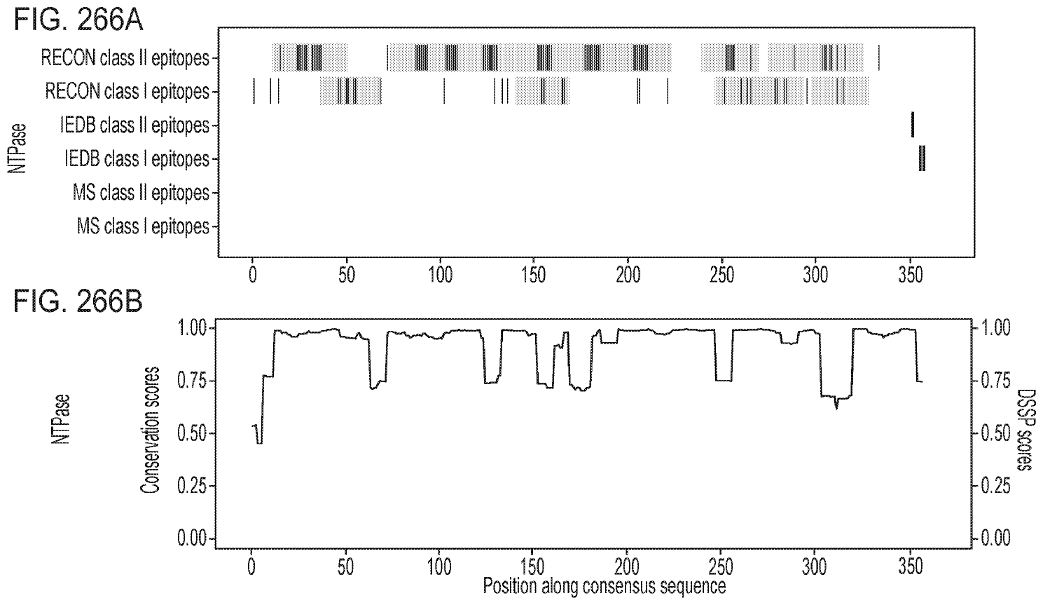

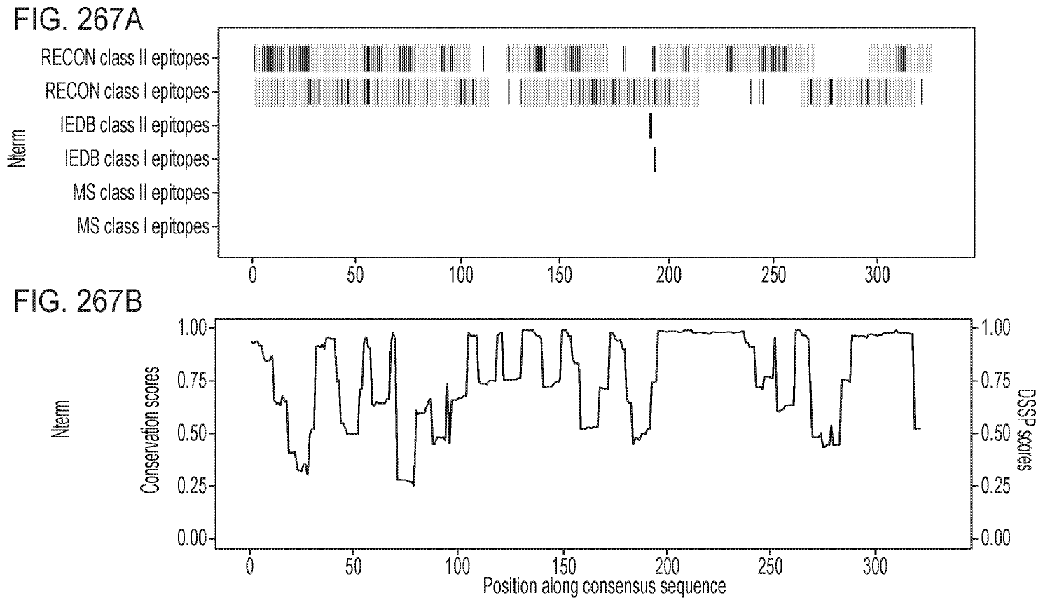

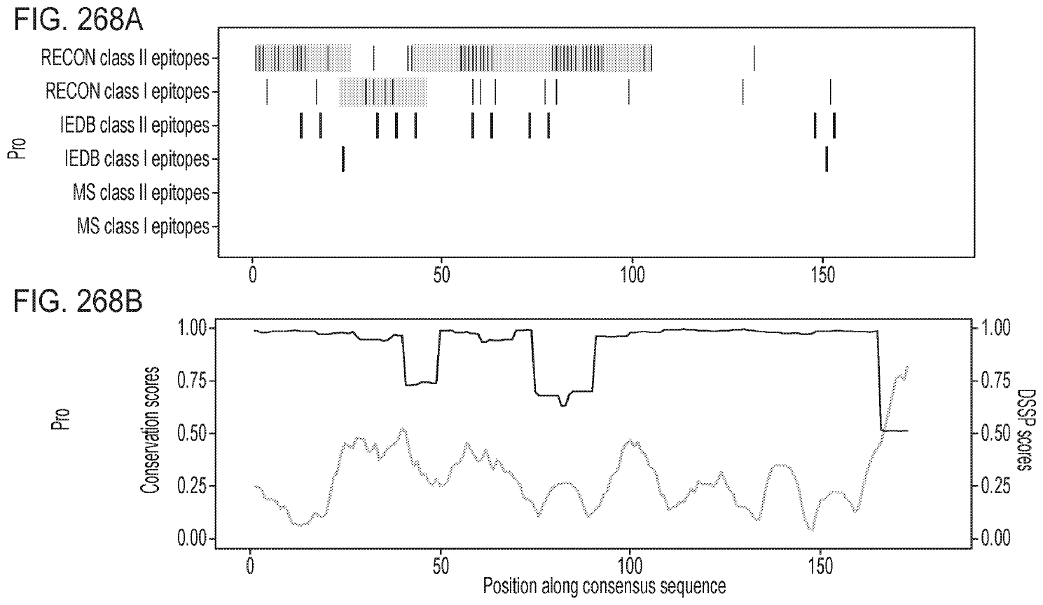

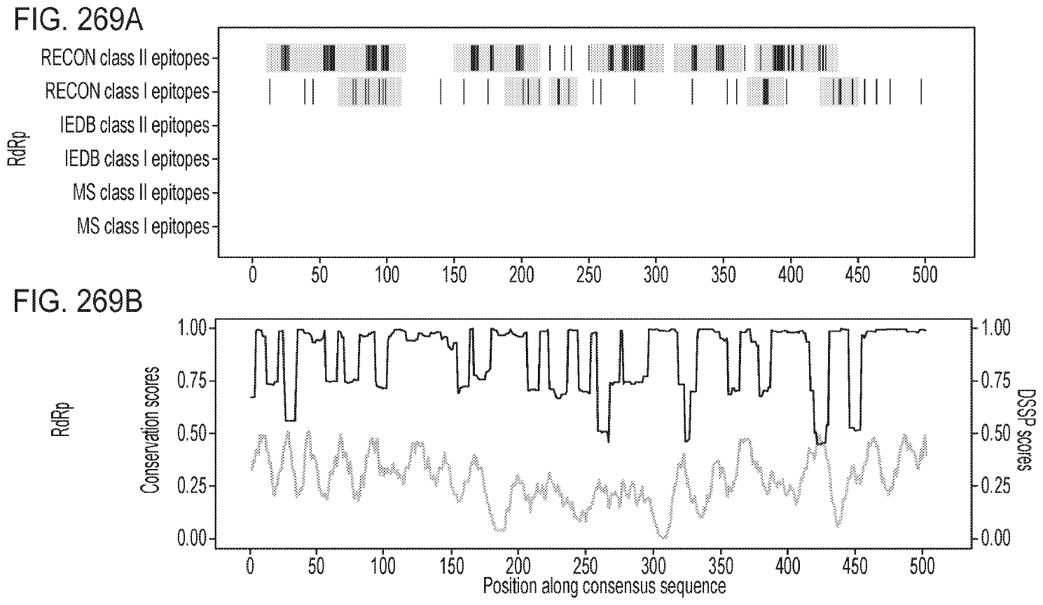

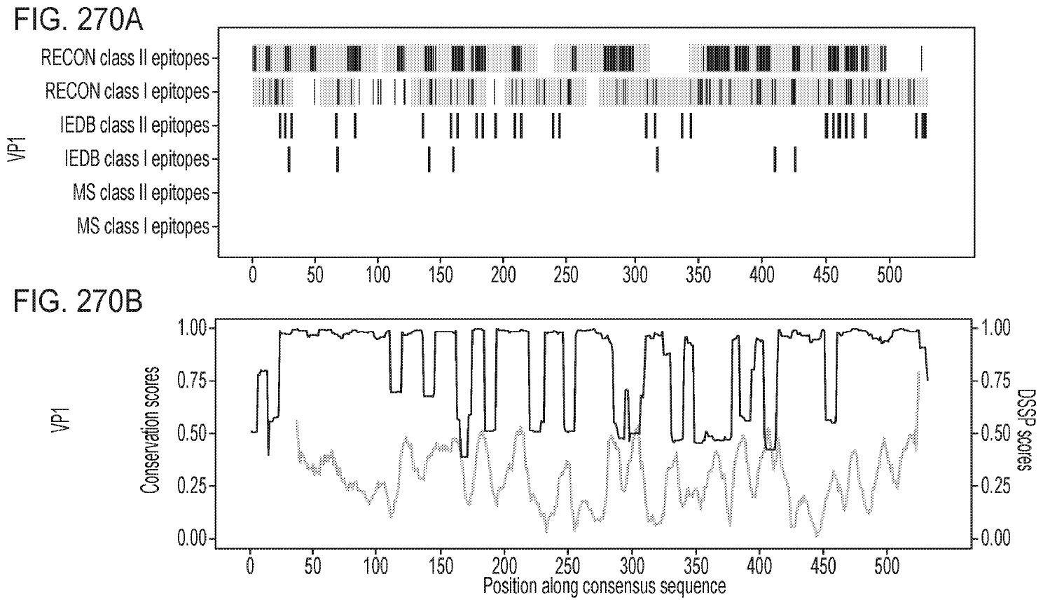

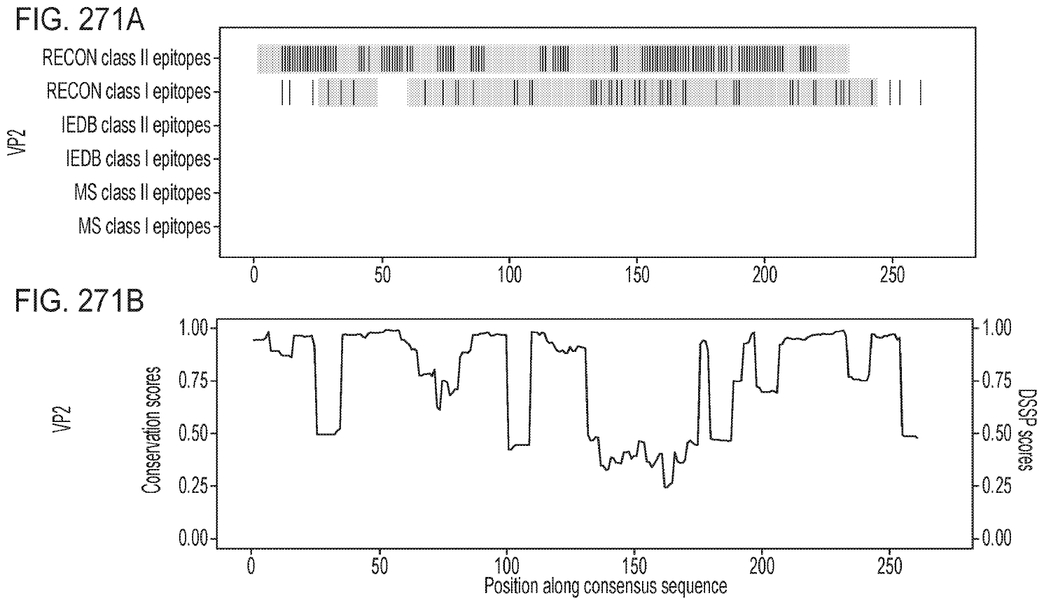

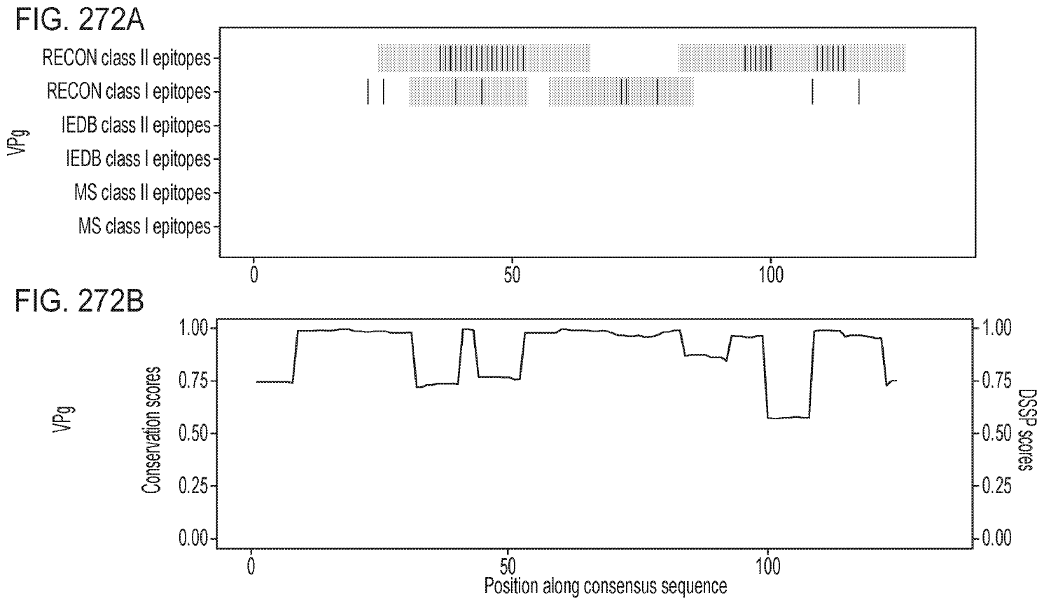

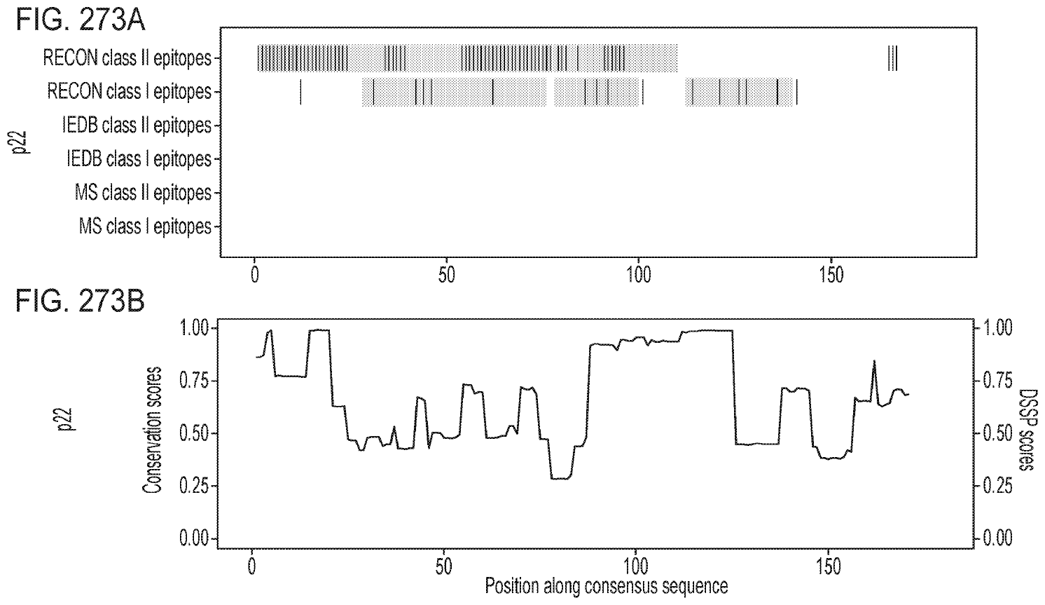

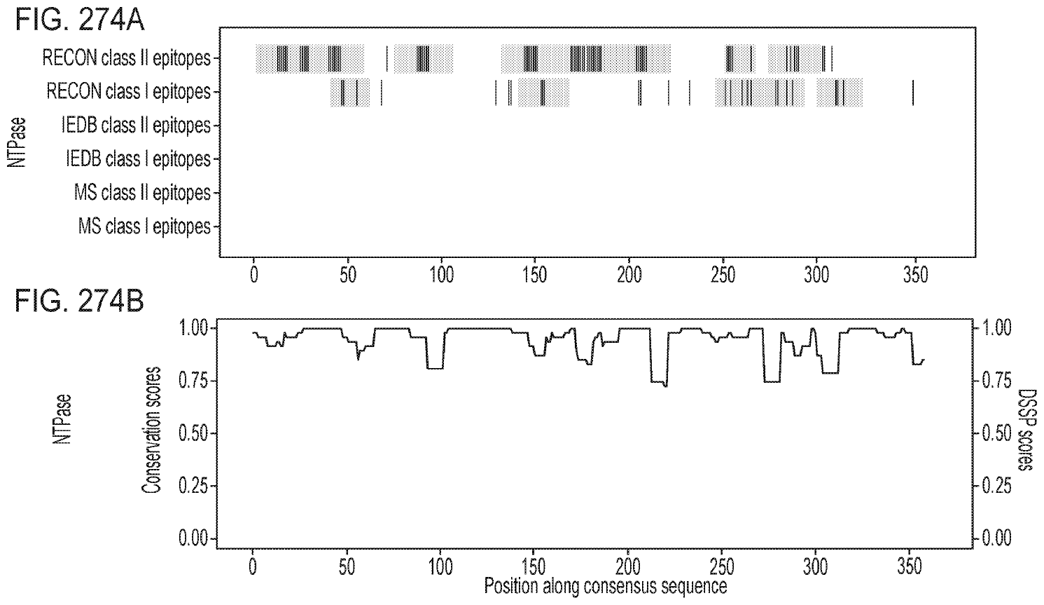

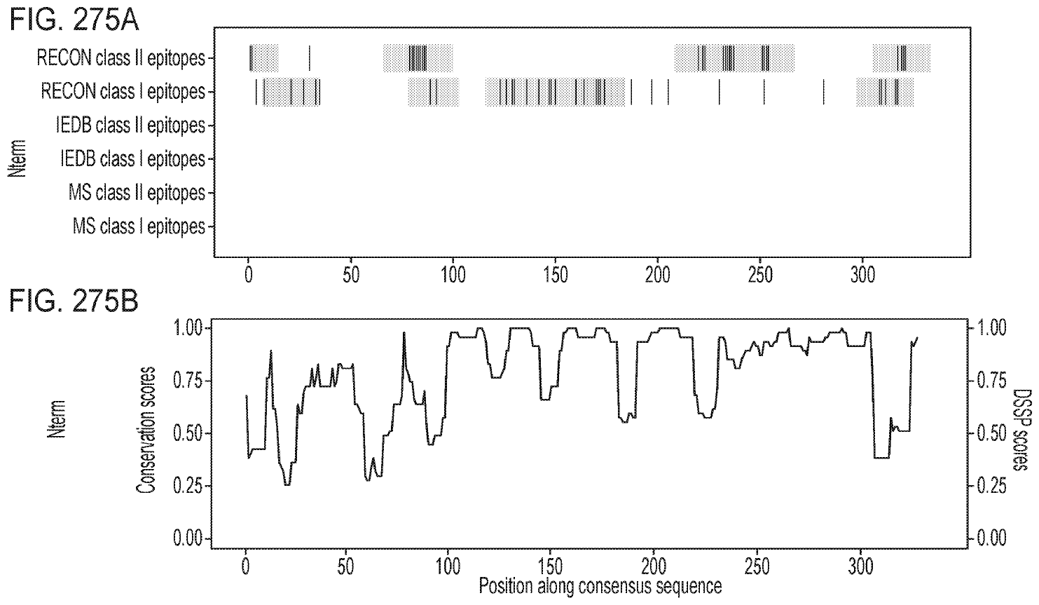

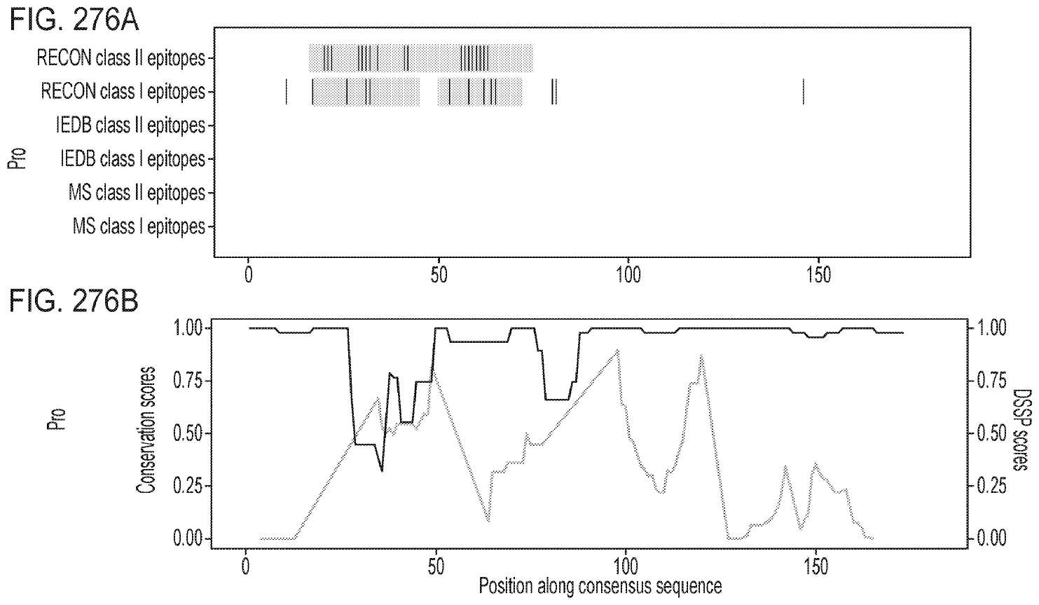

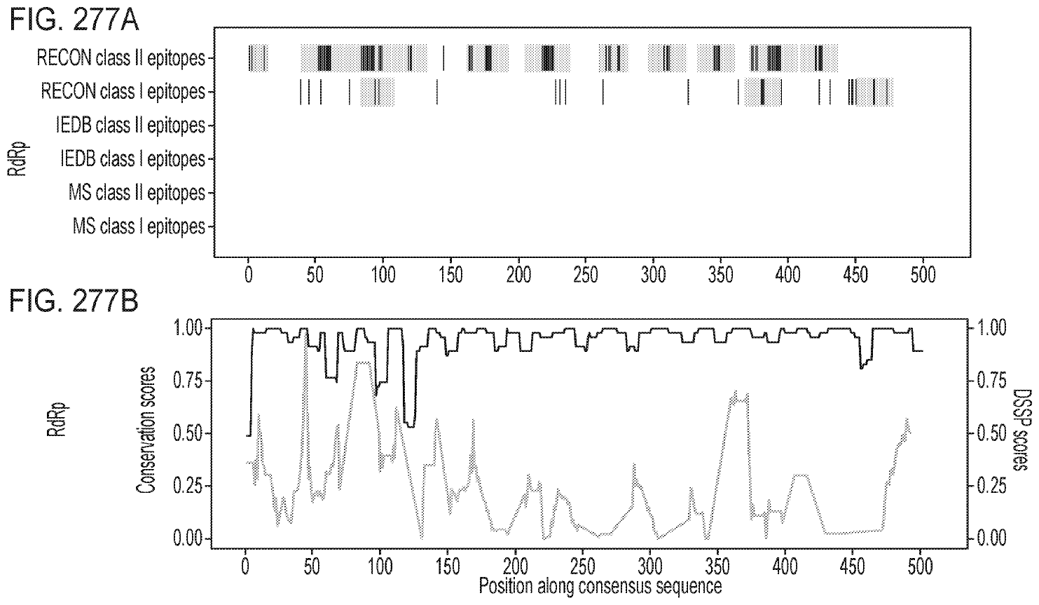

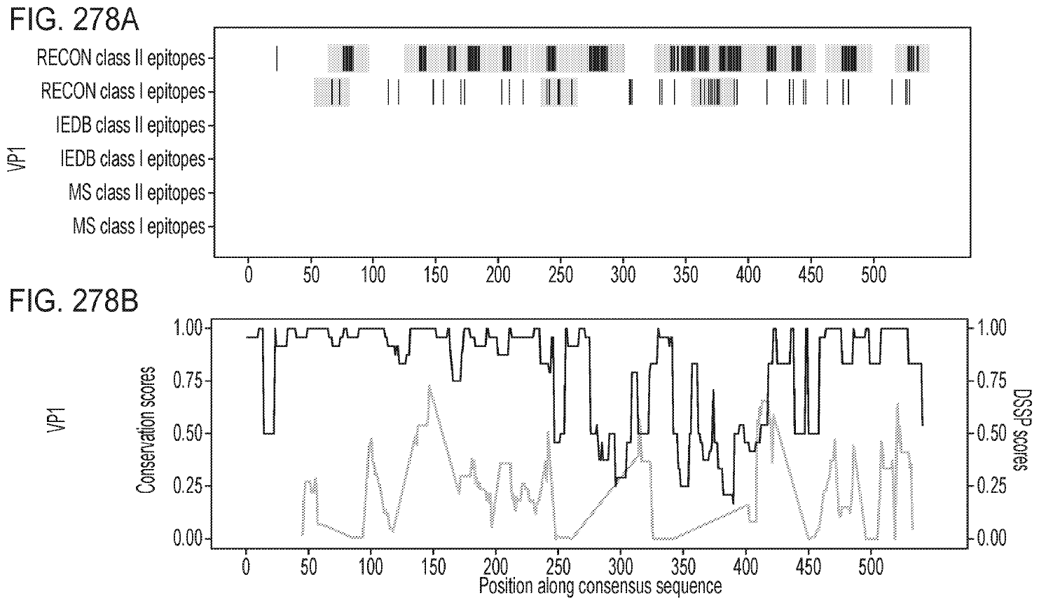

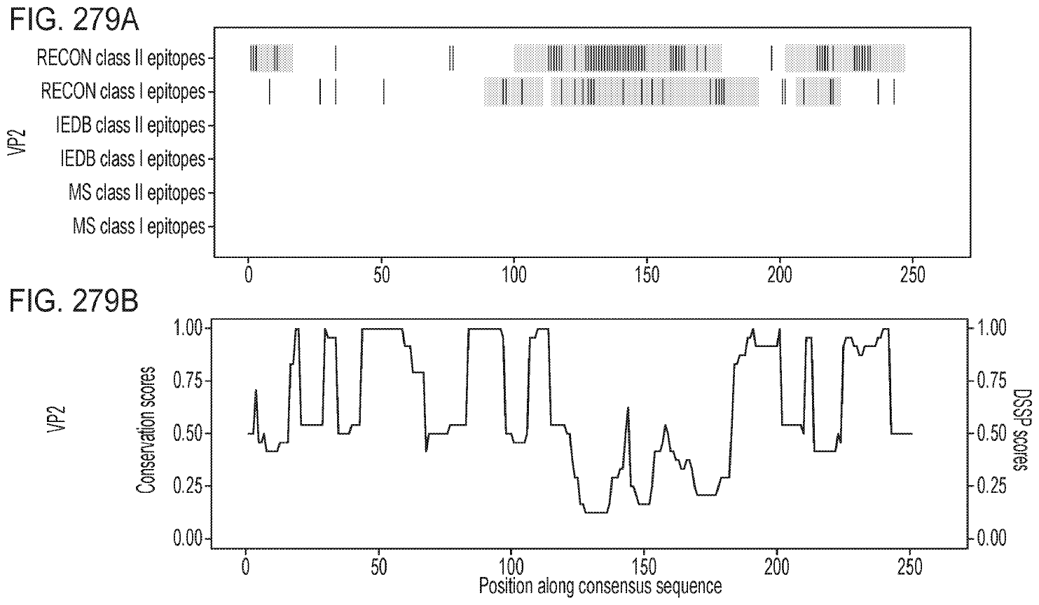

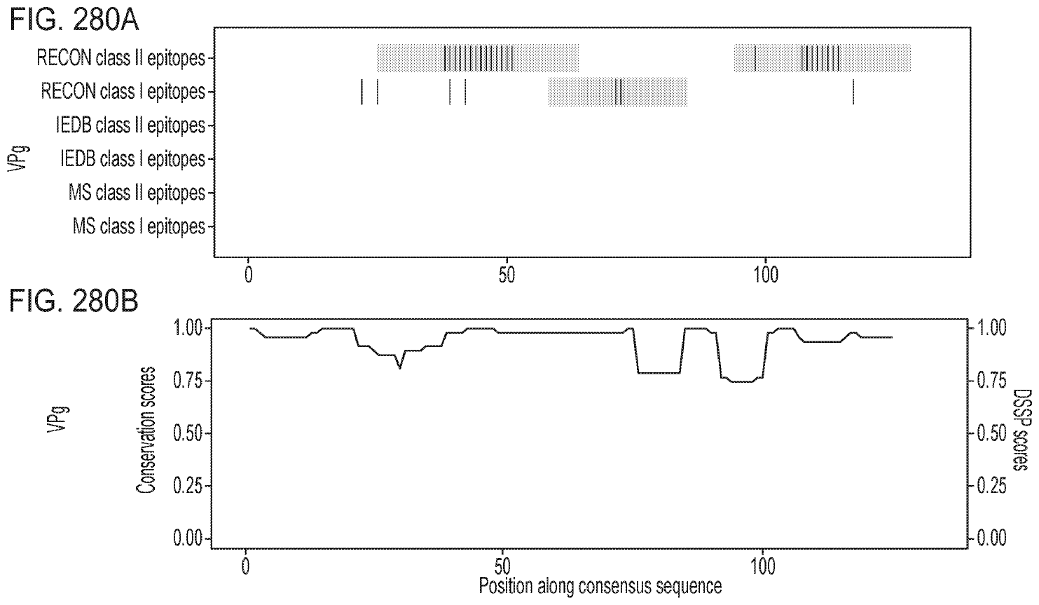

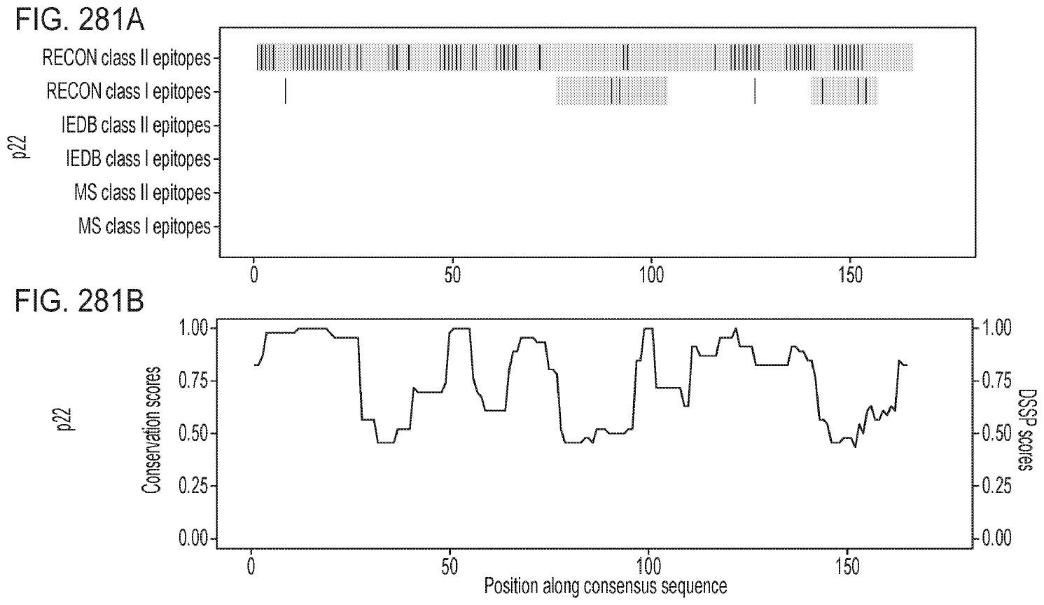

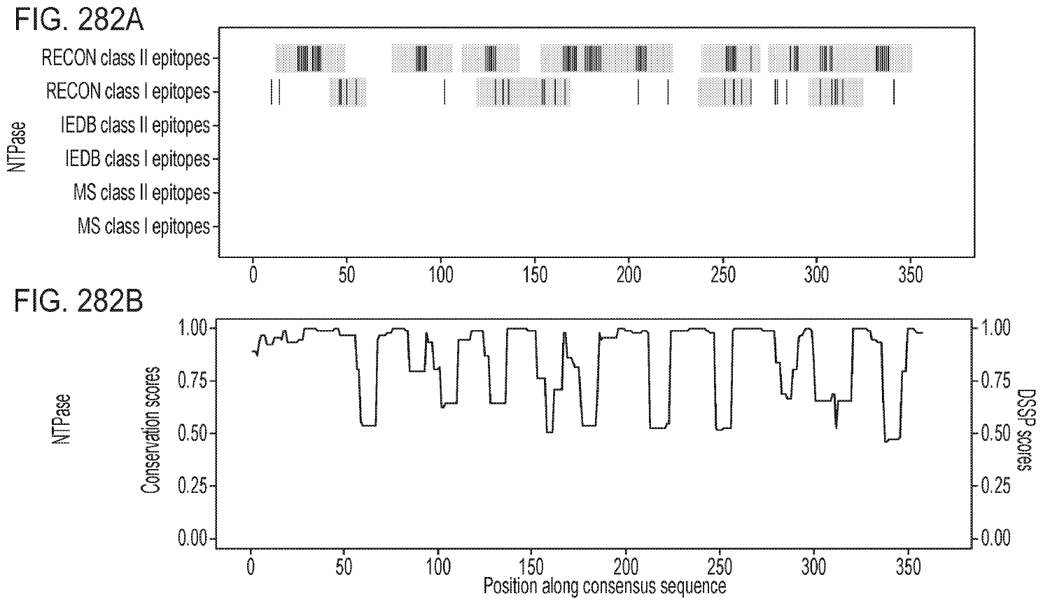

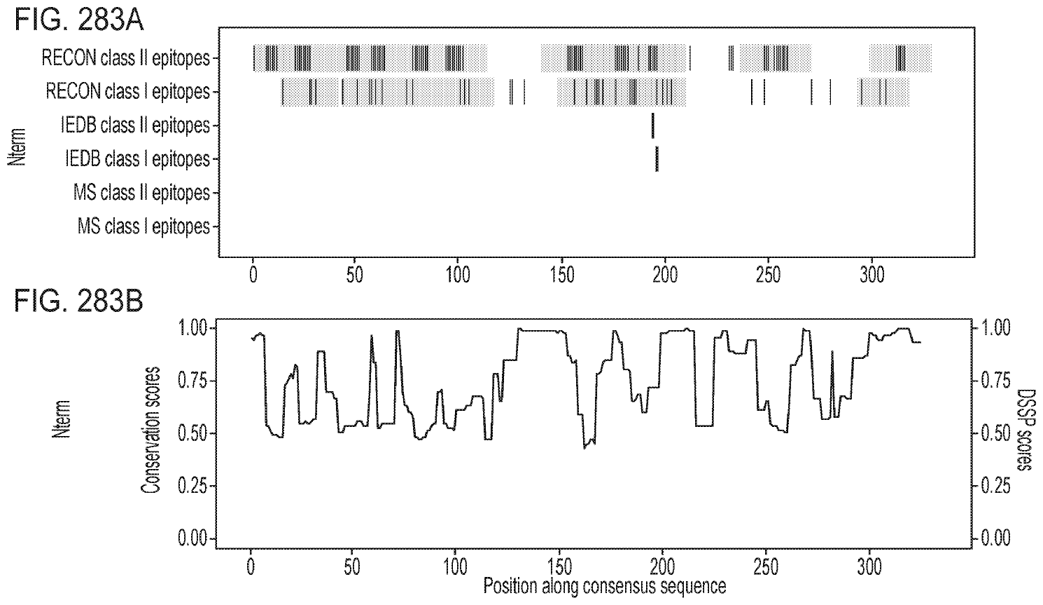

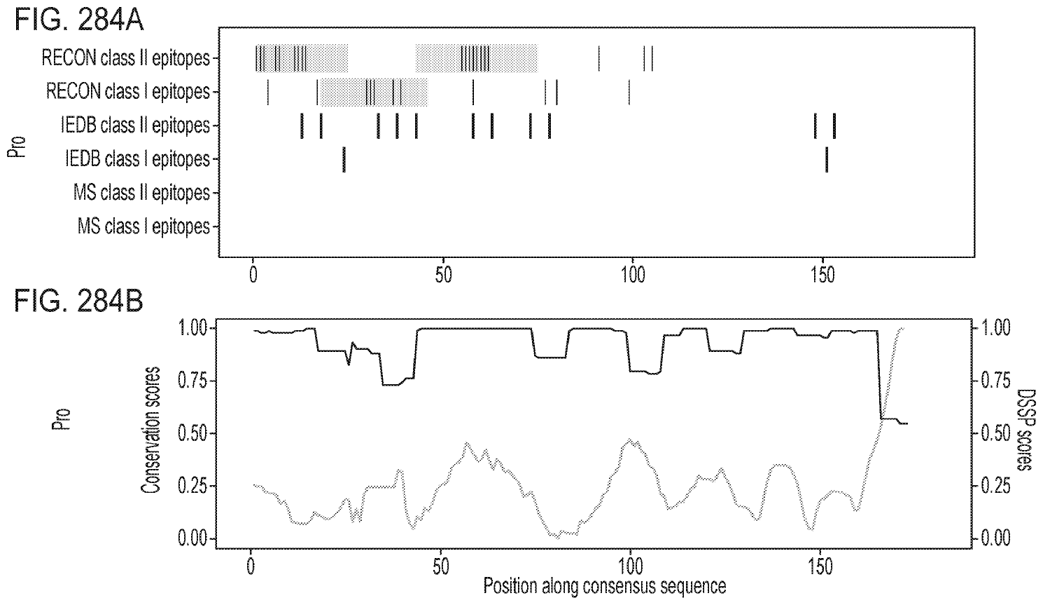

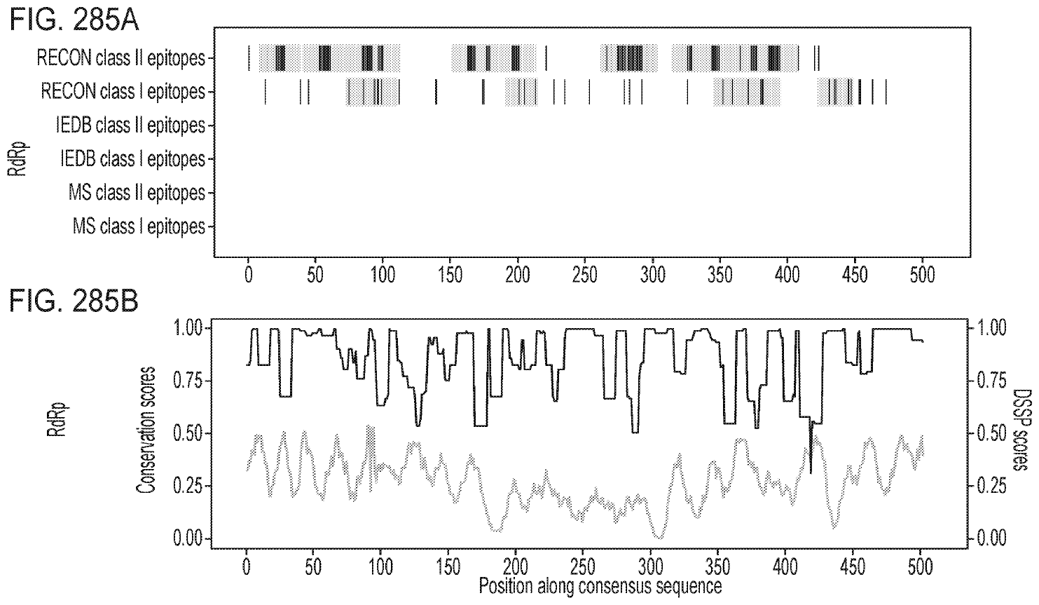

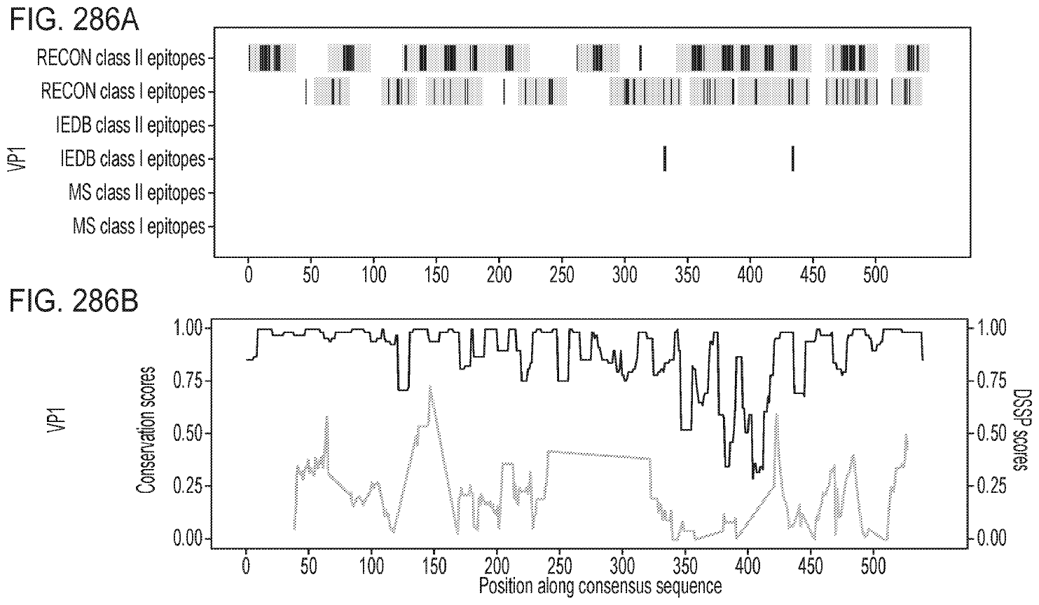

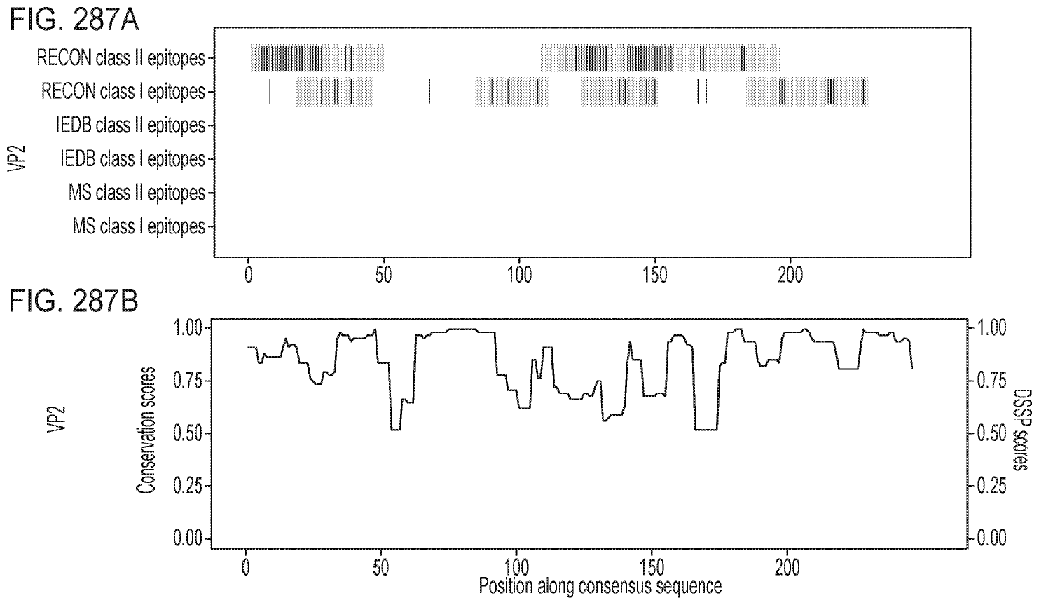

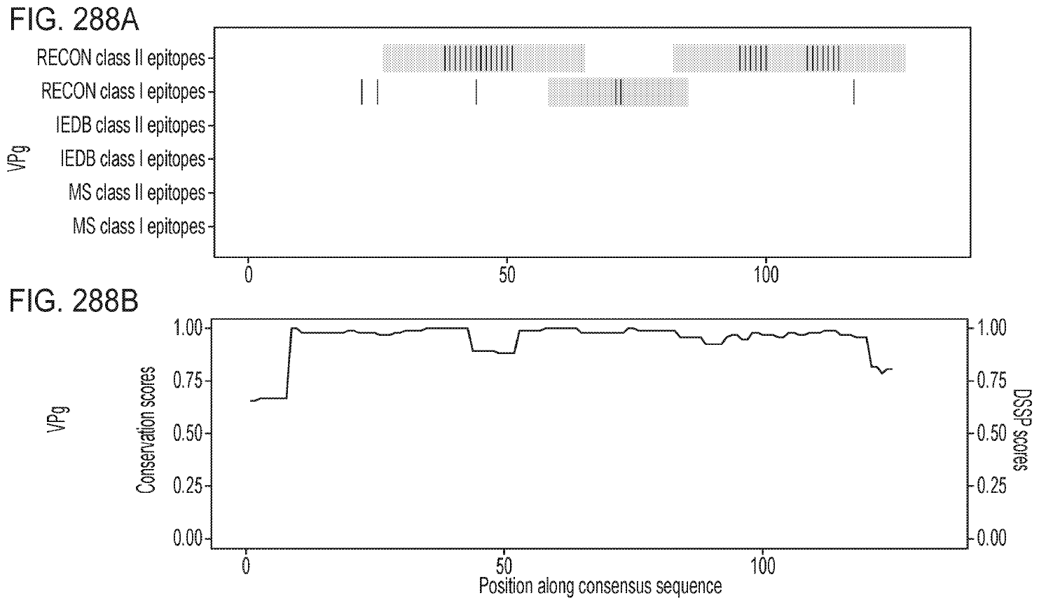

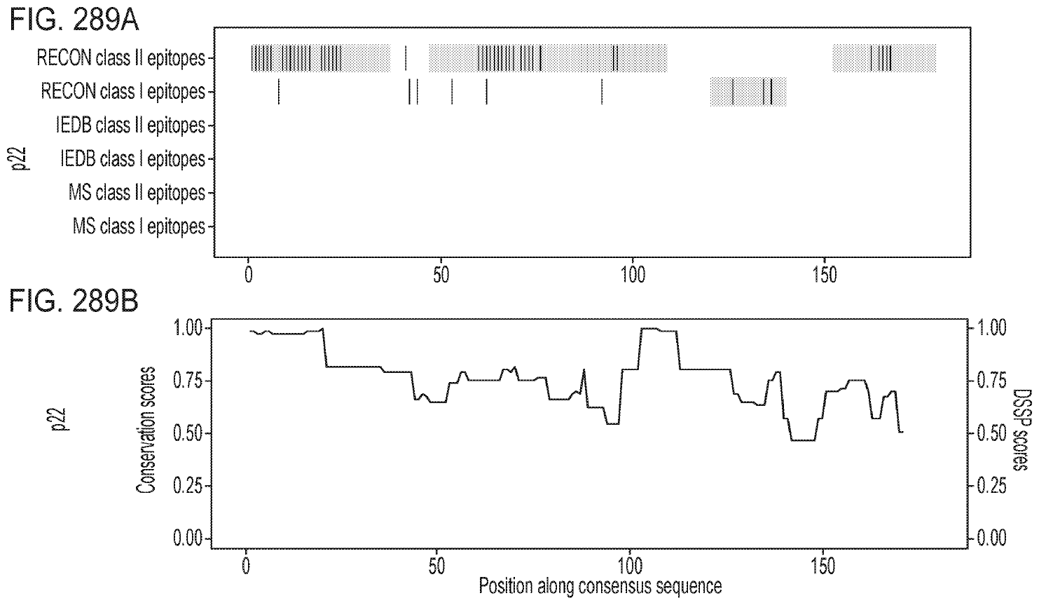

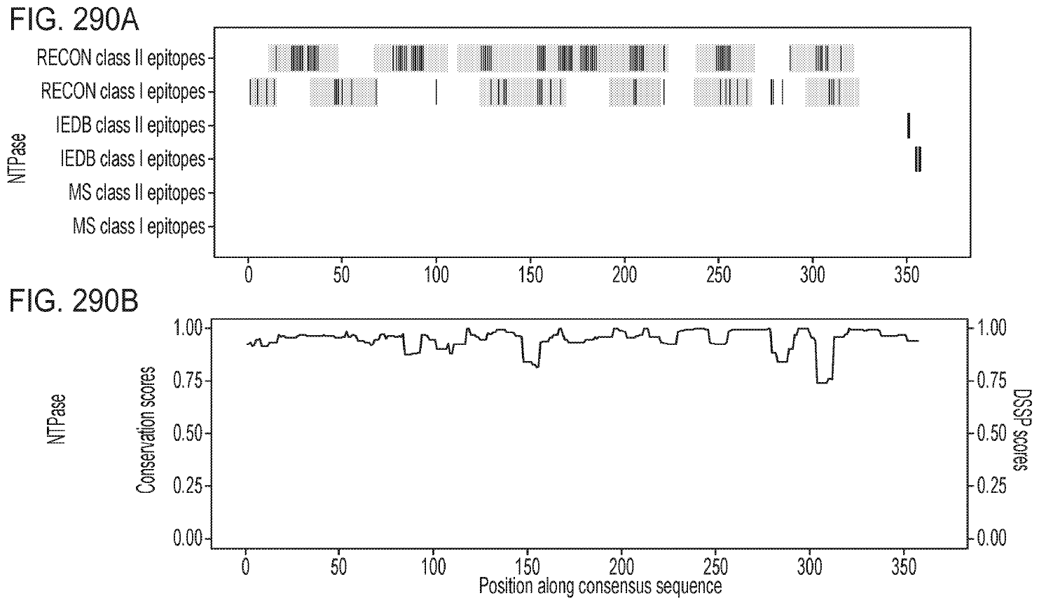

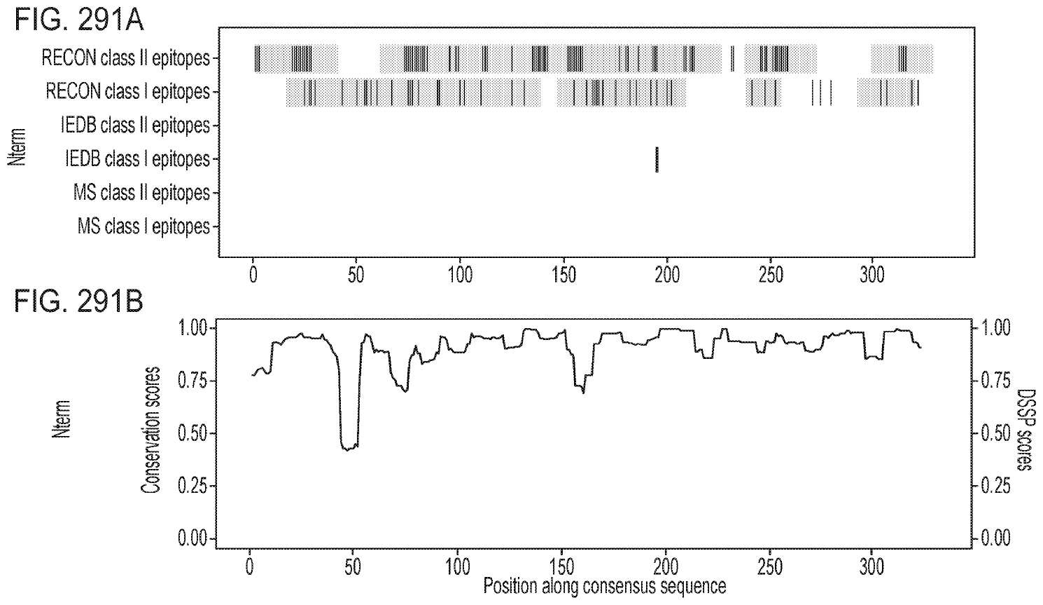

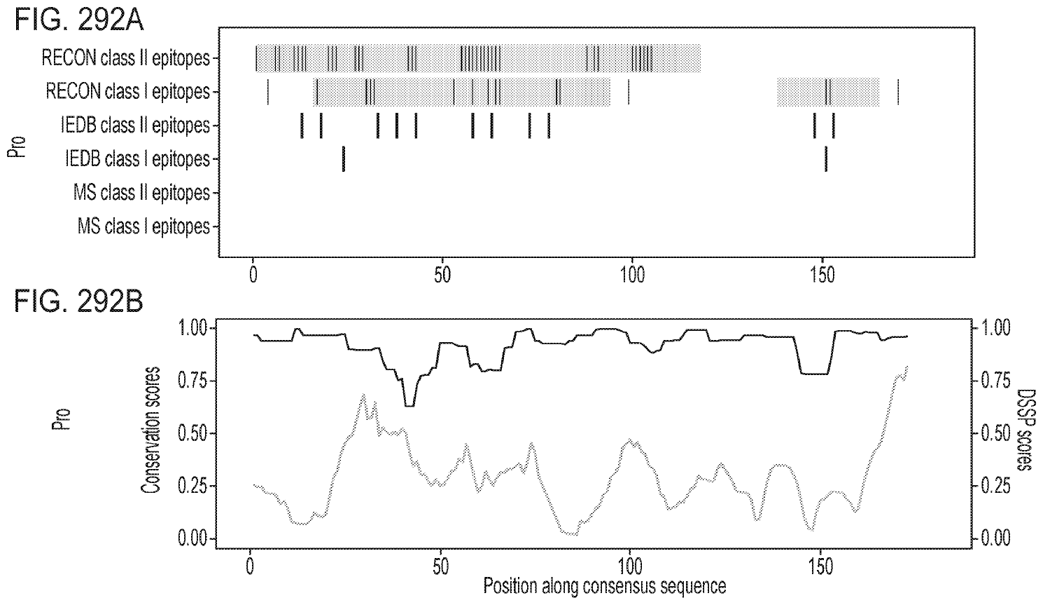

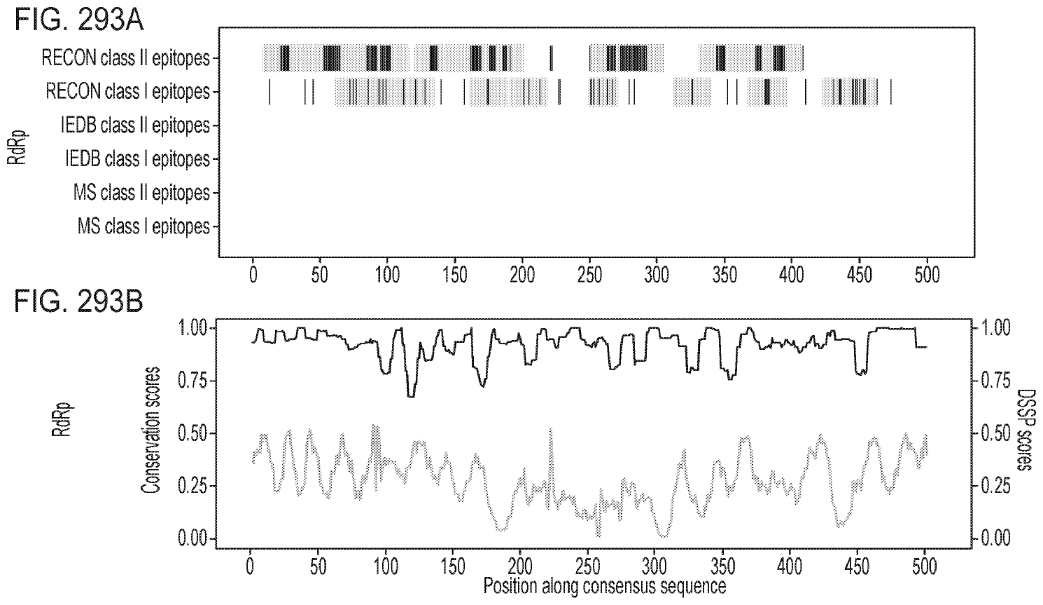

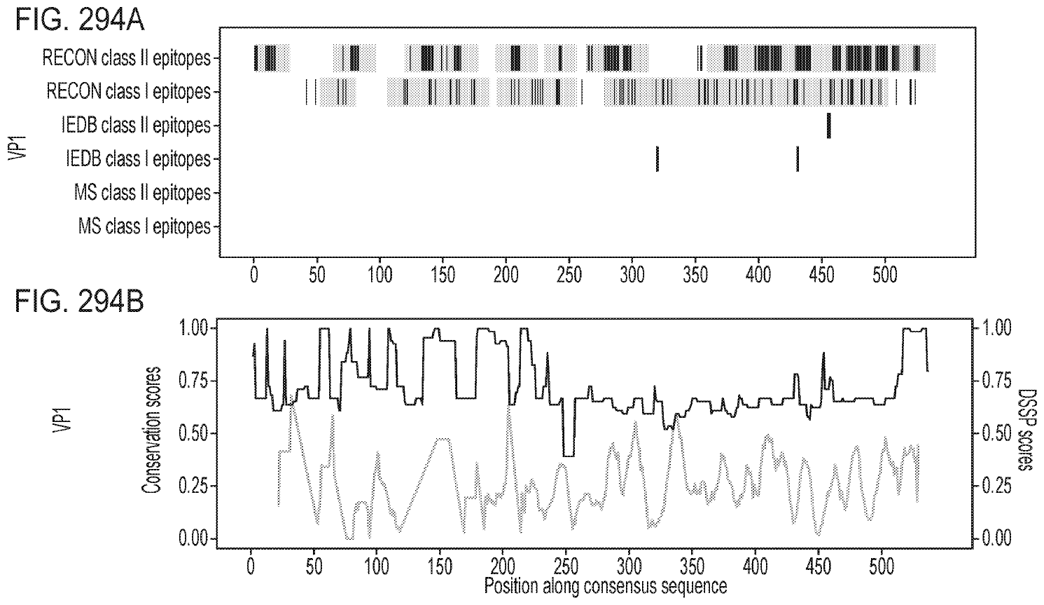

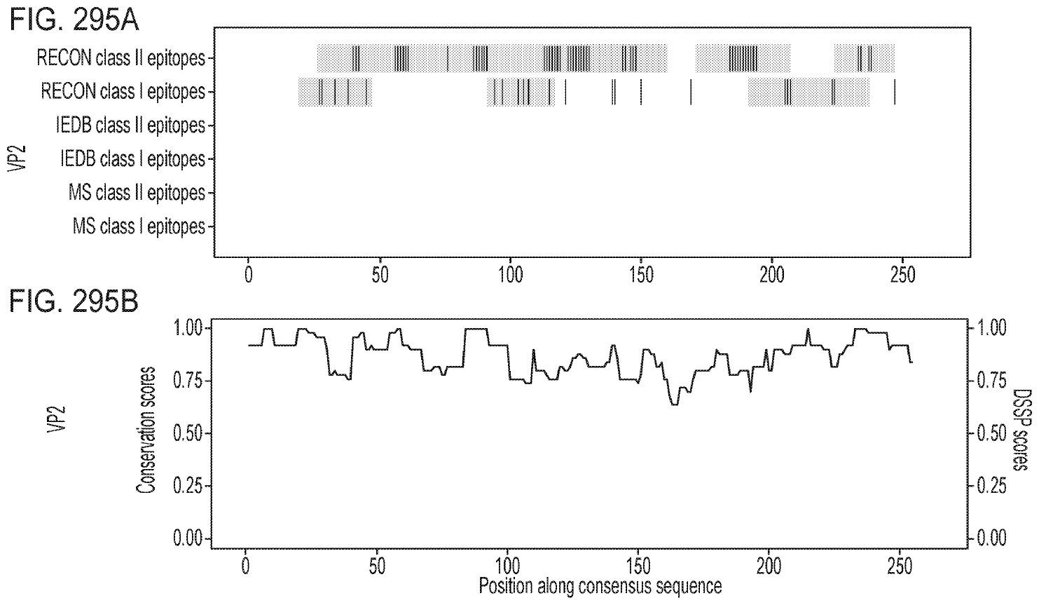

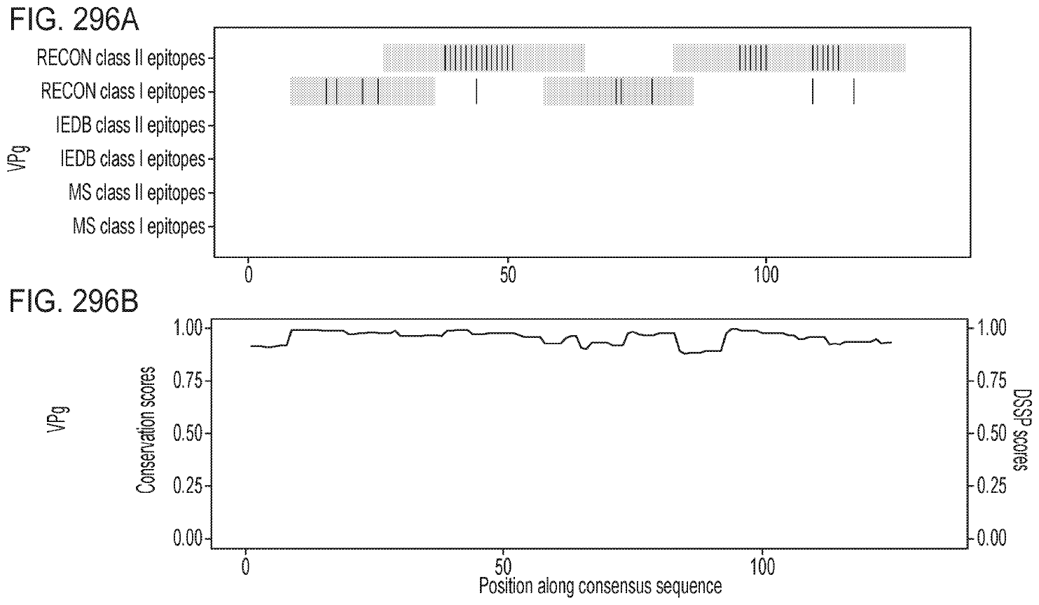

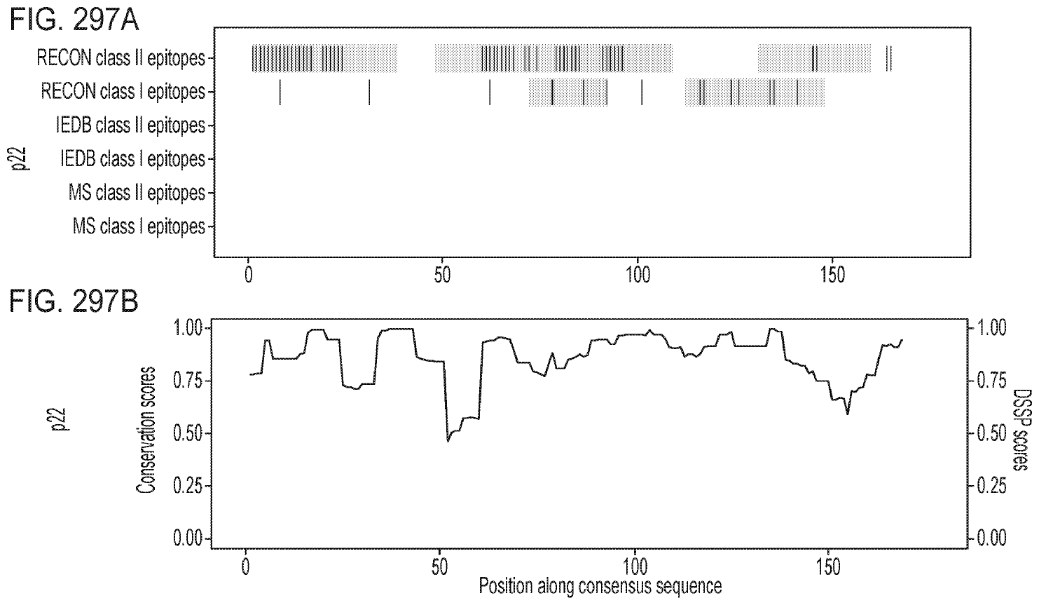

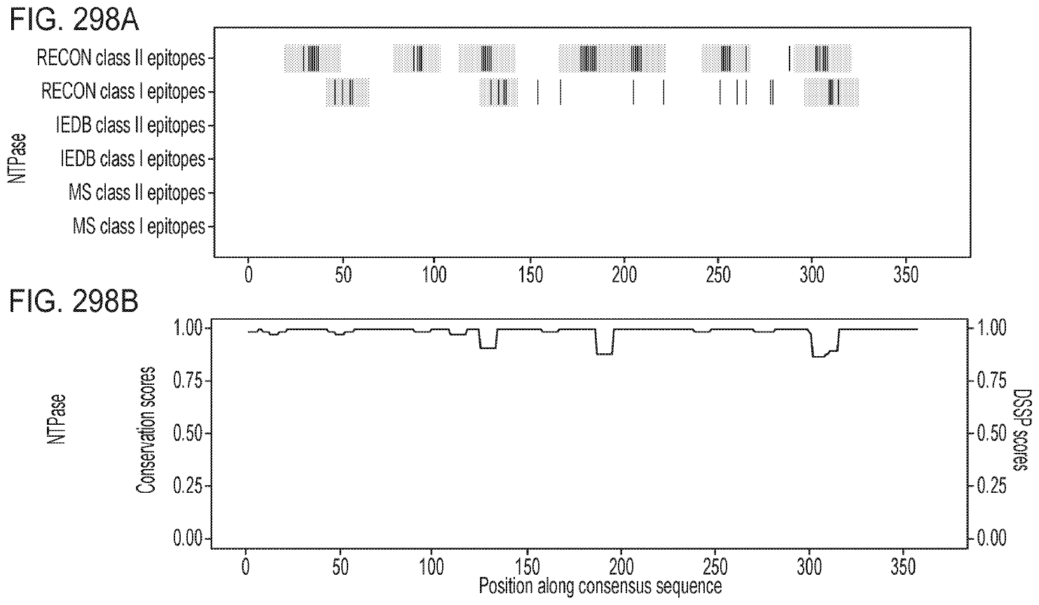

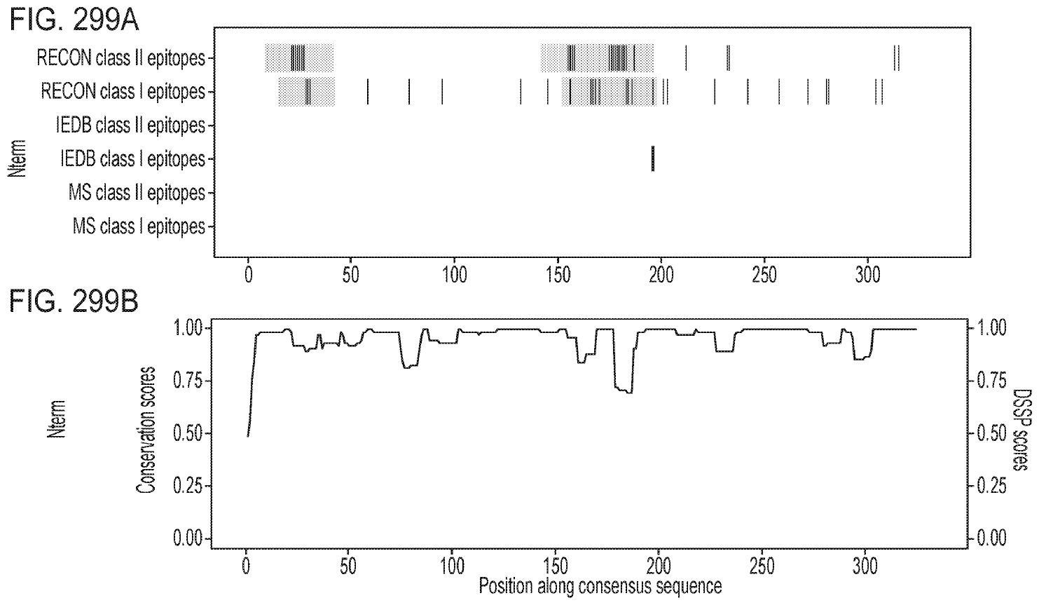

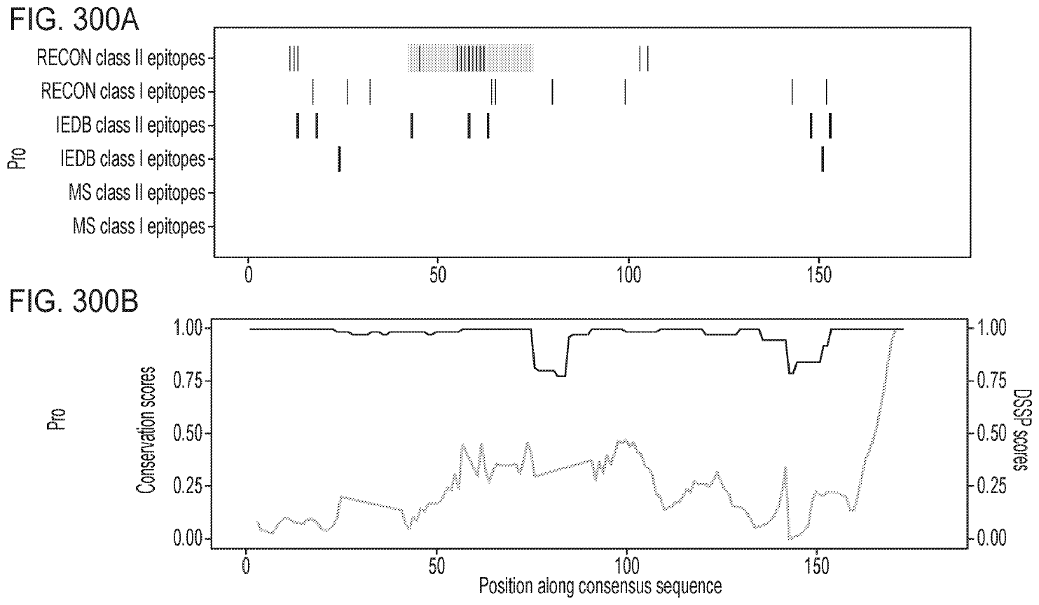

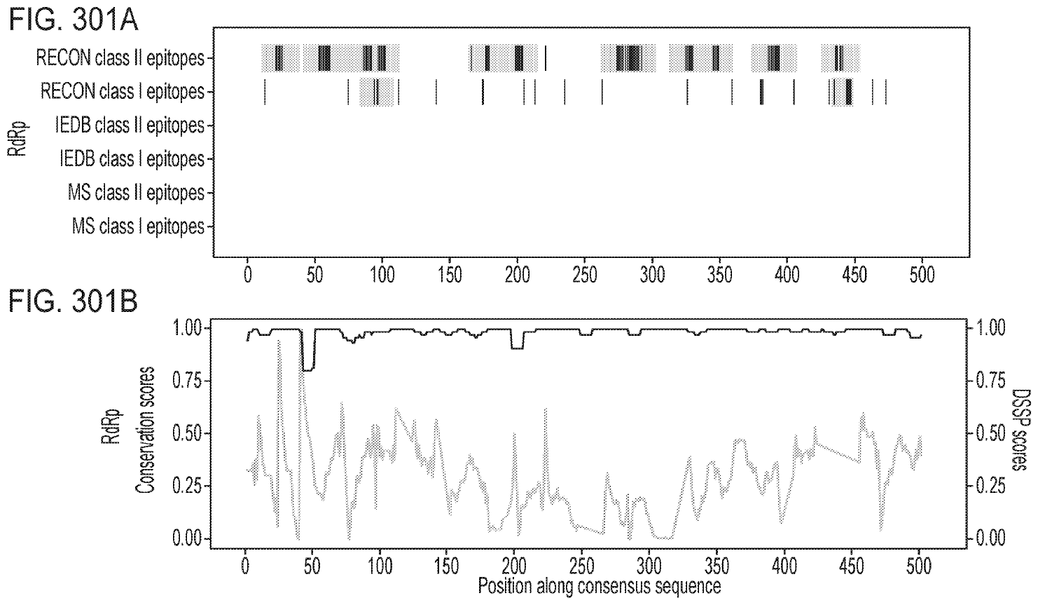

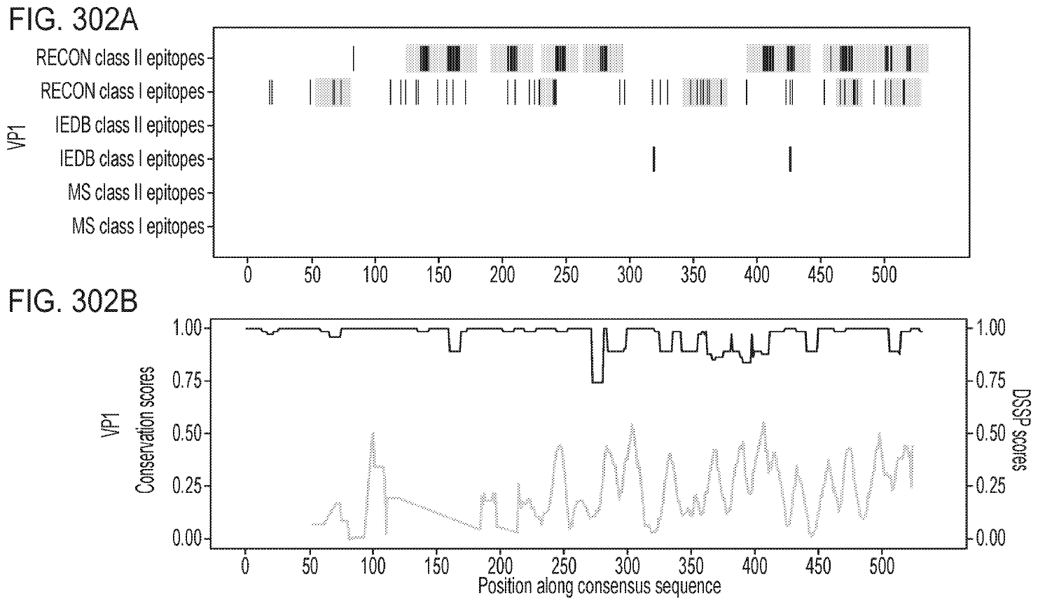

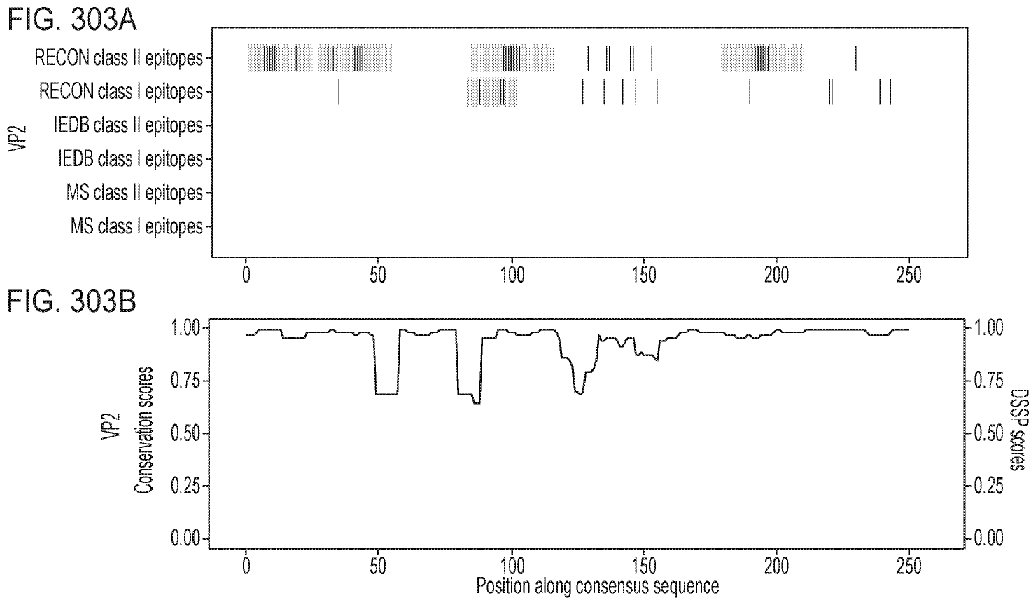

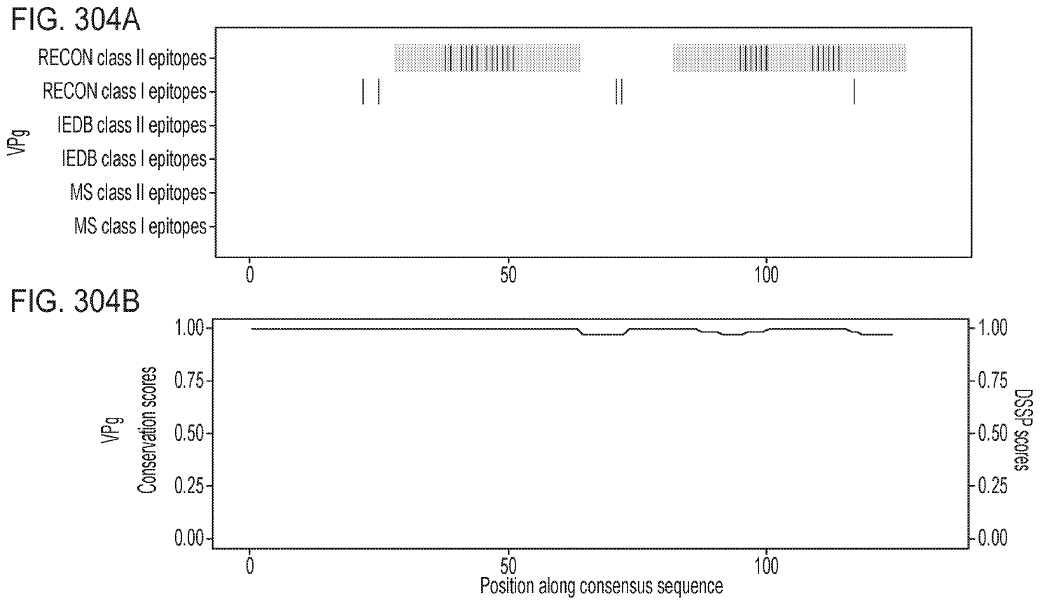

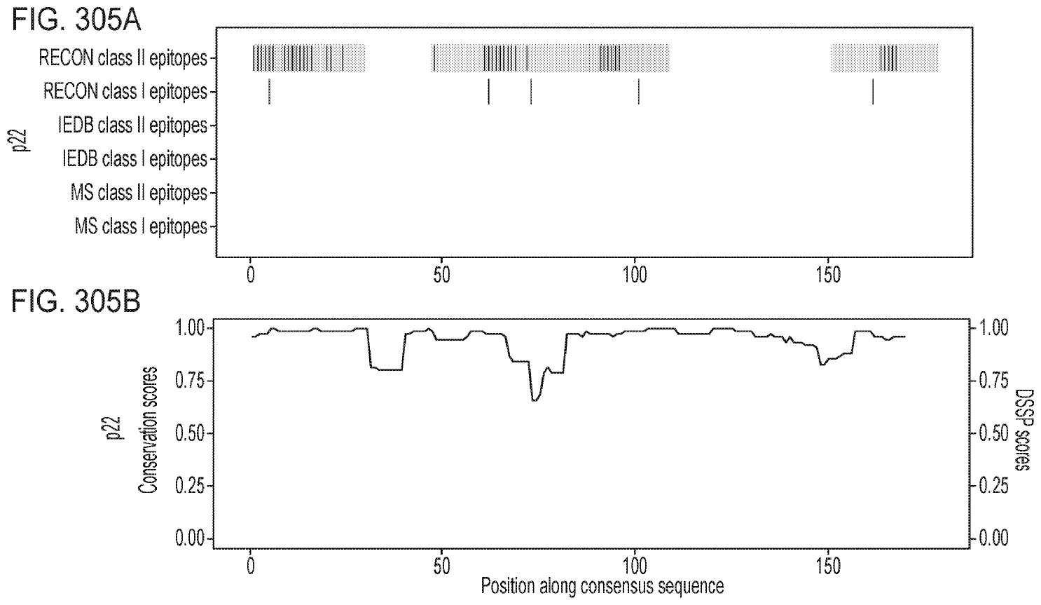

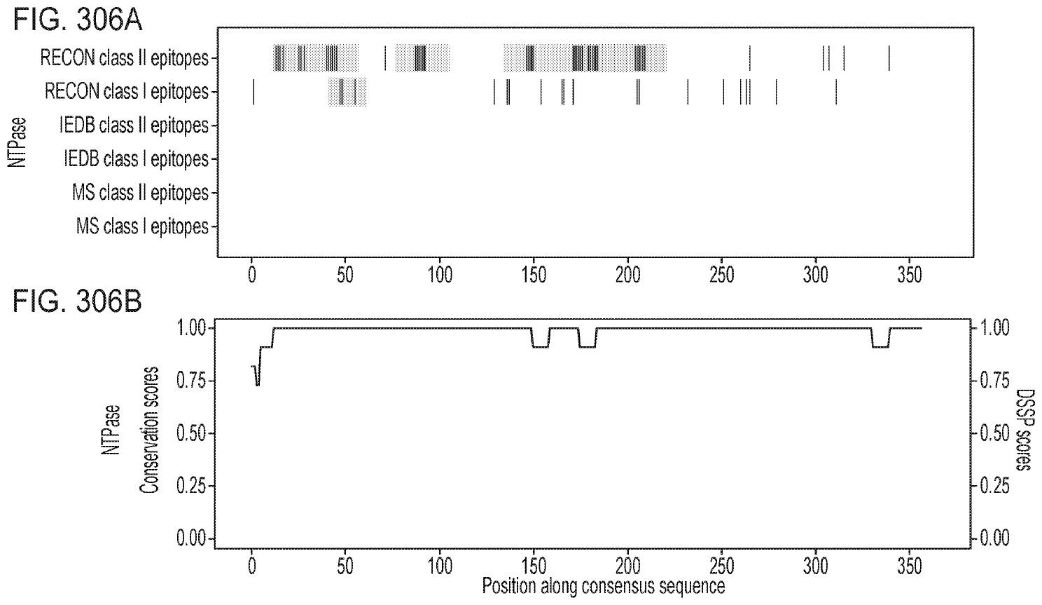

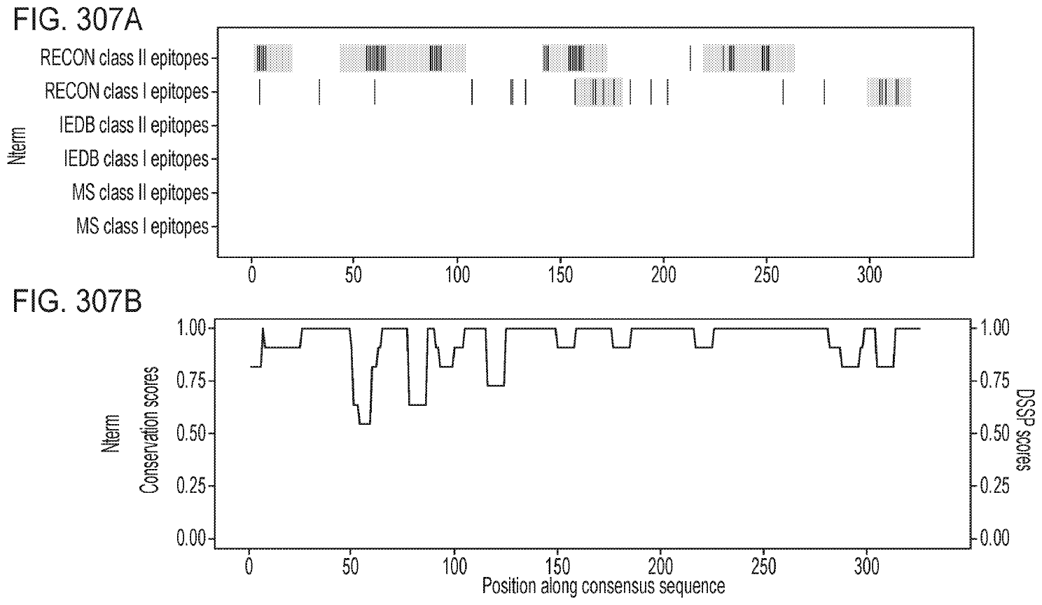

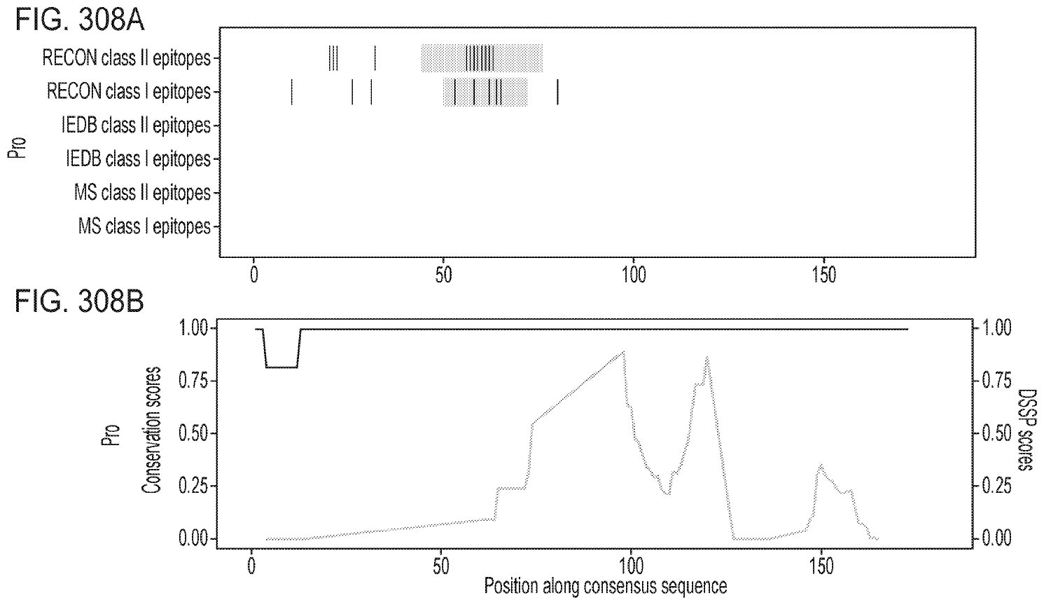

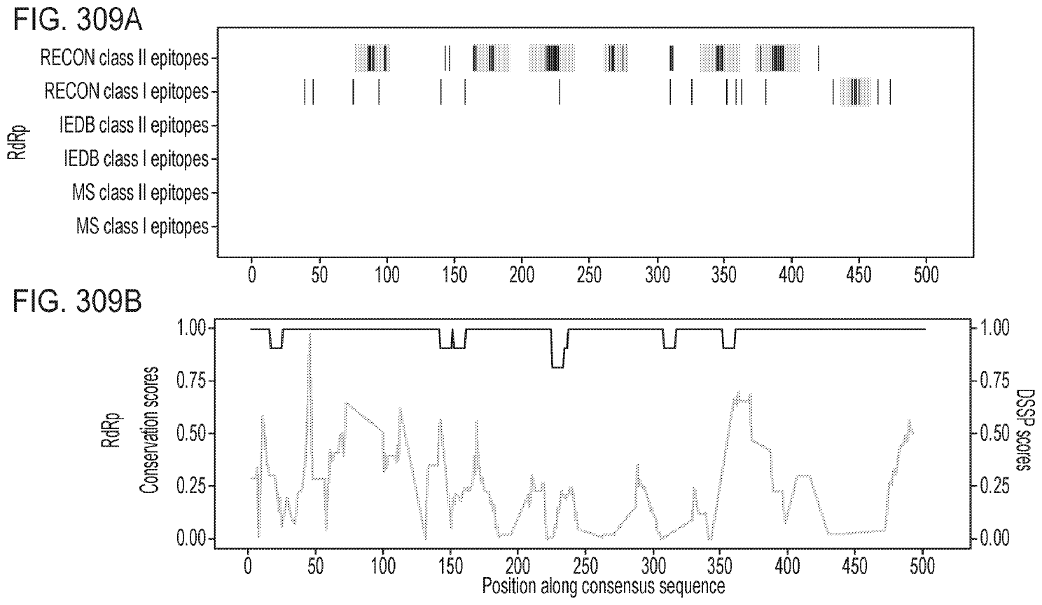

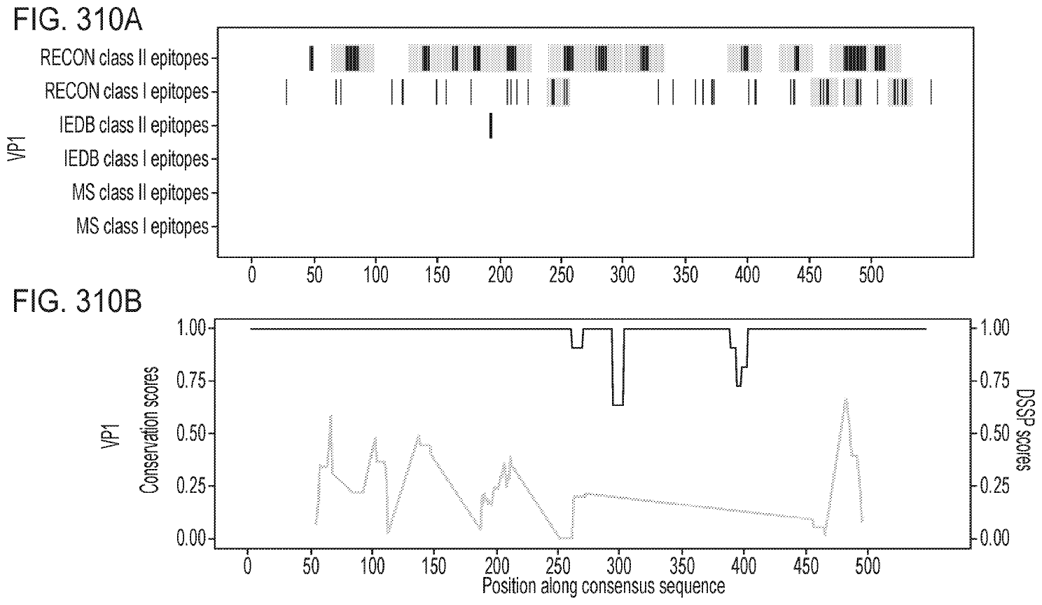

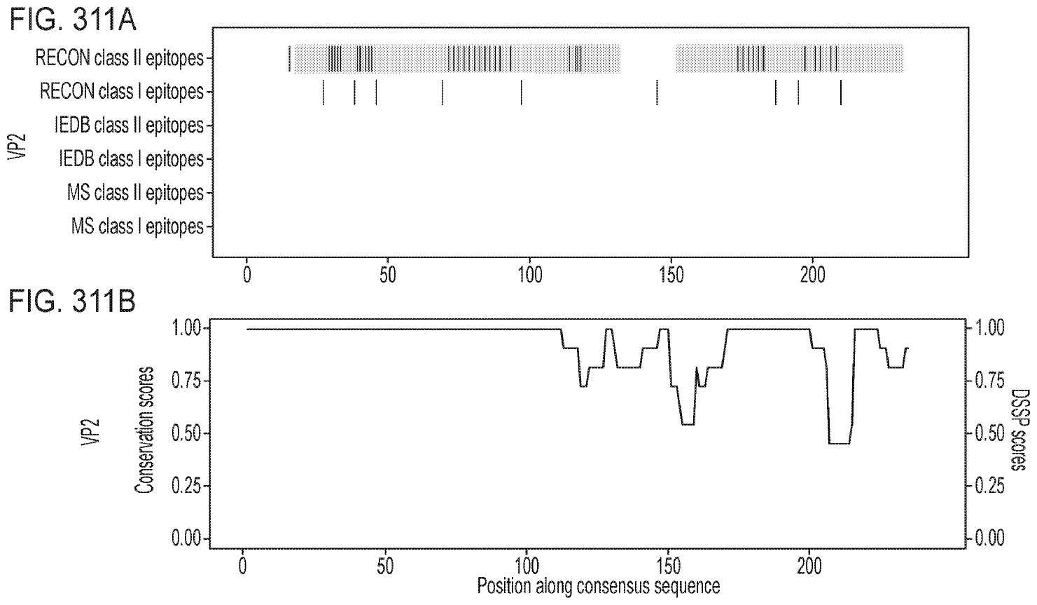

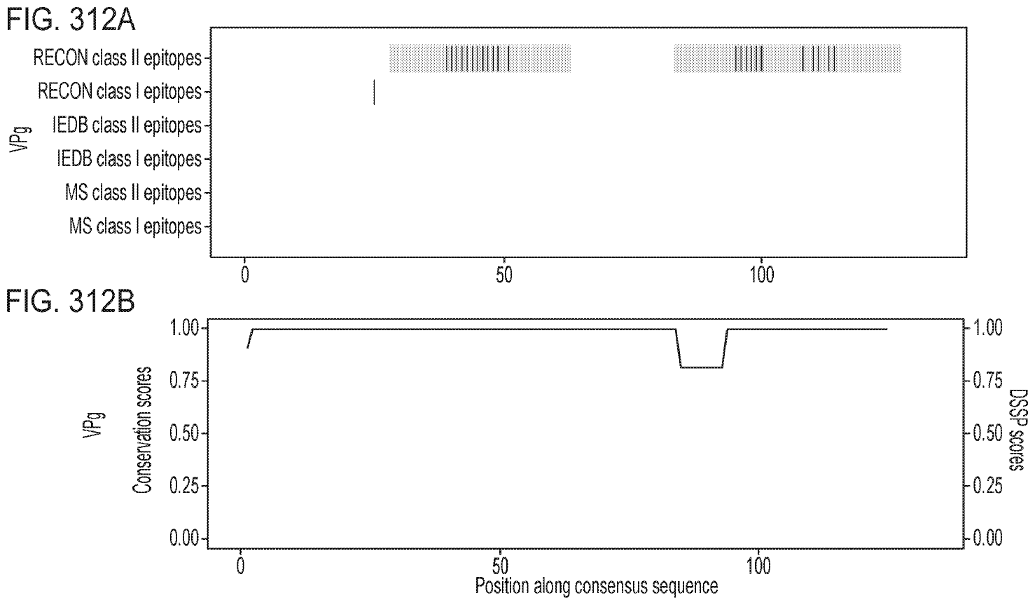

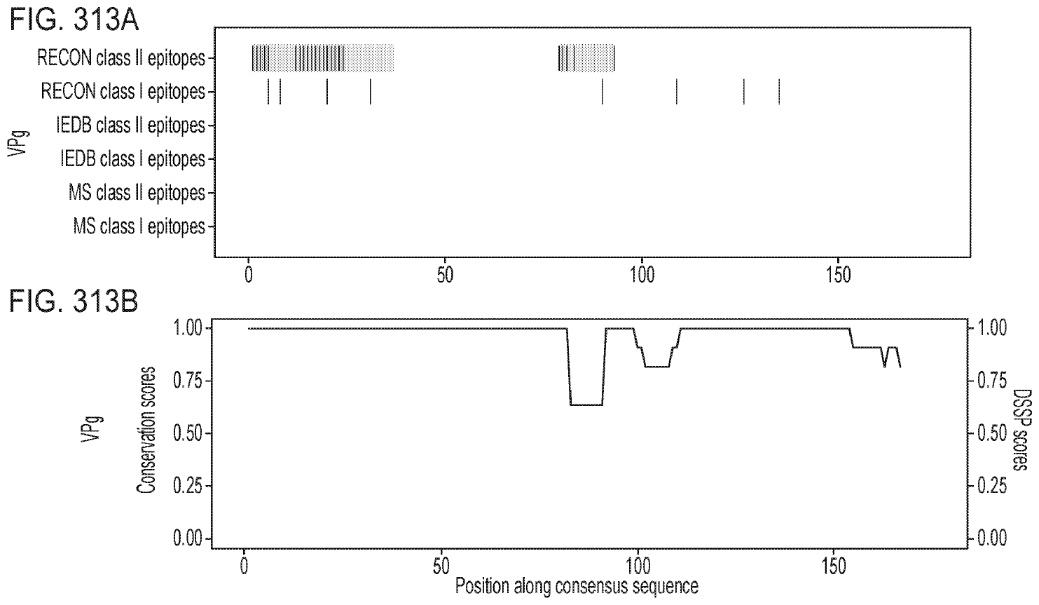

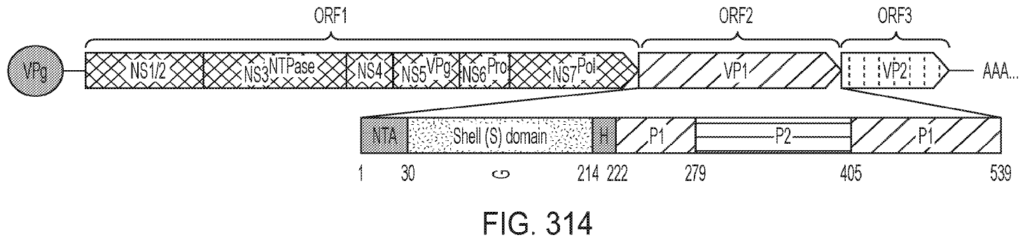

FIGS. 258A-313B show cross-strain sequence conservation and epitopes of interest for an indicated NoV gene. Conservation is determined according to a sliding 9mer window as described above, where 0 indicates no conservation and 1 indicates that all strains are identical in the given region. Solvent accessibility (per experimentally resolved structures) is also represented (likewise using a 9mer rolling average). Epitopes of interest are plotted with notch marks and include those highly predicted by RECON (percent rank <0.1 for HLA-I; percent rank <0.5 for HLA-II; for alleles common among individuals with European ancestry), identified by mass spectrometry-based HLA ligandomics, or otherwise identified in The Immune Epitope Database (iedb.org). It should be noted that positions are indexed with respect to the multiple sequence alignment, which contains gap characters. Areas enriched in HLA-I or HLA-II epitopes are highlighted with gray rectangles.

### Example 33: Exemplary Methods for Determination of NoV “Block” Regions and String Design

The present Example provides exemplary methods for identifying “block” regions of a NoV polypeptide that may be useful for immunogenic compositions (e.g., vaccine compositions, and epitope binding agents, etc.). In some embodiments, “block” regions of a NoV polypeptide can be expressed by NoV during a latent phase of its life cycle and/or during an active infection. In some embodiments, such “block” regions can be or comprise CD4+ and/or CD8+ T cell epitopes. In some embodiments, such “block” regions can be or comprise B cell epitopes. In some embodiments, such “block” regions can be or comprise predominantly CD4+ and/or CD8+ T cell epitopes.

In some embodiments, one or more “block” regions identified in the present Example can be encoded by a polynucleotide. In some embodiments, such a polynucleotide can be a polyribonucleotide (e.g., in some embodiments mRNA). In some embodiments, a plurality of (including, e.g., at least two, at least three, at least four, at least five, at least six, at least seven, at least eight, at least nine, at least ten, or more) “block regions” identified in the present Example can be linked by one or more linkers. In some embodiments, such a polynucleotide can comprise a secretory signal sequence (e.g., in some embodiments at the N-terminal of the polynucleotide). In some embodiments, such a polynucleotide can comprise an MHC trafficking signal sequence (e.g., a MHC class I trafficking signal sequence; MITD sequence), for example, in some embodiments at the C-terminal of the polynucleotide.

### Norovirus Strains GIX:

For each NoV protein, a sliding window of 15 amino acid positions are applied from start to end over the multiple sequence alignment (MSA) profile over all the sequences belonging to a particular strain. The number of peptides highly scored by RECON (percent rank <0.1 for HLA-I; percent rank <0.5 for HLA-II) for alleles that are common in people of European ancestry (A*02:01, A*01:01, A*03:01, A*24:02, A*11:01, B*07:02, B*08:01, B*44:02, B*35:01, B*44:03, C*07:01, C*07:02, C*04:01, C*06:02, C*03:04, DRB1*07:01, DRB1*15:01, DRB1*03:01, DRB1*01:01, DRB1*04:01, DRB4*01:03, DRB3*01:01, DRB3*02:02, DRB5*01:01, DRB3*03:01, DPA1*01:03/DPB1*04:01, DPA1*01:03/DPB1*02:01, DPA1*01:03/DPB1*04:02, DPA1*01:03/DPB1*03:01, DPA1*02:01/DPB1*01:01, DQA1*01:02/DQB1*06:02, DQA1*05:01/DQB1*02:01, DQA1*02:01/DQB1*02:02, DQA1*05:05/DQB1*03:01, DQA1*01:01/DQB1*05:01; in some embodiments, these allele selections could be revised to match the allele frequencies applicable in the region(s) of vaccine distribution) that map to each of these windows are counted. Sliding windows with at least 5 such peptides are considered as “dense epitope regions”. Dense epitope regions are determined for HLA-I and HLA-II peptides separately, and for each type of peptides, dense epitope regions are merged into continuous blocks. These continuous blocks are then filtered to only contain intervals where the corresponding protein conservation scores previously determined are higher than 0.9.

### Norovirus Strains GII.P4:

For each NoV protein, a sliding window of 15 amino acid positions are applied from start to end over the multiple sequence alignment (MSA) profile over all the sequences belonging to a particular strain. The number of peptides highly scored by RECON (percent rank <0.1 for HLA-I; percent rank <0.5 for HLA-II) for alleles that are common in people of European ancestry (A*02:01, A*01:01, A*03:01, A*24:02, A*11:01, B*07:02, B*08:01, B*44:02, B*35:01, B*44:03, C*07:01, C*07:02, C*04:01, C*06:02, C*03:04, DRB1*07:01, DRB1*15:01, DRB1*03:01, DRB1*01:01, DRB1*04:01, DRB4*01:03, DRB3*01:01, DRB3*02:02, DRB5*01:01, DRB3*03:01, DPA1*01:03/DPB1*04:01, DPA1*01:03/DPB1*02:01, DPA1*01:03/DPB1*04:02, DPA1*01:03/DPB1*03:01, DPA1*02:01/DPB1*01:01, DQA1*01:02/DQB1*06:02, DQA1*05:01/DQB1*02:01, DQA1*02:01/DQB1*02:02, DQA1*05:05/DQB1*03:01, DQA1*01:01/DQB1*05:01; in some embodiments, these allele selections could be revised to match the allele frequencies applicable in the region(s) of vaccine distribution) that map to each of these windows are counted. Sliding windows with at least 15 such peptides are considered as “dense epitope regions.” Dense epitope regions are determined for HLA-I and HLA-II peptides separately, and for each type of peptides, dense epitope regions are merged into continuous blocks. These continuous blocks are then filtered to only contain intervals where the corresponding protein conservation scores previously determined are higher than 0.9.

### Norovirus Strains GI, GII.P12, GII.P16 and GII.P17:

For each NoV protein, a sliding window of 15 amino acid positions are applied from start to end over the multiple sequence alignment (MSA) profile over all the sequences belonging to a particular strain. The number of peptides highly scored by RECON (percent rank <0.1 for HLA-I; percent rank <0.5 for HLA-II) for alleles that are common in people of European ancestry (A*02:01, A*01:01, A*03:01, A*24:02, A*11:01, B*07:02, B*08:01, B*44:02, B*35:01, B*44:03, C*07:01, C*07:02, C*04:01, C*06:02, C*03:04, DRB1*07:01, DRB1*15:01, DRB1*03:01, DRB1*01:01, DRB1*04:01, DRB4*01:03, DRB3*01:01, DRB3*02:02, DRB5*01:01, DRB3*03:01, DPA1*01:03/DPB1*04:01, DPA1*01:03/DPB1*02:01, DPA1*01:03/DPB1*04:02, DPA1*01:03/DPB1*03:01, DPA1*02:01/DPB1*01:01, DQA1*01:02/DQB1*06:02, DQA1*05:01/DQB1*02:01, DQA1*02:01/DQB1*02:02, DQA1*05:05/DQB1*03:01, DQA1*01:01/DQB1*05:01; in some embodiments, these allele selections could be revised to match the allele frequencies applicable in the region(s) of vaccine distribution) that map to each of these windows are counted. Sliding windows with at least 10 such peptides are considered as “dense epitope regions.” Dense epitope regions are determined for HLA-I and HLA-II peptides separately, and for each type of peptides, dense epitope regions are merged into continuous blocks. These continuous blocks are then filtered to only contain intervals where the corresponding protein conservation scores previously determined are higher than 0.9.

### Norovirus Strain GII.P7:

For each NoV protein, a sliding window of 15 amino acid positions are applied from start to end over the multiple sequence alignment (MSA) profile over all the sequences belonging to a particular strain. The number of peptides highly scored by RECON (percent rank <0.1 for HLA-I; percent rank <0.5 for HLA-II) for alleles that are common in people of European ancestry (A*02:01, A*01:01, A*03:01, A*24:02, A*11:01, B*07:02, B*08:01, B*44:02, B*35:01, B*44:03, C*07:01, C*07:02, C*04:01, C*06:02, C*03:04, DRB1*07:01, DRB1*15:01, DRB1*03:01, DRB1*01:01, DRB1*04:01, DRB4*01:03, DRB3*01:01, DRB3*02:02, DRB5*01:01, DRB3*03:01, DPA1*01:03/DPB1*04:01, DPA1*01:03/DPB1*02:01, DPA1*01:03/DPB1*04:02, DPA1*01:03/DPB1*03:01, DPA1*02:01/DPB1*01:01, DQA1*01:02/DQB1*06:02, DQA1*05:01/DQB1*02:01, DQA1*02:01/DQB1*02:02, DQA1*05:05/DQB1*03:01, DQA1*01:01/DQB1*05:01; in some embodiments, these allele selections could be revised to match the allele frequencies applicable in the region(s) of vaccine distribution) that map to each of these windows are counted. Sliding windows with at least 5 (HLA-I) or 10 (HLA-II) such peptides are considered as “dense epitope regions.” Dense epitope regions are determined for HLA-I and HLA-II peptides separately, and for each type of peptides, dense epitope regions are merged into continuous blocks. These continuous blocks are then filtered to only contain intervals where the corresponding protein conservation scores previously determined are higher than 0.9.

For each block identified from the above steps, the reference sequence for the corresponding virus is then used to determine sequence—sequences encompassing the block interval plus at least 10 amino acids (including, e.g., at least 11 amino acids, at least 12 amino acids, at least 13 amino acids, at least 14 amino acids, at least 15 amino acids, or more) downstream and upstream of the block interval are taken to form chunk sequences. In some embodiments, sequences encompassing the block interval plus 14 amino acids downstream and 15 amino acids upstream of the block interval are taken to form chunk sequences. The chunk sequences are filtered to contain only prioritized genes, and linked together using linkers (e.g., but not limited to linkers described herein such as, e.g., GSS linkers) with a secretary signal peptide domain (SP domain) in the N terminal and MITD domain in the C terminal. Exemplary sequences for the linker, N-terminal domain, and C-terminal domain are provided as follows:
SP domain: MKMRVMAPRTLILLLSGALALTETWAGS
MITD domain: IVGIVAGLAVLAVVVIGAVVATVMCRRKSSGGKGGSYSQAASSDSAQGSDVSLTA
Start GSS linker (after SP domain): GGSGGGGSGG
Middle GSS linker (between blocks): GGSGGGGSGG
End GSS linker (before MITD): GGSLGGGGSG

The corresponding nucleotide sequence can be determined by reverse translation using the wildtype viral sequence, codon optimization, and/or some other codon selection scheme that optimizes one or more aspects of the resulting RNA string (e.g. stability, the lack of hairpins, etc.).

Tables 14A-14N contain CD8 and CD4-focused blocks, respectively, for each clade (including GI, GII.P4, GII.P7, GII.P12, GII.P16, GII.P17, and GIX) identified from the analyses as described above, as well as the corresponding string design. The columns “start” and “end” denote the starting and ending position of each identified region in the exemplar strain for Norovirus (see Table 11). The “gene” column provides the official gene symbol for each region, and the “seq” column provides the reference/exemplary strain sequence for that region. The final row of each table, with gene name “full string” gives the GSS linker-linked exemplary string design, with SP domain in the N terminal and MITD domain in the C terminal.

### Example 34: Exemplary Methods for Determination of HLA Binding Peptides (NoV)

Per norovirus clade (as defined herein), the protein sequences of all constituent strains were dissolved into all possible substrings (“candidate HLA binding peptides”) of lengths 8-12. Candidate HLA binding peptides of lengths 8-12 were scored for binding potential to 105 HLA-I alleles using an algorithm called neonmhc1. Candidate HLA binding peptides of lengths 12-20 were scored for binding potential to 85 HLA-II alleles using an algorithm called neonmhc2. Neonmhc1 is a proprietary algorithm based on proprietary data using methods similar to those published in Abelin et al. “Mass spectrometry profiling of HLA-associated peptidomes in mono-allelic cells enables more accurate epitope prediction”, Immunity (2017) 46(2):315-326, the entire contents of which are incorporated herein by reference for purposes described herein. Neonmhc2 is a proprietary algorithm based on proprietary data using methods similar to those published in Abelin et al., “Defining HLA-II ligand processing and binding rules with mass spectrometry enhances cancer epitope prediction” Immunity, (2019) 51(4):766-779.e17, the entire contents of which are incorporated herein by reference for purposes described herein. These two algorithms are part of a larger suite of proprietary analytical tools referred to as “RECON”; neonmhc1 predictions and neonmhc2 predictions are both occasionally referred to as “RECON” predictions herein. Neonmhc1 and neonmhc2 predictions are made on a continuous “percent rank” scale (Nielsen and Andreatta, “NetMHCpan-3.0; improved prediction of binding to MHC class I molecules integrating information from multiple receptor and peptide length datasets” Genome Medicine (2016) 8:33, the entire contents of which are incorporated herein by reference for purposes described herein) where smaller values indicate a higher likelihood of binding. Conservation was determined for each peptide by calculating the percentage of strains with at least one instance of the peptide within their proteome. Thus, a peptide with a conservation of 60% appears is encoded by 60% of the viral strains.

Table 16A (for Norovirus GI), Table 16B (for Norovirus GII.P2), Table 16C (for Norovirus GII.P4), Table 16D (for Norovirus GII.P7), Table 16E (for Norovirus GII.P12), Table 16F (for Norovirus GII.P16), Table 16G (for Norovirus GII.P17), and Table 16H (for Norovirus GIX) each contain peptides grouped according to likelihood of HLA-binding (per allele) and conservation predicted by neonmhc1, wherein sequences of such peptides have been submitted via DVD and are incorporated herein by reference in their entirety. “Very strong” indicates a percent rank binding score less than 0.1%. “Strong” indicates a percent rank binding score between 0.1% and 1%. “Moderate” indicates a percent rank binding score between 1% and 2%. Peptides with weaker predicted binding scores are not included in the table. “High conservation” indicates that the peptide is present in >90% of strains. “Moderate conservation” indicates that the peptide is present in 50%-90% of strains. “Poor conservation” indicates that the peptide is present in <50% of strains.

Table 16I (for Norovirus GI), Table 16J (for Norovirus GII.P2), Table 16K (for Norovirus GII.P4), Table 16L (for Norovirus GII.P7), Table 16M (for Norovirus GII.P12), Table 16N (for Norovirus GII.P16), Table 16O (for Norovirus GII.P17), and Table 16P (for Norovirus GIX) each contain peptides grouped according to likelihood of HLA-binding (per allele) and conservation predicted by neonmhc2, wherein sequences of such peptides have been submitted via DVD and are incorporated herein by reference in their entirety. “Very strong” indicates a percent rank binding score less than 0.5%. “Strong” indicates a percent rank binding score between 0.5% and 2%. “Moderate” indicates a percent rank binding score between 2% and 5%. Peptides with weaker predicted binding scores are not included in the table. “High conservation” indicates that the peptide is present in >90% of strains. “Moderate conservation” indicates that the peptide is present in 50%-90% of strains. “Poor conservation” indicates that the peptide is present in <50% of strains.

HLA binding peptides identified herein (e.g., HLA-I binding peptides and/or HLA-II binding peptides) for each norovirus clade can be delivered by immunogenic compositions described herein. In some embodiments, one or more HLA binding peptides (e.g., HLA-I binding peptides and/or HLA-II binding peptides) selected from Tables 16A-16P herein can be encoded by one or more polynucleotides (e.g., RNA) described herein. In some embodiments, a polynucleotide (e.g., RNA) described herein can encode a polyepitopic polypeptide comprising a plurality of (including, e.g., at least 2, at least 3, at least 4, at least 5, at least 6, at least 7, at least 8, at least 9, at least 10, at least 15, at least 20, at least 25, at least 30, or more) HLA binding peptides (e.g., HLA-I binding peptides and/or HLA-II binding peptides) selected from Tables 16A-16P herein.

### Example 35: Exemplary Methods for Determination of T Cell Epitopes (NoV)

In order to prioritize CD4 and CD8 epitopes for RNA string design for each virus, in some embodiments all known immunogenic and/or HLA-presented epitopes were first considered. Known immunogenic epitopes were curated from IEDB, only considering relevant T-cell epitopes, for example, in some embodiments only CD4 T-cell epitopes were considered for the CD4 shortlist. In some embodiments, B-cell epitopes and MHC ligands from IEDB were omitted. Known HLA-presented epitopes were curated from the HLA ligandomics datasets re-searched via mass spectroscopy (e.g., SpectraMill). Depending on the number of immunogenic and/or HLA-presented epitopes for the given virus and T-cell subset (CD4 or CD8), one of two procedures as described below were followed:
1) If the number of epitopes exceeded the maximum amount of epitopes desired (e.g., 70 for CD4, 100 for CD8), then epitopes were filtered out based on the following criteria, applied procedurally until less than the maximum amount of epitopes desired remained:
   Remove epitopes poorly conserved in viral strains (<50%) & with poor RECON prediction values for any prioritized HLA allele (>2%-rank for CD8, >5%-rank for CD4). A list of prioritized HLA alleles is provided below.
   Remove epitopes moderately conserved in viral strains (<90%)
   Remove epitopes with moderate RECON predictions for any prioritized HLA allele (>1%-rank for CD8, >2%-rank for CD4)
   Remove epitopes with strong RECON predictions for any prioritized HLA allele (>0.1%-rank for CD8, >0.5%-rank for CD4)
2) If the number of epitopes was less than the minimum amount of epitopes desired (e.g., 40 for CD4, 60 for CD8), then epitopes were supplemented using the following logic: for each prioritized HLA allele (e.g., as described below), epitopes were selected based on the strength of RECON predictions. Only epitopes which were highly conserved in viral strains (>90%) were considered. Epitopes were selected until the maximum number of epitopes desired would be exceeded while keeping the number of epitopes selected per allele equal. HLA allele prioritization: The top 5 most frequent HLA alleles by US population (data from Gragert et al., “Six-locus high resolution HLA haplotype frequencies derived from mixed-resolution DNA typing for the entire US donor registry” Human Immunology (2013) 74(10): 1313-1320, the entire contents of which are incorporated herein by reference for the purposes described herein) were selected for this analysis. For Class I (CD8), this included the A, B, and C loci. For Class II (CD4), this included the DRB1, DRB345, DQ, and DP loci. If desirable, additional HLA alleles can be included for analysis.

It is noted that due to significant diversity in Norovirus strains across other genotypes, epitope conservation was calculated in this Example by only considering the Norovirus GII.P4 strains. One of ordinary skill in the art reading the present disclosure will appreciate that such approach can be adapted for any Norovirus genotype with a sufficient number of strains in order to calculate a meaningful conservation metric.

The shortlisted epitopes were designed as RNA strings using approaches described below for HLA-I epitopes and HLA-II epitopes. For HLA-I epitopes, all pairwise epitope orderings were assessed for their likelihood of producing proteasomal cleavage events at the desired position. This was done using a proprietary cleavage predictor trained using proprietary data using methods similar to those presented in Abelin et al. “Mass spectrometry profiling of HLA-associated peptidomes in mono-allelic cells enables more accurate epitope prediction” Immunity (2017) 46(2): 315-326, the entire contents of which are incorporated herein by reference for the purposes described herein. Treating these pairwise scores as a directed graph wherein each epitope represents a node and each cleavage score represents an edge-wait, a heuristic approach was used (see, e.g., Hahsler M, “TSP: Traveling Salesperson Problem (TSP). R package version 1.1-10 (2020), which can be online accessible at https://CRAN.R-project.org/package=TSP; Hahsler M, “TSP-Infrastructure for the traveling salesperson problem” Journal of Statistical Software (2007) 23(2): 1-21, the entire contents each of which are incorporated herein by reference for the purposes described herein) to traverse the graph while maximizing on-target cleavability (noting that while the solution was referred as “optimal”, the algorithm is only a heuristic and not guaranteed to provide the globally optimal solution). Additionally or alternatively, in some embodiments, additional amino-acid linker sequences can be added between epitope pairs as needed to redress any adjacencies that that fail to meet suitable cleavage prediction scores. For HLA-II epitopes, the sequences were concatenated without linkers or special considerations for ordering. However, both approaches for HLA-I epitopes and HLA-II epitopes can be used to meet design objectives (such as reducing the presence of non-target junctional epitopes or achieving desired biophysical properties in the resultant polypeptide).

The top portion of Table 15A contains HLA-I epitopes (e.g., CD8 epitopes) for Norovirus GII.P4 shortlisted for inclusion in a string-of-epitopes vaccine, while the remaining portion of Table 15A contains HLA-II epitopes (e.g., CD4 epitopes) for Norovirus GII.P4 shortlisted for inclusion in a string-of-epitopes vaccine. Epitopes represent a mix of informatic predictions (per RECON) and sequences that have been defined as presented or immunogenic experimentally (per literature).

Table 15B in part shows the amino acid sequence corresponding to a candidate RNA string encoding a sequence of prioritized HLA-I epitopes for Norovirus GII.P4. In some embodiments, the epitope sequences are ordered in a manner that maximizes the likelihood that the target epitopes will be processed and presented (see Methods as described above). In some embodiments, the sequence can additionally or alternatively be designed to minimize non-target junctional epitopes (for example, by choosing an epitope ordering that results epitope adjacencies unlikely to produce peptides that bind common HLAs), to achieve desired conformational properties in the resultant polypeptide (e.g., an enrichment of alpha helices, which could likewise be achieved by selecting an ordering with optimal epitope adjacencies), or some combination of the three objectives described herein. In some embodiments, one or more linker sequences can be additionally or alternatively used to achieve the above-described objectives. In some embodiments, the corresponding nucleotide sequence can be determined by reverse translation using the wildtype viral sequence, codon optimization, and/or some other codon selection scheme that optimizes one or more aspects of the resulting RNA string (e.g., but not limited to stability, the lack of hairpins, etc.).

Table 15B also shows the amino acid sequence corresponding to a candidate RNA string encoding a sequence of prioritized HLA-II epitopes for Norovirus GII.P4. In some embodiments, by permuting the epitopes and/or adding one or more linkers, the sequence could be further optimized to achieve specific features (e.g., lack of non-target junctional epitopes and/or presence of desired polypeptide properties, as described herein). In some embodiments, the corresponding nucleotide sequence can be determined by reverse translation using the wildtype viral sequence, codon optimization, and/or some other codon selection scheme that optimizes one or more aspects of the resulting RNA string (e.g., but not limited to stability, the lack of hairpins, etc.).

### Example 36: Exemplary RNA Constructs Encoding NoV Antigens

The present example describes certain exemplary NoV antigens, and sequences encoding them, that may be utilized in certain embodiments of the present disclosure.

In some embodiments, an antigen delivered in accordance with the present disclosure is or comprises a NoV protein or an immunogenic portion thereof (e.g., comprising one or more epitopes thereof) as indicated in Table 12A or a NoV protein encoded by a gene listed in Tables 13A-13B or an immunogenic portion thereof (e.g., comprising one or more epitopes thereof). In some embodiments, an antigen delivered in accordance with the present disclosure is or comprises one or more regions of one or more NoV proteins, which regions each independently comprise one or more CD4+ epitopes and/or one or more CD8+ epitopes for Norovirus GI as indicated in Tables 14A-14B. In some embodiments, an antigen delivered in accordance with the present disclosure is or comprises one or more regions of one or more NoV proteins, which regions each independently comprise one or more CD4+ epitopes and/or one or more CD8+ epitopes for Norovirus GII.P4 as indicated in Tables 14C-14D. In some embodiments, an antigen delivered in accordance with the present disclosure is or comprises one or more regions of one or more NoV proteins, which regions each independently comprise one or more CD4+ epitopes and/or one or more CD8+ epitopes for Norovirus GII.P7 as indicated in Tables 14E-14F. In some embodiments, an antigen delivered in accordance with the present disclosure is or comprises one or more regions of one or more NoV proteins, which regions each independently comprise one or more CD4+ epitopes and/or one or more CD8+ epitopes for Norovirus GII.P12 as indicated in Tables 14G-14H. In some embodiments, an antigen delivered in accordance with the present disclosure is or comprises one or more regions of one or more NoV proteins, which regions each independently comprise one or more CD4+ epitopes and/or one or more CD8+ epitopes for Norovirus GII.P16 as indicated in Tables 14I-14J. In some embodiments, an antigen delivered in accordance with the present disclosure is or comprises one or more regions of one or more NoV proteins, which regions each independently comprise one or more CD4+ epitopes and/or one or more CD8+ epitopes for Norovirus GII.P17 as indicated in Tables 14K-14L. In some embodiments, an antigen delivered in accordance with the present disclosure is or comprises one or more regions of one or more NoV proteins, which regions each independently comprise one or more CD4+ epitopes and/or one or more CD8+ epitopes for Norovirus GIX as indicated in Tables 14M-14N. In some embodiments, an antigen delivered in accordance with the present disclosure is or comprises a NoV peptide as indicated in one of Table 15A and Tables 16A-16P. In some embodiments, such an antigen is delivered by administration of an RNA encoding such an antigen.

In some particular embodiments, an administered RNA has a structure:
Structure 1: m₂^{7,3′-O}Gppp(m₁^{2′-O})ApG-hAg-Kozak-SEC-Immunogen —FI-A30L70, wherein m₂^{7,3′-O}Gppp(m₁^{2′-O})ApG=5′ cap; hAg=5′ UTR human alpha-globin; SEC=signal peptide (SP); Immunogen=a nucleotide sequence comprising a sequence that encodes an antigen described herein; FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence.

In some embodiments, an administered RNA has a structure:
Structure 2: m₂^{7,3′-O}Gppp(m₁^{2′-O})ApG-hAg-Kozak-SEC-Immunogen-MITD-FI-A30L70, wherein m₂^{7,3′-O}Gppp(m₁^{2′-O})ApG=5′ cap; hAg=5′ UTR human alpha-globin; SEC=signal peptide (SP); Immunogen=a nucleotide sequence comprising a sequence that encodes one or more antigens described herein; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence.

In some embodiments, an Immunogen sequence encodes a plurality of immunogenic fragments (e.g., comprising epitopes) from an antigen. In some embodiments, an Immunogen sequence encodes a plurality of immunogenic fragments (e.g., epitopes) from two or more antigens. In some embodiments. In some embodiments, such immunogenic fragments are linked together to form an Immunogen sequence by linkers (e.g., in some embodiments a linker that is enriched in G and/or S amino acid residues). In some embodiments, a linker may be or comprise an amino acid sequence of GGSGGGGSGG. In some embodiments, a linker may be or comprise an amino acid sequence of GGSLGGGGSG.

### Example 37: Exemplary RNA Constructs Encoding Multiepitope NoV Antigens

The present example describes certain exemplary NoV multiepitope antigens, and sequences encoding them, that may be utilized in certain embodiments of the present disclosure.

### A) Exemplary Construct Encoding a NoV Multi-Epitope Polypeptide #1

Structure: m₂^{7,3′-O}Gppp(m^{2′-O})ApG-hAg-Kozak-SEC-CD8 string-MITD-FI-A30L70, wherein m₂^{7,3′-O}Gppp(m₁^{2′-O})ApG=5′ cap; hAg=5′ UTR human alpha-globin; SEC=signal peptide (SP); CD8 string=a nucleotide sequence comprising a sequence that encodes a plurality of (e.g., two or more) CD8+ T cell epitopes described herein; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence. In some embodiments, the MITD element may be absent.

In some embodiments, a CD8 string may comprise at least 2 (including, e.g., at least 1, at least 2, at least 3, at least 4, at least 5, at least 10, at least 15, at least 20, or more) CD8+ T cell epitopes and/or HLA-I epitopes for norovirus GII.P4 as listed in Table 15A. In some embodiments, a CD8 string may comprise an amino acid sequence as recited in Table 15B. In some embodiments, a CD8 string may comprise at least 2 (including, e.g., at least 1, at least 2, at least 3, at least 4, at least 5, at least 10, at least 15, at least 20, or more) CD8+ T cell epitopes and/or HLA-I epitopes as listed in Tables 16A-16H.

### B) Exemplary Construct Encoding a NoV Multi-Epitope Polypeptide #2

Structure: m₂^{7,3′-O}Gppp(m₁^{2′-O})ApG-hAg-Kozak-SEC-CD4 string-MITD-FI-A30L70, wherein m₂^{7,3′-O}Gppp(m₁^{2′-O})ApG=5′ cap; hAg=5′ UTR human alpha-globin; SEC=signal peptide (SP); CD4 string=a nucleotide sequence comprising a sequence that encodes a plurality of (e.g., two or more) CD4+ T cell epitopes described herein; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence. In some embodiments, the MITD element may be absent.

In some embodiments, a CD4 string may comprise at least 2 (including, e.g., at least 1, at least 2, at least 3, at least 4, at least 5, at least 10, at least 15, at least 20, or more) CD4+ T cell epitopes and/or HLA-II epitopes for norovirus GII.P4 as listed in Table 15A. In some embodiments, a CD4 string may comprise an amino acid sequence as recited in Table 15B. In some embodiments, a CD8 string may comprise at least 2 (including, e.g., at least 1, at least 2, at least 3, at least 4, at least 5, at least 10, at least 15, at least 20, or more) CD8+ T cell epitopes and/or HLA-I epitopes as listed in Tables 16I-16P.

### C) Exemplary Construct Encoding a NoV Multi-Epitope Polypeptide #3

Structure: m₂^{7,3′-O}Gppp(m^{2′-O})ApG-hAg-Kozak-SEC-CD8 chunk sequence-MITD-FI-A30L70, wherein m₂^{7,3′-O}Gppp(m₁^{2′-O})ApG=5′ cap; hAg=5′ UTR human alpha-globin; SEC=signal peptide (SP); CD8 chunk sequence=a nucleotide sequence comprising a sequence that encodes one or more regions of one or more NoV polypeptides, wherein each region predominantly comprises CD8+ epitopes; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence. In some embodiments, the MITD element may be absent.

In some embodiments, a CD8 chunk sequence may comprise at least 2 (including, e.g., at least 1, at least 2, at least 3, at least 4, at least 5, at least 10, at least 15, at least 20, or more) immunogenic fragment(s) of polypeptide(s) encoded by gene(s) or peptide(s) as listed in Table 14A for Norovirus GI; Table 14C for Norovirus GII.P4; Table 14E for Norovirus GII.P7; Table 14G for Norovirus GII.P12; Table 14I for Norovirus GII.P16; Table 14K for Norovirus GII.P17; or Table 14M for Norovirus GIX. In some embodiments, a sequence comprising SEC, CD8 chunk sequence, and MITD is represented by the sequence denoted as “full string” in Table 14A for Norovirus GI; Table 14C for Norovirus GII.P4; Table 14E for Norovirus GII.P7; Table 14G for Norovirus GII.P12; Table 14I for Norovirus GII.P16; Table 14K for Norovirus GII.P17; or Table 14M for Norovirus GIX.

### D) Exemplary Construct Encoding a NoV Multi-Epitope Polypeptide #4

Structure: m₂^{7,3′-O}Gppp(m₁^{2′-O})ApG-hAg-Kozak-SEC-CD4 chunk sequence-MITD-FI-A30L70, wherein m₂^{7,3′-O}Gppp(m₁^{2′-O})ApG=5′ cap; hAg=5′ UTR human alpha-globin; SEC=signal peptide (SP); CD4 chunk sequence=a nucleotide sequence comprising a sequence that encodes one or more regions of one or more NoV polypeptides, wherein each region predominantly comprises CD4+ epitopes; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence. In some embodiments, the MITD element may be absent.

In some embodiments, a CD4 chunk sequence may comprise at least 2 (including, e.g., at least 1, at least 2, at least 3, at least 4, at least 5, at least 10, at least 15, at least 20, or more) immunogenic fragment(s) of polypeptide(s) encoded by gene(s) or peptide(s) as listed in Table 14B for Norovirus GI; Table 14D for Norovirus GII.P4; Table 14F for Norovirus GII.P7; Table 14H for Norovirus GII.P12; Table 14J for Norovirus GII.P16; Table 14L for Norovirus GII.P17; or Table 14N for Norovirus GIX. In some embodiments, a sequence comprising SEC, CD4 chunk sequence, and MITD is represented by the sequence denoted as “full string” in Table 14B for Norovirus GI; Table 14D for Norovirus GII.P4; Table 14F for Norovirus GII.P7; Table 14H for Norovirus GII.P12; Table 14J for Norovirus GII.P16; Table 14L for Norovirus GII.P17; or Table 14N for Norovirus GIX.

### Example 38: Exemplary NoV Peptide String Designs

The present Example exemplifies certain constructs (referred to herein as “strings”) of multiple No V epitopes (which can be from one or more clades as identified herein) linked to one another and useful, for example, in immunogenic compositions (e.g., vaccine compositions) or otherwise as described herein.

Strings described in the present Example are designed to contain specific epitopes of NOV, each of which is disclosed herein and, e.g., is predicted and/or selected as described here, for example through use of an MHC-binding algorithm as described herein. The strings presented in the present Example are designed for therapeutic use in preventing and/or treating NoV infection (and in certain embodiments NoV latent infection) and can be administered as polynucleotide constructs, e.g., mRNA encapsulated in a lipid nanoparticle.

In some embodiments, strings exemplified herein are encoded in an RNA that includes a 5′-UTR and 3′-UTR. Epitopes are interconnected by peptide linkers, encoded by their respective polynucleotide sequences. In some embodiments, one or more linkers may have a specific cleavage site.

Exemplary amino acid sequences for exemplary strings denoted as “full string” in Tables 14A-14N, each of which includes a SP domain, GS-enriched linkers, and a MITD domain. Table 15B shows additional exemplary amino acid sequences for exemplary CD8 string and CD4 strings. In some embodiments, polynucleotide sequences are codon optimized (e.g., for efficient translation in humans).

### Example 39: Exemplary NoV Antigen Identification, Selection and/or Characterization

The present Example describes identification, selection and/or characterization of certain NoV protein sequences useful as or in (i.e., as part of) antigens as described herein.

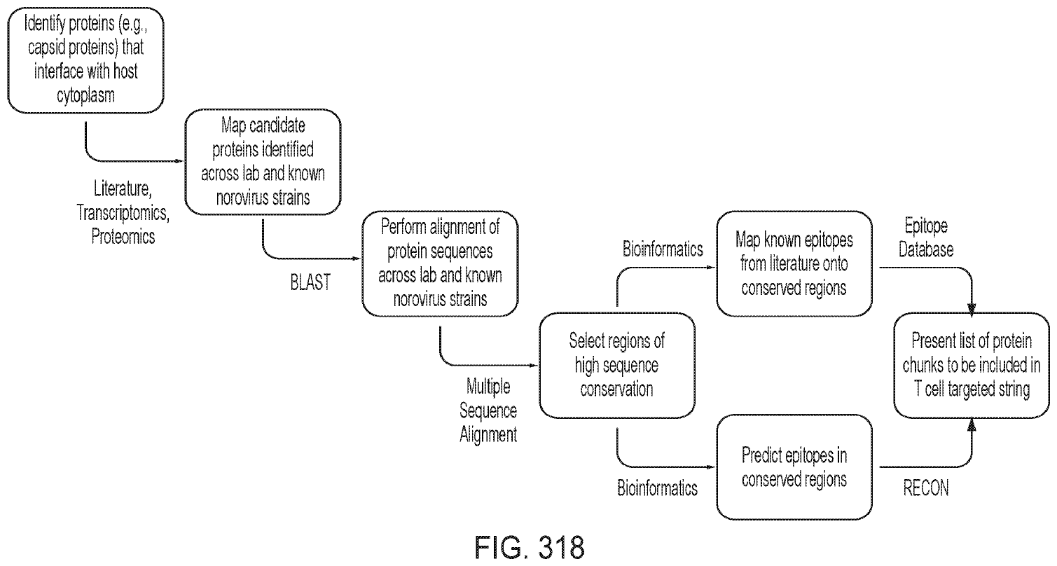

FIG. 318 presents a flow diagram of a process used to identify, characterize, and/or select certain NoV protein sequences (e.g., particular variants) and/or fragments or epitopes thereof, that may be particularly useful in the practice of the present invention. In some embodiments, a utilized antigen is or comprises a NoV protein, or fragment or epitope thereof, as identified or characterized as described in the present Example.

As depicted, viral proteins that include one or more portions expected or known to interface with host cytoplasm are identified, for example by literature review, considering transcriptomic (i.e., RNA expression levels) and/or proteomic (i.e., expressed protein levels) data. In some embodiments, degree of conservation of candidate proteins across relevant NoV strains (e.g., in relevant geographic region) is considered.

Various lab and field isolate strains can be considered for assessing conserved proteins and T cell epitopes. In some embodiments, one or more strains identified in Table 11 was considered for assessing conserved proteins and T epitopes.

Immunogenicity of conserved proteins was also considered, for example by review of literature and/or application of predictive algorithms as described herein.

In accordance with the present example, in some embodiments, each of the NoV proteins encoded by genes shown in Tables 13A-13B and combinations thereof were identified as of particular interest. In some embodiments, one or more of these proteins, or one or more fragments or epitopes thereof is used as or included in an antigen. In some embodiments, a peptide or protein is encoded by a Pro gene, an RdRp gene, or combinations thereof. In some embodiments, a peptide or protein is encoded by a gene selected from: Nterm, Pro, VP1, and combinations thereof.

In some embodiments, an antigen may be or comprise one or more, and specifically may comprise a plurality, of distinct portions (e.g., epitope-containing fragments) of one or more of these proteins, for example in a string construct as described herein. In some embodiments, exemplary CD8 string candidates were designed utilizing one or more epitope-containing fragments of a plurality of proteins encoded by one or more of the following genes: NTPase, Pro, Nterm, and VP1. In some embodiments, representatives of such epitope-containing fragments for norovirus GI are shown in Table 14A and a corresponding exemplary string candidate is shown in Table 14A (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA).

In some embodiments, representatives of such epitope-containing fragments for norovirus GII.P4 are shown in Table 14C and a corresponding exemplary string candidate is shown in Table 14C (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA).

In some embodiments, representatives of such epitope-containing fragments for norovirus GII.P7 are shown in Table 14E and a corresponding exemplary string candidate is shown in Table 14E (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA).

In some embodiments, representatives of such epitope-containing fragments for norovirus GII.P12 are shown in Table 14G and a corresponding exemplary string candidate is shown in Table 14G (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA).

In some embodiments, representatives of such epitope-containing fragments for norovirus GII.P16 are shown in Table 14I and a corresponding exemplary string candidate is shown in Table 14I (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA).

In some embodiments, representatives of such epitope-containing fragments for norovirus GII.P17 are shown in Table 14K and a corresponding exemplary string candidate is shown in Table 14K (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA).

In some embodiments, representatives of such epitope-containing fragments for norovirus GIX are shown in Table 14M and a corresponding exemplary string candidate is shown in Table 14M (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA).

In some embodiments, exemplary CD4 string candidates were designed utilizing one or more epitope-containing fragments of a plurality of proteins encoded by one or more of the following genes: Pro, Nterm, and VP1. In some embodiments, representatives of such epitope-containing fragments for norovirus GI are shown in Table 14B and a corresponding exemplary string candidate is shown in Table 14B (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA).

In some embodiments, representatives of such epitope-containing fragments for norovirus GII.P4 are shown in Table 14D and a corresponding exemplary string candidate is shown in Table 14D (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA).

In some embodiments, representatives of such epitope-containing fragments for norovirus GII.P7 are shown in Table 14F and a corresponding exemplary string candidate is shown in Table 14F (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA).

In some embodiments, representatives of such epitope-containing fragments for norovirus GII.P12 are shown in Table 14H and a corresponding exemplary string candidate is shown in Table 14H (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA).

In some embodiments, representatives of such epitope-containing fragments for norovirus GII.P16 are shown in Table 14J and a corresponding exemplary string candidate is shown in Table 14J (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA).

In some embodiments, representatives of such epitope-containing fragments for norovirus GII.P17 are shown in Table 14L and a corresponding exemplary string candidate is shown in Table 14L (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA).

In some embodiments, representatives of such epitope-containing fragments for norovirus GIX are shown in Table 14N and a corresponding exemplary string candidate is shown in Table 14N (See “Full String”), which include SP domain (MKMRVMAPRTLILLLSGALALTETWAGS), GS-enriched linkers (e.g., GGSGGGGSGG;GGSGGGGSGG; and/or GGSLGGGGSG), and MITD domain (IVGIVAGLAVLAVVVIGAVVATVMCRRKSSGGKGGSYSQAASSDSAQGSDVSLTA).

In some embodiments, a secretory signal (“See”) or a signal peptide (SP) domain present in exemplary string candidates described herein (e.g., a CD8 string and/or a CD4 string as described herein) may be that from HSV-2 gD SP MGRLTSGVGTAALLVVAVGLRVVCA; in alternative embodiments, a different secretory signal, e.g., from HSV-1 gD SP, is used. In some embodiments, a signal peptide may be or comprise an IgE signal peptide. In some embodiments, a signal peptide may be or comprise an IgE HC (Ig heavy chain epsilon-1) signal peptide. In some embodiments, a signal peptide that may be useful in accordance with the present disclosure may comprise one of the following sequences:
a. MDSKGSSQKGSRLLLLLVVSNLLLPQGVVG;
b. MDWTWILFLVAAATRVHS;
c. METPAQLLFLLLLWLPDTTG;
d. MLGSNSGQRVVFTILLLLVAPAYS (Japanese encephalitis PRM signal sequence);
e. MKCLLYLAFLFIGVNCA (VSVg protein signal sequence);
f. MWLVSLAIVTACAGA (Japanese encephalitis JEV signal sequence); or
g. MFVFLVLLPLVSSQC.

In some embodiments, certain chunk boundary considerations are incorporated into the string constructs, for example establishing chunk boundaries to minimize presence of sequences (e.g., epitopes) that may overlap with the human proteome.

### Example 40: Exemplary Vaccine Composition Delivering at Least One NoV Antigen

The present Example describes certain exemplary vaccine compositions.

In some embodiments, a provided vaccine candidate will contain at least 2 RNAs, at least one of which encodes a NoV antigen described herein (e.g., a full length NoV protein or one or more fragments or epitopes thereof, such as a string construct described herein), and optionally at least one of which encodes at least one other conserved NoV protein (or fragment(s) or epitope(s) thereof, such as in a string construct as described herein; in some embodiments such a string construct may include fragments or epitopes from two or more different NoV proteins).

In some embodiments, two or more (e.g., 3, for example 2 of which are/encode NoV antigen string constructs and one of which is/encodes a string construct of a plurality of CD8 and/or CD4 epitopes from other conserved NoV proteins) are formulated together in a single LNP formulation; in other embodiments, individual RNAs may be separately formulated in (the same or different) LNP formulations and such may be mixed together (e.g., in a 1:1 ratio of each RNA, or alternatively in a 1:1 ratio of NoV-antigen-encoding-RNA to “other” RNA so that, for example, for a composition comprising 2 NoV-antigen-encoding RNAs and one other RNA, the ratios would be 0.5:0.5:1).

### Example 41: Exemplary Pre-Clinical Assessment for NoV RNA Vaccine Compositions

The present Example describes certain pre-clinical assessments that may be performed of certain RNA vaccine compositions described herein for treatment and/or prevention of NoV infection and in certain particular embodiments for treatment and/or prevention of NoV latent infection.

In some embodiments, one vaccine candidate is assessed. In some embodiments, more than one different vaccine candidate may be assessed. In some such embodiments, different candidates may vary, for example, in:
a) RNA platform (e.g., unmodified RNA, modified RNA, saRNA);
b) Encoded antigen(s);
c) Number of RNAs;
d) Non-coding elements of RNA construct (e.g., cap and/or cap-adjacent sequences, 5′-UTR, 3′-UTR, and/or PolyA tail); and/or
e) Lipid composition of LNP.

In some embodiments, pre-clinical assessment of certain RNA vaccine compositions (e.g., LNP formulated mRNA-based NoV vaccines) comprises one or more of assessment in challenge experiments, assessment of level of protection, assessment of immunogenicity, and/or assessment of functional antibody responses.

LNP formulated mRNA-based NoV vaccines are tested in a challenge model. Non-human primate models, such as Rhesus macaques and Cynomolgus monkey, and/or rodent models, such as C57/B16 mice, Balb/c mice, NODscidIL2Rγnull mice, or a recombination activation gene (Rag^{-/-}) and common gamma chain (γc^{-/-}) deficient BALB/c mouse; and/or gnotobiotic (Gn) pig models; and/or natural flora piglets; and/or Gn calf models, inoculated with NoV (e.g., by oral infection for larger animals or by intraperitoneal injection for mice models), are administered a first vaccination and can be administered an additional vaccination (e.g., 1, 2, 3, 4, 5, 6, 7, 8, 9, or 10 additional vaccinations) following the first vaccination. One of ordinary skill in the art will appreciate that appropriate animal models, e.g., as described in Todd and Tripp “Human Norovirus: Experimental Models of Infection” Viruses (2019) 11(2):151 (the entire content of which is incorporated herein by reference for the purposes described herein) can be used in a challenge model. Wherein more than one vaccination is administered, the vaccinations are administered at an interval of 1, 2, 3, 4, 5, 6, 7, or 8 week intervals). Following vaccination, animals are challenged by NoV. Animals are then evaluated for reduced viral loads in intestinal lining (e.g., enterocytes). In some embodiments, an animal model is challenged in a plurality of instances (e.g., before first vaccination and/or wherein additional vaccinations are administered, at any time point between or after vaccinations). Following challenge, animals subjected to the study may be assessed according to any method known in the art, including, for example, serology assessment, immunogenicity, level of protection, etc.

In some embodiments, serum antibody characterization and/or serum transfer experiments (e.g., from one vaccinated species to a different non-vaccinated species, e.g., from vaccinated non-human primate to non-vaccinated mouse) are conducted (e.g., to assess protective antibody response).

In some embodiments, certain RNA vaccine compositions of the present disclosure are assessed for level of protection. Level of protection can be assessed according to any suitable method known in the art.

In some embodiments, certain RNA vaccine compositions of the present disclosure are assessed for immunogenicity. For example, ELISA can be used to determine NoV-specific antibody titers and/or avidity of antibodies generated in response to certain RNA vaccine compositions of the present disclosure to NoV antigens (e.g., genogroup- and/or genotype-specific NoV antigens). In some embodiments, serum or mucosal antibody titers against NoV antigens (e.g., genogroup- and/or genotype-specific NoV antigens) is determined by ELISA using standard methods. In some embodiments, for example, ELISpot (e.g., for CD8+, CD4+ T cells and/or IFNγ) and assessment of pro-inflammatory cytokine responses with splenocytes from immunized and/or challenged animal models and peptide pools derived from vaccine targets can also be assessed. In some embodiments, for example, phenotyping of immune responses (e.g., by flow cytometry) are assessed. In some embodiments, for example, T cell depletion and/or protection assays are conducted to assess immunogenicity (e.g., according to any suitable known method in the art).

In some embodiments, one or more functional responses of antibodies generated in response to certain RNA vaccine compositions of the present disclosure are assessed. Functional antibody responses can be assessed, for example, using a NoV neutralization assay. In some embodiments, a NoV in vitro neutralization assay is performed to evaluate one or more anti-NoV antibodies (e.g., antibodies directed to genogroup- and/or genotype-specific NoV antigens) in neutralizing NoV (e.g., certain genogroup- and/or genotype-specific NoV antigens). For example, anti-NoV serum antibodies are obtained by collecting the sera of animals (e.g., mice) vaccinated with NoV mRNA vaccines. NoV virus (e.g., genogroup- and/or genotype-specific NoV) are added to the diluted sera and neutralization is allowed to continue for 1 hour at room temperature. Cells are seeded in 96-wells one day before and the virus/serum mixtures are added to the cells. The cells are fixed on the next day and NoV-specific staining is performed. The plates are scanned and analyzed. A neutralization titer is expressed as the highest dilution required to achieve a 50% reduction in the number of plaques.

In some embodiments, functional antibody responses can be assessed, for example, using passive transfer studies of sera from immunized animals to naïve animals that are challenged and assessing level of protection.

### Example 42: Exemplary Characterization Studies of NoV Vaccine Candidates or Vaccine Compositions

The present Example describes certain potential characterization studies that may be utilized, for example, to identify, select, and/or characterize vaccine candidates or vaccine compositions (e.g., manufacturing batches thereof), or components thereof as described herein.

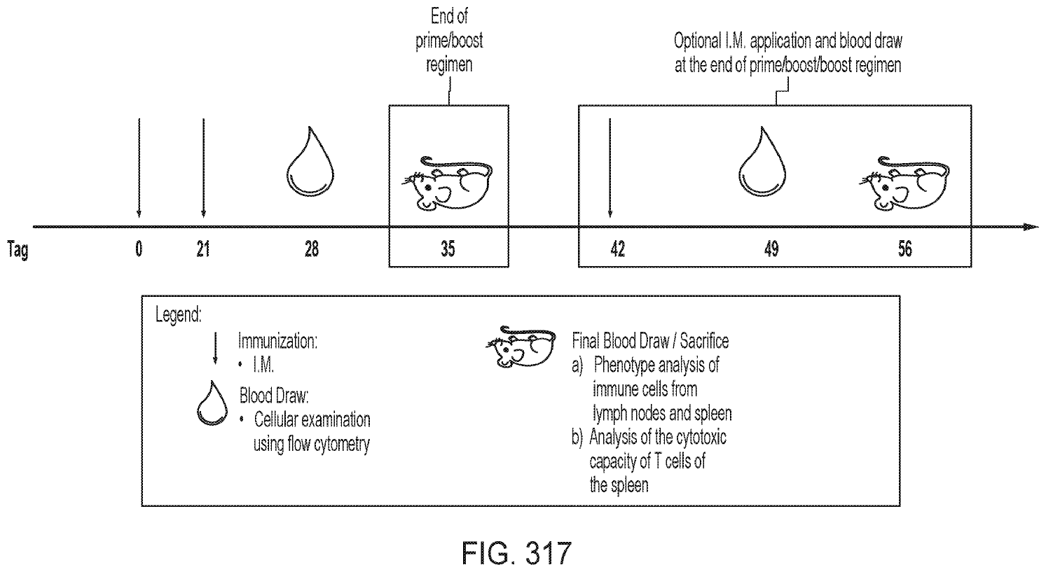

FIG. 317 presents a potential immunization protocol that can be utilized to assess ability of a vaccine candidate that comprises or delivers an antigen(s) as described herein to induce B- and/or T-cells, e.g., after intramuscular immunization, directed to the antigen(s) and/or epitope(s) thereof. In some embodiments, level and/or diversity of response is determined. In some embodiments, presence and/or level of neutralizing antibodies is/are determined. In some embodiments, protection of the immunized subject from challenge with NoV is assessed.

Alternatively or additionally, in some embodiments, one or more in vitro assessments may be performed, for example:
(1) in vitro expression of an antigen encoded by an RNA included in a vaccine composition; and/or
(2) in vitro potency of antigen expressed from an RNA included in a vaccine composition as described herein.

### Example 43: Exemplary Clinical Studies of NoV RNA Vaccine Compositions

The present Example describes certain clinical assessments that may be performed of certain RNA vaccine compositions described herein for treatment and/or prevention of NoV infection and in certain particular embodiments for treatment and/or prevention of NoV latent infection.

In some embodiments, more than one different vaccine candidate may be assessed. In some such embodiments, different candidates may vary, for example in:
(1) RNA platform (e.g., unmodified RNA, nucleoside-modified RNA, self-amplifying RNA (saRNA), trans-amplifying RNA);
(2) encoded antigen—e.g.,
   which NoV protein(s) utilized
   full length protein antigen vs fragment vs plurality of fragments vs fusion with one or more heterologous sequences (e.g., membrane tether, secretion, linker(s))
   epitopes from different (and/or multiple) phases of NoV life cycle
(3) number of RNAs
(4) non-coding elements of RNA construct
   cap and/or cap-adjacent sequences
   5′ UTR
   3′ UTR
   polyA tail
(5) lipid composition of LNP

In one particular embodiment, up to three candidate vaccines that have only 1 mRNA encoding for a NoV protein (e.g., genogroup- and/or genotype-specific NoV protein and/or any NoV protein described in Tables 12A-12D) or variant are evaluated and/or up to three candidates that contain 2 mRNAs, one encoding for a NoV protein (e.g., genogroup- and/or genotype-specific NoV protein and/or any NoV protein described in Tables 12A-12D) or variants and another one encoding for CD8 and/or CD4 epitopes from conserved antigens (and optionally considering conserved T-cell epitopes from various stages of the NoV life cycle) are evaluated. In this particular exemplary embodiment, vaccine candidates may be evaluated by intramuscular administration, for example, based on a dose-escalation scheme.

### Example 44: Exemplary LNP Formulations

The present Example describes certain preferred LNP formulations useful for vaccine compositions (e.g., RNA vaccine compositions) as described herein.

In some embodiments, LNP formulations that are useful for vaccine compositions as described herein can comprise at least one ionizable aminolipid. In some embodiments, LNP formulations that are useful for vaccine compositions as described herein can comprise at least one cationically ionizable aminolipid. In some embodiments, LNP formulations that are useful for vaccine compositions as described herein can further comprise a helper lipid, which in some embodiments may be or comprise a neutral helper lipid. In some embodiments, LNP formulations that are useful for vaccine compositions as described herein can further comprise a polymer-conjugated lipid, for example in some embodiments PEG-conjugated lipids. In some embodiments, LNP formulations that are useful for vaccine compositions as described herein can comprise at least one ionizable aminolipid, at least one helper lipid (e.g., a neutral helper lipid, which in some embodiments may comprise a phospholipid, a steroid, or combinations thereof), and at least one polymer-conjugated lipid (e.g., PEG-conjugated lipid). In some embodiments, an exemplary LNP formulation may comprise an ionizable aminolipid, a phospholipid, a steroid, and a PEG-conjugated lipid.

In some embodiments, an ionizable aminolipid may be present in an LNP formulation within a range of 45 to 55 mol percent, 40 to 50 mol percent, 41 to 49 mol percent, 41 to 48 mol percent, 42 to 48 mol percent, 43 to 48 mol percent, 44 to 48 mol percent of total lipids. In some embodiments, an exemplary ionizable aminolipid is or comprises ((4-hydroxybutyl)azanediyl)bis(hexane-6,1-diyl)bis(2-hexyldecanoate) (also known as 6-[N-6-(2-hexyldecanoyloxy)hexyl-N-(4-hydroxybutyl)amino]hexyl 2-hexyldecanoate). In some embodiments, an exemplary ionizable aminolipid is or comprises SM-102 (heptadecan-9-yl 8 ((2 hydroxyethyl)(6 oxo 6-(undecyloxy)hexyl)amino)octanoate) or an aminolipid as described in Sabnis et al. “A Novel Amino Lipid Series for mRNA Delivery: Improved Endosomal Escape and Sustained Pharmacology and Safety in Non-human Primates” Mol. Ther. (2018) 26:1509-1519. In some embodiments, an exemplary ionizable aminolipid is or comprises an ionizable aminolipid as disclosed in US2020/0163878 or WO2018/078053, the entire contents of each of which are incorporated herein by reference for the purposes described herein.

In some embodiments, a phospholipid may be present in an LNP formulation within a range of 5 to 15 mol percent, 7 to 13 mol percent, or 9 to 11 mol percent of total lipids. In some embodiments, an exemplary phospholipid is or comprises 1,2-Distearoyl-sn-glycero-3-phosphocholine (DSPC).

In some embodiments, a sterol may be present in an LNP formulation within a range of 30 to 50 mol percent, 35 to 45 mol percent or 38 to 43 mol percent of total lipids. In some embodiments, an exemplary sterol is or comprises cholesterol.

In some embodiments, a polymer conjugated lipid (e.g., PEG-conjugated lipid) may be present in an LNP formulation within a range of 1 to 10 mol percent, 1 to 5 mol percent, or 1 to 2.5 mol percent of total lipids. In some embodiments, an exemplary PEG-conjugated lipid is or comprises 2-[(polyethylene glycol)-2000]—N,N-ditetradecylacetamide (also known as 2-[2-(ω-methoxy (polyethyleneglycol2000) ethoxy]-N,N-ditetradecylacetamide). In some embodiments, an exemplary phospholipid is or comprises PEG2000-DMG (1-monomethoxypolyethyleneglycol-2,3-dimyristylglycerol with polyethylene glycol of average molecular weight 2000). In some embodiments, an exemplary PEG-conjugated lipid is or comprises a PEG-lipid as disclosed in US2020/0163878 or WO2018/078053, the entire contents of each of which are incorporated herein by reference for the purposes described herein.

In some embodiments, an exemplary LNP formulation comprises (i) an ionizable aminolipid within a range of 45 to 55 mol percent of total lipids; (ii) a phospholipid within a range of 8 to 12 mol percent of total lipids; (iii) a steroid within a range of 35 to 45 mol percent of total lipids; and (iv) a polymer conjugated (e.g., PEG-conjugated polymer) within a range of 1 to 2 mol percent of total lipids; and RNA molecules as described herein that are encapsulated within or associated with the lipid nanoparticles.

In some embodiments, an exemplary LNP formulation comprises (i) ionizable amino lipid within a range of 45 to 55 mol percent of total lipids; (ii) DSPC within a range of 5 to 15 mol percent of total lipids; (iii) cholesterol within a range of 35 to 45 mol percent of total lipids; and (iv) a PEG-conjugated lipid within a range of 1 to 2 mol percent of total lipids; and RNA molecules as described herein that are encapsulated within or associated with the lipid nanoparticles.

In some embodiments, an exemplary LNP formulation comprises (i) an ionizable aminolipid within a range of 40 to 50 mol percent of total lipids; (ii) a phospholipid within a range of 5 to 15 mol percent of total lipids; (iii) a steroid within a range of 35 to 45 mol percent of total lipids; and (iv) a polymer conjugated (e.g., PEG-conjugated polymer) within a range of 1 to 10 mol percent of total lipids; and RNA molecules as described herein that are encapsulated within or associated with the lipid nanoparticles. In some such embodiments, an ionizable aminolipid is or comprises ((4-hydroxybutyl)azanediyl)bis(hexane-6,1-diyl)bis(2-hexyldecanoate) (also known as 6-[N-6-(2-hexyldecanoyloxy)hexyl-N-(4-hydroxybutyl)amino]hexyl 2-hexyldecanoate). In some such embodiments, a phospholipid is or comprises 1,2-Distearoyl-sn-glycero-3-phosphocholine (DSPC). In some such embodiments, a steroid is or comprises cholesterol. In some such embodiments, a polymer conjugated polymer is or comprises 2-[(polyethylene glycol)-2000]—N,N-ditetradecylacetamide (also known as 2-[2-(ω-methoxy (polyethyleneglycol2000) ethoxy]-N,N-ditetradecylacetamide).

In one embodiment, an exemplary LNP formulation comprises the following lipids included in Table 26 below and RNA molecules as described herein.

TABLE 26
Exemplary LNP Formulation

| | Proportion |
|---|---|
| Lipids | (mol %) |
| ((4-hydroxybutyl)azanediyl)bis(hexane-6,1-diyl)bis(2- | 47-49 |
| hexyldecanoate) (also known as 6-[N-6-(2- | (e.g., 47.5) |
| hexyldecanoyloxy)hexyl-N-(4-hydroxybutyl)amino]hexyl 2- | |
| hexyldecanoate) | |
| 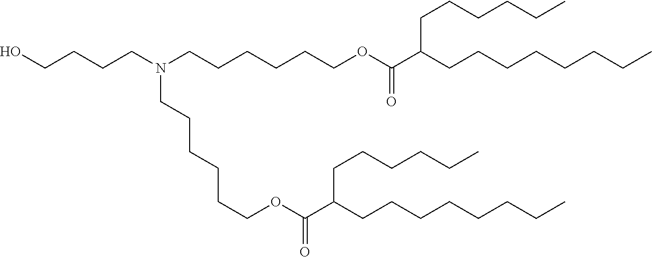 [SMILES: CCCCCCCCC(CCCCCC)C(=O)OCCCCCCN(CCCCO)CCCCCCOC(=O)C(CCCCCC)CCCCCCCC] | |
| 2-[(polyethylene glycol)-2000]-N,N-ditetradecylacetamide | 1.5-2.0 |
| (also known as 2-[2-(ω-methoxy(polyethyleneglycol2000) | (e.g., 1.8) |
| ethoxy]-N,N-ditetradecylacetamide) | |
| 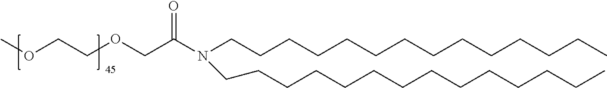 | |
| DSPC | 9.5-10.5 |
| | (e.g., 10) |
| Cholesterol, synthetic | 39-42 |
| | (e.g., 40.7) |

In some embodiments, an exemplary LNP formulation comprises an ionizable aminolipid, DSPC, cholesterol, and PEG-conjugated lipid at a molar ratio of approximately 50:10:38.5:1.5 or 47.5:10:40.8:1.7. In some embodiments, an ionizable amino lipid is or comprises ((4-hydroxybutyl)azanediyl)bis(hexane-6,1-diyl)bis(2-hexyldecanoate) (also known as 6-[N-6-(2-hexyldecanoyloxy)hexyl-N-(4-hydroxybutyl)amino]hexyl 2-hexyldecanoate).

In some embodiments, an exemplary LNP formulation comprises (i) SM-102 (heptadecan-9-yl 8 ((2 hydroxyethyl)(6 oxo 6-(undecyloxy)hexyl)amino)octanoate) within a range of 45 to 55 mol percent of total lipids; (ii) DSPC within a range of 5 to 15 mol percent of total lipids; (iii) cholesterol within a range of 35 to 45 mol percent of total lipids; and (iv) PEG2000-DMG within a range of 1 to 2 mol percent of total lipids; and RNA molecules as described herein that are encapsulated within or associated with the lipid nanoparticles.

In some embodiments, an exemplary LNP formulation comprises (i) ((4-hydroxybutyl)azanediyl)bis(hexane-6,1-diyl)bis(2-hexyldecanoate) (also known as 6-[N-6-(2-hexyldecanoyloxy)hexyl-N-(4-hydroxybutyl)amino]hexyl 2-hexyldecanoate) within a range of 45 to 55 mol percent of total lipids; (ii) DSPC within a range of 5 to 15 mol percent of total lipids; (iii) cholesterol within a range of 35 to 45 mol percent of total lipids; and (iv) a PEG-conjugated lipid within a range of 1 to 2 mol percent of total lipids; and RNA molecules as described herein that are encapsulated within or associated with the lipid nanoparticles.

### Example 45: Exemplary Prediction and/or Characterization of MHC Presentation

The present Example describes an exemplary approach to assessing MHC presentation which may be used in accordance with the present disclosure to select and/or characterize antigenic peptides as described herein (e.g., from VZV, CMV, NoV, or a latent virus known in the art, e.g., ones described herein).

In some embodiments, an antigenic peptide is selected and/or characterized through analysis of its amino acid sequence using an MHC-peptide presentation prediction algorithm or MHC-peptide presentation predictor, for example implemented in a computer processor (e.g., a computer processor that has been trained by a machine learning software), which determines a likelihood of binding and presentation of an epitope by an MHC class I or an MHC class II antigen.

In some embodiments, an MHC-peptide presentation prediction algorithm or MHC-peptide presentation predictor is or comprises neonmhc 1 and/or neonmhc2, which predict and/or characterize likelihood of MHC class I and MHC class II binding, respectively. Alternatively or additionally, in some embodiments, an MHC-peptide presentation prediction algorithm or MHC-peptide presentation predictor is or comprises NetMHCpan or NetMHCIIpan. In some embodiments, a hidden markov model approach may be utilized for MHC-peptide presentation prediction and/or characterization. In some embodiments, the peptide prediction model MARIA may be utilized. In some embodiments, NetMHCpan is not utilized to predict or characterize likelihood of MHC binding for peptides as described herein. In some embodiments, the peptide prediction model MARIA may be utilized. In some embodiments, NetMHCIIpan is not utilized to predict or characterize likelihood of MHC binding for peptides as described herein. In some embodiments, neither NetMHCpan nor NetMHCIIpan is utilized to predict or characterize likelihood of MHC binding for peptides as described herein. In some embodiments, an MHC-peptide presentation prediction algorithm or MHC-peptide presentation predictor is or comprises RECON, which offers high quality MHC-peptide presentation prediction based on expression, processing and binding capabilities.

In some embodiments, multiple MHC-peptide presentation prediction algorithms or MHC-peptide presentation predictors may be utilized; in some such embodiments, results obtained with different strategies are compared with one another. In some embodiments, a determination that a particular peptide is likely to be or is significantly likely to be presented by MHC class I or MHC class II may be considered to be better established if two or more algorithms or predictors agree.

Alternatively or additionally, identification and/or characterization of MHC binding (e.g., of MHC class I and/or MHC class II binding) may involve experimental assessment, or reports thereof, which may involve presentation in one or more in vitro systems and/or in one or more organisms. In some embodiments, such assessment utilizes mammalian cells or systems; in some embodiments such assessment utilizes primate (e.g., in some embodiments, human and/or in some embodiments, non-human primate) cells or systems.

### Example 46: Exemplary HLA Class I and Class I Binding Assays

The present Example describes exemplary techniques for assessing peptide binding to HLA molecules. In some embodiments, exemplified technologies may determine and/or characterize (e.g., quantify) binding affinities for HLA class I and HLA class II.

In general, binding assays can be performed with peptides that are either motif-bearing or not motif-bearing. A detailed description of an exemplary protocol that can be utilized to measure the binding of peptides to Class I and Class II MHC has been published (Sette et al., Mol. Immunol. 31:813, 1994; Sidney et al., in Current Protocols in Immunology, Margulies, Ed., John Wiley & Sons, New York, Section 18.3, 1998). Briefly, purified MHC molecules (5 to 500 nM) are incubated with various unlabeled peptide inhibitors and 1-10 nM ¹²⁵I-radiolabeled probe peptides for 48h in PBS containing 0.05% Nonidet P40 (NP40) (or 20% w/v digitonin for H-2 IA assays) in the presence of a protease inhibitor cocktail. Assays are typically performed at pH 7.0, though in some embodiments a lower pH (typically above about pH 4.0) may be performed.

Following incubation, MHC-peptide complexes are separated from free peptide, for example by gel filtration, e.g., on 7.8 mm×15 cm TSK200 columns (TosoHaas 16215, Montgomeryville, PA), though those skilled in the art will appreciate that column size can be adjusted, if desired, for example to improve separation of bound vs unbound peptides of a particular size or characteristic. The eluate from the TSK columns is passed through a Beckman 170 radioisotope detector, and radioactivity is plotted and integrated using a Hewlett-Packard 3396A integrator, and the fraction of peptide bound is determined.

Radiolabeled peptides can be iodinated using the chloramine-T method. Typically, in preliminary experiments, each MHC preparation is titered in the presence of fixed amounts of radiolabeled peptides to determine the concentration of HLA molecules necessary to bind 10-20% of the total radioactivity. Subsequent inhibition and direct binding assays can be performed using these HLA concentrations.

Since under these conditions [label]<[HLA] and IC₅₀>[HLA], the measured IC5o values are often reasonable approximations of the true K_{D} values. Peptide inhibitors are typically tested at concentrations ranging from 120 μg/ml to 1.2 ng/ml, and are tested in two to four completely independent experiments. To allow comparison of the data obtained in different experiments, a relative binding figure is typically calculated for each peptide by dividing the IC₅₀ of a positive control for inhibition by the IC₅₀ for each tested peptide (typically unlabeled versions of the radiolabeled probe peptide). For database purposes, and inter-experiment comparisons, relative binding values can be compiled. Such values can subsequently be converted back into IC₅₀ nM values, for example by dividing the IC₅₀ nM of the positive controls for inhibition by the relative binding of the peptide of interest. This method of data compilation has proven to provide accurate and consistent comparison for peptides that have been tested on different days, or with different lots of purified MHC.

Alternatively or additionally, live cell/flow cytometry-based assays can also be used. This is a well-established assay utilizing the TAP-deficient hybridoma cell line T2 (American Type Culture Collection (ATCC Accession No. CRL-1992), Manassas, Va.). The TAP deficiency in this cell line leads to inefficient loading of MHCI in the ER and an excess of empty MHCIs. Salter and Cresswell, EMBO J. 5:94349 (1986); Salter, Immunogenetics 21:235-46 (1985). Empty MHCIs are highly unstable, and are therefore short-lived. When T2 cells are cultured at reduced temperatures, empty MHCIs appear transiently on the cell surface, where they can be stabilized by the exogenous addition of MHCI-binding peptides. To perform this binding assay, peptide-receptive MHCIs are induced by culturing aliquots of 10⁷ T2 cells overnight at 26° C. in serum free AIM-V medium alone, or in medium containing escalating concentrations (0.1 to 100 μM) of peptide. Cells are then washed twice with PBS, and subsequently incubated with a fluorescent tagged HLA-A02:01-specific monoclonal antibody, BB7.2, to quantify cell surface expression. Samples are acquired on a FACS Calibur instrument (Becton Dickinson) and the mean fluorescence intensity (MFI) determined using the accompanying Cellquest software.

### Example 47: Confirmation of Immunogenicity

The present Example describes an exemplary method for confirmation of immunogenicity, in particular by utilizing in vitro expansion (IVE) assays to test the ability of one or more antigens or peptides (e.g., from VZV, CMV, NoV, or a latent virus known in the art, e.g., ones described herein) to expand CD8+ T cells.

Mature professional APCs are prepared for these assays in the following way. 80-90×10⁶ PBMCs from a healthy human donor are plated in 20 ml of RPMI media containing 2% human AB serum, and incubated at 37° C. for 2 hours to allow for plastic adherence by monocytes. Non-adherent cells are removed and the adherent cells are cultured in RPMI, 2% human AB serum, 800 IU/ml of GM-CSF and 500 IU/ml of IL-4. After 6 days, TNF-alpha is added to a final concentration of 10 ng/ml. On day 7, the dendritic cells (DC) are matured either by the addition of 12.5 mg/ml poly I:C or 0.3 μg/ml of CD40L. The mature dendritic cells (mDC) are harvested on day 8, washed, and either used directly or cryopreserved for future use.

For the IVE of CD8+ T cells, aliquots of 2×10⁵ mDCs are pulsed with each peptide at a final concentration of 100 micromole, incubated for 4 hours at 37° C., and then irradiated (2500 rads). The peptide-pulsed mDCs are washed twice in RPMI containing 2% human AB serum. 2×10⁵ mDCs and 2×10⁶ autologous CD8+ cells are plated per well of a 24-well plate in 2 ml of RPMI containing 2% human AB, 20 ng/ml IL-7 and 100 μg/ml of IL-12, and incubated for 12 days. The CD8+ T cells are then re-stimulated with peptide-pulsed, irradiated mDCs. Two to three days later, 20 IU/ml IL-2 and 20 ng/IL7 are added. Expanding CD8+ T cells are re-stimulated every 8-10 days, and are maintained in media containing IL-2 and IL-7. Cultures are monitored for peptide-specific T cells using a combination of functional assays and/or tetramer staining. Parallel IVES with the modified and parent peptides allowed for comparisons of the relative efficiency with which the peptides expanded peptide-specific T cells.

### Example 48: Quantitative and Functional Assessment of CD8+ and CD4+ T Cells

The present Example describes exemplary methods to test the ability of one or more antigens or peptides (e.g., from VZV, CMV, NoV, or a latent virus known in the art, e.g., ones described herein) to induce CD8+ and/or CD4+ T cell responses.

### Tetramer Staining

MHC tetramers are purchased or manufactured on-site, and are used to measure peptide-specific T cell expansion in the IVE assays. For the assessment, tetramer is added to 1×10⁵ cells in PBS containing 1% FCS and 0.1% sodium azide (FACS buffer) according to manufacturer's instructions. Cells are incubated in the dark for 20 minutes at room temperature. Antibodies specific for T cell markers, such as CD8, are then added to a final concentration suggested by the manufacturer, and the cells are incubated in the dark at 4° C. for 20 minutes. Cells are washed with cold FACS buffer and resuspended in buffer containing 1% formaldehyde. Cells are acquired on a FACS Calibur (Becton Dickinson) instrument, and are analyzed by use of Cellquest software (Becton Dickinson). For analysis of tetramer positive cells, the lymphocyte gate is taken from the forward and side-scatter plots. Data are reported as the percentage of cells that are CD8+/Tetramer+.

CD4⁺ T cell responses towards antigens or peptides can be tested using the ex vivo induction protocol. In this example, CD4+ T cell responses are identified by monitoring IFNγ and/or TNFα production in an antigen specific manner.

### Evaluation of Antigen Presentation:

For a subset of antigens or peptides (e.g., that are or comprise predicted or selected epitope(s) as described herein), affinity for the indicated HLA alleles and/or stability with the HLA alleles can be determined.

An exemplary detailed description of a protocol that can be utilized to measure the binding affinity of peptides to Class I MHC has been published (Sette et al, Mol. Immunol. 31(11):813-22, 1994). In brief, MHCI complexes are prepared and bound to radiolabeled reference peptides. Peptides are incubated at varying concentrations with these complexes for 2 days, and the amount of remaining radiolabeled peptide bound to MHCI is measured using size exclusion gel-filtration. The lower the concentration of test peptide needed to displace the reference radiolabeled peptide demonstrates a stronger affinity of the peptide for MHCI. Peptides with affinities to MHCI <50 nM are generally considered strong binders while those with affinities <150 nM are considered intermediate binders and those <500 nM are considered weak binders (Fritsch et al, 2014).

An exemplary detailed description of a protocol that can be utilized to measure binding stability of peptides to Class I MHC has been published (Harndahl et al. J Immunol Methods. 374:5-12, 2011). Briefly, synthetic genes encoding biotinylated MHC-I heavy and light chains are expressed in E. coli and purified from inclusion bodies using standard methods. The light chain (β2m) is radio-labeled with iodine (125I), and combined with the purified MHC-I heavy chain and peptide of interest at 18° C. to initiate pMHC-I complex formation. These reactions are carried out in streptavidin coated microplates to bind the biotinylated MHC-I heavy chains to the surface and allow measurement of radiolabeled light chain to monitor complex formation. Dissociation is initiated by addition of higher concentrations of unlabeled light-chain and incubation at 37° C. Stability is defined as the length of time in hours it takes for half of the complexes to dissociate, as measured by scintillation counts. MHC-II binding affinity with peptides is measured following the same general procedure as with measuring MHCI-peptide binding affinity. Prediction algorithms utilized for predicting MHCII alleles for binding to a given peptide are described herein. Alternatively or additionally, NetMHCIIpan may be utilized for prediction of binding.

To assess whether particular peptides or epitopes could be processed and presented from a larger polypeptide context, peptides eluted from HLA (class I or class II) molecules isolated from cells expressing the genes of interest can be analyzed by tandem mass spectrometry (MS/MS).

### ELISPOT

Peptide-specific T cells are functionally enumerated, for example, using the ELISPOT assay (BD Biosciences), which measures the release of IFNgamma from T cells on a single cell basis. Target cells (T2 or HLA-A0201 transfected C₁Rs) are pulsed with 10 uM peptide for 1 hour at 37° C., and washed three times. 1×10⁵ peptide-pulsed targets are co-cultured in the ELISPOT plate wells with varying concentrations of T cells (5×10² to 2×10³) taken from the IVE culture. Plates are developed according to the manufacturer's protocol, and analyzed on an ELISPOT reader (Cellular Technology Ltd.) with accompanying software. Spots corresponding to the number of IFNgamma-producing T cells are reported as the absolute number of spots per number of T cells plated. T cells expanded on modified peptides are tested not only for their ability to recognize targets pulsed with the modified peptide, but also for their ability to recognize targets pulsed with the parent peptide.

### CD107 Staining

CD107a and b are expressed on the cell surface of CD8+ T cells following activation with cognate peptide. The lytic granules of T cells have a lipid bilayer that contains lysosomal-associated membrane glycoproteins (“LAMPs”), which include the molecules CD107a and b. Without wishing to be bound by any one theory, it is proposed that, when cytotoxic T cells are activated through the T cell receptor, the membranes of these lytic granules mobilize and fuse with the plasma membrane of the T cell. The granule contents are released, and this leads to the death of the target cell. As the granule membrane fuses with the plasma membrane, C107a and b are exposed on the cell surface, and therefore are markers of degranulation. Because degranulation as measured by CD107a and b staining is reported on a single cell basis, the exemplary assay is used to functionally enumerate peptide-specific T cells. To perform the assay, peptide is added to HLA-A0201-transfected cells C1R to a final concentration of 20 μM, the cells are incubated for 1 hour at 37° C., and washed three times. 1×10⁵ of the peptide-pulsed CIR cells are aliquoted into tubes, and antibodies specific for CD107a and b are added to a final concentration suggested by the manufacturer (Becton Dickinson). Antibodies are added prior to the addition of T cells in order to “capture” the CD107 molecules as they transiently appear on the surface during the course of the assay. 1×10⁵ T cells from the culture are added next, and the samples are incubated for 4 hours at 37° C. The T cells are further stained for additional cell surface molecules such as CD8 and acquired on a FACS Calibur instrument (Becton Dickinson). Data is analyzed using the accompanying Cellquest software, and results are reported as the percentage of CD8+CD107a and b+ cells.

### CTL Lysis

Cytotoxic activity can be measured, for example, using a chromium release assay. Target T2 cells are labeled for 1 hour at 37° C. with Na⁵¹Cr and washed 5×10³ target T2 cells are then added to varying numbers of T cells from the IVE culture. Chromium release is measured in supernatant harvested after 4 hours of incubation at 37° C. The percentage of specific lysis is calculated as:

(Experimental release−spontaneous release)/(Total release−spontaneous release)×100.

### Example 49: Administration of Polyepitopic Compositions

The present Example describes exemplary administration of compositions that comprise or deliver a plurality of epitopes, which can be from a single viral polypeptide described herein or from two or more viral polypeptides (e.g., encoded by two or more distinct genes) described herein. In some embodiments, viral polypeptides are from VZV, CMV, NoV, or a latent virus known in the art, e.g., ones described herein.

For example, a polyepitopic vaccine (e.g., that comprises or delivers a collection of epitopes—e.g., as individual discrete peptides or as one or more polyepitopic peptides or polypeptides such as one or more string constructs as described herein).

In some embodiments, a polyepitopic vaccine comprises or delivers multiple CD4+ and/or CD8+ T cell epitopes. In some embodiments, a polyepitopic vaccine comprises or delivers multiple cytotoxic T lymphocyte (CTL) and/or helper T lymphocyte (ITL) epitopes. In some embodiments, such a vaccine is administered to subject(s) at risk of or having experienced exposure to a viral infection described herein. In some embodiments, such a vaccine is administered to subject(s) at risk of developing a latent viral infection.

In some embodiments, a polyepitopic vaccine as described herein comprises or delivers one or more polypeptides, each of which encompasses multiple epitopes. In some embodiments, one or more monoepitopic peptides or polyepitopic antigens is delivered to a subject by administration of one or more a nucleic acid (e.g., DNA or and RNA) constructs. In some embodiments, a single nucleic acid construct (e.g., a DNA or RNA encoding a polyepitopic antigen) is administered. In some embodiments, a plurality of nucleic acids (e.g., each encoding a different monoepitopic or polyepitopic antigens) is administered. In some embodiments, an administered nucleic acid is an RNA (e.g., an mRNA); in some embodiments, a nucleic acid (e.g., an RNA) is administered in an LNP composition.

In some embodiments, an administered composition includes a pharmaceutically-acceptable excipient, including, e.g., but not limited to an aqueous carrier and/or an adjuvant (e.g., alum).

In some embodiments, an initial administration is followed by one or more booster doses. In some embodiments, a booster dose includes the same amount of polyepitopic construct as the initial dose. In some embodiments, a booster dose include more or less of a polyepitopic construct than was provided in the initial dose. In some embodiments, a booster dose is administered after an interval of at least 1, 2, 3, 4, 5, 6, 7, 8, 9, 12, 11, 12, 13, 14, 15, 16, 17, 18, 29, 20, 21, 22, 23, 24 weeks or more after an initial dose. In some embodiments, a booster dose is administered after an interval of at least 1, 2, 3, 4, 5, 6, 7, 8, 9, 12, 11, 12 months or more after an initial dose. In some embodiments, multiple booster doses are administered. In some embodiments, each subsequent booster is administered at an interval that is the same as or longer than that between its immediate predecessor dose and the dose before that. In some embodiments, 2, 3, 4, 5, 6, or more boosters are administered. In some embodiments, not more than 4 doses total are administered. In some embodiments, not more than 3 doses total are administered. In some embodiments, not more than two doses total are administered. In some embodiments, not more than 1, 2, 3 or 4 doses are administered within a particular 12 month period. In some embodiments, not more than 3, not more than 2, or not more than 1 dose(s) is/are administered within a particular 12 month period (e.g., within 12 months of the initial dose).

In some embodiments, evaluation of an induced immune response (e.g., of magnitude, character, and/or diversity of immune response, such as antibody and/or T cell response) is performed before and/or after one or more doses (e.g., 1, 2, 3, 4, or more weeks after administration of a particular dose and/or within 6, 5, 4, 3, 2, or 1 month of administration of a particular dose) and may, for example be considered in determination of whether one or more booster doses should be administered and/or timing of such booster dose administration. In some embodiments, assessment of an immune response may utilize, for example, techniques that determine presence and/or level of epitope-specific T cell populations (e.g., CTL populations) in a PBMC sample.

### Example 50: Administration of Dendritic Cells

The present Example describes exemplary dendritic cell compositions that comprise or deliver antigens as described herein. In some embodiments, antigens are from VZV, CMV, NoV, or a latent virus known in the art, e.g., ones described herein.

In this example, peptides comprising epitopes as described herein (e.g., identified, designed, selected and/or characterized as described herein) are loaded onto dendritic cells. Peptide-pulsed dendritic cells can be administered to a subject. In some embodiments, such administration may stimulate a T cell response (e.g., a CD4+ and/or CD8+ T cell response) in vivo. In some embodiments, such administration may stimulate a CTL response in vivo.

In this particular Example, dendritic cells (e.g., autologous dendritic cells) are isolated, expanded, and pulsed with peptide epitopes (e.g., T cell epitopes) as described herein. Dendritic cells may then be infused back into the patient. In some embodiments, such infusion can elicit CD4+ and/or CD8+ T cell responses in vivo. In some embodiments, such infusion can elicit CTL and/or HTL responses in vivo. The induced CTL and HTL then destroy (CTL) or facilitate destruction (HTL) of target cells that bear the proteins from which the epitopes in the vaccine are derived.

In some embodiments, ex vivo CTL or HTL responses to a particular antigen can be induced by incubating in tissue culture the patient's, or genetically compatible, CTL or HTL precursor cells together with a source of antigen-presenting cells, such as dendritic cells, and the appropriate immunogenic peptides.

After an appropriate incubation time (typically about 7-28 days), in which the precursor cells are activated and expanded into effector cells, the cells are infused back into the patient, where they will destroy (CTL) or facilitate destruction (HTL) of their specific target cells, i.e., cells displaying relevant epitopes.

### Example 51: Administration of Epitope Binding Agents

The present Example describes administration of epitope binding agents, as an alternative or complement to vaccination strategies describes herein. In some embodiments, epitopes of viral polypeptides are from VZV, CMV, NoV, or a latent virus known in the art, e.g., ones described herein.

For example, among other things, the present disclosure provides technologies for identification and/or characterization of antigens that are particularly amenable to targeting in order to disrupt one or more features of infection. In many embodiments described herein, the present disclosure provides technologies that involve administration or delivery of antigens that are or comprise such epitopes, for example, in order to induce an immune response targeting such epitope(s) in a recipient.

Alternatively or additionally, the present disclosure provides and encompasses technologies for developing, characterizing, and/or administering agents that bind to such epitopes. In some embodiments, such strategies may provide or represent therapeutic interventions, for example useful in addition or as an alternative to vaccination strategies.

Epitope binding agents, such as antibody agents, TCR agents, CAR agents, and/or cells expressing any of the foregoing can be administered in accordance with methodologies known in the art and/or described herein.

In some embodiments, a relevant epitope binding agent can be delivered by administration of a composition that is or comprises the polypeptide binding agent. Alternatively or additionally, in some embodiments, a relevant epitope binding agent can be delivered by administration of a composition that is or comprises a polynucleotide (e.g., a DNA or RNA and, in many favored embodiments, an RNA) encoding the polypeptide binding agent. In some embodiments, a polypeptide binding agent is delivered by administered by a cell (or population thereof) that comprises or expresses the polypeptide and/or a polynucleotide that encodes it.

### Example 52: Exemplary Identification and/or Characterization of Variant Sequences with Immunogenic Potential

The present Example describes technologies for identification and/or characterization of peptide sequences (e.g., from VZV, CMV, NoV, or a latent virus known in the art, e.g., ones described herein) that differ from a relevant reference, for assessment of immunogenic potential.

The full-length amino acid sequence of a variant protein (e.g., as observed in a circulating strain or developed through a predictive model) was derived.

Constituent 9-mer and 10-mer peptide fragments of the variant protein are each scored for binding potential on common HLA alleles (including, e.g., but not limited to HLA-A01:01, HLA-A02:01. HLA-A03:01, HLA-A24:02, HLA-B07:02, and HLA-B08:01) using available algorithms. Peptides scoring better than 1000 nM are noted as potential candidates.

Alternatively or additionally, constituent 9-mer or 10-mer peptide sequences not found in the reference protein sequence are flagged and scored for binding potential on common HLA alleles (including, e.g., but not limited to HLA-A01:01, HLA-A02:01. HLA-A03:01, HLA-A24:02, HLA-B07:02, and HLA-B08:01) using available algorithms.

### Example 53: Exemplary Production, Characterization, and or Use of Certain Polyepitopic Vaccine Compositions

In some embodiments, immunogenicity of a multi-epitopic RNA or polypeptide (wherein epitopes are epitopes from viral polypeptides of VZV, CMV, NoV, or a latent virus known in the art, e.g., ones described herein) is tested in rodents (e.g., mice, e.g., transgenic mice) to evaluate the magnitude of immune response induced against the epitopes tested. In some embodiments, immunogenicity of encoded epitopes in vivo can be correlated with in vitro responses of specific CTL lines against target cells expressing the multi-epitope polypeptides. Thus, in some embodiments, such exemplary experiments can show that a multi-epitopic construct serves to both: 1) generate a cell mediated and/or humoral response and 2) that the induced immune cells recognized cells expressing the encoded epitopes.

In some embodiments, for example, to create a DNA sequence encoding the selected multi-epitope construct (e.g., DNA or RNA) for expression in human cells, amino acid sequences of epitopes to be included can be reverse translated. A human codon usage table can be used to guide the codon choice for each amino acid.

In some embodiments, epitope-encoding DNA sequences are directly adjoined, so that when transcribed and translated, a continuous polypeptide sequence is created.

In some embodiments, expression and/or immunogenicity is optimized. In some such embodiments, expression and/or immunogenicity is optimized by incorporating additional elements into an encoding construct. Without limitation, examples of amino acid sequences that can be reverse translated and included in a multi-epitopic construct sequence include, for example: HLA class I epitopes, HLA class II epitopes, an ubiquitination signal sequence, and/or an endoplasmic reticulum targeting signal. In some embodiments, HLA presentation of CTL and HTL epitopes can be improved by including synthetic (e.g., poly-alanine) or naturally-occurring flanking sequences adjacent to the CTL or HTL epitopes; larger peptides comprising the epitope(s) are within the scope of the present disclosure.

In some embodiments, a multi-epitope-encoding DNA sequence can be produced by assembling oligonucleotides that encode the plus and minus strands of the construct. In some embodiments, overlapping oligonucleotides (e.g., 30-100 bases long) can be synthesized, phosphorylated, purified and annealed under appropriate conditions using suitable technique known in the art. In some embodiments, ends of utilized oligonucleotides can be joined, for example, using ligation (e.g., T4 DNA ligation). In some embodiments, synthetic constructs encoding a multi-epitopic can then be cloned into a desired expression vector (e.g., using a suitable cloning technique known in the art).

In some embodiments, standard regulatory sequences well known to those of skill in the art (e.g., promoters, enhancers, etc.) can be included to ensure expression of a polyepitopic construct in target cells. In some embodiments, for example, a promoter with a down-stream cloning site for insertion of the polyepitopic construct coding sequence; a polyadenylation signal for efficient transcription termination; an E. coli origin of replication; and an E. coli selectable marker (e.g., ampicillin or kanamycin resistance). In some embodiments, a utilized promoter or promoters is not limited and can be used for this purpose, e.g., the human cytomegalovirus (hCMV) promoter. See, e.g., U.S. Pat. Nos. 5,580,859 and 5,589,466 for other suitable promoter sequences.

In some embodiments, vector modifications are used to optimize expression and/or immunogenicity. In some embodiments, introns are utilized for efficient gene expression, and one or more synthetic or naturally-occurring introns are incorporated into the transcribed region. In some embodiments, inclusion of stabilization sequences (e.g., mRNA stabilization sequences) and/or sequences for replication in mammalian cells are used for increasing expression.

In some embodiments, once an expression vector is selected, a multi-epitopic construct coding sequence is cloned into a polylinker region downstream of a promoter (e.g., generating a “plasmid”). In some embodiments, such a plasmid is transformed into an appropriate E. coli strain, and DNA is prepared using any suitable technique known in the art. In some embodiments, the orientation and DNA sequence of the multi-epitopic encoding sequence, as well as all other elements included in the vector, are confirmed using, for example, restriction mapping and/or DNA sequence analysis. In some embodiments, bacterial cells comprising a desired plasmid can be stored, for example, as a master cell bank and/or a working cell bank.

In some embodiments, immunomodulatory sequences contribute to the immunogenicity, e.g., of nucleic acid vaccine constructs. In some embodiments, such sequences are included in a vector, outside the coding sequence, if desired to enhance immunogenicity. In some embodiments, such sequences are immunostimulatory. In some embodiments, such sequences are ISSs or CpGs.

In some embodiments, a bi-cistronic expression vector which allows production of both multi-epitopic construct and a second protein (e.g., included to enhance or decrease immunogenicity) are used. Without limitation, examples of proteins or polypeptides that can enhance the immune response if co-expressed with a multi-epitopic construct include cytokines (e.g., IL-2, IL-12, GM-CSF), cytokine-inducing molecules (e.g., LeIF), costimulatory molecules, or for HTL responses, pan-DR binding proteins. In some embodiments, helper (HTL) epitopes are fused and/or linked to intracellular targeting signals and expressed separately from expressed CTL epitopes; this allows direction of the HTL epitopes to a cell compartment different than that of the CTL epitopes. If required, this could facilitate more efficient entry of HTL epitopes into the HLA class II pathway, thereby improving HTL induction. In contrast to HTL or CTL induction, specifically decreasing the immune response by co-expression of immunosuppressive molecules (e.g. TGF-β) can be beneficial in certain diseases.

In some embodiments, commercially-relevant quantities of plasmid DNA (e.g., for administration or for production of RNA and/or protein for administration) can be produced, for example, by fermentation in E. coli, followed by purification. In some embodiments, aliquots from a working cell bank are used to inoculate growth medium, and grown to a predetermined level (e.g., saturation) in flasks (e.g., shaker flasks) or a bioreactor according to well-known techniques. In some embodiments, plasmid DNA is purified using standard bioseparation technologies such as, for example, solid phase anion-exchange resins supplied by QIAGEN, Inc. (Valencia, California). In some embodiments, supercoiled DNA is separated from open circular and linear forms using gel electrophoresis or other suitable methods known in the art.

In some embodiments, purified plasmid DNA is prepared for injection into a subject using a variety of formulations. In some embodiments, lyophilized DNA is reconstitution in sterile phosphate-buffer saline (PBS). This approach, known as “naked DNA,” and is currently being used for intramuscular (IM) administration in clinical trials. In some embodiments, to maximize the immunotherapeutic effects of polyepitopic vaccine compositions, an alternative method for formulating nucleic acids (e.g., purified plasmid DNA, in vitro transcribed RNA, etc) can be used. A variety of methods have been described, and new techniques can become available. In some embodiments, cationic lipids are used in the formulation (see, e.g., as described by WO 93/24640; Mannino & Gould-Fogerite, BioTechniques 6(7): 682 (1988); U.S. Pat. No. 5,279,833; WO 91/06309; and Felgner, et al., Proc. Nat'l Acad. Sci. USA 84:7413 (1987). In some embodiments, glycolipids, fusogenic liposomes, peptides and compounds referred to collectively as protective, interactive, non-condensing compounds (PINC) are complexed to purified plasmid DNA to influence variables such as stability, intramuscular dispersion, or trafficking to specific organs or cell types.

In some embodiments, a polynucleotide is introduced into cells by use of high-speed cell deformation. During high-speed deformation, cells are squeezed such that temporary disruptions occur in the cell membrane, thus allowing the nucleic acid to enter the cell. In some embodiments, polypeptides are produced from expression vectors, e.g., in a bacterial expression vector, for example, and the proteins can then be delivered to the cell.

In some embodiments, target cell sensitization is used as a functional assay for expression and HLA class I presentation of encoded CTL epitopes. For example, in some embodiments, a polynucleotide is introduced into a mammalian cell line that is suitable as a target for standard CTL chromium release assays. In some embodiments, a transfection method used is dependent on the final formulation. In some embodiments, electroporation is used, e.g., for “naked” polynucleotides (e.g., DNA). In some embodiments, wherein cationic lipids are utilized, direct in vitro transfection is utilized as a transfection method. In some embodiments, a plasmid expressing marker protein or polypeptide (e.g., green fluorescent protein (GFP)) is co-transfected to allow enrichment of transfected cells (e.g., using fluorescence activated cell sorting (FACS)). In some embodiments, cells are then chromium-51 (⁵¹-Cr) labeled and used as target cells for epitope-specific CTL lines; cytolysis, detected by ⁵¹Cr release, indicates both production of, and HLA presentation of encoded CTL epitopes. In some such embodiments, expression of HTL epitopes can be evaluated in an analogous manner using assays to assess HTL activity.

In some embodiments, in vivo immunogenicity is utilized for functional testing. In some embodiments, rodents (e.g., mice, e.g., transgenic mice expressing appropriate human HLA proteins) are immunized with a polyepitopic vaccine composition (e.g., comprising a DNA or RNA active agent). In some embodiments, dose and route of administration are formulation dependent (e.g., IM for DNA in PBS or LNP-formulated DNA or RNA, intraperitoneal (IP) for lipid-complexed DNA). In some embodiments, for example, twenty-one days after immunization, splenocytes are harvested and restimulated for 1 week in the presence of peptides encoding each epitope being tested. Thereafter, for CTL effector cells, assays are conducted for cytolysis of peptide-loaded, ⁵¹Cr-labeled target cells using standard techniques. Lysis of target cells that are sensitized by HLA loaded with peptide epitopes, corresponding to minigene-encoded epitopes, demonstrates vaccine function for in vivo induction of CTLs. Immunogenicity of HTL epitopes is evaluated in transgenic mice in an analogous manner.

### Example 54: Blocking Assays

The present Example describes an exemplary HBGA blocking assay, adapted from Reek et al., J Infect. Dis 202:1212, 2010, that, in some embodiments, can be used to assess or characterize compositions as described herein.

In particular, HBGA blocking assays as described herein can assess ability of serum antibodies to inhibit NoV virus-like particles (VLP) binding to H type 1 or H type 3 synthetic carbohydrates.

Reagents and test samples can be diluted in 0.1 mol/L sodium phosphate buffer (pH, 6.4) with 0.25% fatty acid-free bovine serum albumin (Sigma-Aldrich), and assessed in reactions, for example, that may have a volume of about 100 μL.

NoV VLPs (0.32 μg/mL) for the blocking assays can be produced using a baculovirus expression system, and can be incubated with an equal volume of serum that had been serially 2-fold diluted from the starting dilution (1:25); in some embodiments, incubation is performed for 1 h at 4° C.

In some embodiments, neutravidin-coated, 96-well microtiter plates (Pierce Thermofisher Scientific) are washed and then coated with 2.5 μg/mL of either synthetic polyvalent Lewis d (H type 1)-polyacrylamide (PAA)-biotin or polyvalent (H type 3)-PAA-biotin (Glycotech), e.g., for 1 h at 22° C. Plates are washed (e.g., multiple times) between each incubation step, for example with 0.1 mol/L sodium phosphate buffer (pH, 6.4). The serum-VLP solutions are added and incubated, e.g., at 4° C. for 2 h. Plates are washed, and NoV-specific rabbit polyclonal serum (dilution, 1:5000) and incubated, e.g., for 1 h at 4° C. Incubated plates are again washed, and the incubated with horseradish peroxidase-conjugated, goat antirabbit immunoglobulin G (dilution, 1:5000; Sigma), e.g., for 1 h at 4° C. Color is developed by adding tetramethylbenzidine peroxidase liquid substrate (Kirkegaard and Perry Laboratory) and stopped, e.g., after 10 min of incubation at 22° C., by adding 1 mol/L phosphoric acid.

Optical density (OD) can be measured at 450 nm.

Blank wells incubated with buffer instead of serum-VLP can be used as negative controls, and VLP binding to carbohydrates in the absence of a serum sample can be used as a positive control.

A 50% blocking titer (BT⁵⁰) is defined as the titer at which the OD reading (after subtraction of the blank) is 50% of the OD of the positive control.

In some embodiments, blocking titers >25 are considered indicative of protection. In some embodiments, blocking titers of at least 1:200 are considered to be indicative of protection.

In some embodiments, specificity of observed blocking may be assessed, for example, using the same assay except that, after the plates are coated with carbohydrates, serum samples are incubated directly on the plate without first preincubating with VLP. After washing, VLPs are incubated on the plate and detected as for the blocking assay.

### EXEMPLARY EMBODIMENTS

### Exemplary Viral Related Embodiments

Exemplary embodiments as described below are also within the scope of the present disclosure:
1. A composition for delivery of one or more viral antigens to a subject.
2. A composition comprising one or more RNA molecules that collectively encode one or more viral antigens.
3. The composition of embodiment 1 or 2, wherein at least one RNA molecule of the one or more RNA molecules encodes a single viral antigen.
4. The composition of any one of embodiments 1-3, wherein at least one RNA molecule of the one or more RNA molecules encodes multiple viral antigens.
5. The composition of any one of embodiments 1-4, wherein all of the one or more RNA molecules encode multiple viral antigens.
6. The composition of any one of embodiments 3-5, wherein the one or more RNA molecules encoding multiple viral antigens further comprise a linker.
7. The composition of any one of embodiments 1-6, wherein the one or more RNA molecules comprises a 5′ cap or 5′ cap analog.
8. The composition of embodiment 7, wherein the 5′ cap analog is or comprises Cap0, a Cap1 or a Cap2.
9. The composition of embodiment 7 or 8, wherein a 5′-cap analog is or comprises m₂^{7,3′-O}Gppp(m₁^{2′-O})ApG.
10. The composition of any one of embodiments 1-9, wherein the one or more RNA molecules comprises a sequence encoding a signal peptide.
11. The composition of any one of embodiments 1-10, wherein the one or more RNA molecules comprise at least one non-coding regulatory element.
12. The composition of any one of embodiments 1-11, wherein the one or more RNA molecules comprises a poly-adenine tail.
13. The composition of embodiment 12, wherein the poly-adenine tail is or comprises a modified adenine sequence.
14. The composition of embodiment 12 or 13, wherein the poly-adenine tail comprises at least 100 A nucleotides.
15. The composition of any one of embodiments 12-14, wherein the poly-adenine tail is an interrupted sequence of A nucleotides.
16. The composition according to embodiment 15, wherein the poly-adenine tail comprises 30 adenine nucleotides followed by 70 adenine nucleotides, wherein the 30 adenine nucleotides and 70 adenine nucleotides are separated by a linker sequence.
17. The composition of any one of embodiments 1-16, wherein the one or more RNA molecules comprises at least one 5′ untranslated region (UTR) and/or at least one 3′ UTR.
18. The composition according to embodiment 17, wherein the at least one 5′-UTR is or comprises a modified human alpha-globin 5′-UTR.
19. The composition according to embodiment 17 or 18, wherein the at least one 3′-UTR is or comprises a first sequence from the “amino terminal enhancer of split” (AES) messenger RNA and a second sequence from the mitochondrial encoded 12S ribosomal RNA.
20. The composition of any one of embodiments 1-19, wherein the RNA is a modified RNA, which is modified by substitution of some or all uridine residues with a modified uridine residue.
21. The composition of any one of embodiments 1-20, wherein the RNA is modified by N1-methyl-pseudouridine substitution of some or all uridine residues.
22. The composition of one of embodiments 1-21, wherein the RNA is formulated in lipid nanoparticles comprising a cationic or cationically ionizable lipid, a sterol, a neutral lipid, and a lipid conjugate.
23. The composition of one of embodiments 1-22, wherein the RNA is formulated in lipid nanoparticles comprising a cationically ionizable lipid, a phospholipid, a cholesterol, and a polyethylene glycol (PEG)-lipid.
24. The composition of embodiment 22 or 23, wherein the cationic lipid or cationically ionizable is or comprises ((4-hydroxybutyl)azanediyl)bis(hexane-6,1-diyl)bis(2-hexyldecanoate), the sterol is or comprises a cholesterol, the neutral lipid is or comprises a phospholipid, and the lipid conjugate is or comprises a polyethylene glycol (PEG)-lipid.
25. The composition according to any one of embodiments 22-24, wherein the one or more lipid nanoparticles comprise:
   a. ((4-hydroxybutyl)azanediyl)bis(hexane-6,1-diyl)bis(2-hexyldecanoate);
   b. a cholesterol;
   c. distearoylphosphatidylcholine (DSPC); and
   d. 2-[(polyethylene glycol)-2000]—N,N-ditetradecylacetamide.
26. The composition of embodiment 22 or 23, wherein the cationically ionizable lipid has the following structure:

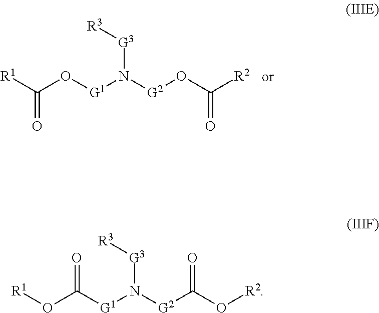

or a pharmaceutically acceptable salt, tautomer or stereoisomer thereof, wherein:
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl
27. The composition of embodiment 26, wherein R¹ or R², or both, has one of the following structures:

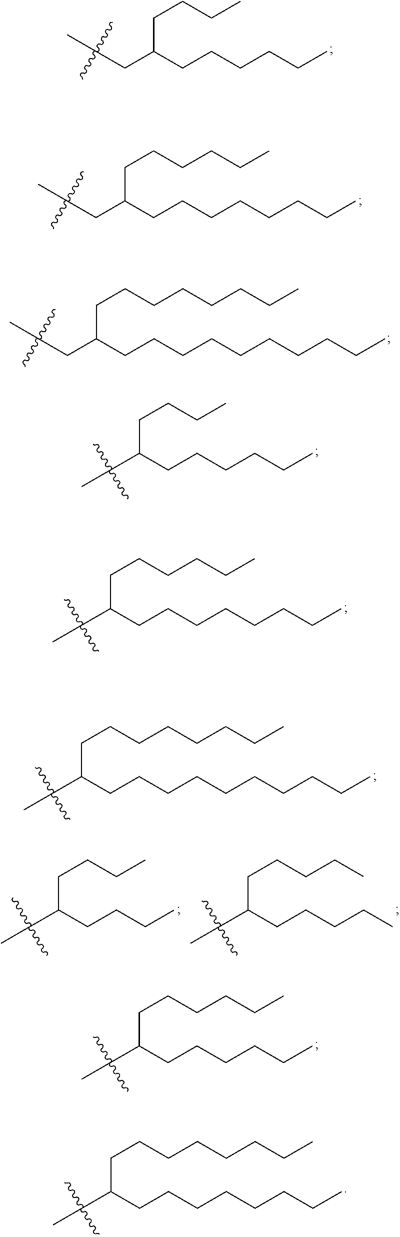

28. The composition of embodiment 26, wherein the cationically ionizable lipid has the following structure:

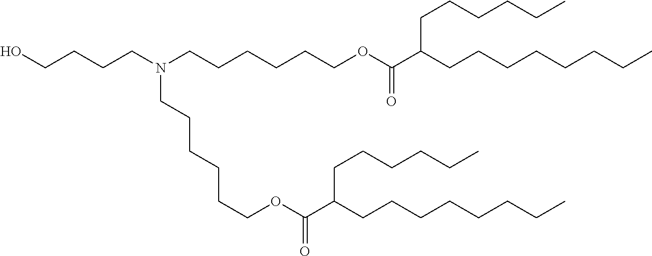 [SMILES: CCCCCCCCC(CCCCCC)C(=O)OCCCCCCN(CCCCO)CCCCCCOC(=O)C(CCCCCC)CCCCCCCC]

29. The composition of any one of embodiments 23-28, wherein the phospholipid is or comprises distearoylphosphatidylcholine (DSPC).
30. The pharmaceutical composition any one of embodiments 23-29, wherein the (PEG)-lipid is or comprises 2-[(polyethylene glycol)-2000]—N,N-ditetradecylacetamide.
31. The composition of any one of embodiments 23-30, wherein the phospholipid is present in a concentration ranging from 5 to 15 mol percent of the total lipids.
32. The composition of any one of embodiments 22-31, wherein the cationically ionizable lipid is present in a concentration ranging from 40 to 55 mol percent of the total lipids.
33. The composition of any one of embodiments 23-32, wherein the cholesterol is present in a concentration ranging from 30 to 50 mol percent of the total lipids.
34. The composition of any one of embodiments 23-32, wherein the (PEG)-lipid is present in a concentration ranging from 1 to 10 mol percent of the total lipids.
35. The composition of any one of embodiments 23-34, wherein the lipid nanoparticles comprise from 40 to 55 mol percent of a cationically ionizable lipid; from 5 to 15 mol percent of a phospholipid; from 30 to 50 mol percent of a cholesterol; and from 1 to 10 mol percent of a PEG-lipid.
36. The composition of embodiment 43, wherein ((4-hydroxybutyl)azanediyl)bis(hexane-6,1-diyl)bis(2-hexyldecanoate) is within a range of about 40 to about 55 mole percent, a cholesterol is within a range of about 30 to about 50 mole percent, distearoylphosphatidylcholine (DSPC) is within a range of about 5 to about 15 mole percent, and 2-[(polyethylene glycol)-2000]—N,N-ditetradecylacetamide is within a range of about 1 to about 10 mole percent.
37. The composition of any one of embodiments 1-36, further comprising at least one salt and/or a cryoprotectant, wherein the cryoprotectant is or comprises sucrose.
38. The composition of any one of embodiments 1-37, wherein the total RNA is present in an amount within a range of 1 ug to 100 ug per dose in the composition.
39. A pharmaceutical composition comprising a composition of any one of embodiments
1-38. 40. The pharmaceutical composition of embodiment 39, which is in a liquid formulation.
41. The pharmaceutical composition of embodiment 39, which is in a frozen formulation.
42. The pharmaceutical composition of embodiment 41, wherein the frozen formulation comprises PBS.
43. The pharmaceutical composition of any one of embodiments 39-42, wherein the composition is formulated for intramuscular administration.
44. The pharmaceutical composition of any one of embodiments 39-42, wherein the composition is formulated for intravenous administration.
45. The pharmaceutical composition of any one of embodiments 39-42, wherein the composition is formulated for subcutaneous administration.
46. A method comprising administering at least one dose of a pharmaceutical composition of any one of embodiments 39-45 to a subject.
47. The method of embodiment 46, wherein the subject is human.
48. The method of embodiment 46 or 47, wherein the subject is suffering from a viral infection.
49. The method of embodiment 46 or 47, wherein the subject intends to be present within a geographical region that has a high viral prevalence within the next three months.
50. The method of embodiment 49, wherein a high viral prevalence is greater than 10% of the population.
51. The method of any one of embodiments 46-50, wherein the subject has previously been treated for a viral infection with a different pharmaceutical composition.
52. The method of any one of embodiments 46-51, further comprising administering a second dose of the pharmaceutical composition to the patient.
53. The method of any one of embodiments 46-52, further comprising administering at least two doses of the pharmaceutical composition to the patient.
54. The method of any one of embodiments 46-53, further comprising administering at least three doses of the pharmaceutical composition to the patient.
55. The method of any one of embodiments 46-54, wherein the method is a method of inducing an anti-viral infection immune response in the subject.
56. The method of embodiment 55, wherein the immune response in the subject comprises an adaptive immune response.
57. The method of embodiment 55 or 56, wherein the immune response in the subject comprises a T-cell response.
58. The method of embodiment 57, wherein the T-cell response is or comprises a CD4+ response.
59. The method of embodiment 57 or 58, wherein the T-cell response is or comprises a CD8+ response.
60. The method of any one of embodiments 55-59, wherein the immune system response comprises a B-cell response.
61. The method of any one of embodiments 55-60, wherein the immune system response comprises the production of antibodies directed against the one or more viral antigens.
62. The pharmaceutical composition of any one of embodiments 39-45 for use in the treatment of a viral infection.
63. The pharmaceutical composition of any one of embodiments 39-45 for use in inducing an anti-viral infection immune response in the subject.
64. Use of the pharmaceutical composition of any one of embodiments 39-45 in the treatment of viral infection.
65. Use of the pharmaceutical composition of any one of embodiments 39-45 in inducing an anti-viral infection immune response in the subject.
66. A polypeptide comprising one or more of viral antigens.
67. A polynucleotide encoding one or more of viral antigens.
68. The polynucleotide of embodiment 67, wherein the polynucleotide is DNA or RNA.
69. A cell comprising a polypeptide of embodiment 66 and/or a polynucleotide of embodiments 67 or 68.
70. A cell comprising a polynucleotide of any one of embodiments 67 or 68.
71. The cell of embodiment 70, wherein the cell expresses the one or more viral antigens encoded by the polynucleotide.

### Exemplary VZV Related Embodiments

Exemplary embodiments as described below are also within the scope of the present disclosure:
1. A composition for delivery of one or more VZV antigens to a subject.
2. A composition comprising one or more RNA molecules that collectively encode one or more VZV antigens.
3. The composition of embodiment 1 or 2, wherein the one or more VZV antigens comprise one or more VZV antigens listed in Table 3A, Table 3B, Table 4A, and/or Tables
5A-5B herein or comprise one or more VZV antigens encoded by respective genes listed in Tables 1A-1I and/or Tables 2A-2B herein.
4. The composition of any one of embodiments 1-3, wherein the one or more RNA molecules encode one or more amino acid sequences found in Table 3A, Table 3B, Table
4A, Table 4B, Table 5A, and/or Table 5B herein.
5. The composition of any one of embodiments 1-4, wherein the one or more RNA molecules comprise one or more sequences found in Table 4A and/or Table 4B herein.
6. The composition of any one of embodiments 1-5, wherein at least one RNA molecule of the one or more RNA molecules encodes a single VZV antigen.
7. The composition of any one of embodiments 1-6, wherein at least one RNA molecule of the one or more RNA molecules encodes multiple VZV antigens.
8. The composition of embodiment 7, wherein the at least one RNA molecule has a sequence found in Table 4A and/or Table 4B herein.
9. The composition of any one of embodiments 1-5, 7 or 8, wherein all of the one or more RNA molecules encode multiple VZV antigens.
10. The composition of embodiment 9, wherein the one or more RNA molecules each have a sequence found in Table 4A and/or Table 4B herein.
11. The composition of embodiment 9 or 10, wherein the one or more RNA molecules each have a sequence found in Table 4A and/or Table 4B herein, and the one or more RNA molecules comprise RNA molecules having different sequences.
12. The composition of any one of embodiments 8-11, wherein the one or more RNA molecules encoding multiple VZV antigens further comprise a linker.
13. The composition of embodiment 12, wherein the linker has a sequence found in Table 22 herein.
14. The composition of any one of embodiments 1-13, wherein the one or more RNA molecules comprises a 5′ cap or 5′ cap analog.
15. The composition of embodiment 14, wherein the 5′ cap analog is or comprises Cap0, a Cap1 or a Cap2.
16. The composition of embodiment 14 or 15, wherein a 5′-cap analog is or comprises m₂^{7,3′-O}Gppp(m₁^{2′-O})ApG.
17. The composition of any one of embodiments 1-16, wherein the one or more RNA molecules comprises a sequence encoding a signal peptide.
18. The composition of embodiment 17, wherein the signal peptide is or comprises a sequence found in Table 20 herein.
19. The composition of embodiment 17, wherein the one or more RNA molecules comprise a sequence encoding a signal peptide found in Table 21 herein.
20. The composition of any one of embodiments 1-19, wherein the one or more RNA molecules comprise at least one non-coding regulatory element.
21. The composition of any one of embodiments 1-20, wherein the one or more RNA molecules comprises a poly-adenine tail.
22. The composition of embodiment 21, wherein the poly-adenine tail is or comprises a modified adenine sequence.
23. The composition of embodiment 21 or 22, wherein the poly-adenine tail comprises at least 100 A nucleotides.
24. The composition of any one of embodiments 21-23, wherein the poly-adenine tail is an interrupted sequence of A nucleotides.
25. The composition according to embodiment 24, wherein the poly-adenine tail comprises 30 adenine nucleotides followed by 70 adenine nucleotides, wherein the 30 adenine nucleotides and 70 adenine nucleotides are separated by a linker sequence.
26. The composition of any one of embodiments 1-25, wherein the one or more RNA molecules comprises at least one 5′ untranslated region (UTR) and/or at least one 3′ UTR.
27. The composition according to embodiment 26, wherein the at least one 5′-UTR is or comprises a modified human alpha-globin 5′-UTR.
28. The composition according to embodiment 26 or 27, wherein the at least one 3′-UTR is or comprises a first sequence from the “amino terminal enhancer of split” (AES) messenger RNA and a second sequence from the mitochondrial encoded 12S ribosomal RNA.
29. The composition of any one of embodiments 1-28, wherein the RNA is a modified RNA, which is modified by substitution of some or all uridine residues with a modified uridine residue.
30. The composition of any one of embodiments 1-29, wherein the RNA is modified by N1-methyl-pseudouridine substitution of some or all uridine residues.
31. The composition of one of embodiments 1-27, wherein the RNA is formulated in lipid nanoparticles comprising a cationic or cationically ionizable lipid, a sterol, a neutral lipid, and a lipid conjugate.
32. The composition of one of embodiments 1-28, wherein the RNA is formulated in lipid nanoparticles comprising a cationically ionizable lipid, a phospholipid, a cholesterol, and a polyethylene glycol (PEG)-lipid.
33. The composition of embodiment 31 or 32, wherein the cationic lipid or cationically ionizable is or comprises ((4-hydroxybutyl)azanediyl)bis(hexane-6,1-diyl)bis(2-hexyldecanoate), the sterol is or comprises a cholesterol, the neutral lipid is or comprises a phospholipid, and the lipid conjugate is or comprises a polyethylene glycol (PEG)-lipid.
34. The composition according to any one of embodiments 31-33, wherein the one or more lipid nanoparticles comprise:
   a. ((4-hydroxybutyl)azanediyl)bis(hexane-6,1-diyl)bis(2-hexyldecanoate);
   b. a cholesterol;
   c. distearoylphosphatidylcholine (DSPC); and
   d. 2-[(polyethylene glycol)-2000]—N,N-ditetradecylacetamide.
35. The composition of embodiment 31 or 32, wherein the cationically ionizable lipid has the following structure:

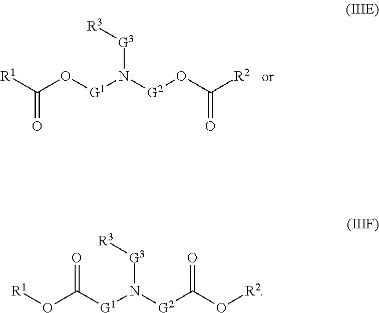

or a pharmaceutically acceptable salt, tautomer or stereoisomer thereof, wherein:
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl
36. The composition of embodiment 35, wherein R¹ or R², or both, has one of the following structures:

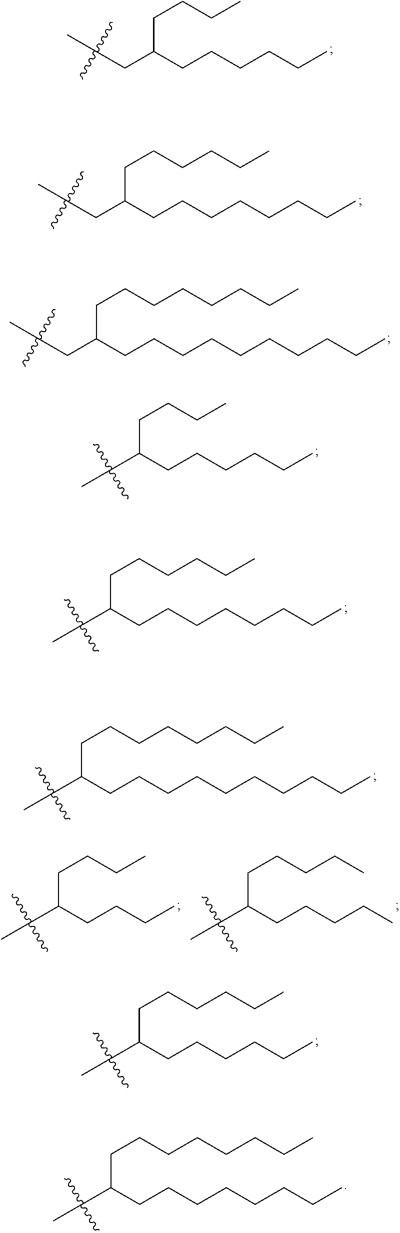

37. The composition of embodiment 35, wherein the cationically ionizable lipid has the following structure:

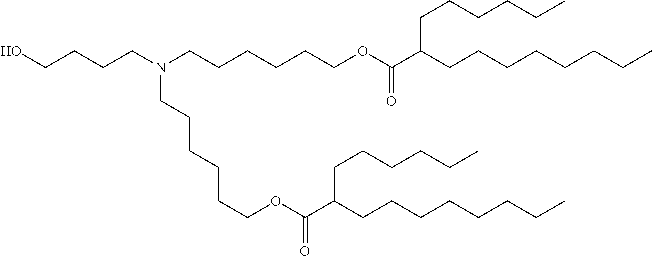 [SMILES: CCCCCCCCC(CCCCCC)C(=O)OCCCCCCN(CCCCO)CCCCCCOC(=O)C(CCCCCC)CCCCCCCC]

38. The composition of any one of embodiments 32-37, wherein the phospholipid is or comprises distearoylphosphatidylcholine (DSPC).
39. The pharmaceutical composition any one of embodiments 32-38, wherein the (PEG)-lipid is or comprises 2-[(polyethylene glycol)-2000]—N,N-ditetradecylacetamide.
40. The composition of any one of embodiments 32-39, wherein the phospholipid is present in a concentration ranging from 5 to 15 mol percent of the total lipids.
41. The composition of any one of embodiments 31-40, wherein the cationically ionizable lipid is present in a concentration ranging from 40 to 55 mol percent of the total lipids.
42. The composition of any one of embodiments 32-41, wherein the cholesterol is present in a concentration ranging from 30 to 50 mol percent of the total lipids.
43. The composition of any one of embodiments 32-41, wherein the (PEG)-lipid is present in a concentration ranging from 1 to 10 mol percent of the total lipids.
44. The composition of any one of embodiments 32-43, wherein the lipid nanoparticles comprise from 40 to 55 mol percent of a cationically ionizable lipid; from 5 to 15 mol percent of a phospholipid; from 30 to 50 mol percent of a cholesterol; and from 1 to 10 mol percent of a PEG-lipid.
45. The composition of embodiment 34, wherein ((4-hydroxybutyl)azanediyl)bis(hexane-6,1-diyl)bis(2-hexyldecanoate) is within a range of about 40 to about 55 mole percent, a cholesterol is within a range of about 30 to about 50 mole percent, distearoylphosphatidylcholine (DSPC) is within a range of about 5 to about 15 mole percent, and 2-[(polyethylene glycol)-2000]—N,N-ditetradecylacetamide is within a range of about 1 to about 10 mole percent.
46. The composition of any one of embodiments 1-45, further comprising at least one salt and/or a cryoprotectant, wherein the cryoprotectant is or comprises sucrose.
47. The composition of any one of embodiments 1-46, wherein the total RNA is present in an amount within a range of 1 ug to 100 ug per dose in the composition.
48. A pharmaceutical composition comprising a composition of any one of embodiments
1-47. 49. The pharmaceutical composition of embodiment 48, which is in a liquid formulation.
50. The pharmaceutical composition of embodiment 48, which is in a frozen formulation.
51. The pharmaceutical composition of embodiment 50, wherein the frozen formulation comprises PBS.
52. The pharmaceutical composition of any one of embodiments 48-51, wherein the composition is formulated for intramuscular administration.
53. The pharmaceutical composition of any one of embodiments 48-51, wherein the composition is formulated for intravenous administration.
54. The pharmaceutical composition of any one of embodiments 48-51, wherein the composition is formulated for subcutaneous administration.
55. A method comprising administering at least one dose of a pharmaceutical composition of any one of embodiments 48-54 to a subject.
56. The method of embodiment 55, wherein the subject is human.
57. The method of embodiment 55 or 56, wherein the subject is suffering from a VZV infection, e.g., varicella or herpes zoster.
58. The method of embodiment 55 or 56, wherein the subject intends to be present within a geographical region that has a high VZV prevalence within the next three months.
59. The method of embodiment 58, wherein a high VZV prevalence is greater than 10% of the population.
60. The method of any one of embodiments 55-57, wherein the subject has previously been treated for a VZV infection, e.g., varicella or herpes zoster with a different pharmaceutical composition.
61. The method of any one of embodiments 55-60, further comprising administering a second dose of the pharmaceutical composition to the patient.
62. The method of any one of embodiments 55-61, further comprising administering at least two doses of the pharmaceutical composition to the patient.
63. The method of any one of embodiments 55-62, further comprising administering at least three doses of the pharmaceutical composition to the patient.
64. The method of any one of embodiments 55-63, wherein the method is a method of inducing an anti-VZV immune response in the subject.
65. The method of embodiment 64, wherein the immune response in the subject comprises an adaptive immune response.
66. The method of embodiment 64 or 65, wherein the immune response in the subject comprises a T-cell response.
67. The method of embodiment 66, wherein the T-cell response is or comprises a CD4+ response.
68. The method of embodiment 66 or 67, wherein the T-cell response is or comprises a CD8+ response.
69. The method of any one of embodiments 64-68, wherein the immune system response comprises a B-cell response.
70. The method of any one of embodiments 64-69, wherein the immune system response comprises the production of antibodies directed against the one or more VZV antigens.
71. The pharmaceutical composition of any one of embodiments 48-54 for use in the treatment of a VZV infection, e.g., varicella or herpes zoster.
72. The pharmaceutical composition of any one of embodiments 48-54 for use in inducing an anti-VZV immune response in the subject.
73. Use of the pharmaceutical composition of any one of embodiments 48-54 in the treatment of a VZV infection, e.g., varicella or herpes zoster.
74. Use of the pharmaceutical composition of any one of embodiments 48-54 in inducing an anti-VZV immune response in the subject.
75. A polypeptide comprising one or more of VZV antigens.
76. The polypeptide of embodiment 75, wherein the one or more VZV antigens comprise one or more VZV antigens listed in Table 3A, Table 3B, Table 4A, and/or Tables 5A-5B herein or comprise one or more VZV antigens encoded by respective genes listed in Tables 1A-1I and/or Tables 2A-2B herein.
77. A polynucleotide encoding one or more of VZV antigens.
78. The polynucleotide of embodiment 77, wherein the one or more VZV antigens comprise one or more VZV antigens listed in Table 3A, Table 3B, Table 4A, and/or Tables 5A-5B herein or comprise one or more VZV antigens encoded by respective genes listed in Tables 1A-1I and/or Tables 2A-2B herein.
79. The polynucleotide of embodiment 78, wherein the polynucleotide is DNA or RNA.
80. A cell comprising a polypeptide of embodiment 75 or 76 and/or a polynucleotide of any one of embodiments 77-79.
81. A cell comprising a polynucleotide of any one of embodiments 77-79.
82. The cell of embodiment 81, wherein the cell expresses the one or more VZV antigens encoded by the polynucleotide.

### Exemplary CMV Related Embodiments

Exemplary embodiments as described below are also within the scope of the present disclosure:
1. A composition for delivery of one or more CMV antigens to a subject.
2. A composition comprising one or more RNA molecules that collectively encode one or more CMV antigens.
3. The composition of embodiment 1 or 2, wherein the one or more CMV antigens comprise one or more CMV antigens listed in Table 8A, Table 8B, Table 9A, and/or Tables 10A-10B herein or comprise one or more CMV antigens encoded by respective genes listed in Tables 6A-6F and/or Tables 7A-7B herein.
4. The composition of any one of embodiments 1-3, wherein the one or more RNA molecules encode one or more amino acid sequences found in Table 8A, Table 8B, Table 9A, Table 9B, Table 10A, and/or Table 10B herein.
5. The composition of any one of embodiments 1-4, wherein the one or more RNA molecules comprise one or more sequences found in Table 9A and/or Table 9B herein.
6. The composition of any one of embodiments 1-5, wherein at least one RNA molecule of the one or more RNA molecules encodes a single CMV antigen.
7. The composition of any one of embodiments 1-6, wherein at least one RNA molecule of the one or more RNA molecules encodes multiple CMV antigens.
8. The composition of embodiment 7, wherein the at least one RNA molecule has a sequence found in Table 9A and/or Table 9B herein.
9. The composition of any one of embodiments 1-5, 7 or 8, wherein all of the one or more RNA molecules encode multiple CMV antigens.
10. The composition of embodiment 9, wherein the one or more RNA molecules each have a sequence found in Table 9A and/or Table 9B herein.
11. The composition of embodiment 9 or 10, wherein the one or more RNA molecules each have a sequence found in Table 9A and/or Table 9B herein, and the one or more RNA molecules comprise RNA molecules having different sequences.
12. The composition of any one of embodiments 8-11, wherein the one or more RNA molecules encoding multiple CMV antigens further comprise a linker.
13. The composition of embodiment 12, wherein the linker has a sequence found in Table 22 herein.
14. The composition of any one of embodiments 1-13, wherein the one or more RNA molecules comprises a 5′ cap or 5′ cap analog.
15. The composition of embodiment 14, wherein the 5′ cap analog is or comprises Cap0, a Cap1 or a Cap2.
16. The composition of embodiment 14 or 15, wherein a 5′-cap analog is or comprises m₂^{7,3′-O}Gppp(m₁^{2′-O})ApG.
17. The composition of any one of embodiments 1-16, wherein the one or more RNA molecules comprises a sequence encoding a signal peptide.
18. The composition of embodiment 17, wherein the signal peptide is or comprises a sequence found in Table 20 herein.
19. The composition of embodiment 17, wherein the one or more RNA molecules comprise a sequence encoding a signal peptide found in Table 21 herein.
20. The composition of any one of embodiments 1-19, wherein the one or more RNA molecules comprise at least one non-coding regulatory element.
21. The composition of any one of embodiments 1-20, wherein the one or more RNA molecules comprises a poly-adenine tail.
22. The composition of embodiment 21, wherein the poly-adenine tail is or comprises a modified adenine sequence.
23. The composition of embodiment 21 or 22, wherein the poly-adenine tail comprises at least 100 A nucleotides.
24. The composition of any one of embodiments 21-23, wherein the poly-adenine tail is an interrupted sequence of A nucleotides.
25. The composition according to embodiment 24, wherein the poly-adenine tail comprises 30 adenine nucleotides followed by 70 adenine nucleotides, wherein the 30 adenine nucleotides and 70 adenine nucleotides are separated by a linker sequence.
26. The composition of any one of embodiments 1-25, wherein the one or more RNA molecules comprises at least one 5′ untranslated region (UTR) and/or at least one 3′ UTR.
27. The composition according to embodiment 26, wherein the at least one 5′-UTR is or comprises a modified human alpha-globin 5′-UTR.
28. The composition according to embodiment 26 or 27, wherein the at least one 3′-UTR is or comprises a first sequence from the “amino terminal enhancer of split” (AES) messenger RNA and a second sequence from the mitochondrial encoded 12S ribosomal RNA.
29. The composition of any one of embodiments 1-28, wherein the RNA is a modified RNA, which is modified by substitution of some or all uridine residues with a modified uridine residue.
30. The composition of any one of embodiments 1-29, wherein the RNA is modified by N1-methyl-pseudouridine substitution of some or all uridine residues.
31. The composition of one of embodiments 1-27, wherein the RNA is formulated in lipid nanoparticles comprising a cationic or cationically ionizable lipid, a sterol, a neutral lipid, and a lipid conjugate.
32. The composition of one of embodiments 1-28, wherein the RNA is formulated in lipid nanoparticles comprising a cationically ionizable lipid, a phospholipid, a cholesterol, and a polyethylene glycol (PEG)-lipid.
33. The composition of embodiment 31 or 32, wherein the cationic lipid or cationically ionizable is or comprises ((4-hydroxybutyl)azanediyl)bis(hexane-6,1-diyl)bis(2-hexyldecanoate), the sterol is or comprises a cholesterol, the neutral lipid is or comprises a phospholipid, and the lipid conjugate is or comprises a polyethylene glycol (PEG)-lipid.
34. The composition according to any one of embodiments 31-33, wherein the one or more lipid nanoparticles comprise:
   a. ((4-hydroxybutyl)azanediyl)bis(hexane-6,1-diyl)bis(2-hexyldecanoate);
   b. a cholesterol;
   c. distearoylphosphatidylcholine (DSPC); and
   d. 2-[(polyethylene glycol)-2000]—N,N-ditetradecylacetamide.
35. The composition of embodiment 31 or 32, wherein the cationically ionizable lipid has the following structure:

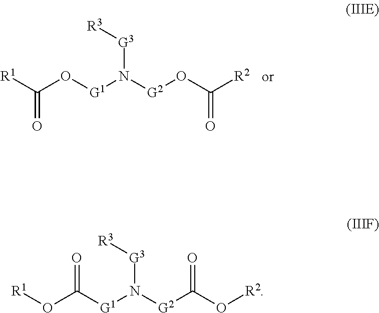

or a pharmaceutically acceptable salt, tautomer or stereoisomer thereof, wherein:
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl
36. The composition of embodiment 35, wherein R¹ or R², or both, has one of the following structures:

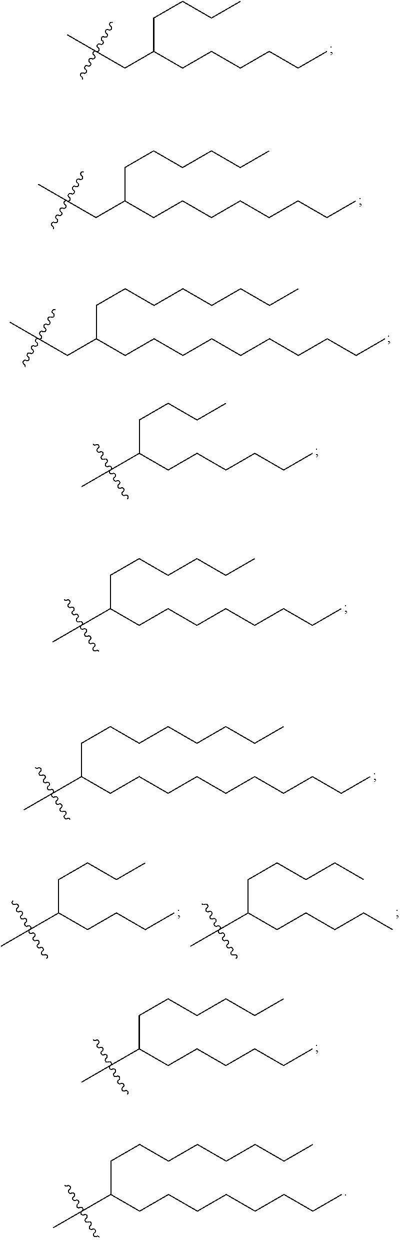

37. The composition of embodiment 35, wherein the cationically ionizable lipid has the following structure:

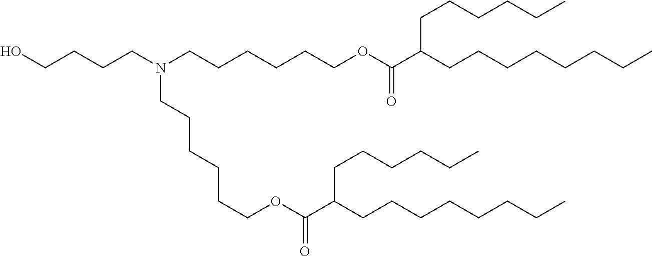 [SMILES: CCCCCCCCC(CCCCCC)C(=O)OCCCCCCN(CCCCO)CCCCCCOC(=O)C(CCCCCC)CCCCCCCC]

38. The composition of any one of embodiments 32-37, wherein the phospholipid is or comprises distearoylphosphatidylcholine (DSPC).
39. The pharmaceutical composition any one of embodiments 32-38, wherein the (PEG)-lipid is or comprises 2-[(polyethylene glycol)-2000]—N,N-ditetradecylacetamide.
40. The composition of any one of embodiments 32-39, wherein the phospholipid is present in a concentration ranging from 5 to 15 mol percent of the total lipids.
41. The composition of any one of embodiments 31-40, wherein the cationically ionizable lipid is present in a concentration ranging from 40 to 55 mol percent of the total lipids.
42. The composition of any one of embodiments 32-41, wherein the cholesterol is present in a concentration ranging from 30 to 50 mol percent of the total lipids.
43. The composition of any one of embodiments 32-41, wherein the (PEG)-lipid is present in a concentration ranging from 1 to 10 mol percent of the total lipids.
44. The composition of any one of embodiments 32-43, wherein the lipid nanoparticles comprise from 40 to 55 mol percent of a cationically ionizable lipid; from 5 to 15 mol percent of a phospholipid; from 30 to 50 mol percent of a cholesterol; and from 1 to 10 mol percent of a PEG-lipid.
45. The composition of embodiment 34, wherein ((4-hydroxybutyl)azanediyl)bis(hexane-6,1-diyl)bis(2-hexyldecanoate) is within a range of about 40 to about 55 mole percent, a cholesterol is within a range of about 30 to about 50 mole percent, distearoylphosphatidylcholine (DSPC) is within a range of about 5 to about 15 mole percent, and 2-[(polyethylene glycol)-2000]—N,N-ditetradecylacetamide is within a range of about 1 to about 10 mole percent.
46. The composition of any one of embodiments 1-45, further comprising at least one salt and/or a cryoprotectant, wherein the cryoprotectant is or comprises sucrose.
47. The composition of any one of embodiments 1-46, wherein the total RNA is present in an amount within a range of 1 ug to 100 ug per dose in the composition.
48. A pharmaceutical composition comprising a composition of any one of embodiments 1-47. 49. The pharmaceutical composition of embodiment 48, which is in a liquid formulation.
50. The pharmaceutical composition of embodiment 48, which is in a frozen formulation.
51. The pharmaceutical composition of embodiment 50, wherein the frozen formulation comprises PBS.
52. The pharmaceutical composition of any one of embodiments 48-51, wherein the composition is formulated for intramuscular administration.
53. The pharmaceutical composition of any one of embodiments 48-51, wherein the composition is formulated for intravenous administration.
54. The pharmaceutical composition of any one of embodiments 48-51, wherein the composition is formulated for subcutaneous administration.
55. A method comprising administering at least one dose of a pharmaceutical composition of any one of embodiments 48-54 to a subject.
56. The method of embodiment 55, wherein the subject is human.
57. The method of embodiment 55 or 56, wherein the subject is suffering from a CMV infection, e.g., varicella or herpes zoster.
58. The method of embodiment 55 or 56, wherein the subject intends to be present within a geographical region that has a high CMV prevalence within the next three months.
59. The method of embodiment 58, wherein a high CMV prevalence is greater than 10% of the population.
60. The method of any one of embodiments 55-57, wherein the subject has previously been treated for a CMV infection, e.g., varicella or herpes zoster with a different pharmaceutical composition.
61. The method of any one of embodiments 55-60, further comprising administering a second dose of the pharmaceutical composition to the patient.
62. The method of any one of embodiments 55-61, further comprising administering at least two doses of the pharmaceutical composition to the patient.
63. The method of any one of embodiments 55-62, further comprising administering at least three doses of the pharmaceutical composition to the patient.
64. The method of any one of embodiments 55-63, wherein the method is a method of inducing an anti-CMV immune response in the subject.
65. The method of embodiment 64, wherein the immune response in the subject comprises an adaptive immune response.
66. The method of embodiment 64 or 65, wherein the immune response in the subject comprises a T-cell response.
67. The method of embodiment 66, wherein the T-cell response is or comprises a CD4+ response.
68. The method of embodiment 66 or 67, wherein the T-cell response is or comprises a CD8+ response.
69. The method of any one of embodiments 64-68, wherein the immune system response comprises a B-cell response.
70. The method of any one of embodiments 64-69, wherein the immune system response comprises the production of antibodies directed against the one or more CMV antigens.
71. The pharmaceutical composition of any one of embodiments 48-54 for use in the treatment of a CMV infection, e.g., varicella or herpes zoster.
72. The pharmaceutical composition of any one of embodiments 48-54 for use in inducing an anti-CMV immune response in the subject.
73. Use of the pharmaceutical composition of any one of embodiments 48-54 in the treatment of a CMV infection, e.g., varicella or herpes zoster.
74. Use of the pharmaceutical composition of any one of embodiments 48-54 in inducing an anti-CMV immune response in the subject.
75. A polypeptide comprising one or more of CMV antigens.
76. The polypeptide of embodiment 75, wherein the one or more CMV antigens comprise one or more CMV antigens listed in Table 8A, Table 8B, Table 9A, and/or Tables 10A-10B herein or comprise one or more CMV antigens encoded by respective genes listed in Tables 6A-6F and/or Tables 7A-7B herein.
77. A polynucleotide encoding one or more of CMV antigens.
78. The polynucleotide of embodiment 77, wherein the one or more CMV antigens comprise one or more CMV antigens listed in Table 8A, Table 8B, Table 9A, and/or Tables 10A-10B herein or comprise one or more CMV antigens encoded by respective genes listed in Tables 6A-6F and/or Tables 7A-7B herein.
79. The polynucleotide of embodiment 78, wherein the polynucleotide is DNA or RNA.
80. A cell comprising a polypeptide of embodiment 75 or 76 and/or a polynucleotide of any one of embodiments 77-79.
81. A cell comprising a polynucleotide of any one of embodiments 77-79.
82. The cell of embodiment 81, wherein the cell expresses the one or more CMV antigens encoded by the polynucleotide.

### Exemplary Norovirus Related Embodiments

Exemplary embodiments as described below are also within the scope of the present disclosure:
1. A composition for delivery of one or more norovirus antigens to a subject.
2. A composition comprising one or more RNA molecules that collectively encode one or more norovirus antigens.
3. The composition of embodiment 1 or 2, wherein the one or more norovirus antigens comprise one or more norovirus antigens listed in Tables 14A-14N, Table 15A, and/or Tables 16A-16P herein or comprise one or more norovirus antigens encoded by respective genes listed in Tables 12A-12D and/or Tables 13A-13B herein.
4. The composition of any one of embodiments 1-3, wherein the one or more RNA molecules encode one or more amino acid sequences found in Tables 14A-14N, Table 15A-15B, and/or Tables 16A-16P herein.
5. The composition of any one of embodiments 1-4, wherein the one or more RNA molecules comprise one or more sequences found in Tables 14A-14N, and/or Table 15A-15B herein.
6. The composition of any one of embodiments 1-5, wherein at least one RNA molecule of the one or more RNA molecules encodes a single norovirus antigen.
7. The composition of any one of embodiments 1-6, wherein at least one RNA molecule of the one or more RNA molecules encodes multiple norovirus antigens.
8. The composition of embodiment 7, wherein the at least one RNA molecule has a sequence found in Tables 14A-14N, and/or Table 15A-15B herein.
9. The composition of any one of embodiments 1-5, 7 or 8, wherein all of the one or more RNA molecules encode multiple norovirus antigens.
10. The composition of embodiment 9, wherein the one or more RNA molecules each have a sequence found in Tables 14A-14N, and/or Table 15A-15B herein.
11. The composition of embodiment 9 or 10, wherein the one or more RNA molecules each have a sequence found in Tables 14A-14N, and/or Table 15A-15B herein, and the one or more RNA molecules comprise RNA molecules having different sequences.
12. The composition of any one of embodiments 8-11, wherein the one or more RNA molecules encoding multiple norovirus antigens further comprise a linker.
13. The composition of embodiment 12, wherein the linker has a sequence found in Table 22 herein.
14. The composition of any one of embodiments 1-13, wherein the one or more RNA molecules comprises a 5′ cap or 5′ cap analog.
15. The composition of embodiment 14, wherein the 5′ cap analog is or comprises Cap0, a Cap1 or a Cap2.
16. The composition of embodiment 14 or 15, wherein a 5′-cap analog is or comprises m₂^{7,3′-O}Gppp(m₁^{2′-O})ApG.
17. The composition of any one of embodiments 1-16, wherein the one or more RNA molecules comprises a sequence encoding a signal peptide.
18. The composition of embodiment 17, wherein the signal peptide is or comprises a sequence found in Table 20 herein.
19. The composition of embodiment 17, wherein the one or more RNA molecules comprise a sequence encoding a signal peptide found in Table 21 herein.
20. The composition of any one of embodiments 1-19, wherein the one or more RNA molecules comprise at least one non-coding regulatory element.
21. The composition of any one of embodiments 1-20, wherein the one or more RNA molecules comprises a poly-adenine tail.
22. The composition of embodiment 21, wherein the poly-adenine tail is or comprises a modified adenine sequence.
23. The composition of embodiment 21 or 22, wherein the poly-adenine tail comprises at least 100 A nucleotides.
24. The composition of any one of embodiments 21-23, wherein the poly-adenine tail is an interrupted sequence of A nucleotides.
25. The composition according to embodiment 24, wherein the poly-adenine tail comprises 30 adenine nucleotides followed by 70 adenine nucleotides, wherein the 30 adenine nucleotides and 70 adenine nucleotides are separated by a linker sequence.
26. The composition of any one of embodiments 1-25, wherein the one or more RNA molecules comprises at least one 5′ untranslated region (UTR) and/or at least one 3′ UTR.
27. The composition according to embodiment 26, wherein the at least one 5′-UTR is or comprises a modified human alpha-globin 5′-UTR.
28. The composition according to embodiment 26 or 27, wherein the at least one 3′-UTR is or comprises a first sequence from the “amino terminal enhancer of split” (AES) messenger RNA and a second sequence from the mitochondrial encoded 12S ribosomal RNA.
29. The composition of any one of embodiments 1-28, wherein the RNA is a modified RNA, which is modified by substitution of some or all uridine residues with a modified uridine residue.
30. The composition of any one of embodiments 1-29, wherein the RNA is modified by N1-methyl-pseudouridine substitution of some or all uridine residues.
31. The composition of one of embodiments 1-27, wherein the RNA is formulated in lipid nanoparticles comprising a cationic or cationically ionizable lipid, a sterol, a neutral lipid, and a lipid conjugate.
32. The composition of one of embodiments 1-28, wherein the RNA is formulated in lipid nanoparticles comprising a cationically ionizable lipid, a phospholipid, a cholesterol, and a polyethylene glycol (PEG)-lipid.
33. The composition of embodiment 31 or 32, wherein the cationic lipid or cationically ionizable is or comprises ((4-hydroxybutyl)azanediyl)bis(hexane-6,1-diyl)bis(2-hexyldecanoate), the sterol is or comprises a cholesterol, the neutral lipid is or comprises a phospholipid, and the lipid conjugate is or comprises a polyethylene glycol (PEG)-lipid.
34. The composition according to any one of embodiments 31-33, wherein the one or more lipid nanoparticles comprise:
   a. ((4-hydroxybutyl)azanediyl)bis(hexane-6,1-diyl)bis(2-hexyldecanoate);
   b. a cholesterol;
   c. distearoylphosphatidylcholine (DSPC); and
   d. 2-[(polyethylene glycol)-2000]—N,N-ditetradecylacetamide.
35. The composition of embodiment 31 or 32, wherein the cationically ionizable lipid has the following structure:

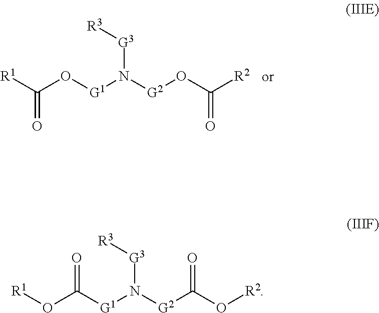

or a pharmaceutically acceptable salt, tautomer or stereoisomer thereof, wherein:
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl
36. The composition of embodiment 35, wherein R¹ or R², or both, has one of the following structures:

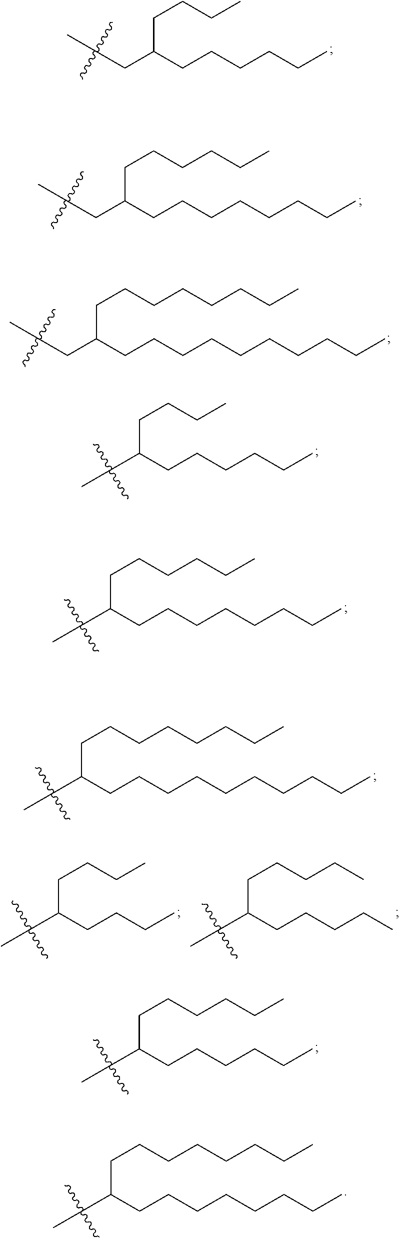

37. The composition of embodiment 35, wherein the cationically ionizable lipid has the following structure:

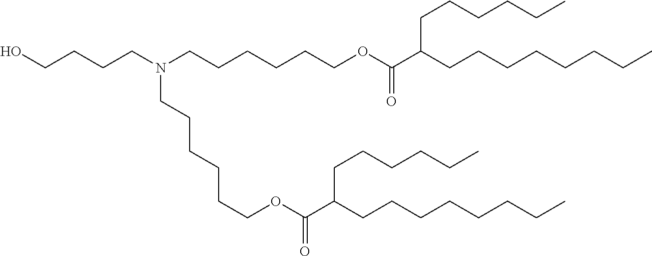 [SMILES: CCCCCCCCC(CCCCCC)C(=O)OCCCCCCN(CCCCO)CCCCCCOC(=O)C(CCCCCC)CCCCCCCC]

38. The composition of any one of embodiments 32-37, wherein the phospholipid is or comprises distearoylphosphatidylcholine (DSPC).
39. The pharmaceutical composition any one of embodiments 32-38, wherein the (PEG)-lipid is or comprises 2-[(polyethylene glycol)-2000]—N,N-ditetradecylacetamide.
40. The composition of any one of embodiments 32-39, wherein the phospholipid is present in a concentration ranging from 5 to 15 mol percent of the total lipids.
41. The composition of any one of embodiments 31-40, wherein the cationically ionizable lipid is present in a concentration ranging from 40 to 55 mol percent of the total lipids.
42. The composition of any one of embodiments 32-41, wherein the cholesterol is present in a concentration ranging from 30 to 50 mol percent of the total lipids.
43. The composition of any one of embodiments 32-41, wherein the (PEG)-lipid is present in a concentration ranging from 1 to 10 mol percent of the total lipids.
44. The composition of any one of embodiments 32-43, wherein the lipid nanoparticles comprise from 40 to 55 mol percent of a cationically ionizable lipid; from 5 to 15 mol percent of a phospholipid; from 30 to 50 mol percent of a cholesterol; and from 1 to 10 mol percent of a PEG-lipid.
45. The composition of embodiment 34, wherein ((4-hydroxybutyl)azanediyl)bis(hexane-6,1-diyl)bis(2-hexyldecanoate) is within a range of about 40 to about 55 mole percent, a cholesterol is within a range of about 30 to about 50 mole percent, distearoylphosphatidylcholine (DSPC) is within a range of about 5 to about 15 mole percent, and 2-[(polyethylene glycol)-2000]—N,N-ditetradecylacetamide is within a range of about 1 to about 10 mole percent.
46. The composition of any one of embodiments 1-45, further comprising at least one salt and/or a cryoprotectant, wherein the cryoprotectant is or comprises sucrose.
47. The composition of any one of embodiments 1-46, wherein the total RNA is present in an amount within a range of 1 ug to 100 ug per dose in the composition.
48. A pharmaceutical composition comprising a composition of any one of embodiments 1-47.
49. The pharmaceutical composition of embodiment 48, which is in a liquid formulation.
50. The pharmaceutical composition of embodiment 48, which is in a frozen formulation.
51. The pharmaceutical composition of embodiment 50, wherein the frozen formulation comprises PBS.
52. The pharmaceutical composition of any one of embodiments 48-51, wherein the composition is formulated for intramuscular administration.
53. The pharmaceutical composition of any one of embodiments 48-51, wherein the composition is formulated for intravenous administration.
54. The pharmaceutical composition of any one of embodiments 48-51, wherein the composition is formulated for subcutaneous administration.
55. A method comprising administering at least one dose of a pharmaceutical composition of any one of embodiments 48-54 to a subject.
56. The method of embodiment 55, wherein the subject is human.
57. The method of embodiment 55 or 56, wherein the subject is suffering from a norovirus infection, e.g., by a specific genogroup and/or genotype (e.g., norovirus GII.4).
58. The method of embodiment 55 or 56, wherein the subject intends to be present within a geographical region that has a high norovirus prevalence within the next three months.
59. The method of embodiment 58, wherein a high norovirus prevalence is greater than 10% of the population.
60. The method of any one of embodiments 55-57, wherein the subject has previously been treated for a norovirus infection, e.g., by a specific genogroup and/or genotype (e.g., norovirus GII.4), with a different pharmaceutical composition.
61. The method of any one of embodiments 55-60, further comprising administering a second dose of the pharmaceutical composition to the patient.
62. The method of any one of embodiments 55-61, further comprising administering at least two doses of the pharmaceutical composition to the patient.
63. The method of any one of embodiments 55-62, further comprising administering at least three doses of the pharmaceutical composition to the patient.
64. The method of any one of embodiments 55-63, wherein the method is a method of inducing an anti-norovirus immune response in the subject.
65. The method of embodiment 64, wherein the immune response in the subject comprises an adaptive immune response.
66. The method of embodiment 64 or 65, wherein the immune response in the subject comprises a T-cell response.
67. The method of embodiment 66, wherein the T-cell response is or comprises a CD4+ response.
68. The method of embodiment 66 or 67, wherein the T-cell response is or comprises a CD8+ response.
69. The method of any one of embodiments 64-68, wherein the immune system response comprises a B-cell response.
70. The method of any one of embodiments 64-69, wherein the immune system response comprises the production of antibodies directed against the one or more norovirus antigens.
71. The pharmaceutical composition of any one of embodiments 48-54 for use in the treatment of a norovirus infection, e.g., by a specific genogroup and/or genotype (e.g., norovirus GII.4).
72. The pharmaceutical composition of any one of embodiments 48-54 for use in inducing an anti-norovirus immune response in the subject.
73. Use of the pharmaceutical composition of any one of embodiments 48-54 in the treatment of a norovirus infection, e.g., by a specific genogroup and/or genotype (e.g., norovirus GII.4).
74. Use of the pharmaceutical composition of any one of embodiments 48-54 in inducing an anti-norovirus immune response in the subject.
75. A polypeptide comprising one or more of norovirus antigens.
76. The polypeptide of embodiment 75, wherein the one or more norovirus antigens comprise one or more norovirus antigens listed in Tables 14A-14N, Table 15A, and/or Tables 16A-16P herein or comprise one or more norovirus antigens encoded by respective genes listed in Tables 12A-12D and/or Tables 13A-13B herein.
77. A polynucleotide encoding one or more of norovirus antigens.
78. The polynucleotide of embodiment 77, wherein the one or more norovirus antigens comprise one or more norovirus antigens listed in Tables 14A-14N, Table 15A, and/or Tables 16A-16P herein or comprise one or more norovirus antigens encoded by respective genes listed in Tables 12A-12D and/or Tables 13A-13B herein.
79. The polynucleotide of embodiment 78, wherein the polynucleotide is DNA or RNA.
80. A cell comprising a polypeptide of embodiment 75 or 76 and/or a polynucleotide of any one of embodiments 77-79.
81. A cell comprising a polynucleotide of any one of embodiments 77-79.
82. The cell of embodiment 81, wherein the cell expresses the one or more norovirus antigens encoded by the polynucleotide.

### Tables:

This patent application also contains tables below. Information in the tables may be employed in the practice of the invention. The tables that follow are:
1. Tables 1A-1I;
2. Tables 2A-2B;
3. Tables 3A-3B;
4. Tables 4A-4B;
5. Tables 5A-5B;
6. Tables 6A-6F;
7. Tables 7A-7B;
8. Tables 8A-8B;
9. Tables 9A-9B;
10. Tables 10A-10B;
11. Table 11;
12. Tables 12A-12D;
13. Tables 13A-13B;
14. Tables 14A-14N;
15. Tables 15A-15B;
16. Tables 16A-16P; and
17. Table 24.

### SEQUENCE LISTING

As referenced in the Sequence Listing section above, this patent application contains a Sequence Listing, which has been submitted via DVD-R. The Sequence Listing includes sequences that may be employed in the practice of the invention. Said DVD-R, recorded on Oct. 14, 2022, is labeled 2013237-0466-SL.zip and contains a 664,937,000 byte file.

### EQUIVALENTS

Those skilled in the art will recognize, or be able to ascertain using no more than routine experimentation, many equivalents to the specific embodiments of technologies described herein. The scope of the present disclosure is not intended to be limited to the above Description, but rather is as set forth in the following claims:

Lengthy table referenced here
US20240366754A1-20241107-T00001
Please refer to the end of the specification for access instructions.

Lengthy table referenced here
US20240366754A1-20241107-T00002
Please refer to the end of the specification for access instructions.

Lengthy table referenced here
US20240366754A1-20241107-T00003
Please refer to the end of the specification for access instructions.

Lengthy table referenced here
US20240366754A1-20241107-T00004
Please refer to the end of the specification for access instructions.

Lengthy table referenced here
US20240366754A1-20241107-T00005
Please refer to the end of the specification for access instructions.

Lengthy table referenced here
US20240366754A1-20241107-T00006
Please refer to the end of the specification for access instructions.

Lengthy table referenced here
US20240366754A1-20241107-T00007
Please refer to the end of the specification for access instructions.

Lengthy table referenced here
US20240366754A1-20241107-T00008
Please refer to the end of the specification for access instructions.

Lengthy table referenced here
US20240366754A1-20241107-T00009
Please refer to the end of the specification for access instructions.

Lengthy table referenced here
US20240366754A1-20241107-T00010
Please refer to the end of the specification for access instructions.

Lengthy table referenced here
US20240366754A1-20241107-T00011
Please refer to the end of the specification for access instructions.

Lengthy table referenced here
US20240366754A1-20241107-T00012
Please refer to the end of the specification for access instructions.

Lengthy table referenced here
US20240366754A1-20241107-T00013
Please refer to the end of the specification for access instructions.

Lengthy table referenced here
US20240366754A1-20241107-T00014
Please refer to the end of the specification for access instructions.

Lengthy table referenced here
US20240366754A1-20241107-T00015
Please refer to the end of the specification for access instructions.

Lengthy table referenced here
US20240366754A1-20241107-T00016
Please refer to the end of the specification for access instructions.

Lengthy table referenced here
US20240366754A1-20241107-T00017
Please refer to the end of the specification for access instructions.

Lengthy table referenced here
US20240366754A1-20241107-T00018
Please refer to the end of the specification for access instructions.

Lengthy table referenced here
US20240366754A1-20241107-T00019
Please refer to the end of the specification for access instructions.

Lengthy table referenced here
US20240366754A1-20241107-T00020
Please refer to the end of the specification for access instructions.

Lengthy table referenced here
US20240366754A1-20241107-T00021
Please refer to the end of the specification for access instructions.

Lengthy table referenced here
US20240366754A1-20241107-T00022
Please refer to the end of the specification for access instructions.

Lengthy table referenced here
US20240366754A1-20241107-T00023
Please refer to the end of the specification for access instructions.

Lengthy table referenced here
US20240366754A1-20241107-T00024
Please refer to the end of the specification for access instructions.

Lengthy table referenced here
US20240366754A1-20241107-T00025
Please refer to the end of the specification for access instructions.

Lengthy table referenced here
US20240366754A1-20241107-T00026
Please refer to the end of the specification for access instructions.

Lengthy table referenced here
US20240366754A1-20241107-T00027
Please refer to the end of the specification for access instructions.

Lengthy table referenced here
US20240366754A1-20241107-T00028
Please refer to the end of the specification for access instructions.

Lengthy table referenced here
US20240366754A1-20241107-T00029
Please refer to the end of the specification for access instructions.

Lengthy table referenced here
US20240366754A1-20241107-T00030
Please refer to the end of the specification for access instructions.

Lengthy table referenced here
US20240366754A1-20241107-T00031
Please refer to the end of the specification for access instructions.

Lengthy table referenced here
US20240366754A1-20241107-T00032
Please refer to the end of the specification for access instructions.

Lengthy table referenced here
US20240366754A1-20241107-T00033
Please refer to the end of the specification for access instructions.

Lengthy table referenced here
US20240366754A1-20241107-T00034
Please refer to the end of the specification for access instructions.

Lengthy table referenced here
US20240366754A1-20241107-T00035
Please refer to the end of the specification for access instructions.

Lengthy table referenced here
US20240366754A1-20241107-T00036
Please refer to the end of the specification for access instructions.

Lengthy table referenced here
US20240366754A1-20241107-T00037
Please refer to the end of the specification for access instructions.

Lengthy table referenced here
US20240366754A1-20241107-T00038
Please refer to the end of the specification for access instructions.

Lengthy table referenced here
US20240366754A1-20241107-T00039
Please refer to the end of the specification for access instructions.

Lengthy table referenced here
US20240366754A1-20241107-T00040
Please refer to the end of the specification for access instructions.

Lengthy table referenced here
US20240366754A1-20241107-T00041
Please refer to the end of the specification for access instructions.

Lengthy table referenced here
US20240366754A1-20241107-T00042
Please refer to the end of the specification for access instructions.

Lengthy table referenced here
US20240366754A1-20241107-T00043
Please refer to the end of the specification for access instructions.

Lengthy table referenced here
US20240366754A1-20241107-T00044
Please refer to the end of the specification for access instructions.

Lengthy table referenced here
US20240366754A1-20241107-T00045
Please refer to the end of the specification for access instructions.

Lengthy table referenced here
US20240366754A1-20241107-T00046
Please refer to the end of the specification for access instructions.

Lengthy table referenced here
US20240366754A1-20241107-T00047
Please refer to the end of the specification for access instructions.

Lengthy table referenced here
US20240366754A1-20241107-T00048
Please refer to the end of the specification for access instructions.

Lengthy table referenced here
US20240366754A1-20241107-T00049
Please refer to the end of the specification for access instructions.

Lengthy table referenced here
US20240366754A1-20241107-T00050
Please refer to the end of the specification for access instructions.

Lengthy table referenced here
US20240366754A1-20241107-T00051
Please refer to the end of the specification for access instructions.

Lengthy table referenced here
US20240366754A1-20241107-T00052
Please refer to the end of the specification for access instructions.

Lengthy table referenced here
US20240366754A1-20241107-T00053
Please refer to the end of the specification for access instructions.

Lengthy table referenced here
US20240366754A1-20241107-T00054
Please refer to the end of the specification for access instructions.

Lengthy table referenced here
US20240366754A1-20241107-T00055
Please refer to the end of the specification for access instructions.

Lengthy table referenced here
US20240366754A1-20241107-T00056
Please refer to the end of the specification for access instructions.

Lengthy table referenced here
US20240366754A1-20241107-T00057
Please refer to the end of the specification for access instructions.

Lengthy table referenced here
US20240366754A1-20241107-T00058
Please refer to the end of the specification for access instructions.

Lengthy table referenced here
US20240366754A1-20241107-T00059
Please refer to the end of the specification for access instructions.

Lengthy table referenced here
US20240366754A1-20241107-T00060
Please refer to the end of the specification for access instructions.

Lengthy table referenced here
US20240366754A1-20241107-T00061
Please refer to the end of the specification for access instructions.

Lengthy table referenced here
US20240366754A1-20241107-T00062
Please refer to the end of the specification for access instructions.

Lengthy table referenced here
US20240366754A1-20241107-T00063
Please refer to the end of the specification for access instructions.

Lengthy table referenced here
US20240366754A1-20241107-T00064
Please refer to the end of the specification for access instructions.

Lengthy table referenced here
US20240366754A1-20241107-T00065
Please refer to the end of the specification for access instructions.

Lengthy table referenced here
US20240366754A1-20241107-T00066
Please refer to the end of the specification for access instructions.

Lengthy table referenced here
US20240366754A1-20241107-T00067
Please refer to the end of the specification for access instructions.

Lengthy table referenced here
US20240366754A1-20241107-T00068
Please refer to the end of the specification for access instructions.

Lengthy table referenced here
US20240366754A1-20241107-T00069
Please refer to the end of the specification for access instructions.

Lengthy table referenced here
US20240366754A1-20241107-T00070
Please refer to the end of the specification for access instructions.

Lengthy table referenced here
US20240366754A1-20241107-T00071
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20240366754A1). An electronic copy of the table will also be available from the USPTO upon request and payment of the fee set forth in 37 CFR 1.19(b)(3).

We claim:

1. A polyribonucleotide encoding a polypeptide, wherein the polypeptide comprises one or more antigens from a latent virus, wherein one or more antigens comprise at least one latent phase antigen, wherein the at least one latent phase antigen is obtained from a polypeptide expressed during the latent phase or the reactivation phase the latent virus lifecycle.

2. The polyribonucleotide of claim 1, wherein the one or more antigens comprise one or more T-cell antigens.

3. The polyribonucleotide of claim 1 or 2, wherein the at least one latent phase antigen comprises one or more T-cell antigens.

4. A polyribonucleotide encoding a polypeptide, wherein the polypeptide comprises one or more T-cell antigens that are expressed during the latent or the reactivation phase of a latent virus lifecycle.

5. The polyribonucleotide of any one of claims 2-4, wherein the one or more T-cell antigens comprises at least 2 and at most 30 T-cell antigens that are associated with a latent virus infection.

6. The polyribonucleotide of any one of claims 1-5, wherein the polypeptide comprises at least 25 amino acids and at most 2000 amino acids.

7. The polyribonucleotide of any one of claims 1-6, wherein the polypeptide comprises at least 25 amino acids and at most 1500 amino acids.

8. The polyribonucleotide of any one of claims 2-7, wherein the one or more T-cell antigens are or comprise CD4+ T-cell antigens.

9. The polyribonucleotide of any one of claims 2-8, wherein the one or more T-cell antigens are or comprise CD8+ T-cell antigens.

10. The polyribonucleotide of any one of claims 2-9, wherein the one or more T-cell antigens each comprise at least 21 amino acids.

11. The polyribonucleotide of any one of claims 2-10, wherein the one or more T-cell antigens are from a latent virus that is capable of infecting a human.

12. The polyribonucleotide of claim 11, wherein the latent virus is a virus of the Herpesviridae, Papillomaviridae, Parvoviridae, or Adenoviridae family.

13. The polyribonucleotide of claim 11, wherein the latent virus is HSV-1, HSV-2, VZV, Human Immunodeficiency Virus (HIV), Epstein-Barr Virus (EBV), CMV, HHV-6, HHV-7, KSHV (HHV-8), JC virus (JCV), BK virus (BKV), parvovirus, or adenovirus.

14. The polyribonucleotide of claim 11, wherein the latent virus is Varicella-Zoster virus.

15. The polyribonucleotide of claim 14, wherein the one or more T-cell antigens comprise two or more T-cell antigens each independently encoded by a gene listed in Tables 1A-1I and/or Tables 2A-2B herein.

16. The polyribonucleotide of claim 14, wherein the one or more T-cell antigens comprise two or more T-cell antigens each independently selected from Table 3A, Table 3B, Table 4A, and/or Tables 5A-5B herein.

17. The polyribonucleotide of claim 14, wherein the one or more T-cell antigens comprise two or more CD8+ T cell antigens each independently encoded by: ORF10, ORF18, ORF29, ORF31, ORF34, ORF53, ORF62, ORF67, or combinations thereof.

18. The polyribonucleotide of claim 14, wherein the one or more T-cell antigens comprise two or more CD8+ T cell antigens each independently encoded by: ORF10, ORF18, ORF29, ORF31, ORF34, ORF53, ORF67, or combinations thereof.

19. The polyribonucleotide of claim 14, wherein the one or more T cell antigens comprises two or more CD4+ T cell antigens each independently encoded by: ORF9, ORF10, ORF12, ORF18, ORF19, ORF23, ORF24, ORF28, ORF31, ORF36, ORF37, ORF38, ORF40, ORF44, ORF59, ORF62, ORF63, ORF67, ORF68, or combinations thereof.

20. The polyribonucleotide of claim 14, wherein the one or more T cell antigens comprises two or more CD4+ T cell antigens each independently encoded by: ORF10, ORF18, ORF23, ORF28, ORF36, ORF37, ORF38, ORF40, ORF44, or combinations thereof.

21. The polyribonucleotide of claim 11, wherein the latent virus is CMV.

22. The polyribonucleotide of claim 21, wherein the one or more T-cell antigens comprise two or more T-cell antigens each independently encoded by a gene listed in Tables 6A-6F and/or Tables 7A-7B herein.

23. The polyribonucleotide of claim 21, wherein the one or more T-cell antigens comprise two or more T-cell antigens each independently selected from Table 8A, Table 8B, Table 9A, and/or Tables 10A-10B herein.

24. The polyribonucleotide of claim 21, wherein the one or more T-cell antigens comprise two or more CD8+ T cell antigens each independently encoded by: TRS1, UL32, UL36, UL44, UL55, UL57, UL75, UL83, UL84, UL86, UL98, UL122, UL123, or combinations thereof.

25. The polyribonucleotide of claim 21, wherein the one or more T-cell antigens comprise two or more CD8+ T cell antigens each independently encoded by: UL32, UL55, UL75 UL122, UL123, or combinations thereof.

26. The polyribonucleotide of claim 21, wherein the one or more T cell antigens comprises two or more CD4+ T cell antigens each independently encoded by: UL44, UL55, UL75, U83, UL122, UL123, or combinations thereof.

27. The polyribonucleotide of claim 21, wherein the one or more T cell antigens comprises two or more CD4+ T cell antigens each independently encoded by: UL32, UL44, UL55, UL75, UL83, UL122, UL123, or combinations thereof.

28. The polyribonucleotide of claim 21, wherein the latent virus is norovirus.

29. The polyribonucleotide of claim 28, wherein the norovirus is of norovirus clade GI, GII.P2, GII.P4, GII.P7, GII.P12, GII.P16, GII.P17, GIX, or combinations thereof.

30. The polyribonucleotide of claim 28 or 29, wherein the one or more T-cell antigens comprise two or more T-cell antigens each independently encoded by a gene listed in Tables 12A-12D and/or Tables 13A-13B herein.

31. The polyribonucleotide of claim 28 or 29, wherein the one or more T-cell antigens comprise two or more T-cell antigens each independently having an amino acid sequence selected from Table 14A-14N, Table 15A, and/or Tables 16A-16P herein.

32. The polyribonucleotide of claim 28 or 29, wherein the one or more T-cell antigens comprise two or more CD8+ T cell antigens each independently encoded by: NTPase, Nterm, Pro, RdRp or combinations thereof.

33. The polyribonucleotide of claim 28 or 29, wherein the one or more T cell antigens comprises two or more CD4+ T cell antigens each independently encoded by: Nterm, VP1, Pro, or combinations thereof.

34. The polyribonucleotide of any one of claims 1-33, wherein the polypeptide comprises an MHC class I trafficking signal domain (MITD).

35. The polyribonucleotide of claim 34, wherein the MITD comprises or consists of the amino acid sequence of IVGIVAGLAVLAVVVIGAVVATVMCRRKSSGGKGGSYSQAASSDSAQGSDVSLTA.

36. The polyribonucleotide of claim 34 or 35, wherein the MITD is located at the C-terminus of the polypeptide.

37. The polyribonucleotide of any one of claims 1-36, wherein the polypeptide comprises a secretory signal.

38. The polyribonucleotide of claim 37, wherein the secretory signal comprises or consists of a heterologous secretory signal.

39. The polyribonucleotide of claim 38, wherein the heterologous secretory signal comprises or consists of a non-human secretory signal.

40. The polyribonucleotide of claim 38, wherein the heterologous secretory signal comprises or consists of a viral secretory signal.

41. The polyribonucleotide of claim 40, wherein the viral secretory signal comprises or consists of an HSV secretory signal.

42. The polyribonucleotide of claim 41, wherein the HSV secretory signal comprises or consists of an HSV-1 or HSV-2 secretory signal.

43. The polyribonucleotide of claim 41 or 42, wherein the HSV secretory signal comprises or consists of an HSV glycoprotein D (gD) secretory signal.

44. The polyribonucleotide of claim 43, wherein the HSV glycoprotein D (gD) secretory signal comprises or consists of an HSV-1 gD secretory signal.

45. The polyribonucleotide of claim 44, wherein the HSV-1 gD secretory signal comprises or consists of the amino acid sequence of MGGAAARLGAVILFVVIVGLHGVRSKY.

46. The polyribonucleotide of claim 44, wherein the HSV-1 gD secretory signal comprises or consists of the amino acid sequence of MGGAAARLGAVILFVVIVGLHGVRGKY.

47. The polyribonucleotide of claim 40, wherein the secretory signal comprises or consists of an Ebola virus secretory signal.

48. The polyribonucleotide of claim 47, wherein the Ebola virus secretory signal comprises or consists of an Ebola virus spike glycoprotein (SGP) secretory signal.

49. The polyribonucleotide of claim 48, wherein the Ebola virus SGP secretory signal comprises or consists of the amino acid sequence of MGVTGILQLPRDRFKRTSFFLWVIILFQRTFS.

50. The polyribonucleotide of any one of claims 37-49, wherein the secretory signal is located at the N-terminus of the polypeptide.

51. The polyribonucleotide of any one of claims 1-50, wherein the polypeptide comprises a transmembrane domain.

52. The polyribonucleotide of claim 51, wherein the transmembrane domain comprises or consists of a heterologous transmembrane domain.

53. The polyribonucleotide of claim 51 or 52, wherein the transmembrane domain comprises or consists of a transmembrane domain of Hemagglutinin (HA) of Influenza virus, Env of HIV-1, equine infectious anaemia virus (EIAV), murine leukaemia virus (MLV), mouse mammary tumor virus, G protein of vesicular stomatitis virus (VSV), Rabies virus, HSV virus, or a seven transmembrane domain receptor.

54. The polyribonucleotide of claim 52 or 53, wherein the heterologous transmembrane domain does not comprise a hemagglutinin transmembrane domain.

55. The polyribonucleotide of claim 52 or 53, wherein the heterologous transmembrane domain comprises or consists of a non-human transmembrane domain.

56. The polyribonucleotide of claim 52 or 53, wherein the heterologous transmembrane domain comprises or consists of a viral transmembrane domain.

57. The polyribonucleotide of claim 56, wherein the heterologous transmembrane domain comprises or consists of an HSV transmembrane domain.

58. The polyribonucleotide of claim 57, wherein the HSV transmembrane domain comprises or consists of an HSV-1 or HSV-2 transmembrane domain.

59. The polyribonucleotide of claim 57 or 58, wherein the HSV transmembrane domain comprises or consists of an HSV gD transmembrane domain.

60. The polyribonucleotide of claim 59, wherein the HSV gD transmembrane domain comprises or consists of an HSV-1 gD transmembrane domain.

61. The polyribonucleotide of claim 60, wherein the HSV-1 gD transmembrane domain comprises or consists of the amino acid sequence of GLIAGAVGGSLLAALVICGIVYWMRRHTQKAPKRIRLPHIR.

62. The polyribonucleotide of claim 51 or 52, wherein the transmembrane domain comprises or consists of a human transmembrane domain.

63. The polyribonucleotide of claim 62, wherein the human transmembrane domain comprises or consists of a human decay accelerating factor glycosylphosphatidylinositol (hDAF-GPI) anchor region.

64. The polyribonucleotide of claim 63, wherein the hDAF-GPI anchor region comprises or consists of the amino acid sequence of PNKGSGTTSGTTRLLSGHTCFTLTGLLGTLVTMGLLT.

65. The polyribonucleotide of any one of claims 1-36, wherein the polypeptide comprises an HSV-1 gD secretory signal and an HSV-1 gD transmembrane domain.

66. The polyribonucleotide of any one of claims 1-36 and 51-65, wherein the polypeptide does not comprise a secretory signal.

67. The polyribonucleotide of any one of claims 1-50 and 66, wherein the polypeptide does not comprise a transmembrane domain.

68. The polyribonucleotide of any one of claims 1-67, wherein the polypeptide comprises one or more linkers.

69. The polyribonucleotide of claim 68, wherein the one or more linkers comprise or consist of the amino acid sequence of GGSGGGGSGG.

70. The polyribonucleotide of claim 68, wherein the one or more linkers comprise or consist of the amino acid sequence of GGGS.

71. The polyribonucleotide of claim 68, wherein the one or more linkers comprise or consist of the amino acid sequence of GGGGSGGGGSGGGGS.

72. The polyribonucleotide of claim 68, wherein the one or more linkers comprise or consist of the amino acid sequence of AGNRVRRSVG.

73. The polyribonucleotide of any one of claims 68-72, wherein the one or more linkers is positioned between two antigens or two T-cell antigens.

74. The polyribonucleotide of any one of claims 1-73, wherein the polyribonucleotide is an isolated polyribonucleotide.

75. The polyribonucleotide of any one of claims 1-74, wherein the polyribonucleotide is an engineered polyribonucleotide.

76. The polyribonucleotide of any one of claims 1-75, wherein the polyribonucleotide is a codon-optimized polyribonucleotide.

77. An RNA construct comprising in 5′ to 3′ order:
a. a 5′ UTR that comprises or consists of a modified human alpha-globin 5′-UTR;
b. a polyribonucleotide of any one of claims 1-76 and 164-169;
c. a 3′ UTR that comprises or consists of a first sequence from the amino terminal enhancer of split (AES) messenger RNA and a second sequence from the mitochondrial encoded 12S ribosomal RNA; and
d. a polyA tail sequence.

78. The RNA construct of claim 77, wherein the 5′ UTR comprises or consists of a ribonucleic acid sequence of AGAAUAAACUAGUAUUCUUCUGGUCCCCACAGACUCAGAGAGAACCCGCCAC C.

79. The RNA construct of claim 77 or 78, wherein the 3′ UTR comprises or consists of a ribonucleic acid sequence according to CUGGUACUGCAUGCACGCAAUGCUAGCUGCCCCUUUCCCGUCCUGGGUACCCC GAGUCUCCCCCGACCUCGGGUCCCAGGUAUGCUCCCACCUCCACCUGCCCCAC UCACCACCUCUGCUAGUUCCAGACACCUCCCAAGCACGCAGCAAUGCAGCUCA AAACGCUUAGCCUAGCCACACCCCCACGGGAAACAGCAGUGAUUAACCUUUA GCAAUAAACGAAAGUUUAACUAAGCUAUACUAACCCCAGGGUUGGUCAAUUU CGUGCCAGCCACACC.

80. The RNA construct of any one of claims 77-79, wherein the polyA tail sequence is a split polyA tail sequence.

81. The RNA construct of claim 80, wherein the split polyA tail sequence comprises or consists of a ribonucleic acid sequence of AAAAAAAAAAAAAAAAAAAAAAAAAAAAAAGCAUAUGACUAAAAAAAAAAA AAAAAAAAAAAAAAAAAAAAAAAAAAAAAAAAAAAAAAAAAAAAAAAAAAA AAAAAAAA.

82. The RNA construct of any one of claims 77-81, further comprising a 5′ cap.

83. The RNA construct of any one of claims 77-82, further comprises a cap proximal sequence comprising positions +1, +2, +3, +4, and +5 of the polyribonucleotide.

84. The RNA construct of claim 82 or 83, wherein the 5′ cap comprises or consists of m7(3′OMeG)(5′)ppp(5′)(2′OMeA₁)pG₂, wherein A₁ is position +1 of the polyribonucleotide, and G₂ is position +2 of the polyribonucleotide.

85. The RNA construct of claim 83 or 84, wherein the cap proximal sequence comprises A₁ and G₂ of the Cap1 structure, and a sequence comprising: A₃A₄U₅ at positions +3, +4 and +5 respectively of the polyribonucleotide.

86. A composition comprising one or more polyribonucleotides of any one of claims 1-76 and 164-169.

87. A composition comprising one or more RNA constructs of any one of claims 77-85.

88. The composition of claim 86 or 87, wherein the composition further comprises lipid nanoparticles, polyplexes (PLX), lipidated polyplexes (LPLX), liposomes, or polysaccharide nanoparticles, wherein the one or more polyribonucleotides are fully or partially encapsulated within the lipid nanoparticles, polyplexes (PLX), lipidated polyplexes (LPLX), liposomes, or polysaccharide nanoparticles.

89. The composition of claim 86 or 87, wherein the composition further comprises lipid nanoparticles, wherein the one or more polyribonucleotides are encapsulated within the lipid nanoparticles.

90. The composition of claim 88 or 89, wherein the lipid nanoparticles target liver cells.

91. The composition of claim 88 or 89, wherein the lipid nanoparticles target secondary lymphoid organ cells.

92. The composition of any one of claims 88-91, wherein the lipid nanoparticles are cationic lipid nanoparticles.

93. The composition of any one of claims 88-92, wherein the lipid nanoparticles each comprise: a polymer-conjugated lipid; a cationically ionizable lipid; and one or more neutral lipids.

94. The composition of claim 93, wherein the polymer-conjugated lipid comprises a PEG-conjugated lipid.

95. The composition of claim 93 or 94, wherein the polymer-conjugated lipid comprises 2-[(polyethylene glycol)-2000]—N,N-ditetradecylacetamide.

96. The composition of any one of claims 93-95, wherein the one or more neutral lipids comprise 1,2-Distearoyl-sn-glycero-3-phosphocholine (DPSC).

97. The composition of any one of claims 93-96, wherein the one or more neutral lipids comprise cholesterol.

98. The composition of any one of claims 93-97, wherein the cationically ionizable lipid comprises [(4-Hydroxybutyl)azanediyl]di(hexane-6,1-diyl) bis(2-hexyldecanoate).

99. The composition of any one of claims 88-98, wherein the lipid nanoparticles have an average diameter of about 50-150 nm.

100. A pharmaceutical composition comprising the composition of any one of claims 86-99 and at least one pharmaceutically acceptable excipient.

101. The pharmaceutical composition of claim 100, wherein the pharmaceutical composition comprises a cryoprotectant, optionally wherein the cryoprotectant is or comprises sucrose.

102. The pharmaceutical composition of claim 100 or 101, wherein the pharmaceutical comprises an aqueous buffered solution, optionally wherein the aqueous buffered solution comprises one or more of Tris base, Tris HCl, NaCl, KCl, Na₂HPO₄, and KH₂PO₄.

103. A combination comprising:
a. a first pharmaceutical composition comprising a first polyribonucleotide, wherein the first polyribonucleotide is a polyribonucleotide according to any one of claims 1-76 and 164-169; and
b. a second pharmaceutical composition comprising a second polyribonucleotide, wherein the second polyribonucleotide encodes a second polypeptide, and the second polypeptide comprises one or more antigens from the latent virus, and the one or more antigens of the second polypeptide are different than the one or more antigens of the first polypeptide.

104. The combination of claim 103, wherein the one or more antigens of the second polypeptide comprise one or more antigens that are expressed during an initial infection with the latent virus.

105. The combination of claim 103 or 104, wherein the one or more antigens of the second polypeptide comprise one or more antigens that are expressed during productive replication of the latent virus.

106. The combination of any one of claims 103-105, wherein the one or more antigens of the second polypeptide are or comprise one or more T-cell antigens.

107. The combination of any one of claims 103-106, wherein the one or more antigens of the second polypeptide are or comprise one or more B-cell antigens.

108. The combination of any one of claims 103-107, wherein the one or more antigens of the second polypeptide are or comprise antigenic polypeptide regions of the latent virus or portions thereof.

109. The combination of any one of claims 103-108, wherein the latent virus is capable of infecting a human.

110. The combination of any one of claims 103-109, wherein the one or more antigens of the second polypeptide are or comprise one or more antigens from one or more surface proteins of Varicella-Zoster virus.

111. The combination of claim 110, wherein the one or more surface proteins of Varicella-Zoster virus are or comprise glycoprotein E, glycoprotein B, glycoprotein H, glycoprotein L, glycoprotein C, glycoprotein M, glycoprotein N, or combinations thereof.

112. The combination of claim 110 or 111, wherein the first polyribonucleotide is a polyribonucleotide according to any one of claims 15-20.

113. The combination of any one of claims 103-109, wherein the one or more antigens of the second polypeptide are or comprise one or more antigens from one or more surface proteins of CMV.

114. The combination of claim 113, wherein the one or more surface proteins of CMV are or comprise glycoprotein B, glycoprotein M, glycoprotein N, glycoprotein H, glycoprotein L, glycoprotein O, UL128, UL129, UL130, UL131, or combinations thereof.

115. The combination of claim 113 or 114, wherein the first polyribonucleotide is a polyribonucleotide according to any one of claims 22-27.

116. The combination of any one of claims 103-109, wherein the one or more antigens of the second polypeptide are or comprise one or more antigens from one or more surface proteins of norovirus.

117. The combination of claim 116, wherein the one or more surface proteins of norovirus are or comprise VP1.

118. The combination of claim 116 or 117, wherein the first polyribonucleotide is a polyribonucleotide according to any one of claims 29-33.

119. The combination of any one of claims 103-118, wherein the first pharmaceutical composition and the second pharmaceutical composition are not in the same composition.

120. A method comprising administering a polyribonucleotide of any one of claims 1-76 and 164-165 to a subject.

121. A method comprising administering an RNA construct of any one of claims 77-85 to a subject.

122. A method comprising administering a composition of any one of claims 86-99 to a subject.

123. A method comprising administering one or more doses of the pharmaceutical composition of any one of claims 100-102 to a subject.

124. The pharmaceutical composition of any one of claims 100-102 for use in the treatment of a latent viral infection comprising administering one or more doses of the pharmaceutical composition to a subject.

125. The pharmaceutical composition of any one of claims 100-102 for use in the prevention of a latent viral infection comprising administering one or more doses of the pharmaceutical composition to a subject.

126. The method of claim 123 or the pharmaceutical composition for use of claims 124-125, comprising administering two or more doses of the pharmaceutical composition to a subject.

127. The method of claim 123 or 126, or the pharmaceutical composition for use of any one of claims 124-126, comprising administering three or more doses of the pharmaceutical composition to a subject.

128. The method of claim 123, 126, or 127, or the pharmaceutical composition for use of any one of claims 124-127, wherein the subject is suffering from a viral infection.

129. The method of claim 128, or the pharmaceutical composition for use of claim 128, wherein the viral infection is an initial infection.

130. The method of any one of claims 123 and 126-127, or the pharmaceutical composition for use of any one of claims 124-127, wherein the subject intends to be present within a geographical region that has a high viral prevalence within the next three months.

131. The method of claim 130, or the pharmaceutical composition for use of claim 130, wherein the high viral prevalence is greater than 10% of the population.

132. The method of any one of claims 123 and 126-131, or the pharmaceutical composition for use of any one of claims 124-131, wherein the subject has previously been treated for a viral infection with a different pharmaceutical composition.

133. The method of any one of claims 123 and 126-132, or the pharmaceutical composition for use of any one of claims 124-132, wherein the subject is a human subject.

134. A method comprising administering a combination of any one of claims 103-119 to a subject.

135. The method of claim 134, wherein the first pharmaceutical composition and the second pharmaceutical composition are administered on different days.

136. The method of claim 134, wherein the first pharmaceutical composition and the second pharmaceutical composition are administered on the same day.

137. The method of any one of claims 134-136, wherein the first pharmaceutical composition and the second pharmaceutical composition are administered at different locations on the subject's body.

138. The method of any one of claims 134-137, wherein the first pharmaceutical composition and the second pharmaceutical composition are each administered at a location on the subject's body that is in immunological communication with a distinct lymph node.

139. The method of claim 138, wherein the first pharmaceutical composition and the second pharmaceutical composition are administered at different arms.

140. The method of claim 139, wherein the first pharmaceutical composition and the second pharmaceutical composition are administered at different legs.

141. The method of any one of claims 134-140, wherein the method is a method of treating a latent viral infection.

142. The method of any one of claims 134-140, wherein the method is a method of preventing a latent viral infection.

143. The method of any one of claims 134-142, wherein the subject is suffering from a viral infection.

144. The method of claim 143, wherein the viral infection is an initial infection.

145. The method of any one of claims 134-142, wherein the subject intends to be present within a geographical region that has a high viral prevalence within the next three months.

146. The method of claim 145, wherein the high viral prevalence is greater than 10% of the population.

147. The method of any one of claims 134-146, wherein the subject has previously been treated for a viral infection with a different pharmaceutical composition.

148. The method of any one of claims 134-147, wherein the subject is a human.

149. The method of any one of claims 120-148, wherein administration induces an anti-viral immune response in the subject.

150. The method of claim 149, wherein the anti-viral immune response in the subject comprises an adaptive immune response.

151. The method of claim 149 or 150, wherein the anti-viral immune response in the subject comprises a T-cell response.

152. The method of claim 151, wherein the T-cell response is or comprises a CD4+ T cell response.

153. The method of claim 151 or 152, wherein the T-cell response is or comprises a CD8+ T cell response.

154. The method of any one of claims 149-153, wherein the anti-viral immune response comprises a B-cell response.

155. The method of any one of claims 149-154, wherein the anti-viral immune response comprises production of antibodies directed against the one or more antigens.

156. Use of the pharmaceutical composition of any one of claims 100-102 in the treatment of a virus infection.

157. Use of the pharmaceutical composition of any one of claims 100-102 in the prevention of a virus infection.

158. Use of the pharmaceutical composition of any one of claims 100-102 in inducing an anti-viral immune response in a subject.

159. A polypeptide encoded by a polyribonucleotide of any one of claims 1-76 and 164-169.

160. A polypeptide encoded by an RNA construct of any one of claims 77-85.

161. A host cell comprising a polyribonucleotide of any one of claims 1-76 and 164-169.

162. A host cell comprising an RNA construct of any one of claims 77-85.

163. A host cell comprising a polypeptide of claim 159 or 160.

164. A polyribonucleotide encoding a polypeptide, wherein the polypeptide comprises an HSV-1 gD secretory signal, one or more viral antigens, and an HSV-1 gD transmembrane domain.

165. The polyribonucleotide of claim 164, wherein the HSV-1 gD secretory signal comprises or consists of the amino acid sequence of MGGAAARLGAVILFVVIVGLHGVRSKY.

166. The polyribonucleotide of claim 164, wherein the HSV-1 gD secretory signal comprises or consists of the amino acid sequence of MGGAAARLGAVILFVVIVGLHGVRGKY.

167. The polyribonucleotide of any one of claims 164-166, wherein the HSV-1 gD transmembrane domain comprises or consists of the amino acid sequence of GLIAGAVGGSLLAALVICGIVYWMRRHTQKAPKRIRLPHIR.

168. The polyribonucleotide of any one of claims 164-167, wherein the one or more viral antigens are from a latent virus of the Herpesviridae, Papillomaviridae, Parvoviridae, or Adenoviridae family.

169. The polyribonucleotide of claim 169, wherein the latent virus is HSV-1, HSV-2, VZV, Human Immunodeficiency Virus (HIV), Epstein-Barr Virus (EBV), CMV, HHV-6, HHV-7, KSHV (HHV-8), JC virus (JCV), BK virus (BKV), parvovirus, or adenovirus.
